(19) United States
(12) Patent Application Publication
(10) Pub. No.: US 2026/0184712 A1
(43) Pub. Date: Jul. 2, 2026

(54) Heterocyclic Compounds as ENT Inhibitors and Compounds for Use in the Treatment of Cancers

(71) Applicant: iTeos Belgium SA, Gosselies (BE)

(72) Inventors: Michael Deligny, Rhode Saint Genese (BE); Steven Van der Plas, Steenhuffel (BE); Erica Joke Katelijne Heleen Houthuys, Enghien (BE); Michael Rowley, Chelmsford (GB)

(73) Assignee: iTeos Belgium SA, Gosselies (BE)

(21) Appl. No.: 19/241,489

(22) Filed: Jun. 18, 2025

Related U.S. Application Data
(63) Continuation of application No. PCT/IB2023/063020, filed on Dec. 20, 2023.
(60) Provisional application No. 63/476,194, filed on Dec. 20, 2022.
(60) Provisional application No. 63/609,006, filed on Dec. 12, 2023.

(51) Int. Cl. C07D 473/30; C07D 473/00; C07D 473/04; C07D 473/18; C07D 473/34; C07D 473/38; C07D 487/04; C07D 519/00; C07H 19/167; C07H 19/173

(52) U.S. Cl. CPC C07D 473/30; C07D 473/00; C07D 473/04; C07D 473/18; C07D 473/34; C07D 473/38; C07D 487/04; C07D 519/00; C07H 19/167; C07H 19/173

(57) ABSTRACT

The present disclosure relates to certain heterocyclic compounds, including compounds of Formula (I):

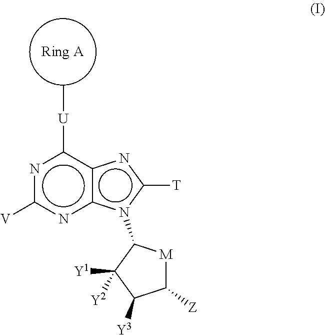

or pharmaceutically acceptable salts, hydrates, or solvates thereof. The disclosure further relates to the use of the compounds disclosed as inhibitors of equilibrative nucleoside transporters (ENTs). The disclosure also relates to the use of the compounds disclosed for the treatment and/or prevention of cancer.

### CROSS-REFERENCE TO RELATED APPLICATIONS

This application is a continuation of PCT Application No. PCT/IB2023/063020, filed Dec. 20, 2023, which claims the benefit of priority to U.S. Provisional Application No. 63/476,194, filed Dec. 20, 2022, and to U.S. Provisional Application No. 63/609,006, filed Dec. 12, 2023, both of which are incorporated by reference herein in their entirety.

### FIELD

Disclosed herein are certain heterocyclic compounds, including compounds of Formula (I):

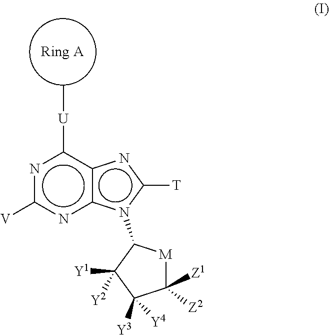

or pharmaceutically acceptable salts, hydrates, or solvates thereof. Compounds of the present disclosure are inhibitors of equilibrative nucleoside transporters, such as ENT1, and are useful as therapeutic compounds, for example in the treatment and/or prevention of cancers.

### BACKGROUND AND SUMMARY

The equilibrative nucleoside transporter (ENT) family, also known as SLC29, is a group of plasmalemmal transport proteins which transport nucleoside substrates into cells. There are four known ENTs, designated ENT1, ENT2, ENT3, and ENT4.

One of the endogenous substrates for ENTs is adenosine, a potent physiological and pharmacological regulator of numerous functions. Cellular signaling by adenosine occurs through four known G-protein-coupled adenosine receptors A1, A2A, A2B, and A3. By influencing the concentration of adenosine available to these receptors, ENTs fulfill important regulatory roles in different physiological processes, such as modulation of coronary blood flow, inflammation, and neurotransmission (Griffith D A and Jarvis S M, Biochim Biophys Acta, 1996, 1286, 153-181; Shryock J C and Belardinelli L, Am J Cardiol, 1997, 79(12A), 2-10; Anderson C M et al., J Neurochem, 1999, 73, 867-873).

Adenosine is also a potent immunosuppressive metabolite that is often found elevated in the extracellular tumor microenvironment (TME) (Blay J et al., Cancer Res, 1997, 57, 2602-2605). Extracellular adenosine is generated mainly by the conversion of ATP by the ectonucleotidases CD39 and CD73 (Stagg J and Smyth M J, Oncogene, 2010, 2, 5346-5358). Adenosine activates four G-protein-coupled receptor subtypes (A1, A2A, A2B, and A3). In particular, activation of the A2A receptor is believed to be the main driver of innate and adaptive immune cell suppression leading to suppression of antitumor immune responses (Ohta and Sitkovsky, Nature, 2001, 414, 916-920; Stagg and Smyth, Oncogene, 2010, 2, 5346-5358; Antonioli L et al., Nature Reviews Cancer, 2013, 13, 842-857; Cekic C and Linden J, Nature Reviews, Immunology, 2016, 16, 177-192; Allard B et al., Curr Op Pharmacol, 2016, 29, 7-16; Vijayan D et al., Nature Reviews Cancer, 2017, 17, 709-724).

The Applicant previously evidenced in PCT/EP2019/076244 that adenosine as well as ATP profoundly suppress T cell proliferation and cytokine secretion (IL-2), and strongly reduce T cell viability. Adenosine- and ATP-mediated suppression of T cell viability and proliferation were successfully restored by using ENTs inhibitors. Moreover, the use of an ENT inhibitor in combination with an adenosine receptor antagonist enabled to restore not only adenosine- and ATP-mediated suppression of T cell viability and proliferation, but also restored T cell cytokine secretion. These results showed that ENTs inhibitors either alone or in combination with an adenosine receptor antagonist may be useful for the treatment of cancers.

A variety of drugs such as dilazep, dipyridamole, and draflazine interact with ENTs and alter adenosine levels, and were developed for their cardioprotective or vasodilatory effects.

Currently, two non-selective ENT1 inhibitors (dilazep and dipyridamole) are on the market (Vlachodimou et ah, Bio-Chemical Pharmacology, 2020, 172, 113747). However, their binding kinetics are unknown; moreover, there is still a need for more potent ENTs inhibitors, and especially ENT1 inhibitors to be used for the treatment of cancers, either alone or in combination with an adenosine receptor antagonist.

In one aspect, the present disclosure relates to compounds of Formula (I):

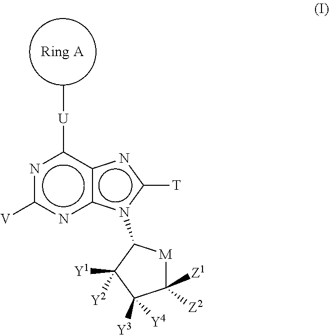

or pharmaceutically acceptable salts, hydrates, or solvates thereof, wherein U is a direct bond or is chosen from: (i) —O—, (ii) -alkoxy-, (iii) -(alkyl)O(alkyl)-, (iv) -alkyl-, (v) -alkenyl-, (vi) -alkyl-S— wherein alkyl is attached to Ring A, (vii) —SO₂NR¹— wherein the N is attached to Ring A, (viii) -alkyl-SO₂—NR¹— wherein the N is attached to Ring A, (ix) -alkyl-NR¹— wherein alkyl is attached to Ring A, (x) —NR¹—, (xi) —C(O)NR¹— wherein the N is attached to Ring A, (xii) —C(O)NR¹-alkyl- wherein the alkyl is attached to Ring A, and (xiii) —CO—; and wherein Ring A, T, V, M, Y¹, Y², Y³, Y⁴, Z¹, and Z² are hereafter defined.

In one aspect, the present disclosure relates to compounds of Formula (Ia):

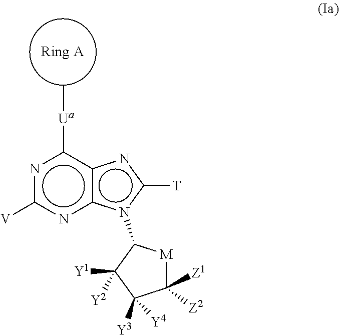

or a pharmaceutically acceptable salt, hydrate, or solvate thereof, wherein U^{a} is chosen from: (i) —O—, (ii) -alkoxy-, and (iii) -(alkyl)O(alkyl)-; and wherein Ring A, T, V, M, Y¹, Y², Y³, Y⁴, Z¹, and Z² are hereafter defined.

In one aspect, the present disclosure relates to compounds of Formula (Ib):

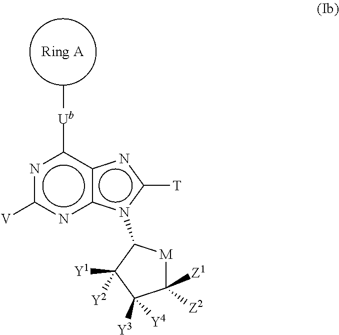

or a pharmaceutically acceptable salt, hydrate, or solvate thereof, wherein U^{b} is: (i) -alkyl- or (ii) -alkenyl-; and wherein Ring A, T, V, M, Y¹, Y², Y³, Y⁴, Z¹, and Z² are hereafter defined.

In one aspect, the present disclosure relates to compounds of Formula (Ic):

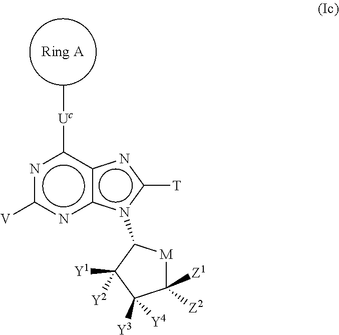

or a pharmaceutically acceptable salt, hydrate, or solvate thereof, wherein U^{c} is chosen from: (i) -alkyl-NR¹— wherein alkyl is attached to Ring A, (ii) —NR¹—, (iii) —CO—, (iv) —C(O)NR¹— wherein the N is attached to Ring A, and (v) —C(O)NR¹-alkyl- wherein the alkyl is attached to Ring A; and wherein Ring A, T, V, M, Y¹, Y², Y³, Y⁴, Z¹, and Z² are hereafter defined.

In one aspect, the present disclosure relates to compounds of Formula (Id):

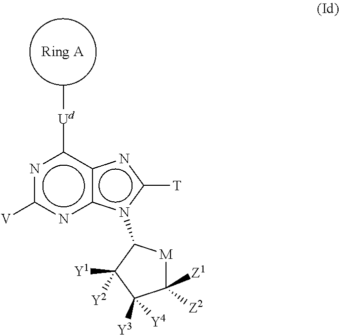

or a pharmaceutically acceptable salt, hydrate, or solvate thereof, wherein U^{d} is chosen from: (i) -alkyl-S— wherein alkyl is attached to Ring A, (ii) —SO₂NR¹— wherein the N is attached to Ring A, and (iii) -alkyl-SO₂—NR¹— wherein the N is attached to Ring A; and wherein Ring A, T, V, M, Y¹, Y², Y³, Y⁴, Z¹, and Z² are hereafter defined.

In one aspect, the present disclosure relates to compounds of Formula (Ie):

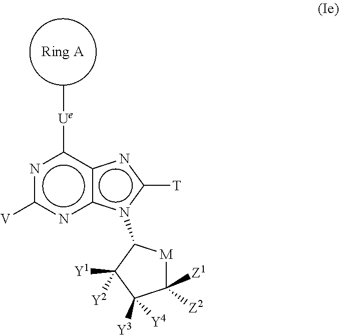

or a pharmaceutically acceptable salt, hydrate, or solvate thereof, wherein U^{e} is a direct bond; and wherein Ring A, T, V, M, Y¹, Y², Y³, Y⁴, Z¹, and Z² are hereafter defined.

In one aspect, the present disclosure relates to compounds disclosed in Table 1 and the Examples.

The present disclosure also relates to a pharmaceutical composition comprising at least one compound chosen from those disclosed herein, pharmaceutically acceptable salts, hydrates, and solvates thereof, and a pharmaceutically acceptable excipient.

The disclosure further relates to a method of inhibiting ENT1 in a patient need thereof, comprising administering to the patient an effective amount of at least one compound chosen from those disclosed herein.

The disclosure also relates to a method of treating cancer in a patient need thereof, comprising administering to the patient an effective amount of at least one compound chosen from those disclosed herein.

The disclosure is also directed to a method of treating cancer in a patient need thereof, comprising administering to the patient a combination of at least one compound chosen from those disclosed herein and an adenosine receptor antagonist.

The disclosure further relates to a kit of parts comprising: (i) a first part comprising an effective amount of at least one compound chosen from those disclosed herein; and (ii) a second part comprising an effective amount of an adenosine receptor antagonist.

### BRIEF DESCRIPTION OF THE DRAWINGS

FIG. 1A depicts a graph of log concentration of the ENT1 inhibitors NBMR and Compound 75 (M) versus percent proliferation. Purified human T cells were activated with anti-CD3/CD28 dynabeads in the presence of ATP (100 μM) as a source of adenosine for 96 hours and then proliferation was assessed by CFSE dilution, as discussed in Biological Example II.1.c.

FIG. 1B depicts a graph of log concentration of the ENT1 inhibitors NBMR and Compound 75 (M) verse percent proliferation. The experiment was performed the same as in the experiment in FIG. 1A with the addition of Human Serum Albumine (HAS) and α-1-Acid Glycoprotein (AAG) to the culture medium (final concentration 2 and 0.1%, respectively), as discussed in Biological Example II.1.c.

### DETAILED DESCRIPTION

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. Standard techniques may be used for chemical synthesis and chemical analysis. As appropriate, procedures involving the use of commercially available kits and reagents are generally carried out in accordance with manufacturer-defined protocols and conditions unless otherwise noted.

For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, T, John Wiley & Sons, New York: 2001.

Unless otherwise indicated, the following terms have the following meanings:

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “aldehyde” refers to a —CHO group.

The term “alkoxy” refers to a —O-alkyl group wherein alkyl is as herein defined. In some embodiments, the alkoxy group has monovalency. In some embodiments, the alkoxy group has bivalency. An alkoxy group can be unsubstituted or substituted with, for example, any of the groups below in the definition of alkyl.

An “alkyl” group is a saturated, partially saturated, or unsaturated straight chain or branched non-cyclic hydrocarbon having from 1 to 10 carbon atoms (C₁-C₁₀ alkyl), from 1 to 8 carbons (C₁-C₈ alkyl), from 1 to 6 (C₁-C₆ alkyl), 1 to 4 (C₁-C₄ alkyl), 1 to 3 (C₁-C₃ alkyl), or 2 to 6 (C₂-C₆ alkyl) carbon atoms. In some embodiments, the alkyl group has monovalency. Examples of alkyl groups with monovalency include, but are not limited to, —CH₃, —CH₂CH₃, —CH₂CH₂CH₃, —CH₂CH₂CH₃, —CH₂(CH₂)₂CH₃, —CH₂CH(CH₃)CH₃, —CH₂(CH₂)₃CH₃, —CH₂(CH₂)₄CH₃, —CH₂(CH₂)₅CH₃, —CH₂(CH₂)₆CH₃, and the like. In some embodiments, the alkyl group has bivalency. Examples of alkyl groups with bivalency include, but are not limited to, —CH₂—, —CH₂CH₂—, —CH₂CH₂CH₂—, —CH₂CH(CH₃)—, —CH₂(CH₂)₂CH₂—, —CH₂CH(CH₃)CH₂—, —CH₂(CH₂)₃CH₂—, —CH₂(CH₂)₄CH₂—, —CH₂(CH₂)₅CH₂—, —CH₂(CH₂)₆CH₂—, and the like. In some embodiments, the alkyl group is a saturated alkyl group. Representative saturated alkyl groups include -methyl, -ethyl, -n-propyl, -n-butyl, -n-pentyl, -n-hexyl, -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of monovalent unsaturated alkyl groups include, but are not limited to, vinyl, allyl, —CH═CH(CH₃), —CH═C(CH₃)₂, —C(CH₃)=CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. In some embodiments, alkyl is an optionally substituted C₁-C₆ alkyl.

An alkyl group can be substituted or unsubstituted. An alkyl group can be optionally substituted by, for example, 1, 2, or 3 independently chosen substituents, such as, -halo, —OH, optionally substituted alkoxy, —NO₂, —CN, optionally substituted —C(O)alkyl, optionally substituted —C(O)—O-alkyl, optionally substituted amino, optionally substituted amido (e.g., —C(O)NH₂, —C(O)N(alkyl)₂, —NH(CO)(alkyl), —N(alkyl)(CO)(alkyl)), optionally substituted carboxy (e.g., —CO₂H, —CO₂(alkyl)), optionally substituted carbamyl (e.g., —C(O)NH₂, —C(O)N(alkyl)₂), optionally substituted thiol (e.g., —SH, —S(alkyl)), optionally substituted sulfonyl (e.g., —SO₂H, —SO₂(alkyl)), optionally substituted sulfonamido (e.g. SO₂NH₂, —SO₂(alkyl)), optionally substituted cycloalkyl, optionally substituted aryl, optionally substituted heterocyclyl, optionally substituted heteroaryl, optionally substituted —O(cycloalkyl), optionally substituted —O(aryl), optionally substituted —O(heterocyclyl), optionally substituted —O(heteroaryl), a phosphonate group, and a protected alcohol. When the substituent is further substituted, it may be substituted with 1, 2, or 3 groups independently chosen from -halo, —NO₂, —CN, -alkyl, -alkoxy, -cycloalkyl, -aryl, -heterocyclyl, and -heteroaryl.

In some embodiments, an alkyl group is optionally substituted with 1, 2, or 3 groups independently chosen from -halo, —OH, —CN, —C(O)alkyl, —C(O)—O-alkyl, —SO₂(alkyl), optionally substituted alkoxy, —O(aryl), -amido, -phosphonate group, and a protected alcohol.

The term “amido” refers to a —C(O)NR₂ group or a —NR(CO)R group wherein R is H or an optionally substituted alkyl. In some embodiments, the R in the amido group is H or C₁₋₃ alkyl.

The terms “amino” and “amine” refers to a —NH₂ group. When an amino (or amine) is said to be substituted, it refers to a —NH₂ group wherein one or both of the —H atoms have been replaced with another group. For example, in certain embodiments, one or both of the —H atoms can be replaced by a group independently chosen from -alkyl, -cycloalkyl, -aryl, -heterocyclyl, and -heteroaryl, any of which may be optionally substituted.

An “aryl” group is an aromatic carbocyclic group of from 6 to 14 carbon atoms (C₆-C₁₄ aryl) having a single ring (e.g., phenyl) or multiple fused rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Exemplary aryls include phenyl, biphenyl, naphthyl and the like.

An aryl group can be substituted or unsubstituted. An aryl group can be optionally substituted by, for example, 1, 2, or 3 independently chosen substituents, such as, -halo, —OH, optionally substituted alkyl, optionally substituted alkoxy, —NO₂, —CN, optionally substituted —C(O)alkyl, optionally substituted —C(O)—O-alkyl, optionally substituted amino, optionally substituted amido (e.g., —C(O)NH₂, —C(O)N(alkyl)₂, —NH(CO)(alkyl), —N(alkyl)(CO)(alkyl)), optionally substituted carboxy (e.g., —CO₂H, —CO₂(alkyl)), optionally substituted carbamyl (e.g., —C(O)NH₂, —C(O)N(alkyl)₂), optionally substituted thiol (e.g., —SH, —S(alkyl)), optionally substituted sulfonyl (e.g., —SO₂H, —SO₂(alkyl)), optionally substituted sulfonamido (e.g. SO₂NH₂, —SO₂(alkyl)), optionally substituted cycloalkyl, optionally substituted aryl, optionally substituted heterocyclyl, optionally substituted heteroaryl, optionally substituted —O(cycloalkyl), optionally substituted —O(aryl), optionally substituted —O(heterocyclyl), optionally substituted —O(heteroaryl), a phosphonate group, and a protected alcohol. When the substituent is further substituted, it may be substituted with 1, 2, or 3 groups independently chosen from -halo, —NO₂, —CN, -alkyl, -alkoxy, -cycloalkyl, -aryl, -heterocyclyl, and -heteroaryl.

In some embodiments, an aryl group is substituted with 1, 2, or 3 groups independently chosen from -halo, —OH, —NO₂, —CN, optionally substituted alkyl, optionally substituted alkoxy, —C(O)alkyl, —C(O)—O-alkyl, —SO₂(alkyl), optionally substituted cycloalkyl, optionally substituted heteroaryl, and —O(aryl).

A “cycloalkyl” group is a saturated, or partially saturated cyclic alkyl group of from, for example, 3 to 10 carbon atoms (C₃-C₁₀ cycloalkyl) having a single cyclic ring or multiple fused or bridged rings. In some embodiments, the cycloalkyl group has 3 to 9 ring carbon atoms (C₃-C₉ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturared cycloalkyl groups include cyclohexenyl, cyclopentenyl, cyclohexadienyl, butadienyl, pentadienyl, hexadienyl, among others. In some embodiments, “cycloalkyl” encompasses any non-aromatic ring, even if fused to an aryl, regardless of the attachment to the remainder of the molecule. For example, “cycloalkyl” groups include groups such as

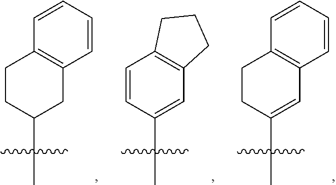

and the like.

A cycloalkyl group can be substituted or unsubstituted. A cycloalkyl group can be optionally substituted by, for example, 1, 2, or 3 independently chosen substituents, such as, -halo, —OH, optionally substituted alkyl, optionally substituted alkoxy, -oxo (—CO—), —NO₂, —CN, optionally substituted —C(O)alkyl, optionally substituted —C(O)—O-alkyl, optionally substituted amino, optionally substituted amido (e.g., —C(O)NH₂, —C(O)N(alkyl)₂, —NH(CO)(alkyl), —N(alkyl)(CO)(alkyl)), optionally substituted carboxy (e.g., —CO₂H, —CO₂(alkyl)), optionally substituted carbamyl (e.g., —C(O)NH₂, —C(O)N(alkyl)₂), optionally substituted thiol (e.g., —SH, —S(alkyl)), optionally substituted sulfonyl (e.g., —SO₂H, —SO₂(alkyl)), optionally substituted sulfonamido (e.g. SO₂NH₂, —SO₂(alkyl)), optionally substituted cycloalkyl, optionally substituted aryl, optionally substituted heterocyclyl, optionally substituted heteroaryl, optionally substituted —O(cycloalkyl), optionally substituted —O(aryl), optionally substituted —O(heterocyclyl), optionally substituted —O(heteroaryl), a phosphonate group, and a protected alcohol. When the substituent is further substituted, it may be substituted with 1, 2, or 3 groups independently chosen from -halo, —NO₂, —CN, -alkyl, -alkoxy, -cycloalkyl, -aryl, -heterocyclyl, and -heteroaryl.

In some embodiments, a cycloalkyl group is optionally substituted with 1, 2, or 3 groups independently chosen from -halo, —OH, —CN, —NO₂, optionally substituted alkyl, optionally substituted alkoxy, —C(O)alkyl, —SO₂(alkyl), optionally substituted aryl, —O(aryl), -amido, -phosphonate group, and a protected alcohol.

The term “halo” or “halogen” refers to fluoro, chloro, bromo, or iodo.

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 12 ring atoms, and in others from 5 to 12 or even 5 to 9 atoms in the ring portions of the groups. In some embodiments, heteroaryl groups have one to three heteroatoms, whereas other such groups have one to two heteroatoms or one heteroatom. Suitable heteroatoms include oxygen, sulfur and nitrogen. In some embodiments, the heteroaryl ring system is monocyclic or bicyclic. Non-limiting examples include but are not limited to, groups such as pyrrolyl, pyrazolyl, pyridinyl, pyrazinyl, pyrimidinyl, pyridazinyl, quinazolinyl, quinoxalinyl, quinolinyl, isoquinolinyl, thiazolyl, thiadiazolyl, triazolyl, tetrazolyl, triazinyl, thiopheny,

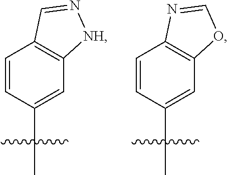 [SMILES: *c1ccc2cn[nH]c2c1 ; *c1ccc2ncoc2c1]

and the like.

A heteroaryl group can be substituted or unsubstituted. A heteroaryl group can be optionally substituted by, for example, 1, 2, or 3 independently chosen substituents, such as, -halo, —OH, optionally substituted alkyl, optionally substituted alkoxy, —NO₂, —CN, optionally substituted —C(O)alkyl, optionally substituted —C(O)—O-alkyl, optionally substituted amino, optionally substituted amido (e.g., —C(O)NH₂, —C(O)N(alkyl)₂, —NH(CO)(alkyl), —N(alkyl)(CO)(alkyl)), optionally substituted carboxy (e.g., —CO₂H, —CO₂(alkyl)), optionally substituted carbamyl (e.g., —C(O)NH₂, —C(O)N(alkyl)₂), optionally substituted thiol (e.g., —SH, —S(alkyl)), optionally substituted sulfonyl (e.g., —SO₂H, —SO₂(alkyl)), optionally substituted sulfonamido (e.g. SO₂NH₂, —SO₂(alkyl)), optionally substituted cycloalkyl, optionally substituted aryl, optionally substituted heterocyclyl, optionally substituted heteroaryl, optionally substituted —O(cycloalkyl), optionally substituted —O(aryl), optionally substituted —O(heterocyclyl), optionally substituted —O(heteroaryl), a phosphonate group, and a protected alcohol. When the substituent is further substituted, it may be substituted with 1, 2, or 3 groups independently chosen from -halo, —NO₂, —CN, -alkyl, -alkoxy, -cycloalkyl, -aryl, -heterocyclyl, and -heteroaryl.

In some embodiments, a heteroaryl group is optionally substituted with 1, 2, or 3 groups independently chosen from -halo, —OH, —CN, —NO₂, optionally substituted alkyl, optionally substituted alkoxy, —C(O)alkyl, —SO₂(alkyl), optionally substituted cycloalkyl, optionally substituted aryl, —O(aryl), -amido, -phosphonate group, and a protected alcohol.

A “heterocyclyl” is a non-aromatic cycloalkyl in which one to four of the ring carbon atoms are independently replaced with a heteroatom. In some embodiments, heterocyclyl groups include 3 to 11 ring members, whereas other such groups have 3 to 5, 3 to 6, 3 to 9, or 3 to 10 ring members. In some embodiments, heterocyclyl groups include one to three heteroatoms, whereas other such groups have one to two heteroatoms or one heteroatom. Heterocyclyls can be bonded to other groups at any ring atom (i.e., at any carbon atom or heteroatom of the heterocyclic ring). Heterocyclyl groups encompass saturated and partially saturated ring systems. Further, the term “heterocyclyl” encompasses any non-aromatic ring containing at least one heteroatom, which ring may be fused to an aryl or heteroaryl ring, regardless of the attachment to the remainder of the molecule. For example, “heterocyclyl” groups include groups such as

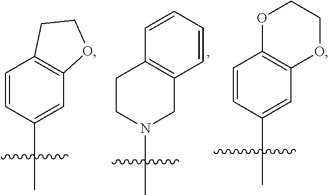

and the like. Heterocyclyl also includes bridged polycyclic ring systems containing a heteroatom. Representative examples of a heterocyclyl group include, but are not limited to, dioxolanyl, dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl and 1,1-dioxo-thiomorpholinyl.

A heterocyclyl group can be substituted or unsubstituted. A heterocyclyl group can be optionally substituted by, for example, 1, 2, or 3 independently chosen substituents, such as, -halo, —OH, optionally substituted alkyl, optionally substituted alkoxy, -oxo (—CO—), —NO₂, —CN, optionally substituted —C(O)alkyl, optionally substituted —C(O)—O-alkyl, optionally substituted amino, optionally substituted amido (e.g., —C(O)NH₂, —C(O)N(alkyl)₂, —NH(CO)(alkyl), —N(alkyl)(CO)(alkyl)), optionally substituted carboxy (e.g., —CO₂H, —CO₂(alkyl)), optionally substituted carbamyl (e.g., —C(O)NH₂, —C(O)N(alkyl)₂), optionally substituted thiol (e.g., —SH, —S(alkyl)), optionally substituted sulfonyl (e.g., —SO₂H, —SO₂(alkyl)), optionally substituted sulfonamido (e.g. SO₂NH₂, —SO₂(alkyl)), optionally substituted cycloalkyl, optionally substituted aryl, optionally substituted heterocyclyl, optionally substituted heteroaryl, optionally substituted —O(cycloalkyl), optionally substituted —O(aryl), optionally substituted —O(heterocyclyl), optionally substituted —O(heteroaryl), a phosphonate group, and a protected alcohol. When the substituent is further substituted, it may be substituted with 1, 2, or 3 groups independently chosen from -halo, —NO₂, —CN, -alkyl, -alkoxy, -cycloalkyl, -aryl, -heterocyclyl, and -heteroaryl.

In some embodiments, a heterocyclyl group is optionally substituted with 1, 2, or 3 groups independently chosen from -halo, —OH, —CN, —NO₂, optionally substituted alkyl, optionally substituted alkoxy, —C(O)alkyl, —SO₂(alkyl), optionally substituted aryl, —O(aryl), -amido, -phosphonate group, and a protected alcohol.

The term “hydroxy” or “hydroxyl” refers to a group —OH.

The term “oxo” refers to a ═O substituent.

The term “phosphonate group” refers to a —P(O)—(OR)₂, —O—P(O)—(OR)₂, —P(O)(OR)(R), or —OP(O)(OR)(R) group, or a string of such groups, wherein each R group are independently chosen from H, an optionally substituted alkyl, and an optionally substituted aryl. For example, in some embodiments, the phosphonate group is chosen from —O—P(O)—(OH)₂, —P(O)—(OCH₂Phenyl)₂,

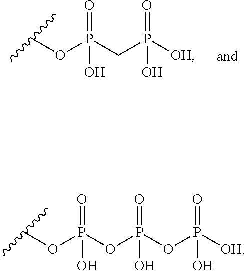 [SMILES: *OP(=O)(O)CP(=O)(O)O ; *OP(=O)(O)OP(=O)(O)OP(=O)(O)O]

The term “protected alcohol” would be understood by those of ordinary skill in the chemical arts and includes, for example, benzyl ether (—OCH₂Phenyl), DMT (dimethoxytrityl), silyl ethers such as TMS (trimethylsilyl), THP (tetrahydropyranyl), PMB (p-methoxybenzyl) and BOC (ter-butyloxycarbonyl). In some embodiments, the protected alcohol is benzyl ether (—OCH₂Phenyl) or DMT (dimethoxytrityl).

The term “intermediate” or “intermediate compound” refers to a compound which is produced in the course of a chemical synthesis, which is not itself the final product, but is used in further reactions which produce the final product. There may be many different intermediate compounds between the starting material and end product in the course of a complex synthesis.

The term “about” indicates and encompasses an indicated value and a range above and below that value. In certain embodiments, the term “about” indicates the designated value 10%, +5%, or +1%. In certain embodiments, where applicable, the term “about” indicates the designated value(s)±one standard deviation of that value(s).

The term “administration”, or a variant thereof (e.g. “administering”), means providing the active agent or active ingredient, alone or as part of a pharmaceutically acceptable composition, to the patient in whom/which the condition, symptom, or disease is to be treated or prevented.

The term “antagonist” refers to a natural or synthetic compound which binds to the protein and blocks the biological activation of the protein, and thereby the action of the said protein. The protein may be a receptor, i.e. a protein molecule that receives chemical signals from outside a cell. Consequently, “an adenosine receptor antagonist” includes any chemical entity that, upon administration to a patient, results in inhibition or down-regulation of a biological activity associated with activation of an adenosine receptor in the patient, including any of the downstream biological effects otherwise resulting from the binding to an adenosine receptor of its natural ligand. Such adenosine receptor antagonists include any agent that can block activation of an adenosine receptor or any of the downstream biological effects of an adenosine receptor activation.

The term “adenosine receptor antagonist” refers to a compound that, upon administration to a patient, results in inhibition or down-regulation of a biological activity associated with activation of an adenosine receptor in the patient, including any of the downstream biological effects otherwise resulting from the binding to an adenosine receptor of its natural ligand. Such adenosine receptor antagonists include any agent that can block activation of an adenosine receptor or any of the downstream biological effects of an adenosine receptor activation.

The term “inhibitor” refers to a natural or synthetic compound that has a biological effect to inhibit or significantly reduce or down-regulate the expression of a gene and/or a protein. Consequently, an “ENT inhibitor” or “inhibitor of an ENT family transporter” refers to a compound that has a biological effect to inhibit or significantly reduce or down-regulate the biological activity of ENT family transporter. In one embodiment, the compounds of the present disclosure (e.g., compounds of Formula (I)) are ENT inhibitors, for example, ENT1 inhibitors.

The term “chemotherapy” refers to a type of cancer treatment that uses one or more anti-cancer drugs (chemotherapeutic agents) as part of a standardized chemotherapy regimen. Chemotherapy may be given with a curative intent or it may aim to prolong life or to reduce symptoms. Chemotherapeutic agents include, for example, anticancer alkylating agents, anticancer antimetabolites, anticancer antibiotics, plant-derived anticancer agents, anticancer platinum coordination compounds, and any combination thereof.

The phrases “compounds disclosed herein,” “compounds of the present disclosure” and the like encompass all compounds within the genuses of Formula (I), (Ia), (Ib), (Ic), (Id), and (Ie) as well as all compounds disclosed in Table 1 and the Examples. The phrases also include pharmaceutical salts, hydrates, and solvates thereof, whether or not salts, hydrates, and solvates are explicitly recited. The phrase also includes deuterated forms of any of the compounds of the present disclosure, whether or not deuderated is explicitly recited. As used herein, “deuterated”, means a compound wherein at least one hydrogen (H) has been replaced by deuterium (indicated by D or ²H), that is, the compound is enriched in deuterium in at least one position.

The term “hormone therapy” refers to the use of hormones in medical treatment. In one embodiment, the hormone therapy is oncologic hormone therapy.

The term “patient” refers to a mammal, such as a human, who/which is awaiting the receipt of, or is receiving medical care, or was/is/will be the object of a medical procedure, or is monitored for the development or progression of a disease, such as a cancer.

The term “immunotherapy” refers to a therapy aiming at inducing and/or enhancing an immune response towards a specific target, for example towards cancer cells. Immunotherapy may involve the use of checkpoint inhibitors, checkpoint agonists (also called T-cell agonists), IDO inhibitors, PBK inhibitors, adenosine receptor inhibitors, adenosine-producing enzymes inhibitors, adoptive transfer, and combinations thereof.

The expression “pharmaceutically acceptable” refers to compounds, salts, hydrates, solvates, compositions, dosage forms, and other materials which are useful in preparing a pharmaceutical composition that is suitable for veterinary or human pharmaceutical use.

The expression “pharmaceutically acceptable carrier” or “pharmaceutically acceptable excipient” or “excipient” includes any and all solvents, dispersion media, coatings, antibacterial and antifungal agents, isotonic and absorption delaying agents and the like. The use of such media and agents for pharmaceutically active substances is known in the art. Except insofar as any conventional media or agent is incompatible with the active ingredient, its use in the therapeutic compositions is contemplated. Supplementary active ingredients can also be incorporated into the compositions.

The terms “prevent”, “preventing” and “prevention”, as used herein, refer to delaying or precluding the onset of a condition or disease and/or its attendant symptoms, barring a patient from acquiring a condition or disease, or reducing a patient's risk of acquiring a condition or disease.

The term “prodrug” as used herein refers to pharmacologically acceptable derivatives of any of the compounds disclosed herein, for example compounds of Formula (I) and in Table 1, whose in vivo biotransformation product generates the biologically active drug. Prodrugs can be, for example, esters or amides.

The term “radiation therapy” refers to a method of treatment of cancer employing various radiations such as X-ray, gamma-ray, neutron ray, electron beam, proton beam and radiation sources. It is used as part of cancer treatment to control or kill malignant cells. Radiation therapy may be curative in a number of types of cancer if they are localized to one area of the body. It may also be used as part of adjuvant therapy, to prevent tumor recurrence after surgery to remove a primary malignant tumor. The three main divisions of radiation therapy are: external beam radiation therapy (EBRT or XRT); brachytherapy or sealed source radiation therapy; and systemic radioisotope therapy (RIT) or unsealed source radiotherapy.

The terms “therapeutically effective amount” or “effective amount” or “therapeutically effective dose” or dose of a compound or a composition refer to that amount of the compound or the composition that results in reduction or inhibition of symptoms or a prolongation of survival in a subject (such as a human patient). The results may require multiple doses of the compound or the composition. A therapeutically effective amount may be administered prior to the onset of a disease or disorder for a prophylactic or preventive action. Alternatively, or additionally, a therapeutically effective amount may be administered after initiation of a disease or disorder for a therapeutic action. In one embodiment, the disease or disorder is cancer.

The term “stem cell transplant” refers to a procedure in which a patient receives healthy bloodforming cells (stem cells) to replace their own that have been destroyed by disease or by the radiation or high doses of anticancer drugs that are given as part of the procedure. The healthy stem cells may come from the blood or bone marrow of the patient, from a donor, or from the umbilical cord blood of a newborn baby. A stem cell transplant may be autologous (using a patient's own stem cells that were collected and saved before treatment), allogeneic (using stem cells donated by someone who is not an identical twin), or syngeneic (using stem cells donated by an identical twin).

The term “subject” refers to a mammal, for example a human. In one embodiment, the subject is diagnosed with a cancer. In one embodiment, the subject is a patient, for example a human patient, who/which is awaiting the receipt of, or is receiving medical care, or was/is/will be the subject of a medical procedure, or is monitored for the development or progression of a disease, such as a cancer. In one embodiment, the subject is a human patient who is being treated and/or monitored for the development or progression of a cancer. In one embodiment, the subject is a male. In another embodiment, the subject is a female. In one embodiment, the subject is an adult. In another embodiment, the subject is a child.

Although various features of the present disclosure may be described in the context of a single embodiment, the features may also be provided separately or in any suitable combination.

### Compounds

In one aspect, provided herein is a compound of Formula (I):

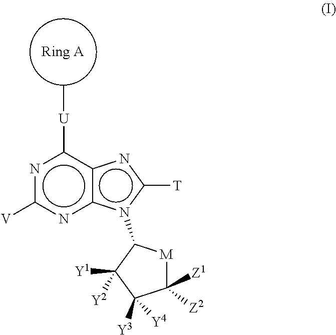

or a pharmaceutically acceptable salt, hydrate, or solvate thereof, wherein:
Ring A is chosen from optionally substituted aryl, optionally substituted heteroaryl containing at least one N, O, or S atom, optionally substituted heterocyclyl containing at least one N or O, and optionally substituted cycloalkyl;
U is a direct bond or is chosen from: (i) —O—, (ii) -alkoxy-, (iii) -(alkyl)O(alkyl)-, (iv) -alkyl-, (v) -alkenyl-, (vi) -alkyl-S— wherein alkyl is attached to Ring A, (vii) —SO₂NR¹— wherein the N is attached to Ring A, (viii) -alkyl-SO₂—NR¹— wherein the N is attached to Ring A, (ix) -alkyl-NR¹— wherein alkyl is attached to Ring A, (x) —NR¹—, (xi) —C(O)NR¹— wherein the N is attached to Ring A, (xii) —C(O)NR¹-alkyl- wherein the alkyl is attached to Ring A, and (xiii) —CO—;
   T is chosen from —H, —OH, —O(alkyl)(aryl), optionally substituted heteroaryl that contains at least one N atom, —C(O)NR¹(cycloalkyl), and optionally substituted amine;
   V is chosen from —H, -halo, —OH, -alkyl, and -alkoxy;
   M is —O— or —C(R²)₂—;
   Y¹ and Y² are each independently chosen from —H, —OH, and -halo;
   Y³ is —OH or —H;
   Y⁴ is chosen from —H, -halo, —OH, and -alkyl optionally substituted with 1, 2, or 3 groups independently chosen from -halo and —OH;
   Z¹ is chosen from —H, -halo, —OH, and -alkyl optionally substituted with 1, 2, or 3 groups independently chosen from —OH and -halo;
   Z² is chosen from —H, -alkyl, -alkenyl, —C(O)NHR¹, —C(O)NR¹(alkyl), and —C(O)O(alkyl), wherein each alkyl and alkenyl group is optionally substituted;
   each R¹ is independently chosen from —H and -alkyl; and
   each R² is independently chosen from —H, -alkyl, and -cycloalkyl;
on the condition that:
(a) when U is chosen from (i) —O—, (ii) -alkoxy-, and (iii) -(alkyl)O(alkyl)-, then:
   at least one of Y¹, Y² or Y³ is —OH; and
   when Z² is alkyl, it is not substituted with a phosphonate group or a protected alcohol group; and
with the proviso that the compound is not

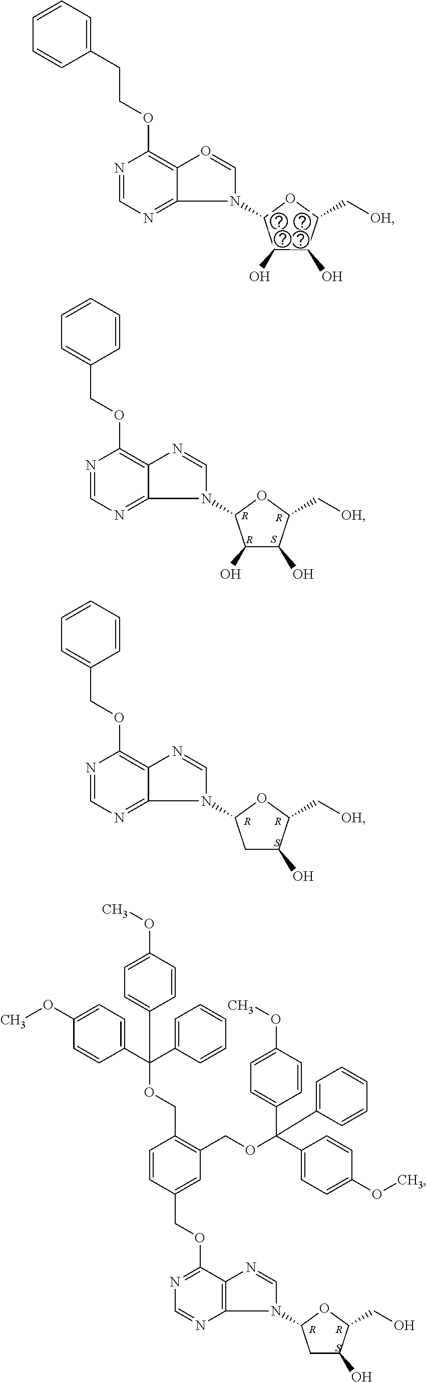

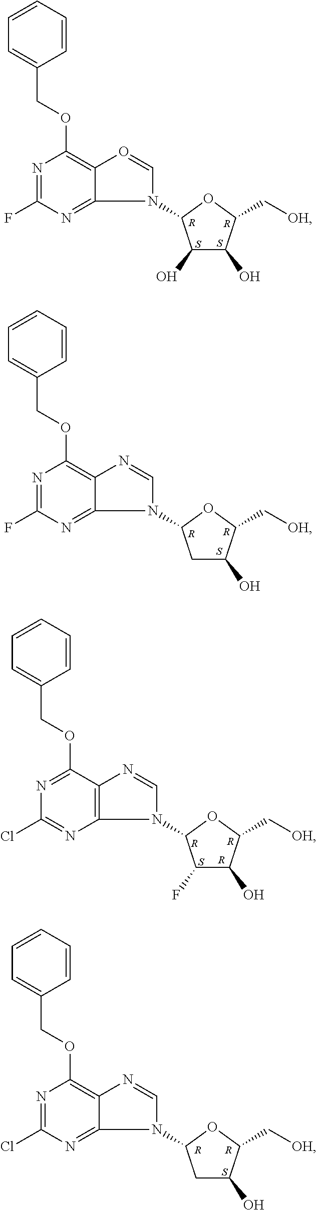

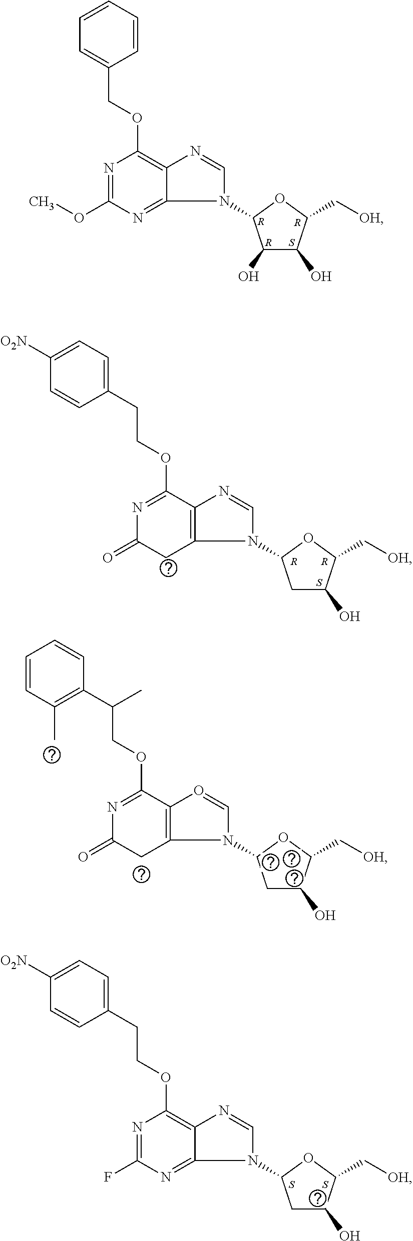

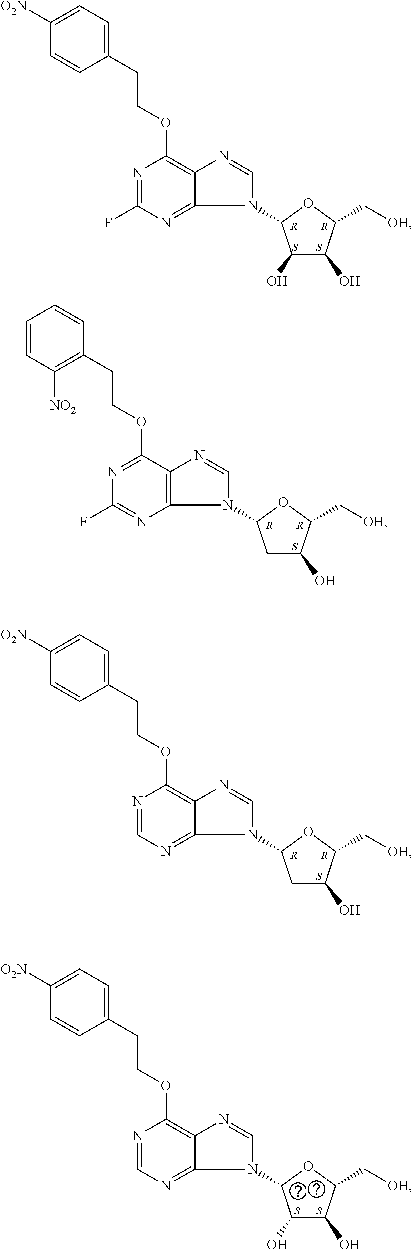

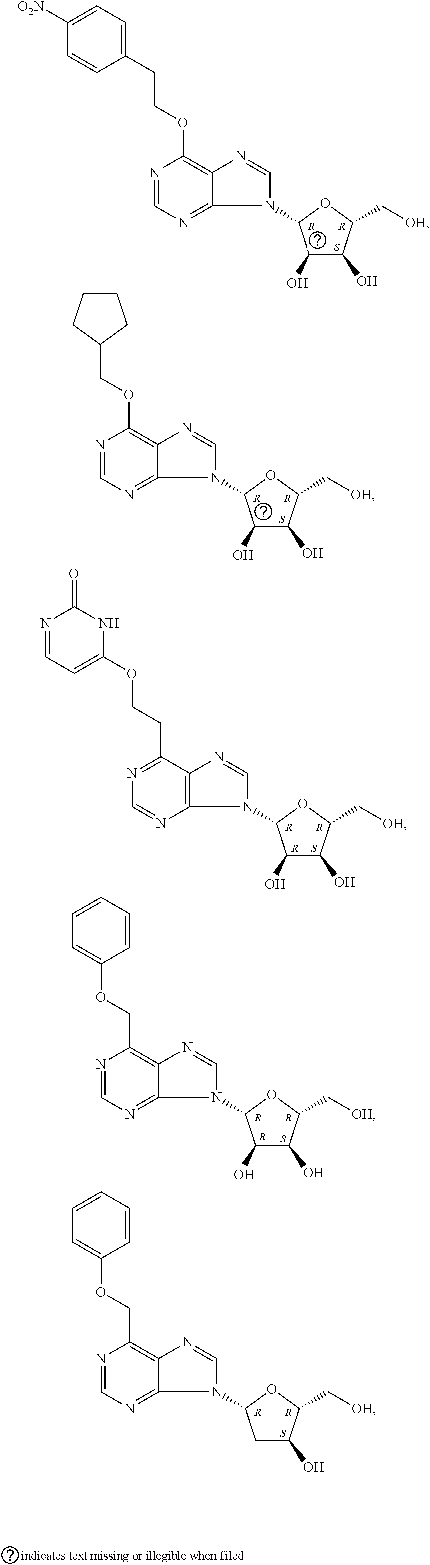

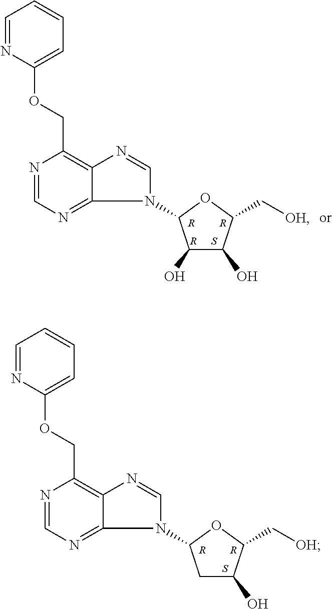

further on the condition that:
(b) when U is chosen from (iv) -alkyl- and (v) -alkenyl-, then:
   Ring A is an optionally substituted aryl; and
   at least one of Y¹, Y² or Y³ is —OH; and
   Z² is -alkyl; and
further on the condition that:
(c) when U is chosen from (ix) -alkyl-NR¹— wherein alkyl is attached to Ring A, (x) —NR¹—, (xiii) —CO—, (xi) —C(O)NR¹— wherein the N is attached to Ring A, and (xii) —C(O)NR¹-alkyl- wherein the alkyl is attached to Ring A; then:
   Ring A an optionally substituted aryl; and
   M is —O— or —CH₂—; and
   Z² is -alkyl; and
   each R¹ is independently chosen from —H and -alkyl; and
   with the proviso that the compound is not

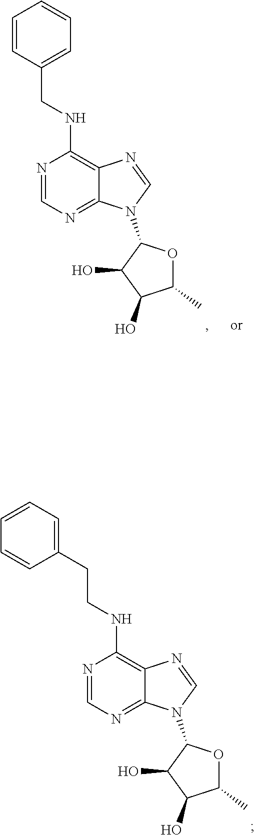 [SMILES: C[C@H]1O[C@@H](n2cnc3c(NCc4ccccc4)ncnc32)[C@H](O)[C@@H]1O ; C[C@H]1O[C@@H](n2cnc3c(NCCc4ccccc4)ncnc32)[C@H](O)[C@@H]1O]

further on the condition that:
(d) when U is chosen from: (vi) -alkyl-S— wherein alkyl is attached to Ring A, (vii) —SO₂NR¹— wherein the N is attached to Ring A, and (viii) -alkyl-SO₂—NR¹— wherein the N is attached to Ring A; then:
   Ring A is C₆ aryl substituted with 1 or 2 groups independently chosen from —NO₂, —CN, —CH₂CN, alkoxy optionally substituted with 1, 2, or 3-halo atoms, and 5 membered heteroaryl optionally substituted with 1, 2, or 3 —CH₃ groups; and
   T is —H; and
with the proviso that the compound is not

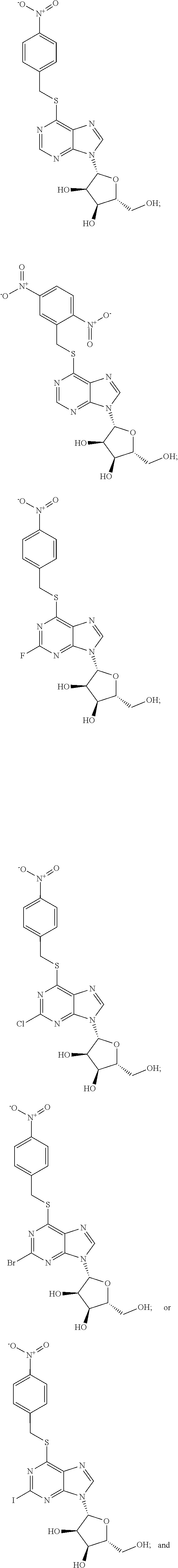

further on the condition that:
(e) when U is a direct bond, then
   M is —O— or —CH₂—; and
   Z² is -alkyl; and
   R¹ is —H or -alkyl.

In some embodiments, V is chosen from —H, -halo, —OH, -alkyl, and -alkoxy. In some embodiments, V is chosen from —H, -halo, —OH, —C₁₋₆ alkyl, and —C₁₋₆ alkoxy. In some embodiments, V is chosen from —H and -halo. In some embodiments, V is —H. In some embodiments, V is -halo. In some embodiments, V is chosen from —F, —Cl, and Br. In some embodiments, V is —F. In some embodiments, V is —OH. In some embodiments, V is —C₁₋₆ alkyl, such as —CH₃. In some embodiments, V is —C₁₋₆ alkoxy, such as —OCH₃.

In some embodiments, M is —O— or —C(R²)₂—. In some embodiments, M is —O— or —CH₂—. In some embodiments, M is —O—.

In some embodiments, Y¹ and Y² are each independently chosen from —H, —OH, and -halo. In some embodiments, Y¹ and Y² are each independently chosen from —H, —OH, —F, —Cl, and —Br. In some embodiments, Y¹ and Y² are each independently chosen from —H, —OH, and —F.

In some embodiments, Y³ is —OH or —H. In some embodiments, Y³ is —OH. In some embodiments, Y³ is —H.

In some embodiments, Y⁴ is chosen from —H, -halo, —OH, and alkyl optionally substituted with 1, 2, or 3 groups independently chosen from -halo and —OH. In some embodiments, Y⁴ is chosen from —H, —F, —Cl, —Br, —OH, and C₁₋₆ alkyl optionally substituted with 1, 2, or 3 groups independently chosen from —F, —Cl, —Br, and —OH. In some embodiments, Y⁴ is chosen from —H, —F, —Cl, and C₁₋₆ alkyl optionally substituted with 1, 2, or 3 groups independently chosen from —F and —Cl. In some embodiments, Y⁴ is chosen from —H and —F. In some embodiments, Y⁴ is —H.

In some embodiments, Y¹ is —H and Y² is —OH. In some embodiments, Y¹ is —OH and Y² is —H. In some embodiments, Y¹ is —F and Y² is —OH. In some embodiments, Y¹ is —F and Y² is —H. In some embodiments, both Y¹ and Y² are —H. In some embodiments, both Y¹ and Y² are —F.

In some embodiments, Y³ is —H and Y⁴ is —OH. In some embodiments, Y³ is —OH and Y⁴ is —H. In some embodiments, Y³ is —F and Y⁴ is —OH. In some embodiments, Y³ is —OH and Y⁴ is —F.

In some embodiments, Y¹ and Y³ are both —OH. In some embodiments, Y¹ is —OH and Y³ is —H. In some embodiments, Y¹ is —H and Y³ is —OH.

In some embodiments, Y² and Y⁴ are both —H. In some embodiments, Y² is —OH and Y⁴ is —H. In some embodiments, Y² is —OH and Y⁴ is —H.

In some embodiments, Z¹ is chosen from —H, -halo, —OH, and -alkyl optionally substituted with 1, 2, or 3 groups independently chosen from —OH and -halo. In some embodiments, Z¹ is chosen from —H, -halo, —OH, and —C₁₋₆ alkyl optionally substituted with 1, 2, or 3 groups independently chosen from —OH and -halo. In some embodiments, Z¹ is chosen from —H, -halo, —OH, and —C₁₋₆ alkyl. In some embodiments, Z¹ is chosen from —H, —F, and —CH₃. In some embodiments, Z¹ is —H. In some embodiments, Z¹ is —F. In some embodiments, Z¹ is —CH₃.

In some embodiments, each R¹ is independently chosen from —H and -alkyl. In some embodiments, each R¹ is independently chosen from —H and —C₁₋₆ alkyl. In some embodiments, each R¹ is —C₁₋₆ alkyl. In some embodiments, each R¹ is —H. In some embodiments, each R¹ is —CH₃.

In some embodiments, each R² is independently chosen from —H, -alkyl, and -cycloalkyl. In some embodiments, each R² is independently chosen from —H, —C₁₋₆ alkyl, and —C₃₋₁₀ cycloalkyl. In some embodiments, each R² is independently chosen from —H, —C₁₋₆ alkyl, and —C₃₋₆ cycloalkyl. In some embodiments, each R² is independently chosen from —H and —C₁₋₆ alkyl. In some embodiments, each R² is —H. In some embodiments, each R² is —CH₃. In some embodiments, one R² is —H and one R² is —CH₃.

In one aspect, provided herein is a compound of Formula (Ia):

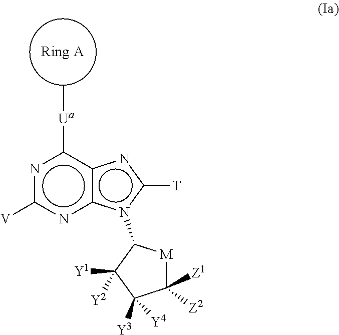

or a pharmaceutically acceptable salt, hydrate, or solvate thereof, wherein:
   Ring A is chosen from optionally substituted aryl, optionally substituted heteroaryl containing at least one N, O, or S atom, optionally substituted heterocyclyl containing at least one N or O, and optionally substituted cycloalkyl;
   U^{a} is chosen from: (i) —O—, (ii) -alkoxy-, and (iii) -(alkyl)O(alkyl)-;
   T is chosen from —H, —OH, —O(alkyl)(aryl), optionally substituted heteroaryl that contains at least one N atom, —C(O)NR¹(cycloalkyl), and optionally substituted amine;
   V is chosen from —H, -halo, —OH, -alkyl, and -alkoxy;
   M is —O— or —C(R²)₂—;
   Y¹ and Y² are each independently chosen from —H, —OH, and -halo;
      wherein at least one of Y¹, Y² or Y³ must be —OH;
   Y³ is —OH or —H;
   Y⁴ is chosen from —H, -halo, —OH, and alkyl optionally substituted with 1, 2, or 3 groups independently chosen from -halo and —OH;
   Z¹ is chosen from —H, -halo, —OH, and alkyl optionally substituted with 1, 2, or 3 groups independently chosen from —OH and -halo;
   Z² is chosen from —H, -alkyl, -alkenyl, —C(O)NHR¹, —C(O)NR¹(alkyl), and —C(O)O(alkyl), wherein each alkyl and alkenyl group is optionally substituted;
   each R¹ is independently chosen from —H and -alkyl; and
   each R² is independently chosen from —H, -alkyl, and -cycloalkyl;
wherein when Z² is alkyl, it is not substituted with a phosphonate group or a protected alcohol group; and
with the proviso that the compound is not

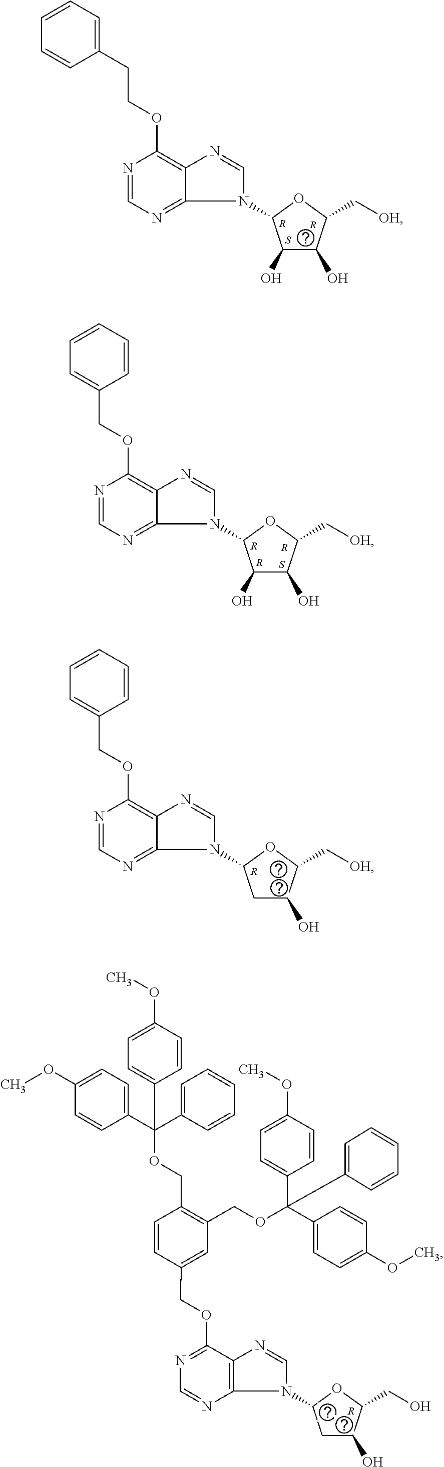

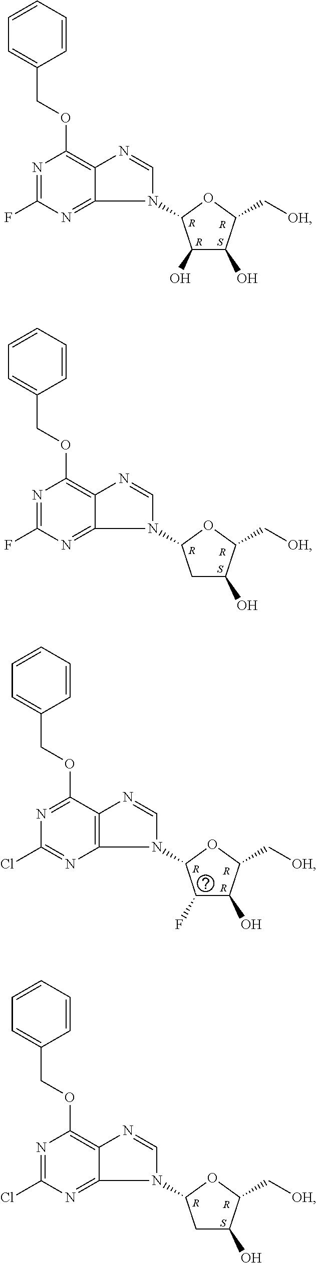

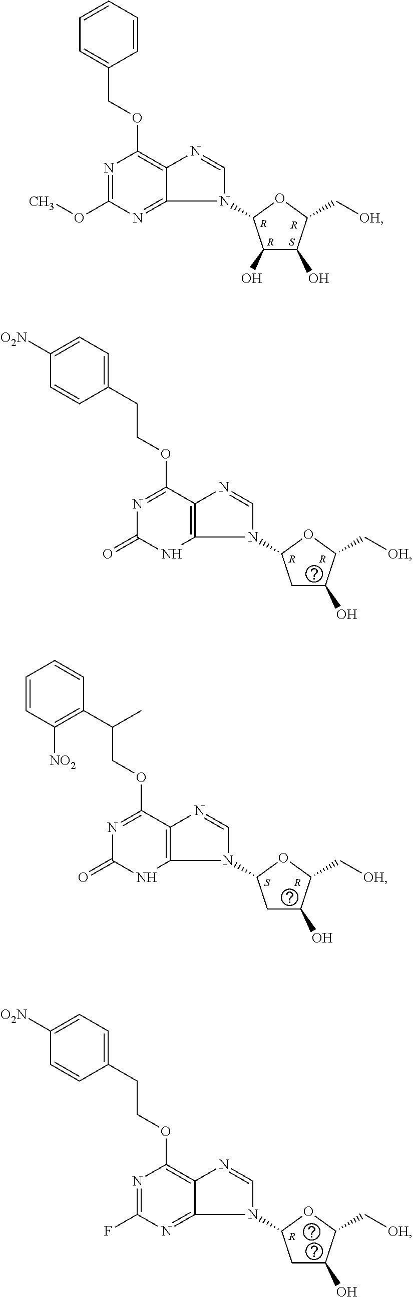

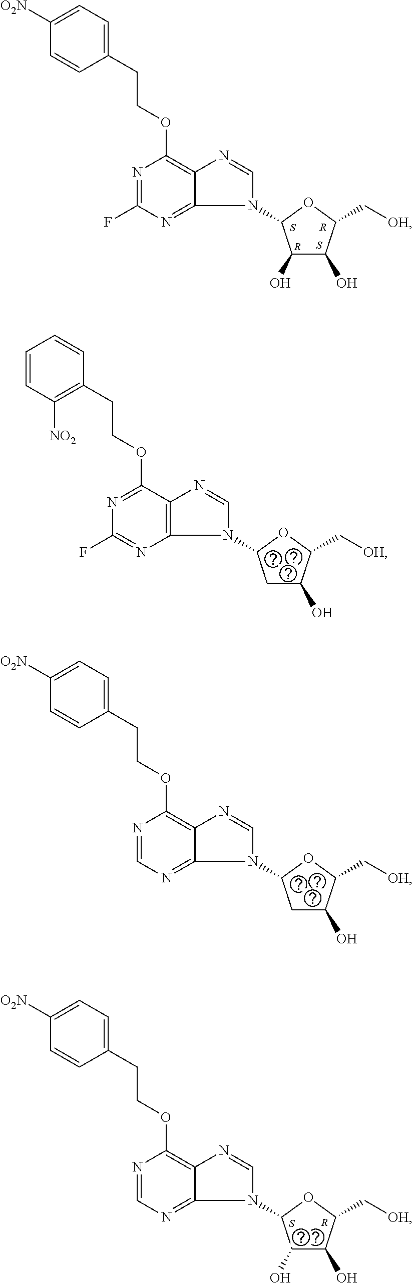

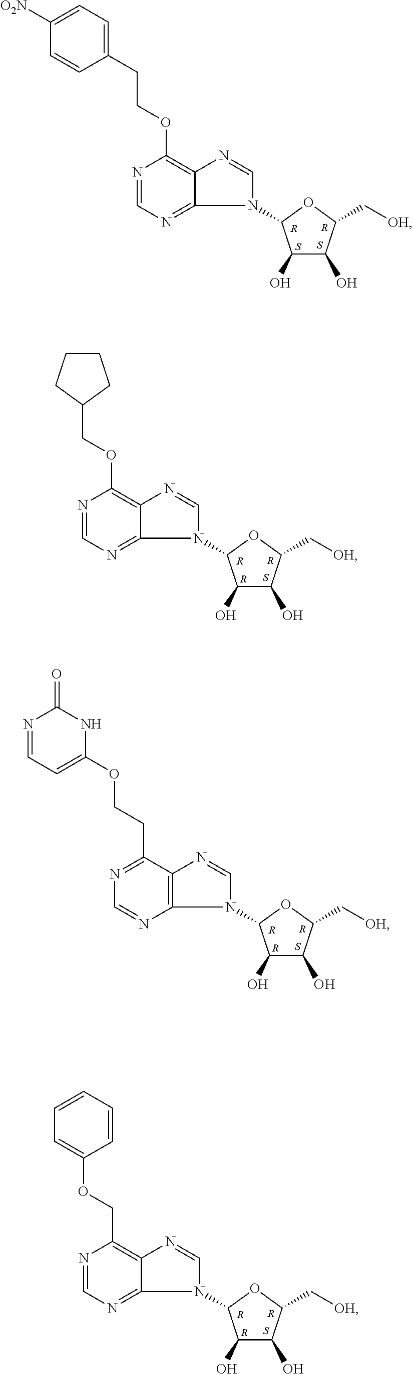

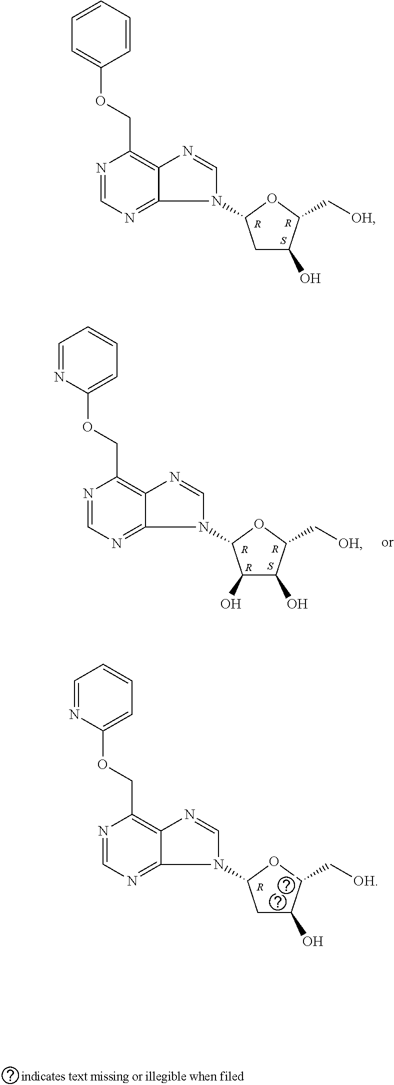

In some embodiments, Ring A is chosen from optionally substituted aryl, optionally substituted heteroaryl containing at least one N, O, or S atom, optionally substituted heterocyclyl containing at least one N or O, and optionally substituted cycloalkyl. In some embodiments, Ring A is chosen from optionally substituted C₆₋₁₂ aryl, optionally substituted 5-12 membered heteroaryl containing at least one N, O, or S atom, optionally substituted 3-11 membered heterocyclyl containing at least one N or O, and optionally substituted C₃₋₁₀ cycloalkyl.

In some embodiments, Ring A is chosen from an optionally substituted C₆ aryl and an optionally substituted 5-6 membered heteroaryl. In some embodiments, Ring A is chosen from an optionally substituted C₆ aryl and an optionally substituted 6 membered heteroaryl.

In some embodiments, Ring A is chosen from cyclopenta-1,3-dienyl, phenyl, pyrrolyl, imidazolyl, pyrazolyl, triazolyl, furanyl, thiophenyl, oxazolyl, thiazolyl, pyridinyl, pyrimidinyl, pyrazinyl, and pyridazinyl, each of which is optionally substituted. In some embodiments, Ring A is chosen from phenyl, pyridinyl, and pyridazinyl, each of which is optionally substituted.

In some embodiments, each optional substituent of Ring A is independently chosen from: (i) —NO₂, (ii) —C₁₋₆ alkyl optionally substituted with 1, 2, or 3 —F atoms, (iii) —O(C₁₋₆ alkyl) wherein the alkyl is optionally substituted with 1, 2, or 3 —F atoms, (iv) -halo, and (v) —C₃₋₆ cycloalkyl. In some embodiments, each optional substituent of Ring A is independently chosen from —NO₂, —CH₃, —OCHF₂, —OCF₃, —OCH₂CF₃, —CN, —F, —Cl, —CHF₂, —CF₃, and -cyclopropyl. In some embodiments, each optional substituent of Ring A is independently chosen from —NO₂, —CH₃, —OCHF₂, —OCF₃, —OCH₂CF₃, —CN, —F, —Cl, —CHF₂, and —CF₃.

In some embodiments, Ring A is phenyl optionally substituted with 1, 2, or 3 groups independently chosen from —NO₂, —CH₃, —OCHF₂, —OCF₃, —OCH₂CF₃, —CN, —F, —Cl, —CHF₂, —CF₃, and -cyclopropyl. In some embodiments, Ring A is pyridinyl optionally substituted with 1, 2, or 3 groups independently chosen from —NO₂, —CH₃, —OCHF₂, —OCF₃, —OCH₂CF₃, —CN, —F, —Cl, —CHF₂, —CF₃, and -cyclopropyl.

In some embodiments, U^{a} is chosen from: (i) —O—, (ii) -alkoxy-, and (iii) -(alkyl)O(alkyl)-. In some embodiments, U^{a} is —O—.

In some embodiments, U^{a} is -alkoxy-. In some embodiments, U^{a} is a C₁₋₆ alkoxy group. In some embodiments, U^{a} is -alkoxy- wherein the alkyl group of the alkoxy group is attached to Ring A. In some embodiments, U^{a} is a C₁₋₆ alkoxy group wherein the alkyl group of the alkoxy group is attached to Ring A. In some embodiments, U^{a} is —CH₂CH₂O— wherein the —CH₂CH₂— is attached to Ring A. In some embodiments, U^{a} is —CH₂O— wherein the —CH₂— is attached to Ring A.

In some embodiments, U^{a} is -(alkyl)O(alkyl)-. In some embodiments, U^{a} is —(C₁₋₆ alkyl)O(C₁₋₆ alkyl)-. In some embodiments, U^{a} is —(C₁₋₂ alkyl)O(C₁₋₂ alkyl)-.

In some embodiments, U^{a} is -alkoxy- and Ring A is an optionally substituted aryl or an optionally substituted heteroaryl containing at least one N, O, or S atom. In some embodiments, U^{a} is -alkoxy- and Ring A is an optionally substituted pyridinyl or an optionally substituted phenyl. In some embodiments, U^{a} is -alkoxy- and Ring A is pyridinyl or a phenyl optionally substituted with 1, 2, or 3 groups independently chosen from —OCH₂CF₃, —CN, —F.

In some embodiments, T is chosen from —H, —OH, —O(alkyl)(aryl), optionally substituted heteroaryl that contains at least one N atom, —C(O)NR¹(cycloalkyl), and optionally substituted amine. In some embodiments, T is chosen from —H, —OH, —O(C₁₋₆ alkyl)(C₆₋₁₀ aryl), optionally substituted 5-12 membered heteroaryl that contains at least one N atom, —C(O)NR¹(C₃₋₁₀ cycloalkyl), and optionally substituted amine. In some embodiments, T is chosen from —H, —OH, —O(C₁₋₄ alkyl)(C₆ aryl), optionally substituted 5-6 membered heteroaryl that contains at least one N atom, —C(O)NR¹(C₃₋₆ cycloalkyl), and optionally substituted amine.

In some embodiments, T is chosen from —H, —O(CH₂)(C₆ aryl), —C(O)NR¹(C₃₋₅ cycloalkyl), optionally substituted 5 membered heteroaryl, and an optionally substituted amine.

In some embodiments, T is chosen from —H, —O(CH₂)(C₆ aryl), —C(O)NH(C₃₋₅ cycloalkyl), and optionally substituted pyrazolyl. In some embodiments, T is chosen from —H,

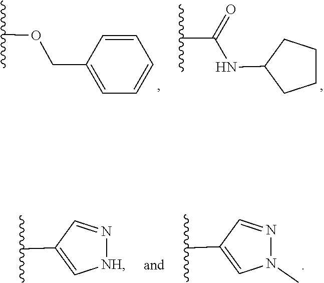

In some embodiments, T is an optionally substituted amine. In some embodiments, T is an optionally substituted amine chosen from —N(R¹)₂, —NR¹(C₁₋₄ alkyl), —NR¹(C₃₋₆ cycloalkyl), —NR¹(CH₂)₁₋₂(C₃₋₆ cycloalkyl), —NR¹(5-6 membered heterocyclyl), —NR¹(CH₂)₁₋₂(5-6 membered heterocyclyl), —NR₁(5-6 membered heteroaryl), —NR¹(CH₂)₁₋₂(5-6 membered heteroaryl), —NR¹(C₆ aryl) and —NR¹(CH₂)₁₋₂(C₆ aryl), wherein each alkyl, cycloalkyl, heterocyclyl, heteroaryl, or aryl is optionally substituted.

In some embodiments, T is an optionally substituted amine chosen from —N(R¹)₂, —NR¹(C₁₋₄ alkyl), —NR¹(C₃₋₆ cycloalkyl), —NR¹(CH₂)₁₋₂(C₃₋₆ cycloalkyl), —NR¹(5-6 membered heterocyclyl), —NR¹(CH₂)₁₋₂(5-6 membered heterocyclyl), —NR¹(5-6 membered heteroaryl), —NR¹(CH₂)₁₋₂(5-6 membered heteroaryl), —NR¹(C₆ aryl) and —NR¹(CH₂)₁₋₂(C₆ aryl), wherein each alkyl, cycloalkyl, heterocyclyl, heteroaryl, or aryl is optionally substituted with 1, 2, or 3 groups independently chosen from —OH, -halo, —CN, —CH₃, —C(O)CH₃, —OCH₃, —SO₂CH₃, and —O—(C₆ aryl).

In some embodiments, T is an optionally substituted amine chosen from:
(i) —N(R¹)₂,
(ii) —NR¹(C₁₋₆ alkyl) wherein the alkyl is optionally substituted with 1, 2, or 3 groups independently chosen from -halo, —OH, —SO₂CH₃, —OCH₃, and —O(C₆ aryl),
(iii) —NR¹(C₃₋₆ cycloalkyl) wherein the cycloalkyl is optionally substituted with 1, 2, or 3 groups independently chosen from —CH₃, -halo, and —OH,
(iv) —NR¹CH₂(C₃₋₆ cycloalkyl) wherein the cycloalkyl is optionally substituted with 1, 2, or 3 groups independently chosen from —CH₃, -halo, and —OH,
(v) —NR¹(5-6 membered heterocyclyl) wherein the heterocyclyl is optionally substituted with 1, 2, or 3 groups independently chosen from —C(O)CH₃, —CH₃, -halo, and —OH,
(iv) —NR¹CH₂(5-6 membered heterocyclyl) wherein the heterocyclyl is optionally substituted with 1, 2, or 3 groups independently chosen from —C(O)CH₃, —CH₃, -halo, and —OH,
(v) —NR¹CH₂CH₂ (5-6 membered heterocyclyl) wherein the heterocyclyl is optionally substituted with 1, 2, or 3 groups independently chosen from —C(O)CH₃, —CH₃, -halo, and —OH,
(vi) —NR¹(5-6 membered heteroaryl) wherein the heteroaryl is optionally substituted with 1, 2, or 3 groups independently chosen from —CH₃, —CN, and —OCH₃,
(vii) —NR¹(CH₂)₁₋₂(5-6 membered heteroaryl) wherein the heteroaryl is optionally substituted with 1, 2, or 3 groups independently chosen from —CH₃, —CN, and —OCH₃,
(viii) —NR¹(C₆ aryl) wherein the aryl is optionally substituted with 1, 2, or 3 groups independently chosen from —CH₃, —CN, and —OCH₃,
(ix) —NR¹(CH₂)₁₋₂(C₆ aryl) wherein the aryl is optionally substituted with 1, 2, or 3 groups independently chosen from —CH₃, —CN, and —OCH₃.

In some embodiments, T is an optionally substituted amine chosen from:

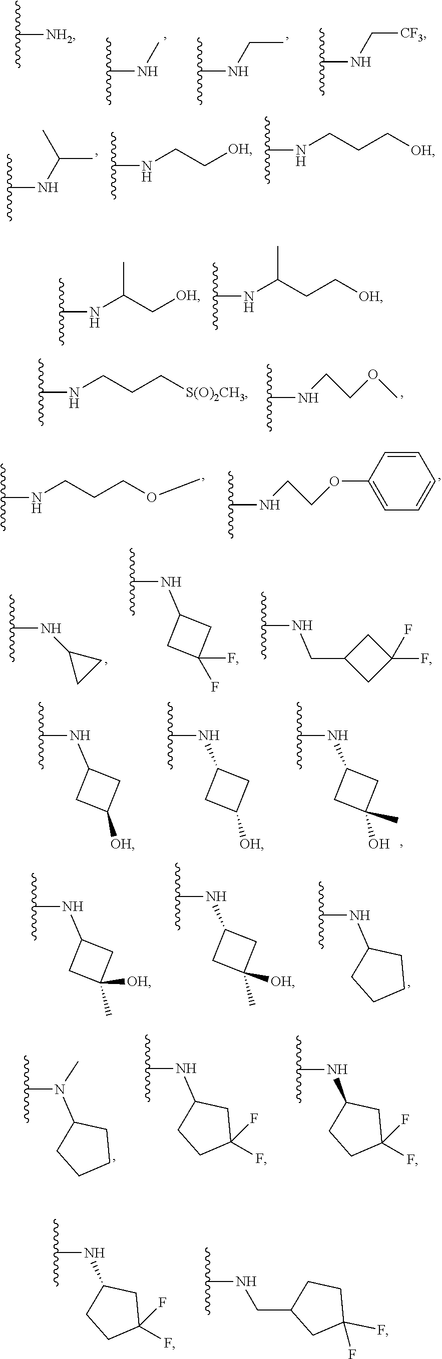

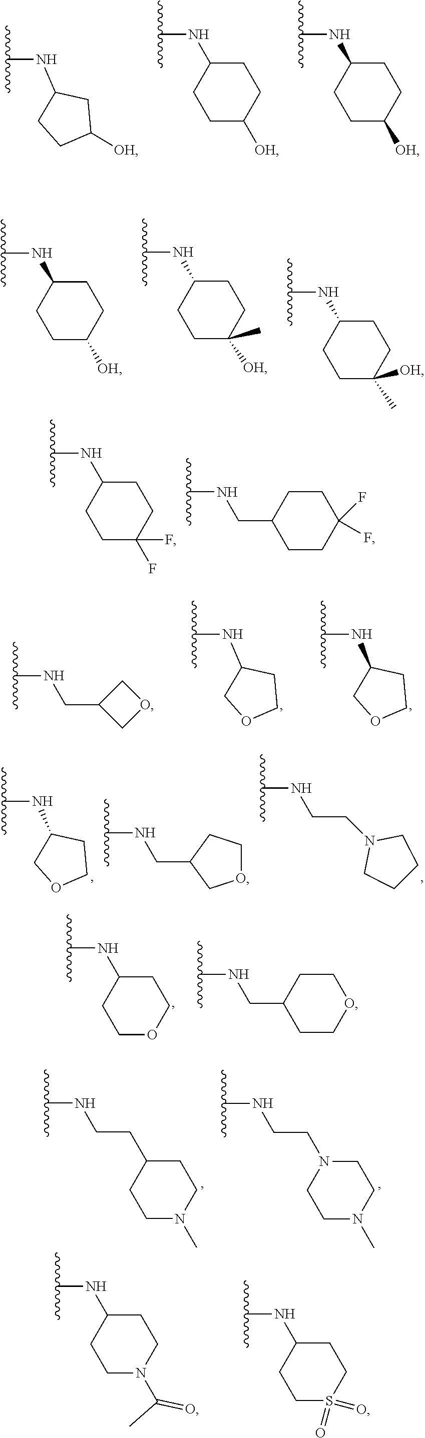

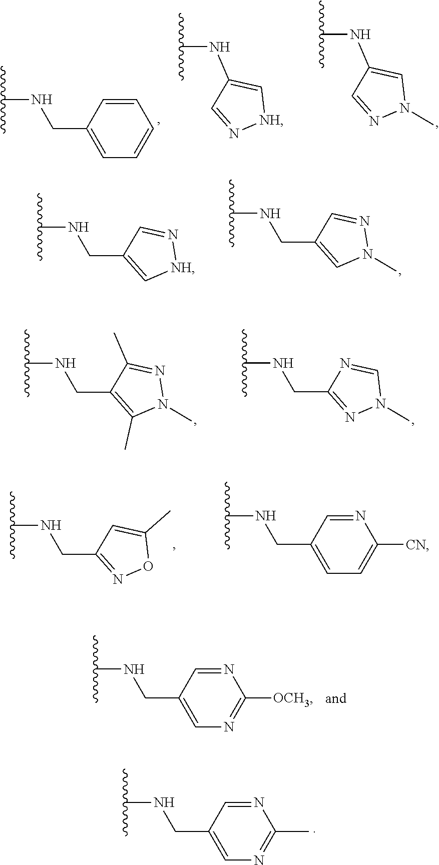

In some embodiments, T is an optionally substituted amine chosen from:

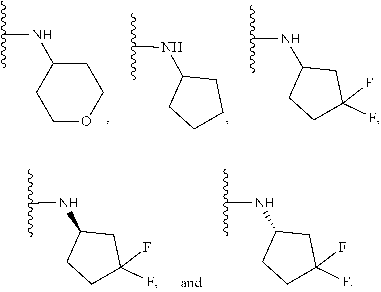 [SMILES: *NC1CCOCC1 ; *NC1CCCC1 ; *NC1CCC(F)(F)C1 ; *N[C@@H]1CCC(F)(F)C1 ; *N[C@H]1CCC(F)(F)C1]

In some embodiments, T is

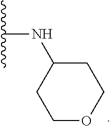 [SMILES: *NC1CCOCC1]

In some embodiments, T is

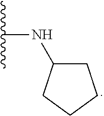 [SMILES: *NC1CCCC1]

In some embodiments, T is

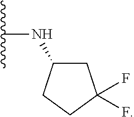 [SMILES: *N[C@H]1CCC(F)(F)C1]

In some embodiments, U^{a} is -alkoxy-, Ring A is an optionally substituted aryl or an optionally substituted heteroaryl containing at least one N, O, or S atom, and T is an optionally substituted amine. In some embodiments, U^{a} is -alkoxy-, Ring A is an optionally substituted aryl or an optionally substituted heteroaryl containing at least one N, O, or S atom, and T is an optionally substituted amine chosen from:

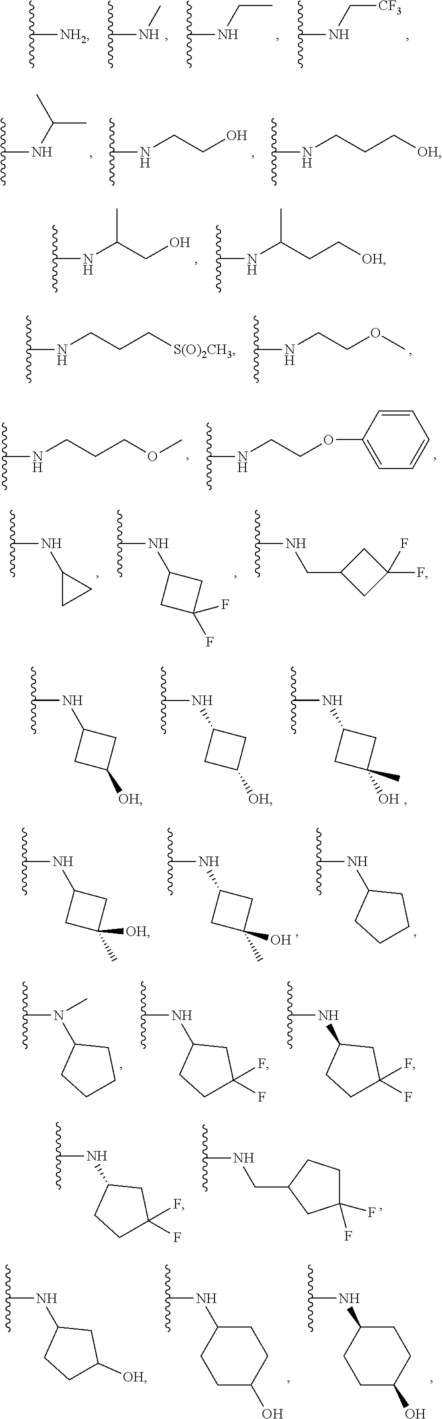

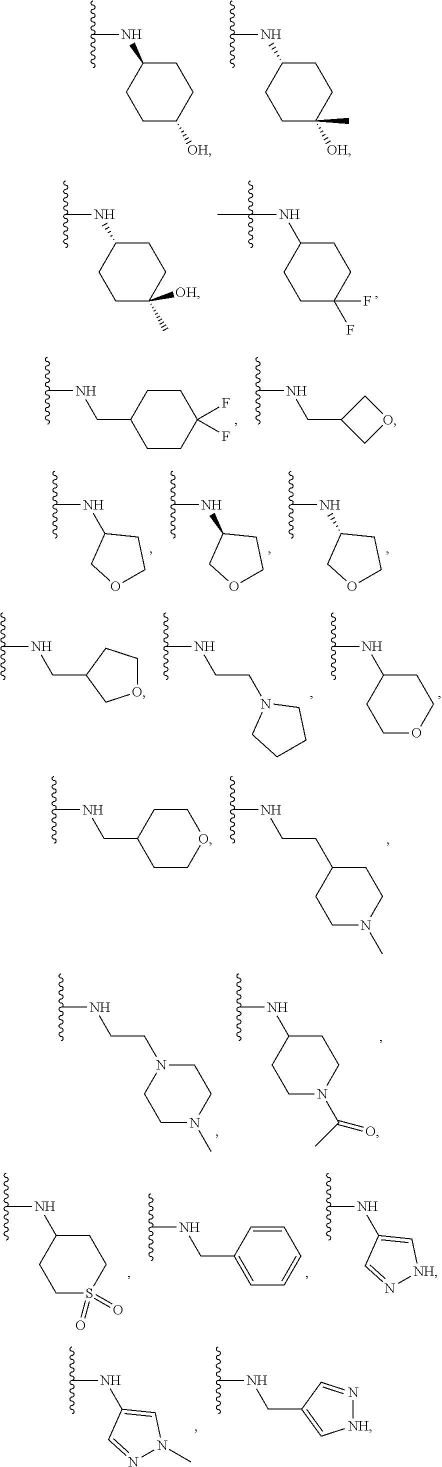

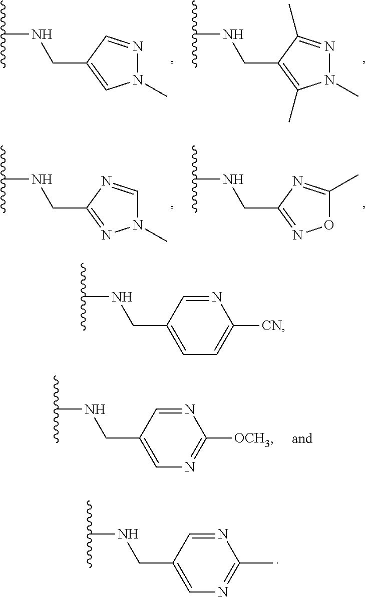

In some embodiments, U^{a} is -alkoxy-, Ring A is an optionally substituted aryl or an optionally substituted heteroaryl containing at least one N, O, or S atom, and T is an optionally substituted amine chosen from

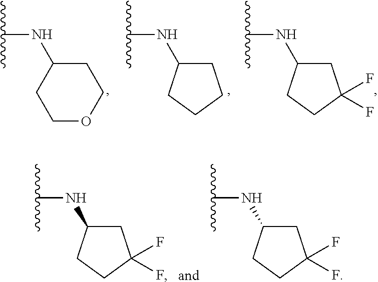

In some embodiments, Z² is chosen from —H, -alkyl, -alkenyl, —C(O)NHR¹, —C(O)NR¹(alkyl), and —C(O)O(alkyl), wherein each alkyl and alkenyl group is optionally substituted. In some embodiments, Z² is chosen from —H, —C₁₋₆ alkyl, —C₂₋₆ alkenyl, —C(O)NHR¹, —C(O)NR¹(C₁₋₆ alkyl), and —C(O)O(C₁₋₆ alkyl), wherein each alkyl and alkenyl group is optionally substituted. In some embodiments, Z² is chosen from —H, —C₁₋₆ alkyl, and —C₂₋₆ alkenyl, wherein each alkyl and alkenyl group is optionally substituted.

In some embodiments, each optional substituent of Z² is independently chosen from: (i) —C₁₋₆ alkoxy wherein the alkyl is optionally substituted with 1, 2, or 3 —OH or —C(O)NH₂, (ii) —OH, (iii) —C(O)—O—C₁₋₆ alkyl, and (iv) —C(O)—C₁₋₆ alkyl.

In some embodiments, Z² is -alkyl optionally substituted with 1 or 2 groups independently chosen from —OCH₂CH₂OH, —OH, —OCH₂C(O)NH₂ and —C(O)OCH₂CH₃, or Z² is -alkenyl substituted with —C(O)CH₂CH₃. In some embodiments, Z² is —C₁₋₆ alkyl optionally substituted with 1 or 2 groups independently chosen from —OCH₂CH₂OH, —OH, —OCH₂C(O)NH₂ and —C(O)OCH₂CH₃, or Z² is —C₂₋₆ alkenyl substituted with —C(O)CH₂CH₃. In some embodiments, Z² is chosen from —C₁₋₆ alkyl and —C₂₋₆ alkenyl. In some embodiments, Z² is —CH₃. In some embodiments, Z² is —CH═CH₂.

In some embodiments, V is chosen from —H, -halo, —OH, -alkyl, and -alkoxy. In some embodiments, V is chosen from —H, -halo, —OH, —C₁₋₆ alkyl, and —C₁₋₆ alkoxy. In some embodiments, V is chosen from —H and -halo. In some embodiments, V is —H. In some embodiments, V is -halo. In some embodiments, V is chosen from —F, —Cl, and Br. In some embodiments, V is —F. In some embodiments, V is —OH. In some embodiments, V is —C₁₋₆ alkyl, such as —CH₃. In some embodiments, V is —C₁₋₆ alkoxy, such as —OCH₃.

In some embodiments, U^{a} is -alkoxy-, Ring A is an optionally substituted aryl or an optionally substituted heteroaryl containing at least one N, O, or S atom, and T is an optionally substituted amine. In some embodiments, U^{a} is -alkoxy-, Ring A is an optionally substituted aryl or an optionally substituted heteroaryl containing at least one N, O, or S atom, V is —H, and T is an optionally substituted amine chosen from:

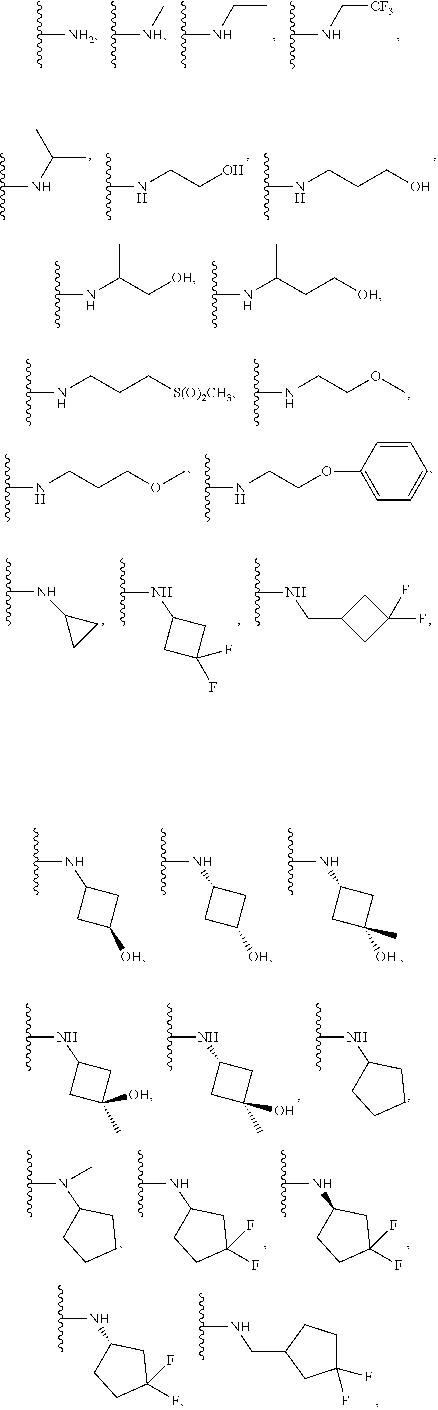

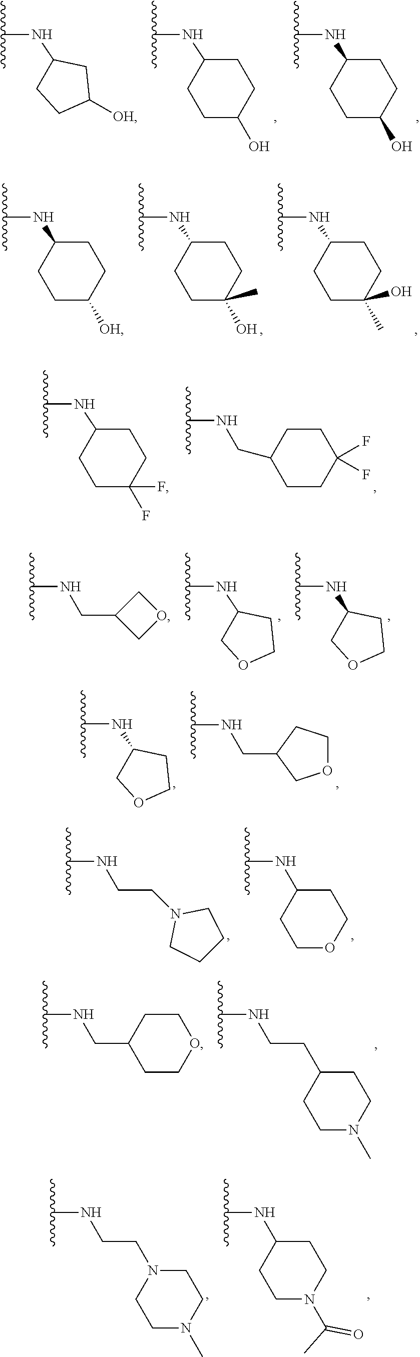

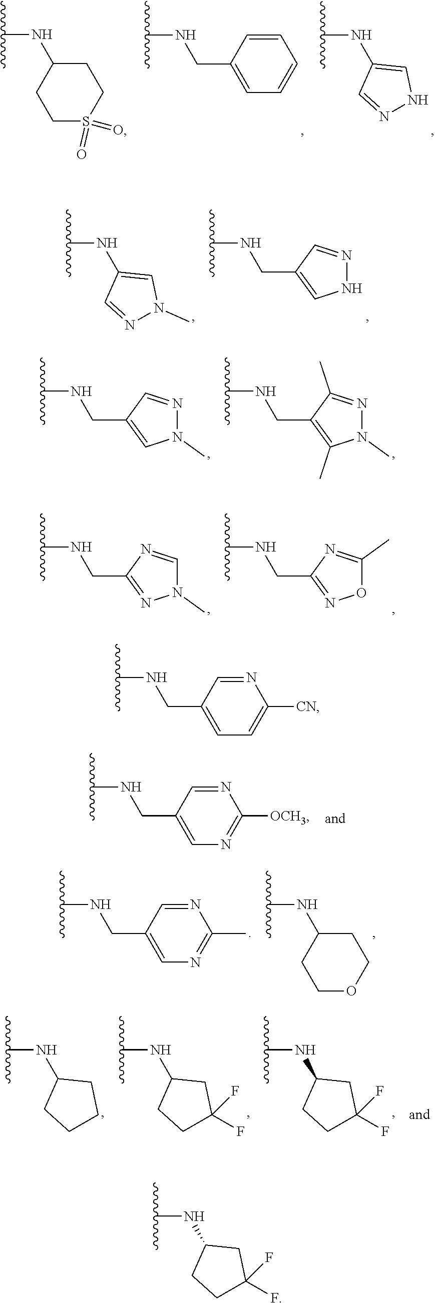

In some embodiments, U^{a} is -alkoxy-, Ring A is an optionally substituted aryl or an optionally substituted heteroaryl containing at least one N, O, or S atom, V is —H, and T is an optionally substituted amine chosen from

In some embodiments, M is —O— or —C(R²)₂—. In some embodiments, M is —O— or —CH₂—. In some embodiments, M is —O—.

In some embodiments, Y¹ and Y² are each independently chosen from —H, —OH, and -halo. In some embodiments, Y¹ and Y² are each independently chosen from —H, —OH, —F, —Cl, and —Br. In some embodiments, Y¹ and Y² are each independently chosen from —H, —OH, and —F.

In some embodiments, Y³ is —OH or —H. In some embodiments, Y³ is —OH. In some embodiments, Y³ is —H.

In some embodiments, Y⁴ is chosen from —H, -halo, —OH, and alkyl optionally substituted with 1, 2, or 3 groups independently chosen from -halo and —OH. In some embodiments, Y⁴ is chosen from —H, —F, —Cl, —Br, —OH, and C₁₋₆ alkyl optionally substituted with 1, 2, or 3 groups independently chosen from —F, —Cl, —Br, and —OH. In some embodiments, Y⁴ is chosen from —H, —F, —Cl, and C₁₋₆ alkyl optionally substituted with 1, 2, or 3 groups independently chosen from —F and —Cl. In some embodiments, Y⁴ is chosen from —H and —F. In some embodiments, Y⁴ is —H.

In some embodiments, Y¹ is —H and Y² is —OH. In some embodiments, Y¹ is —OH and Y² is —H. In some embodiments, Y¹ is —F and Y² is —OH. In some embodiments, Y¹ is —F and Y² is —H. In some embodiments, both Y¹ and Y² are —H. In some embodiments, both Y¹ and Y² are —F.

In some embodiments, Y³ is —H and Y⁴ is —OH. In some embodiments, Y³ is —OH and Y⁴ is —H. In some embodiments, Y³ is —F and Y⁴ is —OH. In some embodiments, Y³ is —OH and Y⁴ is —F.

In some embodiments, Y¹ and Y³ are both —OH. In some embodiments, Y¹ is —OH and Y³ is —H. In some embodiments, Y¹ is —H and Y³ is —OH.

In some embodiments, Y² and Y⁴ are both —H. In some embodiments, Y² is —OH and Y⁴ is —H. In some embodiments, Y² is —OH and Y⁴ is —H.

In some embodiments, Z¹ is chosen from —H, -halo, —OH, and -alkyl optionally substituted with 1, 2, or 3 groups independently chosen from —OH and -halo. In some embodiments, Z¹ is chosen from —H, -halo, —OH, and —C₁₋₆ alkyl optionally substituted with 1, 2, or 3 groups independently chosen from —OH and -halo. In some embodiments, Z¹ is chosen from —H, -halo, —OH, and —C₁₋₆ alkyl. In some embodiments, Z¹ is chosen from —H, —F, and —CH₃. In some embodiments, Z¹ is —H. In some embodiments, Z¹ is —F. In some embodiments, Z¹ is -CH₃.

In some embodiments, M is —O—, Y¹ and Y³ are both —H, and Y² and Y⁴ are both —OH. In some embodiments, M is —O—, Y¹ and Y³ are both —H, Y² and Y⁴ are both —OH, Z¹ is H, and Z² is an alkyl, such as a —CH₃.

In some embodiments, each R¹ is independently chosen from —H and -alkyl. In some embodiments, each R¹ is independently chosen from —H and —C₁₋₆ alkyl. In some embodiments, each R¹ is —C₁₋₆ alkyl. In some embodiments, each R¹ is —H. In some embodiments, each R¹ is —CH₃.

In some embodiments, each R² is independently chosen from —H, -alkyl, and -cycloalkyl. In some embodiments, each R² is independently chosen from —H, —C₁₋₆ alkyl, and —C₃₋₁₀ cycloalkyl. In some embodiments, each R² is independently chosen from —H, —C₁₋₆ alkyl, and —C₃₋₆ cycloalkyl. In some embodiments, each R² is independently chosen from —H and —C₁₋₆ alkyl. In some embodiments, each R² is —H. In some embodiments, each R² is —CH₃. In some embodiments, one R² is —H and one R² is —CH₃.

In one aspect, provided herein is a compound of Formula (Ib):

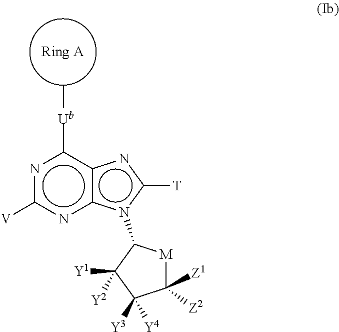

or a pharmaceutically acceptable salt, hydrate, or solvate thereof, wherein:
Ring A is an optionally substituted aryl;
U^{b} is: (i) -alkyl- or (ii) -alkenyl-;
T is chosen from —H, —OH, —O(alkyl)(aryl), optionally substituted heteroaryl that contains at least one N atom, —C(O)NR¹(cycloalkyl), and optionally substituted amine;
V is chosen from —H, -halo, —OH, -alkyl, and -alkoxy;
M is —O— or —C(R²)₂—;
Y¹ and Y² are each independently chosen from —H, —OH and -halo;
wherein at least one of Y¹, Y² or Y³ must be —OH;
Y³ is —OH or —H;
Y⁴ is chosen from —H, -halo, —OH, and -alkyl optionally substituted with 1, 2, or 3 groups independently chosen from -halo and —OH;
Z¹ is chosen from —H, -halo, —OH, and -alkyl optionally substituted with 1, 2, or 3 groups independently chosen from —OH and -halo;
Z² is -alkyl;
R¹ is chosen from —H and -alkyl; and
each R² is independently chosen from —H, -alkyl, and -cycloalkyl.

In some embodiments, Ring A is an optionally substituted aryl. In some embodiments, Ring A is an optionally substituted C₆₋₁₂ aryl. In some embodiments, Ring A is an optionally substituted C₆ aryl. In some embodiments, Ring A is C₆ aryl optionally substituted with 1, 2, or 3 groups independently chosen from: (i) —NO₂, (ii) —C₁₋₆ alkyl optionally substituted with 1, 2, or 3 —F atoms, (iii) —C₁₋₆ alkoxy optionally substituted with 1, 2, or 3 —F atoms, (iv) —CN, (v) -halo, and (vi) —C₃₋₁₀ cycloalkyl. In some embodiments, Ring A is C₆ aryl optionally substituted with 1, 2, or 3 groups independently chosen from —NO₂, —CH₃, —OCHF₂, —OCF₃, —OCH₂CF₃, —CN, —F, —Cl, —CHF₂, —CF₃, and -cyclopropyl. In some embodiments, Ring A is C₆ aryl optionally substituted with —NO₂.

In some embodiments, U^{b} is: (i) -alkyl- or (ii) -alkenyl-. In some embodiments, U^{b} is: (i) —C₁₋₆ alkyl- or (ii) —C₂₋₆ alkenyl-. In some embodiments, U^{b} is —CH₂—, —CH₂CH₂— or —CH═CH—.

In some embodiments, Z² is -alkyl. In some embodiments, Z² is —C₁₋₆ alkyl. In some embodiments, Z² is chosen from —CH₃ and —CH₂CH₃. In some embodiments, Z² is —CH₃.

In some embodiments, T is chosen from —H, —OH, —O(alkyl)(aryl), optionally substituted heteroaryl that contains at least one N atom, —C(O)NR¹(cycloalkyl), and optionally substituted amine. In some embodiments, T is chosen from —H, —OH, —O(C₁₋₆ alkyl)(C₆₋₁₂ aryl), optionally substituted 5-12 membered heteroaryl that contains at least one N atom, —C(O)NR¹(C₃₋₁₀ cycloalkyl), and optionally substituted amine.

In some embodiments, T is chosen from —H, —OH, —O(C₁₋₆ alkyl)(C₆₋₁₂ aryl), optionally substituted 5-12 membered heteroaryl that contains at least one N atom, —C(O)NR¹(C₃₋₁₀ cycloalkyl), and optionally substituted amine. In some embodiments, T is chosen from —H, —O(CH₂)(C₆ aryl), —C(O)NR¹(C₃₋₅ cycloalkyl), optionally substituted 5 membered heteroaryl that contains at least one N atom, and an optionally substituted amine. In some embodiments, T is chosen from —H, —O(CH₂)(C₆ aryl), —C(O)NR¹(C₅ cycloalkyl), 5 membered heteroaryl that contains at least one N atom, and an optionally substituted amine. In some embodiments, T is chosen from —H and an optionally substituted amine.

In some embodiments, T is —H. In some embodiments, T is an optionally substituted amine chosen from —NH₂, —NR¹(C₁₋₄ alkyl), —NR¹(C₃₋₆ cycloalkyl), —NR¹CH₂(C₃₋₆ cycloalkyl), —NR₁(5-6 membered heterocyclyl), —NR¹CH₂(5-6 membered heterocyclyl), —NR¹(5-6 membered heteroaryl), —NR¹CH₂(5-6 membered heteroaryl), —NR¹(C₆ aryl), and —NR¹CH₂(C₆ aryl),
wherein each alkyl, cycloalkyl, heterocyclyl, heteroaryl, or aryl is optionally substituted with 1, 2, or 3 groups independently chosen from —OH, -halo, —CN, —CH₃, —C(O)CH₃, —OCH₃, —SO₂CH₃, and —O-phenyl.

In some embodiments, V is chosen from —H, -halo, —OH, -alkyl, and -alkoxy. In some embodiments, V is chosen from —H, -halo, —OH, —C₁₋₆ alkyl, and —C₁₋₆ alkoxy. In some embodiments, V is chosen from —H and -halo. In some embodiments, V is —H. In some embodiments, V is -halo. In some embodiments, V is chosen from —F, —Cl, and Br. In some embodiments, V is —F. In some embodiments, V is —OH. In some embodiments, V is —C₁₋₆ alkyl, such as —CH₃. In some embodiments, V is —C₁₋₆ alkoxy, such as —OCH₃.

In some embodiments, M is —O— or —C(R²)₂—. In some embodiments, M is —O— or —CH₂—. In some embodiments, M is —O—.

In some embodiments, Y¹ and Y² are each independently chosen from —H, —OH, and -halo. In some embodiments, Y¹ and Y² are each independently chosen from —H, —OH, —F, —Cl, and —Br. In some embodiments, Y¹ and Y² are each independently chosen from —H, —OH, and —F.

In some embodiments, Y³ is —OH or —H. In some embodiments, Y³ is —OH. In some embodiments, Y³ is —H.

In some embodiments, Y⁴ is chosen from —H, -halo, —OH, and alkyl optionally substituted with 1, 2, or 3 groups independently chosen from -halo and —OH. In some embodiments, Y⁴ is chosen from —H, —F, —Cl, —Br, —OH, and C₁₋₆ alkyl optionally substituted with 1, 2, or 3 groups independently chosen from —F, —Cl, —Br, and —OH. In some embodiments, Y⁴ is chosen from —H, —F, —Cl, and C₁₋₆ alkyl optionally substituted with 1, 2, or 3 groups independently chosen from —F and —Cl. In some embodiments, Y⁴ is chosen from —H and —F. In some embodiments, Y⁴ is —H.

In some embodiments, Y¹ is —H and Y² is —OH. In some embodiments, Y¹ is —OH and Y² is —H. In some embodiments, Y¹ is —F and Y² is —OH. In some embodiments, Y¹ is —F and Y² is —H. In some embodiments, both Y¹ and Y² are —H. In some embodiments, both Y¹ and Y² are —F.

In some embodiments, Y³ is —H and Y⁴ is —OH. In some embodiments, Y³ is —OH and Y⁴ is —H. In some embodiments, Y³ is —F and Y⁴ is —OH. In some embodiments, Y³ is —OH and Y⁴ is —F.

In some embodiments, Y¹ and Y³ are both —OH. In some embodiments, Y¹ is —OH and Y³ is —H. In some embodiments, Y¹ is —H and Y³ is —OH.

In some embodiments, Y² and Y⁴ are both —H. In some embodiments, Y² is —OH and Y⁴ is —H. In some embodiments, Y² is —OH and Y⁴ is —H.

In some embodiments, Z¹ is chosen from —H, -halo, —OH, and -alkyl optionally substituted with 1, 2, or 3 groups independently chosen from —OH and -halo. In some embodiments, Z¹ is chosen from —H, -halo, —OH, and —C₁₋₆ alkyl optionally substituted with 1, 2, or 3 groups independently chosen from —OH and -halo. In some embodiments, Z¹ is chosen from —H, -halo, —OH, and —C₁₋₆ alkyl. In some embodiments, Z¹ is chosen from —H, —F, and —CH₃. In some embodiments, Z¹ is —H. In some embodiments, Z¹ is —F. In some embodiments, Z¹ is —CH₃.

In some embodiments, each R¹ is independently chosen from —H and -alkyl. In some embodiments, each R¹ is independently chosen from —H and —C₁₋₆ alkyl. In some embodiments, each R¹ is —C₁₋₆ alkyl. In some embodiments, each R¹ is —H. In some embodiments, each R¹ is —CH₃.

In some embodiments, each R² is independently chosen from —H, -alkyl, and -cycloalkyl. In some embodiments, each R² is independently chosen from —H, —C₁₋₆ alkyl, and —C₃₋₁₀ cycloalkyl. In some embodiments, each R² is independently chosen from —H, —C₁₋₆ alkyl, and —C₃₋₆ cycloalkyl. In some embodiments, each R² is independently chosen from —H and —C₁₋₆ alkyl. In some embodiments, each R² is —H. In some embodiments, each R² is —CH₃. In some embodiments, one R² is —H and one R² is —CH₃.

In one aspect, provided herein is a compound of Formula (Ic):

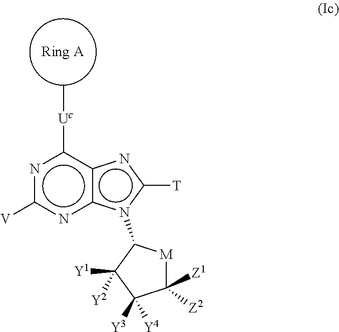

or a pharmaceutically acceptable salt, hydrate, or solvate thereof, wherein:
Ring A is optionally substituted aryl;
U^{c} is chosen from: (i) -alkyl-NR¹— wherein alkyl is attached to Ring A, (ii) —NR¹—, (iii) —CO—, (iv) —C(O)NR¹— wherein the N is attached to Ring A, and (v) —C(O)NR¹-alkyl- wherein the alkyl is attached to Ring A;
T is chosen from —H, —OH, —O(alkyl)(aryl), optionally substituted heteroaryl that contains at least one N atom, —C(O)NR¹(cycloalkyl), and optionally substituted amine;
V is chosen from —H, -halo, —OH, -alkyl, and -alkoxy;
M is —O— or —C(R²)₂—;
Y³ is —OH or —H;
Y¹ and Y² are each —H, —OH or -halo;
Y⁴ is chosen from —H, -halo, —OH, and -alkyl optionally substituted with 1, 2, or 3 groups independently chosen from -halo and —OH;
Z¹ is chosen from —H, -halo, —OH, and -alkyl optionally substituted with 1, 2, or 3 groups independently chosen from —OH and -halo;
Z₂ is -alkyl; and
each R¹ is independently chosen from —H and -alkyl; and
each R² is independently chosen from —H, -alkyl, and -cycloalkyl;
   with the proviso that the compound is not

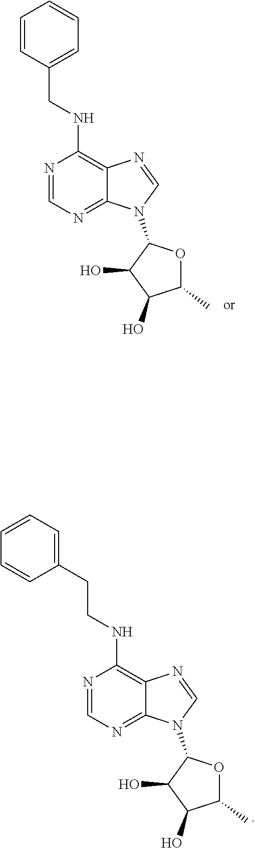 [SMILES: C[C@H]1O[C@@H](n2cnc3c(NCc4ccccc4)ncnc32)[C@H](O)[C@@H]1O ; C[C@H]1O[C@@H](n2cnc3c(NCCc4ccccc4)ncnc32)[C@H](O)[C@@H]1O]

In some embodiments, Ring A is optionally substituted aryl. In some embodiments, Ring A is an optionally substituted C₆₋₁₂ aryl. In some embodiments, Ring A is an optionally substituted C₆ aryl. In some embodiments, Ring A is C₆ aryl optionally substituted with 1, 2, or 3 groups independently chosen from: (i) —NO₂, (ii) —C₁₋₆ alkyl optionally substituted with 1, 2, or 3 —F atoms, (iii) —C₁₋₆ alkoxy optionally substituted with 1, 2, or 3 —F atoms, (iv) —CN, (v) -halo, and (vi) —C₃₋₁₀ cycloalkyl. In some embodiments, Ring A is C₆ aryl optionally substituted with 1, 2 or 3 groups independently chosen from —NO₂, —CH₃, —OCHF₂, —OCF₃, —OCH₂CF₃, —CN, —F, —Cl, —CHF₂, —CF₃, and -cyclopropyl. In some embodiments, Ring A is C₆ aryl optionally substituted with —NO₂.

In some embodiments, U^{c} is chosen from: (i) -alkyl-NR¹— wherein alkyl is attached to Ring A, (ii) —NR¹—, (iii) —CO—, (iv) —C(O)NR¹— wherein the N is attached to Ring A, and (v) —C(O)NR¹-alkyl- wherein the alkyl is attached to Ring A. In some embodiments, U^{c} is chosen from: (i) —C₁₋₆ alkyl-NR¹— wherein alkyl is attached to Ring A, (ii) —NR¹—, (iii) —CO—, (iv) —C(O)NR¹— wherein the N is attached to Ring A, and (v) —C(O)NR¹—C₁₋₆ alkyl- wherein the alkyl is attached to Ring A. In some embodiments, U^{c} is chosen from: (i) —C₁₋₆ alkyl-NH— wherein alkyl is attached to Ring A, (ii) —NH—, (iii) —CO—, (iv) —C(O)NH— wherein the N is attached to Ring A, and (v) —C(O)NH—C₁₋₆ alkyl- wherein the alkyl is attached to Ring A.

In some embodiments, U^{c} is chosen from -alkyl-NR¹— wherein alkyl is attached to Ring A and —NR¹—. In some embodiments, U^{c} is chosen from -alkyl-NR¹— wherein alkyl is attached to Ring A, and —NR¹—. In some embodiments, U^{c} is chosen from —C₁₋₆ alkyl-NR¹— wherein alkyl is attached to Ring A and —NR¹—. In some embodiments, U^{c} is chosen from —(CH₂)₁₋₂—NR¹— wherein —(CH₂)₁₋₂— is attached to Ring A and —NH—. In some embodiments, U^{c} is —(CH₂)₁₋₂—NR¹— wherein —(CH₂)₁₋₂— is attached to Ring A.

In some embodiments, Z² is -alkyl. In some embodiments, Z² is —C₁₋₆ alkyl. In some embodiments, Z² is chosen from —CH₃ and —CH₂CH₃. In some embodiments, Z² is —CH₃.

In some embodiments, T is chosen from —H, —OH, —O(alkyl)(aryl), optionally substituted heteroaryl that contains at least one N atom, —C(O)NR¹(cycloalkyl), and optionally substituted amine. In some embodiments, T is chosen from —H, —OH, —O(C₁₋₆ alkyl)(C₆₋₁₂ aryl), optionally substituted 5-12 membered heteroaryl that contains at least one N atom, —C(O)NR¹(C₃₋₁₀ cycloalkyl), and optionally substituted amine. In some embodiments, T is chosen from —H, —O(CH₂)(C₆ aryl), —C(O)NR¹(C₃₋₅ cycloalkyl), optionally substituted 5 membered heteroaryl that contains at least one N atom, and an optionally substituted amine. In some embodiments, T is chosen from —H, —O(CH₂)(C₆ aryl), —C(O)NR¹(C₅ cycloalkyl), 5 membered heteroaryl that contains at least one N atom, and an optionally substituted amine. In some embodiments, T is chosen from —H and an optionally substituted amine.

In some embodiments, T is —H. In some embodiments, T is an optionally substituted amine chosen from —NH₂, —NR¹(C₁₋₄ alkyl), —NR¹(C₃₋₆ cycloalkyl), —NR¹CH₂(C₃₋₆ cycloalkyl), —NR₁(5-6 membered heterocyclyl), —NR¹CH₂(5-6 membered heterocyclyl), —NR¹(5-6 membered heteroaryl), —NR¹CH₂(5-6 membered heteroaryl), —NR¹(C₆ aryl), and —NR¹CH₂(C₆ aryl),
wherein each alkyl, cycloalkyl, heterocyclyl, heteroaryl, or aryl is optionally substituted with 1, 2, or 3 groups independently chosen from —OH, -halo, —CN, —CH₃, —C(O)CH₃, —OCH₃, —SO₂CH₃, and —O-phenyl.

In some embodiments, V is chosen from —H, -halo, —OH, -alkyl, and -alkoxy. In some embodiments, V is chosen from —H, -halo, —OH, —C₁₋₆ alkyl, and —C₁₋₆ alkoxy. In some embodiments, V is chosen from —H and -halo. In some embodiments, V is —H. In some embodiments, V is -halo. In some embodiments, V is chosen from —F, —Cl, and Br. In some embodiments, V is —F. In some embodiments, V is —OH. In some embodiments, V is —C₁₋₆ alkyl, such as —CH₃. In some embodiments, V is —C₁₋₆ alkoxy, such as —OCH₃.

In some embodiments, M is —O— or —C(R²)₂—. In some embodiments, M is —O— or —CH₂—. In some embodiments, M is —O—.

In some embodiments, Y¹ and Y² are each independently chosen from —H, —OH, and -halo. In some embodiments, Y¹ and Y² are each independently chosen from —H, —OH, —F, —Cl, and —Br. In some embodiments, Y¹ and Y² are each independently chosen from —H, —OH, and —F.

In some embodiments, Y³ is —OH or —H. In some embodiments, Y³ is —OH. In some embodiments, Y³ is —H.

In some embodiments, Y⁴ is chosen from —H, -halo, —OH, and alkyl optionally substituted with 1, 2, or 3 groups independently chosen from -halo and —OH. In some embodiments, Y⁴ is chosen from —H, —F, —Cl, —Br, —OH, and C₁₋₆ alkyl optionally substituted with 1, 2, or 3 groups independently chosen from —F, —Cl, —Br, and —OH. In some embodiments, Y⁴ is chosen from —H, —F, —Cl, and C₁₋₆ alkyl optionally substituted with 1, 2, or 3 groups independently chosen from —F and —Cl. In some embodiments, Y⁴ is chosen from —H and —F. In some embodiments, Y⁴ is —H.

In some embodiments, Y¹ is —H and Y² is —OH. In some embodiments, Y¹ is —OH and Y² is —H. In some embodiments, Y¹ is —F and Y² is —OH. In some embodiments, Y¹ is —F and Y² is —H. In some embodiments, both Y¹ and Y² are —H. In some embodiments, both Y¹ and Y² are —F.

In some embodiments, Y³ is —H and Y⁴ is —OH. In some embodiments, Y³ is —OH and Y⁴ is —H. In some embodiments, Y³ is —F and Y⁴ is —OH. In some embodiments, Y³ is —OH and Y⁴ is —F.

In some embodiments, Y¹ and Y³ are both —OH. In some embodiments, Y¹ is —OH and Y³ is —H. In some embodiments, Y¹ is —H and Y³ is —OH.

In some embodiments, Y² and Y⁴ are both —H. In some embodiments, Y² is —OH and Y⁴ is —H. In some embodiments, Y² is —OH and Y⁴ is —H.

In some embodiments, Z¹ is chosen from —H, -halo, —OH, and -alkyl optionally substituted with 1, 2, or 3 groups independently chosen from —OH and -halo. In some embodiments, Z¹ is chosen from —H, -halo, —OH, and —C₁₋₆ alkyl optionally substituted with 1, 2, or 3 groups independently chosen from —OH and -halo. In some embodiments, Z¹ is chosen from —H, -halo, —OH, and —C₁₋₆ alkyl. In some embodiments, Z¹ is chosen from —H, —F, and —CH₃. In some embodiments, Z¹ is —H. In some embodiments, Z¹ is —F. In some embodiments, Z¹ is —CH₃.

In some embodiments, each R¹ is independently chosen from —H and -alkyl. In some embodiments, each R¹ is independently chosen from —H and —C₁₋₆ alkyl. In some embodiments, each R¹ is —C₁₋₆ alkyl. In some embodiments, each R¹ is —H. In some embodiments, each R¹ is —CH₃.

In some embodiments, each R² is independently chosen from —H, -alkyl, and -cycloalkyl. In some embodiments, each R² is independently chosen from —H, —C₁₋₆ alkyl, and —C₃₋₁₀ cycloalkyl. In some embodiments, each R² is independently chosen from —H, —C₁₋₆ alkyl, and —C₃₋₆ cycloalkyl. In some embodiments, each R² is independently chosen from —H and —C₁₋₆ alkyl. In some embodiments, each R² is —H. In some embodiments, each R² is —CH₃. In some embodiments, one R² is —H and one R² is —CH₃.

In one aspect, provided herein is a compound of Formula (Id):

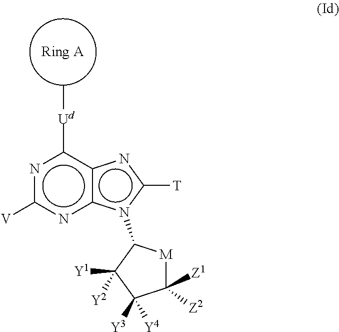

or a pharmaceutically acceptable salt, hydrate, or solvate thereof, wherein:
Ring A is C₆ aryl substituted with 1 or 2 groups independently chosen from —NO₂, —CN, —CH₂CN, -alkoxy optionally substituted with 1, 2, or 3-halo atoms, and 5 membered heteroaryl optionally substituted with 1 or 2 —CH₃ groups;
   U^{d} is chosen from: (i) -alkyl-S— wherein alkyl is attached to Ring A, (ii) —SO₂NR¹— wherein the N is attached to Ring A, and (iii) -alkyl-SO₂—NR¹— wherein the N is attached to Ring A;
   T is —H;
   V is chosen from —H, -halo, —OH, -alkyl, and -alkoxy;
   M is —O— or —C(R²)₂—;
   Y¹ and Y² are each independently chosen from —H, —OH and -halo;
   Y³ is —OH or —H;
   Y⁴ is chosen from —H, -halo, —OH, and -alkyl optionally substituted with 1, 2, or 3 groups independently chosen from -halo and —OH;
   Z¹ is chosen from —H, -halo, —OH, and -alkyl optionally substituted with 1, 2, or 3 groups independently chosen from —OH and -halo;
   Z² is chosen from —H, -alkyl, -alkenyl, —C(O)NHR¹, —C(O)NR¹(alkyl), and —C(O)O(alkyl), wherein each alkyl and alkenyl group is optionally substituted;
   each R¹ is independently chosen from —H and -alkyl; and
   each R² is independently chosen from —H, -alkyl, and -cycloalkyl;
      with the proviso that the compound is not

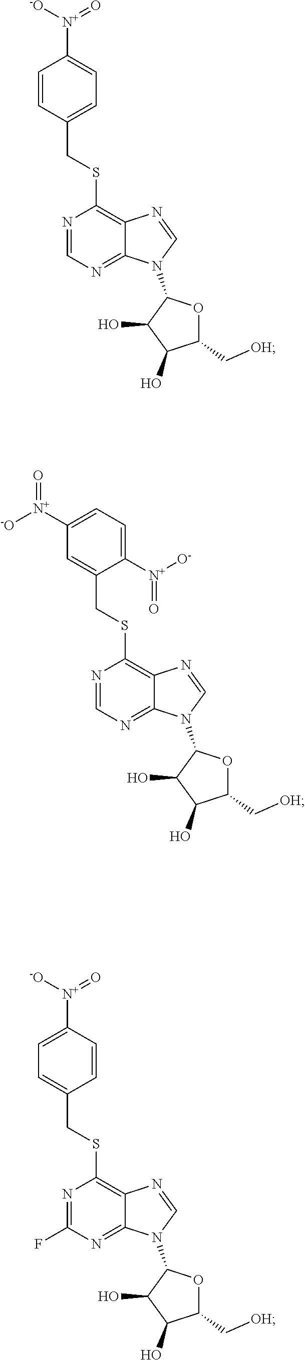

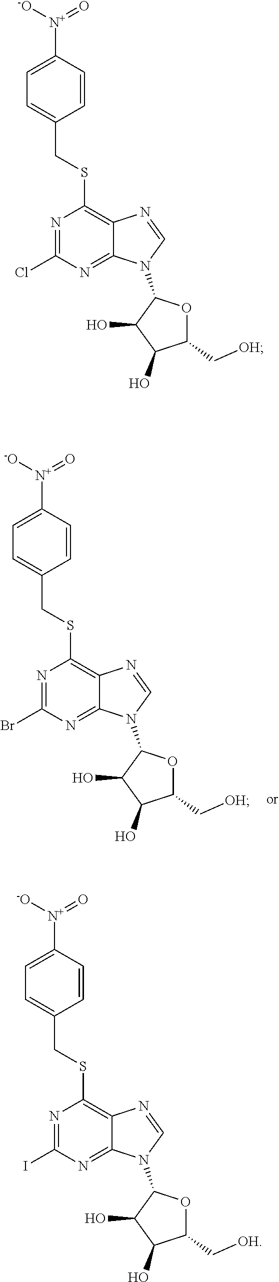

In some embodiments, Ring A is C₆ aryl substituted with 1 or 2 groups independently chosen from —NO₂, —CN, —CH₂CN, -alkoxy optionally substituted with 1, 2, or 3-halo atoms, and 5 membered heteroaryl optionally substituted with 1 or 2 —CH₃ groups. In some embodiments, Ring A is an optionally substituted C₆ aryl optionally substituted with 1 or 2 groups independently chosen from —NO₂, —CN, —CH₂CN, -alkoxy optionally substituted with 1, 2, or 3 —F atoms, and 5 membered heteroaryl optionally substituted with 1 —CH₃ group. In some embodiments, Ring A is an optionally substituted C₆ aryl optionally substituted with 1 or 2 groups independently chosen from —NO₂, —CN, —CH₂CN, —C₁₋₆ alkoxy optionally substituted with 1, 2, or 3 —F atoms, and 5 membered heteroaryl optionally substituted with 1 —CH₃ group. In some embodiments, Ring A is a C₆ aryl optionally substituted with 1 or 2 groups independently chosen from —NO₂, —CN, —CH₂CN, —OCHF₂, —OCH₂CF₃ and 1,2,4-oxadiazole optionally substituted with 1 —CH₃ group.

In some embodiments, U^{d} is chosen from: (i) -alkyl-S— wherein alkyl is attached to Ring A, (ii) —SO₂NR¹— wherein the N is attached to Ring A, and (iii) -alkyl-SO₂—NR¹— wherein the N is attached to Ring A.

In some embodiments, U^{d} is -alkyl-S— wherein alkyl is attached to Ring A. In some embodiments, U^{d} is —C₁₋₆ alkyl-S— wherein alkyl is attached to Ring A. In some embodiments, U^{d} is —(CH₂)₁₋₂—S— wherein —(CH₂)₁₋₂— is attached to Ring A.

In some embodiments, U^{d} is chosen from —SO₂NR¹— wherein the N is attached to Ring A, and —C₁₋₆ alkyl-SO₂—NR¹— wherein the N is attached to Ring A. In some embodiments, U^{d} is chosen from —SO₂NH— wherein the N is attached to Ring A, and —C₁₋₆ alkyl-SO₂—NH— wherein the N is attached to Ring A.

In some embodiments, Z² is chosen from —H, -alkyl, -alkenyl, —C(O)NHR¹, —C(O)NR¹(alkyl), and —C(O)O(alkyl), wherein each alkyl and alkenyl group is optionally substituted. In some embodiments, Z² is chosen from —H, —C₁₋₆ alkyl, —C₂₋₆ alkenyl, —C(O)NHR¹, —C(O)NR¹(C₁₋₆ alkyl), and —C(O)O(C₁₋₆ alkyl), wherein each alkyl and alkenyl group is optionally substituted.

In some embodiments, Z² is chosen from —H, -alkyl, and —C(O)O(alkyl), wherein each alkyl is optionally substituted. In some embodiments, Z² is chosen from optionally substituted -alkyl and —C(O)OCH₃. In some embodiments, Z² is chosen from optionally substituted —C₁₋₆ alkyl and —C(O)OCH₃.

In some embodiments, the optional substituents on the alkyl or alkenyl group of Z² are chosen from —OH, -halo, and —NHC(O)(C₁₋₆ alkyl). In some embodiments, the optional substituents on the alkyl or alkenyl group of Z² are chosen from —OH, -halo, and —NHC(O)(C₁₋₆ alkyl). In some embodiments, the alkyl or alkenyl groups of Z² are not substituted with a phosphonate group or a protected alcohol group.

In some embodiments, Z² is an optionally substituted -alkyl, wherein the alkyl is not substituted with a phosphonate group or a protected alcohol group. In some embodiments, Z² is an optionally substituted —C₁₋₆ alkyl, wherein the alkyl is not substituted with a phosphonate group or a protected alcohol group. In some embodiments, Z² is chosen from —CH₃, —CH₂OH, —CH₂F, and —CH₂NHC(O)CH₃.

In some embodiments, V is chosen from —H, -halo, —OH, -alkyl, and -alkoxy. In some embodiments, V is chosen from —H, -halo, —OH, —C₁₋₆ alkyl, and —C₁₋₆ alkoxy. In some embodiments, V is chosen from —H and -halo. In some embodiments, V is —H. In some embodiments, V is -halo. In some embodiments, V is chosen from —F, —Cl, and Br. In some embodiments, V is —F. In some embodiments, V is —OH. In some embodiments, V is —C₁₋₆ alkyl, such as —CH₃. In some embodiments, V is —C₁₋₆ alkoxy, such as —OCH₃.

In some embodiments, M is —O— or —C(R²)₂—. In some embodiments, M is —O— or —CH₂—. In some embodiments, M is —O—.

In some embodiments, Y¹ and Y² are each independently chosen from —H, —OH, and -halo. In some embodiments, Y¹ and Y² are each independently chosen from —H, —OH, —F, —Cl, and —Br. In some embodiments, Y¹ and Y² are each independently chosen from —H, —OH, and —F.

In some embodiments, Y³ is —OH or —H. In some embodiments, Y³ is —OH. In some embodiments, Y³ is —H.

In some embodiments, Y⁴ is chosen from —H, -halo, —OH, and alkyl optionally substituted with 1, 2, or 3 groups independently chosen from -halo and —OH. In some embodiments, Y⁴ is chosen from —H, —F, —Cl, —Br, —OH, and C₁₋₆ alkyl optionally substituted with 1, 2, or 3 groups independently chosen from —F, —Cl, —Br, and —OH. In some embodiments, Y⁴ is chosen from —H, —F, —Cl, and C₁₋₆ alkyl optionally substituted with 1, 2, or 3 groups independently chosen from —F and —Cl. In some embodiments, Y⁴ is chosen from —H and —F. In some embodiments, Y⁴ is —H.

In some embodiments, Y¹ is —H and Y² is —OH. In some embodiments, Y¹ is —OH and Y² is —H. In some embodiments, Y¹ is —F and Y² is —OH. In some embodiments, Y¹ is —F and Y² is —H. In some embodiments, both Y¹ and Y² are —H. In some embodiments, both Y¹ and Y² are —F.

In some embodiments, Y³ is —H and Y⁴ is —OH. In some embodiments, Y³ is —OH and Y⁴ is —H. In some embodiments, Y³ is —F and Y⁴ is —OH. In some embodiments, Y³ is —OH and Y⁴ is —F.

In some embodiments, Y¹ and Y³ are both —OH. In some embodiments, Y¹ is —OH and Y³ is —H. In some embodiments, Y¹ is —H and Y³ is —OH.

In some embodiments, Y² and Y⁴ are both —H. In some embodiments, Y² is —OH and Y⁴ is —H. In some embodiments, Y² is —OH and Y⁴ is —H.

In some embodiments, Z¹ is chosen from —H, -halo, —OH, and -alkyl optionally substituted with 1, 2, or 3 groups independently chosen from —OH and -halo. In some embodiments, Z¹ is chosen from —H, -halo, —OH, and —C₁₋₆ alkyl optionally substituted with 1, 2, or 3 groups independently chosen from —OH and -halo. In some embodiments, Z¹ is chosen from —H, -halo, —OH, and —C₁₋₆ alkyl. In some embodiments, Z¹ is chosen from —H, —F, and —CH₃. In some embodiments, Z¹ is —H. In some embodiments, Z¹ is —F. In some embodiments, Z¹ is —CH₃.

In some embodiments, each R¹ is independently chosen from —H and -alkyl. In some embodiments, each R¹ is independently chosen from —H and —C₁₋₆ alkyl. In some embodiments, each R¹ is —C₁₋₆ alkyl. In some embodiments, each R¹ is —H. In some embodiments, each R¹ is —CH₃.

In some embodiments, each R² is independently chosen from —H, -alkyl, and -cycloalkyl. In some embodiments, each R² is independently chosen from —H, —C₁₋₆ alkyl, and —C₃₋₁₀ cycloalkyl. In some embodiments, each R² is independently chosen from —H, —C₁₋₆ alkyl, and —C₃₋₆ cycloalkyl. In some embodiments, each R² is independently chosen from —H and —C₁₋₆ alkyl. In some embodiments, each R² is —H. In some embodiments, each R² is —CH₃. In some embodiments, one R² is —H and one R² is —CH₃.

In one aspect, provided herein is a compound of Formula (Ie):

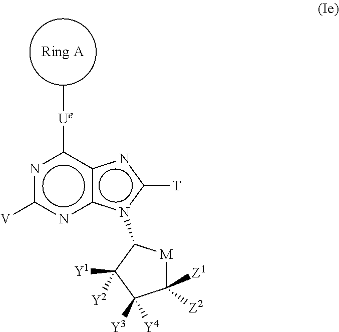

or a pharmaceutically acceptable salt, hydrate, or solvate thereof, wherein:
Ring A is chosen from optionally substituted aryl, optionally substituted heteroaryl containing at least one N, O, or S atom, optionally substituted heterocyclyl containing at least one N or O, and optionally substituted cycloalkyl;
U^{e} is a direct bond;
T is chosen from —H, —OH, —O(alkyl)(aryl), optionally substituted heteroaryl that contains at least one N atom, —C(O)NR¹(cycloalkyl), and optionally substituted amine;
V is chosen from —H, -halo, —OH, -alkyl, and -alkoxy;
M is —O— or —C(R²)₂—;
Y¹ and Y² are each independently chosen from —H, —OH, and -halo;
Y³ is —OH or —H;
Y⁴ is chosen from —H, -halo, —OH, and -alkyl optionally substituted with 1, 2, or 3 groups independently chosen from -halo and —OH;
Z¹ is chosen from —H, -halo, —OH, and -alkyl optionally substituted with 1, 2, or 3 groups independently chosen from —OH and -halo;
Z² is -alkyl;
R¹ is —H or -alkyl; and
each R² is independently chosen from —H, -alkyl, and -cycloalkyl.

In some embodiments, Ring A is chosen from optionally substituted aryl, optionally substituted heteroaryl containing at least one N, O, or S atom, optionally substituted heterocyclyl containing at least one N or O, and optionally substituted cycloalkyl. In some embodiments, Ring A is chosen from optionally substituted C₆₋₁₂ aryl, optionally substituted 5-12 membered heteroaryl containing at least one N, O, or S atom, optionally substituted 3-11 membered heterocyclyl containing at least one N or O, and optionally substituted C₃₋₁₀ cycloalkyl.

In some embodiments, Ring A is chosen from an optionally substituted C₆-10 cycloalkyl, an optionally substituted 4-10 membered heteroaryl containing at least one N, O, or S atom, and an optionally substituted 4-6 membered heterocyclyl containing at least one N or O. In some embodiments, Ring A is chosen from a C₁₀ cycloalkyl, a 10 membered heteroaryl, and a 4-5 membered heterocyclyl, each of which is optionally substituted.

In some embodiments, Ring A is chosen from

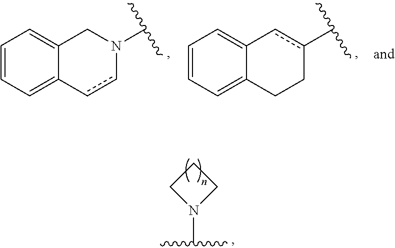

each of which is optionally substituted, wherein the [symbol] is a single or double bond, and wherein n is 1, 2 or 3.

In some embodiments, Ring A is

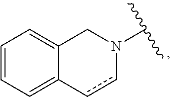

which is optionally substituted. In some embodiments, the [symbol] is a single bond.

In some embodiments, Ring A is

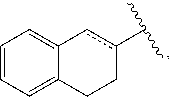

which is optionally substituted. In some embodiments, the [symbol] is a double bond.

In some embodiments, Ring A is

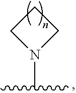

which is optionally substituted. In some embodiments, n is 1.

In some embodiments, Ring A is unsubstituted. In some embodiments, Ring A is optionally substituted with 1, 2, or 3 groups independently chosen from —NO₂, —CH₃, —OCHF₂, —OCF₃, —OCH₂CF₃, —CN, —F, —Cl, —CHF₂, —CF₃, and -aryl optionally substituted with —NO₂. In some embodiments, Ring A is substituted with —NO₂, —CN, and -aryl optionally substituted with —NO₂.

In some embodiments, Z² is -alkyl. In some embodiments, Z² is —C₁₋₆ alkyl. In some embodiments, Z² is chosen from —CH₃ and —CH₂CH₃. In some embodiments, Z² is —CH₃.

In some embodiments, T is chosen from —H, —OH, —O(alkyl)(aryl), optionally substituted heteroaryl that contains at least one N atom, —C(O)NR¹(cycloalkyl), and optionally substituted amine. In some embodiments, T is chosen from —H, —OH, —O(C₁₋₆ alkyl)(C₆₋₁₀ aryl), optionally substituted 5-12 membered heteroaryl that contains at least one N atom, —C(O)NR¹(C₃₋₁₀ cycloalkyl), and optionally substituted amine. In some embodiments, T is chosen from —H, —OH, —O(C₁₋₄ alkyl)(C₆ aryl), optionally substituted 5-6 membered heteroaryl that contains at least one N atom, —C(O)NR¹(C₃₋₆ cycloalkyl), and optionally substituted amine.

In some embodiments, T is chosen from —H and optionally substituted amine. In some embodiments, T is —H.

In some embodiments, T is an optionally substituted amine. In some embodiments, T is an optionally substituted amine chosen from —NH₂, —NR¹(C₁₋₆ alkyl), —NR¹(C₃₋₁₀ cycloalkyl), —NR¹CH₂(C₃₋₁₀ cycloalkyl), —NR¹(3-11 membered heterocyclyl), —NR¹CH₂(3-11 membered heterocyclyl), —NR¹(3-12 membered heteroaryl), —NR¹CH₂(3-12 membered heteroaryl), —NR¹(C₆-12 aryl), and —NR¹CH₂(C₆₋₁₂ aryl), wherein each alkyl, cycloalkyl, heterocyclyl, heteroaryl, or aryl is optionally substituted.

In some embodiments, T is an optionally substituted amine chosen from —NH₂, —NR¹(C₁₋₄ alkyl), —NR¹(C₃₋₆ cycloalkyl), —NR¹CH₂(C₃₋₆ cycloalkyl), —NR¹(5-6 membered heterocyclyl), —NR¹CH₂(5-6 membered heterocyclyl), —NR¹(5-6 membered heteroaryl), —NR¹CH₂(5-6 membered heteroaryl), —NR¹(C₆ aryl), and —NR¹CH₂(C₆ aryl), wherein each alkyl, cycloalkyl, heterocyclyl, heteroaryl, or aryl is optionally substituted with 1, 2, or 3 groups independently chosen from —OH, -halo, —CN, —CH₃, —C(O)CH₃, —OCH₃, —SO₂CH₃, and —O-phenyl.

In some embodiments, T is an optionally substituted amine chosen from:
(i) —N(R¹)₂,
(ii) —NR¹(C₁₋₆ alkyl) wherein the alkyl is optionally substituted with 1, 2, or 3 groups independently chosen from -halo, —OH, —SO₂CH₃, —OCH₃, and —O(C₆ aryl),
(iii) —NR¹(C₃₋₆ cycloalkyl) wherein the cycloalkyl is optionally substituted with 1, 2, or 3 groups independently chosen from —CH₃, -halo, and —OH,
(iv) —NR¹CH₂(C₃₋₆ cycloalkyl) wherein the cycloalkyl is optionally substituted with 1, 2, or 3 groups independently chosen from —CH₃, -halo, and —OH,
(v) —NR¹(5-6 membered heterocyclyl) wherein the heterocyclyl is optionally substituted with 1, 2, or 3 groups independently chosen from —C(O)CH₃, —CH₃, -halo, and —OH,
(iv) —NR¹CH₂(5-6 membered heterocyclyl) wherein the heterocyclyl is optionally substituted with 1, 2, or 3 groups independently chosen from —C(O)CH₃, —CH₃, -halo, and —OH,
(v) —NR¹CH₂CH₂(5-6 membered heterocyclyl) wherein the heterocyclyl is optionally substituted with 1, 2, or 3 groups independently chosen from —C(O)CH₃, —CH₃, -halo, and —OH,
(vi) —NR¹(5-6 membered heteroaryl) wherein the heteroaryl is optionally substituted with 1, 2, or 3 groups independently chosen from —CH₃, —CN, and —OCH₃,
(vii) —NR¹(CH₂)₁₋₂(5-6 membered heteroaryl) wherein the heteroaryl is optionally substituted with 1, 2, or 3 groups independently chosen from —CH₃, —CN, and —OCH₃,
(viii) —NR¹(C₆ aryl) wherein the aryl is optionally substituted with 1, 2, or 3 groups independently chosen from —CH₃, —CN, and —OCH₃,
(ix) —NR¹(CH₂)₁₋₂(C₆ aryl) wherein the aryl is optionally substituted with 1, 2, or 3 groups independently chosen from —CH₃, —CN, and —OCH₃.

In some embodiments, T is an optionally substituted amine chosen from:

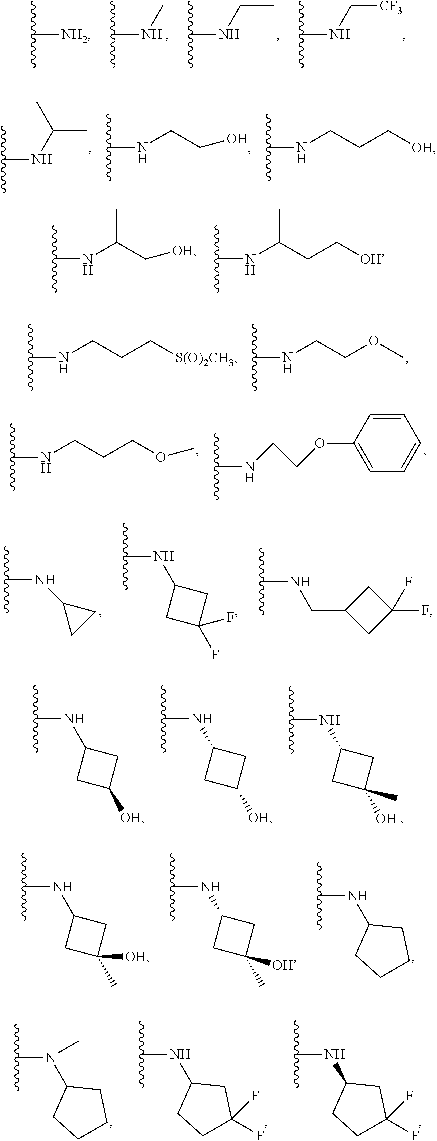

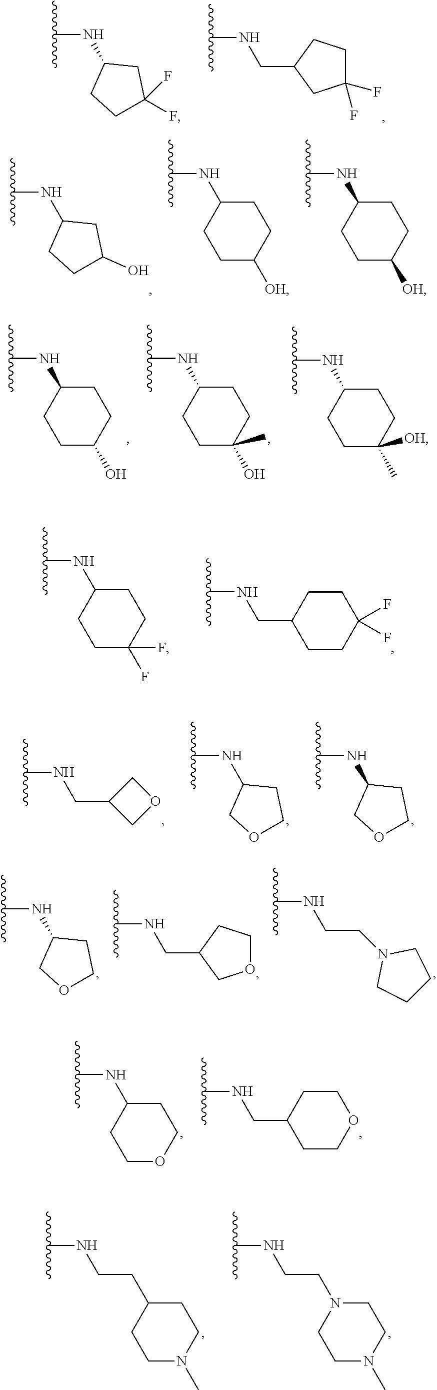

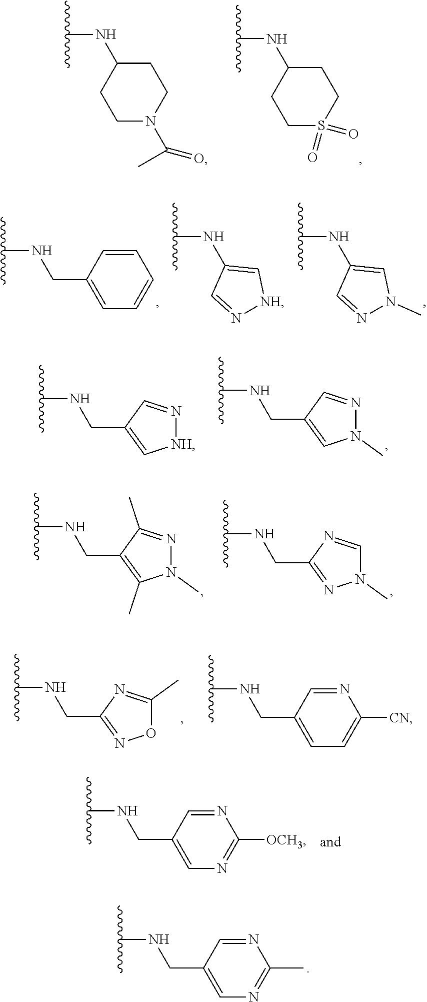

In some embodiments, T is an optionally substituted amine chosen from:

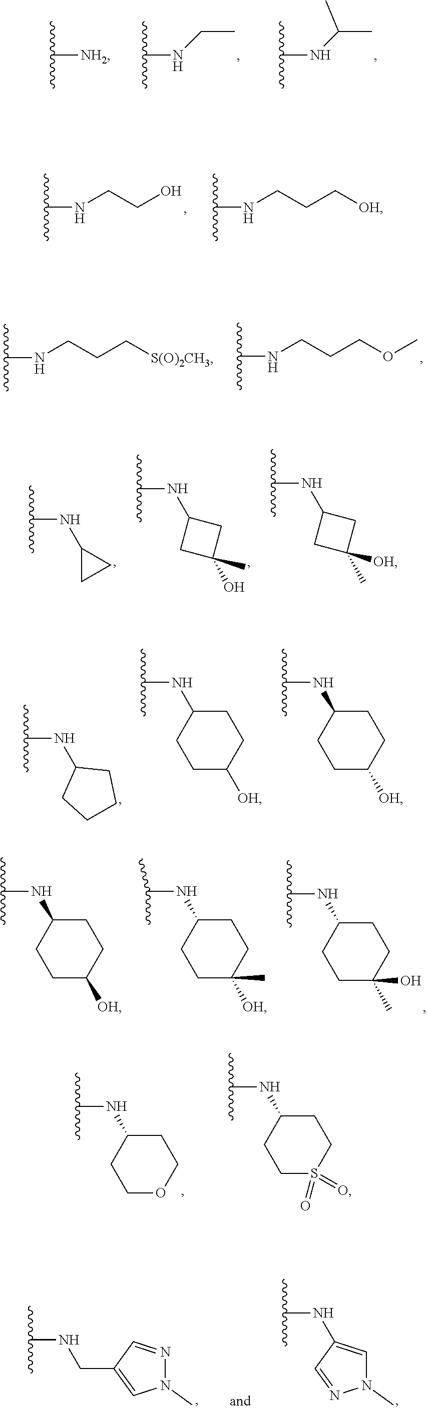

In some embodiments, V is chosen from —H, -halo, —OH, -alkyl, and -alkoxy. In some embodiments, V is chosen from —H, -halo, —OH, —C₁₋₆ alkyl, and —C₁₋₆ alkoxy. In some embodiments, V is chosen from —H and -halo. In some embodiments, V is —H. In some embodiments, V is -halo. In some embodiments, V is chosen from —F, —Cl, and Br. In some embodiments, V is —F. In some embodiments, V is —OH. In some embodiments, V is —C₁₋₆ alkyl, such as —CH₃. In some embodiments, V is —C₁₋₆ alkoxy, such as —OCH₃.

In some embodiments, M is —O— or —C(R²)₂—. In some embodiments, M is —O— or —CH₂—. In some embodiments, M is —O—.

In some embodiments, Y¹ and Y² are each independently chosen from —H, —OH, and -halo. In some embodiments, Y¹ and Y² are each independently chosen from —H, —OH, —F, —Cl, and —Br. In some embodiments, Y¹ and Y² are each independently chosen from —H, —OH, and —F.

In some embodiments, Y³ is —OH or —H. In some embodiments, Y³ is —OH. In some embodiments, Y³ is —H.

In some embodiments, Y⁴ is chosen from —H, -halo, —OH, and alkyl optionally substituted with 1, 2, or 3 groups independently chosen from -halo and —OH. In some embodiments, Y⁴ is chosen from —H, —F, —Cl, —Br, —OH, and C₁₋₆ alkyl optionally substituted with 1, 2, or 3 groups independently chosen from —F, —Cl, —Br, and —OH. In some embodiments, Y⁴ is chosen from —H, —F, —Cl, and C₁₋₆ alkyl optionally substituted with 1, 2, or 3 groups independently chosen from —F and —Cl. In some embodiments, Y⁴ is chosen from —H and —F. In some embodiments, Y⁴ is —H.

In some embodiments, Y¹ is —H and Y² is —OH. In some embodiments, Y¹ is —OH and Y² is —H. In some embodiments, Y¹ is —F and Y² is —OH. In some embodiments, Y¹ is —F and Y² is —H. In some embodiments, both Y¹ and Y² are —H. In some embodiments, both Y¹ and Y² are —F.

In some embodiments, Y³ is —H and Y⁴ is —OH. In some embodiments, Y³ is —OH and Y⁴ is —H. In some embodiments, Y³ is —F and Y⁴ is —OH. In some embodiments, Y³ is —OH and Y⁴ is —F.

In some embodiments, Y¹ and Y³ are both —OH. In some embodiments, Y¹ is —OH and Y³ is —H. In some embodiments, Y¹ is —H and Y³ is —OH.

In some embodiments, Y² and Y⁴ are both —H. In some embodiments, Y² is —OH and Y⁴ is —H. In some embodiments, Y² is —OH and Y⁴ is —H.

In some embodiments, Z¹ is chosen from —H, -halo, —OH, and -alkyl optionally substituted with 1, 2, or 3 groups independently chosen from —OH and -halo. In some embodiments, Z¹ is chosen from —H, -halo, —OH, and —C₁₋₆ alkyl optionally substituted with 1, 2, or 3 groups independently chosen from —OH and -halo. In some embodiments, Z¹ is chosen from —H, -halo, —OH, and —C₁₋₆ alkyl. In some embodiments, Z¹ is chosen from —H, —F, and —CH₃. In some embodiments, Z¹ is —H. In some embodiments, Z¹ is —F. In some embodiments, Z¹ is —CH₃.

In some embodiments, each R¹ is independently chosen from —H and -alkyl. In some embodiments, each R¹ is independently chosen from —H and —C₁₋₆ alkyl. In some embodiments, each R¹ is —C₁₋₆ alkyl. In some embodiments, each R¹ is —H. In some embodiments, each R¹ is —CH₃.

In some embodiments, each R² is independently chosen from —H, -alkyl, and -cycloalkyl. In some embodiments, each R² is independently chosen from —H, —C₁₋₆ alkyl, and —C₃₋₁₀ cycloalkyl. In some embodiments, each R² is independently chosen from —H, —C₁₋₆ alkyl, and —C₃₋₆ cycloalkyl. In some embodiments, each R² is independently chosen from —H and —C₁₋₆ alkyl. In some embodiments, each R² is —H. In some embodiments, each R² is —CH₃. In some embodiments, one R² is —H and one R² is —CH₃.

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to Ring A of Formula (I) may be combined with every description, variation, embodiment, or aspect of U (or U^{a}, U^{b}, U^{c}, U^{d}, U^{e}), T, V, M, Y¹, Y², Y³, Y⁴, Z¹ and Z², the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (Ia), (Ib), (Ic), (Id), and (Ie), and are equally described, the same as if each and every description, variation, embodiment, or aspect were separately and individually listed for all formulae.

In some embodiments, provided is a compound selected from the compounds in Table 1 or a pharmaceutically acceptable salt, hydrate, or solvate thereof.

Lengthy table referenced here
US20260184712A1-20260702-T00001
Please refer to the end of the specification for access instructions.

It is understood that in the present description, combinations of substituents and/or variables of the depicted formulae are permissible only if such contributions result in stable compounds.

In one embodiment, the present disclosure also relates to salts, hydrates, solvates, enantiomers, isomers (including optical, geometric and tautomeric isomers), polymorphs, multi-component complexes, liquid crystals, prodrugs of any of the compounds disclosed herein, for example compounds of Formula (I) and in Table 1, as well as isotopically-labeled compounds.

In one embodiment, the present disclosure relates to enantiomers and isomers (including optical, geometric and tautomeric isomers) of the compounds disclosed herein, for example compounds of Formula (I) and in Table 1. Indeed, the compounds disclosed herein may contain an asymmetric center and thus may exist as different stereoisomeric forms. Accordingly, the present disclosure includes all possible stereoisomers and includes not only racemic compounds but the individual enantiomers and their nonracemic mixtures as well. When a compound is desired as a single enantiomer, such may be obtained by stereospecific synthesis, by resolution of the final product or any convenient intermediate compound, or by chiral chromatographic methods as each are known in the art. Resolution of the final product, an intermediate compound, or a starting material may be performed by any suitable method known in the art. In one embodiment, the present disclosure relates to enantiomers and isomers (including optical, geometric and tautomeric isomers) of compounds of formula I and subformula thereof.

Furthermore, all compounds of the present disclosure that exist in free base or acid form can be converted to their pharmaceutically acceptable salts by treatment with the appropriate inorganic or organic base or acid by methods known to one skilled in the art. Salts of the compounds of the present disclosure can be converted to their free base or acid form by standard techniques.

In addition, the compounds and salts of the present disclosure may exist, as solvates. Solvates can be prepared from different solvents, including ethanol and/or water, by methods known to one skilled in the art. When the solvent is water, the term “hydrate” may be used. In some embodiments, the solvate or hydrate is a compound of the present disclosure such as a compound of Formula (I). In some embodiments, the pharmaceutically acceptable solvate or hydrate is a salt of a compound of the present disclosure such as a compound of Formula (I).

### Process of Manufacturing

The compounds of the present disclosure, including those in Table 1 and the Examples, can be prepared by different ways with reactions known to one skilled in the art.

The present disclosure provides a process of manufacturing compounds of Formula (I):

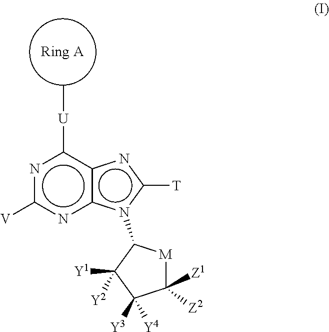

or pharmaceutical salts, hydrates, or solvates thereof, wherein U is a direct bond or is chosen from: (i) —O—, (ii) -alkoxy-, (iii) -(alkyl)O(alkyl)-, (iv) -alkyl-, (v) -alkenyl-, (vi) -alkyl-S— wherein alkyl is attached to Ring A, (vii) —SO₂NR¹— wherein the N is attached to Ring A, (viii) -alkyl-SO₂—NR¹— wherein the N is attached to Ring A, (ix) -alkyl-NR¹— wherein alkyl is attached to Ring A, (x) —NR¹—, (xi) —C(O)NR¹— wherein the N is attached to Ring A, (xii) —C(O)NR¹-alkyl- wherein the alkyl is attached to Ring A, and (xiii) —CO—; wherein Ring A, T, V, M, Y¹, Y², Y³, Y⁴, Z¹, and Z² are as defined herein.

The present disclosure provides a process of manufacturing compounds of Formula (Ia):

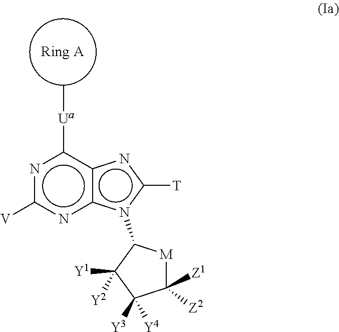

or pharmaceutical salts, hydrates, or solvates thereof, wherein U^{a} is chosen from: (i) —O—, (ii) -alkoxy-, and (iii) -(alkyl)O(alkyl)-; and wherein Ring A, T, V, M, Y¹, Y², Y³, Y⁴, Z¹, and Z² are as defined herein.

The present disclosure provides a process of manufacturing compounds of Formula (Ib):

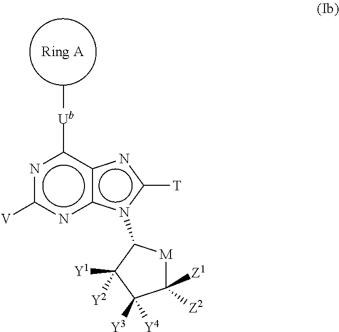

or pharmaceutical salts, hydrates, or solvates thereof, wherein U^{b} is: (i) -alkyl- or (ii) -alkenyl-; and wherein Ring A, T, V, M, Y¹, Y², Y³, Y⁴, Z¹, and Z² are as defined herein.

The present disclosure provides a process of manufacturing compounds of Formula (Ic):

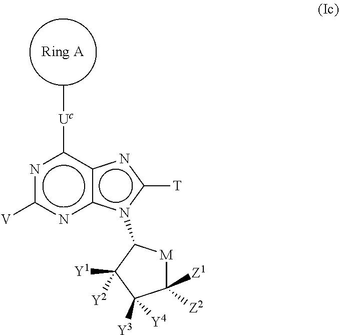

or pharmaceutical salts, hydrates, or solvates thereof, wherein U^{c} is chosen from: (i) -alkyl-NR¹— wherein alkyl is attached to Ring A, (ii) —NR¹—, (iii) —CO—, (iv) —C(O)NR¹— wherein the N is attached to Ring A, and (v) —C(O)NR¹-alkyl- wherein the alkyl is attached to Ring A; and wherein Ring A, T, V, M, Y¹, Y², Y³, Y⁴, Z¹, and Z² are as defined herein.

The present disclosure provides a process of manufacturing compounds of Formula (Id):

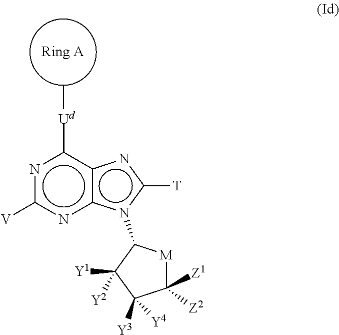

or pharmaceutical salts, hydrates, or solvates thereof, wherein U^{d} is chosen from: (i) -alkyl-S— wherein alkyl is attached to Ring A, (ii) —SO₂NR¹— wherein the N is attached to Ring A, and (iii) -alkyl-SO₂—NR¹— wherein the N is attached to Ring A; and wherein Ring A, T, V, M, Y¹, Y², Y³, Y⁴, Z¹, and Z² are as defined herein.

The present disclosure provides a process of manufacturing compounds of Formula (Ie):

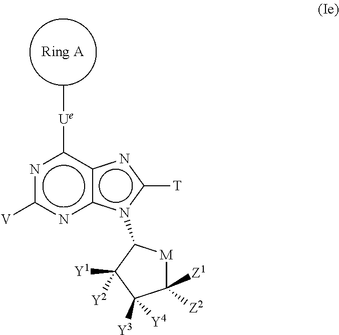

or pharmaceutical salts, hydrates, or solvates thereof, wherein U^{e} is a direct bond; and wherein Ring A, T, V, M, Y¹, Y², Y³, Y⁴, Z¹, and Z² are as defined herein.

### Methods of Use

In one aspect, provided herein is a method of modulating ENT transporters comprising contacting at least one ENT transporter with an effective amount of at least one compound of the present disclosure. Embodiments of the present disclosure provide a method for modulating at least one ENT transporters in a subject in need thereof, the method comprising administering to the subject an effective amount of at least one compound of the present disclosure. In some embodiments, the at least compound of the present disclosure modulates ENT1.

In one aspect, provided herein is a method of inhibiting ENT transporters in a patient need thereof, comprising administering to the patient an effective amount of at least one compound chosen from those disclosed herein. In some embodiments, the at least compound of the present disclosure inhibits ENT1.

Modulation (e.g., inhibition or activation) of ENT transporters can be assessed and demonstrated by a wide variety of ways known in the art. Kits and commercially available assays can be utilized for determining whether and to what degree ENT transporters, such as ENT1, has been modulated (e.g., inhibited or activated).

In some embodiments, a compound of the present disclosure modulates the activity of ENT transporters, including ENT1, by about 1%, 5%, 10%, 15%, 20%, 25%, 30%, 35%, 40%, 45%, 50%, 55%, 60%, 60%, 70%, 75%, 80%, 80%, 90%, 95%, or 100%. In some embodiments, a compound of the present disclosure modulates the activity of ENT transports, including ENT1, by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In some embodiments, provided herein is a method for targeting ENT transporters for degradation comprising contacting at least one ENT transporter with an effective amount of at least one compound of the present disclosure. In some embodiments, the ENT transporter is ENT1.

In one aspect, provided herein is a method of degrading ENT transporters comprising contacting at least one ENT transporter with an effective amount of at least one compound of the present disclosure. In some embodiments, the ENT transporter is ENT1.

Also provided in certain embodiments is a method for degrading ENT transporters in a subject in need thereof, the method comprising administering to the subject an effective amount of at least one compound of the present disclosure. In some embodiments, the ENT transporter is ENT1. In some embodiments, the compound of the present disclosure partially degrades an ENT transport, such as ENT1. In some embodiments, the compound of the present disclosure fully degrades an ENT transport, such as ENT1. Degradation of an ENT transport, such as ENT1, can be assessed and demonstrated by a wide variety of ways known in the art. Kits and commercially available assays, including cell-based assays, can be utilized for determining whether and to what degree an ENT transport, such as ENT1, has been degraded.

In some embodiments, a compound of the present disclosure degrades an ENT transport, such as ENT1, by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of the present disclosure degrades an ENT transport, such as ENT1, by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

The disclosure is further directed to the use of the compounds of the present disclosure as a medicament, i.e. for medical use. Thus, in one embodiment, the disclosure provides the use of the compounds of the present disclosure for the manufacture of a medicament. In one embodiment, the disclosure provides the use of the compounds of the present disclosure for the manufacture of a medicament.

In one aspect, the disclosure is further directed to the use of the compounds of the present disclosure as a medicament, e.g. for medical use. Thus, in one embodiment, the disclosure provides the use of the compounds of the present disclosure for the manufacture of a medicament.

In one embodiment, the disclosure provides the compounds of the present disclosure, for use in the treatment and/or prevention of proliferative disorders, including cancers. Thus, in one embodiment, the disclosure provides the use of the compounds of the present disclosure for the manufacture of a medicament for treating and/or preventing cancer.

In one embodiment, the disclosure also provides a method of treating cancer in a patient need thereof, comprising administering to the patient an effective amount of at least one compound chosen from those disclosed herein.

In some embodiments, the disclosure also provides for a method for delaying in patient the onset of cancer comprising administering to the patient an effective amount of at least one compound of the present disclosure.

Cancers include solid cancers and non-solid cancers, including benign and malignant solid tumors and benign and malignant non-solid tumors. The cancer may be metastatic or non-metastatic. The cancer may be may be familial or sporadic.

In one embodiment, the cancer to be treated according to the present disclosure is a solid cancer. As used herein, the term “solid cancer” encompasses any cancer that forms a discrete tumor mass, as opposed to cancers that diffusely infiltrate a tissue without forming a mass.

Non-limiting examples of solid tumors include, but are not limited to: biliary tract cancer, brain cancer (including glioblastomas and medulloblastomas), breast cancer, carcinoid, cervical cancer, choriocarcinoma, colon cancer, colorectal cancer, endometrial cancer, esophageal cancer, gastric cancer, glioma, head and neck cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, thyroid cancer (including thyroid adenocarcinoma and medullary carcinoma) and urothelial cancer.

In another embodiment, the cancer to be treated according to the present disclosure is a non-solid cancer. Examples of non-solid tumors include but are not limited to hematological neoplasms. As used herein, a “hematologic neoplasm” is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

In one embodiment, the cancer is chosen from breast, carcinoid, cervical, colorectal, endometrial, glioma, head and neck, liver, lung, melanoma, ovarian, pancreatic, prostate, renal, gastric, thyroid, and urothelial cancers.

In one embodiment, the cancer is breast cancer. In one embodiment, the cancer is carcinoid cancer. In one embodiment, the cancer is cervical cancer. In one embodiment, the cancer is colorectal cancer. In one embodiment, the cancer is endometrial cancer. In one embodiment, the cancer is glioma. In one embodiment, the cancer is head and neck cancer. In one embodiment, the cancer is liver cancer. In one embodiment, the cancer is lung cancer. In one embodiment, the cancer is melanoma. In one embodiment, the cancer is ovarian cancer. In one embodiment, the cancer is pancreatic cancer. In one embodiment, the cancer is prostate cancer. In one embodiment, the cancer is renal cancer. In one, the cancer is gastric cancer. In one embodiment, the cancer is thyroid cancer. In a specific embodiment, the cancer is urothelial cancer.

In one embodiment, the cancer is chosen from leukemia and multiple myeloma.

In some embodiments, administering at least one compound of the present disclosure to a subject that is predisposed to cancer prevents the subject from developing any symptoms of the cancer (such as tumor growth or metastasis). In some embodiments, administering at least one compound of the present disclosure to a subject that does not yet display symptoms of cancer prevents the subject from developing any symptoms of the cancer.

In some embodiments, provided herein is method of preventing a subject that is predisposed to cancer from developing cancer, the method comprising administering at least one compound of the present disclosure to the subject.

In some embodiments, the disclosure provides for a method of diminishing the extent of cancer in a patient, the method comprising administering at least one compound of the present disclosure to the patient. In some embodiments, the disclosure provides for a method of stabilizing cancer in a patient, the method comprising administering at least one compound of the present disclosure to the patient. In some embodiments, the method prevents the worsening of the cancer.

In another aspect, the disclosure provides for a method of delaying the occurrence or recurrence of cancer in a patient, the method comprising administering at least one compound of the present disclosure to the patient.

In some embodiments, the disclosure provides for a method of slowing the progression of cancer in a patient, the method comprising administering at least one compound of the present disclosure to the patient. In some embodiments, the method provides a partial remission of the cancer. In some embodiments, the method provides a total remission of the cancer. In some embodiments, the disclosure provides for a method of delaying the progression of cancer in a patient, the method comprising administering at least one compound of the present disclosure to the patient. In some embodiments, the method increases the quality of life of the patient having cancer. In some embodiments, the method prolongs survival of the patient having cancer.

In one embodiment, the patient receiving at least one compound of the present disclosure is being treated with at least one additional therapeutic agent in combination with the at least one compound of the present disclosure, or has received the at least one additional therapeutic agent within about fourteen days of administration of the at least one compound of the present disclosure. In one embodiment, the at least one additional therapeutic agent is administered up to fourteen days before the administration of the at least one compound of the present disclosure. In one embodiment, the at least one additional therapeutic agent is administered up to fourteen days after the administration of the at least one compound of the present disclosure. In one embodiment, the additional therapeutic agent comprises an adenosine receptor antagonist. Thus, in one embodiment, disclosed herein is a method of treating cancer in a patient need thereof, comprising administering to the patient a combination of at least one compound chosen from those disclosed herein and an adenosine receptor antagonist.

In one embodiment, the patient has previously received at least one prior therapeutic treatment, and has progressed subsequent to the administration of the at least one prior therapeutic treatment and prior to administration of least one compound of the present disclosure. In one embodiment, the prior therapeutic treatment is chosen from chemotherapy, immunotherapy, radiation therapy, stem cell transplant, hormone therapy, and surgery.

In one embodiment, the at least one compound of the present disclosure is administered prior to, concomitant with, or subsequent to administration of the additional therapeutic agent, such as an adenosine receptor antagonist. In one embodiment, the disclosure provides for a method of decreasing the dose of the additional therapeutic agent, the method comprising administering at least one compound of the present disclosure to the patient.

In some embodiments, provided herein is a method of enhancing the effect of the additional therapeutic agent, the method comprising administering at least one compound of the present disclosure to the patient.

In one embodiment, provided herein is a method of treating cancer in a patient need thereof, comprising administering to the patient at least one compound chosen from compounds of Formula (I) and Table 1, and an adenosine receptor antagonist. In some embodiments, the disclosure also provides for a method for delaying in patient the onset of cancer comprising administering to the patient at least one compound chosen from compounds of Formula (I) and Table 1, and an adenosine receptor antagonist. In some embodiments, provided herein is method of preventing a subject that is predisposed to cancer from developing cancer, the method comprising administering to the patient at least one compound chosen from compounds of Formula (I) and Table 1, and an adenosine receptor antagonist.

In one embodiment, the additional therapeutic agent comprises an adenosine receptor antagonist.

In one embodiment, the adenosine receptor antagonist is an antagonist of A1 receptor, A2A receptor, A2B receptor, A3 receptor or of a combination thereof. In one embodiment, the adenosine receptor antagonist is an antagonist of A2A receptor, A2B receptor or of a combination thereof. In one embodiment, the adenosine receptor antagonist is an A2A and/or A2B receptor antagonist.

In one embodiment, the adenosine receptor antagonist is an antagonist of A2A receptor, A2B receptor or of a combination thereof. In one embodiment, the adenosine receptor antagonist is an A2A or A2B receptor antagonist.

In one embodiment, the adenosine receptor antagonist is an antagonist which is selective of A2A receptor with respect to other adenosine receptors. In one embodiment, the adenosine receptor antagonist is an antagonist which is selective of A2A receptor with respect to A2B receptor.

In one embodiment, the adenosine receptor antagonist is an antagonist which is selective of A2B receptor with respect to other adenosine receptors. In one embodiment, the adenosine receptor antagonist is an antagonist which is selective of A2B receptor with respect to A2A receptor.

In one embodiment, a combination is disclosed herein comprises at least one A2A receptor antagonist and at least one compound of the present disclosure. Examples of A2AR antagonists include: Preladenant (SCH-420,814), Vipadenant (BIIB-014), Tozadenant (SYK-115), ATL-444, Istradefylline (KW-6002), MSX-3, SCH-58261, SCH-412,348, SCH-442,416, ST-1535, Caffeine, VER-6623, VER-6947, VER-7835, ZM-241,385, theophylline, imaradenant, etrumadenant, taminadenant, ciforadenant, INCB106385, DZD2269, CS3005, EXS21546, TT-10, TT-4, ILB2109, M1069, and CPI-935. It also includes A2AR antagonists disclosed in WO 2018/178338, WO 2011/121418, WO 2009/156737, WO 2011/095626 or WO 2018/136700, the content of which is herein incorporated by reference.

In one embodiment, the A2AR antagonist is a thiocarbamate derivative, especially a thiocarbamate derivative as those disclosed in WO 2018/178338. In some embodiments, the A2AR antagonist is a thiocarbamate derivative of Formula (III) according to WO 2018/178338:

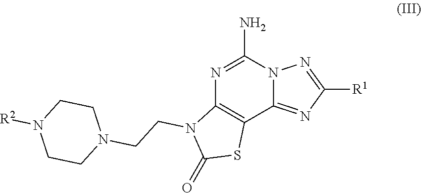

or a pharmaceutically acceptable salt, hydrate, or solvate thereof, wherein:

R¹ represents 5- or 6-membered heteroaryl or 5- or 6-membered aryl, wherein heteroaryl or aryl groups are optionally substituted by one or more substituent selected from C₁₋₆ alkyl (such as methyl) and halo (such as fluoro or chloro); for example, in one embodiment, R¹ represents 5-membered heteroaryl; in another embodiment, R¹ represents furyl;
R² represents 6-membered aryl or 6-membered heteroaryl,
wherein heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl alkylsulfonyl and alkylsulfonealkyl;
or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, the A2AR antagonists of Formula (III) are of Formula (IIIa):

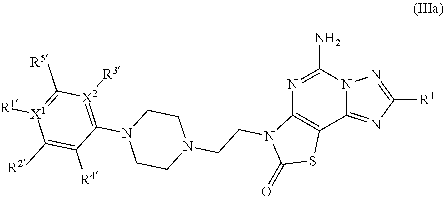

or a pharmaceutically acceptable salt, hydrate, or solvate thereof, wherein:
R¹ represents 5- or 6-membered heteroaryl or 5- or 6-membered aryl, wherein heteroaryl or aryl groups are optionally substituted by one or more substituent selected from C₁-C₆ alkyl (such as methyl) and halo (such as fluoro or chloro); for example, in one embodiment,
R¹ represents 5-membered heteroaryl; in another embodiment, R¹ represents furyl;
X¹ and X² represent each independently C or N;
R^{1′} is absent when X¹ is N; or when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
or R^{1′} and R^{2′} form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
R^{3′} is absent when X² is N; or when X² is C, R^{3′} represents H or halo, such as H or F;
R^{4′} represents H or halo, such as H or F; and
R^{5′} represents H or halo, such as H or F.

In one embodiment, the A2AR antagonists of Formula (IIIa) are those of Formula (IIIa-1):

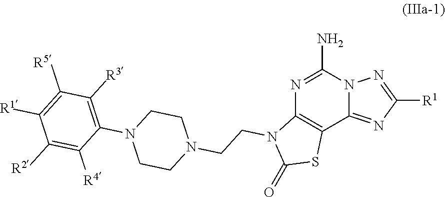

or a pharmaceutically acceptable salt, hydrate, or solvate thereof, wherein R¹, R^{1′}, R^{2′}, R^{3′}, R^{4′} and R^{5′} are as defined in Formula (IIIa).

In one embodiment, the A2AR antagonists of Formula (IIIa-1) are those of Formula (IIIa-1a):

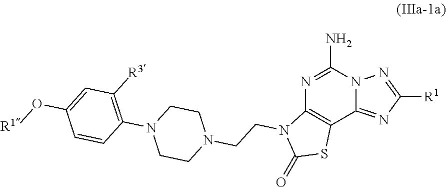

or a pharmaceutically acceptable salt, hydrate, or solvate thereof, wherein:
R¹ and R^{3′} are as defined in Formula (IIIa); and
R^{1″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, the A2AR antagonists of Formula (IIIa-1) are those of Formula (IIIa-1b):

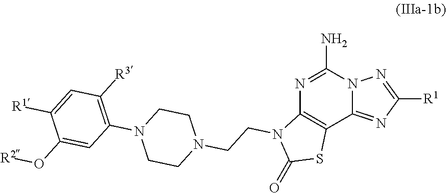

or a pharmaceutically acceptable salt, hydrate, or solvate thereof, wherein:
R¹ and R^{3′} are as defined in Formula (IIIa);
R^{1′} represents H or halo, such as H or F; and
R^{2″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, the A2AR antagonists of Formula (IIIa-1) are those of Formula (IIIa-1c) or (IIIa-1d):

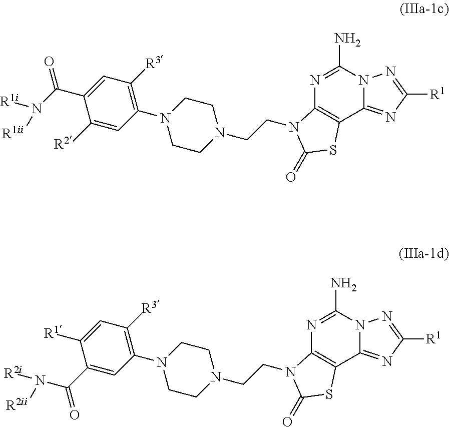

or a pharmaceutically acceptable salt, hydrate, or solvate thereof, wherein:
R¹ and R^{3′} are as defined in Formula (IIIa);
R^{1′} represents H or halo, such as H or F;
R^{2′} represents H or halo, such as H or F;
Ru and Rut represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl; and
R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

In one embodiment, the A2AR antagonists of Formula (IIIa) are those of Formulae (IIIa-2) or (IIIa-3):

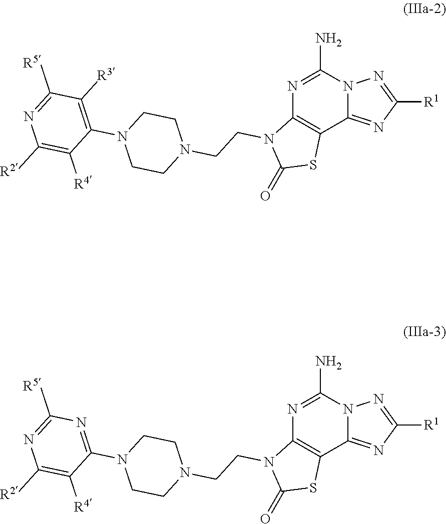

or a pharmaceutically acceptable salt, hydrate, or solvate thereof, wherein R¹, R^{2′}, R^{3′}, R^{4′} and R^{5′} are as defined in Formula (IIIa).

In certain embodiments, the A2AR antagonists of Formula (III) are chosen from those listed hereafter, or a pharmaceutically acceptable salt, hydrate, or solvate thereof:
3-(2-(4-(4-((1H-1,2,3-triazolo-4yl)methoxy-2fluorophenyl)piperazine-1-yl)ethyl)-5-amino-(8-(furan-2-yl)thiazolo[5,4-e][1,2,4]triazolo[1,5-c]pyrimidine-2(3H)-one
5-((4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)methyl)-1,3,4-oxadiazol-2(3H)-one
5-amino-3-(2-(4-(3-fluoropyridin-4-yl)piperazin-1-yl)ethyl)-8-(furan-2-yl)thiazolo[5,4-e][1,2,4]triazolo[1,5-c]pyrimidin-2(3H)-one
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)acetamide
(S)-5-amino-3-(2-(4-(2-fluoro-4-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(2-fluoro-4-(2-(methylsulfinyl)ethoxy)phenyl)-piperazin-1-yl)ethyl)-8-(furan-2-yl)thiazolo[5,4-e][1,2,4]triazolo[1,5-c]pyrimidin-2(3H)-one
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one
5-amino-8-(furan-2-yl)-3-(2-(4-(4-(2-hydroxyethoxy) phenyl)piperazin-1-yl)ethyl)thiazolo[5,4-e][1,2,4]triazolo[1,5-c]pyrimidin-2(3H)-one
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)phenoxy)acetic acid
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)phenoxy)acetamide
5-amino-3-(2-(4-(4-(2,3-dihydroxypropoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(4-(2-aminoethoxy)phenyl)piperazin-1-yl)ethyl)-8-(furan-2-yl)thiazolo[5,4-e][1,2,4]triazolo[1,5-c]pyrimidin-2(3H)-one
4-(4-(2-(5-amino-8-(furan-2-yl)-2-oxothiazolo[5,4-e][1,2,4]triazolo[1,5-c]pyrimidin-3(2H)-yl) ethyl)piperazin-1-yl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-methylbenzamide
5-amino-8-(furan-2-yl)-3-(2-(4-(4-(2-morpholinoethoxy)phenyl)piperazin-1-yl)ethyl)thiazolo[5,4-e][1,2,4]triazolo[1,5-c]pyrimidin-2(3H)-one
5-amino-3-(2-(4-(4-(2-(dimethylamino)ethoxy)phenyl)piperazin-1-yl)ethyl)-8-(furan-2-yl)thiazolo[5,4-e][1,2,4]triazolo[1,5-c]pyrimidin-2(3H)-one
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzenesulfonamide
4-(4-(2-(5-amino-8-(furan-2-yl)-2-oxothiazolo[5,4-e][1,2,4]triazolo[1,5-c]pyrimidin-3(2H)-yl)ethyl) piperazin-1-yl)-N-methylbenzenesulfonamide
5-amino-8-(furan-2-yl)-3-(2-(4-(4-(methylsulfonyl)phenyl)piperazin-1-yl)ethyl)thiazolo[5,4-e][1,2,4]triazolo[1,5-c]pyrimidin-2(3H)-one
5-amino-8-(furan-2-yl)-3-(2-(4-(4-(methylsulfinyl)phenyl)piperazin-1-yl)ethyl)thiazolo[5,4-e][1,2,4]triazolo[1,5-c]pyrimidin-2(3H)-one
3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzamide
5-amino-8-(furan-2-yl)-3-(2-(4-(3-(2-hydroxyethoxy) phenyl)piperazin-1-yl)ethyl)thiazolo[5,4-e][1,2,4]triazolo[1,5-c]pyrimidin-2(3H)-one
5-amino-3-(2-(4-(2-fluoro-4-(2-oxo-2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(piperidin-4-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(piperazine-1-carbonyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(piperazin-1-ylsulfonyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(methylsulfonyl)phenyl) piperazin-1-yl)ethyl)-8-(furan-2-yl)thiazolo[5,4-e][1,2,4]triazolo[1,5-c]pyrimidin-2(3H)-one
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-aminoethyl)-3-fluorobenzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylamino)ethyl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluorobenzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-hydroxyethyl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-3-fluorobenzamide
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)acetic acid
2-(4-(4-(2-(5-amino-8-(furan-2-yl)-2-oxothiazolo[5,4-e][1,2,4]triazolo[1,5-c]pyrimidin-3(2H)-yl)ethyl) piperazin-1-yl)-3,5-difluorophenoxy) acetic acid
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid
(S)-2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-2-methylpropanoic acid
3-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenyl)propanoic acid
4-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)butanoic acid
2-(3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,6-difluorophenoxy) acetic acid
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetic acid
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorobenzoic acid
2-((2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)amino)acetamide
2-((2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)(methyl)amino)acetamide
5-amino-3-(2-(4-(2-fluoro-4-(piperidin-4-yloxy)phenyl)piperazin-1-yl) ethyl)-8-(furan-2-yl) thiazolo[5,4-e][1,2,4]triazolo[1,5-c]pyrimidin-2(3H)-one
5-amino-3-(2-(4-(2-fluoro-4-(pyrrolidin-3-yloxy)phenyl)piperazin-1-yl)ethyl)-8-(furan-2-yl)thiazolo[5,4-e][1,2,4]triazolo[1,5-c]pyrimidin-2(3H)-one
3-(2-(4-(4-((1H-1,2,4-triazol-3-yl)methoxy)-2-fluorophenyl)piperazin-1-yl)ethyl)-5-amino-8-(furan-2-yl)thiazolo[5,4-e][1,2,4]triazolo[1,5-c]pyrimidin-2(3H)-one
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-(methylamino)ethyl) acetamide
2-(4-(4-(2-(5-amino-8-(furan-2-yl)-2-oxothiazolo[5,4-e][1,2,4]triazolo[1,5-c]pyrimidin-3(2H)-yl) ethyl)piperazin-1-yl)-3-fluorophenoxy)-N-(2-(dimethylamino)ethyl) acetamide
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-aminoethyl)acetamide
(R)-2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid
2-(4-(4-(2-(5-amino-8-(furan-2-yl)-2-oxothiazolo[5,4-e][1,2,4]triazolo[1,5-c]pyrimidin-3(2H)-yl) ethyl)piperazin-1-yl)-3-fluorophenoxy)acetamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-methyl-N-(2-(methylamino)ethyl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluoro-N-methylbenzamide
(R)-4-(4-(2-(5-amino-8-(furan-2-yl)-2-oxothiazolo[5,4-e][1,2,4]triazolo[1,5-c]pyrimidin-3(2H)-yl) ethyl)piperazin-1-yl)-N-(1-(dimethylamino) propan-2-yl)-3-fluorobenzamide
2-(4-(4-(2-(5-amino-8-(furan-2-yl)-2-oxothiazolo[5,4-e][1,2,4]triazolo[1,5-c]pyrimidin-3(2H)-yl) ethyl)piperazin-1-yl)-3-fluorophenoxy)-N-methyl-N-(2-(methylamino)ethyl) acetamide
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-2-methylpropanoic acid
(S)-2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) propanoic acid
(R)-2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) propanoic acid
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-(methylamino)ethyl) acetamide
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-(dimethylamino)ethyl) acetamide
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluoro-N-methylbenzamide
4-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) butanoic acid
3-(2-(4-(5-((1H-tetrazol-5-yl)methoxy)-2,4-difluorophenyl)piperazin-1-yl)ethyl)-5-amino-8-(furan-2-yl)thiazolo[5,4-e][1,2,4]triazolo[1,5-c]pyrimidin-2(3H)-one
5-amino-3-(2-(4-(2-fluoro-4-((1-methyl-1H-1,2,4-triazol-3-yl)methoxy) phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-((1-methyl-1H-1,2,4-triazol-3-yl) methoxy)phenyl)piperazin-1-yl)ethyl)-8-(furan-2-yl)thiazolo[5,4-e][1,2,4]triazolo[1,5-c]pyrimidin-2(3H)-one
4-(4-(2-(5-amino-8-(furan-2-yl)-2-oxothiazolo[5,4-e][1,2,4]triazolo[1,5-c]pyrimidin-3(2H)-yl) ethyl)piperazin-1-yl)-3-fluoro-N-(2-(methyl (oxetan-3-yl)amino)ethyl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-((2-hydroxyethyl)amino)ethyl)benzamide
2-amino-N-(2-(4-(4-(2-(5-amino-8-(furan-2-yl)-2-oxothiazolo[5,4-e][1,2,4]triazolo[1,5-c]pyrimidin-3(2H)-yl) ethyl)piperazin-1-yl)-3-fluorophenoxy)ethyl) acetamide
(S)-2-amino-N-(2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)-3-methylbutanamide
ethyl 2-(5-(4-(2-(5-amino-8-(furan-2-yl)-2-oxothiazolo[5,4-e][1,2,4]triazolo[1,5-c]pyrimidin-3(2H)-yl) ethyl)piperazin-1-yl)-2,4-difluorophenoxy)acetate
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetonitrile
5-amino-8-(furan-2-yl)-3-(2-(4-(pyridin-4-yl) piperazin-1-yl)ethyl)thiazolo[5,4-e][1,2,4]triazolo[1,5-c]pyrimidin-2(3H)-one
5-amino-8-(furan-2-yl)-3-(2-(4-(pyrimidin-4-yl)piperazin-1-yl)ethyl)thiazolo[5,4-e][1,2,4]triazolo[1,5-c]pyrimidin-2(3H)-one
5-amino-3-(2-(4-(2,4-difluoro-5-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(6-fluoro-2-oxoindolin-5-yl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(S-methylsulfonimidoyl)phenyl)piperazin-1-yl)ethyl)-8-(furan-2-yl)thiazolo[5,4-e][1,2,4]triazolo[1,5-c]pyrimidin-2(3H)-one
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluorobenzamide
5-amino-3-(2-(4-(5-fluoro-2-methylpyridin-4-yl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3R,4R)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3S,4S)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(2-hydroxy-2-methylpropoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(2-hydroxypropan-2-yl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(3,3,3-trifluoro-2-hydroxypropoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-5-(2-hydroxyethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-morpholinoethyl)acetamide
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(morpholin-3-ylmethyl)benzamide
5-amino-3-(2-(4-(2-fluoro-4-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3R,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-morpholinoethyl)acetamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-morpholinoethyl)benzamide
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(morpholin-3-ylmethyl)benzamide
5-amino-3-(2-(4-(4-(azetidin-3-yloxy)phenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide
5-amino-3-(2-(4-(2,4-difluoro-5-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2,4-difluoro-5-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one
(S)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide
(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide
5-amino-3-(2-(4-(2-fluoro-4-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one
5-amino-3-(2-(4-(2-fluoro-4-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one
(R)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide
5-amino-3-(2-(4-(4-(azetidin-3-yloxy)-2-fluorophenyl)piperazin-1-yl)ethyl)-8-(furan-2-yl)thiazolo[5,4-e][1,2,4]triazolo[1,5-c]pyrimidin-2(3H)-one
5-amino-3-(2-(4-(5-(azetidin-3-yloxy)-2,4-difluorophenyl)piperazin-1-yl)ethyl)-8-(furan-2-yl)thiazolo[5,4-e][1,2,4]triazolo[1,5-c]pyrimidin-2(3H)-one
(S)-5-amino-3-(2-(4-(2,4-difluoro-5-(3-(methylsulfinyl)propoxy)phenyl)piperazin-1-yl)ethyl)-8-(furan-2-yl)thiazolo[5,4-e][1,2,4]triazolo[1,5-c]pyrimidin-2(3H)-one.

In one embodiment, the A2AR antagonist of Formula (III) is selected from:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.

In one embodiment, the A2AR antagonist of Formula (III) is selected from:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.

In one embodiment, the A2AR antagonist of Formula (III) is (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.

In one embodiment, the A2AR antagonist of Formula (III) is (−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.

In one embodiment, the adenosine receptor antagonist is (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one, or a pharmaceutically acceptable salt, hydrate, or solvate thereof.

The embodiments relative to salts, hydrates, solvates, enantiomers, isomers (including optical, geometric and tautomeric isomers), polymorphs, multi-component complexes, liquid crystals, prodrugs and isotopically-labeled ENT inhibitors of the invention also apply to the A2AR antagonists Formula (III) and subformula thereof detailed above.

In another embodiment, the A2AR antagonist is an A2AR antagonist disclosed in WO 2011/121418, including the compound of example 1 (5-bromo-2,6-di-(1H-pyrazol-1-yl)pyrimidin-4-amine, also known as NIR178):

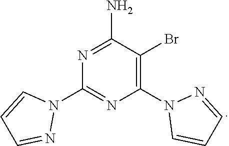 [SMILES: Nc1nc(-n2cccn2)nc(-n2cccn2)c1Br]

In another embodiment, the A2AR antagonist is an A2AR antagonist disclosed in WO 2009/156737, including the compound of example 1S ((S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine, also known as CPI-444):

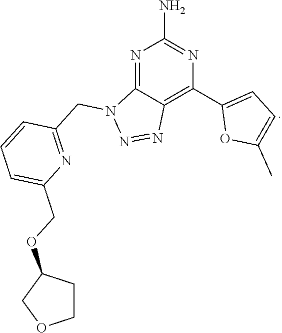 [SMILES: Cc1ccc(-c2nc(N)nc3c2nnn3Cc2cccc(CO[C@H]3CCOC3)n2)o1]

In another embodiment, the A2AR antagonist is an A2AR antagonist disclosed in WO 2011/095626, including the compound (cxiv) (6-(2-chloro-6-methylpyridin-4-yl)-5-(4-fluorophenyl)-1,2,4-triazin-3-amine, also known as AZD4635):

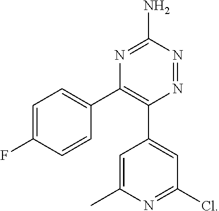 [SMILES: Cc1cc(-c2nnc(N)nc2-c2ccc(F)cc2)cc(Cl)n1]

In another embodiment, the A2AR antagonist is an A2AR antagonist disclosed in WO 2018/136700, including the compound of example 1 (3-(2-amino-6-(1-((6-(2-hydroxypropan-2-yl)pyridin-2-yl)methyl)-1H-1,2,3-triazol-4-yl)pyrimidin-4-yl)-2-methylbenzonitrile, also known as AB928):

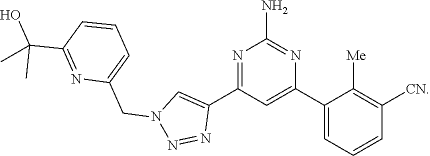 [SMILES: Cc1c(C#N)cccc1-c1cc(-c2cn(Cc3cccc(C(C)(C)O)n3)nn2)nc(N)n1]

In another embodiment, the A2AR antagonist is Preladenant (2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine, also known as SCH-420,814):

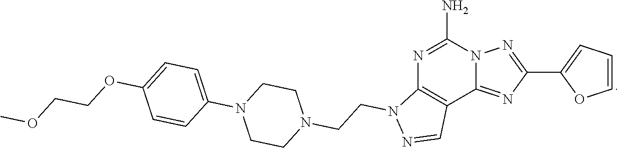 [SMILES: COCCOc1ccc(N2CCN(CCn3ncc4c3nc(N)n3nc(-c5ccco5)nc43)CC2)cc1]

In another embodiment, the A2AR antagonist is Vipadenant (3-(4-amino-3-methylbenzyl)-7-(2-furyl)-3H-(1,2,3)triazolo(4,5-d)pyrimidine-5-amine, also known as BIIB-014):

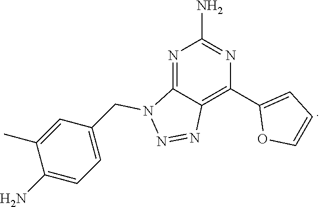 [SMILES: Cc1cc(Cn2nnc3c(-c4ccco4)nc(N)nc32)ccc1N]

In another embodiment, the A2AR antagonist is Tozadenant (4-hydroxy-N-(4-methoxy-7-morpholinobenzo[d]thiazol-2-yl)-4-methylpiperidine-1-carboxamide, also known as SYK-115):

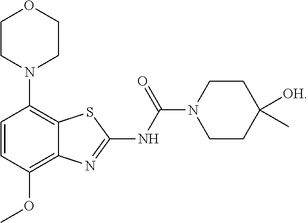 [SMILES: COc1ccc(N2CCOCC2)c2sc(NC(=O)N3CCC(C)(O)CC3)nc12]

In one embodiment, the adenosine receptor antagonist is chosen from: (i) 5-bromo-2,6-di-(1H-pyrazol-1-yl)pyrimidin-4-amine, (ii) (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine, (iii) 6-(2-chloro-6-methylpyridin-4-yl)-5-(4-fluorophenyl)-1,2,4-triazin-3-amine, (iv) 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile, (v) 2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine, (vi) 3-(4-amino-3-methylbenzyl)-7-(2-furyl)-3H-(1,2,3)triazolo(4,5-d)pyrimidine-5-amine, and (vii) 4-hydroxy-N-(4-methoxy-7-morpholinobenzo[d]thiazol-2-yl)-4-methylpiperidine-1-carboxamide.

In one embodiment, the adenosine receptor antagonist is 5-bromo-2,6-di-(1H-pyrazol-1-yl)pyrimidin-4-amine. In one embodiment, the adenosine receptor antagonist is (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine. In one embodiment, the adenosine receptor antagonist is 6-(2-chloro-6-methylpyridin-4-yl)-5-(4-fluorophenyl)-1,2,4-triazin-3-amine. In one embodiment, the adenosine receptor antagonist is 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile.

### Pharmaceutical Compositions and Routes of Administration

In some embodiments, the disclosure provides pharmaceutical compositions comprising at least one compound of the present disclosure, or a pharmaceutically acceptable salt, hydrate, and solvate thereof, and at least one pharmaceutically acceptable excipient.

In some embodiments, the disclosure provides a medicament comprising at least one compound of the present disclosure, or a pharmaceutically acceptable salt, hydrate, and solvate thereof, as active ingredient.

By means of non-limiting examples, the pharmaceutical compositions disclosed herein may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

In some embodiments, such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propyl-hydroxybenzoates, talc, magnesium stearate, edible oils, vegetable oils and mineral oils or suitable mixtures thereof. The pharmaceutical compositions can optionally contain other substances that are commonly used in pharmaceutical compositions, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein.

In some embodiments, the pharmaceutical compositions of the present disclosure are in a unit dosage form, and may be suitably packaged, for example in a box, blister, vial, bottle, sachet, ampoule or in any other suitable single-dose or multi-dose holder or container (which may be properly labeled), optionally with one or more leaflets containing product information and/or instructions for use.

Depending on the condition to be prevented or treated and the route of administration, the active compound may be administered as a single daily dose, divided over one or more daily doses, or essentially continuously, e.g. using a drip infusion.

The effective amount of the compounds of the present disclosure in the pharmaceutical composition may be at a level that will exercise the desired effect; for example, about 0.001 mg/kg of a subject's body weight to about 10 mg/kg of a subject's body weight in unit dosage for both oral and parenteral administration.

The dose of a compound of the present disclosure to be administered to a subject is rather widely variable and can be subject to the judgment of a health-care practitioner. In general, the compounds disclosed herein can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In one embodiment, the dose is about 0.001 mg/kg of a subject's body weight to about 5 mg/kg of a subject's body weight, about 0.01 mg/kg of a subject's body weight to about 5 mg/kg of a subject's body weight, about 0.05 mg/kg of a subject's body weight to about 1 mg/kg of a subject's body weight, about 0.1 mg/kg of a subject's body weight to about 0.75 mg/kg of a subject's body weight or about 0.25 mg/kg of a subject's body weight to about 0.5 mg/kg of a subject's body weight. In one embodiment, one dose is given per day. In any given case, the amount of the compound of the present disclosure administered will depend on such factors as the solubility of the active component, the formulation used and the route of administration.

In some embodiments, at least one compound of the present disclosure is administered to a subject at a dose of about 0.01 mg/day to about 750 mg/day, about 0.1 mg/day to about 375 mg/day, about 0.1 mg/day to about 150 mg/day, about 0.1 mg/day to about 75 mg/day, about 0.1 mg/day to about 50 mg/day, about 0.1 mg/day to about 25 mg/day, or about 0.1 mg/day to about 10 mg/day.

In another embodiment, the disclosure provides unit dosage formulations that comprise between about 0.1 mg and 500 mg, about 1 mg and 250 mg, about 1 mg and about 100 mg, about 1 mg and about 50 mg, about 1 mg and about 25 mg, or between about 1 mg and about 10 mg of at least one compound of the present disclosure.

In some embodiments, the disclosure provides unit dosage formulations comprising about 0.1 mg or 100 mg of at least one compound of the present disclosure.

In another embodiment, the disclosure provides unit dosage formulations that comprise 0.5 mg, 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of at least one compound of the present disclosure.

A compound of the present disclosure can be administered once, twice, three, four or more times daily. In one embodiment, doses of 100 mg or less are administered as a once daily dose and doses of more than 100 mg are administered twice daily in an amount equal to one half of the total daily dose.

A compound of the present disclosure can be administered orally for reasons of convenience. In one embodiment, when administered orally, a compound of the present disclosure is administered with a meal and water. In another embodiment, the compound of the present disclosure is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, the disclosure provides capsules containing at least one compound of the present disclosure without any excipients.

In general, all of the compositions are prepared according to known methods in pharmaceutical chemistry. Capsules can be prepared by mixing at least one compound of the present disclosure with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of the present disclosure as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound the present disclosure can be delayed or prolonged by proper formulation. For example, a slowly soluble pellet of the compound of the present disclosure can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of the present disclosure in oily or emulsified vehicles that allow it to disperse slowly in the serum.

In one embodiment, the pharmaceutical composition according to the disclosure, further comprises an adenosine receptor antagonist. In one embodiment, the adenosine receptor antagonist is an A2A or A2B receptor antagonist. In one embodiment, the adenosine receptor antagonist is chosen from any adenosine receptor antagonists described above. In one embodiment, the adenosine receptor antagonist is chosen from: (i) 5-bromo-2,6-di-(1H-pyrazol-1-yl)pyrimidin-4-amine, (ii) (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine, (iii) 6-(2-chloro-6-methylpyridin-4-yl)-5-(4-fluorophenyl)-1,2,4-triazin-3-amine, (iv) 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile, (v) 2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine, (vi) 3-(4-amino-3-methylbenzyl)-7-(2-furyl)-3H-(1,2,3)triazolo(4,5-d)pyrimidine-5-amine, and (vii) 4-hydroxy-N-(4-methoxy-7-morpholinobenzo[d]thiazol-2-yl)-4-methylpiperidine-1-carboxamide.

In one embodiment, the pharmaceutical composition comprises a combination of active ingredients. In one embodiments, the pharmaceutical composition comprises: (i) an effective amount of at least one compound of the present disclosure; (ii) an effective amount of an adenosine receptor antagonist; and (iii) an pharmaceutically acceptable excipient. In one embodiments, the pharmaceutical composition comprises: (i) an effective amount of at least one compound of the present disclosure; (ii) an effective amount of any adenosine receptor antagonists described above; and (iii) an pharmaceutically acceptable excipient. In one embodiment, the adenosine receptor antagonist is chosen from: (i) 5-bromo-2,6-di-(1H-pyrazol-1-yl)pyrimidin-4-amine, (ii) (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine, (iii) 6-(2-chloro-6-methylpyridin-4-yl)-5-(4-fluorophenyl)-1,2,4-triazin-3-amine, (iv) 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile, (v) 2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine, (vi) 3-(4-amino-3-methylbenzyl)-7-(2-furyl)-3H-(1,2,3)triazolo(4,5-d)pyrimidine-5-amine, and (vii) 4-hydroxy-N-(4-methoxy-7-morpholinobenzo[d]thiazol-2-yl)-4-methylpiperidine-1-carboxamide.

### Kits

In one aspect, the disclosure provides a kit of parts comprising: (i) a first part comprising an effective amount of at least one compound chosen from those disclosed herein; and (ii) a second part comprising an effective amount of an adenosine receptor antagonist. In one embodiment, the adenosine receptor antagonist is any of the adenosine receptor agonists described above. In one embodiment, the adenosine receptor antagonist is chosen from: (i) 5-bromo-2,6-di-(1H-pyrazol-1-yl)pyrimidin-4-amine, (ii) (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine, (iii) 6-(2-chloro-6-methylpyridin-4-yl)-5-(4-fluorophenyl)-1,2,4-triazin-3-amine, (iv) 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile, (v) 2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine, (vi) 3-(4-amino-3-methylbenzyl)-7-(2-furyl)-3H-(1,2,3)triazolo(4,5-d)pyrimidine-5-amine, and (vii) 4-hydroxy-N-(4-methoxy-7-morpholinobenzo[d]thiazol-2-yl)-4-methylpiperidine-1-carboxamide.

Depending on the ENT inhibitor and adenosine receptor antagonist, the first and second parts of the kit may be under the form of pharmaceutical compositions.

In one embodiment, the kit of parts of the present disclosure further comprises an additional therapeutic agent.

In one embodiment, the disclosure provides for a kit of parts as described herein for use in the treatment and/or prevention of cancer. The disclosure further provides for a use of the kit of parts as described herein for the manufacture of a medicament for treating and/or preventing cancer. The disclosure further provides a method of treating of cancer, which comprises administering to a patient in need thereof a kit of parts as described herein.

### Enumerated Embodiments

Embodiment 1. A compound of Formula (I):

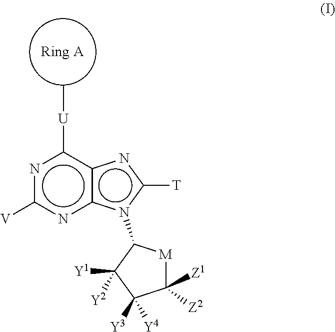

or a pharmaceutically acceptable salt, hydrate, or solvate thereof, wherein:
Ring A is chosen from optionally substituted aryl, optionally substituted heteroaryl containing at least one N, O, or S atom, optionally substituted heterocyclyl containing at least one N or O, and optionally substituted cycloalkyl;
U is a direct bond or is chosen from: (i) —O—, (ii) -alkoxy-, (iii) -(alkyl)O(alkyl)-, (iv) -alkyl-, (v) -alkenyl-, (vi) -alkyl-S— wherein the alkyl is attached to Ring A, (vii) —SO₂NR¹— wherein the N is attached to Ring A, (viii) -alkyl-SO₂—NR¹— wherein the N is attached to Ring A, (ix) -alkyl-NR¹— wherein the alkyl is attached to Ring A, (x) —NR¹—, (xi) —C(O)NR¹— wherein the N is attached to Ring A, (xii) —C(O)NR¹-alkyl- wherein the alkyl is attached to Ring A, and (xiii) —CO—;
T is chosen from —H, —OH, —O(alkyl)(aryl), optionally substituted heteroaryl that contains at least one N atom, —C(O)NR¹(cycloalkyl), and optionally substituted amine;
V is chosen from —H, -halo, —OH, -alkyl, and -alkoxy;
M is —O— or —C(R²)₂—;
Y¹ and Y² are each independently chosen from —H, —OH, and -halo;
Y³ is —OH or —H;
Y⁴ is chosen from —H, -halo, —OH, and -alkyl optionally substituted with 1, 2, or 3 groups independently chosen from -halo and —OH;
Z¹ is chosen from —H, -halo, —OH, and -alkyl optionally substituted with 1, 2, or 3 groups independently chosen from —OH and -halo;
Z² is chosen from —H, -alkyl, -alkenyl, —C(O)NHR¹, —C(O)NR¹(alkyl), and —C(O)O(alkyl), wherein each alkyl and alkenyl group is optionally substituted;
each R¹ is independently chosen from —H and -alkyl; and
each R² is independently chosen from —H, -alkyl, and -cycloalkyl;
on the condition that:
(a) when U is chosen from (i) —O—, (ii) -alkoxy-, and (iii) -(alkyl)O(alkyl)-, then:
at least one of Y¹, Y² or Y³ is —OH; and
when Z² is alkyl, it is not substituted with a phosphonate group or a protected alcohol group; and
with the proviso that the compound is not

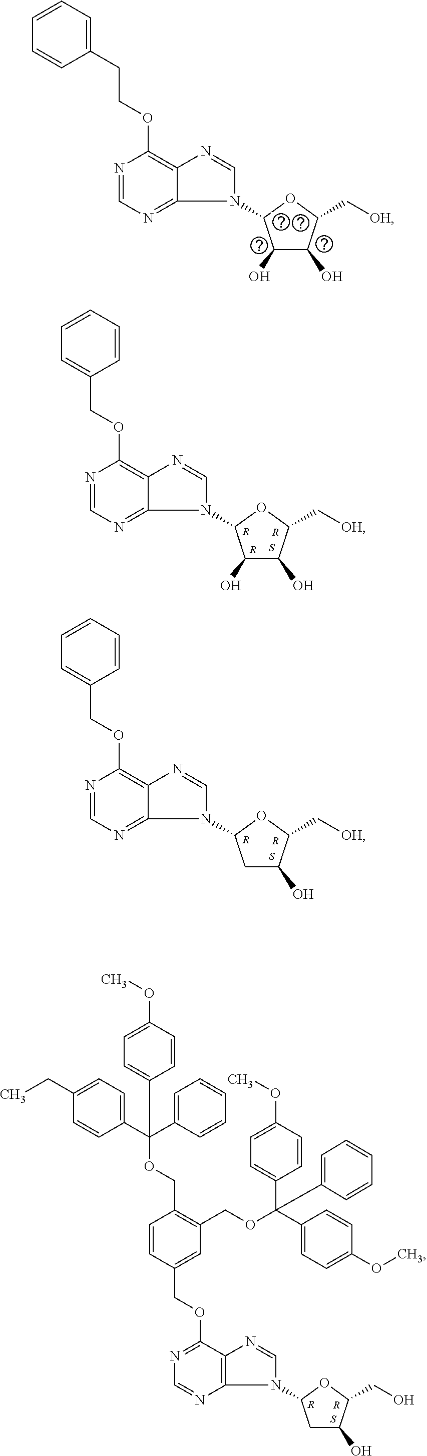

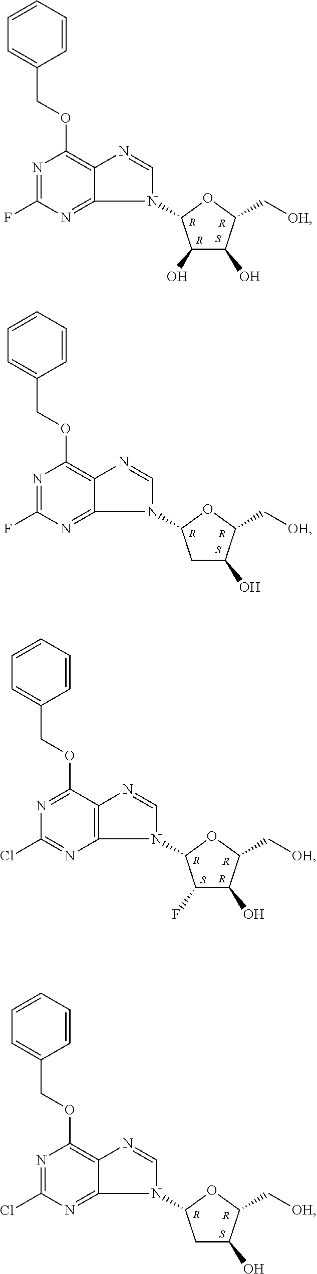

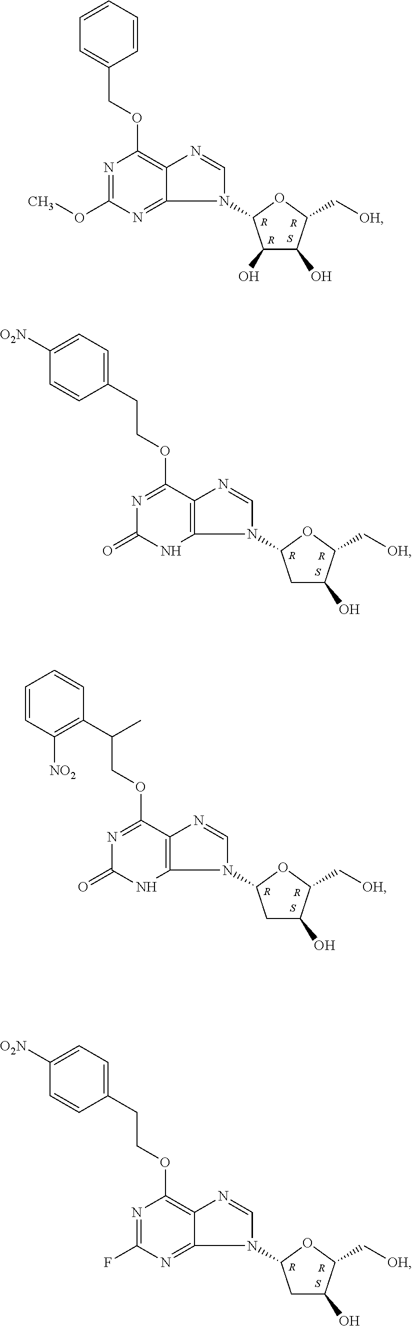

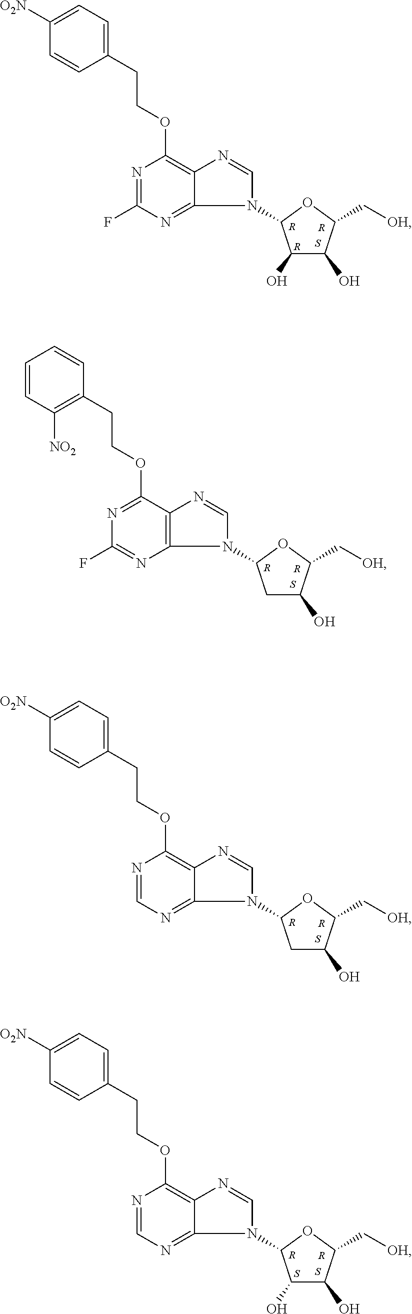

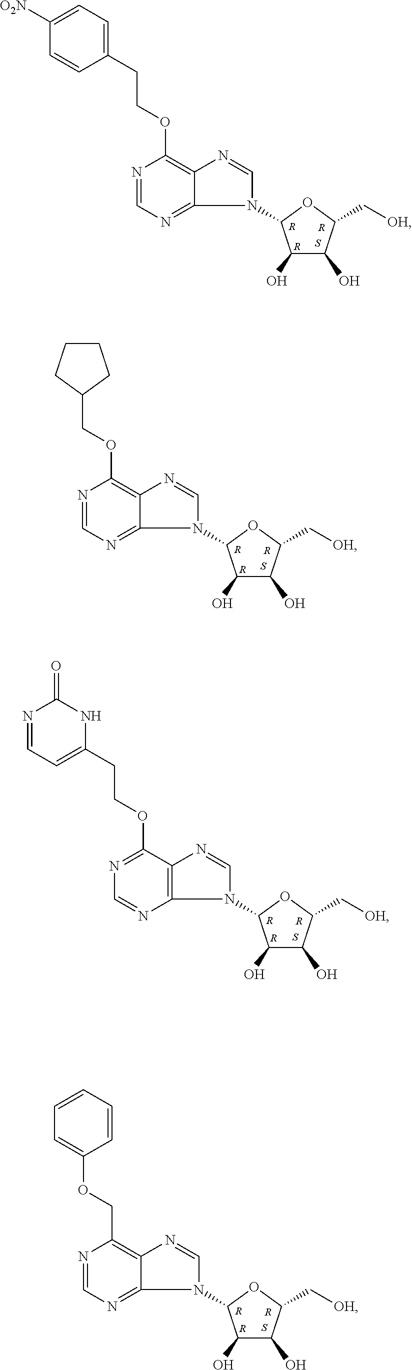

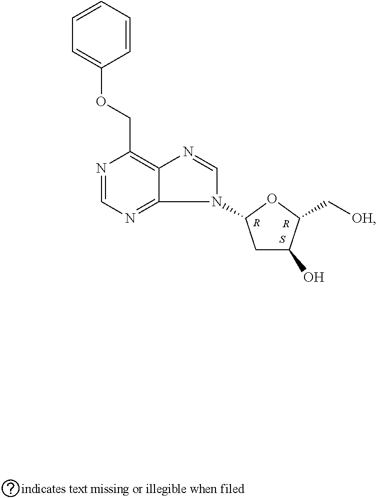

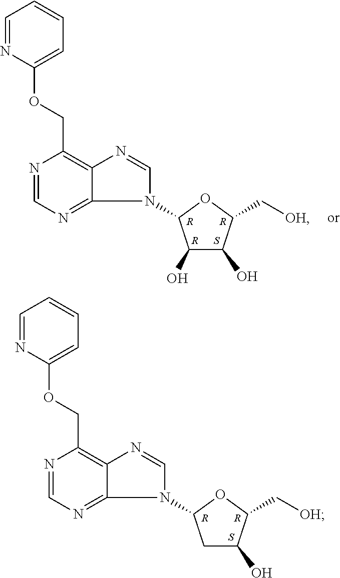

further on the condition that:
(b) when U is chosen from (iv) -alkyl- and (v) -alkenyl-, then:
Ring A is an optionally substituted aryl; and
at least one of Y¹, Y² or Y³ is —OH; and
Z² is -alkyl;
further on the condition that:
(c) when U is chosen from (ix) -alkyl-NR¹— wherein alkyl is attached to Ring A, (x) —NR¹—, (xiii) —CO—, (xi) —C(O)NR¹— wherein the N is attached to Ring A, and (xii) —C(O)NR¹-alkyl- wherein the alkyl is attached to Ring A; then:
Ring A an optionally substituted aryl; and
M is —O— or —CH₂—; and
Z² is -alkyl; and
each R¹ is independently chosen from —H and -alkyl; and
with the proviso that the compound is not

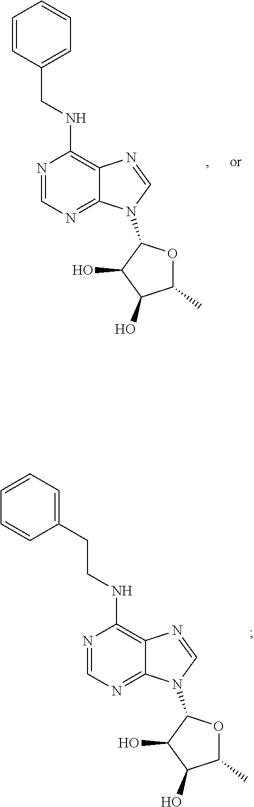 [SMILES: C[C@H]1O[C@@H](n2cnc3c(NCc4ccccc4)ncnc32)[C@H](O)[C@@H]1O ; C[C@H]1O[C@@H](n2cnc3c(NCCc4ccccc4)ncnc32)[C@H](O)[C@@H]1O]

further on the condition that:
(d) when U is chosen from: (vi) -alkyl-S— wherein alkyl is attached to Ring A,
(vii) —SO₂NR¹— wherein the N is attached to Ring A, and (viii) -alkyl-SO₂—NR¹— wherein the N is attached to Ring A; then:
Ring A is C₆ aryl substituted with 1 or 2 groups independently chosen from —NO₂, —CN, —CH₂CN, alkoxy optionally substituted with 1, 2, or 3-halo atoms, and 5-membered heteroaryl optionally substituted with 1, 2, or 3 —CH₃ groups; and
T is —H; and
with the proviso that the compound is not

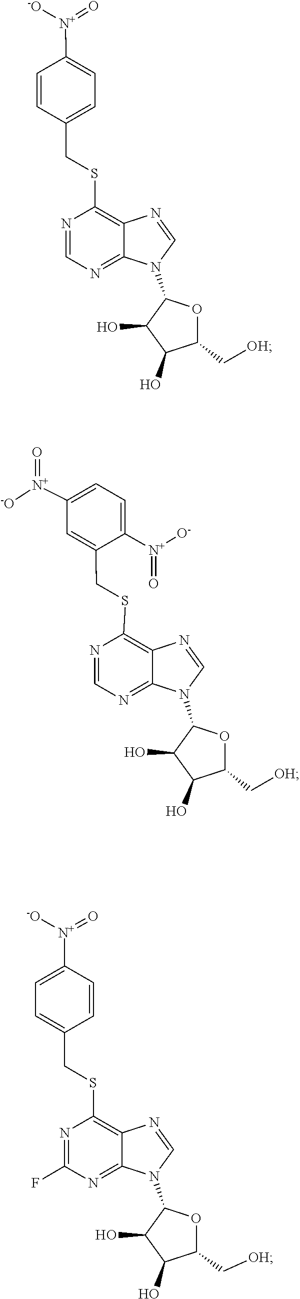

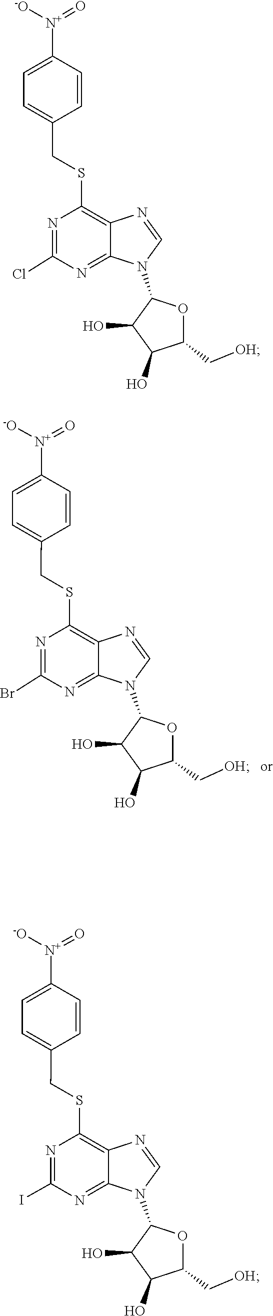

and
further on the condition that:
(e) when U is a direct bond, then
M is —O— or —CH₂—; and
Z² is -alkyl; and
R¹ is —H or -alkyl.

Embodiment 2. A compound of Formula (Ia):

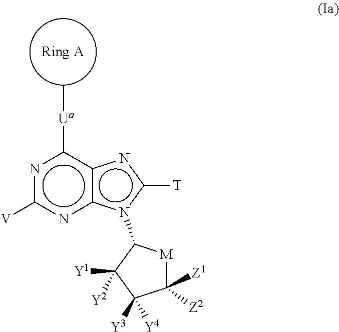

or a pharmaceutically acceptable salt, hydrate, or solvate thereof, wherein:
Ring A is chosen from optionally substituted aryl, optionally substituted heteroaryl containing at least one N, O, or S atom, optionally substituted heterocyclyl containing at least one N or O, and optionally substituted cycloalkyl;
U^{a} is chosen from: (i) —O—, (ii) -alkoxy-, and (iii) -(alkyl)O(alkyl)-;
T is chosen from —H, —OH, —O(alkyl)(aryl), optionally substituted heteroaryl that contains at least one N atom, —C(O)NR¹(cycloalkyl), and optionally substituted amine;
V is chosen from —H, -halo, —OH, -alkyl, and -alkoxy;
M is —O— or —C(R²)₂—;
Y¹ and Y² are each independently chosen from —H, —OH, and -halo;
wherein at least one of Y¹, Y² or Y³ must be —OH;
Y³ is —OH or —H;
Y⁴ is chosen from —H, -halo, —OH, and alkyl optionally substituted with 1, 2, or 3 groups independently chosen from -halo and —OH;
Z¹ is chosen from —H, -halo, —OH, and alkyl optionally substituted with 1, 2, or 3 groups independently chosen from —OH and -halo;
Z² is chosen from —H, -alkyl, -alkenyl, —C(O)NHR¹, —C(O)NR¹(alkyl), and —C(O)O(alkyl), wherein each alkyl and alkenyl group is optionally substituted;
each R¹ is independently chosen from —H and -alkyl; and
each R² is independently chosen from —H, -alkyl, and -cycloalkyl;
wherein when Z² is alkyl, it is not substituted with a phosphonate group or a protected alcohol group; and
with the proviso that the compound is not

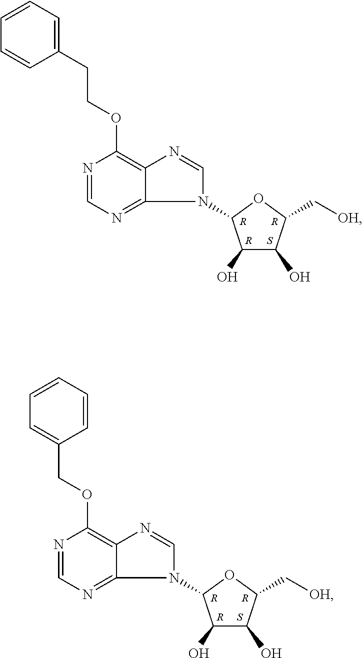

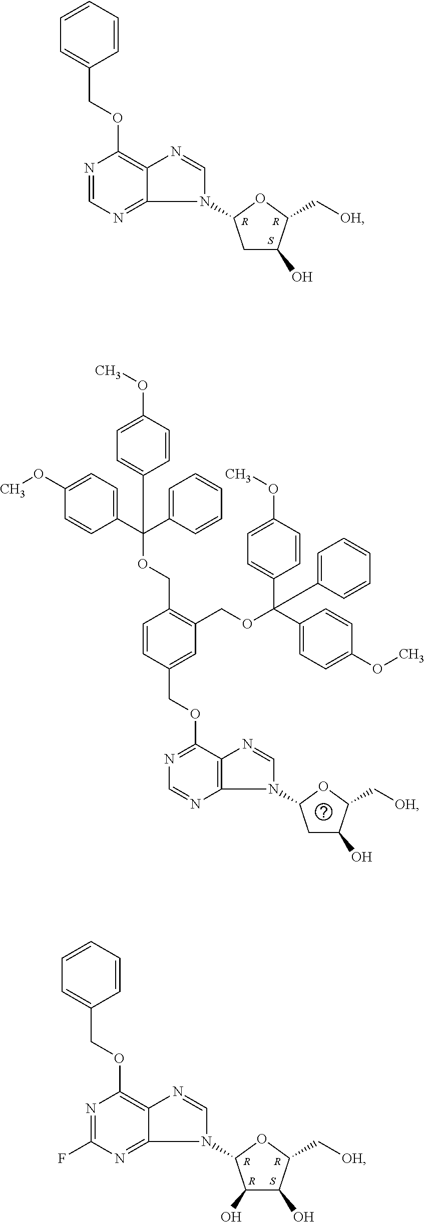

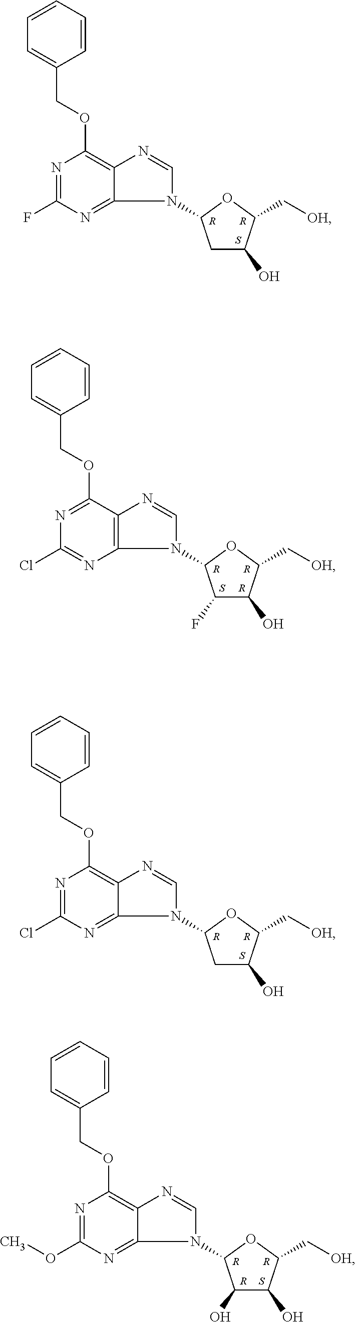

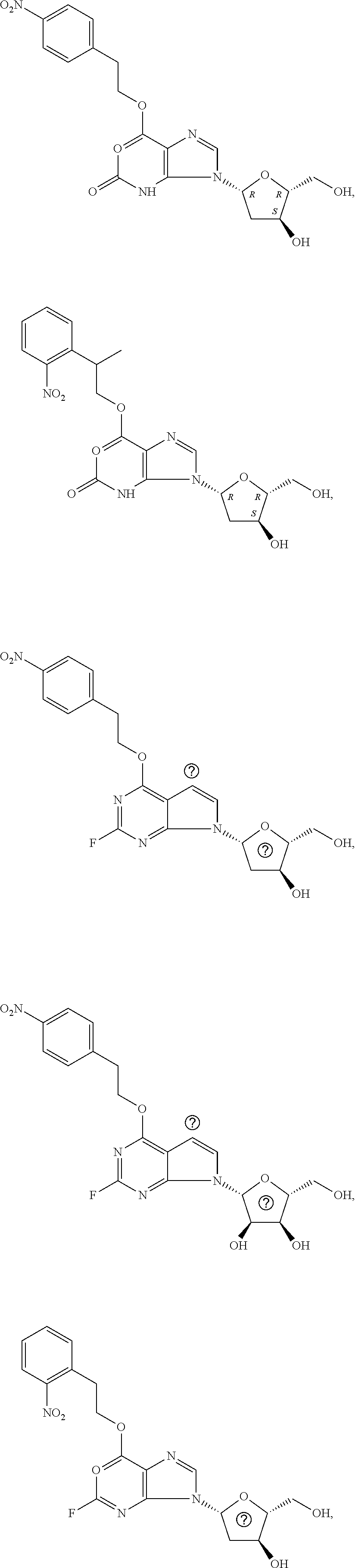

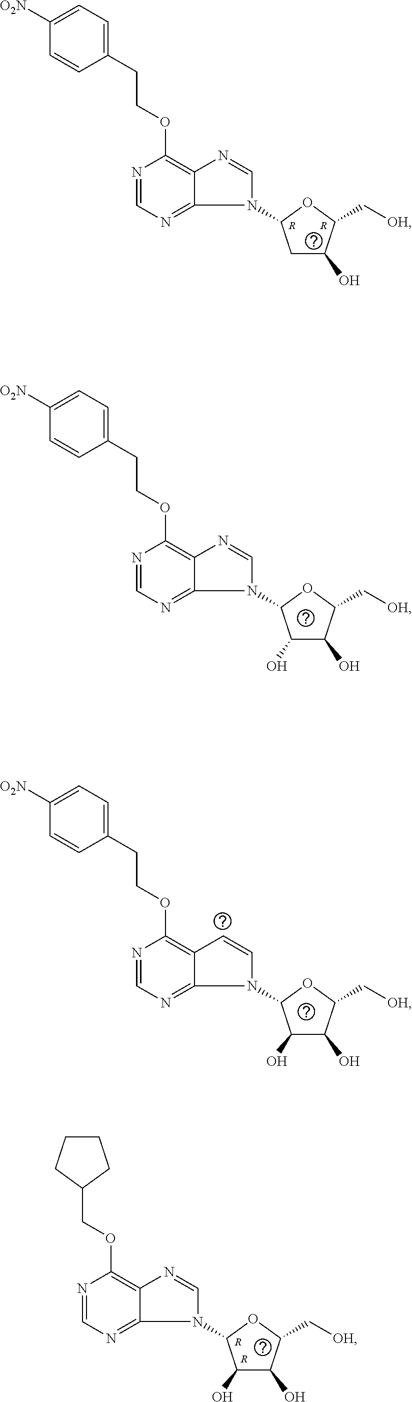

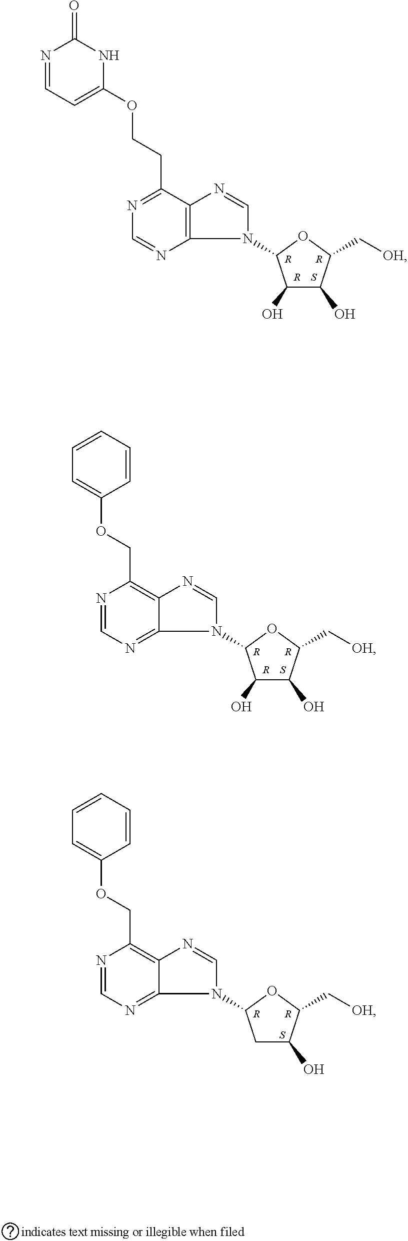

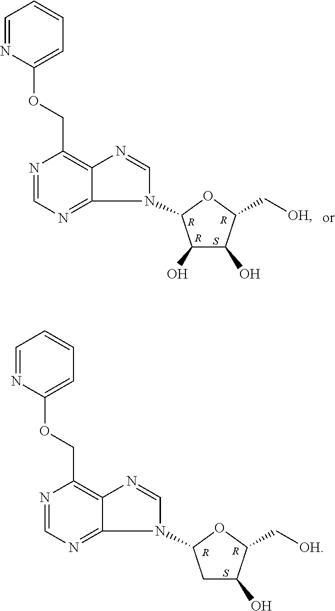

Embodiment 3. The compound according to Embodiment 2, wherein U^{a} is -alkoxy- wherein the alkyl group of the alkoxy group is attached to Ring A.

Embodiment 4. The compound according to Embodiment 2, wherein U^{a} is —CH₂O— wherein the —CH₂— is attached to Ring A.

Embodiment 5. The compound according to Embodiments 2-4, wherein Z² is -alkyl optionally substituted with 1 or 2 groups independently chosen from —OCH₂CH₂OH, —OH, —OCH₂C(O)NH₂ and —C(O)OCH₂CH₃, or Z² is -alkenyl substituted with —C(O)CH₂CH₃.

Embodiment 6. The compound according to any one of Embodiments 2-5, wherein Z² is —CH₃.

Embodiment 7. The compound according to any one of Embodiments 2-6, wherein Ring A is chosen from an optionally substituted C₆-aryl and an optionally substituted 6 membered heteroaryl.

Embodiment 8. The compound according to any one of Embodiments 2-7, wherein Ring A is chosen from phenyl, pyridinyl, and pyridazinyl, each of which is optionally substituted.

Embodiment 9. The compound according to any one of Embodiments 2-8, wherein Ring A is phenyl optionally substituted with 1, 2, or 3 groups independently chosen from —NO₂, —CH₃, —OCHF₂, —OCF₃, —OCH₂CF₃, —CN, —F, —Cl, —CHF₂, —CF₃, and -cyclopropyl.

Embodiment 10. The compound according to any one of Embodiments 2-8, wherein Ring A is pyridinyl optionally substituted with 1, 2, or 3 groups independently chosen from —NO₂, —CH₃, —OCHF₂, —OCF₃, —OCH₂CF₃, —CN, —F, —Cl, —CHF₂, —CF₃, and -cyclopropyl.

Embodiment 11. The compound according to any one of Embodiments 2-10, wherein T is chosen from —H, —O(CH₂)(C₆ aryl), —C(O)NR¹(C₃₋₅ cycloalkyl), optionally substituted 5 membered heteroaryl, and an optionally substituted amine.

Embodiment 12. The compound according to any one of Embodiments 2-11, wherein T is an optionally substituted amine chosen from —N(R¹)₂, —NR¹(C₁₋₄ alkyl), —NR¹(C₃₋₆ cycloalkyl), —NR¹(CH₂)₁₋₂(C₃₋₆ cycloalkyl), —NR¹(5-6 membered heterocyclyl), —NR¹(CH₂)₁₋₂(5-6 membered heterocyclyl), —NR₁(5-6 membered heteroaryl), —NR¹(CH₂)₁₋₂(5-6 membered heteroaryl), —NR¹(C₅₋₆ aryl) and —NR¹(CH₂)₁₋₂(C₅₋₆ aryl), wherein each alkyl, cycloalkyl, heterocyclyl, heteroaryl, or aryl is optionally substituted with 1, 2, or 3 groups independently chosen from —OH, -halo, —CN, —CH₃, —C(O)CH₃, —OCH₃, —SO₂CH₃, and —O—(C₆ aryl).

Embodiment 13. A compound of Formula (Ib):

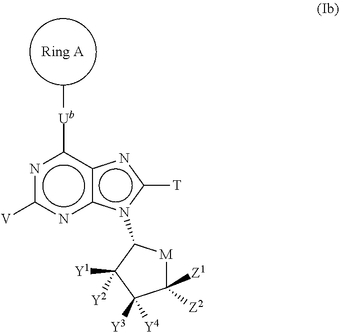

or a pharmaceutically acceptable salt, hydrate, or solvate thereof, wherein:
Ring A is an optionally substituted aryl;
U^{b} is: (i) -alkyl- or (ii) -alkenyl-;
T is chosen from —H, —OH, —O(alkyl)(aryl), optionally substituted heteroaryl that contains at least one N atom, —C(O)NR¹(cycloalkyl), and optionally substituted amine;
V is chosen from —H, -halo, —OH, -alkyl, and -alkoxy;
M is —O— or —C(R²)₂—;
Y¹ and Y² are each independently chosen from —H, —OH and -halo;
   wherein at least one of Y¹, Y² or Y³ must be —OH;
Y³ is —OH or —H;
Y⁴ is chosen from —H, -halo, —OH, and -alkyl optionally substituted with 1, 2, or 3 groups independently chosen from -halo and —OH;
Z¹ is chosen from —H, -halo, —OH, and -alkyl optionally substituted with 1, 2, or 3 groups independently chosen from —OH and -halo;
Z² is -alkyl;
R¹ is chosen from —H and -alkyl; and
each R² is independently chosen from —H, -alkyl, and -cycloalkyl.

Embodiment 14. The compound according to Embodiment 13, wherein U^{b} is —CH₂—, —CH₂CH₂— or —CH═CH—.

Embodiment 15. The compound according to Embodiment 13 or 14, wherein Z² is —CH₃.

Embodiment 16. The compound according to any one of Embodiments 13-15, wherein Ring A is C₆ aryl optionally substituted with 1, 2, or 3 groups independently chosen from —NO₂, —CH₃, —OCHF₂, —OCF₃, —OCH₂CF₃, —CN, —F, —Cl, —CHF₂, —CF₃, and -cyclopropyl.

Embodiment 17. The compound according to any one of Embodiments 13-16, wherein T is chosen from —H, —O(CH₂)(C₆ aryl), —C(O)NR¹(C₃₋₅ cycloalkyl), optionally substituted 5 membered heteroaryl that contains at least one N atom, and an optionally substituted amine.

Embodiment 18. The compound according to any one of Embodiments 13-17, wherein T is —H.

Embodiment 19. A compound of Formula (Ic):

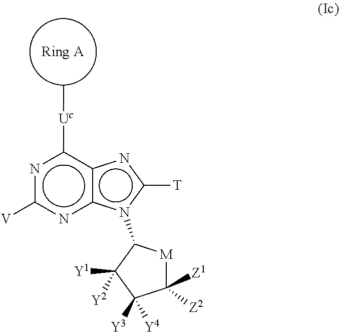

or a pharmaceutically acceptable salt, hydrate, or solvate thereof, wherein:
Ring A is optionally substituted aryl;
U^{c} is chosen from: (i) -alkyl-NR¹— wherein alkyl is attached to Ring A, (ii) —NR—,
(iii) —CO—, (iv) —C(O)NR¹— wherein the N is attached to Ring A, and (v) —C(O)NR¹-alkyl- wherein the alkyl is attached to Ring A;
T is chosen from —H, —OH, —O(alkyl)(aryl), optionally substituted heteroaryl that contains at least one N atom, —C(O)NR¹(cycloalkyl), and optionally substituted amine;
V is chosen from —H, -halo, —OH, -alkyl, and -alkoxy;
M is —O— or —C(R²)₂—;
Y³ is —OH or —H;
Y¹ and Y² are each —H, —OH or -halo;
Y⁴ is chosen from —H, -halo, —OH, and -alkyl optionally substituted with 1, 2, or 3 groups independently chosen from -halo and —OH;
Z¹ is chosen from —H, -halo, —OH, and -alkyl optionally substituted with 1, 2, or 3 groups independently chosen from —OH and -halo;
Z₂ is -alkyl; and
each R¹ is independently chosen from —H and -alkyl; and
each R² is independently chosen from —H, -alkyl, and -cycloalkyl;
with the proviso that the compound is not

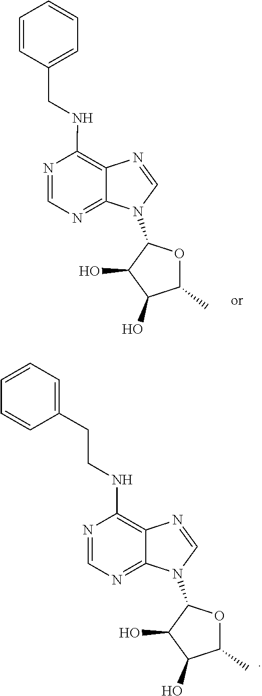 [SMILES: C[C@H]1O[C@@H](n2cnc3c(NCc4ccccc4)ncnc32)[C@H](O)[C@@H]1O ; C[C@H]1O[C@@H](n2cnc3c(NCCc4ccccc4)ncnc32)[C@H](O)[C@@H]1O]

Embodiment 20. The compound according to Embodiment 19, wherein U^{c} is chosen from -alkyl-NR¹— wherein alkyl is attached to Ring A, and —NR¹—.

Embodiment 21. The compound according to Embodiment 19 or 20, wherein U° is —(CH₂)₁₋₂—NR¹— wherein —(CH₂)₁₋₂— is attached to Ring A.

Embodiment 22. The compound according to any one of Embodiments 19-21, wherein Z² is —CH₃.

Embodiment 23. The compound according to any one of Embodiments 19-22, wherein Ring A is C₆-aryl optionally substituted with 1, 2 or 3 groups independently chosen from —NO₂, —CH₃, —OCHF₂, —OCF₃, —OCH₂CF₃, —CN, —F, —Cl, —CHF₂, —CF₃, and -cyclopropyl.

Embodiment 24. The compound according to any one of Embodiments 19-23, wherein T is chosen from —H, —O(CH₂)(C₆ aryl), —C(O)NR₁(C₃₋₅ cycloalkyl), optionally substituted 5 membered heteroaryl that contains at least one N atom, and an optionally substituted amine.

Embodiment 25. The compound according to any one of Embodiments 19-24, wherein T is —H.

Embodiment 26. A compound of Formula (Id):

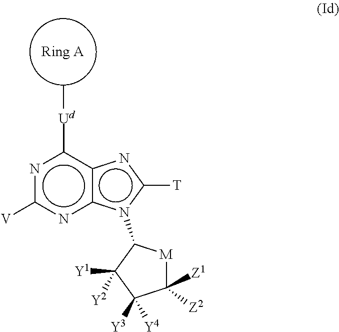

or a pharmaceutically acceptable salt, hydrate, or solvate thereof, wherein:
Ring A is C₆ aryl substituted with 1 or 2 groups independently chosen from —NO₂, —CN, —CH₂CN, -alkoxy optionally substituted with 1, 2, or 3-halo atoms, and 5 membered heteroaryl optionally substituted with 1 or 2 —CH₃ groups;
U^{d} is chosen from: (i) -alkyl-S— wherein alkyl is attached to Ring A, (ii) —SO₂NR¹— wherein the N is attached to Ring A, and (iii) -alkyl-SO₂—NR¹— wherein the N is attached to Ring A;
T is —H;
V is chosen from —H, -halo, —OH, -alkyl, and -alkoxy;
M is —O— or —C(R²)₂—;
Y¹ and Y² are each independently chosen from —H, —OH and -halo;
Y³ is —OH or —H;
Y⁴ is chosen from —H, -halo, —OH, and -alkyl optionally substituted with 1, 2, or 3 groups independently chosen from -halo and —OH;
Z¹ is chosen from —H, -halo, —OH, and -alkyl optionally substituted with 1, 2, or 3 groups independently chosen from —OH and -halo;
Z² is chosen from —H, -alkyl, -alkenyl, —C(O)NHR¹, —C(O)NR¹(alkyl), and —C(O)O(alkyl), wherein each alkyl and alkenyl group is optionally substituted;
each R¹ is independently chosen from —H and -alkyl; and
each R² is independently chosen from —H, -alkyl, and -cycloalkyl;
with the proviso that the compound is not

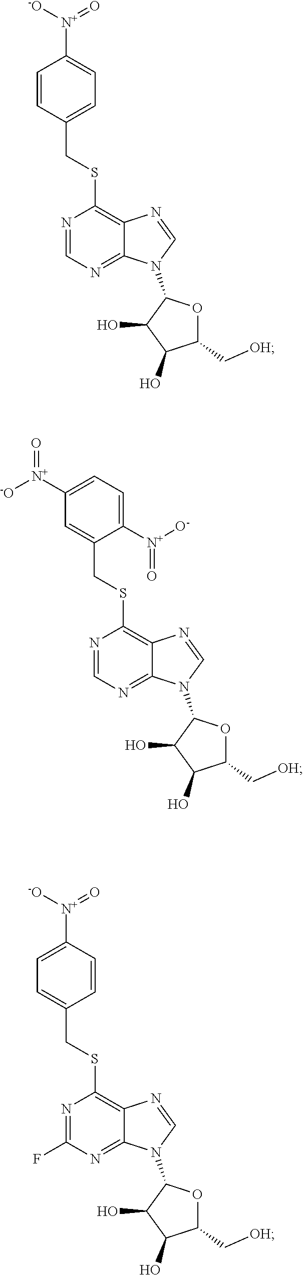

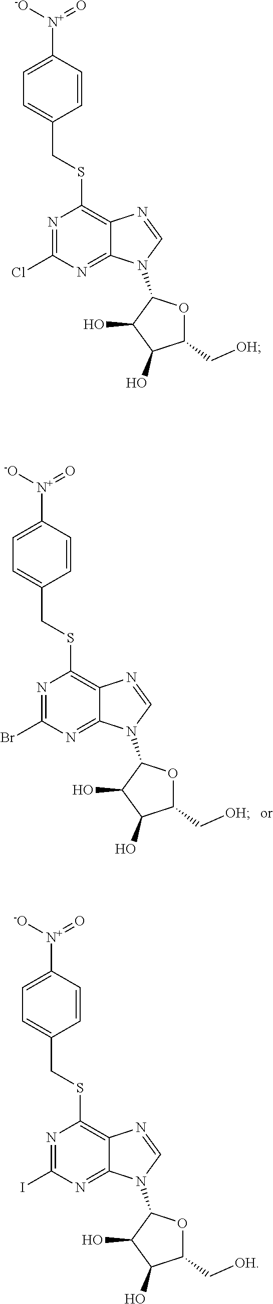

Embodiment 27. The compound according to Embodiment 26, wherein U^{d} is -alkyl-S— wherein alkyl is attached to Ring A.

Embodiment 28. The compound according to Embodiment 26 or 27, wherein U^{d} is —(CH₂)₁₋₂—S— wherein —(CH₂)₁₋₂— is attached to Ring A.

Embodiment 29. The compound according to any one of Embodiments 26-28, wherein Z² is chosen from optionally substituted -alkyl and —C(O)OCH₃.

Embodiment 30. The compound according to any one of Embodiments 26-29, wherein Z² is an optionally substituted -alkyl, wherein the alkyl is not substituted with a phosphonate group or a protected alcohol group.

Embodiment 31. The compound according to any one of Embodiments 26-30, wherein Z² is chosen from —CH₃, —CH₂OH, —CH₂F, and —CH₂NHC(O)CH₃.

Embodiment 32. The compound according to any one of Embodiments 26-31, wherein Ring A is an optionally substituted C₆ aryl optionally substituted with 1 or 2 groups independently chosen from —NO₂, —CN, —CH₂CN, -alkoxy optionally substituted with 1, 2, or 3 —F atoms, and 5 membered heteroaryl optionally substituted with 1 —CH₃ group.

Embodiment 33. The compound according to any one of Embodiments 26-32, wherein Ring A is a C₆ aryl optionally substituted with 1 or 2 groups independently chosen from —NO₂, —CN, —CH₂CN, —OCHF₂, —OCH₂CF₃ and 1,2,4-oxadiazole optionally substituted with 1 —CH₃ group.

Embodiment 34. A compound of Formula (Ie):

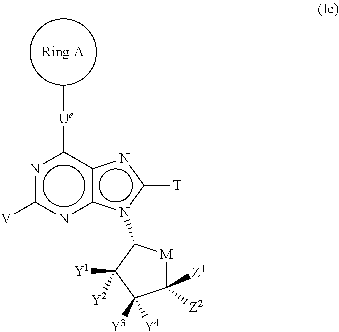

or a pharmaceutically acceptable salt, hydrate, or solvate thereof, wherein:
Ring A is chosen from optionally substituted aryl, optionally substituted heteroaryl containing at least one N, O, or S atom, optionally substituted heterocyclyl containing at least one N or O, and optionally substituted cycloalkyl;
U^{e} is a direct bond;
T is chosen from —H, —OH, —O(alkyl)(aryl), optionally substituted heteroaryl that contains at least one N atom, —C(O)NR¹(cycloalkyl), and optionally substituted amine;
V is chosen from —H, -halo, —OH, -alkyl, and -alkoxy;
M is —O— or —C(R²)₂—;
Y¹ and Y² are each independently chosen from —H, —OH, and -halo;
Y³ is —OH or —H;
Y⁴ is chosen from —H, -halo, —OH, and -alkyl optionally substituted with 1, 2, or 3 groups independently chosen from -halo and —OH;
Z¹ is chosen from —H, -halo, —OH, and -alkyl optionally substituted with 1, 2, or 3 groups independently chosen from —OH and -halo;
Z² is -alkyl;
R¹ is —H or -alkyl; and
each R² is independently chosen from —H, -alkyl, and -cycloalkyl.

Embodiment 35. The compound according to Embodiment 34, wherein Z² is —CH₃.

Embodiment 36. The compound according to Embodiment 34 or 35, wherein Ring A is chosen from an optionally substituted C₆-10 cycloalkyl, an optionally substituted 4-10 membered heteroaryl containing at least one N, O, or S atom, and an optionally substituted 4-6 membered heterocyclyl containing at least one N or O atom.

Embodiment 37. The compound according to any one of Embodiments 34-36, wherein Ring A is chosen from

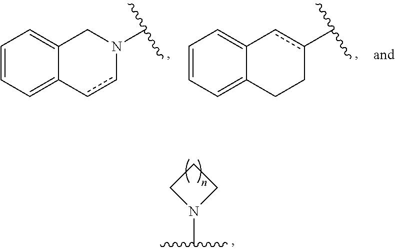

each of which is optionally substituted, wherein the [symbol] is a single or double bond and wherein n is 1, 2 or 3.

Embodiment 38. The compound according to any one of Embodiments 34-37, wherein Ring A is optionally substituted with 1, 2, or 3 groups independently chosen from —NO₂, —CH₃, —OCHF₂, —OCF₃, —OCH₂CF₃, —CN, —F, —Cl, —CHF₂, —CF₃, and -aryl optionally substituted with —NO₂.

Embodiment 39. The compound according to any one of Embodiments 34-38, wherein T is —H.

Embodiment 40. The compound according to any one of Embodiments 34-38, wherein T is an optionally substituted amine chosen from —NH₂, —NR¹(C₁₋₄ alkyl), —NR¹(C₃₋₆ cycloalkyl), —NR¹CH₂(C₃₋₆ cycloalkyl), —NR¹(5-6 membered heterocyclyl), —NR¹CH₂(5-6 membered heterocyclyl), —NR¹(5-6 membered heteroaryl), —NR¹CH₂(5-6 membered heteroaryl), —NR¹(C₅₋₆ aryl), and —NR¹CH₂(C₅₋₆ aryl), wherein each alkyl, cycloalkyl, heterocyclyl, heteroaryl, or aryl is optionally substituted with 1, 2, or 3 groups independently chosen from —OH, -halo, —CN, —CH₃, —C(O)CH₃, —OCH₃, —S(O)₂CH₃, and —O-phenyl.

Embodiment 41. The compound according to any of the preceding Embodiments, wherein M is —O— or —CH₂—.

Embodiment 42. The compound according to any of the preceding Embodiments, wherein M is —O—.

Embodiment 43. The compound according to any of the preceding Embodiments, wherein V is —H or -halo.

Embodiment 44. The compound according to any of the preceding Embodiments, wherein V is —F.

Embodiment 45. The compound according to any of Embodiments 1-43, wherein V is —H.

Embodiment 46. The compound according to any of the preceding Embodiments, wherein Y² and Y⁴ are both —H.

Embodiment 47. The compound according to any of the preceding Embodiments, wherein Y¹ and Y³ are both —OH.

Embodiment 48. The compound according to any of the preceding Embodiments, wherein Z¹ is —H.

Embodiment 49. The compound according to any of the preceding Embodiments, wherein each R¹ is —H.

Embodiment 50. The compound according to any one of Embodiments 1-48, wherein each R¹ is —CH₃.

Embodiment 51. The compound according to any of the preceding Embodiments, wherein each R² is —H.

Embodiment 52. The compound according to any one of Embodiments 1-50, wherein one R² is —H and one R² is —CH₃.

Embodiment 53. A compound chosen from the compounds listed in Table 1, or a pharmaceutically acceptable salt, hydrate, or solvate thereof.

Embodiment 54. A pharmaceutical composition comprising at least one compound according to any one of Embodiments 1-53, or a pharmaceutically acceptable salt, hydrate, or solvate thereof, and a pharmaceutically acceptable excipient.

Embodiment 55. A method of inhibiting ENT1 in a patient need thereof, comprising administering to the patient an effective amount of at least one compound according to any one of Embodiments 1-53.

Embodiment 56. A method of treating cancer in a patient need thereof, comprising administering to the patient an effective amount of at least one compound according to any one of Embodiments 1-53.

Embodiment 57. A method of treating cancer in a patient need thereof, comprising administering to the patient at least one compound chosen from those disclosed herein and an adenosine receptor antagonist.

Embodiment 58. The method according to Embodiments 57, wherein the adenosine receptor antagonist is (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one, or a pharmaceutically acceptable salt, hydrate, or solvate thereof.

Embodiment 59. A kit of parts comprising: (a) a first part comprising an effective amount of at least one compound according to any one of Embodiments 1-53; and (b) a second part comprising an effective amount of an adenosine receptor antagonist.

Embodiment 60. The kit according to Embodiments 59, wherein the adenosine receptor antagonist is (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one, or a pharmaceutically acceptable salt, hydrate, or solvate thereof.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl). Solvates or hydrates of the compounds described herein can be prepared by standard methods.

The following abbreviations are used:
ACN or MeCN: acetonitrile;
AcOH: acetic acid;
AIBN: azobisisobutyronitrile;
BH₃·Me₂S: borane dimethyl sulfide complex;
BH₃·THF: borane tetrahydrofuran complex;
brine: saturated aqueous solution of sodium chloride;
BSA: bis(trimethylsilyl)acetamide;
CDCl₃: deuterated chloroform;
Cs₂CO₃: cesium carbonate;
DABCO: 1,4-diazabicyclo[2.2.2]octane;
DBTCE: 1,2-dibromotetrachloroethane;
DCM: dichloromethane;
DEAD: diethyl azodicarboxylate;
DIAD: diisopropyl azodicarboxylate
DIBAl-H: diisobutylaluminium hydride;
DIEA: N,N-diisopropylethylamine;
DMF: dimethylformamide;
DMSO: dimethyl sulfoxide;
EA: ethyl acetate;
e.e.: enantiomeric excess;
EtOAC: ethyl acetate;
eq: equivalence;
FA: formic acid;
HPLC: high pressure liquid chromatography;
hr: hour(s);
IBX: 2-iodoxybenzoic acid;
K2CO₃: potassium carbonate;
LDA: lithium diisopropylamide;
LiBH₄: lithium borohydride;
MeOH: methyl alcohol;
min: minute;
MsCl: methanesulfonyl chloride;
N₂: nitrogen gas;
Na₂CO₃: sodium carbonate;
Na₂HPO₄: disodium hydrogen phosphate;
Na₂SO₄: sodium sulfate;
NaBH₄: sodium borohydride;
NaHCO₃: sodium bicarbonate;
NBS: N-Bromosuccinimide;
NH₃·H2O: ammonium hydroxide solution;
NH₄F: ammonium fluoride;
Pd₂(dba)₃: tris(dibenzylideneacetone)dipalladium(0);
Pd₂(dppf)Cl₂·DCM: [1,1′-Bis(diphenylphosphino)ferrocene]dichloropalladium(II), complex with dichloromethane;
PE: petroleum ether;
PPh₃: triphenylphosphine;
Prep-HPLC: preparative high pressure liquid chromatography;
Prep-TLC: preparative thin layer chromatography;
Rf: retention factor;
Rt: retention time;
sat.: saturated;
SFC: supercritical fluid chromatography;
SiO₂: silica gel;
Sphos: dicyclohexyl(2′,6′-dimethoxy[1,1′-biphenyl]-2-yl)phosphane
T₃P: propanephosphonic acid anhydride;
TBSCl: tert-butyldimethylsilyl chloride;
TEA: triethylamine;
TEMPO: (2,2,6,6-tetramethylpiperidin-1-yl)oxyl
TFA: trifluoroacetic acid;
THF: tetrahydrofuran;
TLC: thin layer chromatography;
TMSOTf: trimethylsilyl trifluoromethanesulfonate;
TsOH: p-toluenesulfonic acid;
TsOH·H2O: p-toluenesulfonic acid monohydrate;

The crossed double bond (shown as [symbol] is used to indicate that a double bond may be cis or trans. Compounds shown below with the crossed double bond indicates that the isolated compound may be completely cis, completely trans, or a mixture of cis and trans.

### I. Chemistry Examples

The MS data provided in the examples described below were obtained as follows: LCMS were recorded using Agilent 6130 or 6130B multimode (ESI+APCI).

### LCMS Methods

### Method A

This method was used for the LCMS analysis of intermediate compounds. The column used for chromatography was a ZORBAX Eclipse XDB-C18 2.1×30 mm (3.5 m particles). Detection method was diode array (DAD). MS mode was positive electrospray ionization. MS range was 100-1000 m/z. Mobile phase A was 0.037% trifluoroacetic acid in water, and mobile phase B was 0.018% trifluoroacetic acid in HPLC grade acetonitrile. The gradient was 5-95% B in 2.20 min, 5% B in 0.01 min, 5-95% B (0.01-1.00 min), 95-100% B (1.00-1.80 min), 5% B in 1.81 min with a hold at 5% B for 0.39 min. The flow rate was 1.0 mL/min.

### Method B

This method was used for the LCMS analysis of final compounds. The column used for chromatography was a Kinetex C18 50×2.1 mm column (5 m particles). Detection methods were diode array (DAD) and evaporative light scattering detection (ELSD) as well as positive electrospray ionization. MS range was 100-1000. Mobile phase A was 0.037% trifluoroacetic acid in water, and mobile phase B was 0.018% trifluoroacetic acid in acetonitrile. The gradient was 5% B in 0.40 min and 5-95% B at 0.40-3.00 min, hold on 95% B for 1.00 min, and then 95-5% B in 0.01 min, the flow rate was 1.0 ml/min.

### Reverse Phase HPLC Purification

Reverse phase IPLC was used for the analysis of compounds. A Gilson GX-281 instrument was used with a Flash Spherical C18 (Spherical; 20-35 μm; 100A) column. The flow rate was 60 mL/min, column temperature was room temperature, and the detection wavelength was 220 nm/254 nm. The following mobile phases were used.
Basic conditions:NH₃·H2O: Mobile phase: A for H₂O (0.1% NH₃·H₂O v/v) and B for Acetonitrile

### Acidic Conditions:

TFA: Mobile phase: A for H₂O (0.1% TFA v/v) and B for Acetonitrile
FA: Mobile phase: A for H₂O (0.1% FA v/v) and B for Acetonitrile
HCl: Mobile phase: A for H₂O (0.1% HC v/v) and B for Acetonitrile
   Neutral conditions:
H₂O. Mobile phase: A for H₂O and B for Acetonitrile,
NH₃·HCO₃: Mobile phase: A for H₂O (0.1%. NH₂HCO₃) and B for Acetonitrile

### NMR Analysis

The NMR data provided in the examples described below were obtained as followed:

1H-NMR: Bruker DPX 400 MHz. Abbreviations for multiplicities observed in NMR spectra are as follows: s (singlet), d (doublet), ddd (doublet of doublet of doublets), dt (doublet of triplets), dq (double of quartets), t (triplet), td (triplet of doublets), tt (triplet of triplets), q (quadruplet), m (multiplet), br (broad singlet).

Solvents, reagents and starting materials were purchased and used as received from commercial vendors unless otherwise specified.

### Synthesis of Starting Materials

### (6-(difluoromethoxy)pyridin-3 yl)methanol

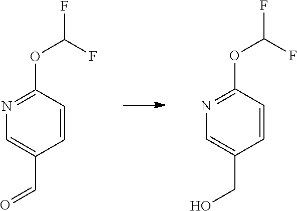 [SMILES: O=Cc1ccc(OC(F)F)nc1 ; OCc1ccc(OC(F)F)nc1]

To a solution of 6-(difluoromethoxy)nicotinaldehyde (50 mg, 288.82 μmol, 1 eq) in MeOH (1.5 mL) was added NaBH₄ (13.11 mg, 346.59 μmol, 1.2 eq) at 0° C. The mixture was stirred at 0° C. for 1 hr. The mixture was then poured into a cold solution of 1 N HCl (10 mL). The aqueous phase was extracted with ethyl acetate (5 mL×2). The combined organic phase was washed with brine (10 mL), dried with anhydrous Na₂SO₄, filtered and concentrated under vacuum to afford (6-(difluoromethoxy)pyridin-3-yl)methanol (50 mg, 99% yield) as a colorless oil.

1H NMR (400 MHz, CDCl₃-d) δ=8.18 (d, J=1.8 Hz, 1H), 7.81-7.65 (m, 1H), 7.49-7.28 (m, 1H), 6.92 (d, J=8.4 Hz, 1H), 4.71 (s, 2H).

### (6-nitropyridin-3-yl)methanol

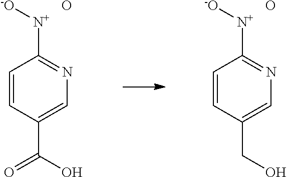

To a solution of 6-nitronicotinic acid (240 mg, 1.43 mmol, 1 eq) in THE (3 mL) was added BH3·THF (1 M, 8 mL, 5.6 eq) dropwise at 0° C. The mixture was warmed to 20° C. for 5 hr under N₂ atmosphere. The reaction mixture was quenched by MeOH at 0° C. under N₂ atmosphere. Then the mixture was stirred at 80° C. for 1 hr. The solvent was removed under reduced pressure to give a residue, which was purified by Prep-TLC (SiO₂, PE/EtOAc=0/1) to afford (6-nitropyridin-3-yl)methanol (170 mg, 77% yield) as a white solid.

1H NMR (400 MHz, DMSO-d6) δ=8.59 (s, 1H), 8.30 (d, J=8.4 Hz, 1H), 8.18-8.09 (m, 1H), 5.64 (t, J=5.6 Hz, 1H), 4.70 (d, J=5.5 Hz, 2H).

### (6-(trifluoromethoxy)pyridin-3-yl)methanol

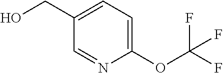 [SMILES: OCc1ccc(OC(F)(F)F)nc1]

(6-(trifluoromethoxy)pyridin-3-yl)methanol was prepared following protocols described for (6-(difluoromethoxy)pyridin-3-yl)methanol from the corresponding 6-(trifluoromethoxy)nicotinic acid.

1H NMR (400 MHz, CDCl₃-d) δ=8.31 (d, J=2.2 Hz, 1H), 7.85 (dd, J=2.4, 8.4 Hz, 1H), 7.04 (d, J=8.4 Hz, 1H), 4.60 (s, 2H).

### (6-(difluoromethyl)pyridin-3-yl)methanol

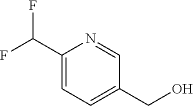 [SMILES: OCc1ccc(C(F)F)nc1]

(6-(difluoromethyl)pyridin-3-yl)methanol was prepared following protocols described for (6-(difluoromethoxy)pyridin-3-yl)methanol from the corresponding 6-(difluoromethyl)nicotinic acid.

1H NMR (400 MHz, CDCl₃-d) δ=7.55-7.44 (m, 4H), 6.83-6.48 (m, 1H), 4.77 (s, 2H).

### 2,6-difluoro-4-(hydroxymethyl)benzonitrile

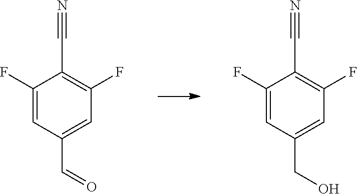 [SMILES: N#Cc1c(F)cc(C=O)cc1F ; N#Cc1c(F)cc(CO)cc1F]

To a solution of 2,6-difluoro-4-formyl-benzonitrile (100 mg, 598.40 μmol, 1 eq) in MeOH (3 mL) was added NaBH₄ (22.64 mg, 598.40 μmol, 1 eq) at 0° C. The mixture was stirred at 0° C. for 1 hr. The mixture was then poured into ice-HCl (1 N 10 mL). The aqueous phase was extracted with ethyl acetate (5 mL×2). The combined organic phase was washed with brine (10 mL), dried with anhydrous Na₂SO₄, filtered and concentrated under vacuum. 2,6-difluoro-4-(hydroxymethyl)benzonitrile (100 mg, 99% yield) was obtained without purification as a yellow solid.

### S-(4-fluoro-3,5-dimethylbenzyl) ethanethioate

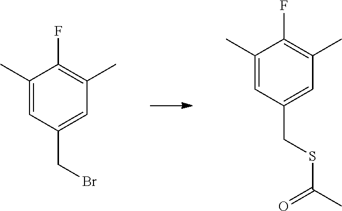

To a mixture of 5-(bromomethyl)-2-fluoro-1,3-dimethylbenzene (200 mg, 921.33 mol, 1 eq) and K2CO₃ (140 mg, 1.01 mmol, 1.1 eq) in acetone (2 mL) was added ethanethioic S-acid (140.26 mg, 1.84 mmol, 131.08 μL, 2 eq). The mixture was stirred at 15° C. for 12 hours. The mixture was poured into ice-water (5 mL). The aqueous phase was extracted with ethyl acetate (5 mL×2). The combined organic phase was washed with brine (5 mL), dried with anhydrous Na₂SO₄, filtered and concentrated under vacuum to afford S-(4-fluoro-3,5-dimethylbenzyl) ethanethioate (200 mg, crude) as yellow oil.

LCMS: Rt=0.993 min; (ESI positive ion) m/z: 235.1 (M+Na)+(calculated: 235.06).

Other S-alkyl ethanethioates were prepared in a similar manner using the same molar equivalents.

### 4,4,5,5-tetramethyl-2-(4-nitrostyryl)-1,3,2-dioxaborolane

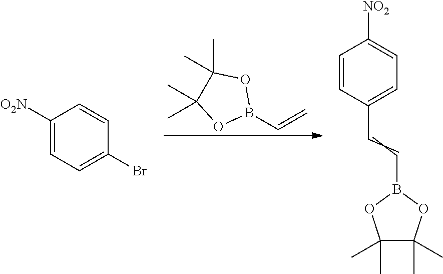

A mixture of 1-bromo-4-nitrobenzene (1.3 g, 14.85 mmol, 1 eq), 4,4,5,5-tetramethyl-2-vinyl-1,3,2-dioxaborolane (2, 4.57 g, 29.70 mmol, 5.04 mL, 2 eq), Pd(OAc)₂ (333.42 mg, 1.49 mmol, 0.1 eq), SPhos (1.22 g, 2.97 mmol, 0.2 eq) and TEA (6.01 g, 59.40 mmol, 8.27 mL, 4 eq) in dioxane (40 mL) was degassed and purged with N₂ 3 times. The mixture was stirred at 80° C. for 16 hours under N₂ atmosphere. The reaction mixture was filtered and concentrated under reduced pressure to give a residue, which was purified by column chromatography (SiO₂, Petroleum ether:Ethyl acetate=1:0 to 5:1) to afford 4,4,5,5-tetramethyl-2-(4-nitrostyryl)-1,3,2-dioxaborolane (3, 1 g, 24% yield) as a yellow solid.

### 6-(2-hydroxyethyl)pyridazine-3-carbonitrile

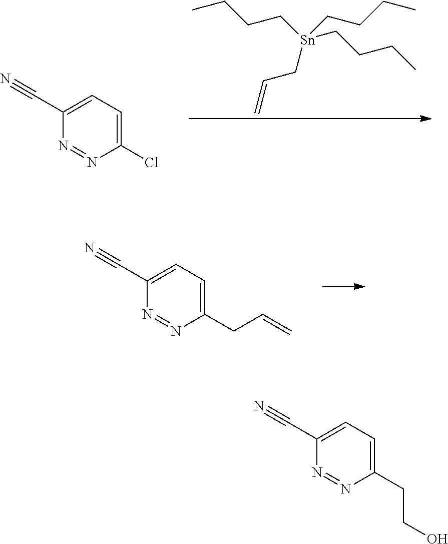 [SMILES: N#Cc1ccc(Cl)nn1 ; C=C[CH2][Sn]([CH2]CCC)([CH2]CCC)[CH2]CCC ; C=CCc1ccc(C#N)nn1 ; N#Cc1ccc(CCO)nn1]

To a mixture of 6-chloropyridazine-3-carbonitrile (2.8 g, 20.07 mmol, 1 eq) and allyltributylstannane (7.97 g, 24.08 mmol, 7.38 mL, 1.2 eq) in dioxane (30 mL) was added dichloropalladiμm; triphenylphosphane (1.41 g, 2.01 mmol, 0.1 eq) in one portion under N₂. The mixture was stirred at 80° C. for 2 hr. The mixture was then poured into water (100 mL). The aqueous phase was extracted with ethyl acetate (60 mL×2). The combined organic phase was washed with brine (50 mL×2), dried with anhydrous Na2SO4, filtered and concentrated under vacuum. The aqueous phase was quenched by a saturated solution of potassium fluoride (500 mL) and stirred at 20° C. for 2 hr. The residue was purified by flash silica gel chromatography (ISCO®; 40 g SepaFlash® Silica Flash Column, Eluent of 10-15% Ethyl acetate/Petroleum ether gradient @60 mL/min). The purified solution was concentrated to afford the 6-allylpyridazine-3-carbonitrile (780 mg, 27% yield) as a red oil.

LCMS: Rt=0.252 min; (ESI positive ion) m/z: 146.2 (M+H)+(calculated: 146.06).

1H NMR (400 MHz, DMSO-d6) δ=8.29 (d, J=8.7 Hz, 1H), 7.88 (d, J=8.7 Hz, 1H), 6.15-6.02 (m, 1H), 5.24-5.14 (m, 2H), 3.84 (td, J=1.3, 6.7 Hz, 2H).

Ozone was bubbled into a solution of the 6-allylpyridazine-3-carbonitrile (400 mg, 2.76 mmol, 1 eq) in DCM (20 mL), MeOH (20 mL) at −70° C. for 30 minutes. After excess ozone was purged by N₂, and NaBH₄ (521.25 mg, 13.78 mmol, 5 eq) was added at −70° C. The reaction was stirred at this temperature for 60 min, and then warmed and stirred at 0° C. for 60 min. The mixture was poured into water (60 mL). The aqueous phase was extracted with DCM (30 mL×3). The combined organic phase was washed with brine (20 mL×2), dried with anhydrous Na₂SO₄, filtered and concentrated under vacuum. The resultant residue was purified by Prep-TLC (SiO₂, Petroleum ether/Ethyl acetate=0/1). The purified solution was concentrated to afford 6-(2-hydroxyethyl)pyridazine-3-carbonitrile (20 mg, 5% yield) as a colorless oil.

1H NMR (400 MHz, CDCl₃) δ=7.79 (d, J=8.6 Hz, 1H), 7.62 (d, J=8.6 Hz, 1H), 4.16 (t, J=5.7 Hz, 2H), 3.32 (t, J=5.7 Hz, 2H).

### 4,4,5,5-tetramethyl-2-(7-nitro-3,4-dihydronaphthalen-2-yl)-1,3,2-dioxaborolane

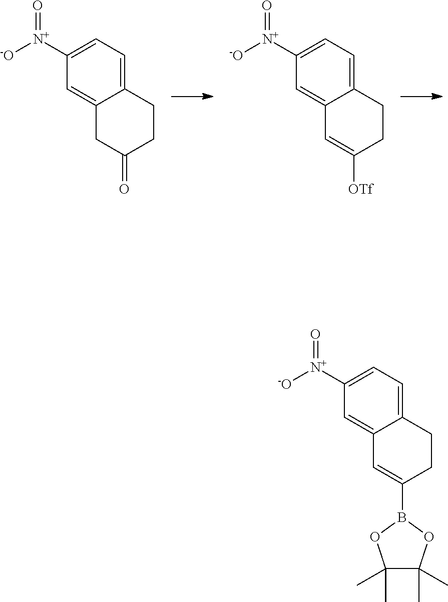

To a solution of 7-nitro-3,4-dihydronaphthalen-2(1H)-one (500 mg, 2.62 mmol, 1 eq) and 1,1,1-trifluoro-N-phenyl-N-(trifluoromethylsulfonyl)methanesulfonamide (1.03 g, 2.88 mmol, 1.1 eq) in THE (10 mL) was added potassium bis(trimethylsilyl)amide (1 M, 3.92 mL, 1.5 eq) (in hexane) at −70° C. under N₂. The mixture was stirred and warmed to 25° C. over the course of 1.5 hr. The reaction mixture was quenched by addition of a solution of 1N HCl (50 mL) at 0° C., and then extracted with ethyl acetate (40 mL×2). The combined organic layers were washed with water (50 mL), dried over Na₂SO₄, filtered and concentrated under reduced pressure to give a residue. The residue was purified by reversed-phase HPLC (0.1% FA condition) to afford 7-nitro-3,4-dihydronaphthalen-2-yl trifluoromethanesulfonate (620 mg) as a yellow oil. 1H NMR (400 MHz, CDCl₃) δ=8.10-8.04 (m, 1H), 7.96 (d, J=2.4 Hz, 1H), 7.32 (d, J=8.4 Hz, 1H), 6.57 (s, 1H), 3.18 (t, J=8.4 Hz, 2H), 2.84-2.72 (m, 2H).

To a solution of 7-nitro-3,4-dihydronaphthalen-2-yl trifluoromethanesulfonate (620 mg, 1.92 mmol, 1 eq), 4,4,4′,4′,5,5,5′,5′-octamethyl-2,2′-bi(1,3,2-dioxaborolane) (633.18 mg, 2.49 mmol, 1.3 eq) and potassium acetate (564.71 mg, 5.75 mmol, 3 eq) in dioxane (20 mL) was added Pd(dppf)Cl2 (140.35 mg, 191.81 μmol, 0.1 eq) under N₂. The mixture was stirred at 80° C. for 16 hr under N₂. The reaction mixture was concentrated under reduced pressure to give a residue. The residue was purified by column chromatography (SiO₂, Petroleum ether/Ethyl acetate=1/0 to 1/1) to afford 4,4,5,5-tetramethyl-2-(7-nitro-3,4-dihydronaphthalen-2-yl)-1,3,2-dioxaborolane (490 mg, 85% yield) as a yellow oil.

1H NMR (400 MHz, CDCl₃) δ=8.02 (dd, J=2.4, 8.1 Hz, 1H), 7.94 (d, J=2.4 Hz, 1H), 7.24 (br d, J=6.4 Hz, 2H), 2.85 (t, J=8.4 Hz, 2H), 2.48-2.43 (m, 2H), 1.33 (s, 12H).

### 3-(4-nitrophenyl)azetidine

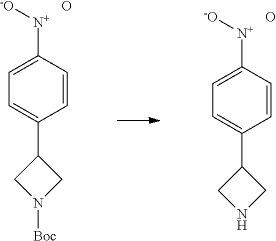

A mixture of tert-butyl 3-(4-nitrophenyl)azetidine-1-carboxylate (100 mg, 359.32 mol, 1 eq) and TsOH (309.38 mg, 1.80 mmol, 5 eq) in EtOAc (10 mL) was degassed and purged with N₂ 3 times. The mixture was stirred at 60° C. for 1 hr under N₂ atmosphere. The mixture was filtered and the solid was collected to afford 3-(4-nitrophenyl)azetidine (90 mg, 71% yield, TsOH salt) as a yellow solid.

1H NMR (400 MHz, DMSO-d6) δ=9.03-8.73 (m, 1H), 8.65-8.39 (m, 1H), 8.27 (d, J=8.6 Hz, 2H), 7.69 (d, J=8.6 Hz, 2H), 7.47 (d, J=8.2 Hz, 2H), 7.11 (d, J=7.8 Hz, 2H), 4.39-4.24 (m, 3H), 4.18-4.09 (m, 2H), 2.28 (s, 3H).

### 2,6-difluoro-4-(2-hydroxyethyl)benzonitrile

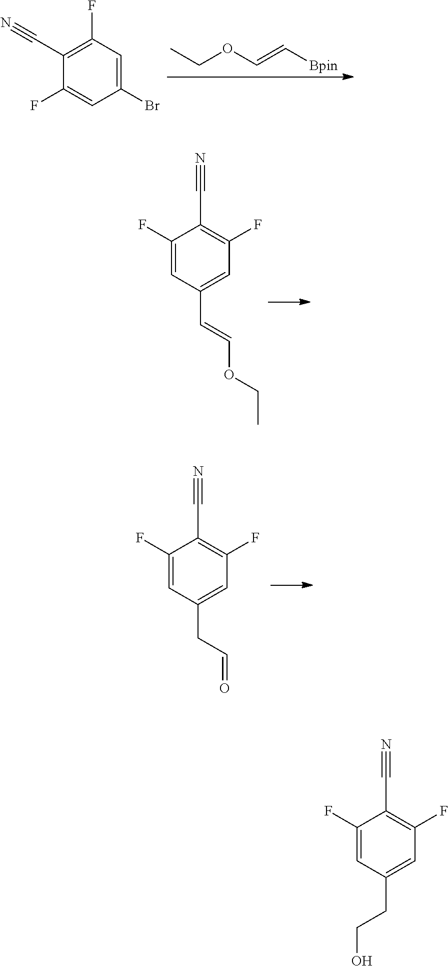

A mixture of 4-bromo-2,6-difluorobenzonitrile (2 g, 9.17 mmol, 1), (E)-2-(2-ethoxyvinyl)-4,4,5,5-tetramethyl-1,3,2-dioxaborolane (2.73 g, 13.76 mmol, 1.5 eq), Pd(dppf)Cl₂·CH₂Cl₂ (749.22 mg, 917.44 μmol, 0.1 eq), and Na₂CO₃ (1.94 g, 18.35 mmol, 2 eq) in dioxane (20 mL) and H₂O (4 mL) was degassed and purged with N₂ 3 times. Then the mixture was stirred at 80° C. for 16 hr under N₂ atmosphere. The reaction mixture was partitioned between H₂O (100 mL) and EtOAc (50 mL). The organic phase was separated, washed with brine (50 mL), dried over Na₂SO₄, filtered and concentrated under reduced pressure. The resultant residue was purified by flash silica gel chromatography (ISCO®; 20 g SepaFlash® Silica Flash Column, Eluent of 0-10% Ethyl acetate/Petroleum ether gradient @40 mL/min) to afford the corresponding enol ether compound (0.9 g, 47% yield) as a yellow oil.

A mixture of the above enol ether compound (400 mg, 1.91 mmol, 1 eq) and TsOH (493.91 mg, 2.87 mmol, 1.5 eq) in acetone (10 mL) was degassed and purged with N₂ 3 times Then the mixture was stirred at 40° C. for 16 hr under N₂ atmosphere. The mixture was poured into ice-H₂O (100 mL). The mixture was extracted with EtOAc (50 mL×2). The combined organic phase was washed with brine (100 mL), dried with anhydrous Na₂SO₄, filtered and concentrated under vacuum to afford the corresponding aldehyde compound (340 mg, crude) as a yellow oil, which was used directly for next step.

To a solution of the above aldehyde compound (340 mg, 1.88 mmol, 1 eq) in EtOH (10 mL) was added NaBH₄ (142.01 mg, 3.75 mmol, 2 eq) at 0° C. The mixture was stirred at 20° C. for 0.5 hr. The mixture was poured into ice-H₂O (50 mL). The mixture was then extracted with EtOAc (20 mL×2). The combined organic phase was washed with brine (50 mL), dried with anhydrous Na₂SO₄, filtered and concentrated under vacuum. The residue was purified by Prep-TLC (Petroleum ether/Ethyl acetate=3/1) to afford 2,6-difluoro-4-(2-hydroxyethyl)benzonitrile (100 mg, 29% yield) as a yellow oil.

1H NMR (400 MHz, CDCl₃) δ=6.98 (d, J=8.2 Hz, 2H), 3.93 (t, J=6.2 Hz, 2H), 2.93 (t, J=6.2 Hz, 2H).

### 7-nitro-1,2,3,4-tetrahydronaphthalen-2-ol

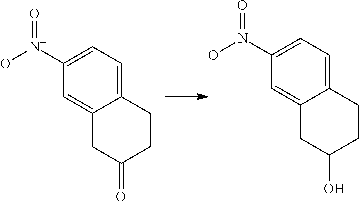

To a solution of 7-nitro-3,4-dihydronaphthalen-2(1H)-one (100 mg, 523.06 μmol, 1 eq) in EtOH (2 mL) was added NaBH₄ (29.68 mg, 784.59 μmol, 1.5 eq) at 0° C. under N₂. The mixture was stirred at 25° C. for 2 hr. The reaction mixture was quenched by the addition of a solution of 1N HCl (10 mL) at 0° C., and then extracted with ethyl acetate (8 mL×2). The combined organic layers were washed with water (10 mL), dried over Na₂SO₄, filtered and concentrated under reduced pressure to give a residue. The residue was purified by column chromatography (SiO₂, Petroleum ether/Ethyl acetate=1/0 to 3/1, Petroleum ether:Ethyl acetate=3/1, Rf=0.4) to afford 7-nitro-1,2,3,4-tetrahydronaphthalen-2-ol (90 mg, 89% yield) as a white solid.

1H NMR (400 MHz, CDCl₃) δ=7.99-7.95 (m, 2H), 7.25 (d, J=8.8 Hz, 1H), 4.29-4.25 (m, 1H), 3.23-3.03 (m, 2H), 2.94-2.84 (m, 2H), 2.13-1.86 (m, 2H), 1.66-1.59 (m, 1H).

### 2-fluoro-4-(2-hydroxyethyl)benzonitrile

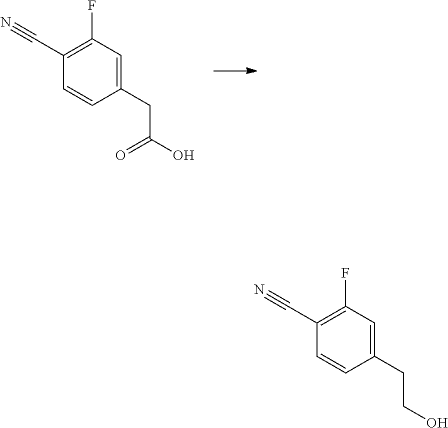 [SMILES: N#Cc1ccc(CC(=O)O)cc1F ; N#Cc1ccc(CCO)cc1F]

To a solution of 2-(4-cyano-3-fluorophenyl)acetic acid (100 mg, 558.20 μmol, 1 eq) in THE (2 mL) was added borane dimethyl sulfide complex (10 M, 61.40 μL, 1.1 eq) dropwise at 0° C. The mixture was stirred at 20° C. for 16 hr under N₂ balloon. To the reaction mixture cooled to 0° C. was slowly added MeOH (20 mL) and stirred for 30 min. Then the mixture was stirred at 65° C. for 1 hr. The solvent was removed under reduced pressure to give a residue, which was purified by Prep-TLC (SiO₂, PE:EtOAc=1:1) to afford 2-fluoro-4-(2-hydroxyethyl)benzonitrile (70 mg, 76% yield) as a colorless oil.

1H NMR (400 MHz, DMSO) δ=7.82 (br t, J=7.3 Hz, 1H), 7.41 (br d, J=11.0 Hz, 1H), 7.28 (br d, J=8.1 Hz, 1H), 4.76 (br d, J=2.2 Hz, 1H), 3.68-3.61 (m, 2H), 2.87-2.76 (m, 2H).

### 2-(6-nitropyridin-3-yl)ethan-1-ol

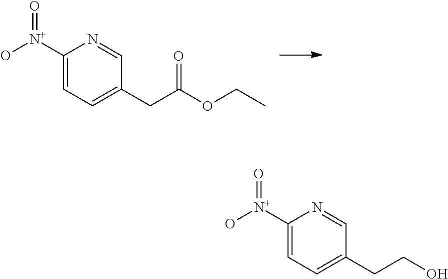

To a solution of ethyl 2-(6-nitropyridin-3-yl)acetate (300 mg, 1.43 mmol, 1 eq) in THE (4 mL) was added LiBH₄ (2 M, 1.07 mL, 1.5 eq) dropwise at 0° C. The mixture was stirred at 0° C. for 1 hr under nitrogen atmosphere. The mixture was slowly poured into water (20 mL) under ice-water bath cooling. The mixture was extracted with DCM (10 mL×2). The combined organic phase was washed with brine (20 mL), dried with anhydrous Na₂SO₄, filtered, and concentrated under vacuum. The resultant residue was purified by Prep-TLC (SiO₂, PE:EtOAc=0:1) to afford 2-(6-nitropyridin-3-yl)ethan-1-ol (70 mg, 29% yield) as a green oil.

1H NMR (400 MHz, CDCl₃) δ=8.54 (s, 1H), 8.22 (d, J=8.2 Hz, 1H), 7.95 (dd, J=1.8, 8.2 Hz, 1H), 3.97 (t, J=6.2 Hz, 2H), 3.03 (t, J=6.2 Hz, 2H).

### 1-(4-(difluoromethoxy)phenyl)ethan-1-ol

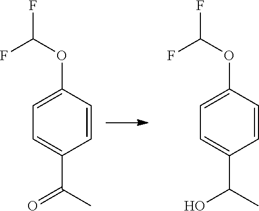

To a solution of 1-(4-(difluoromethoxy)phenyl)ethan-1-one (500 mg, 2.69 mmol, 1 eq) in EtOH (10 mL) was added NaBH₄ (304.85 mg, 8.06 mmol, 3 eq) at 0° C. The mixture was stirred at 0° C. for 2 hr. The mixture was then poured into ice-water (50 mL) and extracted with EtOAc (20 mL×2). The combined organic phase was washed with brine (50 mL), dried with anhydrous Na₂SO₄, filtered and concentrated under vacuum. The residue was purified by column chromatography (SiO₂, Petroleum ether/Ethyl acetate=100/1 to 5/1) to afford 1-(4-(difluoromethoxy)phenyl)ethan-1-ol (490 mg, 97% yield) as a colorless oil.

### 3-fluoro-5-(2-hydroxyethyl)picolinonitrile

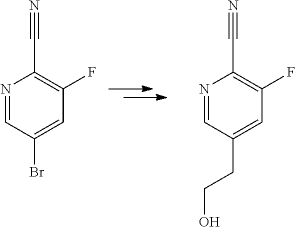 [SMILES: N#Cc1ncc(Br)cc1F ; N#Cc1ncc(CCO)cc1F]

3-fluoro-5-(2-hydroxyethyl)picolinonitrile was synthesized from 5-bromo-3-fluoropicolinonitrile following the protocols described for 2,6-difluoro-4-(2-hydroxyethyl)benzonitrile.

1H NMR (400 MHz, DMSO-d6) δ=8.52 (d, J=1.5 Hz, 1H), 7.99 (dd, J=0.8, 10.2 Hz, 1H), 4.80 (t, J=5.3 Hz, 1H), 3.71-3.65 (m, 2H), 2.87 (t, J=6.3 Hz, 2H).

### 2-(4-(difluoromethyl)phenyl)ethan-1-ol

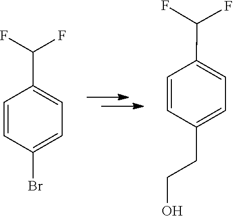 [SMILES: FC(F)c1ccc(Br)cc1 ; OCCc1ccc(C(F)F)cc1]

2-(4-(difluoromethyl)phenyl)ethan-1-ol was synthesized from 1-bromo-4-(difluoromethyl)benzene following the protocols described for 2,6-difluoro-4-(2-hydroxyethyl)benzonitrile.

1H NMR (400 MHz, CDCl₃) δ=7.47 (d, J=7.9 Hz, 2H), 7.34 (d, J=7.8 Hz, 2H), 6.81-6.46 (m, 1H), 3.90 (t, J=6.5 Hz, 2H), 2.93 (t, J=6.5 Hz, 2H).

### 2-(4-chloro-3,5-difluorophenyl)ethan-1-ol

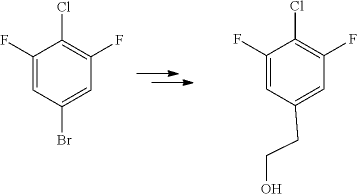 [SMILES: Fc1cc(Br)cc(F)c1Cl ; OCCc1cc(F)c(Cl)c(F)c1]

2-(4-chloro-3,5-difluorophenyl)ethan-1-ol was synthesized from 5-bromo-2-chloro-1,3-difluorobenzene following the protocols described for 2,6-difluoro-4-(2-hydroxyethyl)benzonitrile.

1H NMR (400 MHz, CDCl₃) δ=6.94-6.85 (m, 2H), 3.88 (t, J=6.4 Hz, 2H), 2.85 (t, J=6.4 Hz, 2H).

### 2-(4-(difluoromethoxy)phenyl)ethan-1-ol

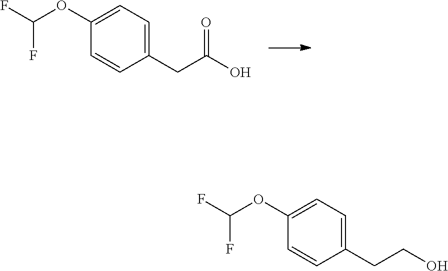 [SMILES: O=C(O)Cc1ccc(OC(F)F)cc1 ; OCCc1ccc(OC(F)F)cc1]

2-(4-(difluoromethoxy)phenyl)ethan-1-ol was synthesized from 2-(4-(difluoromethoxy)phenyl)acetic acid following the protocols described for 2-fluoro-4-(2-hydroxyethyl)benzonitrile.

### 2-(6-(difluoromethoxy)pyridin-3 yl)ethan-1-ol

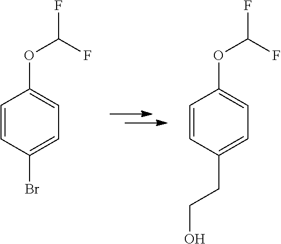 [SMILES: FC(F)Oc1ccc(Br)cc1 ; OCCc1ccc(OC(F)F)cc1]

2-(6-(difluoromethoxy)pyridin-3-yl)ethan-1-ol was synthesized 5-bromo-2-(difluoromethoxy)pyridine following the protocols described for 2,6-difluoro-4-(2-hydroxyethyl)benzonitrile.

LCMS: Rt=0.514 min; (ESI positive ion) m/z: 190.2 (M+H)+(calculated: 190.06).

1H NMR (400 MHz, DMSO-d6) δ=8.09 (d, J=2.1 Hz, 1H), 7.76 (t, J=73.2 Hz, 1H), 7.78 (dd, J=2.3, 8.3 Hz, 1H), 7.00 (d, J=8.3 Hz, 1H), 4.70 (t, J=5.2 Hz, 1H), 3.63-3.56 (m, 2H), 2.71 (t, J=6.6 Hz, 2H).

### 4-(fluoromethoxy)phenol

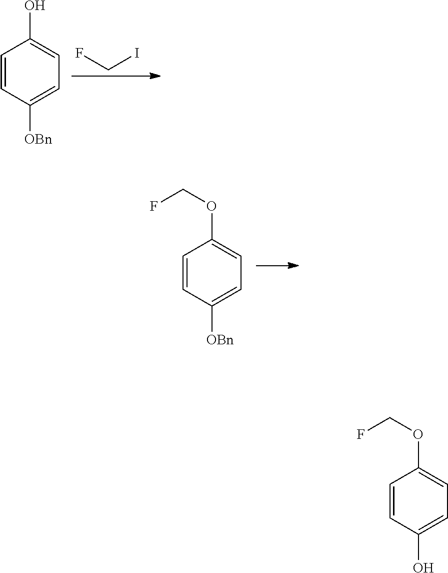

To a mixture of 4-(benzyloxy)phenol (100 mg, 499.42 μmol, 1 eq) and Cs₂CO₃ (195.26 mg, 599.30 μmol, 1.2 eq) in MeCN (5 mL) was added fluoroiodomethane (159.74 mg, 998.84 μmol, 2 eq). The mixture was stirred at 20° C. for 6 hr. The mixture was filtered and concentrated under vacuum. The residue was purified by Prep-TLC (Petroleum ether/Ethyl acetate=3/1) to afford 1-(benzyloxy)-4-(fluoromethoxy)benzene (115 mg, 99% yield) as a white solid.

A mixture of 1-(benzyloxy)-4-(fluoromethoxy)benzene (115 mg, 495.16 μmol, 1 eq) and Pd/C (20 mg, Pd 10%) in MeOH (10 mL) was degassed and purged with H2 (15 psi) 3 times, and then the mixture was stirred at 20° C. for 16 hr under H2 (15 psi) atmosphere. The mixture was filtered and concentrated under vacuum to afford 4-(fluoromethoxy)phenol (68 mg, 97% yield) as a white solid.

1H NMR (400 MHz, CDCl₃) δ=6.99 (d, J=8.8 Hz, 2H), 6.83-6.75 (m, 2H), 5.74-5.55 (m, 2H), 4.63 (s, 1H).

### 2-((4-chlorobenzyl)oxy)acetic acid

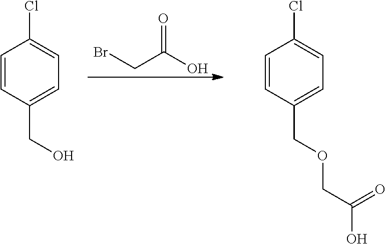 [SMILES: OCc1ccc(Cl)cc1 ; O=C(O)CBr ; O=C(O)COCc1ccc(Cl)cc1]

To a solution of (4-chlorophenyl)methanol (1 g, 7.01 mmol, 1 eq) in DMF (10 mL) was added NaH (420.77 mg, 10.52 mmol, 60% purity, 1.5 eq) at 0° C. The mixture was stirred at 0° C. for 0.5 hr. 2-bromoacetic acid (1.07 g, 7.71 mmol, 555.42 μL, 1.1 eq) was then added to the mixture, and the mixture was stirred at 20° C. for 1 hr. The residue was poured into ice cold solution of 0.5 N HCl (100 mL) and stirred for 1 min. The aqueous phase was extracted with ethyl acetate (50 mL×2). The combined organic phase was washed with brine (100 mL), dried with anhydrous Na₂SO₄, filtered and concentrated under vacuum to afford 2-((4-chlorobenzyl)oxy)acetic acid (650 mg, 3.24 mmol, 46.20% yield) as a white solid.

1H NMR (400 MHz, CDCl₃) δ=9.23-8.31 (m, 1H), 7.37-7.33 (m, 2H), 7.30 (s, 2H), 4.62 (s, 2H), 4.16 (s, 2H).

### 2-(4-(fluoromethyl)phenyl)ethan-1-ol

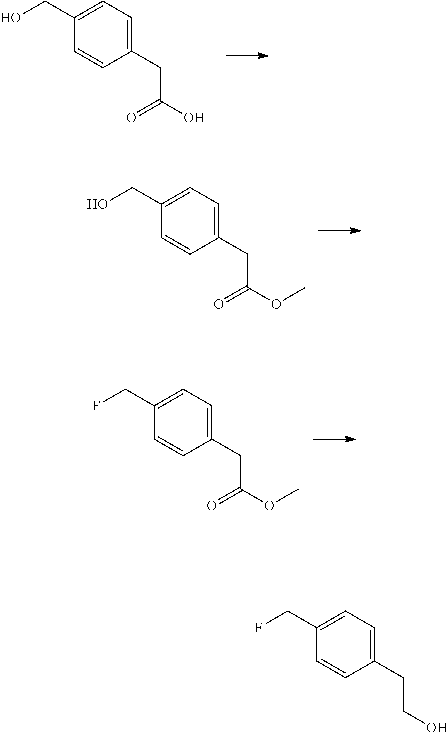

To a solution of 2-(4-(hydroxymethyl)phenyl)acetic acid (2 g, 12.04 mmol, 1 eq) in MeOH (70 mL) was added acetyl chloride (1.89 g, 24.07 mmol, 1.72 mL, 2 eq). The mixture was stirred at 20° C. for 12 hr. The solvent was removed under reduced pressure to afford methyl 2-(4-(hydroxymethyl)phenyl)acetate (2.07 g, crude) as a colorless oil. 1H NMR (400 MHz, DMSO-d6) δ=7.27-7.25 (m, 2H), 7.22-7.18 (m, 2H), 5.16 (t, J=5.6 Hz, 1H), 4.47 (d, J=5.5 Hz, 2H), 3.65 (s, 2H), 3.60 (s, 3H).

To a solution of methyl 2-(4-(hydroxymethyl)phenyl)acetate (1800 mg, 9.99 mmol, 1 eq) in DCM (36 mL) was added sulfur trifluoride (2.42 g, 14.98 mmol, 1.98 mL, 1.5 eq) dropwise at 0° C. The mixture was stirred at 20° C. for 16 hr under nitrogen atmosphere. The reaction mixture was then slowly poured into aqueous NaHCO₃ (100 mL) under ice-water bath cooling. The aqueous phase was extracted with DCM (60 mL×2) and the combined organic phases were washed with brine, dried over anhydrous Na₂SO₄ and concentrated to give a residue. The residue was purified by flash silica gel chromatography (ISCO®; 20 g SepaFlash® Silica Flash Column, Eluent of 0-10% Ethyl acetate/Petroleum ether gradient @50 mL/min) to afford methyl 2-(4-(fluoromethyl)phenyl)acetate (770 mg, 42% yield) as a colorless oil.

1H NMR (400 MHz, DMSO-d6) δ=7.40-7.35 (m, 2H), 7.33-7.29 (m, 2H), 5.45 (s, 1H), 5.33 (s, 1H), 3.71 (s, 2H), 3.61 (s, 3H).

To a solution of methyl 2-(4-(fluoromethyl)phenyl)acetate (200 mg, 1.10 mmol, 1 eq) in THE (4 mL) was added LiBH₄ (2 M, 823.31 μL, 1.5 eq) dropwise at 0° C. The mixture was stirred at 20° C. for 16 hr under nitrogen atmosphere. The mixture was slowly poured into a saturated aqueous solution of NH₄Cl (20 mL) under ice-water bath cooling. The mixture was then extracted with EtOAc (10 mL×2). The combined organic phase was washed with brine (20 mL), dried with anhydrous Na₂SO₄, filtered and concentrated under vacuum. The residue was purified by Prep-TLC (SiO₂, PE:EtOAc=2:1) to afford 2-(4-(fluoromethyl)phenyl)ethan-1-ol (121 mg, 71% yield) as a colorless oil.

1H NMR (400 MHz, DMSO-d6) δ=7.35-7.30 (m, 2H), 7.28-7.23 (m, 2H), 5.42 (s, 1H), 5.30 (s, 1H), 4.66 (t, J=5.3 Hz, 1H), 3.60 (dt, J=5.3, 7.0 Hz, 2H), 2.73 (t, J=6.7 Hz, 2H).

### 5-(2-hydroxyethyl)picolinonitrile

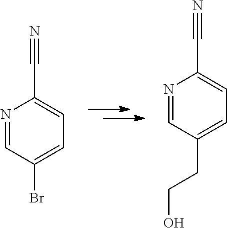 [SMILES: N#Cc1ccc(Br)cn1 ; N#Cc1ccc(CCO)cn1]

5-(2-hydroxyethyl)picolinonitrile was synthesized from the 5-bromopicolinonitrile following the protocols described for 2,6-difluoro-4-(2-hydroxyethyl)benzonitrile.

1H NMR (400 MHz, CDCl₃) δ=8.61 (d, J=1.5 Hz, 1H), 7.75 (dd, J=2.1, 7.9 Hz, 1H), 7.65 (d, J=7.9 Hz, 1H), 3.94 (t, J=6.2 Hz, 2H), 2.95 (t, J=6.3 Hz, 2H).

### 5-bromo-2-(fluoromethoxy)pyridine

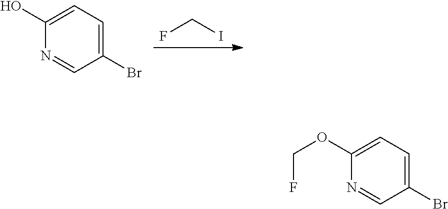

To a mixture of 5-bromopyridin-2-ol (1 g, 5.75 mmol, 1 eq) in ACN (20 mL) were added silver carbonate (1.90 g, 6.90 mmol, 312.79 μL, 1.2 eq) and fluoroiodomethane (1.10 g, 6.90 mmol, 1.2 eq). The mixture was stirred at 80° C. for 12 hr. The reaction mixture was filtered and concentrated to give a residue. The residue was purified by flash silica gel chromatography (ISCO®; 12 g SepaFlash® Silica Flash Column, Eluent of 0-8% Ethyl acetate/Petroleum ether gradient @50 mL/min). The purified solution was concentrated to afford 5-bromo-2-(fluoromethoxy)pyridine (670 mg, 56% yield) as a colorless oil.

1H NMR (400 MHz, DMSO-d6) δ=8.38 (d, J=2.4 Hz, 1H), 8.06 (dd, J=2.6, 8.8 Hz, 1H), 7.01 (d, J=8.8 Hz, 1H), 6.12 (s, 1H), 5.99 (s, 1H).

### 2-(6-(fluoromethoxy)pyridin-3 yl)ethan-1-ol

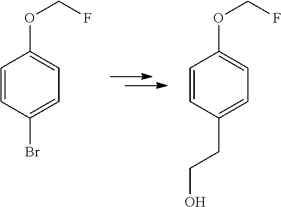 [SMILES: FCOc1ccc(Br)cc1 ; OCCc1ccc(OCF)cc1]

2-(6-(fluoromethoxy)pyridin-3-yl)ethan-1-ol was synthesized from the 5-bromo-2-(fluoromethoxy)pyridine following the protocols described for 2,6-difluoro-4-(2-hydroxyethyl)benzonitrile.

1H NMR (400 MHz, DMSO-d6) δ=8.07 (d, J=2.1 Hz, 1H), 7.70 (dd, J=2.4, 8.4 Hz, 1H), 6.90 (d, J=8.3 Hz, 1H), 6.13 (s, 1H), 5.99 (s, 1H), 4.67 (t, J=5.1 Hz, 1H), 3.63-3.54 (m, 2H), 2.69 (t, J=6.7 Hz, 2H).

### 2-(4-(fluoromethoxy)phenyl)ethan-1-ol

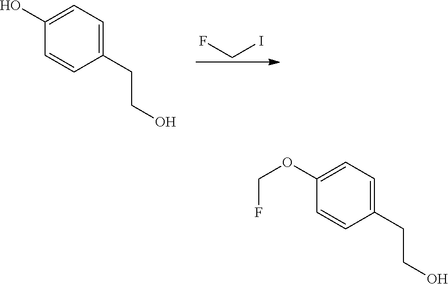

To a mixture of 4-(2-hydroxyethyl)phenol (170 mg, 1.23 mmol, 1 eq) in ACN (4 mL) were added Cs₂CO₃ (481.08 mg, 1.48 mmol, 1.2 eq) and fluoroiodomethane (236.14 mg, 1.48 mmol, 1.2 eq). The mixture was stirred at 20° C. for 5 hr. The reaction mixture was then added to water (30 mL) and extracted with EtOAc (20 mL×2). The organic layer was washed with brine and dried by Na₂SO₄. The solution was concentrated to give a residue, which was purified by column chromatography (SiO₂, Petroleum ether/Ethyl acetate=5/1 to 2/1) to afford 2-(4-(fluoromethoxy)phenyl)ethan-1-ol (208 mg, 99% yield) as a colorless oil.

1H NMR (400 MHz, DMSO-d6) δ=7.20 (d, J=8.6 Hz, 2H), 7.00 (d, J=8.4 Hz, 2H), 5.92-5.70 (m, 2H), 4.63-4.59 (m, 1H), 3.57 (dt, J=5.3, 7.0 Hz, 2H), 2.68 (t, J=7.0 Hz, 2H).

### 5-(2-hydroxyethyl)pyrimidine-2-carbonitrile

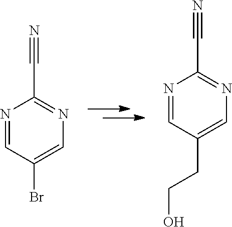 [SMILES: N#Cc1ncc(Br)cn1 ; N#Cc1ncc(CCO)cn1]

5-(2-hydroxyethyl)pyrimidine-2-carbonitrile was synthesized from the 5-bromopyrimidine-2-carbonitrile following the protocols described for 2,6-difluoro-4-(2-hydroxyethyl)benzonitrile.

LCMS: Rt=0.241 min; (ESI positive ion) m/z: 149.7 (M+H)+(calculated: 150.06).

### 2-(6-(trifluoromethoxy)pyridin-3-yl)ethan-1-ol

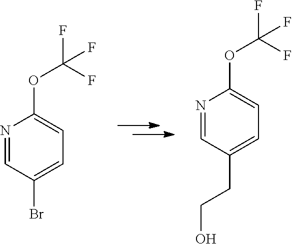 [SMILES: FC(F)(F)Oc1ccc(Br)cn1 ; OCCc1ccc(OC(F)(F)F)nc1]

2-(6-(trifluoromethoxy)pyridin-3-yl)ethan-1-ol was synthesized from the 5-bromo-2-(trifluoromethoxy)pyridine following the protocols described for 2,6-difluoro-4-(2-hydroxyethyl)benzonitrile.

### 5-bromo-2-(fluoromethoxy)pyrimidine

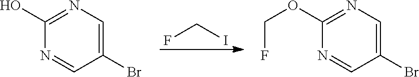

5-bromo-2-(fluoromethoxy)pyrimidine was synthesized from the 5-bromopyrimidin-2-ol following the protocols described for 5-bromo-2-(fluoromethoxy)pyridine.

### 2-(2-(fluoromethoxy)pyrimidin-5-yl)ethan-1-ol

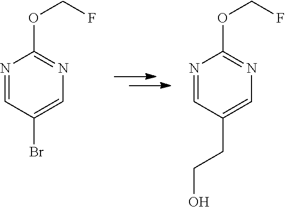 [SMILES: FCOc1ncc(Br)cn1 ; OCCc1cnc(OCF)nc1]

2-(2-(fluoromethoxy)pyrimidin-5-yl)ethan-1-ol was synthesized from the 5-bromo-2-(fluoromethoxy)pyrimidine following the protocols described for 2,6-difluoro-4-(2-hydroxyethyl)benzonitrile.

1H NMR (400 MHz, CDCl₃) δ=8.50 (s, 2H), 6.22-5.99 (m, 2H), 3.89 (t, J=6.2 Hz, 2H), 2.84 (t, J=6.2 Hz, 2H).

### 2-(((1R,4R)-4-(trifluoromethyl)cyclohexyl)oxy)acetic acid

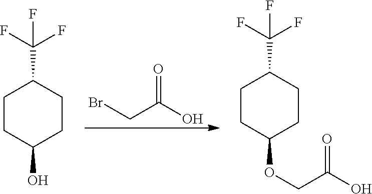

To a solution of (1R,4R)-4-(trifluoromethyl)cyclohexan-1-ol (100 mg, 594.68 mol, 1 eq) in DMF (5 mL) was added NaH (35.68 mg, 892.03 μmol, 60% purity, 1.5 eq) at 0° C. The mixture was stirred at 0° C. for 0.5 hr. 2-bromoacetic acid (90.89 mg, 654.15 μmol, 47.09 μL, 1.1 eq) was added to the mixture and the mixture was stirred at 20° C. for 1 hr. The mixture was poured into ice cold solution of 0.5 N HCl (10 mL) and stirred for 1 min. The aqueous phase was extracted with ethyl acetate (5 mL×2). The combined organic phase was washed with brine (10 mL), dried with anhydrous Na₂SO₄, filtered and concentrated under vacuum to afford 2-(((1R,4R)-4-(trifluoromethyl)cyclohexyl)oxy)acetic acid (80 mg, 59% yield) as a white solid.

1H NMR (400 MHz, CDCl₃) δ=4.17 (s, 2H), 3.45-3.32 (m, 1H), 2.19 (br d, J=8.0 Hz, 2H), 2.11-1.96 (m, 3H), 1.43-1.29 (m, 4H).

### 3-(aminomethyl)-N,N-dimethylbenzamide

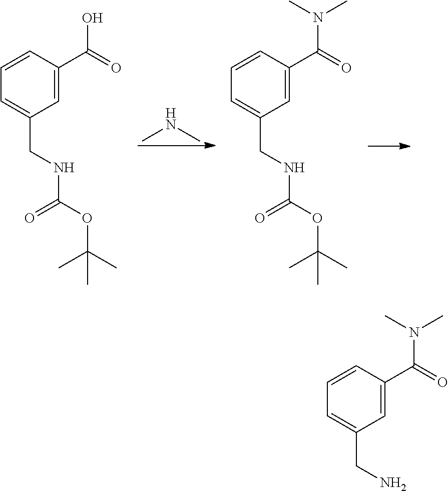

To a solution of 3-(((tert-butoxycarbonyl)amino)methyl)benzoic acid (400 mg, 1.59 mmol, 1 eq) in THE (5 mL) was added di(imidazol-1-yl)methanone (412.51 mg, 2.54 mmol, 1.6 eq). The mixture was stirred at 20° C. for 3 hr. After 3 hr, dimethylamine (544.55 mg, 6.68 mmol, 611.86 μL, 4.2 eq, HCl) and DIEA (410.98 mg, 3.18 mmol, 553.89 μL, 2 eq) were added to the mixture. The mixture was stirred at 60° C. for 16 hr. The reaction mixture was concentrated to give a residue, which was purified by flash silica gel chromatography (ISCO®; 12 g SepaFlash® Silica Flash Column, Eluent of 40-50% Ethyl acetate/Petroleum ether gradient @60 mL/min). The purified solution was concentrated to afford the corresponding amide compound (420 mg, 95% yield) as a white solid.

LCMS: Rt=0.801 min; (ESI positive ion) m/z: 279.2 (M+H)+(calculated: 279.16).

To a solution of the above amide compound (420 mg, 1.51 mmol, 1 eq) in dioxane (3 mL) was added HCl/dioxane (4 M, 5 mL, 13.25 eq) at 0° C. The mixture was stirred at 20° C. for 2 hr. The reaction mixture was concentrated to afford 3-(aminomethyl)-N,N-dimethylbenzamide (300 mg, crude, HCl salt) as a colorless gum.

1H NMR (400 MHz, DMSO-d6) δ=8.51 (br s, 2H), 7.59-7.52 (m, 2H), 7.47 (t, J=7.5 Hz, 1H), 7.42-7.37 (m, 1H), 4.05 (q, J=5.3 Hz, 2H), 3.02-2.88 (m, 6H).

### 2-(6-(difluoromethyl)pyridin-3 yl)ethan-1-ol

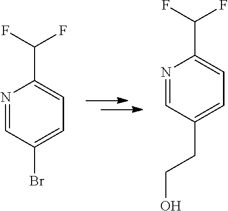 [SMILES: FC(F)c1ccc(Br)cn1 ; OCCc1ccc(C(F)F)nc1]

2-(6-(difluoromethyl)pyridin-3-yl)ethan-1-ol was synthesized from the 5-bromo-2-(difluoromethyl)pyridine following the protocols described above for 2,6-difluoro-4-(2-hydroxyethyl)benzonitrile.

1H NMR (400 MHz, CDCl₃) δ=8.55 (s, 1H), 7.75 (dd, J=1.7, 7.9 Hz, 1H), 7.60 (d, J=7.9 Hz, 1H), 6.83-6.48 (m, 1H), 3.93 (t, J=6.4 Hz, 2H), 2.94 (t, J=6.4 Hz, 2H).

### 5-(2-hydroxyethyl)-1-methylpyridin-2(1H)-one

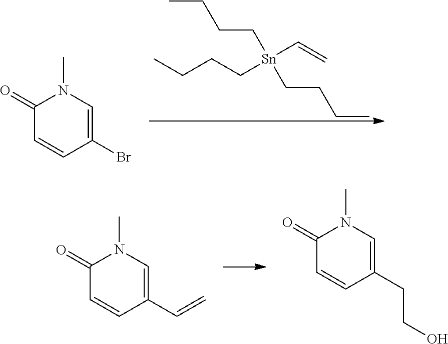

To a mixture of 5-bromo-1-methylpyridin-2(1H)-one (2 g, 10.64 mmol, 1 eq) and tributyl(vinyl)stannane (4.05 g, 12.76 mmol, 3.71 mL, 1.2 eq) in dioxane (10 mL) was added Pd(PPh₃)₂Cl₂ (746.61 mg, 1.06 mmol, 0.1 eq) in one portion under N₂. The mixture was stirred at 110° C. for 2 hours. The mixture was then poured into water (10 mL). The aqueous phase was extracted with ethyl acetate (30 mL×2). The combined organic phase was washed with brine (10 mL×2), dried with anhydrous Na₂SO₄, filtered and concentrated under vacuum. The resultant residue was purified by silica gel chromatography (SiO₂, Petroleum ether/Ethyl acetate=5/1, 1/1) to afford the corresponding vinyl compound (1.1 g, 7.52 mmol, 70.69% yield, 92.4% purity) as a yellow oil.

1H NMR (400 MHz, CDCl₃) δ=7.58 (dd, J=2.4, 9.4 Hz, 1H), 7.23 (d, J=2.4 Hz, 1H), 6.60 (d, J=9.4 Hz, 1H), 6.41 (dd, J=11.0, 17.6 Hz, 1H), 5.47 (d, J=17.6 Hz, 1H), 5.14 (d, J=11.0 Hz, 1H), 3.56 (s, 3H).

To a solution of the above vinyl compound (200 mg, 1.48 mmol, 1 eq) in THE (8 mL) was added borane tetrahydrofuran complex (1 M, 1.78 mL, 1.2 eq) at 0° C. and the mixture was heated to 20° C. for 2 hr. Then the mixture was added an aqueous solution of 2 N sodium hydroxide (1.48 mL, 2 eq) at 0° C. under N₂, followed by hydrogen peroxide (419.43 mg, 3.70 mmol, 355.45 μL, 30% purity, 2.5 eq). The mixture was stirred at 20° C. for 2 hours. The mixture was then poured into water (30 mL) and stirred for 2 min. The aqueous phase was washed with ethyl acetate (30 mL×2). The aqueous phase was then lyophilized to give a white solid. The white solid was washed with DCM/MeOH=10/1 (50 mL). The organic phase was concentrated under vacuum. The residue was purified by Prep-TLC (DCM/MeOH=10/1) to afford 5-(2-hydroxyethyl)-1-methylpyridin-2(1H)-one (80 mg, 35% yield) as a yellow oil.

1H NMR (400 MHz, CDCl₃) δ=7.30 (br d, J=2.6 Hz, 1H), 7.19 (d, J=1.8 Hz, 1H), 6.62 (d, J=9.2 Hz, 1H), 3.81 (t, J=6.2 Hz, 2H), 3.55 (s, 3H), 2.62 (t, J=6.2 Hz, 2H).

### 6-(2-hydroxyethyl)nicotinonitrile

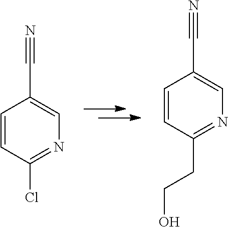 [SMILES: N#Cc1ccc(Cl)nc1 ; N#Cc1ccc(CCO)nc1]

6-(2-hydroxyethyl)nicotinonitrile was synthesized from the 6-chloronicotinonitrile following the protocols described for 2,6-difluoro-4-(2-hydroxyethyl)benzonitrile.

1H NMR (400 MHz, CDCl₃) δ=8.81 (d, J=1.6 Hz, 1H), 7.90 (dd, J=2.1, 8.1 Hz, 1H), 7.34 (d, J=8.1 Hz, 1H), 4.07 (t, J=5.6 Hz, 2H), 3.12 (t, J=5.5 Hz, 2H).

### 5-bromo-2-(fluoromethyl)pyrimidine

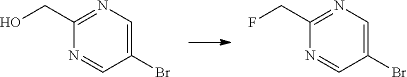 [SMILES: OCc1ncc(Br)cn1 ; FCc1ncc(Br)cn1]

To a solution of (5-bromopyrimidin-2-yl)methanol (1 g, 5.29 mmol, 1 eq) in DCM (14 mL) was added dropwise a solution of diethylaminosulfur trifluoride (1.11 g, 6.88 mmol, 908.74 μL, 1.3 eq) in DCM (14 mL) at −78° C. The mixture was stirred at −78° C. for 1 hr under nitrogen atmosphere. Then the mixture was warmed to 20° C. and stirred at this temperature for 12 hr. The reaction mixture was then slowly poured into a saturated aqueous solution of NaHCO₃ (60 mL) under ice-water bath cooling. The aqueous phase was extracted with DCM (40 mL×3) and the combined organic phases were washed with brine, dried over anhydrous Na₂SO₄ and concentrated to give a residue. The residue was purified by column chromatography (SiO2, Petroleum ether/Ethyl acetate=25/1 to 10/1) to afford 5-bromo-2-(fluoromethyl)pyrimidine (384 mg, 38% yield) as a yellow solid.

LCMS: Rt=0.123 min; (ESI positive ion) m/z: 191.1 (M+H)+(calculated: 190.95).

1H NMR (400 MHz, CDCl₃) δ=8.85 (s, 2H), 5.57 (s, 1H), 5.46 (s, 1H).

### 2-(2-(fluoromethyl)pyrimidin-5-yl)ethan-1-ol

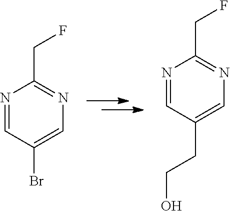 [SMILES: FCc1ncc(Br)cn1 ; OCCc1cnc(CF)nc1]

2-(2-(fluoromethyl)pyrimidin-5-yl)ethan-1-ol was synthesized from the 5-bromo-2-(fluoromethyl)pyrimidine following the protocols described for 2,6-difluoro-4-(2-hydroxyethyl)benzonitrile.

LCMS: Rt=0.123 min; (ESI positive ion) m/z: 157.1 (M+H)+(calculated: 157.07).

### 5-bromo-2-(difluoromethyl)pyrimidine

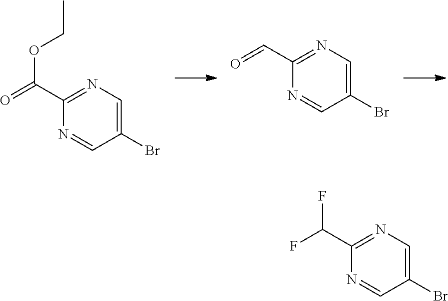

To a solution of ethyl 5-bromopyrimidine-2-carboxylate (2.5 g, 10.82 mmol, 1 eq) in THE (30 mL) was added DIBAL-H (1 M, 17.31 mL, 1.6 eq) at −60° C. The mixture was stirred at −60° C. for 1 hr and then quenched by the addition of a saturated aqueous solution of NH₄Cl (500 mL) and EtOAc (200 mL). An emulsion formed, and a solution of 2N HCl was added to clear the emulsion. The mixture was filtered and the filtrate was extracted with EtOAc (500 mL×2). The combined organic phase was washed with brine (500 mL), dried with anhydrous Na₂SO₄, filtered and concentrated under vacuum to afford 5-bromopyrimidine-2-carbaldehyde (1.6 g, crude) as a yellow solid, which was used directly for next step.

To a solution of 5-bromopyrimidine-2-carbaldehyde (1.6 g, 8.56 mmol, 1 eq) in DCM (40 mL) was added diethylaminosulfur trifluoride (6.90 g, 42.78 mmol, 5.65 mL, 5 eq) at 0° C. The mixture was stirred at 20° C. for 16 hr and then poured into an ice cold saturated aqueous solution of NaHCO₃ (200 mL). The aqueous phase was extracted with DCM (100 mL×2). The combined organic phase was washed with brine (200 mL), dried with anhydrous Na₂SO₄, filtered and concentrated under vacuum. The resultant residue was purified by column chromatography (SiO₂, Petroleum ether/Ethyl acetate=100/1 to 10/1) to afford 5-bromo-2-(difluoromethyl)pyrimidine (950 mg, 52% yield) as a yellow solid.

LCMS: Rt=0.483 min; (ESI positive ion) m/z: 208.6 (M+H)+(calculated: 208.94).

### 2-(2-(difluoromethyl)pyrimidin-5-yl)ethan-1-ol

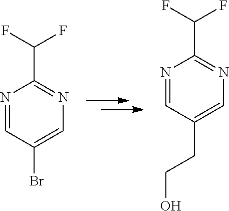 [SMILES: FC(F)c1ncc(Br)cn1 ; OCCc1cnc(C(F)F)nc1]

2-(2-(difluoromethyl)pyrimidin-5-yl)ethan-1-ol was synthesized from the 5-bromo-2-(difluoromethyl)pyrimidine following the protocols described for 2,6-difluoro-4-(2-hydroxyethyl)benzonitrile.

1H NMR (400 MHz, CDCl₃) δ=8.79-8.75 (m, 2H), 6.81-6.50 (m, 1H), 3.97-3.91 (m, 2H), 2.92 (t, J=6.2 Hz, 2H).

### 2-(4-(methylsulfinyl)phenyl)ethan-1-ol

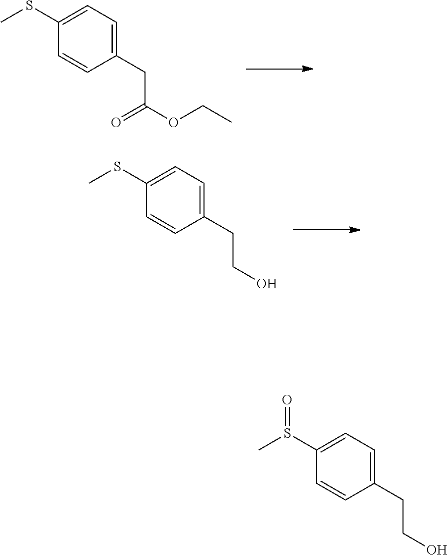

To a solution of ethyl 2-(4-(methylthio)phenyl)acetate (1 g, 4.76 mmol, 1 eq) in THE (10 mL) was added LiBH₄ (2 M, 3.57 mL, 1.5 eq) dropwise at 0° C. The mixture was stirred at 0° C. for 1 hr under N₂ atmosphere. The mixture was slowly poured into water (30 mL) under ice-water bath cooling. The mixture was then extracted with DCM (20 mL×2). The combined organic phase was washed with brine (30 mL), dried with anhydrous Na₂SO₄, filtered and concentrated under vacuum. The residue was purified by column chromatography (SiO₂, Petroleum ether/Ethyl acetate=100/0 to 5/1) to afford 2-(4-(methylthio)phenyl)ethan-1-ol (700 mg, 87% yield) as a yellow gum.

1H NMR (400 MHz, CDCl₃-d) δ=7.25-7.21 (m, 2H), 7.19-7.14 (m, 2H), 3.84 (t, J=6.4 Hz, 2H), 2.83 (t, J=6.8 Hz, 2H), 2.48 (s, 3H).

To a solution of 2-(4-(methylthio)phenyl)ethan-1-ol (300 mg, 1.78 mmol, 1 eq) in CHCl₃ (5 mL) was added meta-chloroperoxybenzoic acid (361.99 mg, 1.78 mmol, 85% purity, 1 eq). The reaction was stirred at 20° C. for 3 hr and then stirred at 20° C. for 12 hr. The reaction mixture was quenched by a saturated aqueous solution of Na₂SO₃ (20 mL), and then diluted with water (10 mL) and then extracted with ethyl acetate (20 mL×2). The combined organic layers were washed with brine (20 mL), dried over Na₂SO₄, filtered and concentrated under reduced pressure to give a residue. The residue was purified by column chromatography (SiO₂, Petroleum ether/Ethyl acetate=100/0 to 0/1) to afford the 2-(4-(methylsulfinyl)phenyl)ethan-1-ol (130 mg, 40% yield) as a yellow oil.

### ((3aR,4R,6R, 6aR)-6-(6-chloro-9H-purin-9-yl)-2,2-dimethyltetrahydrofuro[3,4-d][1,3]dioxol-4-yl)methanol

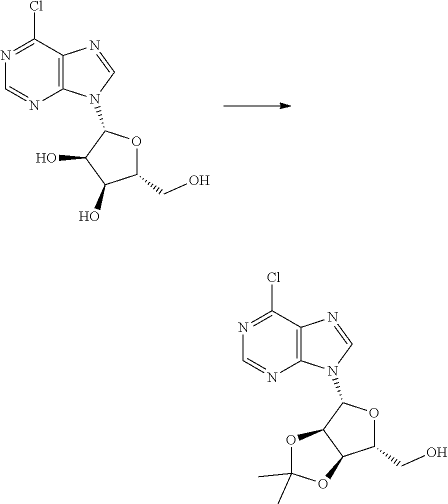 [SMILES: OC[C@H]1O[C@@H](n2cnc3c(Cl)ncnc32)[C@H](O)[C@@H]1O ; CC1(C)O[C@@H]2[C@H](O1)[C@@H](CO)O[C@H]2n1cnc2c(Cl)ncnc21]

To a solution of 6-chloropurine riboside (5 g, 17.44 mmol, 1 eq) in acetone (200 mL) was added TsOH (30.03 g, 174.42 mmol, 10 eq) in one portion. The mixture was stirred at 20° C. for 3 hr. The reaction mixture was then poured into a saturated aqueous solution of NaHCO3 (200 mL) slowly and stirred at 0° C. for 10 min. The volatiles were then removed under reduced pressure and the mixture was extracted with EtOAc (100 mL×3). The organic layer was washed with brine, dried by Na2SO4, and concentrated to afford the corresponding ((3aR,4R,6R,6aR)-6-(6-chloro-9H-purin-9-yl)-2,2-dimethyltetrahydrofuro[3,4-d][1,3]dioxol-4-yl)methanol (5 g, 88% yield) as a white solid.

LCMS: Rt=0.731 min; (ESI positive ion) m/z: 327.10 (M+H)+(calculated: 327.08).

### Intermediate Compound 1:

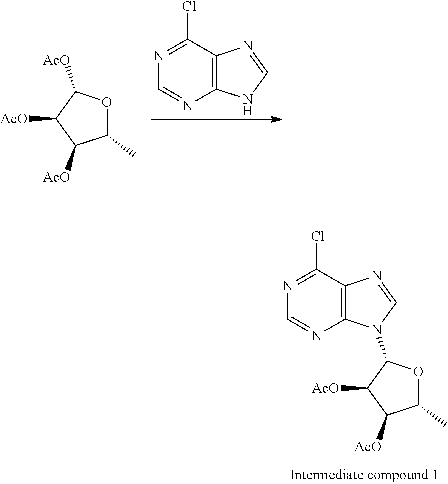 [SMILES: CC(=O)O[C@@H]1O[C@H](C)[C@@H](OC(C)=O)[C@H]1OC(C)=O ; Clc1ncnc2[nH]cnc12 ; CC(=O)O[C@@H]1[C@H](OC(C)=O)[C@@H](C)O[C@H]1n1cnc2c(Cl)ncnc21]

To a solution of 6-chloro-9H-purine (71.27 g, 461.11 mmol, 1.2 eq) in MeCN (1000 mL) was added BSA (156.34 g, 768.52 mmol, 189.96 mL, 2 eq). The mixture was stirred at 20° C. for 3 hr under N₂ atmosphere. After 3 hr, the mixture was concentrated to give a residue and a solution of (2S,3R,4R,5R)-5-methyltetrahydrofuran-2,3,4-triyl triacetate (100 g, 384.26 mmol, 1 eq) in MeCN (1000 mL) was added to the residue. TMSOTf (239.13 g, 1.08 μmol, 194.42 mL, 2.8 eq) was added to the mixture dropwise at 0° C. The mixture was stirred at 20° C. for 12 hr under N₂ atmosphere. The reaction mixture was poured into cold saturated solution of NaHCO₃ (500 mL) and stirred for 1 min, and then extracted with ethyl acetate (1000 mL×3). The combined organic layers were dried with anhydrous Na₂SO₄, filtered and concentrated under reduced pressure to give a residue. The residue was purified by flash silica gel chromatography (ISCO®; 220 g SepaFlash® Silica Flash Column, Eluent of 0-41% Ethyl acetate/Petroleum ether gradient @100 mL/min) to afford Intermediate Compound 1 (118 g, 80% yield) as a white solid. LCMS: Rt=0.309 min; (ESI positive ion) m/z: 355.10 (M+H)+(calculated: 355.07).

1H NMR (400 MHz, CDCl₃) δ=8.75 (s, 1H), 8.25 (s, 1H), 6.11 (d, J=4.8 Hz, 1H), 5.95 (t, J=5.4 Hz, 1H), 5.37 (t, J=5.4 Hz, 1H), 4.36-4.28 (m, 1H), 2.11 (s, 3H), 2.04 (s, 3H), 1.48 (d, J=6.4 Hz, 3H).

### Intermediate Compound 2:

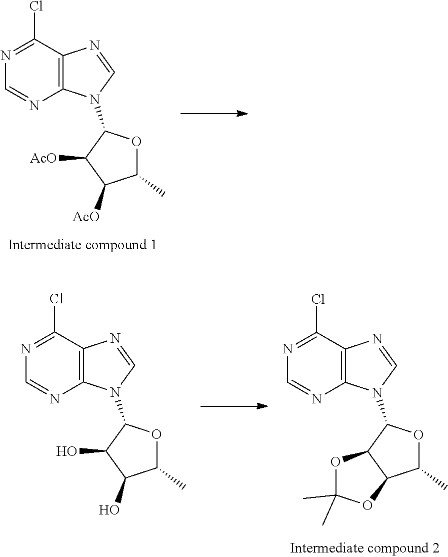 [SMILES: CC(=O)O[C@@H]1[C@H](OC(C)=O)[C@@H](C)O[C@H]1n1cnc2c(Cl)ncnc21 ; C[C@H]1O[C@@H](n2cnc3c(Cl)ncnc32)[C@H](O)[C@@H]1O ; C[C@H]1O[C@@H](n2cnc3c(Cl)ncnc32)[C@@H]2OC(C)(C)O[C@@H]21]

To a solution of Intermediate Compound 1 (11.4 g, 32.14 mmol, 1 eq) in MeOH (100 mL) was added NH₃·H₂O (45.50 g, 363.52 mmol, 50 mL, 28% purity, 11.31 eq) at −40° C. The mixture was stirred at 0° C. for 0.5 hr. The reaction mixture was concentrated under reduced pressure to afford the corresponding diol (8.7 g, crude) as a white solid.

LCMS: Rt=0.235 min; (ESI positive ion) m/z: 270.90 (M+H)+(calculated: 271.05).

To a solution of the diol compound (8.7 g, crude) in acetone (90 mL) was added TsOH·H₂O (61.14 g, 321.42 mmol, 10 eq) at 0° C. The mixture was stirred at 25° C. for 3 hr. The reaction mixture was then poured into a cold saturated solution of NaHCO₃ (500 mL), stirred for 1 min, and extracted with ethyl acetate (1000 mL×2). The combined organic layers were dried with anhydrous Na₂SO₄, filtered and concentrated under reduced pressure to give a residue. The residue was purified by flash silica gel chromatography (ISCO®; 40 g Sepa Flash® Silica Flash Column, Eluent of 0-24% Ethyl acetate/Petroleum ether gradient @100 mL/min) to afford Intermediate Compound 2 (7.8 g, 76% yield) as a colorless oil.

LCMS: Rt=0.416 min; (ESI positive ion) m/z: 311.10 (M+H)+(calculated: 311.08).

### Intermediate Compound 3:

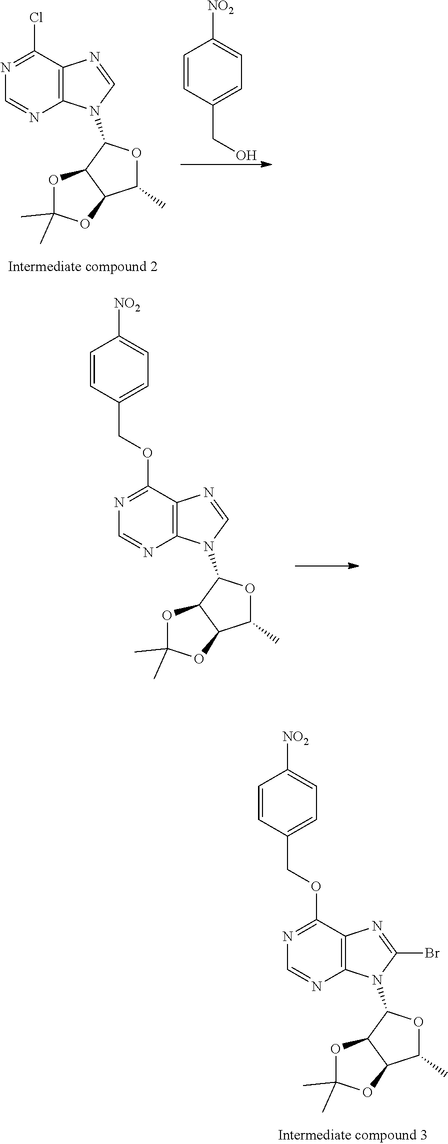 [SMILES: C[C@H]1O[C@@H](n2cnc3c(Cl)ncnc32)[C@@H]2OC(C)(C)O[C@@H]21 ; O=[N+]([O-])c1ccc(CO)cc1 ; C[C@H]1O[C@@H](n2cnc3c(OCc4ccc([N+](=O)[O-])cc4)ncnc32)[C@@H]2OC(C)(C)O[C@@H]21 ; C[C@H]1O[C@@H](n2c(Br)nc3c(OCc4ccc([N+](=O)[O-])cc4)ncnc32)[C@@H]2OC(C)(C)O[C@@H]21]

A mixture of Intermediate Compound 2 (2 g, 6.44 mmol, 1 eq, (4-nitrophenyl)methanol (985.63 mg, 6.44 mmol, 1 eq, Pd₂(dba)₃ (589.39 mg, 643.63 μmol, 0.1 eq, Xantphos (744.84 mg, 1.29 mmol, 0.2 eq and CS2CO₃ (5.24 g, 16.09 mmol, 2.5 eq in toluene (15 mL) was degassed and purged with N₂ 3 times. The mixture was stirred at 80° C. for 1 hr under N₂ atmosphere. The reaction mixture was concentrated under reduced pressure to give a residue, which was purified by flash silica gel chromatography (ISCO®; 40 g SepaFlash® Silica Flash Column, Eluent of 0-50% Ethyl acetate/Petroleum ether gradient @80 mL/min) to afford the corresponding ether (1.05 g, 38% yield) as an orange oil.

LCMS: Rt=0.568 min; (ESI positive ion) m/z: 428.20 (M+H)+(calculated: 428.15).

To a solution of the ether compound (500 mg, 1.17 mmol, 1 eq in THF (5 mL) was added LDA (2 M, 2.92 mL, 5 eq at −70° C. The mixture was stirred at −70° C. for 0.5 hr. A solution of carbon tetrabromide (1.55 g, 4.68 mmol, 4 eq in THF (3 mL) was added to the mixture and the mixture was stirred at 25° C. for 12 hr. The reaction mixture was slowly poured into a saturated aqueous solution of NH₄Cl (100 mL) under ice-water bath cooling. The aqueous phase was extracted with EtOAc (50 mL×3) and the combined organic phases were washed with brine, dried over anhydrous Na₂SO₄ and concentrated to give a residue. The residue was purified by flash silica gel chromatography (ISCO®; 40 g SepaFlash @Silica Flash Column, Eluent of 0-20% Ethyl acetate/Petroleum ether gradient @100 mL/min) to afford Intermediate Compound 3 (230 mg, 20% yield) as a red oil.

LCMS: Rt=0.500 min; (ESI positive ion) m/z: 507.90 (M+H)+(calculated: 508.04).

### Intermediate Compound 4:

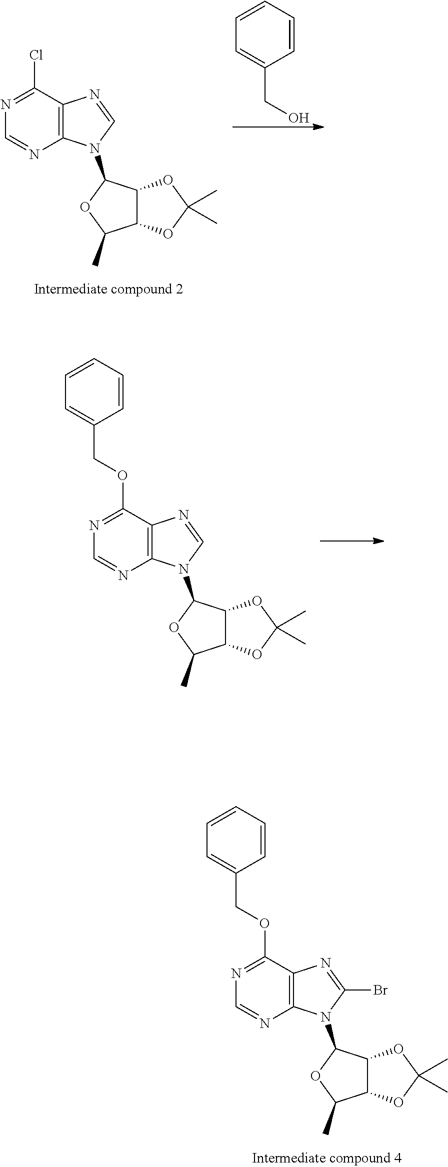 [SMILES: C[C@H]1O[C@@H](n2cnc3c(Cl)ncnc32)[C@@H]2OC(C)(C)O[C@@H]21 ; OCc1ccccc1 ; C[C@H]1O[C@@H](n2cnc3c(OCc4ccccc4)ncnc32)[C@@H]2OC(C)(C)O[C@@H]21 ; C[C@H]1O[C@@H](n2c(Br)nc3c(OCc4ccccc4)ncnc32)[C@@H]2OC(C)(C)O[C@@H]21]

Intermediate Compound 4 was prepared following protocols described for Intermediate Compound 3 using Intermediate Compound 2 and benzyl alcohol.

LCMS: Rt=0.673 min; (ESI positive ion) m/z: 461.2 (M+H)+(calculated: 461.07).

### Intermediate Compound 5:

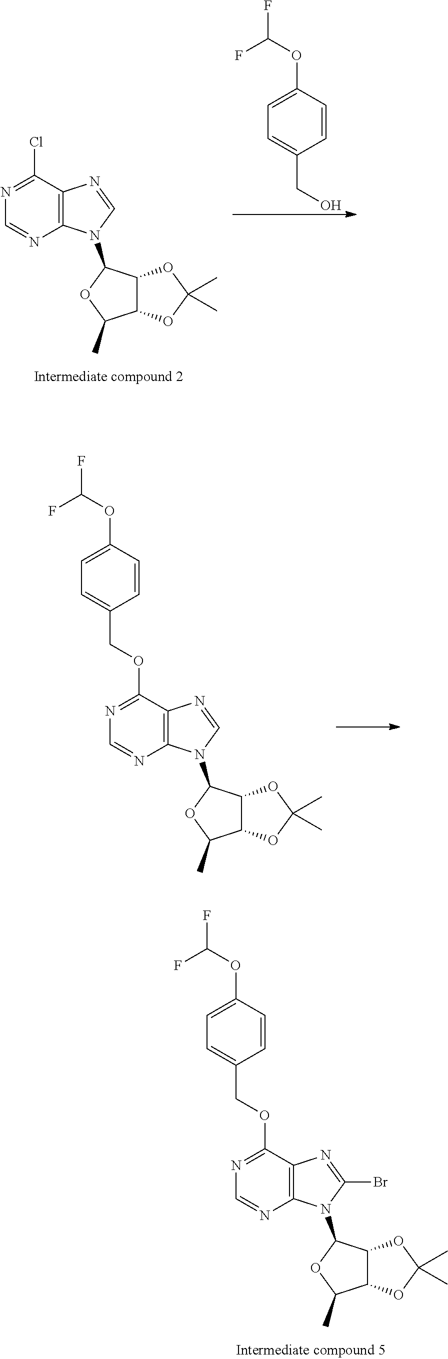

Intermediate Compound 5 was prepared following protocols described for Intermediate Compound 3 using Intermediate Compound 2 and (4-(difluoromethoxy)phenyl)methanol.

### Intermediate Compound 6:

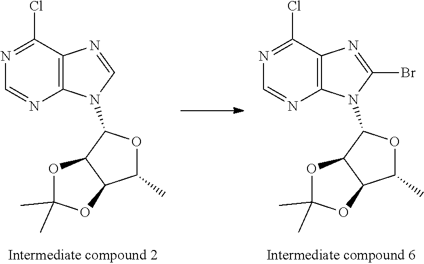 [SMILES: C[C@H]1O[C@@H](n2cnc3c(Cl)ncnc32)[C@@H]2OC(C)(C)O[C@@H]21 ; C[C@H]1O[C@@H](n2c(Br)nc3c(Cl)ncnc32)[C@@H]2OC(C)(C)O[C@@H]21]

To a solution of Intermediate Compound 2 (5 g, 16.09 mmol, 1 eq) in THE (80 mL) was added LDA (2 M, 24.14 mL, 3 eq) dropwise at −65° C. under N₂ atmosphere. The mixture was stirred at −65° C. for 45 min. After 45 min, a solution of 1,2-dibromo-1,1,2,2-tetrachloroethane (10.48 g, 32.18 mmol, 3.87 mL, 2 eq) in THE (20 mL) was dropwise added to the mixture. The mixture was stirred at −65° C. for 2 hr. The reaction mixture was then slowly poured into a saturated aqueous solution of NH₄Cl (200 mL) under ice-water bath cooling. The aqueous phase was extracted with EtOAc (100 mL×3) and the combined organic phases were washed with brine, dried over anhydrous Na₂SO₄ and concentrated to give a residue. The residue was purified by flash silica gel chromatography (ISCO®; 80 g SepaFlash® Silica Flash Column, Eluent of 0-17% Ethyl acetate/Petroleum ether gradient @100 mL/min) to afford Intermediate Compound 6 (2.95 g, 47% yield) as a yellow solid.

LCMS: Rt=0.519 min; (ESI positive ion) m/z: 390.9 (M+H)+(calculated: 390.99).

### Intermediate Compound 7:

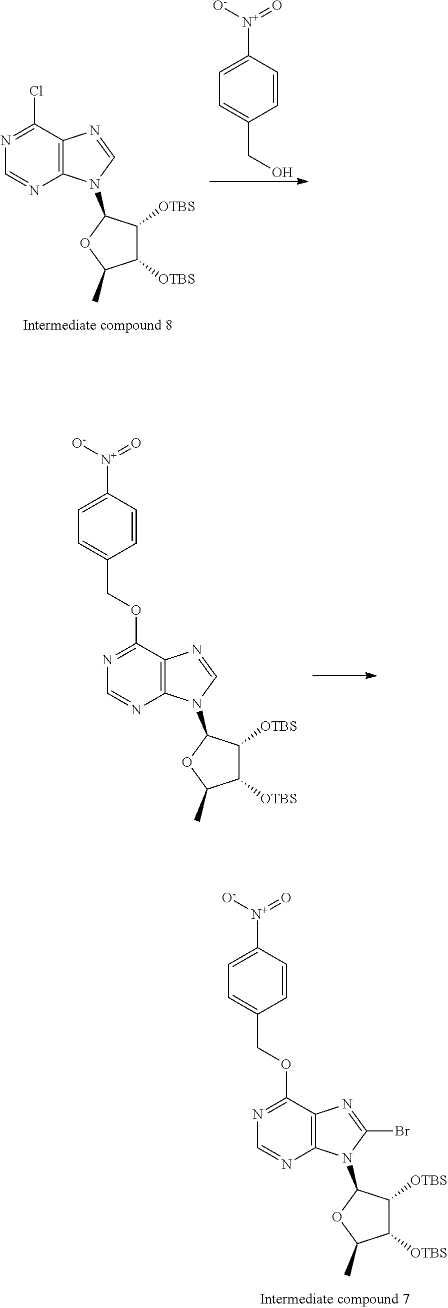 [SMILES: C[C@H]1O[C@@H](n2cnc3c(Cl)ncnc32)[C@H](O[Si](C)(C)C(C)(C)C)[C@@H]1O[Si](C)(C)C(C)(C)C ; O=[N+]([O-])c1ccc(CO)cc1 ; C[C@H]1O[C@@H](n2cnc3c(OCc4ccc([N+](=O)[O-])cc4)ncnc32)[C@H](O[Si](C)(C)C(C)(C)C)[C@@H]1O[Si](C)(C)C(C)(C)C ; C[C@H]1O[C@@H](n2c(Br)nc3c(OCc4ccc([N+](=O)[O-])cc4)ncnc32)[C@H](O[Si](C)(C)C(C)(C)C)[C@@H]1O[Si](C)(C)C(C)(C)C]

Intermediate Compound 7 was prepared following protocols described for Intermediate Compound 9 using Intermediate Compound 8 and (4-nitrophenyl)methanol.

LCMS: Rt=0.940 min; (ESI positive ion) m/z: 696.2 (M+H)+(calculated: 696.20).

### Intermediate Compound 8:

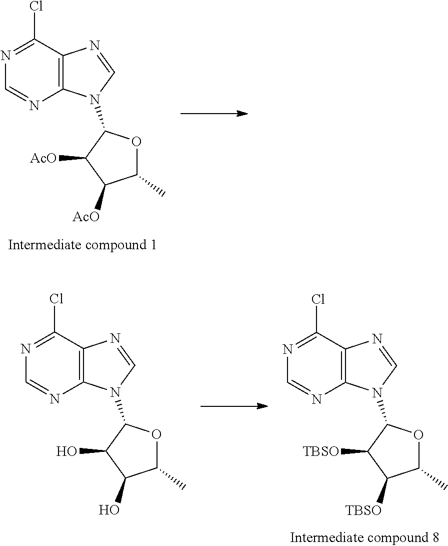 [SMILES: CC(=O)O[C@@H]1[C@H](OC(C)=O)[C@@H](C)O[C@H]1n1cnc2c(Cl)ncnc21 ; C[C@H]1O[C@@H](n2cnc3c(Cl)ncnc32)[C@H](O)[C@@H]1O ; C[C@H]1O[C@@H](n2cnc3c(Cl)ncnc32)[C@H](O[Si](C)(C)C(C)(C)C)[C@@H]1O[Si](C)(C)C(C)(C)C]

To a solution of Intermediate Compound 1 (59 g, 153.01 mmol, 92% purity, 1 eq) in MeOH (300 mL) was added NH₃·H₂O (45.50 g, 363.52 mmol, 50 mL, 28% purity, 2.38 eq) at −40° C. The mixture was stirred at 0° C. for 0.5 hr. The reaction mixture was concentrated under reduced pressure to afford the corresponding diol (41 g, crude) as a white solid, which was used directly for the next step.

LCMS: Rt=0.604 min; (ESI positive ion) m/z: 271.00 (M+H)+(calculated: 271.05).

To a solution of the diol compound (41 g, 151.47 mmol, 1 eq) in DCM (300 mL) was added one drop DMF at 0° C., DIEA (391.54 g, 3.03 mol, 527.68 mL, 20 eq) and TBSOTf (400.40 g, 1.51 mol, 348.18 mL, 10 eq) were added to the mixture at 0° C. The mixture was stirred at 20° C. for 16 hr. The reaction mixture was poured into a cold solution of 1 N HCl (1000 mL) and stirred for 1 min, and then extracted with DCM (250 mL×2). The combined organic layers were dried with anhydrous Na₂SO₄, filtered and concentrated under reduced pressure to give a residue. The residue was purified by flash silica gel chromatography (ISCO®; 330 g SepaFlash® Silica Flash Column, Eluent of 0-28% Ethyl acetate/Petroleum ether gradient @100 mL/min) to afford Intermediate Compound 8 (66 g, 85% yield) as a white solid.

LCMS: Rt=0.607 min; (ESI positive ion) m/z: 499.10 (M+H)+(calculated: 499.22).

### Intermediate Compound 9:

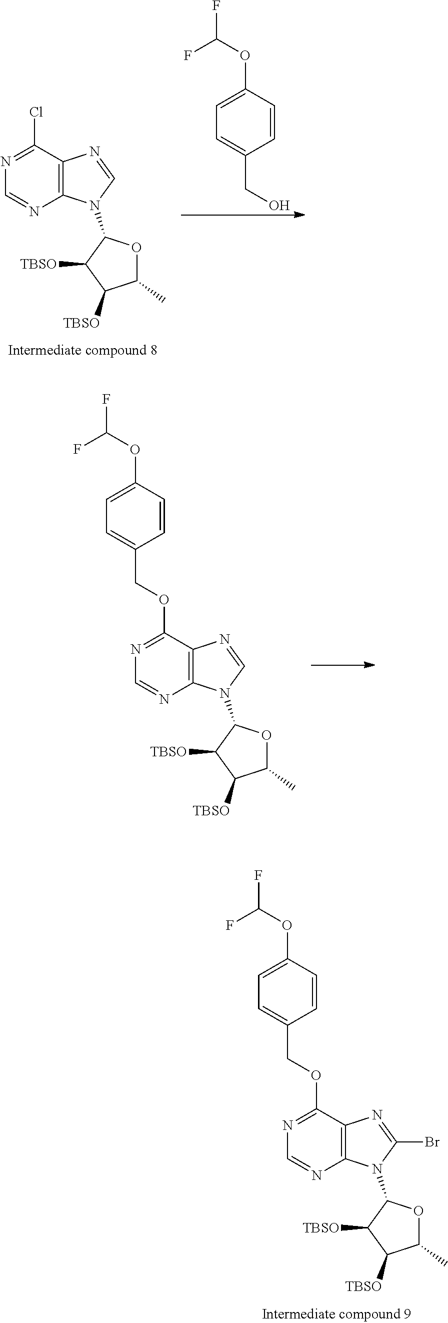 [SMILES: C[C@H]1O[C@@H](n2cnc3c(Cl)ncnc32)[C@H](O[Si](C)(C)C(C)(C)C)[C@@H]1O[Si](C)(C)C(C)(C)C ; OCc1ccc(OC(F)F)cc1 ; C[C@H]1O[C@@H](n2cnc3c(OCc4ccc(OC(F)F)cc4)ncnc32)[C@H](O[Si](C)(C)C(C)(C)C)[C@@H]1O[Si](C)(C)C(C)(C)C ; C[C@H]1O[C@@H](n2c(Br)nc3c(OCc4ccc(OC(F)F)cc4)ncnc32)[C@H](O[Si](C)(C)C(C)(C)C)[C@@H]1O[Si](C)(C)C(C)(C)C]

To a mixture of Intermediate Compound 8 (4 g, 8.01 mmol, 1 eq), (4-(difluoromethoxy)phenyl)methanol (1.67 g, 9.62 mmol, 1.2 eq), Cs2CO₃ (6.53 g, 20.03 mmol, 2.5 eq), and Xantphos (927.29 mg, 1.60 mmol, 0.2 eq) in toluene (40 mL) was added Pd2(dba)₃ (733.76 mg, 801.29 μmol, 0.1 eq). The mixture was stirred at 80° C. for 4 hr. The reaction mixture was then concentrated under reduced pressure to give a residue, which was purified by Prep-TLC (SiO₂, Petroleum ether:Ethyl acetate=5:1) to afford the corresponding diol (4.1 g, 73% yield) as a red solid.

LCMS: Rt=0.844 min; (ESI positive ion) m/z: 637.20 (M+H)+(calculated: 637.30).

To a solution of the diol compound (500 mg, 785.08 μmol, 1 eq) in THE (5 mL) was added LDA (2 M, 1.96 mL, 5 eq) at −70° C. The mixture was stirred at −70° C. for 0.5 hr. A solution of carbon tetrabromide (1.04 g, 3.14 mmol, 4 eq) in THE (3 mL) was added to the mixture and the mixture was stirred at 25° C. for 12 hr under N₂ atmosphere. The mixture was poured into a cold saturated solution of NH₄Cl (100 mL) and stirred for 1 min. The aqueous phase was extracted with ethyl acetate (100 mL×3). The combined organic phase was washed with brine (100 mL), dried with anhydrous Na₂SO₄, filtered and concentrated under vacuum to give a residue. The residue was purified by flash silica gel chromatography (ISCO®; 4 g SepaFlash® Silica Flash Column, Eluent of 0-18% Ethyl acetate/Petroleum ether gradient @18 mL/min) to afford Intermediate Compound 9 (120 mg, 17% yield) as a yellow oil.

LCMS: Rt=0.875 min; (ESI positive ion) m/z: 717.40 (M+H)+(calculated: 717.21).

### Intermediate Compound 10:

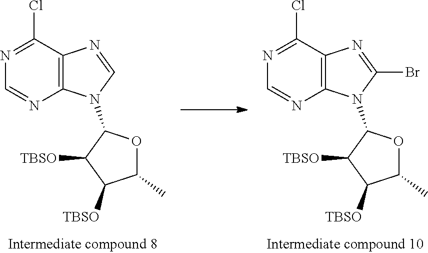 [SMILES: C[C@H]1O[C@@H](n2cnc3c(Cl)ncnc32)[C@H](O[Si](C)(C)C(C)(C)C)[C@@H]1O[Si](C)(C)C(C)(C)C ; C[C@H]1O[C@@H](n2c(Br)nc3c(Cl)ncnc32)[C@H](O[Si](C)(C)C(C)(C)C)[C@@H]1O[Si](C)(C)C(C)(C)C]

To a solution of Intermediate Compound 8 (86 g, 168.83 mmol, 98% purity, 1 eq) in THE (600 mL) was added LDA (2 M, 253.25 mL, 3 eq) at −65° C. The mixture was stirred at −65° C. for 0.5 hr. A solution of DBTCE (109.96 g, 337.67 mmol, 40.57 mL, 2 eq) in THE (200 mL) was added to the mixture and the mixture was stirred at −65° C. for 1.5 hr. The mixture was poured into cold saturated solution of NH₄Cl (600 mL). The aqueous phase was extracted with ethyl acetate (600 mL×2). The combined organic phase was washed with brine (200 mL), dried with anhydrous Na₂SO₄, filtered and concentrated under vacuum to give a residue. The residue was purified by flash silica gel chromatography (ISCO®; 330 g SepaFlash® Silica Flash Column, Eluent of 0-12% Ethyl acetate/Petroleum ether gradient @100 mL/min) to afford Intermediate Compound 10 (51 g, 48.07% yield) as a yellow oil.

LCMS: Rt=0.541 min; (ESI positive ion) m/z: 577.30/579.10 (M+H)+(calculated: 577.14/579.13).

1H NMR (400 MHz, CDCl₃-d) δ=8.72 (s, 1H), 6.03 (d, J=5.0 Hz, 1H), 5.32 (t, J=4.8 Hz, 1H), 4.34 (t, J=4.2 Hz, 1H), 4.23-4.16 (m, 1H), 1.43 (d, J=6.6 Hz, 3H), 0.98 (s, 9H), 0.82 (s, 9H), 0.16 (d, J=1.8 Hz, 6H), −0.04 (s, 3H), −0.33 (s, 3H).

### Intermediate Compound 11:

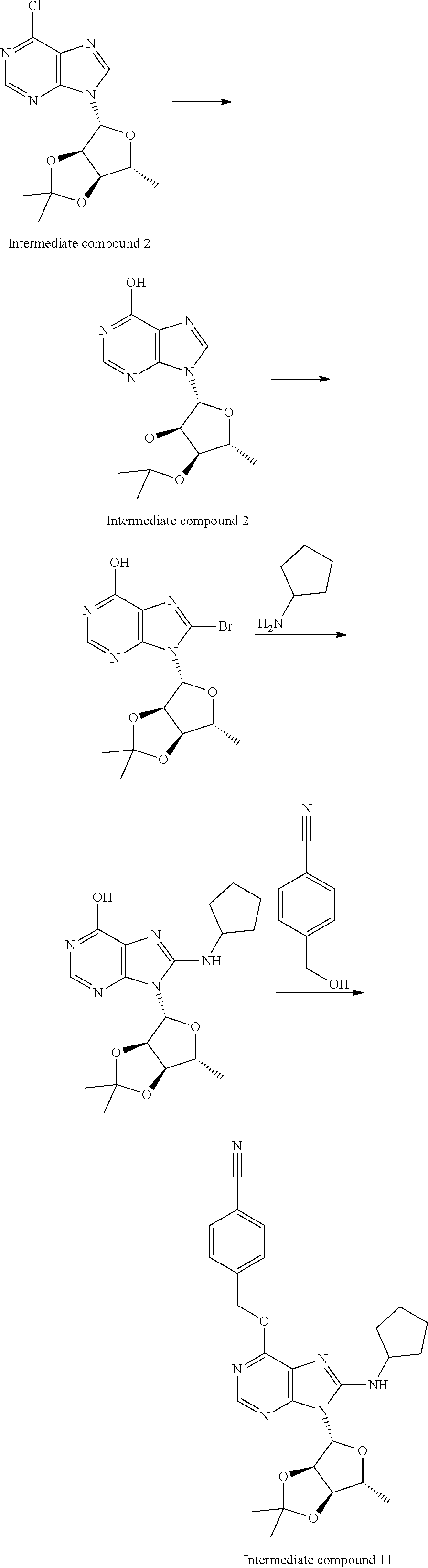

To a solution of Intermediate Compound 2 (2 g, 6.44 mmol, 1 eq) in dioxane (30 mL) and water (30 mL) were added DABCO (794.18 mg, 7.08 mmol, 778.61 μL, 1.1 eq) and K₂CO₃ (1.78 g, 12.87 mmol, 2 eq). The mixture was stirred at 90° C. for 0.5 hr. The dioxane was removed under reduced pressure and the aqueous phase was lyophilized to give a white solid. The white solid was washed with water (15 mL×3) and dried to afford the corresponding hydroxylated compound (1.87 g, 6.40 mmol, 99.40% yield) as a white solid.

LCMS: Rt=0.334 min; (ESI positive ion) m/z: 293.00 (M+H)+(calculated: 293.12).

1H NMR (400 MHz, DMSO-d6) δ=8.27 (s, 1H), 8.08 (s, 1H), 6.05 (d, J=2.6 Hz, 1H), 5.40 (dd, J=2.6, 6.4 Hz, 1H), 4.76 (dd, J=3.4, 6.2 Hz, 1H), 4.24 (dq, J=3.6, 6.6 Hz, 1H), 1.52 (s, 3H), 1.31 (s, 3H), 1.26 (d, J=6.6 Hz, 3H).

To a 10% aqueous solution of Na₂HPO₄ (45 mL) at 20° C. was added bromine (7.75 g, 48.50 mmol, 2.5 mL, 22.50 eq) and the mixture was stirred vigorously for 15 min until most of the bromine had dissolved. The decanted bromine solution was added to a solution of the preceding hydroxylated compound (630 mg, 2.16 mmol, 1 eq) in dioxane (20 mL). The mixture was stirred for 72 hr at 20° C. After cooling in an ice/water bath, an aqueous solution of NaHSO₃ (2 N) was added dropwise until the solution became colorless. The water layer was extracted with DCM (30 mL×3). The organic layer was washed with an aqueous NaHSO₃ solution (0.2 N, 50 mL) and water (50 mL), dried over Na₂SO₄, filtered and concentrated under vacuum to give a residue. The residue was purified by column chromatography (SiO₂, Petroleum ether/Ethyl acetate=1/0 to 0/1) to afford the corresponding brominated compound (200 mg, 25% yield) as a white solid.

LCMS: Rt=0.398 min; (ESI positive ion) m/z: 370.90 (M+H)+(calculated: 371.03).

To a solution of the preceding brominated compound (200 mg, 538.81 μmol, 1 eq) in dioxane (4 mL) was added cyclopentanamine (688.18 mg, 8.08 mmol, 797.42 μL, 15 eq). The mixture was stirred at 100° C. for 36 hr. The mixture was concentrated to give a residue. The residue was purified by Prep-TLC (SiO₂, EtOAc:MeOH=20:1) to afford the corresponding amine (170 mg, 84% yield) as a brown solid.

LCMS: Rt=0.407 min; (ESI positive ion) m/z: 376.10 (M+H)+(calculated: 376.19).

To a mixture of the preceding amine compound (120 mg, 319.64 μmol, 1 eq), 4-(hydroxymethyl)benzonitrile (63.84 mg, 479.46 μmol, 1.5 eq) and triphenylphosphine (251.51 mg, 958.92 μmol, 3 eq) in dioxane (2 mL) was added DEAD (167.00 mg, 958.92 μmol, 174.32 μL, 3 eq) dropwise under N₂ atmosphere. The mixture was stirred at 20° C. for 16 hr. The reaction mixture was filtered. The filtrate was purified by Prep-TLC (SiO₂, PE:EtOAc=2:1) to afford Intermediate Compound 11 (48 mg, 30% yield) as a white solid.

LCMS: Rt=0.586 min; (ESI positive ion) m/z: 491.10 (M+H)+(calculated: 491.23).

### Intermediate Compound 12:

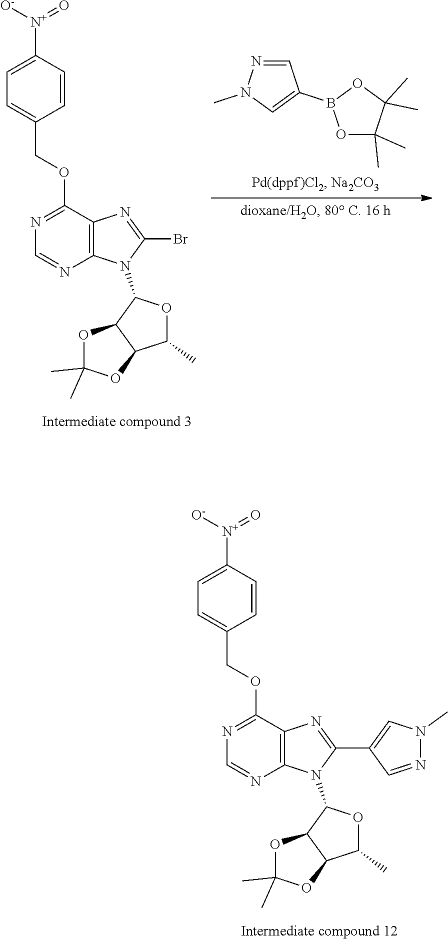

To a mixture of Intermediate Compound 3 (160 mg, 316.01 μmol, 1 eq), 1-methyl-4-(4,4,5,5-tetramethyl-1,3,2-dioxaborolan-2-yl)-1H-pyrazole (65.75 mg, 316.01 μmol, 1 eq) and Na₂CO₃ (100.48 mg, 948.04 μmol, 3 eq) in dioxane (4 mL) and water (0.4 mL) was added Pd(dppf)Cl₂·CH₂Cl₂ (25.81 mg, 31.60 μmol, 0.1 eq) under N₂ atmosphere. The mixture was stirred at 80° C. for 16 hr under N₂ atmosphere. The mixture was filtered and concentrated under vacuum to give a residue. The residue was purified by prep-TLC (Petroleum ether/Ethyl acetate=1/1) to afford Intermediate Compound 12 (63 mg, 37% yield) as a yellow oil.

LCMS: Rt=0.603 min; (ESI positive ion) m/z: 508.10 (M+H)+(calculated: 508.19).

### Intermediate Compound 13:

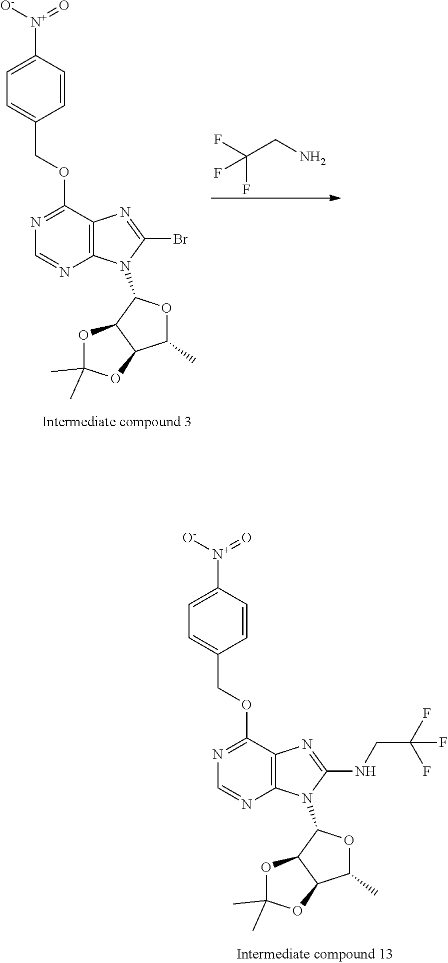 [SMILES: C[C@H]1O[C@@H](n2c(Br)nc3c(OCc4ccc([N+](=O)[O-])cc4)ncnc32)[C@@H]2OC(C)(C)O[C@@H]21 ; NCC(F)(F)F ; C[C@H]1O[C@@H](n2c(NCC(F)(F)F)nc3c(OCc4ccc([N+](=O)[O-])cc4)ncnc32)[C@@H]2OC(C)(C)O[C@@H]21]

To a solution of Intermediate Compound 3 (80 mg, 158.01 μmol, 1 eq) in toluene (10 mL) was added 2,2,2-trifluoroethan-1-amine (78.26 mg, 790.04 μmol, 62.11 μL, 5 eq), Pd₂(dba)₃ (14.47 mg, 15.80 μmol, 0.1 eq), Xantphos (15.06 mg, 31.60 μmol, 0.2 eq) and Cs₂CO₃ (102.96 mg, 316.01 μmol, mol, 2 eq). The solution was degassed and purged with N₂ 3 times, and then stirred at 110° C. for 2 hr under N₂ atmosphere. The mixture was concentrated under vacuum to give a residue. The residue was purified by prep-TLC (SiO₂, Petroleum ether:Ethyl acetate=2:1) to afford Intermediate Compound 13 (20 mg, 24% yield) as a white solid.

LCMS: Rt=0.611 min; (ESI positive ion) m/z: 525.50 (M+H)+(calculated: 525.16).

### Intermediate Compound 14:

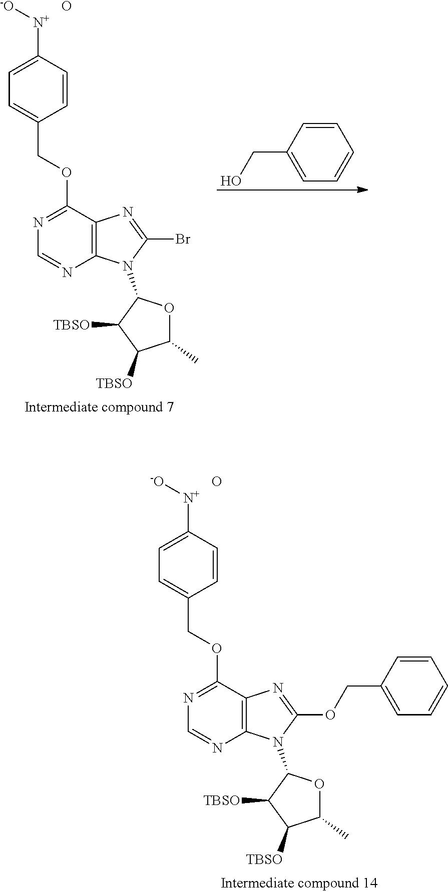

A mixture of Intermediate Compound 7 (48 mg, 69.09 μmol, 1 eq), benzyl alcohol (11.21 mg, 103.63 μmol, 10.78 μL, 1.5 eq), Pd₂(dba)₃ (6.33 mg, 6.91 μmol, 0.1 eq), Xantphos (8.00 mg, 13.82 μmol, 0.2 eq) and CS2CO₃ (56.28 mg, 172.72 μmol, 2.5 eq) in toluene (2 mL) was degassed and purged with N₂ for 3 times, and then stirred at 80° C. for 1 hr under N₂ atmosphere. The reaction mixture was concentrated under reduced pressure to give a residue. The residue was purified by Prep-TLC (SiO₂, Petroleum ether:Ethyl acetate=3:1) to afford Intermediate Compound 14 (12 mg, 24% yield) as a yellow oil.

LCMS: Rt=0.901 min; (ESI positive ion) m/z: 722.30 (M+H)+(calculated: 722.33).

### Intermediate Compound 15:

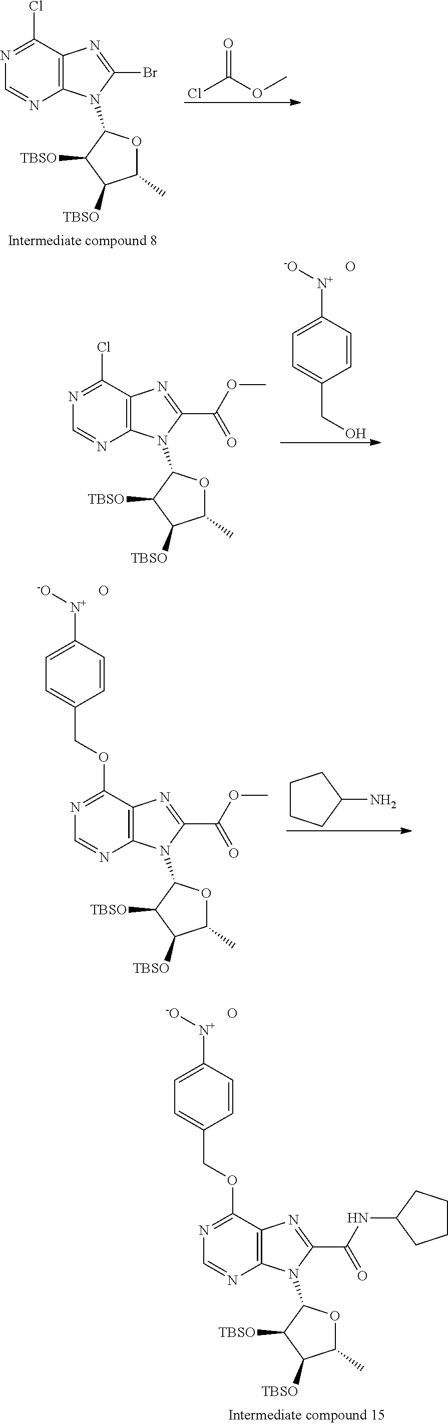

To a solution of Intermediate Compound 8 (500 mg, 1.00 mmol, 1 eq) in THF (8 mL) was added LDA (2 M, 751.21 μL, 1.5 eq) dropwise at −65° C. under N₂ atmosphere. The mixture was stirred at −65° C. for 45 min. After 45 min, a solution of methyl chloroformate (189.30 mg, 2.00 mmol, 155.16 μL, 2 eq) in THF (2 mL) was added dropwise to the mixture. The mixture was slowly warmed to 20° C. and stirred for 12 hr. The reaction mixture was slowly poured into a saturated aqueous solution of NH₄Cl (30 mL) under ice-water bath cooling. The aqueous phase was extracted with EtOAc (20 mL×3) and the combined organic phases were washed with brine, dried over anhydrous Na₂SO₄ and concentrated to give a residue. The residue was purified by flash silica gel chromatography (ISCO®; 20 g SepaFlash® Silica Flash Column, Eluent of 0-10% Ethyl acetate/Petroleum ether gradient @80 mL/min) to afford corresponding ester compound (340 mg, 61% yield) as a yellow oil.

LCMS: Rt=0.647 min; (ESI positive ion) m/z: 557.10 (M+H)+(calculated: 557.23).

To a solution of the preceding ester compound (290 mg, 520.43 μmol, 1 eq) in toluene (4 mL) were added (4-nitrophenyl)methanol (119.54 mg, 780.65 μmol, 1.5 eq), Cs₂CO₃ (423.92 mg, 1.30 mmol, 2.5 eq), Xantphos (60.23 mg, 104.09 μmol, 0.2 eq) and Pd₂(dba)₃ (47.66 mg, 52.04 μmol, 0.1 eq). The mixture was stirred at 80° C. for 1 hr under N₂ atmosphere. The reaction mixture was then diluted with EtOAc (20 mL) and filtered. The filter cake was washed with EtOAc (10 mL×3). The filtrate was concentrated under reduced pressure to give a residue. The residue was purified by Prep-TLC (SiO₂, PE:EtOAc=3:1) to afford the corresponding ether compound (267 mg, 76% yield) as a red oil.

LCMS: Rt=0.676 min; (ESI positive ion) m/z: 674.20 (M+H)+(calculated: 674.30).

A solution of the preceding ether compound (210 mg, 311.62 μmol, 1 eq) in cyclopentanamine (1.73 g, 20.27 mmol, 2 mL, 65.05 eq) was stirred at 20° C. for 1 hr. The reaction mixture was diluted by water (20 mL) and extracted with EtOAc (20 mL×3). The organic layer was washed with brine, and dried with Na₂SO₄. The solution was concentrated to give a residue. The residue was purified by Prep-TLC (SiO₂, PE:EtOAc=3:1) to afford Intermediate Compound 15 (65 mg, 29% yield) as a yellow gum.

LCMS: Rt=0.660 min; (ESI positive ion) m/z: 727.30 (M+H)+(calculated: 727.36).

### Intermediate Compound 16:

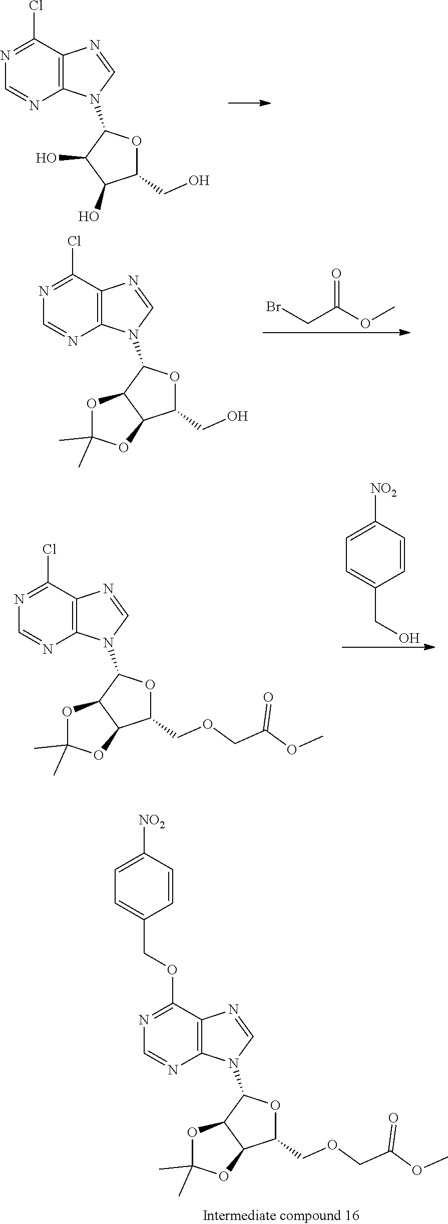

A mixture of (2R,3R,4S,5R)-2-(6-chloro-9H-purin-9-yl)-5-(hydroxymethyl)tetrahydrofuran-3,4-diol (9 g, 31.39 mmol, 1 eq) and TsOH·H₂O (59.72 g, 313.95 mmol, 10 eq) in acetone (200 mL) was degassed and purged with N₂ 3 times. The mixture was stirred at 25° C. for 3 hr under N₂ atmosphere. The mixture was filtered and concentrated under reduced pressure to give a residue, which was purified by column chromatography (SiO₂, Petroleum ether/Ethyl acetate=100/1 to 1/1) to afford the corresponding acetonide (7.16 g, 61% yield) as a white solid.

1H NMR (400 MHz, CDCl₃) δ=8.78-8.74 (m, 1H), 8.28 (br d, J=1.4 Hz, 1H), 6.00 (br d, J=4.4 Hz, 1H), 5.21-5.10 (m, 2H), 4.96 (br d, J=4.6 Hz, 1H), 4.55 (br s, 1H), 3.97 (br d, J=12.6 Hz, 1H), 3.82 (br d, J=12.4 Hz, 1H), 1.64 (br s, 3H), 1.38 (br s, 3H).

To a mixture of the preceding compound (7.1 g, 21.73 mmol, 1 eq) and methyl 2-bromoacetate in THE (80 mL) was added sodium hydride (1.30 g, 32.60 mmol, 60% purity, 1.5 eq) at 0° C. The mixture was stirred at 0° C. for 1 hr. The mixture was then poured into a cold solution of HCl (0.5 N 800 mL) and stirred for 1 min. The aqueous phase was extracted with ethyl acetate (300 mL×2). The combined organic phase was washed with brine (800 mL), dried with anhydrous Na₂SO₄, filtered and concentrated under vacuum to give a residue. The residue was purified by column chromatography (SiO₂, Petroleum ether/Ethyl acetate=100/1 to 1/4) to afford the corresponding ester (7.7 g, 89% yield) as a yellow oil.

LCMS: Rt=0.466 min; (ESI positive ion) m/z: 399.00 (M+H)+(calculated: 399.10).

A mixture of the preceding compound (7.7 g, 19.31 mmol, 1 eq), (4-nitrophenyl)methanol (3.55 g, 23.17 mmol, 1.2 eq), Pd₂(dba)₃ (1.77 g, 1.93 mmol, 0.1 eq), Xantphos (2.23 g, 3.86 mmol, 0.2 eq) and Cs₂CO₃ (15.73 g, 48.27 mmol, 2.5 eq) in toluene (80 mL) was degassed and purged with N₂ 3 times. The mixture was stirred at 80° C. for 0.5 hr under N2 atmosphere. The mixture was filtered and concentrated under vacuum. The resultant residue was purified by column chromatography (SiO₂, Petroleum ether/Ethyl acetate=100/1 to 1/1) to afford Intermediate Compound 16 (4 g, 40% yield) as a yellow oil.

### Intermediate Compound 17:

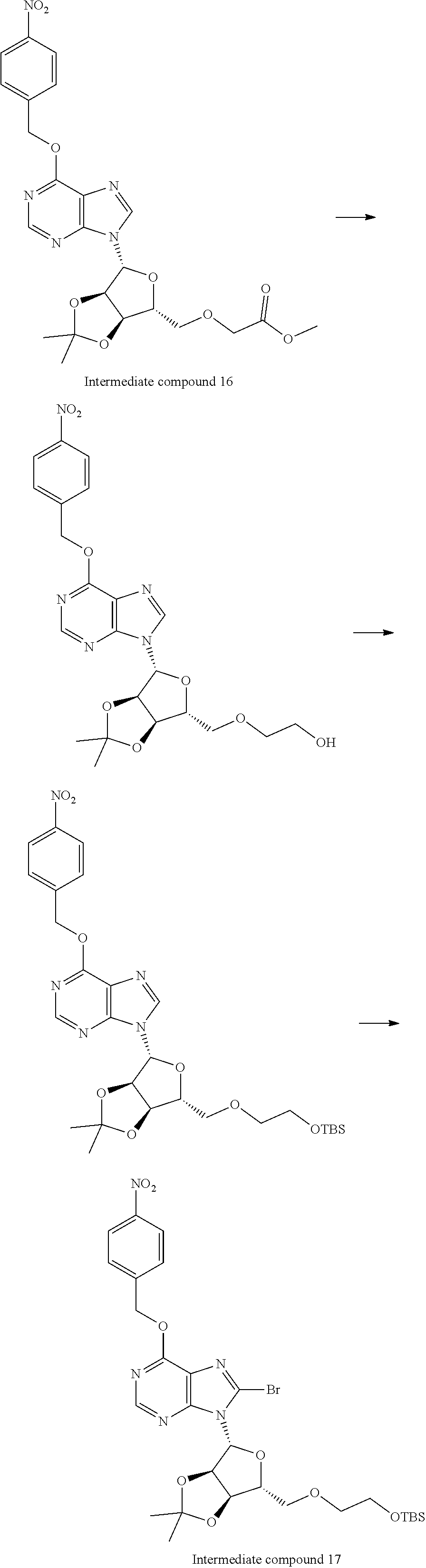 [SMILES: COC(=O)COC[C@H]1O[C@@H](n2cnc3c(OCc4ccc([N+](=O)[O-])cc4)ncnc32)[C@@H]2OC(C)(C)O[C@@H]21 ; CC1(C)O[C@@H]2[C@H](O1)[C@@H](COCCO)O[C@H]2n1cnc2c(OCc3ccc([N+](=O)[O-])cc3)ncnc21 ; CC1(C)O[C@@H]2[C@H](O1)[C@@H](COCCO[Si](C)(C)C(C)(C)C)O[C@H]2n1cnc2c(OCc3ccc([N+](=O)[O-])cc3)ncnc21 ; CC1(C)O[C@@H]2[C@H](O1)[C@@H](COCCO[Si](C)(C)C(C)(C)C)O[C@H]2n1c(Br)nc2c(OCc3ccc([N+](=O)[O-])cc3)ncnc21]

To a solution of Intermediate Compound 16 (500 mg, 969.99 μmol, 1 eq) in THE (5 mL) was added LiBH₄ (2 M, 533.49 μL, 1.1 eq) at 0° C. under N₂. The mixture was stirred at 0° C. for 1 hr and then the mixture was added to water (100 mL) dropwise at 0° C. and stirred for 5 min. The mixture was then extracted with EtOAc (50 mL×2). The combined organic phase was washed with brine (100 mL), dried with anhydrous Na₂SO₄, filtered and concentrated under vacuum to give a residue. The residue was purified by Prep-TLC (Petroleum ether/Ethyl acetate=0/1) to afford the corresponding alcohol (250 mg, 53% yield) as a yellow oil.

LCMS: Rt=0.399 min; (ESI positive ion) m/z: 488.1 (M+H)+(calculated: 488.17).

To a mixture of the preceding alcohol compound (300 mg, 615.43 μmol, 1 eq) and imidazole (83.79 mg, 1.23 mmol, 2 eq) in DCM (10 mL) was added TBSCI (139.14 mg, 923.15 mol, 113.12 μL, 1.5 eq) at 0° C. The mixture was stirred at 20° C. for 16 hr. The mixture was then concentrated under vacuum to give a residue. The residue was purified by column chromatography (SiO2, Petroleum ether/Ethyl acetate=100/1 to 3/1) to afford the corresponding t-butyldimethylsilyl ether (300 mg, 493.58 μmol, 80.20% yield, 99% purity) as a yellow oil. LCMS: Rt=0.723 min; (ESI positive ion) m/z: 602.1 (M+H)+(calculated: 602.26).

To a solution of the preceding compound (240 mg, 398.86 μmol, 1 eq) in THF (3 mL) was added LDA (2 M, 997.14 μL, 5 eq) at −70° C. The mixture was stirred at −70° C. for 0.5 hr. A solution of CBr₄ (529.08 mg, 1.60 mmol, 4 eq) in THE (3 mL) was added to the mixture, and the mixture was stirred at 25° C. for 2.5 hr. The mixture was poured into a cold saturated solution of NH₄Cl (40 mL) and stirred for 1 min. The aqueous phase was extracted with ethyl acetate (20 mL×2). The combined organic phase was washed with brine (40 mL), dried with anhydrous Na₂SO₄, filtered and concentrated under vacuum to give a residue. The residue was purified by Prep-TLC (Petroleum ether/Ethyl acetate=3/1) to afford Intermediate Compound 17 (120 mg, 42% yield) as a yellow oil.

LCMS: Rt=0.746 min; (ESI positive ion) m/z: 682.1 (M+H)+(calculated: 682.17).

### Intermediate Compound 18:

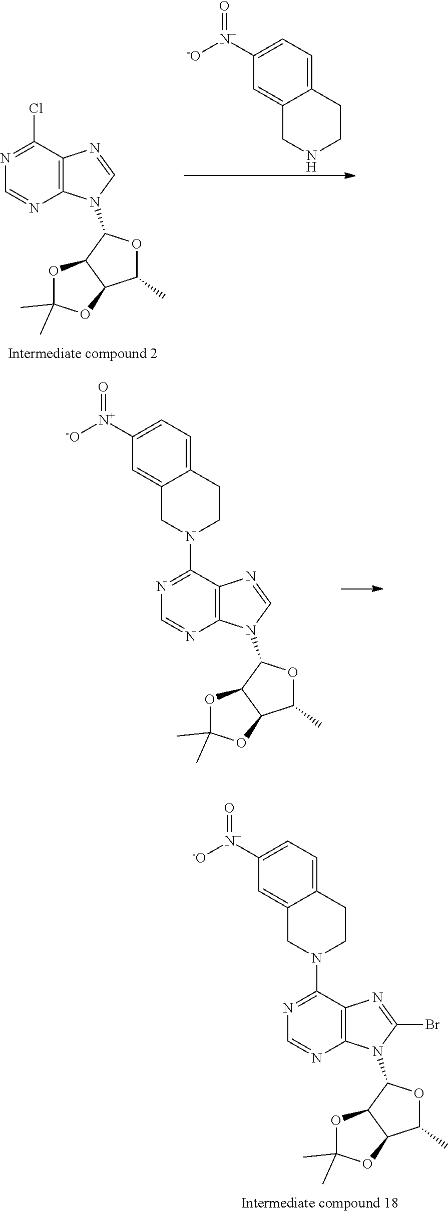

A mixture of Intermediate Compound 2 (600 mg, 1.93 mmol, 1 eq), 7-nitro-1,2,3,4-tetrahydroisoquinoline (516.09 mg, 2.90 mmol, 1.5 eq) and TEA (1.56 g, 15.45 mmol, 2.15 mL, 8 eq) in EtOH (10 mL) was stirred at 80° C. for 1 hr. The mixture was concentrated under vacuum to give a residue. The residue was purified by column chromatography (SiO2, Petroleum ether/Ethyl acetate=100/1 to 3/1) to afford the corresponding amine compound (700 mg, 73% yield) as a yellow oil.

LCMS: Rt=0.451 min; (ESI positive ion) m/z: 453.2 (M+H)+(calculated: 453.18).

To a solution of the above amine compound (100 mg, 221.01 μmol, 1 eq) in THE (5 mL) was added LDA (2 M, 552.53 μL, 5 eq) at −65° C. The mixture was stirred at −65° C. for 0.5 hr. A solution of carbon tetrabromide (293.18 mg, 884.05 μmol, 4 eq) in THE (5 mL) was added to the mixture, and the mixture was stirred at 20° C. for 11.5 hr. The mixture was poured into ice-cold saturated solution of NH4Cl (20 mL) and stirred for 1 min. The aqueous phase was extracted with ethyl acetate (10 mL×2). The combined organic phase was washed with brine (20 mL), dried with anhydrous Na2SO4, filtered and concentrated under vacuum to give a residue. The residue was purified by column chromatography (SiO2, Petroleum ether/Ethyl acetate=100/1 to 3/1) to afford Intermediate Compound 18 (37 mg, 30% yield) as a yellow oil.

LCMS: Rt=0.588 min; (ESI positive ion) m/z: 533.0 (M+H)+(calculated: 533.09).

### Intermediate Compound 19:

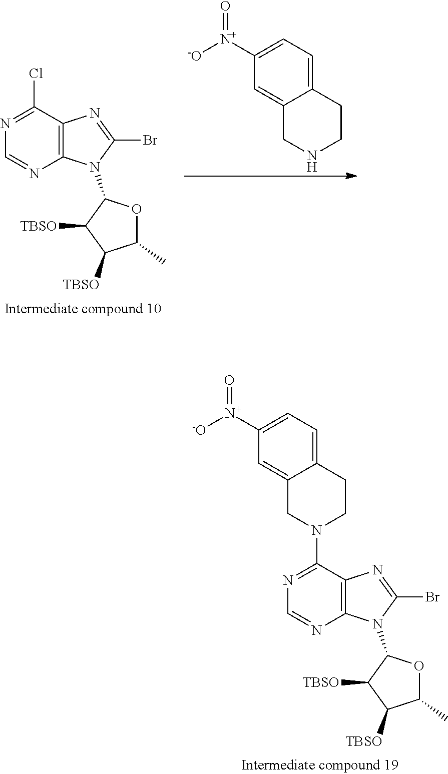

To a solution of Intermediate Compound 10 (2 g, 3.04 mmol, 1 eq) in EtOH (10 mL) was added 7-nitro-1,2,3,4-tetrahydroisoquinoline (542.49 mg, 3.04 mmol, 1 eq) and TEA (924.21 mg, 9.13 mmol, 1.27 mL, 3 eq). The mixture was stirred at 80° C. for 1 hr and then concentrated under vacuum to give a residue. The residue was purified by column chromatography (SiO2, Petroleum ether/Ethyl acetate=100/1 to 1/1), followed by TLC (Petroleum ether/Ethyl acetate=5/1) afford Intermediate Compound 19 (390 mg, 18% yield) as a yellow oil.

LCMS: Rt=0.717 min; (ESI positive ion) m/z: 721.0 (M+H)+(calculated: 721.23).

### Intermediate Compound 20:

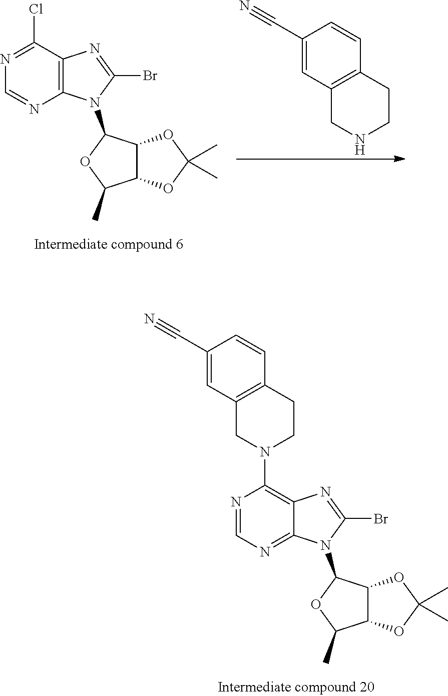 [SMILES: C[C@H]1O[C@@H](n2c(Br)nc3c(Cl)ncnc32)[C@@H]2OC(C)(C)O[C@@H]21 ; N#Cc1ccc2c(c1)CNCC2 ; C[C@H]1O[C@@H](n2c(Br)nc3c(N4CCc5ccc(C#N)cc5C4)ncnc32)[C@@H]2OC(C)(C)O[C@@H]21]

To a solution of Intermediate Compound 6 (120 mg, 307.98 μmol, 1 eq) in ethanol (5 mL) was added 1,2,3,4-tetrahydroisoquinoline-7-carbonitrile (48.72 mg, 307.98 μmol, 1 eq) and TEA (249.32 mg, 2.46 mmol, 342.94 μL, 8 eq). The mixture was stirred at 80° C. for 12 hr and then concentrated under vacuum to give a residue. The residue was purified by Prep-TLC (SiO2, Petroleum ether:Ethyl acetate=3:1) to afford Intermediate Compound 20 (82 mg, 50% yield) as a white solid.

LCMS: Rt=0.606 min; (ESI positive ion) m/z: 511.1 (M+H)+(calculated: 511.10).

### Intermediate Compound 21:

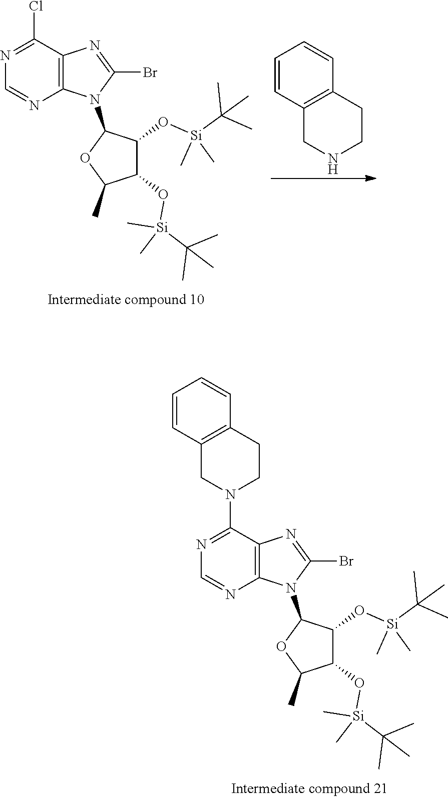

To a solution of Intermediate Compound 10 (2 g, 3.46 mmol, 1 eq in EtOH (10 mL) was added TEA (1.05 g, 10.38 mmol, 1.44 mL, 3 eq and 1,2,3,4-tetrahydroisoquinoline (460.79 mg, 3.46 mmol, 434.71 μL, 1 eq. The mixture was stirred at 80° C. for 1 hr. The reaction mixture was concentrated under reduced pressure to give a residue. The residue was purified by flash silica gel chromatography (ISCO®; 20 g SepaFlash® Silica Flash Column, Eluent of 0-11% Ethyl acetate/Petroleum ether gradient @80 mL/min) to afford Intermediate Compound 21 (1.4 g, 60% yield) as a yellow oil.

LCMS: Rt=0.677 min; (ESI positive ion) m/z: 674.5 (M+H)+(calculated: 674.25).

### Intermediate Compound 22:

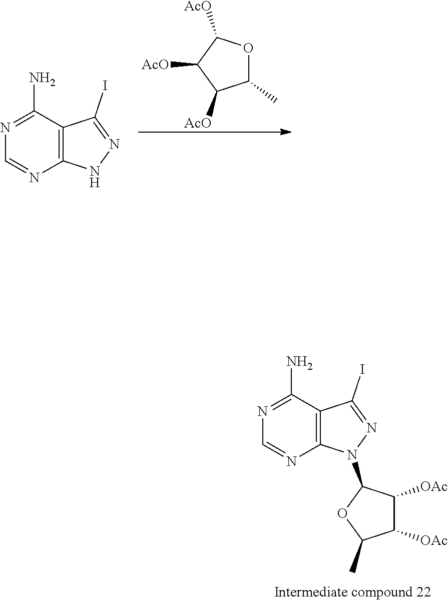

To a solution of 3-iodo-1H-pyrazolo[3,4-d]pyrimidin-4-amine (500 mg, 1.92 mmol, 1) in MeCN (5 mL) was added BSA (1.56 g, 7.66 mmol, 1.89 mL, 4 q). The mixture was stirred at 50° C. for 2 hr under N2 atmosphere. After 2 hr, the mixture was concentrated under vacuum to give a residue. The residue and (2S,3R,4R,5R)-5-methyltetrahydrofuran-2,3,4-triyl triacetate (548.35 mg, 2.11 mmol, 1.1 eq) were dissolved in MeCN (5 mL) and cooled to 1° C. Then TMSOTf (1.28 g, 5.75 mmol, 1.04 mL, 3) was added dropwise to the mixture at 0° C. The mixture was slowly warmed to 20° C. for 12 hr under N2 atmosphere. The reaction mixture was poured into aqueous NaHCO3 (50 mL) under ice-water bath cooling and stirred for 3 min. The aqueous phase was extracted with ethyl acetate (50 mL×3) and the combined organic phases were washed with brine, dried over anhydrous Na2SO4 and concentrated to give a residue, which was purified by flash silica gel chromatography (ISCO®; 20 g SepaFlash® Silica Flash Column, Eluent of 0-77% Ethyl acetate/Petroleum ether gradient @80 mL/min) to afford Intermediate Compound 22 (295 mg, 33% yield) as a white solid.

LCMS: Rt=0.434 min; (ESI positive ion) m/z: 461.9 (M+H)+(calculated: 462.02).

### Intermediate Compound 23:

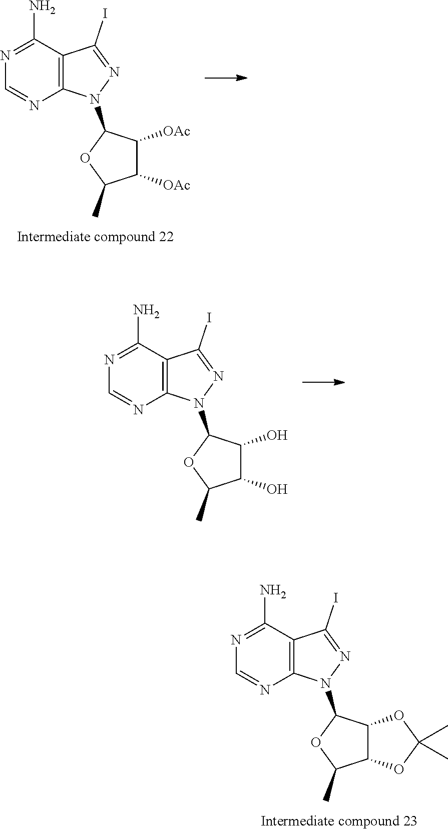

To a solution of Intermediate Compound 22 (295 mg, 639.62 μmol, 1 eq) in MeOH (3 mL) was added NH3·H2O (2.73 g, 21.81 mmol, 3 mL, 28% purity, 34.10 eq). The mixture was stirred at 20° C. for 0.5 hr. The reaction mixture was filtered and filtrate was concentrated under reduced pressure to give a residue of the corresponding diol as a white solid. The residue was used into the next step without further purification (260 mg, crude).

LCMS: Rt=0.220 min; (ESI positive ion) m/z: 377.9 (M+H)+(calculated: 378.00).

To a solution of the above diol (60 mg, 689.40 μmol, 1 eq) in acetone (3 mL) was added TsOH·H2O (1.31 g, 6.89 mmol, 10 eq) at 0° C. The mixture was stirred at 20° C. for 3 hr. The reaction mixture was quenched with saturated NaHCO3 (20 mL) under ice-water bath cooling. The aqueous phase was extracted with ethyl acetate (20 mL×3) and the combined organic phases were washed with brine, dried over anhydrous Na2SO4 and concentrated to give a residue. The residue was purified by Prep-TLC (SiO2, Petroleum/Ethyl acetate=1/1) to afford Intermediate Compound 23 (215 mg, 75% yield) as a colorless oil.

LCMS: Rt=0.443 min; (ESI positive ion) m/z: 417.9 (M+H)+(calculated: 418.03).

### Intermediate Compound 24:

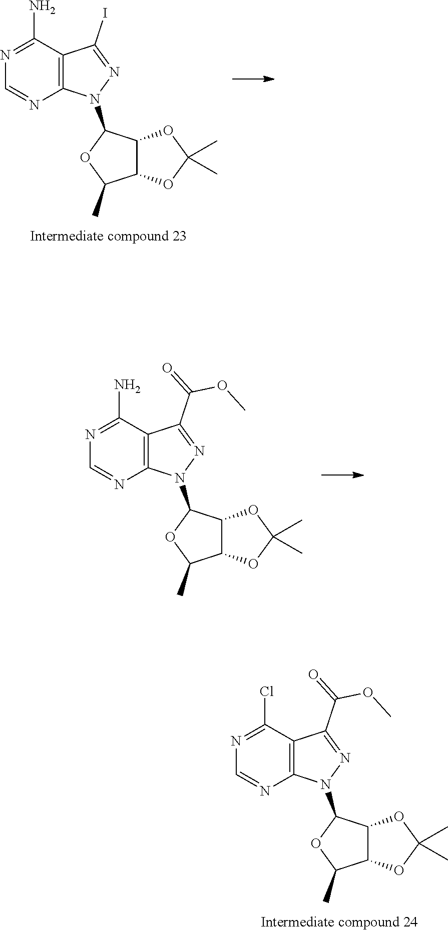 [SMILES: C[C@H]1O[C@@H](n2nc(I)c3c(N)ncnc32)[C@@H]2OC(C)(C)O[C@@H]21 ; COC(=O)c1nn([C@@H]2O[C@H](C)[C@H]3OC(C)(C)O[C@H]32)c2ncnc(N)c12 ; COC(=O)c1nn([C@@H]2O[C@H](C)[C@H]3OC(C)(C)O[C@H]32)c2ncnc(Cl)c12]

To a solution of Intermediate Compound 23 (210 mg, 503.35 μmol, 1 eq) in MeOH (10 mL) were added Pd(dppf)Cl2·CH2Cl2 (41.11 mg, 50.34 μmol, 0.1 eq) and TEA (152.80 mg, 1.51 mmol, 210.18 μL, 3 eq) under N2 atmosphere. The mixture was stirred at 80° C. for 16 hr under CO (50 PSI) atmosphere. The mixture was filtered and concentrated under vacuum to give a residue. The residue was purified by prep-TLC (SiO2, Petroleum/Ethyl acetate=1/1) to afford the corresponding ester (174 mg, 99% yield) as a yellow oil.

LCMS: Rt=0.358 min; (ESI positive ion) m/z: 350.2 (M+H)+(calculated: 350.14).

To a solution of the above ester (100 mg, 286.25 μmol, 1 eq) in THE (1 mL) was added CuCl2 (57.73 mg, 429.38 μmol, 1.5 eq) and amyl nitrite (167.67 mg, 1.43 mmol, 5 eq). The mixture was stirred at 50° C. for 12 hr. The reaction mixture was filtered and filtrate was concentrated under reduced pressure to give a residue. The residue was purified by Prep-TLC (SiO2, Petroleum/Ethyl acetate=1/1) to afford Intermediate Compound 24 (7 mg, 7% yield) as a colorless oil.

LCMS: Rt=0.518 min; (ESI positive ion) m/z: 369.0 (M+H)+(calculated: 369.09).

### Intermediate Compound 25:

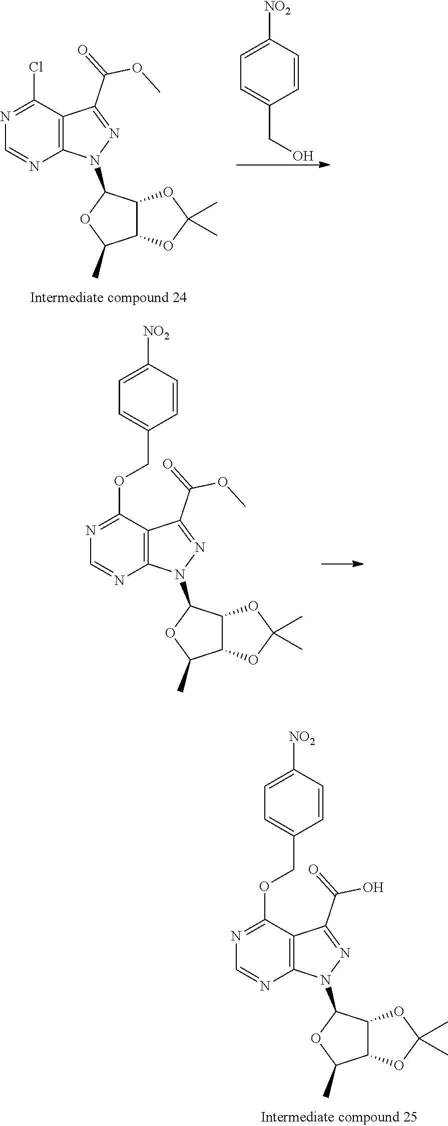 [SMILES: COC(=O)c1nn([C@@H]2O[C@H](C)[C@H]3OC(C)(C)O[C@H]32)c2ncnc(Cl)c12 ; O=[N+]([O-])c1ccc(CO)cc1 ; COC(=O)c1nn([C@@H]2O[C@H](C)[C@H]3OC(C)(C)O[C@H]32)c2ncnc(OCc3ccc([N+](=O)[O-])cc3)c12 ; C[C@H]1O[C@@H](n2nc(C(=O)O)c3c(OCc4ccc([N+](=O)[O-])cc4)ncnc32)[C@@H]2OC(C)(C)O[C@@H]21]

To a solution of Intermediate Compound 24 (15 mg, 40.68 μmol, 1 eq) in toluene (1 mL) was added (4-nitrophenyl)methanol (7.47 mg, 48.81 μmol, 1.2 eq), Xantphos (4.71 mg, 8.14 μmol, 0.2 eq), Cs2CO3 (33.13 mg, 101.69 μmol, 2.5 eq) and Pd2(dba)3 (3.72 mg, 4.07 μmol, 0.1 eq) under N2 atmosphere. The mixture was stirred at 80° C. for 0.5 hr. The reaction mixture was filtered and the filtrate was concentrated under reduced pressure to give a residue. The residue was purified by Prep-TLC (SiO2, Petroleum/Ethyl acetate=2/1) to afford the corresponding ether (13 mg, 66% yield) as a yellow solid.

LCMS: Rt=0.548 min; (ESI positive ion) m/z: 486.2 (M+H)+(calculated: 486.15).

To a solution of the above ether (13 mg, 26.78 μmol, 1 eq) in MeOH (0.2 mL), THE (0.2 mL) and H2O (0.2 mL) was added NaOH (2 M, 26.78 μL, 2 eq) at 0° C. The mixture was stirred at 0° C. for 10 min. An aqueous solution of 2N HCl was then added to the reaction mixture at 0° C. until pH=5, and then the mixture was diluted with water (10 mL) and extracted with ethyl acetate (10 mL×3). The combined organic layers were washed with brine (20 mL), dried over Na2SO4, filtered and concentrated under reduced pressure to give crude Intermediate Compound 25 (12 mg, crude) as a yellow solid, which was not further purified.

LCMS: Rt=0.556 min; (ESI positive ion) m/z: 472.1 (M+H)+(calculated: 472.14).

### Intermediate Compound 26:

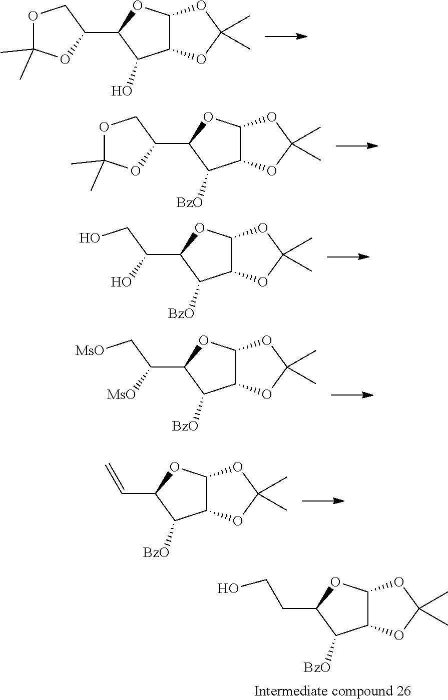

To a mixture of (3aR,5S,6R,6aR)-5-((R)-2,2-dimethyl-1,3-dioxolan-4-yl)-2,2-dimethyltetrahydrofuro[2,3-d][1,3]dioxol-6-ol (9.5 g, 36.50 mmol, 1 eq) and pyridine (8.66 g, 109.50 mmol, 8.84 mL, 3 eq) in 1,2-dichloroethane (100 mL) was added benzoyl chloride (7.70 g, 54.75 mmol, 6.36 mL, 1.5 eq) at 0° C. The mixture was stirred at 25° C. for 16 hr. The mixture was then poured into ice-water (500 mL) and stirred for 1 min. The aqueous phase was extracted with DCM (200 mL×2). The combined organic phase was dried with anhydrous Na2SO4, filtered and concentrated under vacuum to give a residue. The residue was purified by column chromatography (SiO2, Petroleum ether/Ethyl acetate=100/1 to 10/1) to afford the corresponding ester (11.8 g, 81% yield) as a colorless oil.

LCMS: Rt=0.432 min; (ESI positive ion) m/z: 387.0 (M+Na)+(calculated: 387.15).

A mixture of the above ester compound (11.8 g, 32.38 mmol, 1 eq) in acetic acid (90 mL) and water (10 mL) was stirred at 60° C. for 45 min. The mixture was concentrated under vacuum to give a residue. The residue was purified by column chromatography (SiO2, Petroleum ether/Ethyl acetate=100/1 to 1/1) to afford the corresponding diol (5.1 g, 48% yield) as a white solid.

To a solution of the above diol (5.1 g, 15.72 mmol, 1 eq) and TEA (4.77 g, 47.17 mmol, 6.57 mL, 3 eq) in DCM (60 mL) was added MsCl (4.50 g, 39.31 mmol, 3.04 mL, 2.5 eq) at 0° C. The mixture was stirred at 0° C. for 0.5 hr. The mixture was poured into ice-water (500 mL) and extracted with DCM (200 mL×2). The combined organic phase was dried with anhydrous Na2SO4, filtered and concentrated under vacuum to afford the corresponding mesylate (7.5 g, crude) as a yellow oil, which was used directly in the next step.

A mixture of the above mesylate compound (7.5 g, 15.61 mmol, 1 eq) and NaI (15.21 g, 101.46 mmol, 6.5 eq) in 3-pentanone (70 mL) was stirred at 100° C. for 2.5 hr. The mixture was poured into an ice-cold saturated aqueous solution of Na2SO3 (500 mL) and stirred for 1 min. The aqueous phase was extracted with DCM (200 mL×2). The combined organic phase was dried with anhydrous Na2SO4, filtered and concentrated under vacuum. The residue was purified by column chromatography (SiO2, Petroleum ether/Ethyl acetate=100/1 to 5/1) to afford the corresponding vinyl compound (3.9 g, 83% yield) as a colorless oil.

LCMS: Rt=0.438 min; (ESI positive ion) m/z: 313.0 (M+Na)+(calculated: 313.12).

To a solution of the above vinyl compound (3.9 g, 13.43 mmol, 1 eq) in THE (50 mL) was added BH3-Me2S (10 M, 4.03 mL, 3 eq) at 0° C. The mixture was stirred at 0° C. for 16 hr. Then a solution of NaHCO3 (6.85 g, 81.58 mmol, 3.17 mL, 6.07 eq) in H2O (25 mL) was added to the mixture at −10° C., and then H2O2 (13.81 g, 121.77 mmol, 11.70 mL, 30% purity, 9.06 eq) was added to the mixture at 0° C. The resulting mixture was stirred at 25° C. for 2 hr. The mixture was poured into a saturated solution of Na2SO3 (500 mL). The aqueous phase was extracted with ethyl acetate (200 mL×2). The combined organic phase was washed with brine (500 mL), dried with anhydrous Na2SO4, filtered and concentrated under vacuum to give a residue. The residue was purified by column chromatography (SiO2, Petroleum ether/Ethyl acetate=100/1 to 1/1) to afford Intermediate Compound 26 (1.7 g, 41% yield) as a colorless oil.

1H NMR (400 MHz, CDCl₃) δ=8.08 (d, J=7.2 Hz, 2H), 7.64-7.58 (m, 1H), 7.50-7.44 (m, 2H), 5.90 (d, J=3.8 Hz, 1H), 4.95 (t, J=4.4 Hz, 1H), 4.75 (dd, J=4.8, 9.2 Hz, 1H), 4.45 (dt, J=3.6, 8.8 Hz, 1H), 3.86 (t, J=5.8 Hz, 2H), 2.09-2.03 (m, 1H), 1.93-1.85 (m, 1H), 1.57 (s, 3H), 1.34 (s, 3H).

### Intermediate Compound 27:

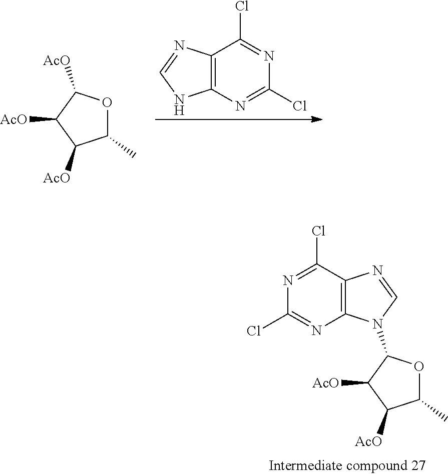 [SMILES: CC(=O)O[C@@H]1O[C@H](C)[C@@H](OC(C)=O)[C@H]1OC(C)=O ; Clc1nc(Cl)c2nc[nH]c2n1 ; CC(=O)O[C@@H]1[C@H](OC(C)=O)[C@@H](C)O[C@H]1n1cnc2c(Cl)nc(Cl)nc21]

To a mixture of 2,6-dichloro-9H-purine (1 g, 3.84 mmol, 1 eq) and (2S,3R,4R,5R)-5-methyltetrahydrofuran-2,3,4-triyl triacetate (871.51 mg, 4.61 mmol, 1.2 eq) in 1,2-dichloroethane (15 mL) was added bis(trimethylsilyl)acetamide (1.56 g, 7.69 mmol, 1.90 mL, 2 eq). The mixture was stirred at 80° C. for 0.5 hr until the solution became clear. After 0.5 hr, the mixture was cooled to room temperature, and then TMSOTf (1.11 g, 5.00 mmol, 902.65 μL, 1.3 eq) was added to the mixture dropwise. The mixture was stirred at 80° C. for 12 hr. The reaction mixture was then poured into a saturated aqueous solution of NaHCO3 (30 mL) and extracted with DCM (20 mL×3). The organic layer was washed with brine, dried with Na2SO4, filtered and concentrated to give a residue. The residue was purified by flash silica gel chromatography (ISCO®; 12 g SepaFlash® Silica Flash Column, Eluent of 35-45% Ethyl acetate/Petroleum ether gradient @40 mL/min). The purified solution was concentrated to afford Intermediate Compound 27 (1.2 g, 80% yield) as a colorless gum.

LCMS: Rt=0.874 min; (ESI positive ion) m/z: 389.0 (M+Na)+(calculated: 389.03).

1H NMR (400 MHz, CDCl₃) δ=8.22 (s, 1H), 6.12 (d, J=5.3 Hz, 1H), 5.82 (t, J=5.5 Hz, 1H), 5.37-5.32 (m, 1H), 4.43-4.34 (m, 1H), 2.17 (s, 3H), 2.10 (s, 3H), 1.55 (d, J=6.6 Hz, 3H).

### Intermediate Compound 28:

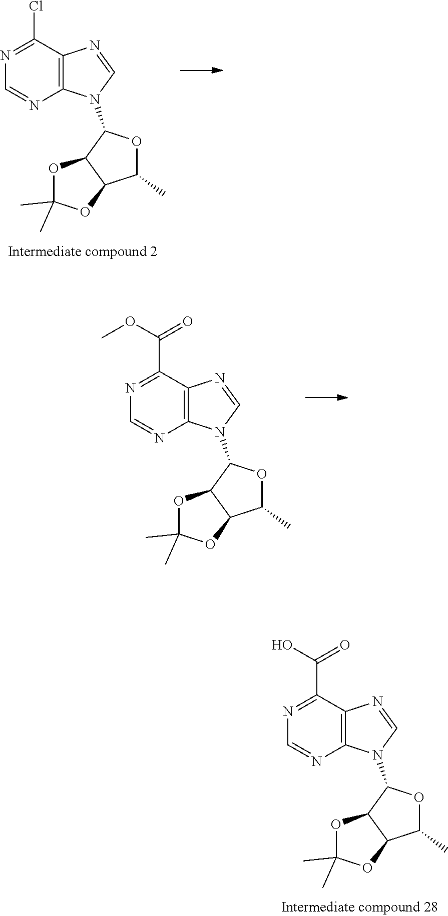

To a solution of Intermediate Compound 2 (500 mg, 1.61 mmol, 1 eq) in MeOH (10 mL) was added Pd(dppf)Cl2·CH2Cl2 (131.40 mg, 160.91 μmol, 0.1 eq) and TEA (488.47 mg, 4.83 mmol, 671.90 μL, 3 eq). Then CO (50 Psi) was introduced. The mixture was stirred at 80° C. for 16 hours under CO. The reaction mixture was filtered and concentrated under reduced pressure to give a residue. The residue was purified by Prep-TLC (SiO2, Petroleum ether:Ethyl acetate=1:1) to afford the corresponding ester compound (300 mg, 56% yield) as a brown solid.

LCMS: Rt=0.879 min; (ESI positive ion) m/z: 335.1 (M+Na)+(calculated: 335.13).

To a solution of the above ester compound (300 mg, 897.33 μmol, 1 eq) in MeOH (4 mL), THE (4 mL), and H2O (4 mL) was added NaOH (2 M, 897.33 μL, 2 eq). The mixture was stirred at 0° C. for 10 min. To the reaction mixture was added a solution of 2N HCl (4 mL) at 0° C. until the pH measured 6, and then the mixture was diluted with water (50 mL) and extracted with ethyl acetate (50 mL×3). The combined organic layers were washed with brine (40 mL), dried over Na2SO4, filtered and concentrated under reduced pressure to afford Intermediate Compound 28 (200 mg, crude) as a yellow solid.

LCMS: Rt=0.684 min; (ESI positive ion) m/z: 321.0 (M+H)+(calculated: 321.11).

### Intermediate Compound 29:

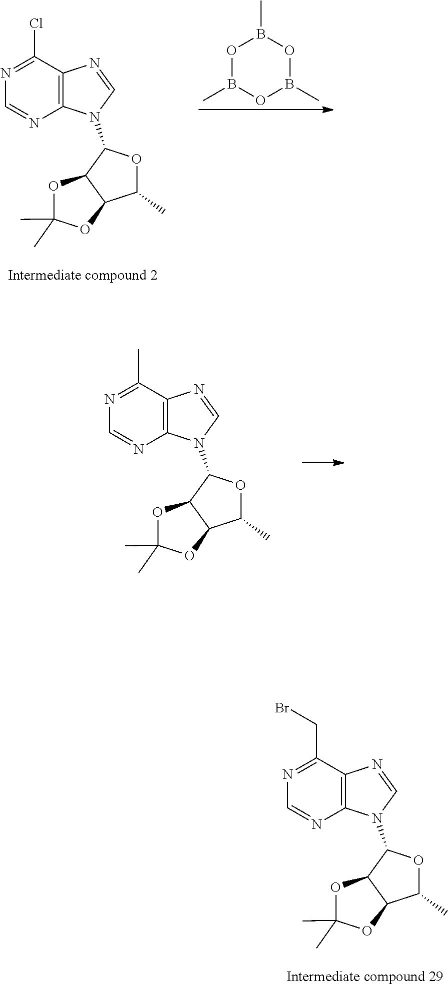

To a solution of Intermediate Compound 2 (1 g, 3.22 mmol, 1 eq in dioxane (20 mL) and water (2 mL) were added K2CO3 (1.33 g, 9.65 mmol, 3 eq, 2,4,6-trimethyl-1,3,5,2,4,6-trioxatriborinane (2.02 g, 8.05 mmol, 2.25 mL, 50% purity, 2.5 eq and Pd(dppf)Cl2·CH2Cl2 (262.81 mg, 321.82 μmol, 0.1 eq. The mixture was stirred at 100° C. for 12 hr under N2 atmosphere. The reaction mixture was diluted with EtOAc (60 mL) and filtered. The filtrate was concentrated to give a residue. The residue was purified by flash silica gel chromatography (ISCO®; 20 g SepaFlash® Silica Flash Column, Eluent of 0-60% Ethyl acetate/Petroleum ether gradient @80 mL/min) to give the corresponding methylated compound (770 mg, 82% yield) as yellow oil.

LCMS: Rt=0.285 min; (ESI positive ion) m/z: 291.0 (M+Na)+(calculated: 291.14).

To a solution of the above methylated compound (770 mg, 2.65 mmol, 1 eq in THF (20 mL) was added AIBN (43.55 mg, 265.23 μmol, 0.1 eq and NBS (566.46 mg, 3.18 mmol, 1.2 eq at 0° C. The reaction mixture was stirred at 70° C. for 36 hr under N2 atmosphere. The solvent was removed under reduced pressure to give a residue. The residue was purified by flash silica gel chromatography (ISCO®; 20 g SepaFlash® Silica Flash Column, Eluent of 0-60% Ethyl acetate/Petroleum ether gradient @40 mL/min) to afford Intermediate Compound 29 (107 mg, 11% yield) as a yellow gum.

LCMS: Rt=0.354 min; (ESI positive ion) m/z: 370.9 (M+Na)+(calculated: 371.05).

### Intermediate Compound 30:

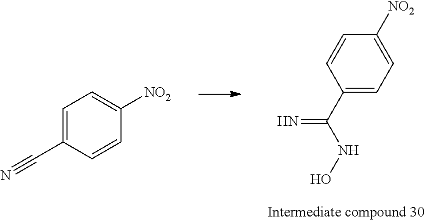 [SMILES: N#Cc1ccc([N+](=O)[O-])cc1 ; N=C(NO)c1ccc([N+](=O)[O-])cc1]

To a solution of 4-nitrobenzonitrile (2.00 g, 13.50 mmol) in ethanol (40 mL) was added NH2OH·HCl (3.8 g, 54.68 mmol) and Na2CO3 (4.4 g, 41.51 mmol). The mixture was degassed and purged with N2 for 3 times and then stirred at 85° C. for 2 hr under N2 atmosphere. The reaction mixture was concentrated under vacuum to afford Intermediate Compound 30 (2.50 g, crude) as a yellow solid.

1H NMR (400 MHz, DMSO-d6) δ=10.12 (s, 1H), 8.25-8.20 (m, 2H), 7.94 (d, J=9.0 Hz, 2H), 6.05 (s, 2H).

### Intermediate Compound 31:

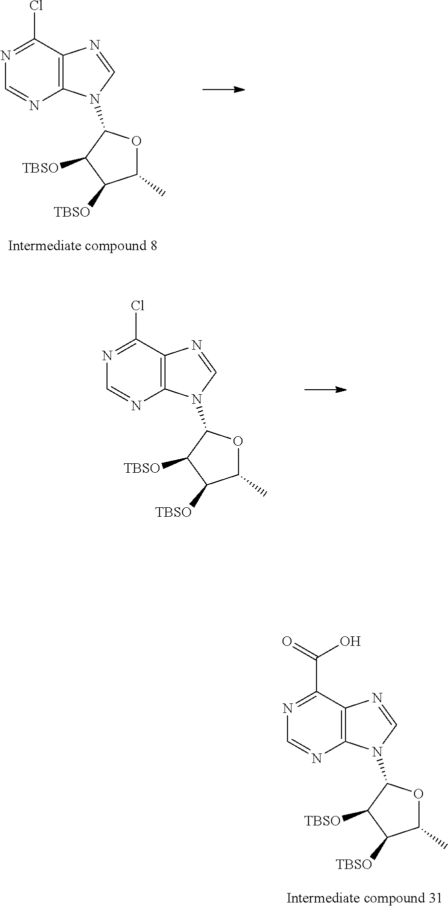 [SMILES: C[C@H]1O[C@@H](n2cnc3c(Cl)ncnc32)[C@H](O[Si](C)(C)C(C)(C)C)[C@@H]1O[Si](C)(C)C(C)(C)C ; C[C@H]1O[C@@H](n2cnc3c(Cl)ncnc32)[C@H](O[Si](C)(C)C(C)(C)C)[C@@H]1O[Si](C)(C)C(C)(C)C ; C[C@H]1O[C@@H](n2cnc3c(C(=O)O)ncnc32)[C@H](O[Si](C)(C)C(C)(C)C)[C@@H]1O[Si](C)(C)C(C)(C)C]

Intermediate Compound 31 was synthesized from Intermediate Compound 8 following the protocols described for Intermediate Compound 28.

LCMS: Rt=0.712 min; (ESI positive ion) m/z: 509.4 (M+H)+(calculated: 509.25).

### Intermediate Compound 32:

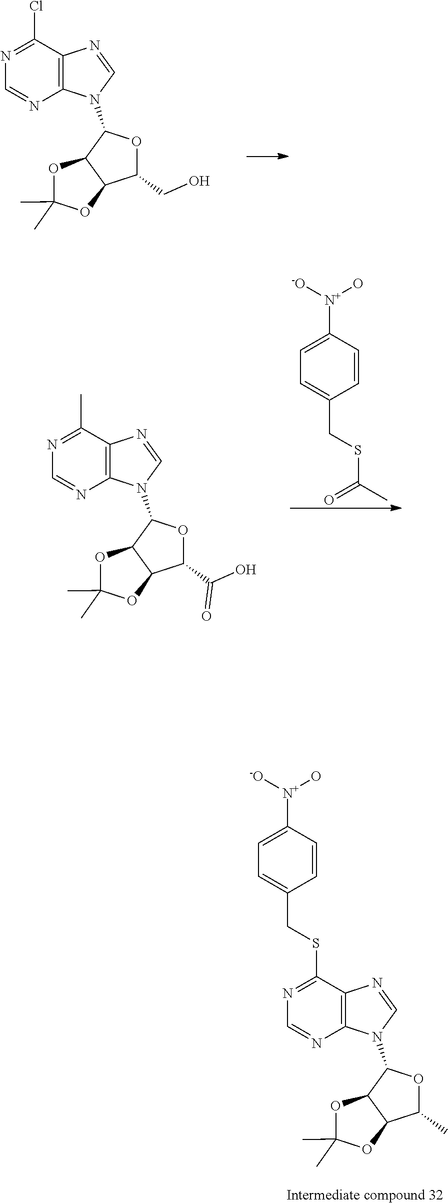

To a solution of ((3aR,4R,6R,6aR)-6-(6-chloro-9H-purin-9-yl)-2,2-dimethyltetrahydrofuro[3,4-d][1,3]dioxol-4-yl)methanol (500 mg, 1.53 mmol, 1 eq) and sodium periodate (1.34 g, 6.27 mmol, 347.67 μL, 4.1 eq) in water (7.5 mL), MeCN (5 mL) and carbon tetrachloride (5 mL) was added ruthenium(III) chloride trihydrate (88.03 mg, 336.66 μmol, 0.22 eq). The mixture was stirred at 20° C. for 12 hr. The solvent was removed under reduced pressure to give a residue. The residue was purified by reversed-phase HPLC (0.1% NH3·H2O condition). The purified solution was lyophilized to afford the corresponding acid compound (453 mg, 87% yield) as black-brown solid.

1H NMR (400 MHz, DMSO-d6) δ=9.39 (s, 1H), 8.76 (s, 1H), 7.30 (br s, 1H), 6.31 (d, J=1.7 Hz, 1H), 5.23 (dd, J=1.5, 5.8 Hz, 1H), 5.11 (dd, J=1.2, 5.9 Hz, 1H), 4.44 (s, 1H), 1.53 (s, 3H), 1.32 (s, 3H).

A solution of the above acid compound (240 mg, 704.39 μmol, 1 eq) and S-(4-nitrobenzyl) ethanethioate (163.67 mg, 774.83 μmol, 1.1 eq) in MOH (2.4 mL) and THF (0.6 mL) was stirred at 0° C. while K2CO3 (204.44 mg, 1.48 mmol, 2.1 eq) was added in one portion. The mixture was stirred at 0° C. for 2 hr. To the reaction mixture was added MeOH (20 mL) and the mixture was filtered. The filtrate was concentrated to give a residue. The residue was purified by Prep-HPLC (column: Waters Xbridge 150×25 mm×5 μm; mobile phase: [water (0.05% ammonia hydroxide v/v)-ACN]; B %: 10%-40%, 9 min). The purified solution was lyophilized to afford Intermediate Compound 32 (124 mg, 37% yield) as a red solid.

LCMS: Rt=0.717 min; (ESI positive ion) m/z: 474.1 (M+H)+(calculated: 474.10).

1H NMR (400 MHz, DMSO-d6) δ=9.39 (s, m, 8.74 (s, 1H), 8.16 (d, J=8.8 Hz, 2H), 7.75 (d, J=8.6 Hz, 2H), 6.25 (d, J=1.8 Hz, 1H), 5.13-4.97 (m, 2H), 4.80 (s, 2H), 4.38 (s, 1H), 1.52 (s, 3H), 1.30 (s, 3H).

### Intermediate Compound 33:

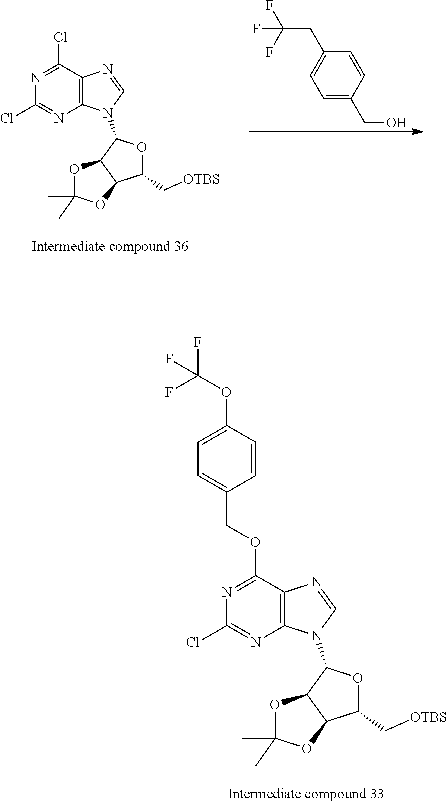 [SMILES: CC1(C)O[C@@H]2[C@H](O1)[C@@H](CO[Si](C)(C)C(C)(C)C)O[C@H]2n1cnc2c(Cl)nc(Cl)nc21 ; OCc1ccc(CC(F)(F)F)cc1 ; CC1(C)O[C@@H]2[C@H](O1)[C@@H](CO[Si](C)(C)C(C)(C)C)O[C@H]2n1cnc2c(OCc3ccc(OC(F)(F)F)cc3)nc(Cl)nc21]

To a mixture of (4-(trifluoromethoxy)phenyl)methanol (323.30 mg, 1.68 mmol, 243.08 μL, 0.8 eq) and Intermediate Compound 36 (1 g, 2.10 mmol, 1 eq) in dioxane (15 mL) was added Cs2CO3 (1.71 g, 5.26 mmol, 2.5 eq), Xantphos (243.40 mg, 420.66 μmol, 0.2 eq) and Pd2(dba)3 (192.60 mg, 210.33 μmol, 0.1 eq). The mixture was degassed and purged with N2 3 times, and then the mixture was stirred at 80° C. for 1.5 hr under N2 atmosphere. The reaction mixture was filtrated, evaporated and the residue purified by column chromatography (SiO2, Petroleum ether/Ethyl acetate=100/0 to 2/1) to afford Intermediate Compound 33 (1 g, 64% yield) as a yellow oil.

LCMS: Rt=1.201 min; (ESI positive ion) m/z: 630.9 (M+H)+(calculated: 631.19).

Intermediate compound 34:

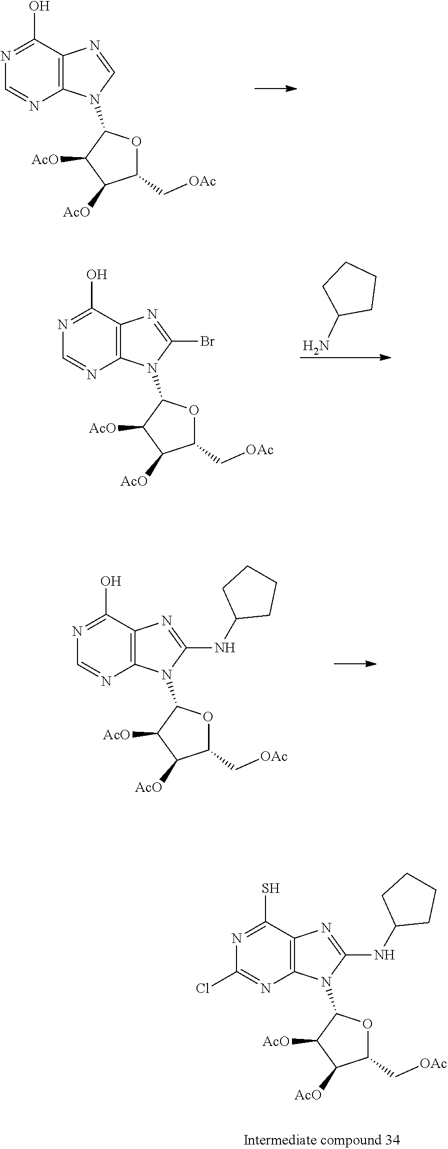 [SMILES: CC(=O)OC[C@H]1O[C@@H](n2cnc3c(O)ncnc32)[C@H](OC(C)=O)[C@@H]1OC(C)=O ; CC(=O)OC[C@H]1O[C@@H](n2c(Br)nc3c(O)ncnc32)[C@H](OC(C)=O)[C@@H]1OC(C)=O ; NC1CCCC1 ; CC(=O)OC[C@H]1O[C@@H](n2c(NC3CCCC3)nc3c(O)ncnc32)[C@H](OC(C)=O)[C@@H]1OC(C)=O ; CC(=O)OC[C@H]1O[C@@H](n2c(NC3CCCC3)nc3c(S)nc(Cl)nc32)[C@H](OC(C)=O)[C@@H]1OC(C)=O]

To a 10% aqueous Na2HPO4 solution (55 mL) at 20° C. was added Br2 (9.30 g, 58.19 mmol, 3 mL, 22.95 eq) and the mixture was stirred vigorously for 15 min until most of the bromine had dissolved. The decanted bromine solution was added to a solution of (2R,3R,4R,5R)-2-(acetoxymethyl)-5-(6-hydroxy-9H-purin-9-yl)tetrahydrofuran-3,4-diyl diacetate (1 g, 2.54 mmol, 1 eq) in dioxane (38 mL). The reaction mixture was stirred at 20° C. for 36 hr. After cooling in an ice/water bath, an aqueous NaHSO3 solution (2 N) was added dropwise until the solution became colorless. The water layer was extracted with DCM (3×30 mL). The organic layer was washed with an aqueous NaHSO3 solution (0.2 N, 50 mL) and water (50 mL), dried over Na2SO4, filtered, and concentrated under vacuum to give a residue. The residue was purified by column chromatography (SiO2, Petroleum ether/Ethyl acetate=1/0 to 0/1) to afford the corresponding brominated compound (500 mg, 1.06 mmol, 42% yield) as a white solid.

LCMS: Rt=0.370 min; (ESI positive ion) m/z: 474.90 (M+H)+(calculated: 475.02).

1H NMR (400 MHz, CDCl₃) δ=12.80 (br s, 1H), 8.18 (s, 1H), 6.24 (dd, J=4.8, 5.8 Hz, 1H), 6.12 (d, J=4.8 Hz, 1H), 5.79 (t, J=5.8 Hz, 1H), 4.50 (dd, J=3.6, 11.8 Hz, 1H), 4.43-4.37 (m, 1H), 4.36-4.30 (m, 1H), 2.17 (s, 3H), 2.12 (s, 3H), 2.08 (s, 3H).

To a solution of the above brominated compound (500 mg, 1.06 mmol, 1 eq) in dioxane (10 mL) was added cyclopentanamine (1.21 g, 14.26 mmol, 1.41 mL, 13.5 eq). The mixture was stirred at 80° C. for 36 hr. The solvent was removed under reduced pressure to give a residue. The residue was dissolved in pyridine (4 mL). To this solution was added acetic anhydride (1.08 g, 10.57 mmol, 989.58 μL, 10 eq) and DMAP (12.91 mg, 105.66 μmol, 0.1 eq). The mixture was stirred at 20° C. for 12 hr. The reaction was quenched by addition of MeOH (2 mL), and the mixture was concentrated under vacuum. The resultant residue was dissolved in DCM (30 mL) and washed with an aqueous NaHCO3 solution (20 mL) and H2O (20 mL). The organic layer was dried over Na2SO4 and concentrated to give a residue. The residue was purified by flash silica gel chromatography (ISCO®; 20 g SepaFlash® Silica Flash Column, Eluent of 0-12% MeOH/DCM gradient @80 mL/min) to afford the corresponding amine compound (770 mg, crude) as a red oil.

LCMS: Rt=0.309 min; (ESI positive ion) m/z: 478.20 (M+H)+(calculated: 478.19).

To a solution of the above amine compound (570 mg, 1.19 mmol, 1 eq) in pyridine (15 mL) was added phosphorus pentasulfide (2.09 g, 9.40 mmol, 999.59 μL, 7.87 eq). The mixture was stirred at 120° C. for 12 hr. After evaporation of the solvent under vacuum, the residual solvent was removed by co-evaporation with MeOH (30 mL). Water was added and the resulting mixture was stirred for 1 hr at 50° C. After extraction with EtOAc (30 mL×3), the combined organic layers were dried with Na2SO4 and filtered and the solvent was evaporated to give a residue. The residue was purified by Prep-TLC (SiO2, DCM:MeOH=10:1) to afford Intermediate Compound 34 (148 mg, crude) as a red solid.

LCMS: Rt=0.430 min; (ESI positive ion) m/z: 494.10 (M+H)+(calculated: 494.16).

### Intermediate Compound 35:

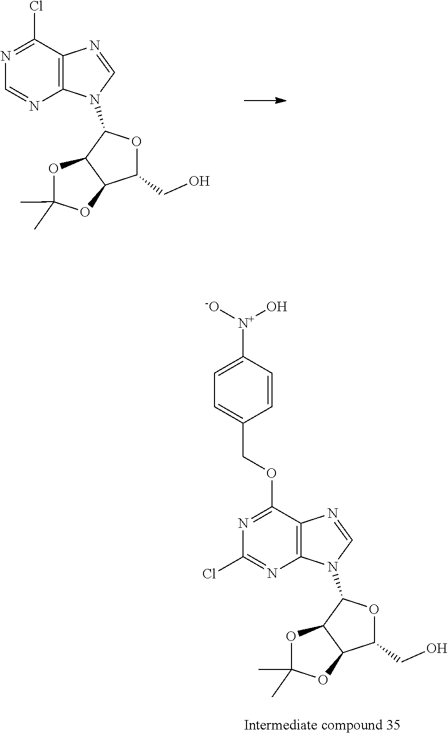

A solution of ((3aR,4R,6R,6aR)-6-(6-chloro-9H-purin-9-yl)-2,2-dimethyltetrahydrofuro[3,4-d][1,3]dioxol-4-yl)methanol (1 g, 3.06 mmol, 1 eq) and S-(4-nitrobenzyl) ethanethioate (711.16 mg, 3.37 mmol, 1.1 eq) in MeOH (4 mL) and THE (1 mL) was stirred at 0° C., while K2CO3 (888.28 mg, 6.43 mmol, 2.1 eq) was added in one portion. The mixture was stirred at 0° C. for 2 hr. The reaction mixture was filtered. The filtrate was concentrated to give a residue. The residue was purified by Prep-HPLC (column: Phenomenex luna C18 150×40 mm×15 μm; mobile phase:[water(0.225% FA)-ACN]; B %: 40%-70%, 13 min). The purified solution was lyophilized to afford Intermediate Compound 35 (450 mg, 32% yield) as a red solid.

LCMS: Rt=0.902 min; (ESI positive ion) m/z: 460.20 (M+H)+(calculated: 460.12).

### Intermediate Compound 36:

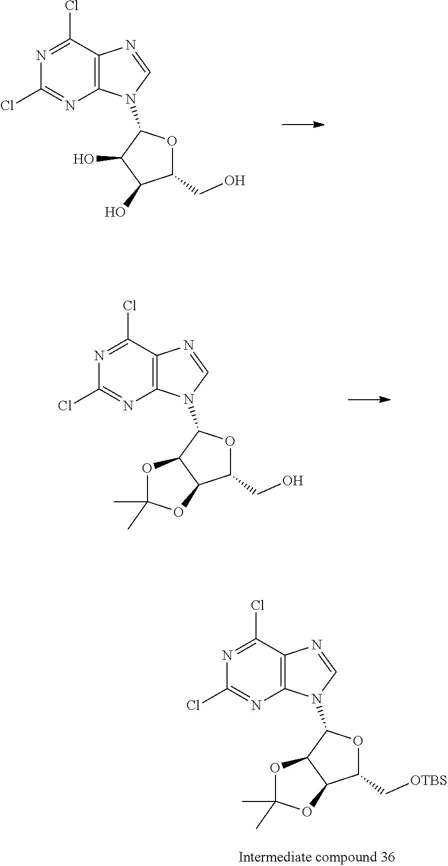 [SMILES: OC[C@H]1O[C@@H](n2cnc3c(Cl)nc(Cl)nc32)[C@H](O)[C@@H]1O ; CC1(C)O[C@@H]2[C@H](O1)[C@@H](CO)O[C@H]2n1cnc2c(Cl)nc(Cl)nc21 ; CC1(C)O[C@@H]2[C@H](O1)[C@@H](CO[Si](C)(C)C(C)(C)C)O[C@H]2n1cnc2c(Cl)nc(Cl)nc21]

To a solution of 2,6-Dichloro-9-β-D-ribofuranosyl-9H-purine (4 g, 12.46 mmol, 1 eq) in acetone (100 mL) was added 4-methylbenzenesulfonic acid hydrate (23.69 g, 124.57 mmol, 10 eq). The mixture was stirred at 20° C. for 3 hr. The reaction mixture was diluted with water (100 mL) and extracted with ethyl acetate (100 mL×2). The combined organic layers were washed with brine (200 mL), dried over Na2SO4, filtered and concentrated under reduced pressure to give a residue. The residue was purified by column chromatography (SiO2, Petroleum ether/Ethyl acetate=100/0 to 0/1), TLC (SiO2, Petroleum ether/Ethyl acetate=0:1, Rf=0.6) to afford the corresponding acetonide compound (2.8 g, 62% yield) as a white solid.

1H NMR (400 MHz, CDCl₃) δ=8.31 (s, 1H), 5.99 (d, J=4.4 Hz, 1H), 5.20-5.15 (m, 1H), 5.13-5.09 (m, 1H), 4.53 (d, J=1.6 Hz, 1H), 4.00 (dd, J=2.0, 12.4 Hz, 1H), 3.85 (dd, J=2.4, 12.8 Hz, 1H), 1.65 (s, 3H), 1.39 (s, 3H).

To a mixture of the above acetonide compound (2.3 g, 6.37 mmol, 1 eq) in DMF (40 mL) was added TEA (1.93 g, 19.10 mmol, 2.66 mL, 3 eq) and tert-butyldimethylsilyl chloride (1.92 g, 12.74 mmol, 1.56 mL, 2 eq). Then the mixture was stirred maintaining a temperature from about 0° C. to about 20° C. for 2 hr under N2 atmosphere. The reaction mixture was diluted with water (60 mL) and extracted with ethyl acetate (60 mL×2). The combined organic layers were washed with brine (80 mL×2), dried over Na2SO4, filtered and concentrated under reduced pressure to give a residue. The residue was purified by column chromatography (SiO2, Petroleum ether/Ethyl acetate=100/0 to 1/1) to give Intermediate Compound 36 (3 g, 94% yield) as a yellow oil.

LCMS: Rt=1.095 min; (ESI positive ion) m/z: 475.1 (M+H)+(calculated: 475.13).

### Intermediate Compound 37:

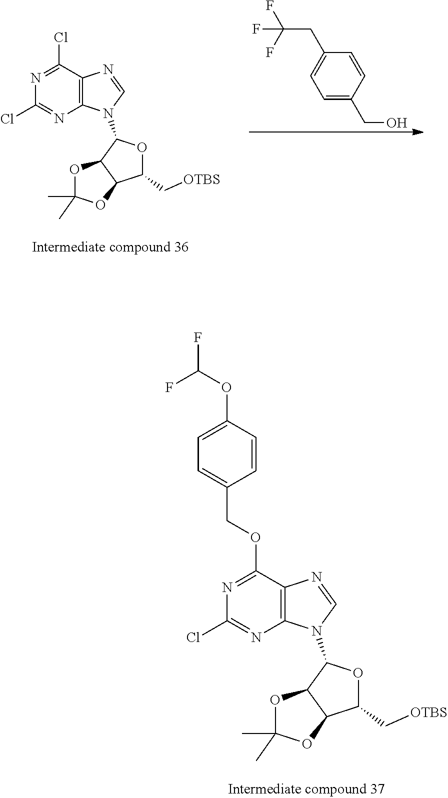 [SMILES: CC1(C)O[C@@H]2[C@H](O1)[C@@H](CO[Si](C)(C)C(C)(C)C)O[C@H]2n1cnc2c(Cl)nc(Cl)nc21 ; OCc1ccc(CC(F)(F)F)cc1 ; CC1(C)O[C@@H]2[C@H](O1)[C@@H](CO[Si](C)(C)C(C)(C)C)O[C@H]2n1cnc2c(OCc3ccc(OC(F)F)cc3)nc(Cl)nc21]

A mixture of Intermediate Compound 36 (300 mg, 630.99 μmol, 1 eq), (4-(difluoromethoxy)phenyl)methanol (87.91 mg, 504.80 μmol, 0.8 eq), Xantphos (73.02 mg, 126.20 mol, 0.2 eq), Pd2(dba)3 (57.78 mg, 63.10 μmol, 0.1 eq) and Cs2CO3 (513.98 mg, 1.58 mmol, 2.5 eq) in dioxane (1 mL) was degassed and purged with N2 3 times. The mixture was stirred at 80° C. for 1.5 hours under N2 atmosphere. The reaction mixture was filtered and concentrated under reduced pressure to give a residue. The residue was purified by column chromatography (SiO2, Petroleum ether:Ethyl acetate=1:0 to 2:1) to afford Intermediate Compound 37 (240 mg) as a white solid.

LCMS: Rt=1.199 min; (ESI positive ion) m/z: 613.3 (M+H)+(calculated: 613.20).

### Intermediate Compound 38:

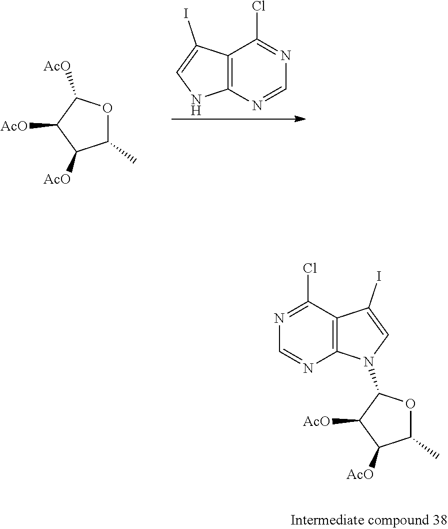 [SMILES: CC(=O)O[C@@H]1O[C@H](C)[C@@H](OC(C)=O)[C@H]1OC(C)=O ; Clc1ncnc2[nH]cc(I)c12 ; CC(=O)O[C@@H]1[C@H](OC(C)=O)[C@@H](C)O[C@H]1n1cc(I)c2c(Cl)ncnc21]

To a solution of 4-chloro-5-iodo-7H-pyrrolo[2,3-d]pyrimidine (1.29 g, 4.61 mmol, 1.2 eq) in MeCN (5 mL) were added BSA (938.04 mg, 4.61 mmol, 1.14 mL, 1.2 eq). The mixture was stirred at 20° C. for 0.5 hr. Then a solution of (2S,3R,4R,5R)-5-methyltetrahydrofuran-2,3,4-triyl triacetate (1 g, 3.84 mmol, 1 eq) in MeCN (5 mL) was added to the mixture. TMSOTf (1.71 g, 7.69 mmol, 1.39 mL, 2 eq) was then added to the mixture dropwise at 0° C. The mixture was stirred at 20° C. for 12 hr. The reaction mixture was poured into a saturated aqueous solution of NaHCO3 (50 mL) slowly and stirred at 0° C. for 10 min. The mixture was extracted with EtOAc (20 mL×2). The combined organic phase was washed with brine (50 mL), dried with anhydrous Na2SO4, filtered and concentrated under vacuum. The residue was purified by column chromatography (SiO2, Petroleum ether/Ethyl acetate=100/1 to 3/1) to afford Intermediate Compound 38 (600 mg, 32% yield) as a yellow oil.

LCMS: Rt=0.672 min; (ESI positive ion) m/z: 479.8 (M+H)+(calculated: 479.97).

### Intermediate Compound 39:

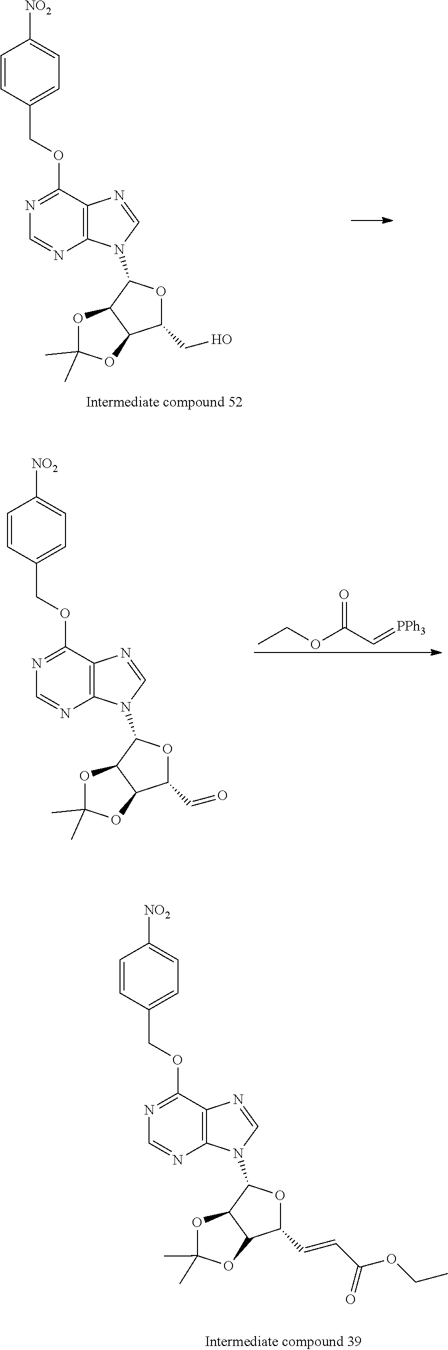

To a solution of Intermediate Compound 52 (880 mg, 1.98 mmol, 1 eq) in MeCN (15 mL) was added IBX (2-Iodoxybenzoic acid) (1.67 g, 5.95 mmol, 3 eq). The mixture was stirred at 100° C. for 45 min. The reaction mixture was filtered and washed with DCM (30 mL×2). The combined organic layers were concentrated under vacuum to afford the corresponding aldehyde (880 mg, 100% yield) (crude) as a yellow oil, which was used directly in the next step.

A mixture of the above aldehyde compound (880 mg, 1.99 mmol, 1 eq) and ethyl 2-(triphenyl-1⁵-phosphaneylidene)acetate (1.04 g, 2.99 mmol, 1.5 eq) in toluene (40 mL) was degassed and purged with N2 3 times. The mixture was stirred at 25° C. for 16 hr under N2 atmosphere. The reaction mixture was concentrated under vacuum. The resultant residue was purified by column chromatography (SiO2, Petroleum ether/Ethyl acetate=1/0 to 1/1; Petroleum ether:Ethyl acetate=3/1 Rf=0.6) followed by reversed-phase HPLC (0.1% FA condition) and lyophilization. The product was then further purified by Prep-HPLC (column: Waters Xbridge 150×25 mm×5 μm; mobile phase: [water(NH4HCO3)-ACN]; B %: 46%-76%, 9 min) to afford Intermediate Compound 39 (190 mg, 76% yield) as a yellow solid.

1H NMR (400 MHz, CDCl₃) δ=8.53 (s, 1H), 8.27-8.21 (m, 2H), 8.05 (s, 1H), 7.71 (d, J=8.8 Hz, 2H), 6.99-6.93 (m, 1H), 6.19 (d, J=2.0 Hz, 1H), 5.88-5.83 (m, 1H), 5.78 (s, 2H), 5.55-5.52 (m, 1H), 5.16-5.13 (m, 1H), 4.85-4.82 (m, 1H), 4.12 (q, J=7.2 Hz, 2H), 1.65 (s, 3H), 1.41 (s, 3H), 1.22 (t, J=7.2 Hz, 3H).

### Intermediate Compound 40:

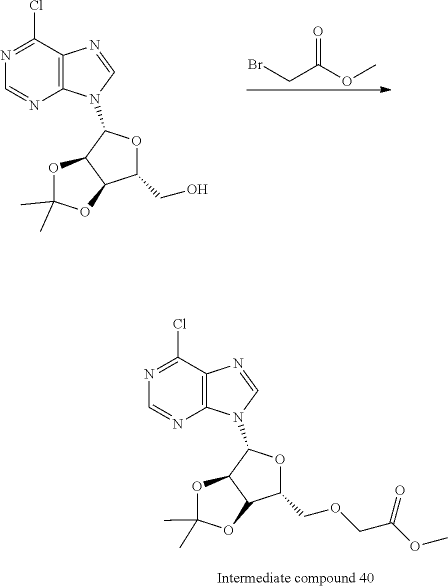

To a solution of ((3aR,4R,6R,6aR)-6-(6-chloro-9H-purin-9-yl)-2,2-dimethyltetrahydrofuro[3,4-d][1,3]dioxol-4-yl)methanol (1 g, 3.06 mmol, 1 eq) and methyl 2-bromoacetate (702.29 mg, 4.59 mmol, 433.51 μL, 1.5 eq) in DMF (10 mL) was added sodium hydride (183.64 mg, 4.59 mmol, 60% purity, 1.5 eq) at 0° C. The mixture was stirred at 0° C. for 1 hr. The reaction mixture was poured into ice-cold HCl (0.5 N 100 mL) and stirred for 1 min. The aqueous phase was extracted with ethyl acetate (50 mL×2). The combined organic phase was washed with brine (100 mL), dried with anhydrous Na2SO4, filtered and concentrated under vacuum. The resultant residue was purified by column chromatography (SiO2, Petroleum ether/Ethyl acetate=100/1 to 3/1) to afford Intermediate Compound 40 (1 g, 82% yield) as a yellow oil.

### Intermediate Compound 41:

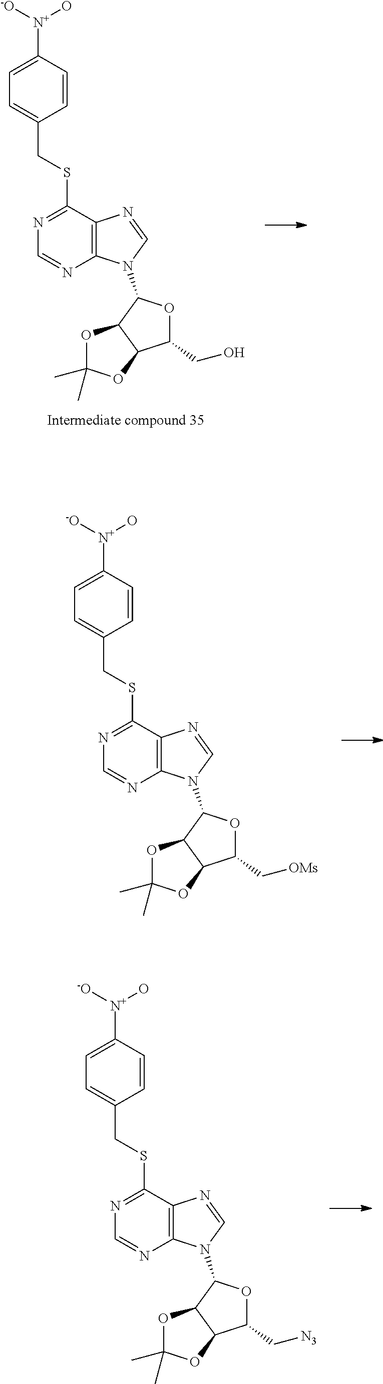

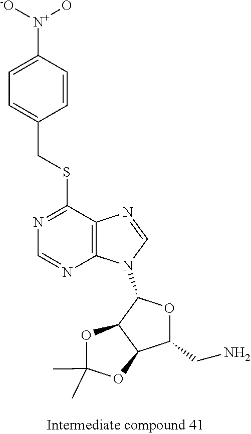

To a solution of Intermediate Compound 35 (200 mg, 435.28 μmol, 1 eq) in DCM (4 mL) was added DIEA (168.77 mg, 1.31 mmol, 227.45 μL, 3 eq) and MsCl (74.79 mg, 652.92 μmol, 50.54 μL, 1.5 eq) at 0° C. The mixture was stirred at 20° C. for 2 hours. The reaction mixture was filtered to afford the corresponding mesylated compound (230 mg, 98% yield) as a yellow oil.

LCMS: Rt=0.943 min; (ESI positive ion) m/z: 538.3 (M+H)+(calculated: 538.10).

To a solution of the above mesylated compound (180 mg, 334.84 μmol, 1 eq) in DMF (10 mL) was added sodium azide (108.84 mg, 1.67 mmol, 5 eq). The mixture was stirred at 80° C. for 16 hours. The reaction mixture was added to ice water (80 mL) and was extracted with ethyl acetate (80 mL×2). The combined organic phase was washed with brine (80 ml), dried with anhydrous Na2SO4, filtered and concentrated under vacuum to afford the corresponding azide compound (130 mg, 80% yield) as a yellow oil.

LCMS: Rt=0.980 min; (ESI positive ion) m/z: 484.9 (M+H)+(calculated: 485.13).

A mixture of the above azide compound (130 mg, 268.32 μmol, 1 eq) and triphenylphosphine (105.57 mg, 402.49 μmol, 1.5 eq) in THE (2 mL) and water (0.4 mL) was stirred at 50° C. for 1 hour. The reaction mixture was filtered and concentrated under reduced pressure to give a residue. The residue was purified by Prep-HPLC (column: Waters Xbridge 150×25 mm×5 μm; mobile phase: [water (0.05% ammonia hydroxide v/v)-ACN]; B %: 30%-57%, 9 min) to afford the Intermediate Compound 41 (60 mg, 49% yield) as a white solid.

LCMS: Rt=0.915 min; (ESI positive ion) m/z: 459.1 (M+H)+(calculated: 459.14).

### Intermediate Compound 42:

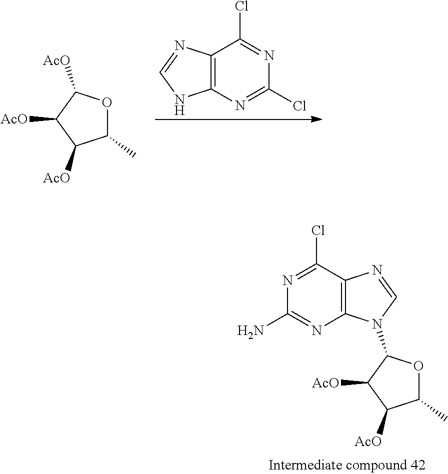 [SMILES: CC(=O)O[C@@H]1O[C@H](C)[C@@H](OC(C)=O)[C@H]1OC(C)=O ; Clc1nc(Cl)c2nc[nH]c2n1 ; CC(=O)O[C@@H]1[C@H](OC(C)=O)[C@@H](C)O[C@H]1n1cnc2c(Cl)nc(N)nc21]

Intermediate Compound 42 (2.8 g, 39% yield) was synthesized using 6-chloro-9H-purin-2-amine and following the procedure described for Intermediate Compound 27.

LCMS: Rt=0.763 min; (ESI positive ion) m/z: 370.0 (M+H)+(calculated: 370.08).

1H NMR (400 MHz, CDCl₃) δ=7.85 (s, 1H), 6.01-5.96 (m, 1H), 5.95-5.91 (m, 1H), 5.45 (t, J=5.4 Hz, 1H), 5.17 (br s, 2H), 4.30 (quin, J=6.1 Hz, 1H), 2.14 (s, 3H), 2.10 (s, 3H), 1.48 (d, J=6.4 Hz, 3H).

### Intermediate Compound 43:

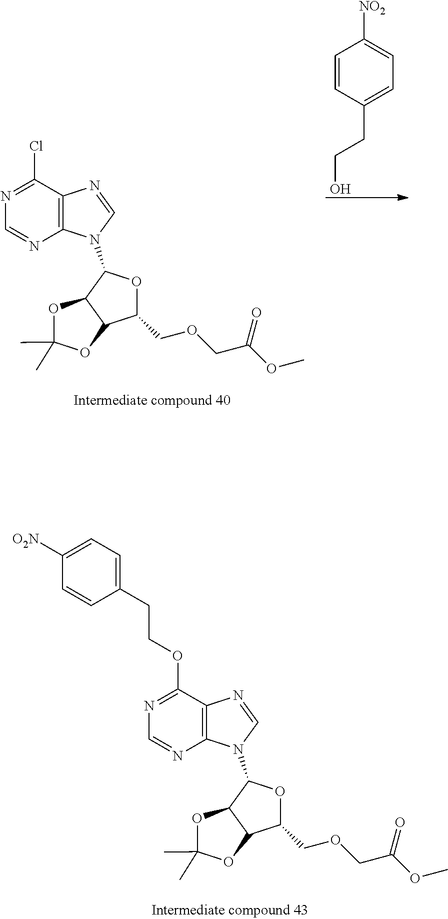

To a mixture of Intermediate Compound 40 (800 mg, 2.01 mmol, 1 eq) and 2-(4-nitrophenyl)ethan-1-ol (402.40 mg, 2.41 mmol, 1.2 eq) in toluene (10 mL) was added Cs2CO3 (1.63 g, 5.02 mmol, 2.5 eq), Xantphos (232.15 mg, 401.21 μmol, 0.2 eq), and Pd2(dba)3 (183.70 mg, 200.60 μmol, 0.1 eq). The mixture was stirred at 80° C. for 0.5 hr. The mixture was then filtered and concentrated under vacuum. The resultant residue was purified by column chromatography (SiO2, Petroleum ether/Ethyl acetate=100/1 to 1/1) to afford Intermediate Compound 43 (400 mg, 38% yield) as a yellow oil.

LCMS: Rt=0.919 min; (ESI positive ion) m/z: 530.0 (M+H)+(calculated: 530.18).

### Intermediate Compound 44:

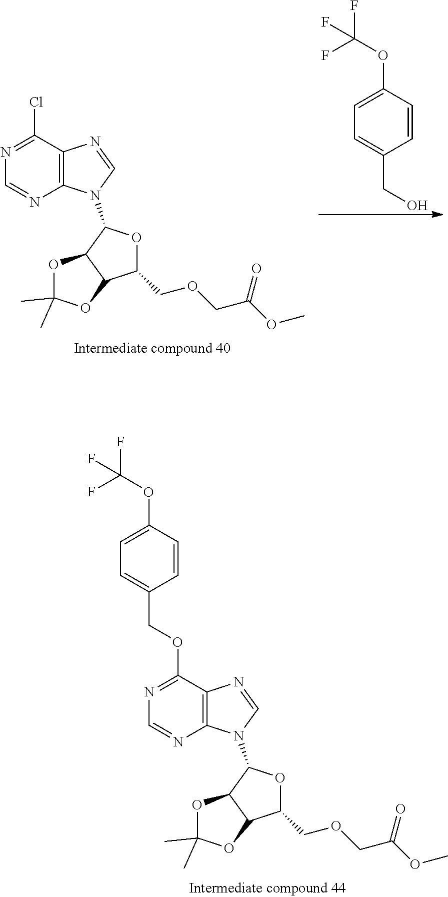

A mixture of Intermediate Compound 40 (500 mg, 1.25 mmol, 1 eq), (4-(trifluoromethoxy)phenyl)methanol (361.34 mg, 1.88 mmol, 271.68 μL, 1.5 eq), Cs2CO3 (1.02 g, 3.13 mmol, 2.5 eq), Pd2(dba)3 (114.81 mg, 125.38 μmol, 0.1 eq) and Xantphos (145.09 mg, 250.75 μmol, 0.2 eq) in toluene (10 mL) was degassed and purged with N2 3 times. The mixture was then stirred at 80° C. for 0.5 hr under N2 atmosphere. The mixture was filtered and concentrated under vacuum. The resultant residue was purified by column chromatography (SiO2, Petroleum ether/Ethyl acetate=100/1 to 1/1) to afford Intermediate Compound 44 (250 mg, 36% yield) as a yellow oil.

LCMS: Rt=0.905 min; (ESI positive ion) m/z: 555.2 (M+H)+(calculated: 555.16).

### Intermediate Compound 45:

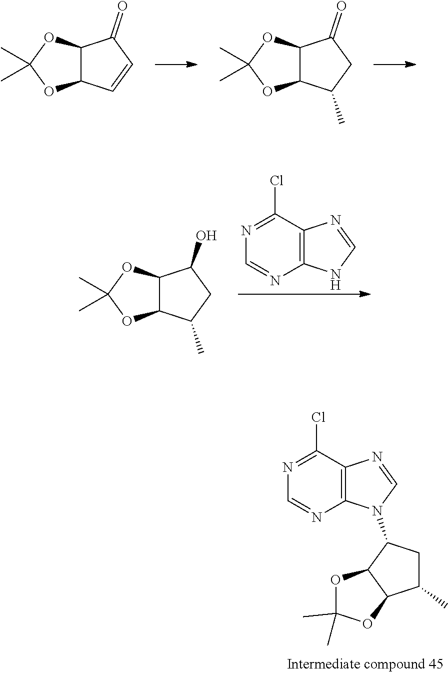

MeLi (1.6 M, 18.65 mL, 2.3 eq) was added dropwise to a mixture of CuI (2.96 g, 15.57 mmol, 1.2 eq) in THE (20 mL) at 0° C. The mixture was stirred at 0° C. for 10 min. Then a solution of (3aR,6aR)-2,2-dimethyl-3a,6a-dihydro-4H-cyclopenta[d][1,3]dioxol-4-one (2 g, 12.97 mmol, 1 eq) in THE (15 mL) was added dropwise at −78° C. The mixture was stirred at −78° C. for 20 min. The reaction mixture was quenched by addition of a saturated solution of NH4Cl (30 mL) at 0° C., and then diluted with water (30 mL) and extracted with ethyl acetate (50 mL×2). The combined organic layers were washed with brine (100 mL), dried over Na2SO4, filtered and concentrated under reduced pressure to give a residue. The residue was purified by column chromatography (SiO2, Petroleum ether/Ethyl acetate=100/0 to 3/1) to afford the methylated compound (1.6 g, 72% yield) as a yellow oil.

1H NMR (400 MHz, CDCl₃-d) δ=4.50 (d, J=5.6 Hz, 1H), 4.23 (d, J=5.6 Hz, 1H), 2.81 (dd, J=8.4, 18.4 Hz, 1H), 2.53 (quin, J=8.0 Hz, 1H), 1.97 (d, J=18.4 Hz, 1H), 1.4 (s, 3H), 1.35 (s, 3H), 1.05 (d, J=7.6 Hz, 3H).

To a solution of the above methylated compound (600 mg, 3.53 mmol, 1 eq) and 3A molecular sieves (600 mg, 3.53 mmol, 1 eq) in DCM (10 mL) was added dropwise DIBAL-H (1 M, 7.05 mL, 2 eq) at −78° C. The mixture was stirred at −78° C. for 4 hr. The reaction mixture was quenched by addition of MeOH (10 mL) at 0° C., and then diluted with water (30 mL) and extracted with DCM (30 mL×2). The combined organic layers were washed with brine (80 mL), dried over Na2SO4, filtered and concentrated under reduced pressure to give a residue. The residue was purified by column chromatography (SiO2, Petroleum ether/Ethyl acetate=100/0 to 3/1) to afford the corresponding alcohol compound (460 mg, 76% yield) as a yellow oil.

1H NMR (400 MHz, CDCl₃) δ=4.50 (t, J=6.0 Hz, 1H), 4.28 (d, J=6.0 Hz, 1H), 4.15-4.09 (m, 1H), 2.22-2.10 (m, 2H), 1.85 (ddd, J=7.2, 8.8, 12.8 Hz, 1H), 1.65 (ddd, J=3.6, 6.0, 12.8 Hz, 1H), 1.50 (s, 3H), 1.34 (s, 3H), 0.93 (d, J=8.0 Hz, 3H).

To a solution of the above alcohol compound (200 mg, 1.16 mmol, 1 eq) and 6-chloro-9H-purine (197.44 mg, 1.28 mmol, 1.1 eq) in toluene (5 mL) was added triphenylphosphine (913.78 mg, 3.48 mmol, 3 eq). To the mixture was added DIAD (704.47 mg, 3.48 mmol, 677.38 μL, 3 eq) at 0° C. The mixture was stirred for 0.5 hr. The mixture was warmed and stirred at 80° C. for 12 hr. The reaction mixture was concentrated under reduced pressure to remove solvent. The resultant residue was purified by column chromatography (SiO2, Petroleum ether/Ethyl acetate=100/0 to 1/1) to afford Intermediate Compound 45 (300 mg, crude) as a red oil.

LCMS: Rt=0.808 min; (ESI positive ion) m/z: 309.1 (M+H)+(calculated:309.10). 1H NMR (400 MHz, CDCl₃) δ=8.74 (s, 1H), 8.18 (s, 1H), 5.07 (dd, J=6.0, 7.2 Hz, 1H), 4.86-4.76 (m, 1H), 4.45 (dd, J=5.6, 7.2 Hz, 1H), 2.52-2.45 (m, 1H), 2.34-2.25 (m, 2H), 1.77 (s, 3H), 1.27 (s, 6H).

### Intermediate Compound 46:

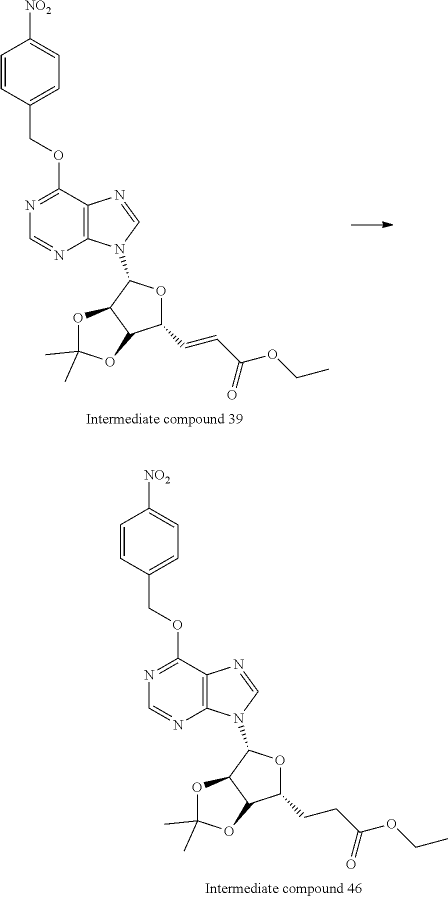

To a solution of Intermediate Compound 39 (190 mg, 371.47 μmol, 1 eq) in EtOAc (38 mL) was added Pd/C (38 mg, 10% purity) under N2 atmosphere. The suspension was degassed and purged with H2 3 times. The mixture was stirred under H2 (15 psi) at 25° C. for 1 hr. The reaction mixture was filtered, and the filtrate was concentrated under vacuum. The residue was purified by Prep-HPLC (column: Waters Xbridge 150×25 mm×5 μm; mobile phase: [water (NH4HCO3)-ACN]; B %: 50%-80%, 10 min) and lyophilized to afford Intermediate Compound 46 (70 mg, 37% yield) as colorless oil.

1HNMR (400 MHz, CDCl₃) δ=8.57-8.50 (m, 1H), 8.24 (br d, J=8.6 Hz, 2H), 8.04 (s, 1H), 7.71 (br d, J=8.4 Hz, 2H), 6.09 (br s, 1H), 5.78 (s, 2H), 5.50-5.41 (m, 2H), 4.95-4.83 (m, 1H), 4.28-4.19 (m, 2H), 4.09 (td, J=6.6, 13.3 Hz, 2H), 2.46-2.33 (m, 3H), 2.15-1.98 (m, 4H), 1.66-1.64 (m, 3H), 1.48-1.36 (m, 3H), 1.26-1.18 (m, 3H).

### Intermediate Compound 47:

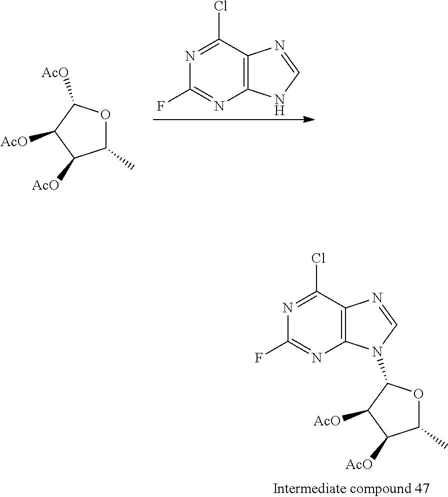 [SMILES: CC(=O)O[C@@H]1O[C@H](C)[C@@H](OC(C)=O)[C@H]1OC(C)=O ; Fc1nc(Cl)c2nc[nH]c2n1 ; CC(=O)O[C@@H]1[C@H](OC(C)=O)[C@@H](C)O[C@H]1n1cnc2c(Cl)nc(F)nc21]

Intermediate Compound 47 (400 mg, 28% yield) was synthesized from 6-chloro-2-fluoro-9H-purine following the protocol described for Intermediate Compound 27.

LCMS: Rt=0.847 min; (ESI positive ion) m/z: 373.0 (M+H)+(calculated:373.06). 1H NMR (400 MHz, CDCl₃) δ=8.20 (s, 1H), 6.08 (d, J=5.3 Hz, 1H), 5.83 (t, J=5.4 Hz, 1H), 5.32 (t, J=5.3 Hz, 1H), 4.41-4.34 (m, 1H), 2.17 (s, 3H), 2.09 (s, 3H), 1.54 (d, J=6.5 Hz, 3H)

### Intermediate Compound 48:

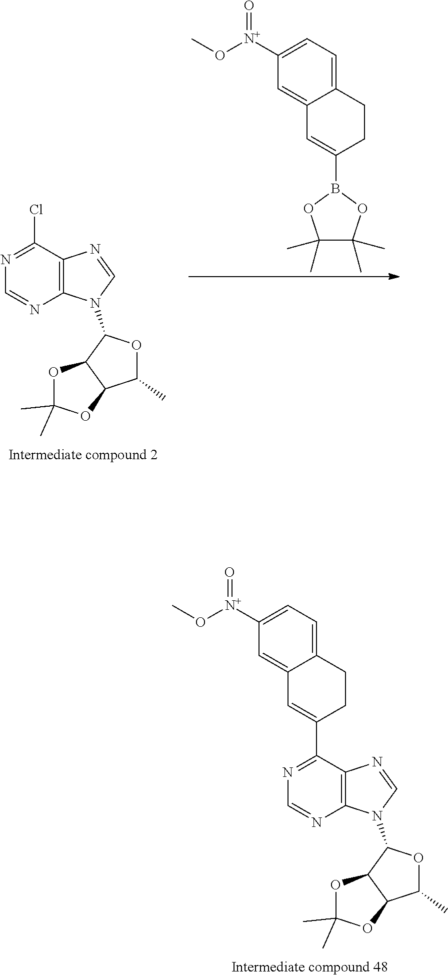

To a solution of 4,4,5,5-tetramethyl-2-(7-nitro-3,4-dihydronaphthalen-2-yl)-1,3,2-dioxaborolane (200 mg, 664.13 μmol, 1 eq) and Intermediate Compound 2 (206.37 mg, 664.13 mol, 1 eq) in dioxane (20 mL) and water (2 mL) was added Na2CO3 (175.98 mg, 1.66 mmol, 2.5 eq) and Pd(dppf)Cl2·CH2Cl2 (54.24 mg, 66.41 μmol, 0.1 eq) under N2. The mixture was stirred at 80° C. for 16 hr. The reaction mixture was concentrated under reduced pressure to give a residue. The residue was purified by column chromatography (SiO2, Petroleum ether/Ethyl acetate=1/0 to 2/1) to afford Intermediate Compound 48 (169 mg, 57% yield) as a yellow solid.

1H NMR (400 MHz, CDCl₃) δ=9.01 (s, 1H), 8.62 (s, 1H), 8.27-8.19 (m, 2H), 8.12-8.08 (m, 1H), 7.37 (d, J=8.4 Hz, 1H), 6.16 (d, J=2.4 Hz, 1H), 5.58-5.55 (m, 1H), 4.84-4.81 (m, 1H), 4.51-4.40 (m, 1H), 3.26-3.08 (m, 4H), 1.65 (s, 3H), 1.43-1.39 (m, 6H)

### Intermediate Compound 49:

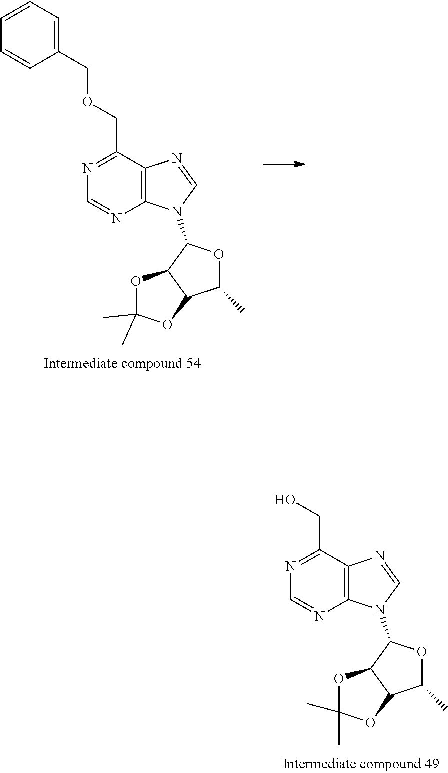

A mixture of Intermediate Compound 54 (450 mg, 1.14 mmol, 1 eq), Pd/C (50 mg, 10% purity) in MeOH (10 mL) was degassed and purged with H2 (15 PSI) 3 times. The mixture was stirred at 40° C. for 32 hr under H2 (15 PSI) atmosphere. The mixture was then filtered and concentrated under vacuum. The resultant residue was purified by column chromatography (SiO2, Petroleum ether/Ethyl acetate=100/1 to 0/1) to afford Intermediate Compound 49 (210 mg, 60% yield) as a yellow oil.

LCMS: Rt=0.807 min; (ESI positive ion) m/z: 307.0 (M+H)+(calculated: 307.13).

### Intermediate Compound 50:

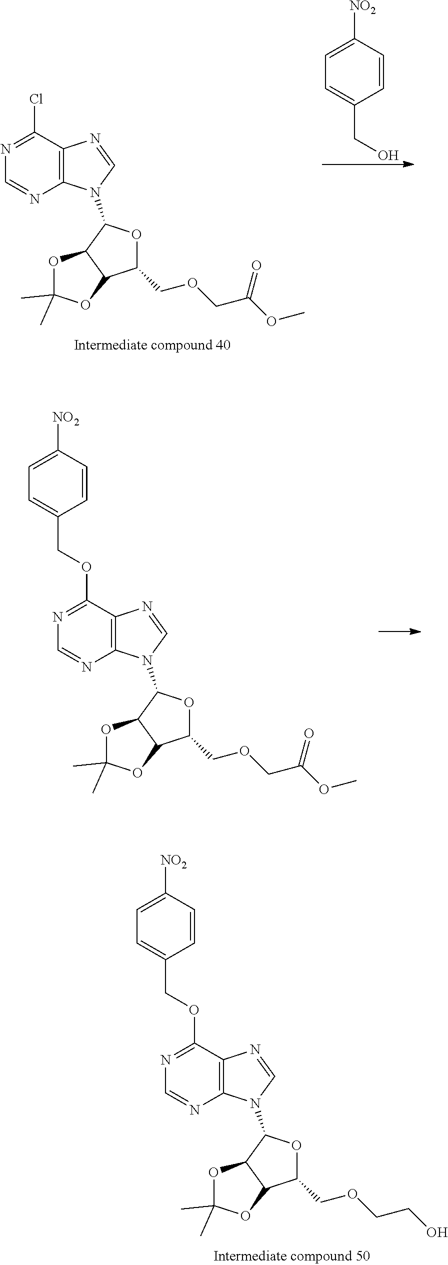

A mixture of Intermediate Compound 40 (3.1 g, 7.77 mmol, 1 eq), (4-nitrophenyl)methanol (1.43 g, 9.33 mmol, 1.2 eq), Pd2(dba)3 (711.82 mg, 777.34 μmol, 0.1 eq), Xantphos (899.56 mg, 1.55 mmol, 0.2 eq) and Cs2CO3 (6.33 g, 19.43 mmol, 2.5 eq) in toluene (30 mL) was degassed and purged with N2 3 times. The mixture was stirred at 80° C. for 0.5 hr under N2 atmosphere. The mixture was then filtered and concentrated under vacuum. The resultant residue was purified by column chromatography (SiO2, Petroleum ether/Ethyl acetate=100/1 to 1/1) to afford 3.6 g crude product, which was purified by reversed-phase HPLC (0.1% FA condition) to afford the corresponding ether compound (2.2 g, 51% yield) as a yellow oil. LCMS: Rt=0.433 min; (ESI positive ion) m/z: 516.1 (M+H)+(calculated:516.17). To a solution of the above ether compound (1 g, 1.94 mmol, 1 eq) in THE (15 mL) was added LiBH4 (2 M, 1.07 mL, 1.1 eq) at 0° C. under N2. The mixture was stirred at 0° C. for 1 hr. The mixture was added to ice-water (100 mL) dropwise at 0° C. and stirred for 5 min. The mixture was extracted with EtOAc (50 mL×2). The combined organic phase was washed with brine (100 mL), dried with anhydrous Na2SO4, filtered and concentrated under vacuum. The resultant residue was purified by column chromatography (SiO2, Petroleum ether/Ethyl acetate=100/1 to 0/1) to afford Intermediate Compound 50 (300 mg, 615.43 μmol, 31.72% yield) as a yellow oil.

LCMS: Rt=0.470 min; (ESI positive ion) m/z: 488.2 (M+H)+(calculated:488.17).

### Intermediate Compound 51:

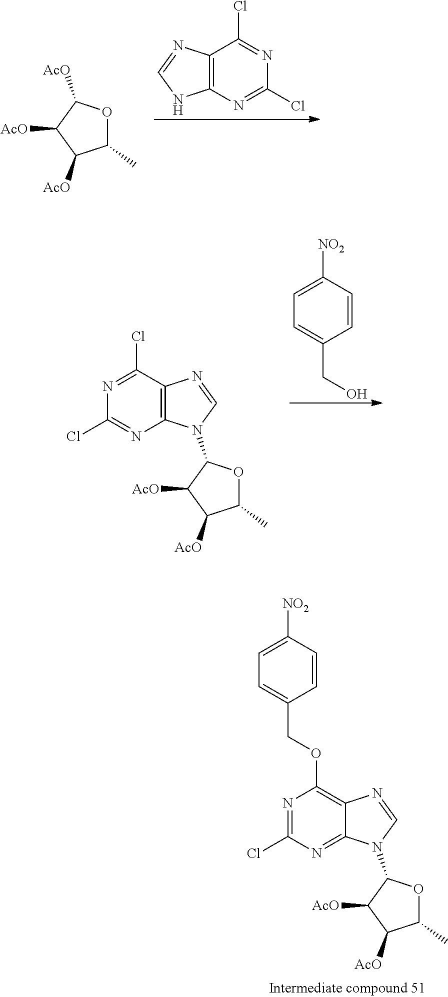 [SMILES: CC(=O)O[C@@H]1O[C@H](C)[C@@H](OC(C)=O)[C@H]1OC(C)=O ; Clc1nc(Cl)c2nc[nH]c2n1 ; CC(=O)O[C@@H]1[C@H](OC(C)=O)[C@@H](C)O[C@H]1n1cnc2c(Cl)nc(Cl)nc21 ; O=[N+]([O-])c1ccc(CO)cc1 ; CC(=O)O[C@@H]1[C@H](OC(C)=O)[C@@H](C)O[C@H]1n1cnc2c(OCc3ccc([N+](=O)[O-])cc3)nc(Cl)nc21]

To a solution of 2,6-dichloro-9H-purine (1 g, 3.84 mmol, 1 eq) and (2S,3R,4R,5R)-5-methyltetrahydrofuran-2,3,4-triyl triacetate (871.51 mg, 4.61 mmol, 1.2 eq) in 1,2-dichloroethane (15 mL) was added BSA (1.56 g, 7.69 mmol, 1.90 mL, 2 eq). The mixture was stirred at 80° C. for 0.5 hr until the solution became clear. After 0.5 hr, the mixture was cooled to room temperature, and then TMSOTf (1.11 g, 5.00 mmol, 902.65 μL, 1.3 eq) was added to the mixture dropwise. The mixture was stirred at 80° C. for 12 hr. The reaction mixture was then poured into a saturated aqueous solution of NaHCO3 (30 mL) and extracted with DCM (20 mL×3). The organic layers were combined and washed with brine, dried by Na2SO4, and concentrated to give a residue. The residue was purified by flash silica gel chromatography (ISCO®; 12 g SepaFlash® Silica Flash Column, Eluent of 35-45% Ethyl acetate/Petroleum ether gradient @40 mL/min). The purified solution was concentrated to afford the corresponding conjugated intermediate compound (1.2 g, 80% yield) as a colorless gum.

LCMS: Rt=0.874 min; (ESI positive ion) m/z: 389.0 (M+H)+(calculated:389.03). 1H NMR (400 MHz, CDCl₃) δ=8.22 (s, 1H), 6.12 (d, J=5.3 Hz, 1H), 5.82 (t, J=5.5 Hz, 1H), 5.37-5.32 (m, 1H), 4.43-4.34 (m, 1H), 2.17 (s, 3H), 2.10 (s, 3H), 1.55 (d, J=6.6 Hz, 3H)

A mixture of the above conjugated intermediate compound (1.5 g, 3.85 mmol, 1 eq), (4-nitrophenyl)methanol (590.21 mg, 3.85 mmol, 1 eq), Cs2CO3 (2.51 g, 7.71 mmol, 2 eq), Xantphos (446.02 mg, 770.83 μmol, 0.2 eq) and Pd2(dba)3 (352.93 mg, 385.42 μmol, 0.1 eq) in toluene (30 mL) was degassed and purged with N2 3 times. The mixture was stirred at 80° C. for 1 hr under N2 atmosphere. The reaction mixture was concentrated to give a residue. The residue was purified by column chromatography (SiO2, Petroleum ether/Ethyl acetate=1/0 to 0/1) to afford Intermediate Compound 51 (1.03 g, 52% yield) as a yellow solid.

LCMS: Rt=0.985 min; (ESI positive ion) m/z: 506.0 (M+H)+(calculated:506.10).

### Intermediate Compound 52:

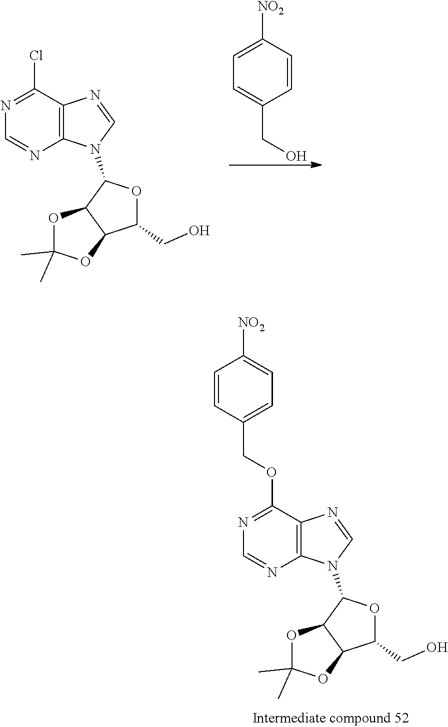 [SMILES: CC1(C)O[C@@H]2[C@H](O1)[C@@H](CO)O[C@H]2n1cnc2c(Cl)ncnc21 ; O=[N+]([O-])c1ccc(CO)cc1 ; CC1(C)O[C@@H]2[C@H](O1)[C@@H](CO)O[C@H]2n1cnc2c(OCc3ccc([N+](=O)[O-])cc3)ncnc21]

To a solution of ((3aR,4R,6R,6aR)-6-(6-chloro-9H-purin-9-yl)-2,2-dimethyltetrahydrofuro[3,4-d][1,3]dioxol-4-yl)methanol (5 g, 15.30 mmol, 1 eq) and (4-nitrophenyl)methanol (2.81 g, 18.36 mmol, 1.2 eq) in toluene (80 mL) was added Cs2CO3 (12.46 g, 38.26 mmol, 2.5 eq) and Xantphos (1.77 g, 3.06 mmol, 0.2 eq) and Pd2(dba)3 (1.40 g, 1.53 mmol, 0.1 eq). The mixture was stirred at 80° C. for 0.5 hr. The reaction mixture was filtered and concentrated under reduced pressure to give a residue, which was purified by column chromatography (SiO2, Petroleum ether/Ethyl acetate=100/1 to 1/1), TLC (Petroleum ether/Ethyl acetate=1:1, rf=0.4) to afford Intermediate Compound 52 (2.8 g, 40% yield) as a yellow oil.

1H NMR (400 MHz, DMSO-d6) δ=8.64-8.57 (m, 2H), 8.27 (d, J=8.8 Hz, 2H), 7.77 (d, J=8.8 Hz, 2H), 6.24 (d, J=2.7 Hz, 1H), 5.80 (s, 2H), 5.39 (dd, J=2.7, 6.1 Hz, 1H), 5.13-4.97 (m, 2H), 4.26 (dt, J=2.5, 4.8 Hz, 1H), 3.60-3.50 (m, 2H), 1.55 (s, 3H), 1.33 (s, 3H).

### Intermediate Compound 53:

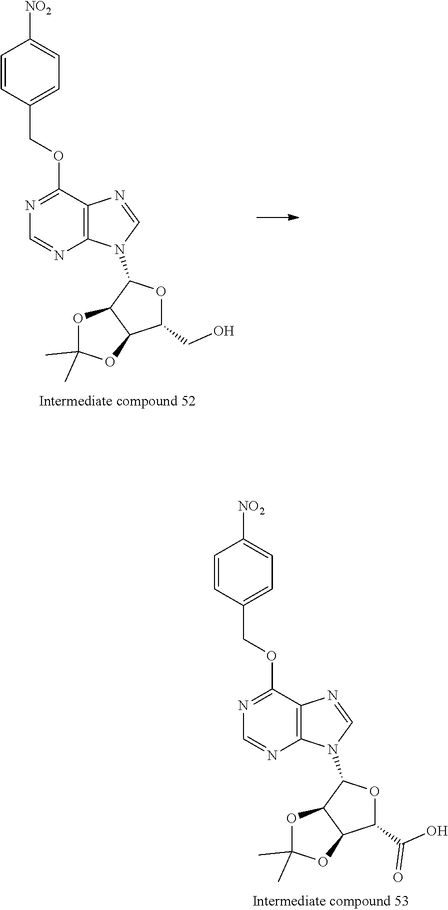 [SMILES: CC1(C)O[C@@H]2[C@H](O1)[C@@H](CO)O[C@H]2n1cnc2c(OCc3ccc([N+](=O)[O-])cc3)ncnc21 ; CC1(C)O[C@@H]2[C@H](O1)[C@@H](C(=O)O)O[C@H]2n1cnc2c(OCc3ccc([N+](=O)[O-])cc3)ncnc21]

To a solution of Intermediate Compound 52 (500 mg, 1.13 mmol, 1 eq) in water (1.5 mL), MeCN (1 mL) and CCl4 (1 mL) was added sodium periodate (988.88 mg, 4.62 mmol, 256.19 μL, 4.1 eq) and trichlororutheniμm; trihydrate (64.87 mg, 248.08 μmol, 0.22 eq). The mixture was stirred at 20° C. for 12 hr. The reaction mixture was filtered and the filter cake was washed with EtOAc (30 ml×3). The filtrate was then dried by NaSO4 and concentrated under reduced pressure to give a residue. The residue was purified by column chromatography (SiO2, Petroleum ether/Ethyl acetate=100/1 to 1/1) to afford Intermediate Compound 53 (100 mg, 19% yield) as a white solid.

1H NMR (400 MHz, CDCl₃) δ=8.45-8.26 (m, 2H), 8.10 (br d, J=8.1 Hz, 2H), 7.65-7.55 (m, 2H), 6.13-6.06 (m, 1H), 5.60-5.49 (m, 2H), 5.17-4.96 (m, 2H), 4.52-4.43 (m, 1H), 3.50 (s, 1H), 1.41 (br s, 3H), 1.20 (br s, 3H).

### Intermediate Compound 54:

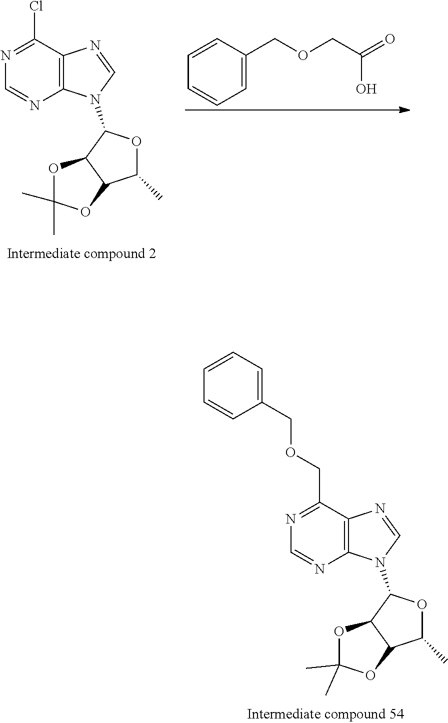

A mixture of Intermediate Compound 2 (723 mg, 2.33 mmol, 1 eq), 2-(benzyloxy)acetic acid (502.63 mg, 3.02 mmol, 433.31 μL, 1.3 eq), [4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate (26.10 mg, 23.27 μmol, 0.01 eq), bis(1,1-dimethylethyl)-2,2′-bipyridine]nickel (II) dichloride (46.30 mg, 116.34 μmol, 0.05 eq) and Cs2CO3 (1.14 g, 3.49 mmol, 1.5 eq) in DMA (15 mL) was degassed and purged with N2 3 times. The mixture was then irradiated with two 34 W blue LED lamps (at approximately 7 cm away from the light source) to keep the reaction temperature at 25° C. for 14 hr. The mixture was poured into H2O (50 mL) and extracted with EtOAc (20 mL×2). The combined organic phase was washed with brine (50 mL), dried with anhydrous Na2SO4, filtered, and concentrated under vacuum. The resultant residue was purified by column chromatography (SiO2, Petroleum ether/Ethyl acetate=100/1 to 1/1) to afford Intermediate Compound 54 (450 mg, 45% yield) as a yellow oil.

LCMS: Rt=0.846 min; (ESI positive ion) m/z: 396.9 (M+H)+(calculated: 397.18).

1H NMR (400 MHz, CDCl₃) δ=9.05 (s, 1H), 8.22 (s, 1H), 7.44 (d, J=7.2 Hz, 2H), 7.38-7.32 (m, 2H), 7.31-7.28 (m, 1H), 6.12 (d, J=2.4 Hz, 1H), 5.54 (dd, J=2.2, 6.4 Hz, 1H), 5.10 (s, 2H), 4.83-4.79 (m, 3H), 4.44 (dq, J=3.6, 6.6 Hz, 1H), 1.64 (s, 3H), 1.41-1.37 (m, 6H).

### Intermediate Compound 55:

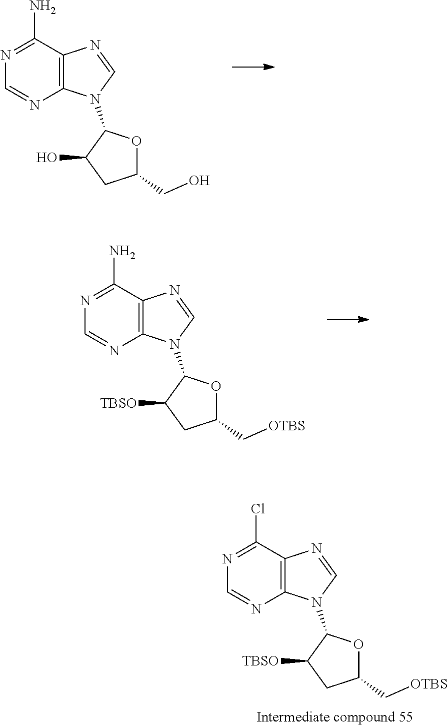 [SMILES: Nc1ncnc2c1ncn2[C@@H]1O[C@H](CO)C[C@H]1O ; CC(C)(C)[Si](C)(C)OC[C@@H]1C[C@@H](O[Si](C)(C)C(C)(C)C)[C@H](n2cnc3c(N)ncnc32)O1 ; CC(C)(C)[Si](C)(C)OC[C@@H]1C[C@@H](O[Si](C)(C)C(C)(C)C)[C@H](n2cnc3c(Cl)ncnc32)O1]

To a mixture of (2R,3R,5S)-2-(6-amino-9H-purin-9-yl)-5-(hydroxymethyl)tetrahydrofuran-3-ol (1.6 g, 6.37 mmol, 1 eq) and imidazole (2.60 g, 38.21 mmol, 6 eq) in DMF (42 mL) was added a solution of TBSCI (2.88 g, 19.11 mmol, 2.34 mL, 3 eq) in DMF (30 mL) dropwise under nitrogen atmosphere at 0° C. The mixture was slowly warmed to 20° C. and stirred for 16 hr. The reaction mixture was diluted with DCM (100 mL) and washed with a saturated aqueous solution of NH4Cl (150 mL×3), brine, and dried by Na2SO4. The solution was concentrated to afford the corresponding protected alcohol (3.58 g, crude) as a white solid.

LCMS: Rt=1.023 min; (ESI positive ion) m/z: 480.2 (M+H)+(calculated: 480.27).

1H NMR (400 MHz, CDCl₃) δ=8.34 (d, J=11.0 Hz, 2H), 6.13-5.92 (m, 3H), 4.65-4.60 (m, 1H), 4.56 (dt, J=2.8, 6.2 Hz, 1H), 4.16-4.10 (m, 1H), 3.78 (dd, J=2.7, 11.6 Hz, 1H), 2.32-2.21 (m, 1H), 1.86 (ddd, J=2.1, 5.6, 12.9 Hz, 1H), 0.96-0.91 (m, 12H), 0.90 (s, 6H), 0.14-0.12 (m, 6H), 0.11-0.07 (m, 6H).

To a solution of the above protected alcohol (3.78 g, 7.88 mmol, 1 eq) in DCM (75 mL) was added TMSCI (7.70 g, 70.91 mmol, 9.00 mL, 9 eq) dropwise at 0° C., followed by a solution of tert-butyl nitrite (4.87 g, 47.27 mmol, 5.62 mL, 6 eq) in DCM (75 mL). After stirring for 30 min, the solution was warmed to 20° C. stirred for 12 hr. The reaction mixture was slowly poured into a saturated aqueous solution of NaHCO3 (200 mL) under ice-water bath cooling. The aqueous phase was extracted with DCM (100 mL×2) and the combined organic phases were washed with brine, dried over anhydrous Na2SO4 and concentrated to give a residue. The residue was purified by flash silica gel chromatography (ISCO®; 40 g SepaFlash® Silica Flash Column, Eluent of 0-11% Ethyl acetate/Petroleum ether gradient @100 mL/min) to afford Intermediate Compound 55 (1.48 g, 38% yield) as a white solid.

LCMS: Rt=1.239 min; (ESI positive ion) m/z: 499.2 (M+H)+(calculated: 499.22).

1H NMR (400 MHz, CDCl₃) δ=8.73 (d, J=7.8 Hz, 2H), 6.09 (s, 1H), 4.67-4.57 (m, 2H), 4.18 (dd, J=2.2, 11.7 Hz, 1H), 3.79 (dd, J=2.4, 11.7 Hz, 1H), 2.27 (ddd, J=4.8, 9.8, 13.1 Hz, 1H), 1.88 (ddd, J=2.1, 5.5, 13.0 Hz, 1H), 0.95 (s, 9H), 0.93-0.91 (m, 9H), 0.15 (d, J=5.4 Hz, 9H), 0.11 (s, 3H).

### Intermediate Compound 56 and Intermediate Compound 57:

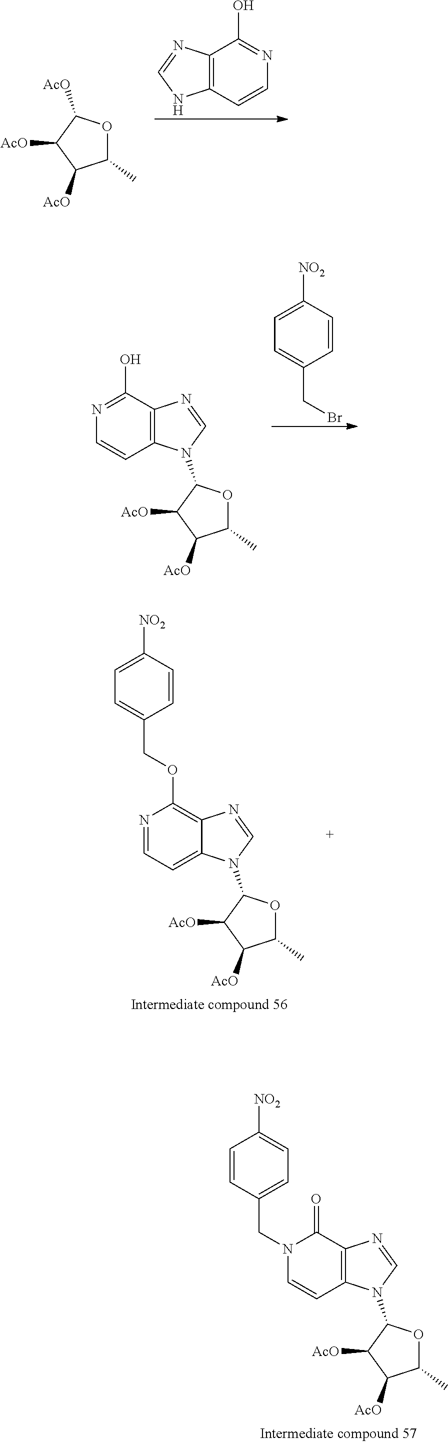 [SMILES: CC(=O)O[C@@H]1O[C@H](C)[C@@H](OC(C)=O)[C@H]1OC(C)=O ; Oc1nccc2[nH]cnc12 ; CC(=O)O[C@@H]1[C@H](OC(C)=O)[C@@H](C)O[C@H]1n1cnc2c(O)nccc21 ; O=[N+]([O-])c1ccc(CBr)cc1 ; CC(=O)O[C@@H]1[C@H](OC(C)=O)[C@@H](C)O[C@H]1n1cnc2c(OCc3ccc([N+](=O)[O-])cc3)nccc21 ; CC(=O)O[C@@H]1[C@H](OC(C)=O)[C@@H](C)O[C@H]1n1cnc2c(=O)n(Cc3ccc([N+](=O)[O-])cc3)ccc21]

A mixture of 1H-imidazo[4,5-c]pyridin-4-ol (2, 124.61 mg, 922.23 μmol, 1.2 eq, ammonium sulfate (101.55 mg, 768.52 μmol, 57.37 μL, 1 eq and pyridine (121.58 mg, 1.54 mmol, 124.06 μL, 2 eq in hexamethyldisilazane (3 mL) under N2 was heated to 140° C. in a sealed tube over a period of 2 hours. The reaction mixture was cooled and concentrated under reduced pressure. To the crude silylated pyrimidine was added (2S,3R,4R,5R)-5-methyltetrahydrofuran-2,3,4-triyl triacetate (1, 200 mg, 768.52 μmol, 1 eq and MeCN (3 mL). Then tin(IV) chloride (400.43 mg, 1.54 mmol, 179.57 μL, 2 eq was added dropwise to the mixture at 0° C. After the mixture was stirred at 20° C. for 1 hour. The reaction mixture was filtered and concentrated under reduced pressure to give a residue. The residue was purified by Prep-HPLC (column: Unisil 3-100 C18 Ultra 150×50 mm×3 μm; mobile phase: [water(FA)-ACN]; B %: 5%-35%, 10 min) to afford the conjugated intermediate compound (40 mg, 15% yield) as a yellow oil.

LCMS: Rt=0.648 min; (ESI positive ion) m/z: 336.0 (M+H)+(calculated: 336.11).

A mixture of the above conjugated intermediate compound (45 mg, 134.20 μmol, 1 eq), 1-(bromomethyl)-4-nitrobenzene (34.79 mg, 161.04 μmol, 1.2 eq) and silver carbonate (44.41 mg, 161.04 μmol, 7.30 μL, 1.2 eq) in MeCN (1 mL) was prepared. The mixture was stirred at 80° C. for 16 hours. The reaction mixture was filtered and concentrated under reduced pressure to give a residue. The residue was purified by Prep-TLC (SiO2, Petroleum ether:Ethyl acetate=1:1) to afford the 2 isomers:

Intermediate Compound 56 (10 mg, 16% yield) as a white solid.

LCMS: Rt=0.857 min; (ESI positive ion) m/z: 471.2 (M+H)+(calculated: 471.14).

1H NMR (400 MHz, CDCl₃) δ=8.12 (s, 1H), 7.53 (d, J=8.3 Hz, 2H), 7.42 (d, J=8.3 Hz, 2H), 7.14 (d, J=7.2 Hz, 1H), 6.77 (d, J=7.3 Hz, 1H), 6.66 (d, J=3.9 Hz, 1H), 5.71-5.59 (m, 1H), 5.30 (d, J=10.3 Hz, 1H), 5.21 (s, 2H), 5.11 (t, J=6.1 Hz, 1H), 4.34 (t, J=6.5 Hz, 1H), 2.09 (d, J=7.1 Hz, 6H), 1.52 (d, J=6.4 Hz, 3H).

Intermediate Compound 57 (10 mg, 21.26 μmol, 15.84% yield) as a white solid.

LCMS: Rt=0.879 min; (ESI positive ion) m/z: 471.1 (M+H)+(calculated: 471.14).

1H NMR (400 MHz, CDCl₃) δ=8.19 (d, J=8.6 Hz, 2H), 7.94 (s, 1H), 7.48 (d, J=8.7 Hz, 2H), 7.19 (d, J=7.3 Hz, 1H), 6.59 (d, J=7.3 Hz, 1H), 5.89 (d, J=5.0 Hz, 1H), 5.42 (t, J=5.2 Hz, 1H), 5.37 (d, J=5.4 Hz, 2H), 5.07 (t, J=5.4 Hz, 1H), 4.38 (quin, J=6.2 Hz, 1H), 2.13 (d, J=12.2 Hz, 6H), 1.54 (d, J=6.5 Hz, 3H).

### Synthesis of Final Compounds

### Compound 1:

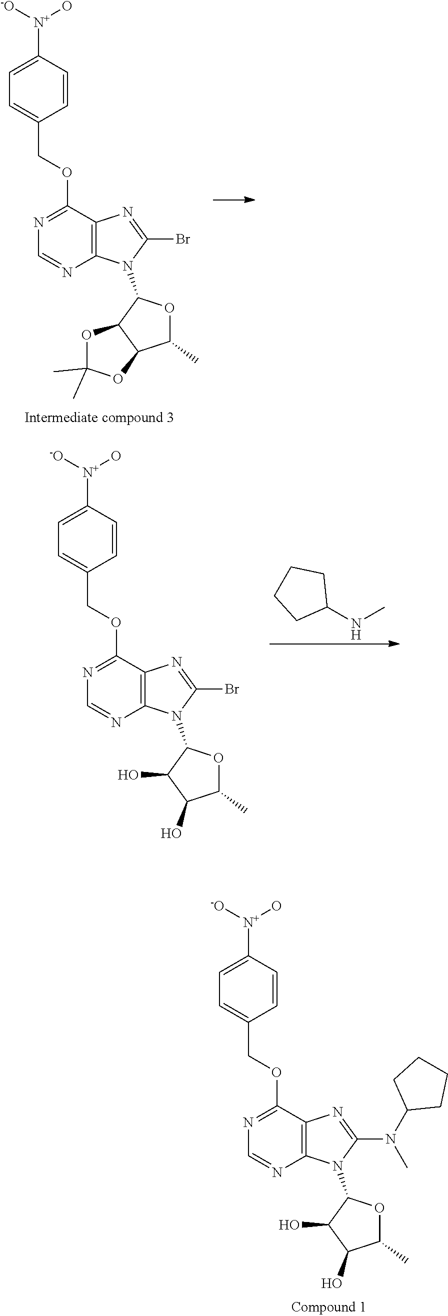

A solution of Intermediate Compound 3 (250 mg, 493.77 μmol) in formic acid (4 mL) and water (1 mL) was stirred at 0° C. for 6 hr. The reaction mixture was neutralized by addition of an aqueous solution of ammonia at 0° C. The mixture was filtered and concentrated under reduced pressure to give a residue. The residue was purified by Prep-TLC (SiO2, Petroleum ether:Ethyl acetate=1:1) to afford the corresponding deprotected diol compound (102 mg, 30% yield) as a yellow oil.

LCMS: Rt=0.492 min; (ESI positive ion) m/z: 465.90 (M+H)+(calculated: 466.03).

To a solution of the preceding diol compound (150.00 mg, 218.77 μmol, 68% purity) in DMSO (2 mL) was added N-methylcyclopentanamine (108.48 mg, 1.09 mmol) and TEA (110.69 mg, 1.09 mmol, 152.25 μL). The mixture was stirred at 110° C. for 12 hr. The reaction mixture was extracted with water (10 mL×2). The combined organic layers were washed with brine (10 mL×1), dried over anhydrous Na2SO4, filtered and concentrated under reduced pressure to give a residue. The residue was purified by Prep-HPLC (column: Phenomenex C18 150×25 mm×10 μm; mobile phase: [water (NH4HCO3)-ACN]; B %: 35%-65%, 8 min) to afford Compound 1 as a brown solid.

LCMS: Rt=0.492 min; (ESI positive ion) m/z: 485.20 (M+H)+(calculated: 485.21).

1H NMR (400 MHz, DMSO-d6) δ=8.37 (s, 1H), 8.26 (br dd, J=8.4, 4945.3 Hz, 2H), 7.74 (br d, J=8.4 Hz, 2H), 5.73 (s, 2H), 5.65 (d, J=4.8 Hz, 1H), 5.32 (d, J=5.6 Hz, 1H), 5.14-5.06 (m, 2H), 4.13 (q, J=5.4 Hz, 1H), 4.03-3.97 (m, 1H), 3.95-3.90 (m, 1H), 2.82 (s, 3H), 1.84 (br s, 2H), 1.70-1.58 (m, 4H), 1.56-1.47 (m, 2H), 1.30 (br d, J=6.4 Hz, 3H).

### Compound 2:

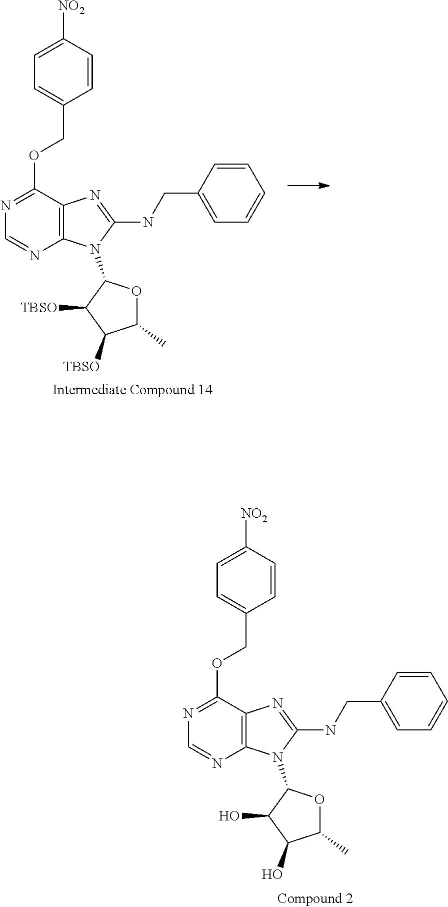 [SMILES: C[C@H]1O[C@@H](n2c(NCc3ccccc3)nc3c(OCc4ccc([N+](=O)[O-])cc4)ncnc32)[C@H](O[Si](C)(C)C(C)(C)C)[C@@H]1O[Si](C)(C)C(C)(C)C ; C[C@H]1O[C@@H](n2c(NCc3ccccc3)nc3c(OCc4ccc([N+](=O)[O-])cc4)ncnc32)[C@H](O)[C@@H]1O]

To a solution of Intermediate Compound 14 (12 mg, 16.62 μmol, 1 eq) in MeOH (0.5 mL) was added NH4F (12.31 mg, 332.42 μmol, 20 eq). The mixture was stirred at 65° C. for 12 hr. The reaction mixture was then concentrated under reduced pressure to give a residue. The residue was purified by Prep-HPLC (column: Phenomenex C18 150×25 mm×10 μm; mobile phase: [water(NH4HCO3)-ACN]; B %: 31%-61%, 8 min) to afford Compound 2 (1.95 mg, 24% yield) as a white solid.

LCMS: Rt=0.556 min; (ESI positive ion) m/z: 494.1 (M+H)+(calculated: 494.16).

1H NMR (400 MHz, DMSO-d6) δ=8.43 (s, 1H), 8.26 (d, J=8.6 Hz, 2H), 7.74 (d, J=8.6 Hz, 2H), 7.52 (br d, J=6.6 Hz, 2H), 7.46-7.39 (m, 3H), 5.80 (d, J=4.8 Hz, 1H), 5.75 (s, 2H), 5.61 (s, 2H), 5.37 (d, J=5.8 Hz, 1H), 5.09 (d, J=5.6 Hz, 1H), 4.90-4.86 (m, 1H), 3.96-3.87 (m, 2H), 1.18 (d, J=6.4 Hz, 3H).

### Compound 3:

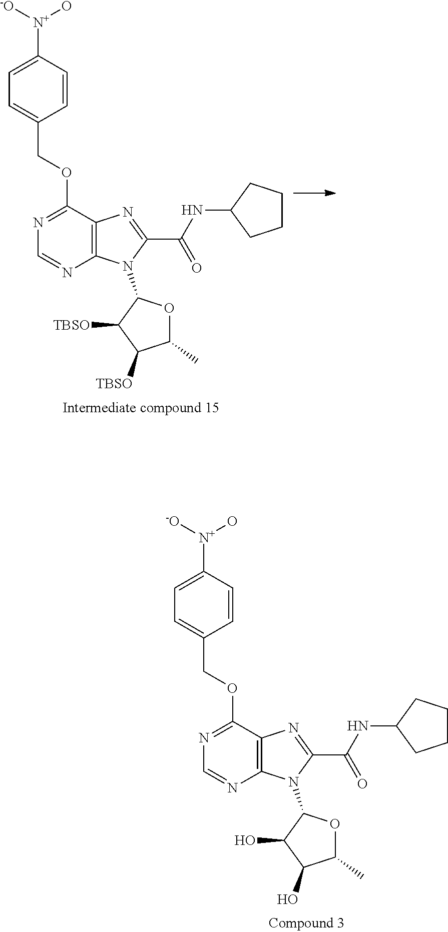

To a solution of Intermediate Compound 15 (65 mg, 89.41 μmol, 1 eq) in MeOH (1.5 mL) was added NH4F (66.23 mg, 1.79 mmol, 20 eq). The mixture was stirred at 65° C. for 12 hr and filtered. The filtrate was purified by Prep-HPLC (column: Waters xbridge 150×25 mm 10 m; mobile phase: [water (NH4HCO3)-ACN]; B %: 33%-63%, 8 min). The purified solution was lyophilized to afford Compound 3 (16.86 mg, 33.55 μmol, 37.53% yield, 99.2% purity) as a white solid.

LCMS: Rt=0.438 min; (ESI positive ion) m/z: 499.2 (M+H)+(calculated: 499.18).

1H NMR (400 MHz, DMSO-d6) δ=9.08 (d, J=7.6 Hz, 1H), 8.65 (s, 1H), 8.30-8.24 (m, 2H), 7.80-7.74 (m, 2H), 6.69 (d, J=4.2 Hz, 1H), 5.81 (s, 2H), 5.30 (d, J=5.6 Hz, 1H), 5.08 (d, J=6.0 Hz, 1H), 5.02-4.97 (m, 1H), 4.26-4.19 (m, 2H), 3.89 (quin, J=6.2 Hz, 1H), 1.93-1.83 (m, 2H), 1.73-1.65 (m, 2H), 1.64-1.51 (m, 4H), 1.28 (d, J=6.4 Hz, 3H).

### Compound 12:

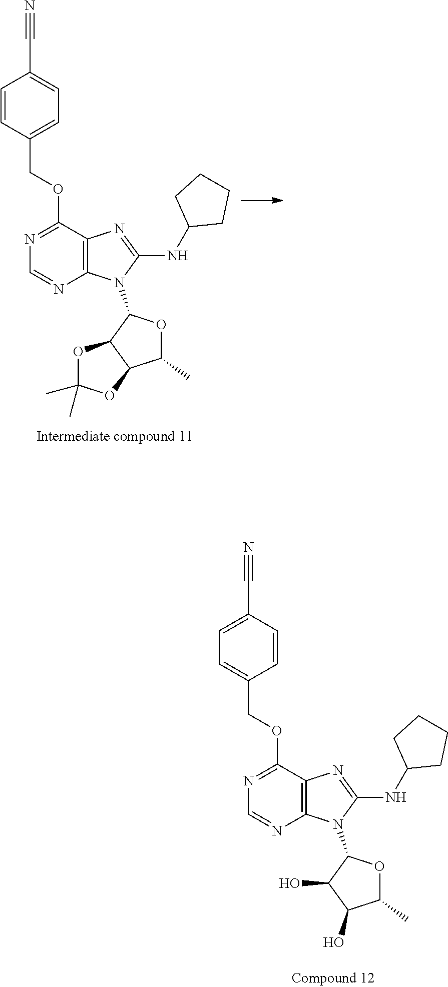 [SMILES: C[C@H]1O[C@@H](n2c(NC3CCCC3)nc3c(OCc4ccc(C#N)cc4)ncnc32)[C@@H]2OC(C)(C)O[C@@H]21 ; C[C@H]1O[C@@H](n2c(NC3CCCC3)nc3c(OCc4ccc(C#N)cc4)ncnc32)[C@H](O)[C@@H]1O]

A solution of Intermediate Compound 11 (48 mg, 97.85 μmol, 1 eq) in HCOOH/H2O=4/1(V/V) (2 mL) was stirred at 0° C. for 7 hr. The pH of the reaction mixture was adjusted with NH3·H2O to 7-8 at 0° C. Then the mixture was added H2O (10 mL) and extracted with EtOAc (20 mL×3). The organic layer phase was washed with brine, dried by Na2SO4, and concentrated to give a residue. The residue was purified by Prep-HPLC (column: Waters xbridge 150×25 mm×10 μm; mobile phase: [water (NH4HCO3)-ACN]; B %: 28%-58%, 8 min). The purified solution was lyophilized to afford Compound 12 (14.02 mg, 32% yield) as a white solid. LCMS: Rt=0.471 min; (ESI positive ion) m/z: 451.2 (M+H)+(calculated: 451.20).

1H NMR (400 MHz, DMSO-d6) δ=8.18 (s, 1H), 7.86 (d, J=8.4 Hz, 2H), 7.64 (d, J=8.2 Hz, 2H), 7.08 (d, J=7.0 Hz, 1H), 5.78 (d, J=4.2 Hz, 1H), 5.63 (s, 2H), 5.26 (d, J=5.2 Hz, 1H), 5.12-5.00 (m, 2H), 4.26-4.16 (m, 1H), 4.13 (q, J=5.6 Hz, 1H), 3.88 (quin, J=6.2 Hz, 1H), 2.02-1.89 (m, 2H), 1.75-1.64 (m, 2H), 1.61-1.49 (m, 4H), 1.25 (d, J=6.4 Hz, 3H).

### Compound 13:

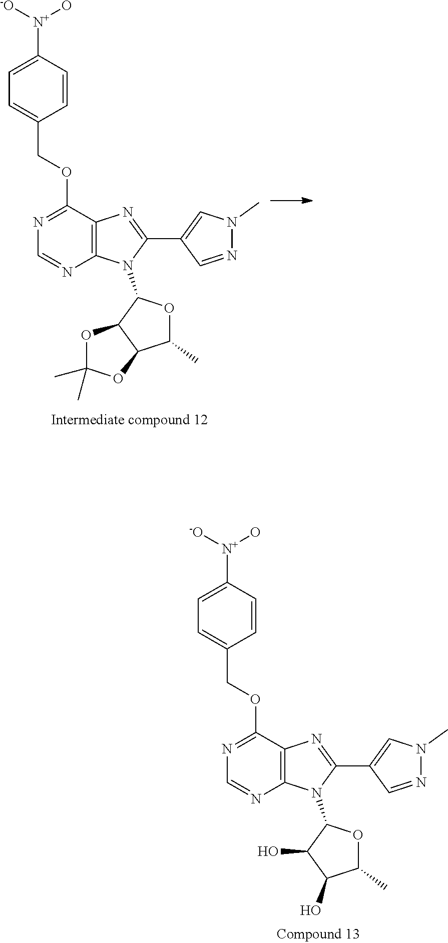

A solution of Intermediate Compound 12 (40 mg, 78.82 μmol, 1 eq) in HCOOH (0.8 mL) and H2O (0.2 mL) was stirred at 0° C. for 3 hr. The reaction mixture was concentrated under reduced pressure to give a residue. The residue was purified by Prep-HPLC (column: Phenomenex C18 150×25 mm×10 μm; mobile phase: [water (NH4HCO3)-ACN]; B %: 18%-48%, 8 min) and then lyophilized to afford Compound 13 (9.77 mg, 26% yield) as a white solid. LCMS: Rt=0.459 min; (ESI positive ion) m/z: 468.2 (M+H)+(calculated: 468.15).

1H NMR (400 MHz, DMSO-d6) δ=8.53 (s, 1H), 8.33 (s, 1H), 8.27 (br d, J=8.8 Hz, 2H), 7.95 (s, 1H), 7.76 (br d, J=8.6 Hz, 2H), 5.84 (d, J=4.6 Hz, 1H), 5.80 (s, 2H), 5.40-5.32 (m, 2H), 5.19 (d, J=5.4 Hz, 1H), 4.17-4.11 (m, 1H), 4.06-4.00 (m, 1H), 3.97 (s, 3H), 1.32 (br d, J=6.2 Hz, 3H).

### Compound 24:

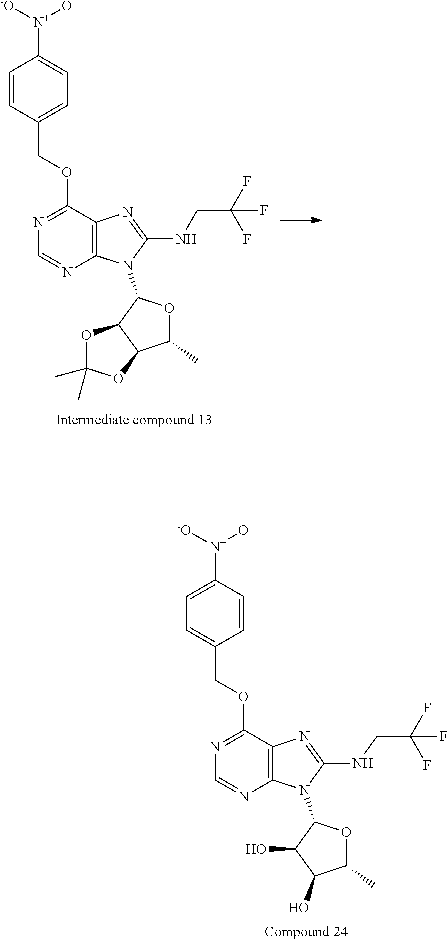

A solution of Intermediate Compound 13 (20 mg, 38.14 μmol, 1 eq) in H2O (0.2 mL) and HCOOH (0.8 mL) was stirred at 0° C. for 6 hr. NH3·H2O at 0° C. was added to the reaction mixture until the pH was 7. The mixture was then filtered and concentrated under reduced pressure to give a residue. The residue was purified by Prep-HPLC (column: Phenomenex C18 150×25 mm×10 μm; mobile phase: [water (NH4HCO3)-ACN]; B %: 30%-60%, 8 min) to afford Compound 24 (3 mg, 15% yield) as a white solid.

LCMS: Rt=0.455 min; (ESI positive ion) m/z: 485.1 (M+H)+(calculated: 485.13).

1H NMR (400 MHz, DMSO-d6) δ=8.29-8.25 (m, 2H), 8.24 (s, 1H), 7.96 (br t, J=6.4 Hz, 1H), 7.74-7.70 (m, 2H), 5.79-5.76 (m, 1H), 5.72-5.69 (m, 2H), 5.36-5.33 (m, 1H), 5.18-5.13 (m, 1H), 5.12-5.10 (m, 1H), 4.28-4.19 (m, 2H), 4.14-4.10 (m, 1H), 3.94-3.88 (m, 1H), 1.28-1.25 (m, 3H).

### Compound 75:

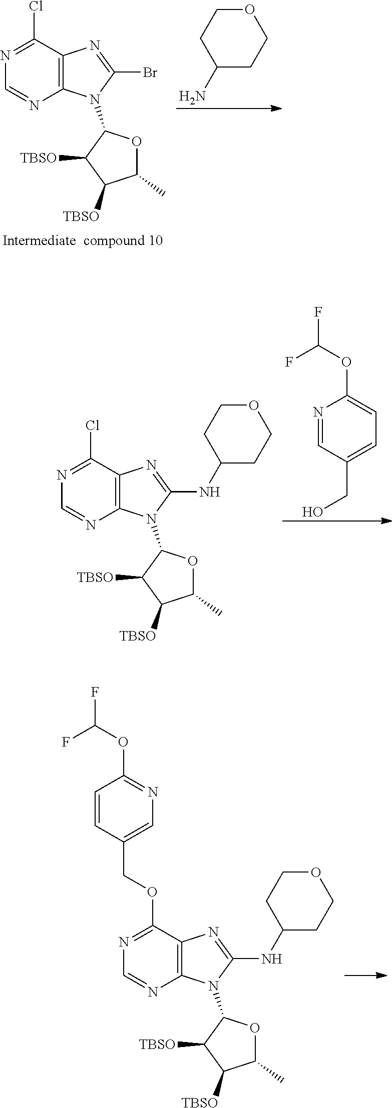 [SMILES: C[C@H]1O[C@@H](n2c(Br)nc3c(Cl)ncnc32)[C@H](O[Si](C)(C)C(C)(C)C)[C@@H]1O[Si](C)(C)C(C)(C)C ; NC1CCOCC1 ; C[C@H]1O[C@@H](n2c(NC3CCOCC3)nc3c(Cl)ncnc32)[C@H](O[Si](C)(C)C(C)(C)C)[C@@H]1O[Si](C)(C)C(C)(C)C ; OCc1ccc(OC(F)F)nc1 ; C[C@H]1O[C@@H](n2c(NC3CCOCC3)nc3c(OCc4ccc(OC(F)F)nc4)ncnc32)[C@H](O[Si](C)(C)C(C)(C)C)[C@@H]1O[Si](C)(C)C(C)(C)C]

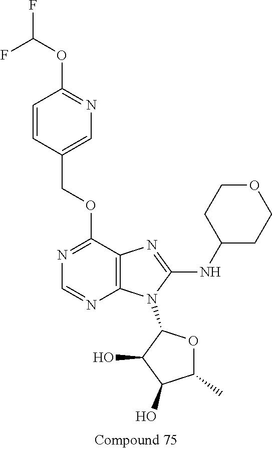 [SMILES: C[C@H]1O[C@@H](n2c(NC3CCOCC3)nc3c(OCc4ccc(OC(F)F)nc4)ncnc32)[C@H](O)[C@@H]1O]

To a solution of Intermediate Compound 10 (64 g, 110.71 mmol, 1 eq) in EtOH (640 mL) was added TEA (33.61 g, 332.13 mmol, 46.23 mL, 3 eq) and tetrahydro-2H-pyran-4-amine (11.20 g, 110.71 mmol, 1 eq). The mixture was stirred at 80° C. for 1 hr. The reaction mixture was filtered and concentrated under vacuum. The crude product was purified by reversed-phase HPLC (neutral condition) to afford the corresponding amine (27.6 g, 42% yield) as a yellow solid.

LCMS: Rt=1.067 min; (ESI positive ion) m/z: 598.2 (M+H)+(calculated: 598.29).

1H NMR (400 MHz, DMSO-d6) δ=8.40 (s, 1H), 5.84 (d, J=4.4 Hz, 1H), 5.62 (br d, J=7.6 Hz, 1H), 5.07 (t, J=4.4 Hz, 1H), 4.30-4.20 (m, 2H), 4.05-3.92 (m, 3H), 3.65-3.54 (m, 2H), 2.23-2.09 (m, 2H), 2.00 (s, 1H), 1.63-1.50 (m, 2H), 1.40 (d, J=6.6 Hz, 3H), 0.94 (s, 9H), 0.78 (s, 9H), 0.11 (d, J=11.8 Hz, 6H), 0.00 (s, 3H), −0.21 (s, 3H).

A mixture of the above amine (27.6 g, 46.13 mmol, 1 eq), (6-(difluoromethoxy)pyridin-3-yl)methanol (8.48 g, 48.44 mmol, 1.05 eq), Pd2(dba)3 (4.22 g, 4.61 mmol, 0.1 eq), Xantphos (5.34 g, 9.23 mmol, 0.2 eq) and Cs2CO3 (37.57 g, 115.32 mmol, 2.5 eq) in toluene (300 mL) was degassed and purged with N2 3 times. The mixture was then stirred at 80° C. for 12 hr under N2 atmosphere. The reaction mixture was filtered and concentrated under vacuum. The resultant residue was purified by flash silica gel chromatography (ISCO®; 120 g SepaFlash® Silica Flash Column, Eluent of 0-30% Ethyl acetate/Petroleum ether gradient @100 mL/min) to afford the corresponding ether (31 g, 91% yield) as a yellow solid.

LCMS: Rt=0.712 min; (ESI positive ion) m/z: 737.5 (M+H)+(calculated: 737.36).

To a solution of the above ether (30.6 g, 41.52 mmol, 1 eq) in MeOH (280 mL) was added NH4F (30.76 g, 830.40 mmol, 20 eq). The mixture was stirred at 80° C. for 12 hr. The reaction mixture was then filtered and concentrated under vacuum. The residue was purified by flash silica gel chromatography (ISCO®; 330 g SepaFlash® Silica Flash Column, Eluent of 0-1% MeOH/Ethyl acetate @100 mL/min) to afford Compound 75 (12.1 g, 58% yield) as a white solid. LCMS: Rt=0.474 min; (ESI positive ion) m/z: 509.2 (M+H)+(calculated: 509.19).

1H NMR (400 MHz, DMSO-d6) δ=8.42 (d, J=2.2 Hz, 1H), 8.23 (s, 1H), 8.06 (dd, J=2.4, 8.4 Hz, 1H), 7.91-7.52 (m, 1H), 7.18-7.10 (m, 2H), 5.76 (d, J=4.2 Hz, 1H), 5.54 (s, 2H), 5.30 (d, J=5.2 Hz, 1H), 5.14-5.06 (m, 2H), 4.18-4.12 (m, 1H), 4.02-3.83 (m, 4H), 3.42-3.36 (m, 2H), 2.08 (s, 1H), 1.87 (ddd, J=2.0, 4.4, 6.4 Hz, 2H), 1.64-1.51 (m, 2H), 1.25 (d, J=6.4 Hz, 3H).

### Compound 135:

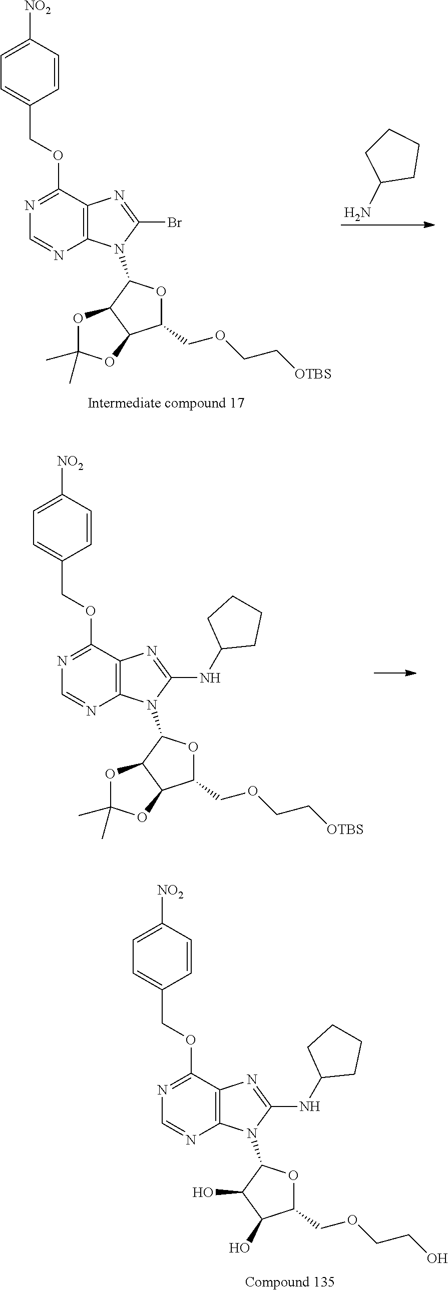 [SMILES: CC1(C)O[C@@H]2[C@H](O1)[C@@H](COCCO[Si](C)(C)C(C)(C)C)O[C@H]2n1c(Br)nc2c(OCc3ccc([N+](=O)[O-])cc3)ncnc21 ; NC1CCCC1 ; CC1(C)O[C@@H]2[C@H](O1)[C@@H](COCCO[Si](C)(C)C(C)(C)C)O[C@H]2n1c(NC2CCCC2)nc2c(OCc3ccc([N+](=O)[O-])cc3)ncnc21 ; O=[N+]([O-])c1ccc(COc2ncnc3c2nc(NC2CCCC2)n3[C@@H]2O[C@H](COCCO)[C@@H](O)[C@H]2O)cc1]

A mixture of Intermediate Compound 17 (117 mg, 171.90 μmol, 1 eq), cyclopentanamine (43.91 mg, 515.71 μmol, 50.88 μL, 3 eq and TEA (86.97 mg, 859.51 μmol, 119.63 μL, 5 eq in DMSO (5 mL) was stirred at 110° C. for 16 hr. The mixture was poured into water (20 mL) and the aqueous phase was extracted with ethyl acetate (10 mL×2). The combined organic phase was washed with brine (20 mL×2), dried with anhydrous Na2SO4, filtered and concentrated under vacuum. The resultant residue was purified by Prep-TLC (Petroleum ether/Ethyl acetate=1/1, Rf=0.5) to afford the corresponding amine (78 mg, 112.75 μmol, 65.59% yield, 99% purity) as a yellow oil.

LCMS: Rt=0.644 min; (ESI positive ion) m/z: 685.4(M+H)+(calculated: 685.33).

A solution of the preceding amine compound (78 mg, 113.89 μmol, 1 eq) in HCOOH (1 mL) and water (0.25 mL) was stirred at 20° C. for 2 hr. The mixture was then concentrated under vacuum. The resultant residue was treated with K2CO3 (10 mg)/MeOH (1 mL) and the mixture was stirred at 20° C. for 10 min. The mixture was filtered. The filtrate was purified by Prep-TLC (DCM/MeOH=10/1) followed by Prep-HPLC (column: Phenomenex C18 150×25 mm×10 μm; mobile phase: [water (NH4HCO3)-ACN]; B %: 25%-55%, 8 min) to afford Compound 135 (30.38 mg, 50% yield) as a white solid.

LCMS: Rt=0.464 min; (ESI positive ion) m/z: 531.1(M+H)+(calculated: 531.21). 1H NMR (400 MHz, DMSO-d6) δ=8.25 (d, J=8.8 Hz, 2H), 8.18 (s, 1H), 7.71 (d, J=8.8 Hz, 2H), 6.85 (br d, J=7.0 Hz, 1H), 5.87 (d, J=5.6 Hz, 1H), 5.70 (s, 2H), 5.31 (br s, 1H), 5.18 (br s, 1H), 4.91 (br s, 1H), 4.72-4.44 (m, 1H), 4.27-4.19 (m, 2H), 3.99-3.94 (m, 1H), 3.71 (dd, J=3.6, 10.8 Hz, 1H), 3.55 (dd, J=5.8, 10.8 Hz, 1H), 3.48-3.42 (m, 4H), 1.98 (br d, J=3.4 Hz, 2H), 1.69 (br s, 2H), 1.60-1.51 (m, 4H).

### Compound 151:

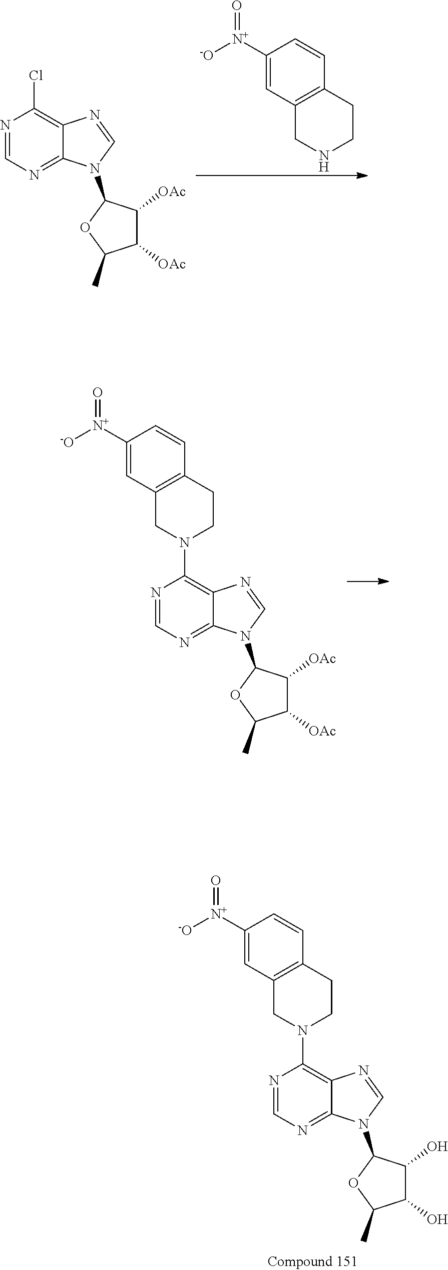

To a mixture of Intermediate Compound 1 (100 mg, 281.89 μmol, 1 eq) and 7-nitro-1,2,3,4-tetrahydroisoquinoline (75.34 mg, 422.84 μmol, 1.5 eq) in ethanol (2 mL) was added TEA (228.20 mg, 2.26 mmol, 313.89 μL, 8 eq). The mixture was stirred at 80° C. for 2 hr. The solvent was removed under reduced pressure to afford 166 mg of the crude compound as a yellow solid.

LCMS: Rt=0.590 min; (ESI positive ion) m/z: 497.3(M+H)+(calculated: 497.17). A mixture of the preceding crude compound (166 mg, 334.36 μmol, 1 eq) and NH3·H2O (1.82 g, 14.54 mmol, 2 mL, 28% purity, 43.48 eq) in MeOH (2 mL) was stirred at 20° C. for 0.5 hr. The solvent was removed under reduced pressure to give a residue. The residue was purified by Prep-HPLC (column: Phenomenex luna C18 150×25 mm×10 μm; mobile phase: [water(FA)-ACN]; B %: 22%-52%, 9 min) to afford Compound 151 (23.5 mg, 17% yield) as a white solid.

LCMS: Rt=0.439 min; (ESI positive ion) m/z: 413.1 (M+H)+(calculated: 413.15).

1H NMR (400 MHz, DMSO-d6) δ=8.44 (s, 1H), 8.33 (s, 1H), 8.19 (br s, 1H), 8.04 (dd, J=2.2, 8.4 Hz, 1H), 7.47 (d, J=8.6 Hz, 1H), 5.90 (d, J=4.8 Hz, 1H), 5.56-5.23 (m, 3H), 5.18 (br d, J=4.4 Hz, 1H), 4.72-4.40 (m, 3H), 3.97 (br d, J=3.8 Hz, 2H), 3.07 (br t, J=5.6 Hz, 2H), 1.30 (br d, J=5.8 Hz, 3H)

### Compound 152:

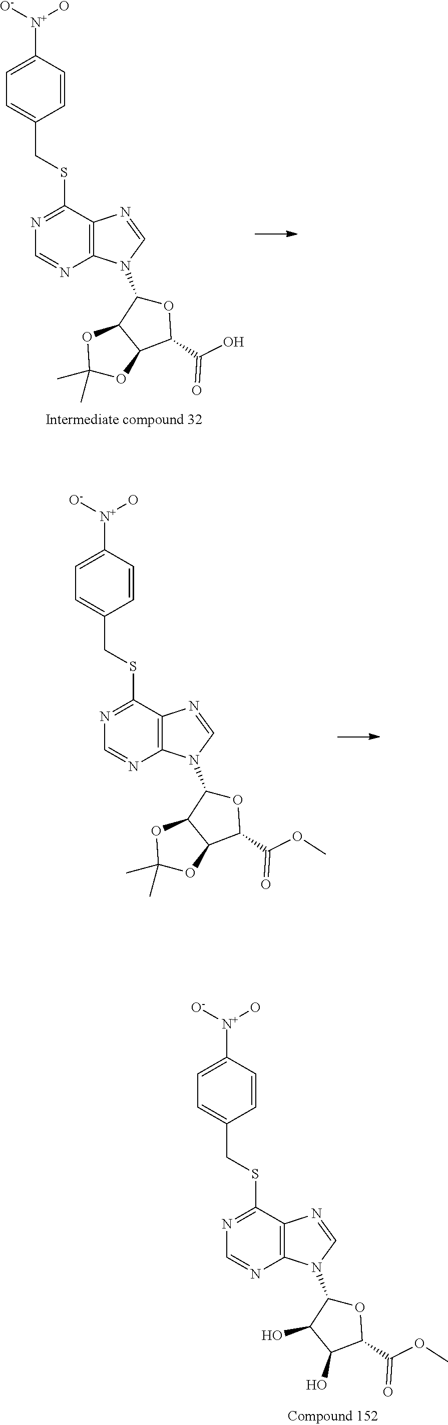 [SMILES: CC1(C)O[C@@H]2[C@H](O1)[C@@H](C(=O)O)O[C@H]2n1cnc2c(SCc3ccc([NH+]([O-])O)cc3)ncnc21 ; COC(=O)[C@H]1O[C@@H](n2cnc3c(SCc4ccc([NH+]([O-])O)cc4)ncnc32)[C@@H]2OC(C)(C)O[C@@H]21 ; COC(=O)[C@H]1O[C@@H](n2cnc3c(SCc4ccc([NH+]([O-])O)cc4)ncnc32)[C@H](O)[C@@H]1O]

To a solution of Intermediate Compound 32 (574 mg, 1.21 mmol, 1 eq) in MeCN (6 mL) was added iodomethane (516.24 mg, 3.64 mmol, 226.42 μL, 3 eq) and K2CO3 (502.66 mg, 3.64 mmol, 3 eq). The mixture was stirred at 60° C. for 2 hr. To the reaction mixture was added EtOAc (30 mL). The mixture was then stirred at 20° C. for 15 min and filtered. The filtrate was concentrated to give a residue. The residue was purified by column chromatography (SiO2, Petroleum ether/Ethyl acetate=5/1 to 1/1) to afford the corresponding ester compound (598 mg, crude) as a yellow solid.

LCMS: Rt=0.956 min; (ESI positive ion) m/z: 488.3 (M+H)+(calculated: 488.12).

1H NMR (400 MHz, DMSO-d6) δ=8.70 (s, 1H), 8.62 (s, 1H), 8.16 (d, J=8.7 Hz, 2H), 7.74 (d, J=8.7 Hz, 2H), 6.49 (s, 1H), 5.60 (dd, J=1.3, 5.9 Hz, 1H), 5.51 (d, J=5.9 Hz, 1H), 4.91 (s, 1H), 4.84-4.72 (m, 2H), 3.27 (s, 3H), 1.52 (s, 3H), 1.35 (s, 3H).

A mixture of the preceding ester compound (60 mg, 123.08 μmol, 1 eq) in water (0.2 mL) was added TFA (1.23 g, 10.81 mmol, 0.8 mL, 87.79 eq) at 0° C. The mixture was stirred at 20° C. for 3 hr. The solvent was removed under reduced pressure to give a residue. The residue was purified by Prep-HPLC (column: Phenomenex Gemini-NX C18 75×30 mm×3 μm; mobile phase: [water(0.225% FA)-ACN]; B %: 25%-55%, 7 min). The purified solution was lyophilized to afford Compound 152 (21.79 mg, 38% yield) as a white solid.

LCMS: Rt=0.848 min; (ESI positive ion) m/z: 448.2 (M+H)+(calculated: 448.08).

1H NMR (400 MHz, DMSO-d6) δ=8.80 (s, 1H), 8.75 (s, 1H), 8.17 (d, J=8.9 Hz, 2H), 7.75 (d, J=8.8 Hz, 2H), 6.15 (d, J=6.1 Hz, 1H), 5.92 (br d, J=4.5 Hz, 1H), 5.78 (br d, J=5.4 Hz, 1H), 4.81 (s, 2H), 4.68 (q, J=5.2 Hz, 1H), 4.53 (d, J=2.8 Hz, 1H), 4.41 (br d, J=2.9 Hz, 1H), 3.72 (s, 3H).

### Compound 156:

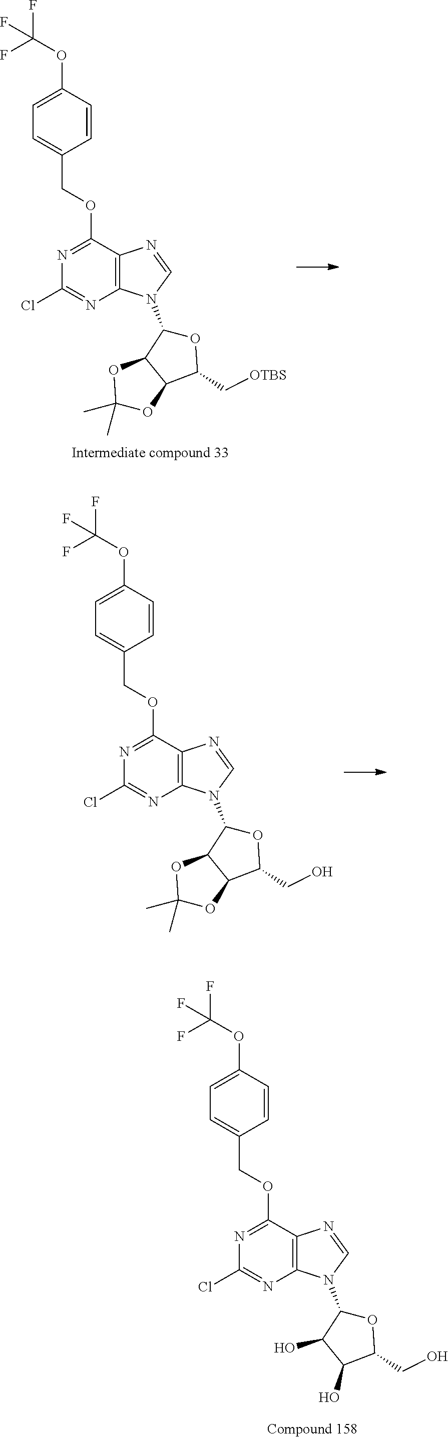 [SMILES: CC1(C)O[C@@H]2[C@H](O1)[C@@H](CO[Si](C)(C)C(C)(C)C)O[C@H]2n1cnc2c(OCc3ccc(OC(F)(F)F)cc3)nc(Cl)nc21 ; CC1(C)O[C@@H]2[C@H](O1)[C@@H](CO)O[C@H]2n1cnc2c(OCc3ccc(OC(F)(F)F)cc3)nc(Cl)nc21 ; OC[C@H]1O[C@@H](n2cnc3c(OCc4ccc(OC(F)(F)F)cc4)nc(Cl)nc32)[C@H](O)[C@@H]1O]

To a solution of Intermediate Compound 33 (1, 150 mg, 237.67 μmol, 1 eq) in MeOH (2 mL) was added NH4F (88.03 mg, 2.38 mmol, 10 eq). The mixture was stirred at 60° C. for 1 hour. The reaction mixture was concentrated under reduced pressure to give a residue. The residue was purified by Prep-TLC (SiO2, Petroleum ether:Ethyl acetate=5:1) to afford the corresponding alcohol (120 mg, 960% yield) as a yellow oil.

LCMS: Rt=0.997 min; (ESI positive ion) m/z: 517.1 (M+H)+(calculated: 517.10).

To a solution of the above alcohol compound (50 mg, 96.74 μmol, 1 eq) in water (0.1 mL) was added formic acid (488.00 mg, 10.60 mmol, 0.4 mL, 109.60 eq) at 0° C. The mixture was stirred at 20° C. for 0.5 hour. NH3·H2O (2 mL) was added to the reaction mixture at 0° C. until the pH reached 7. The mixture was then filtered and concentrated under reduced pressure to give a residue. The residue was purified by Prep-HPLC (column: Phenomenex C18 75×30 mm×3 μm; mobile phase: [water(FA)-ACN]; B %: 35%-65%, 7 min) to afford Compound 156 (24.04 mg, 50% yield) as a white solid.

LCMS: Rt=0.910 min; (ESI positive ion) m/z: 476.9 (M+H)+(calculated: 477.07).

1H NMR (400 MHz, MeOD-d4) δ=8.57 (s, 1H), 7.67 (d, J=8.6 Hz, 2H), 7.31 (d, J=8.3 Hz, 2H), 6.04 (d, J=5.4 Hz, 1H), 5.68 (s, 2H), 4.65 (t, J=5.2 Hz, 1H), 4.34 (t, J=4.4 Hz, 1H), 4.15 (q, J=3.2 Hz, 1H), 3.93-3.85 (m, 1H), 3.81-3.74 (m, 1H)

### Compound 157:

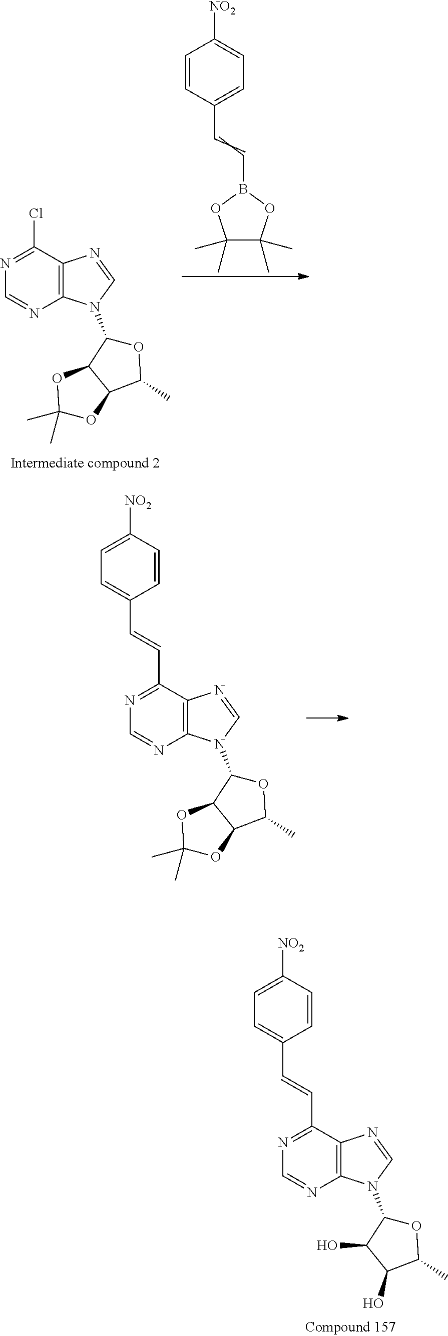 [SMILES: C[C@H]1O[C@@H](n2cnc3c(Cl)ncnc32)[C@@H]2OC(C)(C)O[C@@H]21 ; CC1(C)OB(C=Cc2ccc([N+](=O)[O-])cc2)OC1(C)C ; C[C@H]1O[C@@H](n2cnc3c(/C=C/c4ccc([N+](=O)[O-])cc4)ncnc32)[C@@H]2OC(C)(C)O[C@@H]21 ; C[C@H]1O[C@@H](n2cnc3c(/C=C/c4ccc([N+](=O)[O-])cc4)ncnc32)[C@H](O)[C@@H]1O]

A mixture of Intermediate Compound 2 (100 mg, 321.82 μmol, 1 eq), 4,4,5,5-tetramethyl-2-(4-nitrostyryl)-1,3,2-dioxaborolane (4, 132.80 mg, 482.73 μmol, 1.5 eq), Pd(dppf)Cl2·CH2Cl2 (26.28 mg, 32.18 μmol, 0.1 eq), Na2CO3 (85.27 mg, 804.55 μmol, 2.5 eq) in dioxane (1 mL), and H2O (0.1 mL) was degassed and purged with N2 3 times. The mixture was stirred at 80° C. for 16 hours under N2 atmosphere. The mixture was then poured into 80 mL of water, extracted with EtOAc (80 mL×3) and the combined organic layer was condensed. The resultant residue was purified by Prep-TLC (SiO2, Petroleum ether:Ethyl acetate=2:1) to afford the corresponding coupled compound (100 mg, 73% yield) as a yellow solid.

LCMS: Rt=0.951 min; (ESI positive ion) m/z: 424.1 (M+H)+(calculated: 424.15).

1H NMR (400 MHz, CDCl₃) δ=9.00 (s, 1H), 8.44 (d, J=16.3 Hz, 1H), 8.33-8.22 (m, 3H), 7.90-7.81 (m, 3H), 6.14 (d, J=2.4 Hz, 1H), 5.58 (dd, J=2.3, 6.5 Hz, 1H), 4.83 (dd, J=3.4, 6.4 Hz, 1H), 4.45 (dq, J=3.7, 6.6 Hz, 1H), 1.65 (s, 3H), 1.43-1.39 (m, 6H)

A solution of the above coupled compound (80.00 mg, 188.94 μmol, 1 eq) in formic acid (3.90 g, 84.82 mmol, 3.20 mL, 448.95 eq) and water (800.00 mg, 44.41 mmol, 800.00 μL, 235.03 eq) was stirred at 20° C. for 1 hour. The reaction mixture was filtered and concentrated under reduced pressure to give a residue. The residue was purified by Prep-HPLC (column: Unisil 3-100 C18 Ultra 150×50 mm×3 μm; mobile phase: [water(FA)-ACN]; B %: 22%-52%, 15 min) to afford Compound 157 (37.05 mg, 51% yield) as a white solid.

LCMS: Rt=0.836 min; (ESI positive ion) m/z: 384.2 (M+H)+(calculated: 384.12).

1H NMR (400 MHz, DMSO-d6) δ=8.97 (s, 1H), 8.87 (s, 1H), 8.47 (d, J=16.3 Hz, 1H), 8.28 (d, J=8.7 Hz, 2H), 8.10 (d, J=8.4 Hz, 2H), 7.90 (d, J=16.2 Hz, 1H), 6.02 (d, J=5.0 Hz, 1H), 5.56 (br s, 1H), 5.27 (br s, 1H), 4.76 (br d, J=3.9 Hz, 1H), 4.09-4.00 (m, 2H), 1.35 (d, J=6.3 Hz, 3H).

### Compound 158:

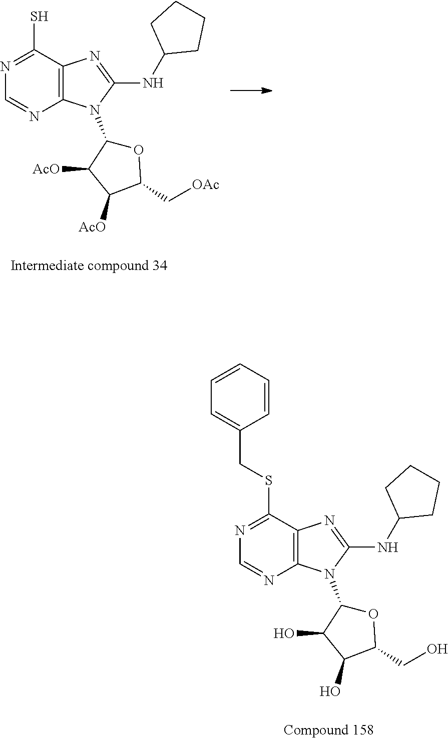 [SMILES: CC(=O)OC[C@H]1O[C@@H](n2c(NC3CCCC3)nc3c(S)ncnc32)[C@H](OC(C)=O)[C@@H]1OC(C)=O ; OC[C@H]1O[C@@H](n2c(NC3CCCC3)nc3c(SCc4ccccc4)ncnc32)[C@H](O)[C@@H]1O]

To a solution of Intermediate Compound 34 (148 mg, 299.88 μmol, 1 eq) in DMF (1.5 mL) was added K2CO3 (41.45 mg, 299.88 μmol, 1 eq) and benzyl bromine (61.55 mg, 359.85 mol, 42.74 μL, 1.2 eq). The mixture was stirred at 20° C. for 12 hr. K2CO3 (124.34 mg, 899.64 mol, 3 eq) and MeOH (0.5 mL) were added to the reaction mixture. The mixture was stirred at 20° C. for 16 hr. The filtrate was purified by Prep-HPLC (column: Phenomenex luna C18 150×25 mm×10 μm; mobile phase:[water(FA)-ACN]; B %: 36%-66%, 10 min). The purified solution was lyophilized to afford Compound 158 (13.98 mg, 10% yield) as a light yellow solid.

LCMS: Rt=0.511 min; (ESI positive ion) m/z: 458.1 (M+H)+(calculated: 458.18).

1H NMR (400 MHz, DMSO-d6) δ=8.41 (s, 1H), 7.42 (d, J=7.4 Hz, 2H), 7.33-7.27 (m, 3H), 7.26-7.20 (m, 1H), 6.01 (d, J=7.4 Hz, 1H), 5.73 (t, J=4.8 Hz, 1H), 5.32 (d, J=6.4 Hz, 1H), 5.18 (d, J=4.2 Hz, 1H), 4.65-4.50 (m, 3H), 4.32-4.21 (m, 1H), 4.15-4.08 (m, 1H), 3.98 (d, J=1.8 Hz, 1H), 3.64 (br d, J=3.0 Hz, 2H), 2.00-1.90 (m, 2H), 1.72-1.64 (m, 2H), 1.59-1.50 (m, 4H).

### Compound 159:

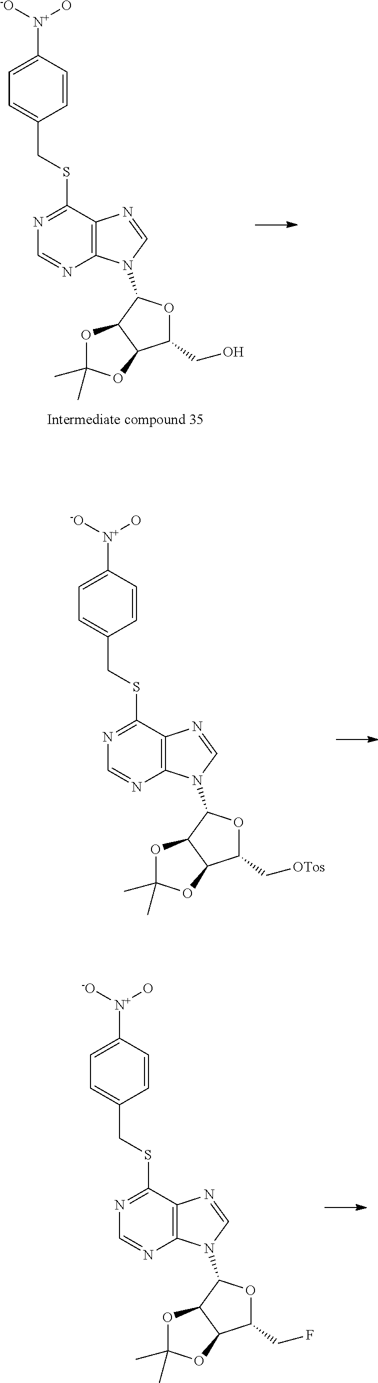

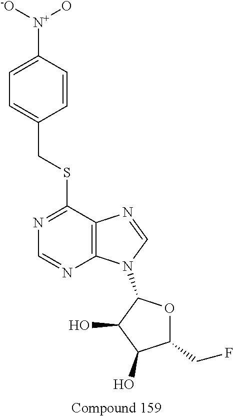

To a solution of Intermediate Compound 35 (200 mg, 435.28 μmol, 1 eq) in dry pyridine (5 mL) was added p-toluenesulfonyl chloride (248.95 mg, 1.31 mmol, 3 eq). The mixture was stirred at 40° C. for 4 hr and then concentrated to give a residue. The residue was dissolved in an aqueous solution of 1N HCl (80 mL) and extracted with EtOAc (60 mL×3). The combined organic layer was washed with brine, dried by Na2SO4, and concentrated to afford the corresponding tosylated compound (210 mg, crude) as a yellow oil.

LCMS: Rt=1.014 min; (ESI positive ion) m/z: 614.20 (M+H)+(calculated: 614.14).

To a solution of the above tosylated compound (157 mg, 255.84 μmol, 1 eq) in tert-amyl alcohol (5 mL) was added cesium fluoride (116.59 mg, 767.52 μmol, 28.30 μL, 3 eq). The mixture was stirred at 120° C. for 2 hr and then poured into ice-H2O (10 mL). The mixture was extracted with EtOAc (5 mL×2) and the combined organic phase was washed with brine (10 mL), dried over anhydrous Na2SO4, filtered and concentrated under vacuum. The resultant residue was purified by Prep-HPLC (column: Unisil 3-100 C18 Ultra 150×50 mm×3 μm; mobile phase: [water (0.225% FA)-ACN]; B %: 45%-75%, 10 min) to afford the corresponding fluorinated compound (15 mg, 13% yield, 100% purity) as a yellow solid.

LCMS: Rt=0.950 min; (ESI positive ion) m/z: 461.90 (M+H)+(calculated: 462.12).

A solution of the above fluorinated compound (11 mg, 23.84 μmol, 1 eq) in TFA (0.4 mL) and water (0.1 mL) was stirred at 0° C. for 5 min. The mixture was then warmed and stirred at 20° C. for 55 min. The mixture was concentrated under vacuum, and the resultant residue was purified by Prep-HPLC (column: Unisil 3-100 C18 Ultra 150×50 mm×3 μm; mobile phase: [water(0.225% FA)-ACN]; B %: 27%-57%, 10 min) to afford Compound 159 (6.36 mg, 62% yield) as a white solid.

LCMS: Rt=0.833 min; (ESI positive ion) m/z: 421.90 (M+H)+(calculated: 422.09).

1H NMR (400 MHz, DMSO-d6) δ=8.79 (s, 1H), 8.62 (s, 1H), 8.17 (d, J=8.6 Hz, 2H), 7.75 (d, J=8.6 Hz, 2H), 6.03 (d, J=4.8 Hz, 1H), 5.84-5.66 (m, 1H), 5.63-5.44 (m, 1H), 4.80 (s, 2H), 4.74-4.69 (m, 1H), 4.65-4.58 (m, 2H), 4.28 (br t, J=4.8 Hz, 1H), 4.19-4.10 (m, 1H).

### Compound 161:

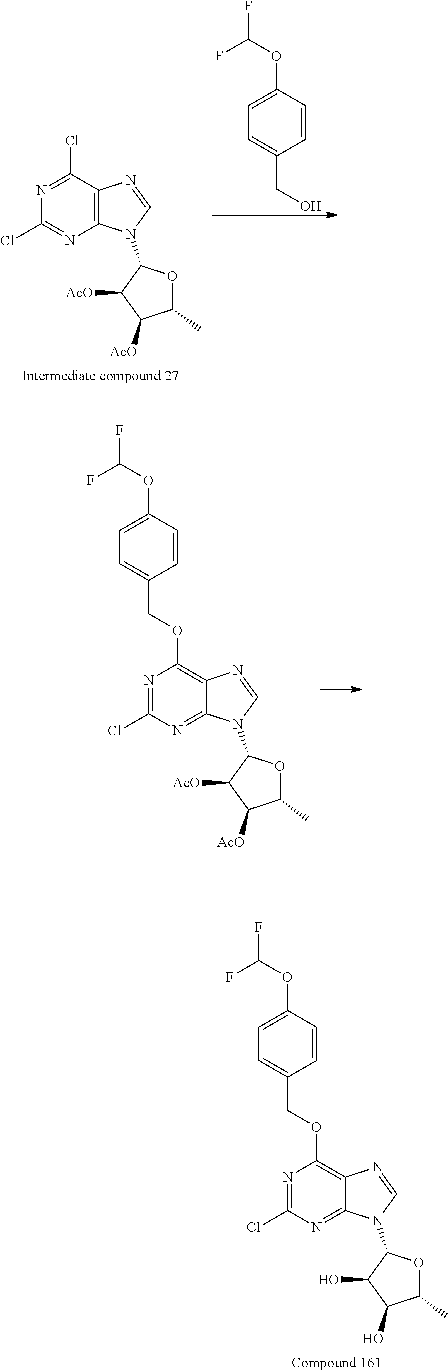 [SMILES: CC(=O)O[C@@H]1[C@H](OC(C)=O)[C@@H](C)O[C@H]1n1cnc2c(Cl)nc(Cl)nc21 ; OCc1ccc(OC(F)F)cc1 ; CC(=O)O[C@@H]1[C@H](OC(C)=O)[C@@H](C)O[C@H]1n1cnc2c(OCc3ccc(OC(F)F)cc3)nc(Cl)nc21 ; C[C@H]1O[C@@H](n2cnc3c(OCc4ccc(OC(F)F)cc4)nc(Cl)nc32)[C@H](O)[C@@H]1O]

To a solution of Intermediate Compound 27 (100 mg, 256.94 μmol, 1 eq) and (4-(difluoromethoxy)phenyl)methanol (44.75 mg, 256.94 μmol, 1 eq) in toluene (2 mL) was added Cs2CO3 (209.29 mg, 642.36 μmol, 2.5 eq), Xantphos (29.73 mg, 51.39 μmol, 0.2 eq) and Pd2(dba)3 (23.53 mg, 25.69 μmol, 0.1 eq). The mixture was stirred at 80° C. for 0.5 hr. The reaction mixture was then filtered using silica gel. The filtrate was removed under reduced pressure to afford the corresponding ether compound (40 mg, crude) as a green oil.

LCMS: Rt=0.942 min; (ESI positive ion) m/z: 527.10 (M+H)+(calculated: 527.11).

To a solution of the above ether compound (40 mg, 75.92 μmol, 1 eq) in MeOH (1 mL) was added NH3·H2O (910.00 mg, 7.27 mmol, 1.00 mL, 28% purity, 95.77 eq). The mixture was stirred at 20° C. for 1 hr and then concentrated to give a residue. The residue was purified by Prep-HPLC (column: Waters Xbridge 150×25 mm×5 μm; mobile phase: [water (NH4HCO3)-ACN]; B %: 36%-66%, 7 min). The purified solution was lyophilized to afford Compound 161 (21.15 mg, 62% yield) as a white solid.

LCMS: Rt=0.922 min; (ESI positive ion) m/z: 443.00 (M+H)+(calculated: 443.09).

1H NMR (400 MHz, DMSO-d6) δ=8.62 (s, 1H), 7.60 (d, J=8.6 Hz, 2H), 7.45-7.07 (m, 3H), 5.88 (d, J=5.1 Hz, 1H), 5.60 (s, 2H), 5.50 (d, J=5.8 Hz, 1H), 5.23 (d, J=5.4 Hz, 1H), 4.61 (q, J=5.2 Hz, 1H), 4.06-3.93 (m, 2H), 1.32 (d, J=6.4 Hz, 3H).

### Compound 162:

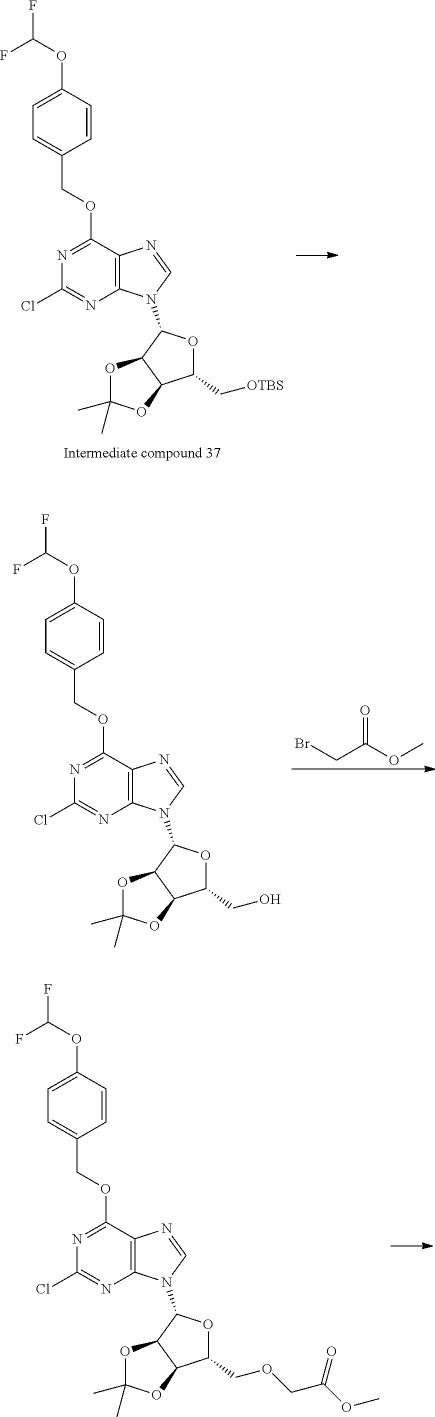

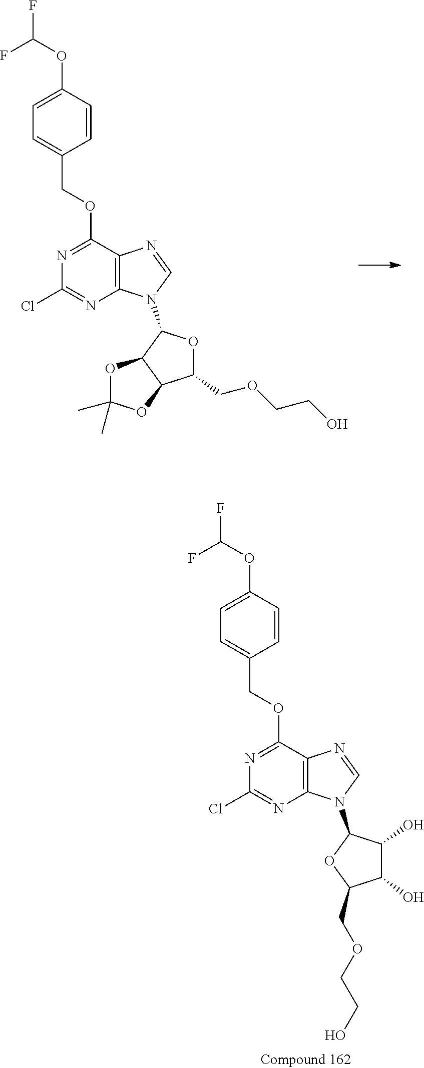 [SMILES: CC1(C)O[C@@H]2[C@H](O1)[C@@H](COCCO)O[C@H]2n1cnc2c(OCc3ccc(OC(F)F)cc3)nc(Cl)nc21 ; OCCOC[C@H]1O[C@@H](n2cnc3c(OCc4ccc(OC(F)F)cc4)nc(Cl)nc32)[C@H](O)[C@@H]1O]

To a solution of Intermediate Compound 37 (240 mg, 391.44 μmol, 1 eq) in MeOH (5 mL) was added NH4F (144.98 mg, 3.91 mmol, 10 eq). The mixture was stirred at 60° C. for 1 hour and then filtered and concentrated under reduced pressure to give a residue. The residue was purified by column chromatography (SiO2, Petroleum ether:Ethyl acetate=1:0 to 1:1) to afford the corresponding alcohol compound (107 mg, 55% yield) as a yellow oil.

LCMS: Rt=0.947 min; (ESI positive ion) m/z: 499.1 (M+H)+(calculated: 499.11).

To a solution of the above alcohol compound (100 mg, 200.46 μmol, 1 eq) and methyl 2-bromoacetate (5, 46.00 mg, 300.68 μmol, 28.39 μL, 1.5 eq) in DMF (2 mL) was added sodium hydride (12.03 mg, 300.68 μmol, 60% purity, 1.5 eq) at 0° C. The mixture was stirred at 0° C. for 5 hours and then poured into ice-water (10 mL). The aqueous phase was extracted with ethyl acetate (10 mL×3). The combined organic phase was washed with brine (5 mL), dried with anhydrous Na2SO4, filtered and concentrated under vacuum. The residue was purified by Prep-TLC (SiO2, DCM:MeOH=10:1) to afford the corresponding ether compound (67 mg, 57% yield) as a white solid.

LCMS: Rt=1.027 min; (ESI positive ion) m/z: 571.0 (M+H)+(calculated: 571.13).

To a solution of the above ether compound (67 mg, 117.35 μmol, 1 eq) in THE (1 mL) was added LiBH4 (2 M, 64.54 μL, 1.1 eq) at 0° C. The mixture was stirred at 0° C. for 1 hour. The mixture was then added to ice-water (10 mL) dropwise at 0° C. and stirred for 5 min. The mixture was extracted with EtOAc (5 mL×2) and the combined organic phase was washed with brine (10 mL), dried with anhydrous Na2SO4, filtered and concentrated under vacuum. The residue was purified by Prep-TLC (SiO2, Petroleum ether:Ethyl acetate=1:1) to afford the corresponding alcohol compound (7, 60 mg, 94% yield) as a white solid.

LCMS: Rt=0.974 min; (ESI positive ion) m/z: 545.2 (M+H)+(calculated: 545.14).

1H NMR (400 MHz, MeOD-d4) δ=8.54 (s, 1H), 7.64-7.45 (m, 2H), 7.20-7.11 (m, 2H), 7.04-6.62 (m, 1H), 6.23 (d, J=2.6 Hz, 1H), 5.64 (s, 2H), 5.35-5.32 (m, 1H), 5.10-5.04 (m, 1H), 4.53-4.48 (m, 1H), 3.75-3.49 (m, 6H), 1.61-1.37 (m, 6H)

To a solution of the above alcohol compound (50 mg, 92.10 μmol, 1 eq) in water (0.1 mL) was added formic acid (4.42 mg, 92.10 μmol, 0.4 mL, 1 eq) at 0° C. The mixture was stirred at 20° C. for 1 hour. NH3·H2O (0.1 ml) and MeOH (0.5 ml) were then added and the mixture stirred for 10 minutes at 20° C. The reaction mixture was filtered and concentrated under reduced pressure to give a residue. The residue was purified by prep-TLC (SiO2, DCM:MeOH=10:1) to give the crude product, which was then further purified by Prep-HPLC (column: Waters Xbridge 150×25 mm×5 μm; mobile phase: [water (NH4HCO3)-ACN]; B %: 26%-56%, 9 min) to afford Compound 162 (6.34 mg, 14% yield) as a white solid.

LCMS: Rt=0.850 min; (ESI positive ion) m/z: 503.2 (M+H)+(calculated: 503.11).

1H NMR (400 MHz, MeOD-d4) δ=8.66 (s, 1H), 7.60 (d, J=8.4 Hz, 2H), 7.17 (d, J=8.3 Hz, 2H), 7.02-6.64 (m, 1H), 6.08 (d, J=4.8 Hz, 1H), 5.64 (s, 2H), 4.64 (br s, 1H), 4.38 (t, J=4.6 Hz, 1H), 4.22 (q, J=3.5 Hz, 1H), 3.85-3.74 (m, 2H), 3.73-3.70 (m, 2H), 3.63 (br dd, J=3.9, 5.1 Hz, 2H).

### Compound 163:

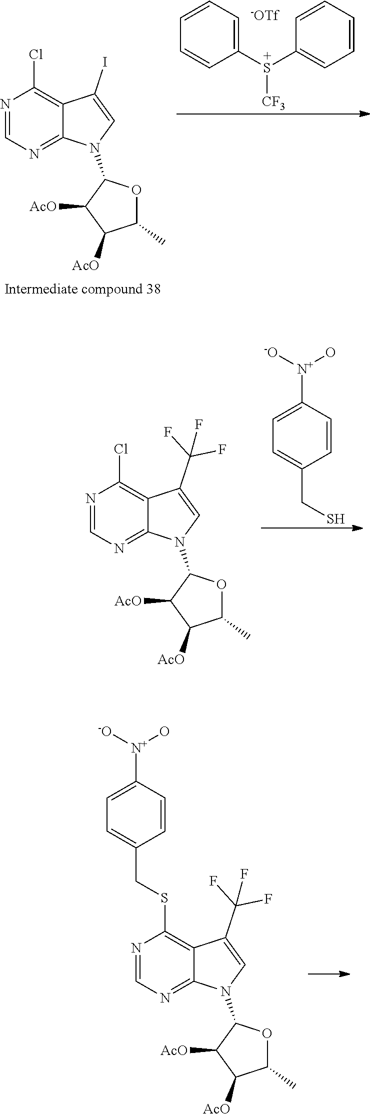

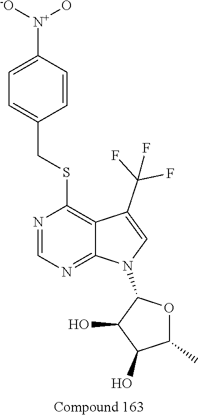

To a mixture of Intermediate Compound 38 (200 mg, 416.97 μmol, 1 eq) and S-(Trifluoromethyl)diphenylsulfonium trifluoromethanesulfonate (337.20 mg, 833.94 μmol, 2 eq) in DMF (10 mL) was added copper (79.49 mg, 1.25 mmol, 8.87 μL, 3 eq). The mixture was stirred at 60° C. for 16 hr. The mixture was then filtered and concentrated under vacuum. The residue was purified by Prep-TLC (Petroleum ether/Ethyl acetate=3/1, Rf=0.4). The purified solution was concentrated to afford the corresponding trifluoromethylated compound (82 mg, 47% yield) as a yellow oil.

LCMS: Rt=0.983 min; (ESI positive ion) m/z: 422.0 (M+H)+(calculated: 422.07).

To a solution of the above trifluoromethylated compound (81 mg, 192.05 μmol, 1 eq) and (4-nitrophenyl)methanethiol (129.98 mg, 768.22 μmol, 4 eq) in EtOH (2 mL) was added TEA (97.17 mg, 960.27 μmol, 133.66 μL, 5 eq). The mixture was stirred at 80° C. for 2 hr and then filtered and concentrated under vacuum. The residue was purified by Prep-TLC (Petroleum ether/Ethyl acetate=3/1, RF=0.3) to afford the corresponding thioether compound (107 mg, crude) as a yellow oil.

LCMS: Rt=1.068 min; (ESI positive ion) m/z: 555.2 (M+H)+(calculated: 555.11).

To a solution of the above thioether compound (80 mg, 144.28 μmol, 1 eq) in MeOH (2 mL) was added NH3·H2O (1.82 g, 14.54 mmol, 2.00 mL, 28% purity, 100.79 eq). The mixture was stirred at 20° C. for 0.5 hr and then concentrated to give a residue. The residue was purified by Prep-HPLC (column: Waters Xbridge 150×25 mm×5 μm; mobile phase: [water (NH4HCO3)-ACN]; B %: 45%-75%, 9 min). The purified solution was lyophilized to afford Compound 163 (13.65 mg, 20% yield) as a white solid.

LCMS: Rt=0.971 min; (ESI positive ion) m/z: 471.0 (M+H)+(calculated: 471.09).

1H NMR (400 MHz, DMSO-d6) δ=8.82 (s, 1H), 8.43 (s, 1H), 8.17 (d, J=8.9 Hz, 2H), 7.76 (d, J=8.8 Hz, 2H), 6.15 (d, J=5.6 Hz, 1H), 5.43 (d, J=6.0 Hz, 1H), 5.19 (d, J=5.1 Hz, 1H), 4.84-4.76 (m, 2H), 4.58 (q, J=5.6 Hz, 1H), 4.02-3.95 (m, 1H), 3.91 (q, J=4.9 Hz, 1H), 1.31 (d, J=6.4 Hz, 3H).

### Compound 165:

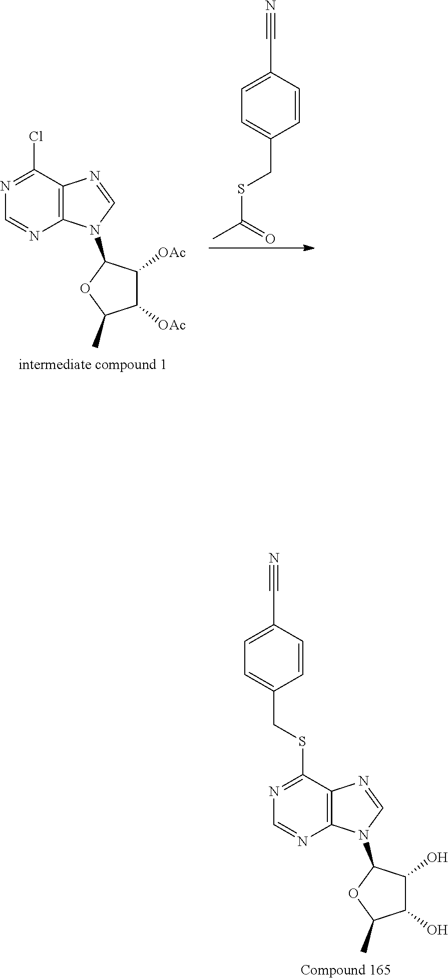 [SMILES: CC(=O)O[C@@H]1[C@H](OC(C)=O)[C@@H](C)O[C@H]1n1cnc2c(Cl)ncnc21 ; CC(=O)SCc1ccc(C#N)cc1 ; C[C@H]1O[C@@H](n2cnc3c(SCc4ccc(C#N)cc4)ncnc32)[C@H](O)[C@@H]1O]

To a solution of Intermediate Compound 1 (150 mg, 422.84 μmol, 1 eq) in THE (0.4 mL) and MeOH (1.6 mL) was added S-(4-cyanobenzyl) ethanethioate (88.95 mg, 465.12 mol, 1.1 eq). The mixture was cooled to 0° C., then K2CO3 (122.72 mg, 887.96 μmol, 2.1 eq) was added. The mixture was stirred at 0° C. for 1 h. The residue was dissolved in MeCN, and then purified by prep-HPLC (column: Unisil 3-100 C18 Ultra 150×50 mm×3 μm; mobile phase: [water(0.225% FA)-ACN]) and lyophilized to give the Compound 165 (30.8 mg, 19% yield) as a white solid.

LCMS: Rt=0.821 min; (ESI positive ion) m/z: 384.0 (M+H)+(calculated: 384.11).

1H NMR (400 MHz, CDCl₃) δ=8.69 (s, 1H), 8.24 (s, 1H), 7.59 (s, 4H), 5.92 (d, J=5.50 Hz, 1H), 4.74-4.64 (m, 2H), 4.63-4.59 (m, 1H), 4.47-4.39 (m, 1H), 4.20 (dd, J=5.14, 2.88 Hz, 1H), 1.40 (d, J=6.4 Hz, 3H).

### Compound 166:

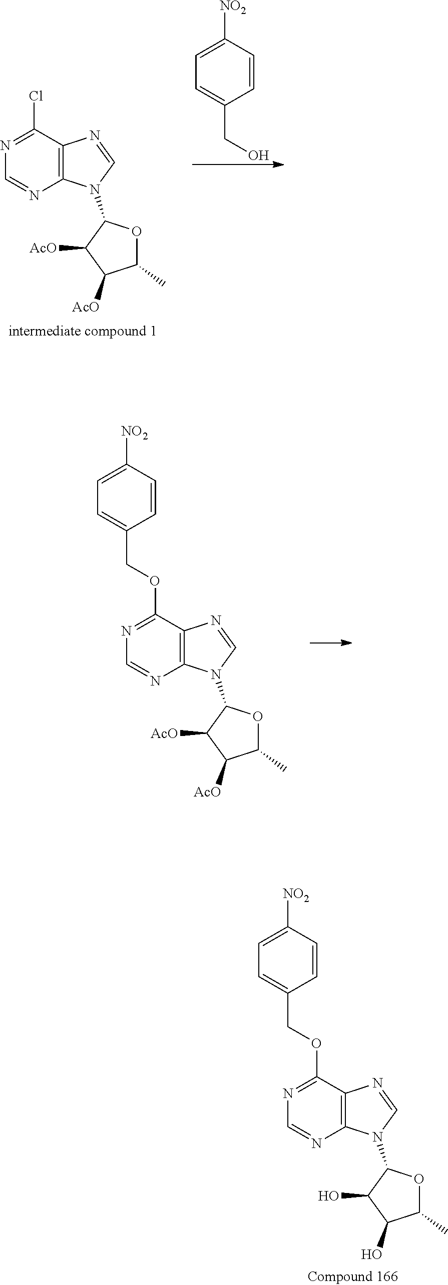 [SMILES: CC(=O)O[C@@H]1[C@H](OC(C)=O)[C@@H](C)O[C@H]1n1cnc2c(Cl)ncnc21 ; O=[N+]([O-])c1ccc(CO)cc1 ; CC(=O)O[C@@H]1[C@H](OC(C)=O)[C@@H](C)O[C@H]1n1cnc2c(OCc3ccc([N+](=O)[O-])cc3)ncnc21 ; C[C@H]1O[C@@H](n2cnc3c(OCc4ccc([N+](=O)[O-])cc4)ncnc32)[C@H](O)[C@@H]1O]

To a solution of Intermediate Compound 1 (4 g, 11.28 mmol, 1 eq) in toluene (20 mL) was added (4-nitrophenyl)methanol (2.07 g, 13.53 mmol, 1.2 eq), Cs2CO3 (9.18 g, 28.19 mmol, 2.5 eq), Pd2(dba)3 (1.03 g, 1.13 mmol, 0.1 eq) and Xantphos (1.30 g, 2.26 mmol, 0.2 eq). The mixture was degassed and purged with N2 3 times and stirred at 80° C. for 2 hr under N2 atmosphere. The reaction mixture was filtered and concentrated under reduce pressure to give a residue. The residue was purified by column chromatography (SiO2, Petroleum ether/Ethyl acetate=5/1 to 1/1) to afford the corresponding ether (3.9 g, 26% yield) as a yellow solid.

LCMS: Rt=0.541 min; (ESI positive ion) m/z: 413.1 (M+H)+(calculated: 472.14).

To a solution of the above ether (3.9 g, 8.27 mmol, 1 eq) in MeOH (40 mL) was added NH3·H2O (50.83 g, 406.12 mmol, 55.86 mL, 28% purity). The mixture was stirred at 20° C. for 1 hr. The reaction mixture was filtered and concentrated under reduce pressure to give a residue. The residue was purified by Prep-HPLC (column: Phenomenex luna C18 250×50 mm×15 μm; mobile phase: [water (NH4HCO3)-ACN]; B %: 18%-48%, 20 min) to afford Compound 166 (1.10 g, 34.30% yield) as a white solid.

LCMS: Rt=0.439 min; (ESI positive ion) m/z: 388.1 (M+H)+(calculated: 388.12).

1H NMR (400 MHz, DMSO-d6) δ=8.64 (s, 1H), 8.56 (s, 1H), 8.27 (d, J=8.6 Hz, 2H), 7.76 (d, J=8.6 Hz, 2H), 5.95 (d, J=5.0 Hz, 1H), 5.80 (s, 2H), 5.48 (d, J=5.6 Hz, 1H), 5.21 (d, J=4.8 Hz, 1H), 4.70 (q, J=5.0 Hz, 1H), 4.03-3.97 (m, 2H), 1.32 (d, J=5.8 Hz, 3H).

### Compound 168:

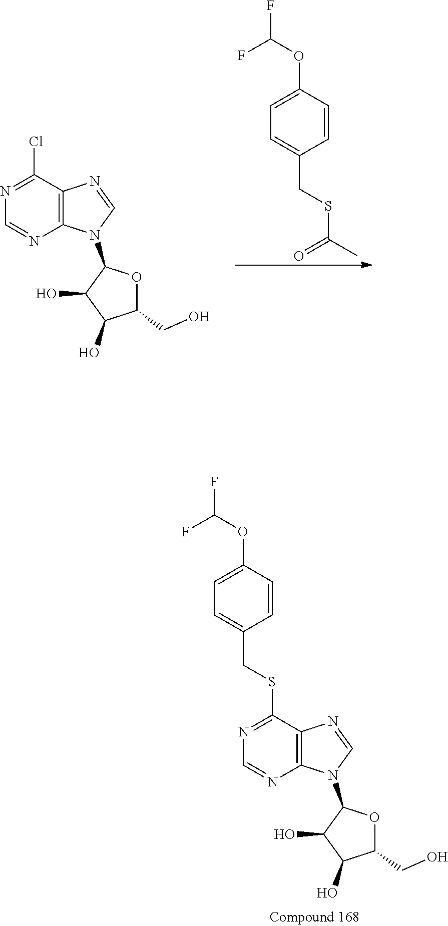 [SMILES: OC[C@H]1O[C@H](n2cnc3c(Cl)ncnc32)[C@H](O)[C@@H]1O ; CC(=O)SCc1ccc(OC(F)F)cc1 ; OC[C@H]1O[C@H](n2cnc3c(SCc4ccc(OC(F)F)cc4)ncnc32)[C@H](O)[C@@H]1O]

To a solution of (2R,3R,4S,5R)-2-(6-chloro-9H-purin-9-yl)-5-(hydroxymethyl)tetrahydrofuran-3,4-diol (150 mg, 523.25 μmol, 1 eq) and S-(4-(difluoromethoxy)benzyl) ethanethioate (145.83 mg, 627.90 μmol, 1.2 eq) in MeOH (2 mL) in THE (0.5 mL) was added K2CO3 (144.63 mg, 1.05 mmol, 2 eq) in one portion at 0° C. The mixture was stirred at 0° C. for 0.5 hours and then filtered and concentrated under vacuum. The resultant residue was purified by Prep-HPLC (column: Unisil 3-100 C18 Ultra 150×50 mm×3 m; mobile phase: [water(FA)-ACN]; B %: 25%-45%, 10 min) to afford Compound 168 (121.32 mg, 53% yield) as a white solid.

LCMS: Rt=0.832 min; (ESI positive ion) m/z: 441.1 (M+H)+(calculated: 441.10).

1H NMR (400 MHz, DMSO-d6) δ=8.79 (s, 1H), 8.72 (s, 1H), 7.54-7.50 (m, 2H), 7.38-7.00 (m, 3H), 5.99 (d, J=5.6 Hz, 1H), 5.52 (d, J=5.8 Hz, 1H), 5.23 (d, J=5.0 Hz, 1H), 5.10 (t, J=5.6 Hz, 1H), 4.67 (d, J=1.4 Hz, 2H), 4.59 (q, J=5.4 Hz, 1H), 4.20-4.15 (m, 1H), 3.97 (q, J=3.8 Hz, 1H), 3.68 (td, J=4.6, 12.0 Hz, 1H), 3.57 (ddd, J=4.0, 6.0, 12.0 Hz, 1H).

### Compound 169:

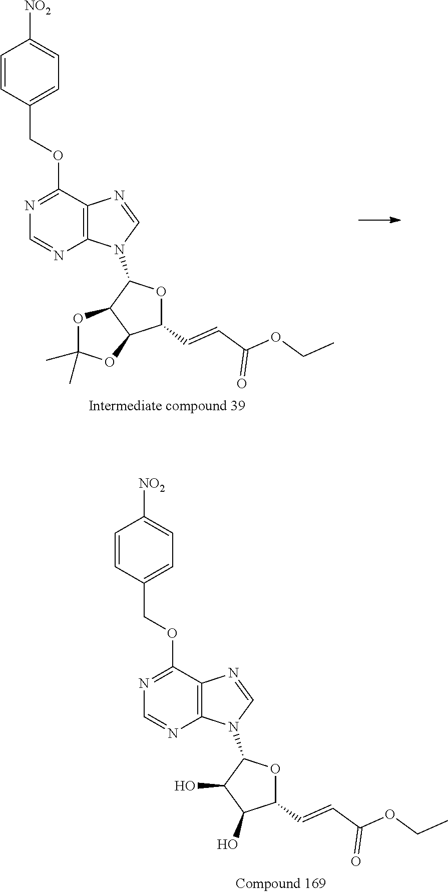 [SMILES: CCOC(=O)/C=C/[C@H]1O[C@@H](n2cnc3c(OCc4ccc([N+](=O)[O-])cc4)ncnc32)[C@@H]2OC(C)(C)O[C@@H]21 ; CCOC(=O)/C=C/[C@H]1O[C@@H](n2cnc3c(OCc4ccc([N+](=O)[O-])cc4)ncnc32)[C@H](O)[C@@H]1O]

To a solution of Intermediate Compound 39 (80 mg, 156.41 μmol, 1 eq) in water (0.5 mL) was added formic acid (7.51 mg, 156.41 μmol, 2 mL, 1 eq) at 0° C. The mixture was stirred at 25° C. for 1.5 hr. The reaction mixture was then quenched by the addition of a saturated aqueous solution of NaHCO3 (20 mL) until a pH of about 8 at 0° C. was reached. The mixture was then extracted with EtOAc (30 mL×2). The combined organic layers were washed with brine (30 mL), dried over Na2SO4, filtered and concentrated under vacuum. The resultant residue was purified by Prep-HPLC (column: Waters xbridge 150×25 mm×10 μm; mobile phase: [water (NH4HCO3)-ACN]; B %: 30%-60%, 10 min) to afford Compound 169 (26.79 mg, 36% yield) as a white solid.

LCMS: Rt=0.856 min; (ESI positive ion) m/z: 471.9 (M+H)+(calculated: 472.14).

1H NMR (400 MHz, DMSO-d6) δ=8.68 (s, 1H), 8.57 (s, 1H), 8.33-8.25 (m, 2H), 7.77 (d, J=8.8 Hz, 2H), 7.06 (dd, J=5.8, 15.7 Hz, 1H), 6.11-5.99 (m, 2H), 5.81 (s, 2H), 5.73-5.57 (m, 2H), 4.76 (q, J=4.8 Hz, 1H), 4.64-4.54 (m, 1H), 4.30 (q, J=5.2 Hz, 1H), 4.14 (q, J=7.2 Hz, 2H), 1.22 (t, J=7.2 Hz, 3H).

### Compound 172:

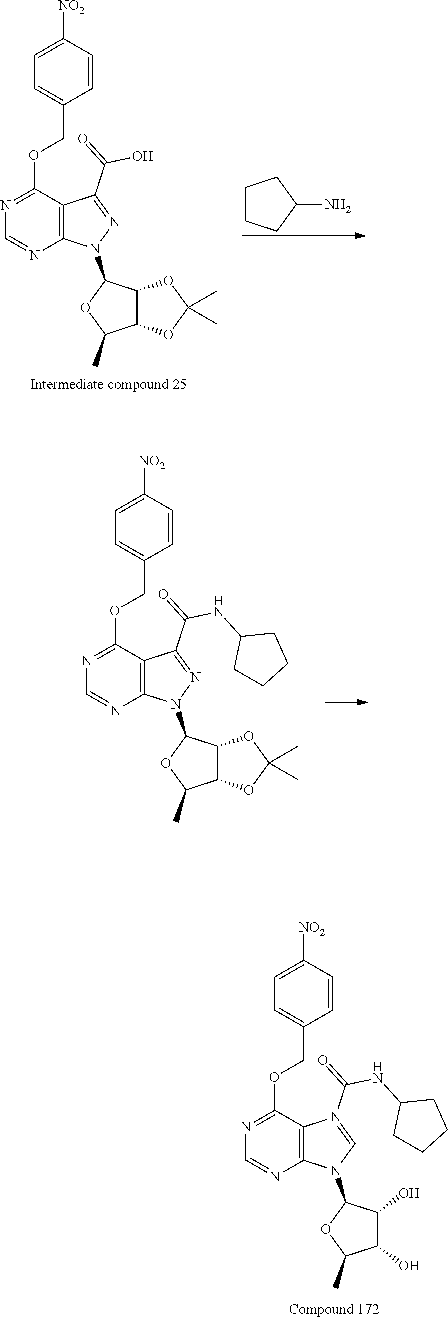

To a solution of Intermediate Compound 25 (12 mg, 25.46 μmol, 1 eq) in THE (1 mL) was added cyclopentanamine (4.77 mg, 56.00 μmol, 2.2 eq), DIEA (19.74 mg, 152.73 mol, 6 eq) and T3P (48.60 mg, 76.37 μmol, 3 eq) at 0° C. The mixture was stirred at 50° C. for 2 hr. The reaction mixture was then filtered and filtrate was concentrated under reduced pressure to give a residue. The residue was purified by Prep-TLC (SiO2, Petroleum/Ethyl acetate=1/1) to afford the corresponding amide (11 mg, 80% yield) as a colorless oil.

LCMS: Rt=0.548 min; (ESI positive ion) m/z: 539.4 (M+H)+(calculated: 539.22).

A solution of the above amide (11 mg, 20.43 μmol, 1 eq) in HCOOH (0.8 mL) and water (0.2 mL) was stirred at 0° C. for 6 hr. The reaction mixture was then filtered and the filtrate was purified by Prep-TLC (SiO2, DCM/MeOH=10/1) to afford Compound 172 (3.6 mg, 35% yield) as a white solid.

LCMS: Rt=0.446 min; (ESI positive ion) m/z: 499.1 (M+H)+(calculated: 499.19).

1H NMR (400 MHz, DMSO-d6) δ=8.70 (s, 1H), 8.40 (br d, J=7.6 Hz, 1H), 8.27 (br d, J=8.6 Hz, 2H), 7.85 (br d, J=8.6 Hz, 2H), 6.22 (d, J=3.8 Hz, 1H), 5.79 (s, 2H), 5.49 (br d, J=5.2 Hz, 1H), 5.21 (br d, J=5.4 Hz, 1H), 4.68-4.64 (m, 1H), 4.22-4.17 (m, 1H), 4.09 (q, J=5.0 Hz, 1H), 4.04-4.00 (m, 1H), 1.84-1.79 (m, 2H), 1.54-1.46 (m, 4H), 1.34 (br dd, J=5.0, 7.2 Hz, 2H), 1.25 (br d, J=6.4 Hz, 3H).

### Compound 175:

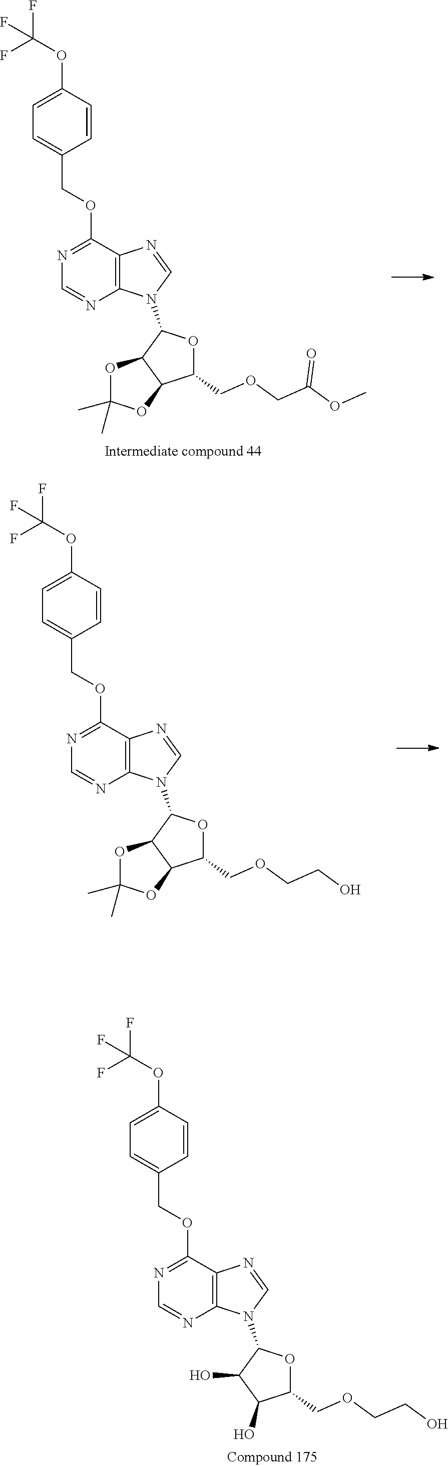 [SMILES: COC(=O)COC[C@H]1O[C@@H](n2cnc3c(OCc4ccc(OC(F)(F)F)cc4)ncnc32)[C@@H]2OC(C)(C)O[C@@H]21 ; CC1(C)O[C@@H]2[C@H](O1)[C@@H](COCCO)O[C@H]2n1cnc2c(OCc3ccc(OC(F)(F)F)cc3)ncnc21 ; OCCOC[C@H]1O[C@@H](n2cnc3c(OCc4ccc(OC(F)(F)F)cc4)ncnc32)[C@H](O)[C@@H]1O]

To a solution of Intermediate Compound 44 (240 mg, 432.84 μmol, 1 eq) in THE (5 mL) was added LiBH4 (2 M, 324.63 μL, 1.5 eq) at 0° C. The mixture was stirred at 0° C. for 2 hr. The mixture was then added to ice-water (20 mL) dropwise at 0° C. and stirred for 5 min. The mixture was extracted with EtOAc (10 mL×2). The combined organic phase was washed with brine (20 mL), dried with anhydrous Na2SO4, filtered and concentrated under vacuum. The resultant residue was purified by Prep-TLC (Petroleum ether/Ethyl acetate=1/3) to afford the corresponding alcohol compound (110 mg, 48% yield) as a colorless oil.

A solution of the above alcohol compound (110 mg, 208.94 μmol, 1 eq) in formic acid (4 mL) and water (1 mL) was stirred at 20° C. for 1 hr. The mixture was concentrated under vacuum and the resultant residue was purified by Prep-TLC (DCM/MeOH=10/1) to afford Compound 175 (22.9 mg, 22% yield) as a white solid.

LCMS: Rt=0.853 min; (ESI positive ion) m/z: 487.1 (M+H)+(calculated: 487.14).

1H NMR (400 MHz, DMSO-d6) δ=8.67 (s, 1H), 8.58 (s, 1H), 7.65 (d, J=8.8 Hz, 2H), 7.41 (d, J=8.0 Hz, 2H), 6.02 (d, J=5.6 Hz, 1H), 5.67 (s, 2H), 5.54 (d, J=6.0 Hz, 1H), 5.31 (d, J=5.2 Hz, 1H), 4.68 (t, J=5.2 Hz, 1H), 4.61 (q, J=5.4 Hz, 1H), 4.21-4.16 (m, 1H), 4.06 (q, J=4.0 Hz, 1H), 3.70-3.65 (m, 1H), 3.63-3.58 (m, 1H), 3.55-3.51 (m, 2H), 3.49-3.45 (m, 2H).

### Compound 177:

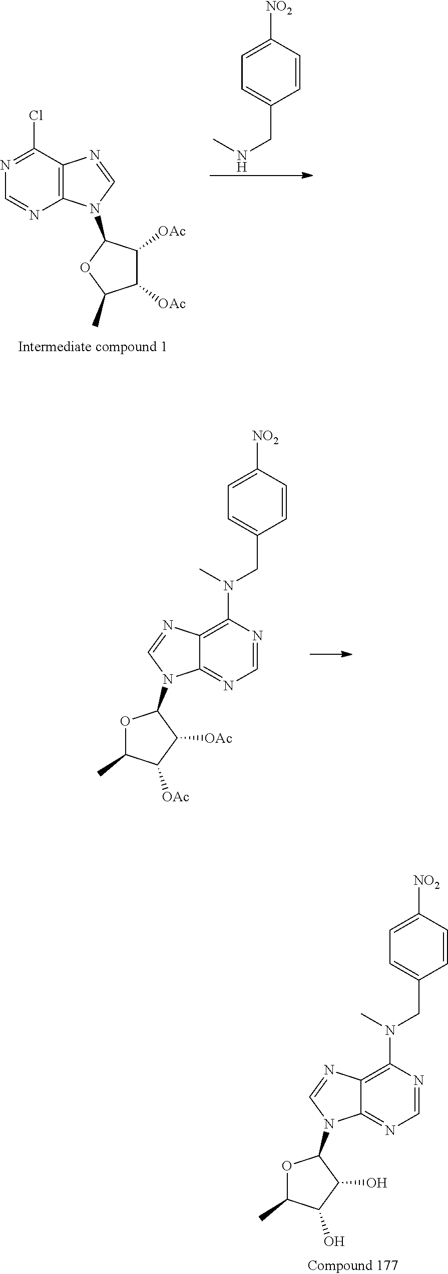 [SMILES: CC(=O)O[C@@H]1[C@H](OC(C)=O)[C@@H](C)O[C@H]1n1cnc2c(Cl)ncnc21 ; CNCc1ccc([N+](=O)[O-])cc1 ; CC(=O)O[C@@H]1[C@H](OC(C)=O)[C@@H](C)O[C@H]1n1cnc2c(N(C)Cc3ccc([N+](=O)[O-])cc3)ncnc21 ; C[C@H]1O[C@@H](n2cnc3c(N(C)Cc4ccc([N+](=O)[O-])cc4)ncnc32)[C@H](O)[C@@H]1O]

To a solution of Intermediate Compound 1 (200 mg, 563.79 μmol, 1 eq) in ethanol (2 mL) was added TEA (285.24 mg, 2.82 mmol, 5 eq) and N-methyl-1-(4-nitrophenyl)methanamine (187.38 mg, 1.13 mmol, 2 eq). The mixture was stirred at 80° C. for 1 hr. The mixture was then concentrated under reduce pressure to give the corresponding amine compound (300 mg, crude), which was used in the next step without further purification.

To a solution of the above crude material (300 mg, 619.24 μmol, 1 eq) in MeOH (3 mL) was added NH3·H2O (4.55 g, 129.81 mmol, 5 mL, 209.63 eq). The mixture was stirred at 50° C. for 0.5 hr. The mixture was then concentrated under reduced pressure to give a residue. The residue was dissolved in MeOH and purified by Prep-HPLC (Column: Phenomenex Gemini-NX C18 75×30 mm×3 um, Mobile phase: [water(0.225% FA)-ACN]; B %: 25%-55%, 7 min, Wavelength: 220&254 nm) and lyophilized to give Compound 177 as a white solid. LCMS: Rt=0.806 min; (ESI positive ion) m/z: 401.2 (M+H)+(calculated: 401.15).

1H NMR (400 MHz, CDCl₃) δ=8.35 (s, 1H), 8.20 (d, J=8.4 Hz, 2H), 7.97 (s, 1H), 7.46 (d, J=8.8 Hz, 2H), 5.96 (d, J=5.26 Hz, 1H), 5.80-4.91 (m, 2H), 4.57-4.35 (m, 2H), 4.19-4.05 (m, 1H), 3.68-3.15 (m, 3H), 1.40 (d, J=6.8 Hz, 3H)

### Compound 183:

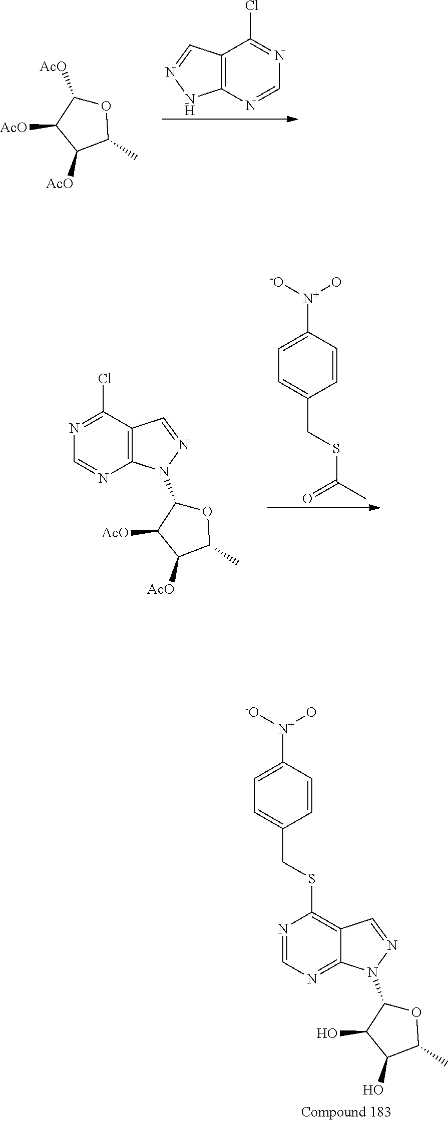

To a solution of 4-chloro-1H-pyrazolo[3,4-d]pyrimidine (285.07 mg, 1.84 mmol, 1.2 eq) in MeCN (5 mL) was added Bis(trimethylsilyl)acetamide (625.36 mg, 3.07 mmol, 759.85 μL, 2 eq). The mixture was stirred at 20° C. for 2 hr. After 2 hr, the mixture was concentrated to give a residue. A solution of (2S,3R,4R,5R)-5-methyltetrahydrofuran-2,3,4-triyl triacetate (400 mg, 1.54 mmol, 1 eq) in MeCN (5 mL) was added to the residue. SnCl4 (1.20 g, 4.61 mmol, 538.70 μL, 3 eq) was then added to the mixture dropwise at 0° C. and the mixture was stirred at 20° C. for 12 hr. The reaction mixture was slowly poured into a saturated aqueous NaHCO3 (40 mL) and extracted with EtOAc (30 mL×2). The organic layer was washed with brine, dried by Na2SO4, and concentrated to give a residue. The residue was purified by Prep-TLC (SiO2, PE:EtOAc=3:1) to afford the conjugated intermediate compound (107 mg, 20% yield) as a colorless oil.

LCMS: Rt=0.873 min; (ESI positive ion) m/z: 355.1 (M+H)+(calculated: 355.07).

1H NMR (400 MHz, CDCl₃) δ=8.82 (s, 1H), 8.25 (s, 1H), 6.58 (d, J=3.6 Hz, 1H), 6.02 (dd, J=3.8, 5.3 Hz, 1H), 5.51 (t, J=5.6 Hz, 1H), 4.37 (quin, J=6.3 Hz, 1H), 2.15 (s, 3H), 2.10 (s, 3H), 1.44 (d, J=6.4 Hz, 3H).

A solution of the above conjugated intermediate compound (100 mg, 281.89 μmol, 1 eq) and S-(4-nitrobenzyl) ethanethioate (71.46 mg, 338.27 μmol, 1.2 eq) in MeOH (2 mL) and THE (0.5 mL) was stirred at 0° C. while K2CO3 (81.81 mg, 591.97 μmol, 2.1 eq) was added in one portion. The mixture was stirred at 0° C. for 2 hr. The reaction mixture was added to MeOH (20 mL) and filtered. The filtrate was concentrated to give a residue, which was purified by Prep-IPLC (column: Phenomenex C18 75×30 mm×3 μm; mobile phase: [water(FA)-ACN]; B %: 32%-62%, 7 min) and lyophilized. The resultant residue was purified by Prep-TLC (SiO2, PE:EtOAc=1:1) to afford Compound 183 (22.75 mg, 20% yield) as a white solid.

LCMS: Rt=0.864 min; (ESI positive ion) m/z: 404.1 (M+H)+(calculated: 404.10).

1H NMR (400 MHz, DMSO-d6) δ=8.85 (s, 1H), 8.46 (s, 1H), 8.18 (d, J=8.7 Hz, 2H), 7.76 (d, J=8.7 Hz, 2H), 6.19 (d, J=3.7 Hz, 1H), 5.45 (br s, 1H), 5.17 (br d, J=5.0 Hz, 1H), 4.83 (s, 2H), 4.57 (br s, 1H), 4.08 (br d, J=4.9 Hz, 1H), 4.00 (quin, J=6.0 Hz, 1H), 1.22 (d, J=6.4 Hz, 3H).

### Compound 188:

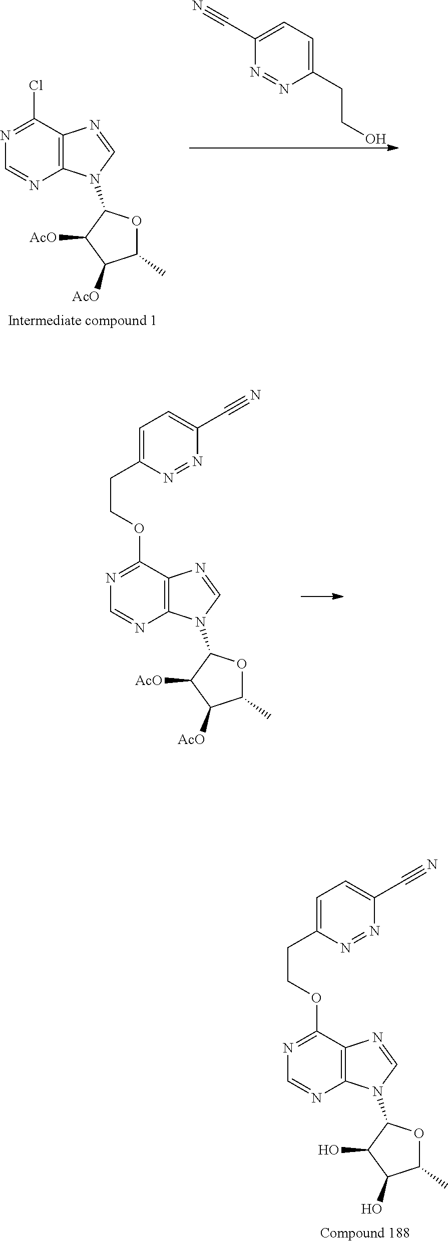 [SMILES: CC(=O)O[C@@H]1[C@H](OC(C)=O)[C@@H](C)O[C@H]1n1cnc2c(Cl)ncnc21 ; N#Cc1ccc(CCO)nn1 ; CC(=O)O[C@@H]1[C@H](OC(C)=O)[C@@H](C)O[C@H]1n1cnc2c(OCCc3ccc(C#N)nn3)ncnc21 ; C[C@H]1O[C@@H](n2cnc3c(OCCc4ccc(C#N)nn4)ncnc32)[C@H](O)[C@@H]1O]

To a solution of Intermediate Compound 1 (40 mg, 112.76 μmol, 1 eq) and 6-(2-hydroxyethyl)pyridazine-3-carbonitrile (18.50 mg, 124.03 μmol, 1.1 eq) in toluene (2 mL) was added Cs2CO3 (91.85 mg, 281.89 μmol, 2.5 eq), Xantphos (13.05 mg, 22.55 μmol, 0.2 eq) and Pd2(dba)3 (10.33 mg, 11.28 μmol, 0.1 eq). The mixture was stirred at 80° C. for 0.5 hr and then filtered through a pad of silica gel. The filtrate was evaporated under reduced pressure to give a residue, which was purified by Prep-TLC (SiO2, Petroleum ether/Ethyl acetate=0/1). The purified solution was concentrated to afford the corresponding ether compound (20 mg, 38% yield) as a yellow oil.

LCMS: Rt=0.827 min; (ESI positive ion) m/z: 468.1 (M+H)+(calculated: 468.16).

To a solution of the above ether compound (15 mg, 32.09 μmol, 1 eq) in THE (1 mL) was added NH3·H2O (910.00 mg, 7.27 mmol, 1 mL, 28% purity, 226.56 eq). The mixture was stirred at 20° C. for 2 hr and then concentrated to give a residue. The residue was purified by Prep-HPLC (column: Phenomenex C18 75×30 mm×3 μm; mobile phase: [water(FA)-ACN]; B %: 10%-40%, 7 min) followed by a Prep-TLC (SiO2, DCM:MeOH=10:1, Rf=0.5) to afford Compound 188 (2 mg, 15% yield) as a white solid.

LCMS: Rt=0.703 min; (ESI positive ion) m/z: 384.1 (M+H)+(calculated: 384.14).

1H NMR (400 MHz, MeOD-d4) δ=8.51 (s, 1H), 8.40 (s, 1H), 8.10-8.06 (m, 1H), 8.01-7.97 (m, 1H), 6.01 (d, J=4.5 Hz, 1H), 5.10 (t, J=6.2 Hz, 2H), 4.77 (t, J=4.7 Hz, 1H), 4.17-4.09 (m, 2H), 3.68 (t, J=6.2 Hz, 2H), 1.42 (d, J=6.2 Hz, 3H).

### Compound 189:

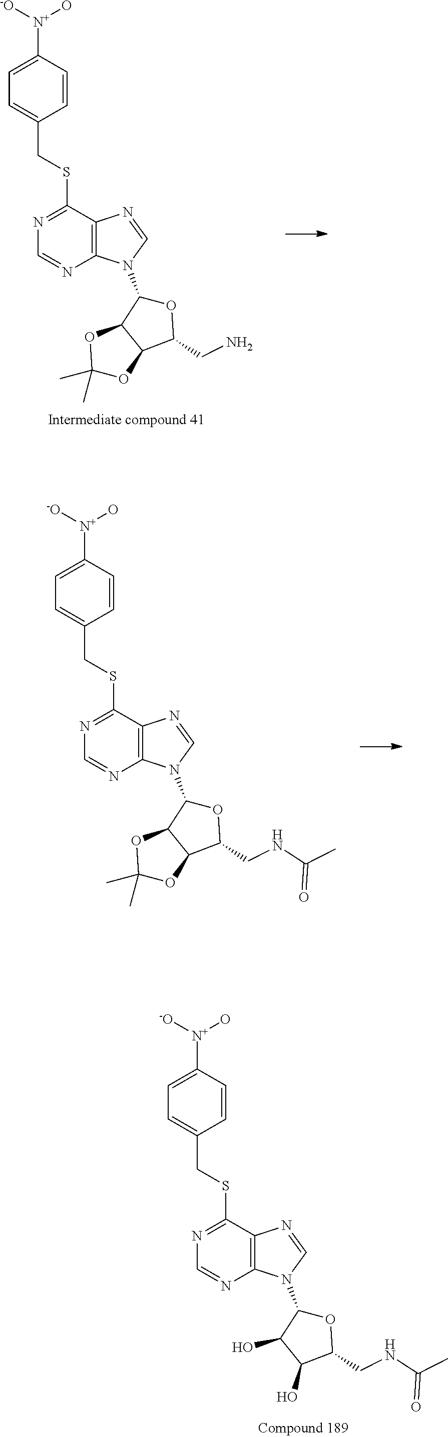

To a solution of Intermediate Compound 41 (50 mg, 109.05 μmol, 1 eq) and TEA (22.07 mg, 218.11 μmol, 30.36 μL, 2 eq) in DCM (1 mL) was added acetic anhydride (11.13 mg, 109.05 μmol, 10.21 μL, 1 eq) at 0° C. The mixture was stirred at 20° C. for 1 hour and then poured into water (30 mL). The aqueous phase was extracted with DCM (30 mL×2). The combined organic phase was washed with brine (20 mL), dried with anhydrous Na2SO4, filtered and concentrated under vacuum to afford the corresponding acetylated compound (50 mg, crude).

LCMS: Rt=0.891 min; (ESI positive ion) m/z: 501.0 (M+H)+(calculated: 501.15).

To a mixture of the above acetylated compound (60 mg, 119.87 μmol, 1 eq) in water (0.5 mL) was added TFA (770.00 mg, 6.75 mmol, 0.5 mL, 56.34 eq) at 0° C. The mixture was stirred at 20° C. for 2 hours. The reaction mixture was then filtered and concentrated under reduced pressure to give a residue. The residue was purified by Prep-HPLC (column: Phenomenex Gemini-NX C18 75×30 mm×3 μm; mobile phase: [water(0.225% FA)-ACN]; B %: 22%-52%, 7 min) to afford Compound 189 (25.08 mg, 45% yield) as a white solid.

LCMS: Rt=0.813 min; (ESI positive ion) m/z: 461.3 (M+H)+(calculated: 461.13).

1H NMR (400 MHz, DMSO-d6) δ=8.90-8.64 (m, 2H), 8.18-8.01 (m, 2H), 7.88-7.66 (m, 2H), 5.96 (br d, J=3.3 Hz, 1H), 5.55-5.27 (m, 1H), 4.93-4.57 (m, 3H), 4.21-3.88 (m, 2H), 3.52-3.34 (m, 4H), 1.89-1.76 (m, 3H).

### Compound 190:

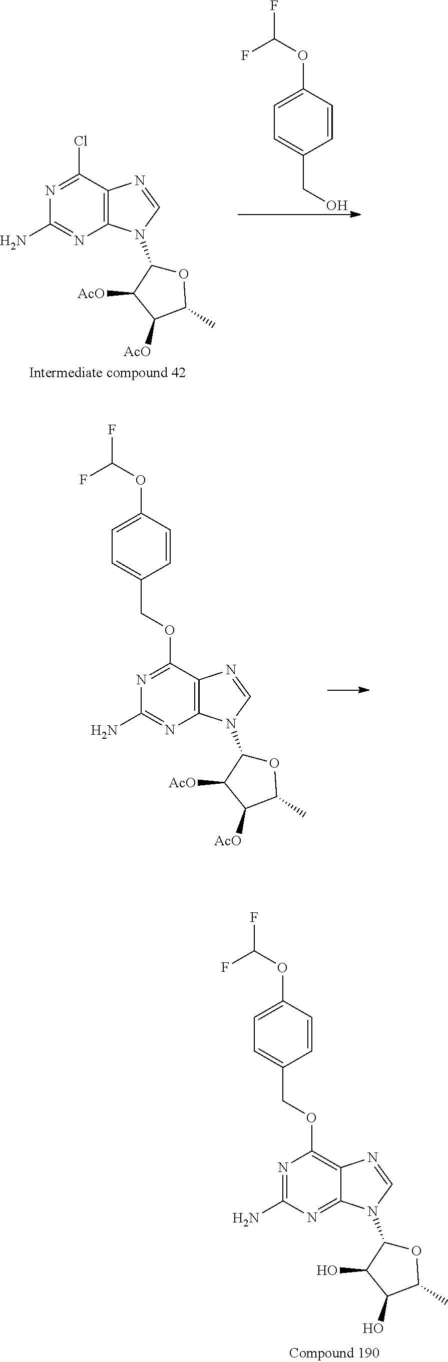 [SMILES: CC(=O)O[C@@H]1[C@H](OC(C)=O)[C@@H](C)O[C@H]1n1cnc2c(Cl)nc(N)nc21 ; OCc1ccc(OC(F)F)cc1 ; CC(=O)O[C@@H]1[C@H](OC(C)=O)[C@@H](C)O[C@H]1n1cnc2c(OCc3ccc(OC(F)F)cc3)nc(N)nc21 ; C[C@H]1O[C@@H](n2cnc3c(OCc4ccc(OC(F)F)cc4)nc(N)nc32)[C@H](O)[C@@H]1O]

Compound 190 was synthesized using the protocols described for Compound 161 from Intermediate Compound 42 and (4-(difluoromethoxy)phenyl)methanol.

LCMS: Rt=0.815 min; (ESI positive ion) m/z: 424.1 (M+H)+(calculated: 424.14).

1H NMR (400 MHz, DMSO-d6) δ=8.05 (s, 1H), 7.57 (d, J=8.6 Hz, 2H), 7.24 (t, J=74.0 Hz, 1H), 7.19 (d, J=8.4 Hz, 2H), 6.51 (s, 2H), 5.73 (d, J=5.3 Hz, 1H), 5.48 (s, 2H), 5.47-5.35 (m, 1H), 5.12 (br s, 1H), 4.58-4.51 (m, 1H), 3.97-3.86 (m, 2H), 1.28 (d, J=6.1 Hz, 3H).

### Compound 191:

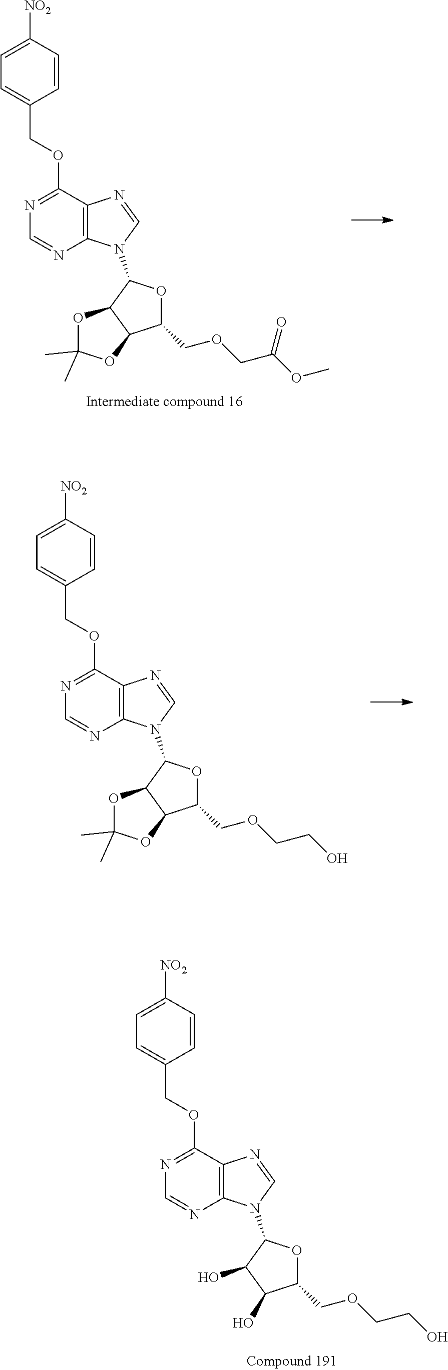 [SMILES: COC(=O)COC[C@H]1O[C@@H](n2cnc3c(OCc4ccc([N+](=O)[O-])cc4)ncnc32)[C@@H]2OC(C)(C)O[C@@H]21 ; CC1(C)O[C@@H]2[C@H](O1)[C@@H](COCCO)O[C@H]2n1cnc2c(OCc3ccc([N+](=O)[O-])cc3)ncnc21 ; O=[N+]([O-])c1ccc(COc2ncnc3c2ncn3[C@@H]2O[C@H](COCCO)[C@@H](O)[C@H]2O)cc1]

To a solution of Intermediate Compound 16 (500 mg, 969.99 μmol, 1 eq) in THE (5 mL) was added LiBH4 (2 M, 533.49 μL, 1.1 eq) at 0° C. under N2. The mixture was stirred at 0° C. for 1 hr. The mixture was then added to water (100 mL) dropwise at 0° C. and stirred for 5 min. The mixture was extracted with EtOAc (50 mL×2) and the combined organic phase was washed with brine (100 mL), dried with anhydrous Na2SO4, filtered and concentrated under vacuum. The residue was purified by Prep-TLC (Petroleum ether/Ethyl acetate=0/1) to afford the corresponding alcohol (250 mg, 53% yield) as a yellow oil.

LCMS: Rt=0.399 min; (ESI positive ion) m/z: 488.1 (M+H)+(calculated: 488.17).

A solution of the above alcohol (100 mg, 205.14 μmol, 1 eq) in HCOOH (0.8 mL) and H2O (0.2 mL) was stirred at 20° C. for 60 min. The mixture was concentrated under vacuum, and then the residue was treated with K2CO3 (2 mg)/MeOH (0.5 mL) and stirred at 20° C. for 10 min. The mixture was then filtered. The filtrate was purified by prep-TLC (DCM/MeOH=10/1) and then purified again by Prep-HPLC (column: Waters xbridge 150×25 mm 10 μm; mobile phase: [water (NH4HCO3)-can]; B %: 10%-40%, 11 min) to afford Compound 191 (27.27 mg, 29% yield) as a yellow solid.

LCMS: Rt=0.294 min; (ESI positive ion) m/z: 448.1 (M+H)+(calculated: 448.14).

1H NMR (400 MHz, DMSO-d6) δ=8.69 (s, 1H), 8.56 (s, 1H), 8.27 (br d, J=8.0 Hz, 2H), 7.76 (br d, J=8.0 Hz, 2H), 6.03 (br d, J=5.0 Hz, 1H), 5.80 (br s, 2H), 5.54 (br s, 1H), 5.31 (br s, 1H), 4.68 (br s, 1H), 4.62 (br s, 1H), 4.19 (br s, 1H), 4.07 (br d, J=2.8 Hz, 1H), 3.69-3.59 (m, 2H), 3.54-3.47 (m, 4H).

### Compound 192:

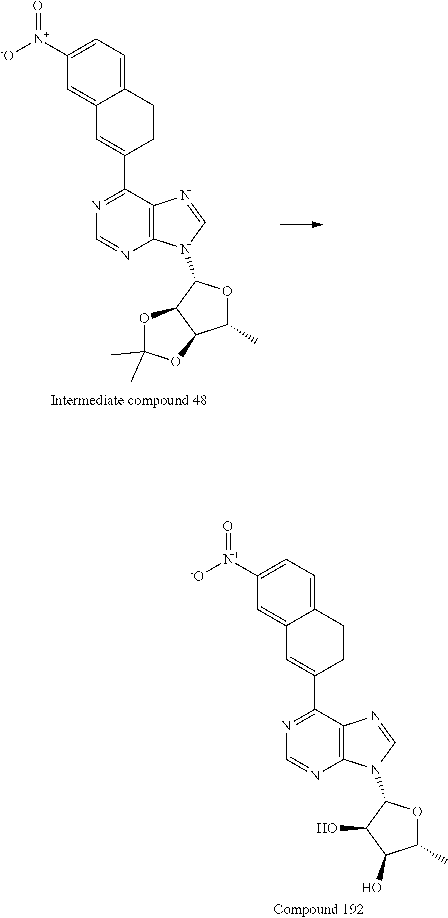

To a solution of the Intermediate Compound 48 (65 mg, 144.62 μmol, 1 eq) in water (0.4 mL) was added formic acid (6.95 mg, 144.62 μmol, 1.6 mL, 1 eq) at 0° C. The mixture was stirred at 25° C. for 1.5 hr and then quenched by addition of a saturated aqueous solution of NaHCO3 (20 mL) until a pH of about 8 at 0° C. was reached. The mixture was then extracted with EtOAc (30 mL×2). The combined organic layers were washed with brine (30 mL), dried over Na2SO4, filtered and concentrated under reduced pressure to give a residue. The residue was purified by Prep-HPLC (neutral, column: Waters Xbridge 150×25 mm×5 μm; mobile phase: [water (NH4HCO3)-ACN]; B %: 34%-64%, 10 min) to give Compound 192 (35.4 mg, 59% yield) as a white solid.

LCMS: Rt=0.856 min; (ESI positive ion) m/z: 409.9 (M+H)+(calculated: 410.14).

1H NMR (400 MHz, DMSO-d6) δ=8.98 (s, 1H), 8.87 (s, 1H), 8.63 (s, 1H), 8.19 (d, J=2.4 Hz, 1H), 8.14-8.11 (m, 1H), 7.56 (d, J=8.0 Hz, 1H), 6.04 (d, J=4.8 Hz, 1H), 5.55 (d, J=5.6 Hz, 1H), 5.26 (d, J=4.4 Hz, 1H), 4.75 (q, J=4.8 Hz, 1H), 4.09-4.01 (m, 2H), 3.16-3.09 (m, 4H), 1.36 (d, J=6.0 Hz, 3H).

### Compound 194 and Compound 286:

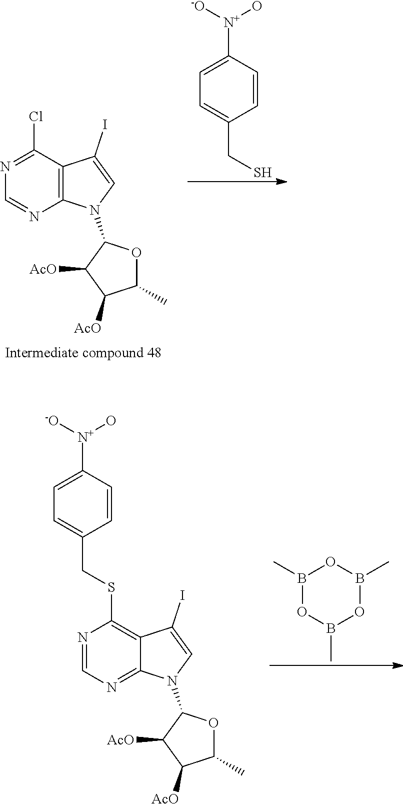

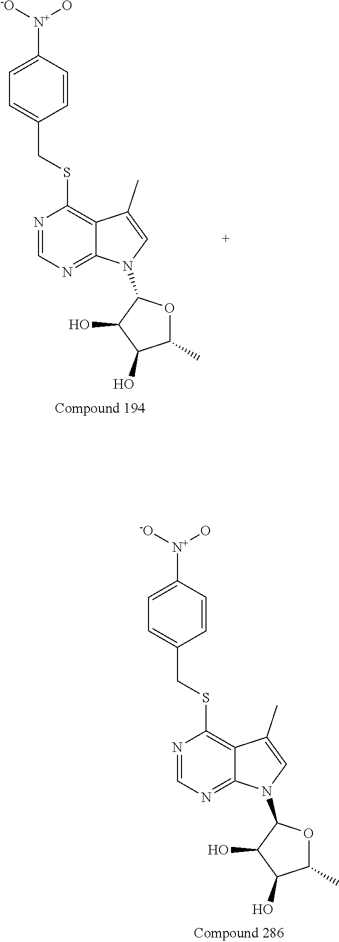

To a solution of Intermediate Compound 38 (200 mg, 416.97 μmol, 1 eq) and (4-nitrophenyl)methanethiol (282.21 mg, 1.67 mmol, 4 eq) in EtOH (10 mL) was added TEA (210.96 mg, 2.08 mmol, 290.18 μL, 5 eq). The mixture was stirred at 80° C. for 2 hours. The mixture was then concentrated under vacuum. The resultant residue was purified by Prep-TLC (Petroleum ether/Ethyl acetate=2/1, Rf=0.3) to afford the corresponding thioether (116 mg, 45% yield) as a yellow oil.

LCMS: Rt=1,050 min; (ESI positive ion) m/z: 612.9 (M+H)+(calculated: 613.02).

To a mixture of the above thioether compound (116 mg, 189.42 μmol, 1 eq), 2,4,6-trimethyl-1,3,5,2,4,6-trioxatriborinane (237.79 mg, 947.10 μmol, 264.80 μL, 50% purity, 5 eq) and K2CO3 (65.45 mg, 473.55 μmol, 2.5 eq) in dioxane (5 mL) and water (0.5 mL) was added Pd(dppf)Cl2 (13.86 mg, 18.94 μmol, 0.1 eq). The mixture was stirred at 80° C. for 16 hr under N2 atmosphere. The reaction mixture was concentrated to give a residue, which was purified by Prep-TLC (Petroleum ether/Ethyl acetate=1/1, RF=0.4) to give the crude product. The crude product was purified by Prep-HPLC (column: Phenomenex luna C18 150×25 mm×10 μm; mobile phase: [water(FA)-ACN]; B %: 39%-69%, 10 min). The purified solution was lyophilized to give a mixture of diastereomers, which was separated by SFC (column: DAICEL CHIRALCEL OD(250 mm×30 mm, 10 um); mobile phase: [0.10% NH3·H2O MEOH]; B %: 35%; 55 min) to afford Compound 194 (11.25 mg, 28% yield) as a white solid and Compound 286 (2.8 mg, 7% yield) as a white solid.

### Compound 194:

LCMS: Rt=0.944 min; (ESI positive ion) m/z: 417.0 (M+H)+(calculated: 417.12).

1H NMR (400 MHz, DMSO-d6) δ=8.59 (s, 1H), 8.19-8.14 (m, 2H), 7.74 (d, J=8.8 Hz, 2H), 7.43 (d, J=0.8 Hz, 1H), 6.07 (d, J=5.1 Hz, 1H), 5.34 (d, J=5.9 Hz, 1H), 5.14 (d, J=5.4 Hz, 1H), 4.76 (d, J=1.8 Hz, 2H), 4.35 (q, J=5.4 Hz, 1H), 3.95-3.88 (m, 1H), 3.85 (q, J=5.0 Hz, 1H), 2.40 (d, J=0.8 Hz, 3H), 1.27 (d, J=6.4 Hz, 3H).

### Compound 286:

LCMS: Rt=0.922 min; (ESI positive ion) m/z: 417.0 (M+H)+(calculated: 417.12).

1H NMR (400 MHz, DMSO-d6) δ=8.56 (s, 1H), 8.19-8.15 (m, 2H), 7.74 (d, J=8.8 Hz, 2H), 7.45 (d, J=1.0 Hz, 1H), 6.53 (d, J=4.9 Hz, 1H), 5.25 (d, J=5.1 Hz, 1H), 5.19 (d, J=6.3 Hz, 1H), 4.75 (d, J=3.5 Hz, 2H), 4.23 (q, J=4.9 Hz, 1H), 4.11 (quin, J=6.3 Hz, 1H), 3.86-3.79 (m, 1H), 2.38 (d, J=0.8 Hz, 3H), 1.22 (d, J=6.3 Hz, 3H).

### Compound 197:

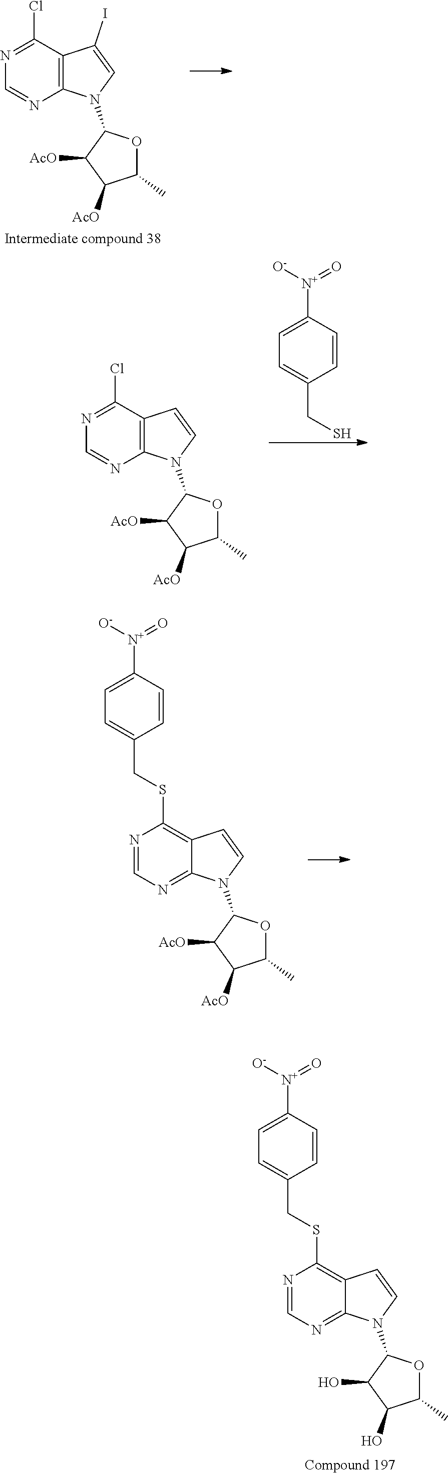

To a solution of Intermediate Compound 38 (100.00 mg, 208.48 μmol, 1 eq) in THE (2 mL) was added isopropylmagnesium chloride-lithium chloride (1.3 M, 320.74 μL, 2 eq) at −10′° C. The mixture was stirred at −10° C. for 1 hr under N2 atmosphere. The mixture was then poured into an ice cold aqueous solution of 1N HCl (20 mL). The mixture was extracted with EtOAc (10 mL×2) and the combined organic phase was washed with brine (20 mL), dried with anhydrous Na2SO4, filtered and concentrated under vacuum. The resultant residue was purified by Prep-TLC (Petroleum ether/Ethyl acetate=3/1) to afford the corresponding deiodized compound (48 mg, 59% yield) as a yellow oil.

LCMS: Rt=0.877 min; (ESI positive ion) m/z: 353.8 (M+H)+(calculated: 354.08).

1H NMR (400 MHz, CDCl₃) δ=8.68 (s, 1H), 7.38 (d, J=3.8 Hz, 1H), 6.71 (d, J=3.8 Hz, 1H), 6.38 (d, J=5.6 Hz, 1H), 5.75 (t, J=5.6 Hz, 1H), 5.30 (t, J=5.4 Hz, 1H), 4.33-4.29 (m, 1H), 2.16 (s, 3H), 2.08-2.05 (m, 3H), 1.50 (d, J=6.4 Hz, 3H).

To a mixture of the above deiodized compound (66 mg, 186.57 μmol, 1 eq) and (4-nitrophenyl)methanethiol (126.27 mg, 746.27 μmol, 4 eq) in EtOH (1 mL) was added TEA (94.39 mg, 932.84 μmol, 129.84 μL, 5 eq). The mixture was stirred at 80° C. for 2 hours and then concentrated under vacuum. The resultant residue was purified by Prep-TLC (Petroleum ether/Ethyl acetate=3/1) to afford the corresponding thioether compound (58 mg, 64% yield) as a yellow oil.

LCMS: Rt=0.733 min; (ESI positive ion) m/z: 487.0 (M+H)+(calculated: 487.12).

To a solution of the above thioether compound (58.00 mg, 119.22 μmol, 1 eq) in MeOH (1 mL) was added NH3·H2O (910.00 mg, 7.27 mmol, 1 mL, 28% purity, 60.98 eq). The mixture was stirred at 25° C. for 0.5 hr and then concentrated under vacuum. The resultant residue was purified by Prep-HPLC (column: Phenomenex C18 75×30 mm×3 μm; mobile phase: [water(FA)-ACN]; B %: 35%-65%, 7 min) to afford Compound 197 (4.89 mg, 10% yield) as a white solid.

LCMS: Rt=0.884 min; (ESI positive ion) m/z: 403.1 (M+H)+(calculated: 403.10).

1H NMR (400 MHz, DMSO-d6) δ=8.68 (s, 1H), 8.16 (d, J=8.6 Hz, 2H), 7.74 (d, J=8.6 Hz, 2H), 7.71 (d, J=3.8 Hz, 1H), 6.61 (d, J=3.8 Hz, 1H), 6.10 (d, J=5.0 Hz, 1H), 5.39 (br d, J=5.6 Hz, 1H), 5.17 (br d, J=5.4 Hz, 1H), 4.78 (s, 2H), 4.45 (q, J=5.6 Hz, 1H), 3.98-3.88 (m, 2H), 1.28 (d, J=6.4 Hz, 3H).

### Compound 199:

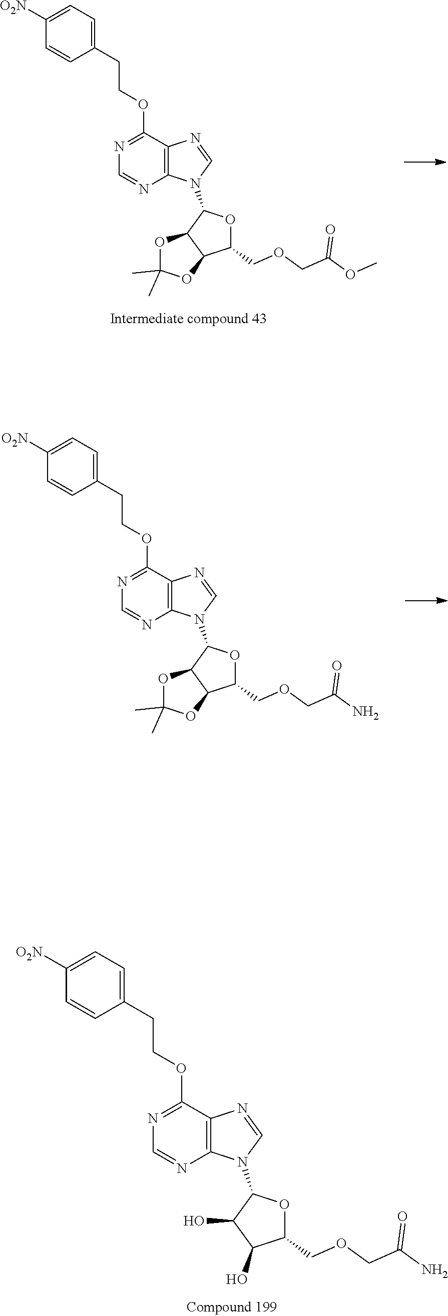 [SMILES: COC(=O)COC[C@H]1O[C@@H](n2cnc3c(OCCc4ccc([N+](=O)[O-])cc4)ncnc32)[C@@H]2OC(C)(C)O[C@@H]21 ; CC1(C)O[C@@H]2[C@H](O1)[C@@H](COCC(N)=O)O[C@H]2n1cnc2c(OCCc3ccc([N+](=O)[O-])cc3)ncnc21 ; NC(=O)COC[C@H]1O[C@@H](n2cnc3c(OCCc4ccc([N+](=O)[O-])cc4)ncnc32)[C@H](O)[C@@H]1O]

A mixture of Intermediate Compound 43 (100 mg, 188.86 μmol, 1 eq) and NH3/MeOH (7 M, 5 mL, 185.32 eq) was stirred at 25° C. for 12 hr. The mixture was then concentrated under vacuum and the resultant residue was purified by Prep-TLC (Petroleum ether/Ethyl acetate=0/1) to afford the corresponding amide compound (65 mg, 67% yield) as a colorless oil.

LCMS: Rt=0.875 min; (ESI positive ion) m/z: 515.0 (M+H)+(calculated: 515.18).

A solution of the above amide compound (60 mg, 116.62 μmol, 1 eq) in formic acid (0.8 mL) and water (0.2 mL) was stirred at 20° C. for 1 hr. The mixture was then concentrated under vacuum and purified by Prep-HPLC (column: Unisil 3-100 C18 Ultra 150×50 mm×3 μm; mobile phase: [water(FA)-ACN]; B %: 15%-45%, 15 min) to afford Compound 199 (35.41 mg, 64% yield) as a white solid.

LCMS: Rt=0.783 min; (ESI positive ion) m/z: 474.9 (M+H)+(calculated: 475.15).

1H NMR (400 MHz, DMSO-d6) δ=8.63 (s, 1H), 8.54 (s, 1H), 8.18 (d, J=8.6 Hz, 2H), 7.64 (d, J=8.6 Hz, 2H), 7.27 (br s, 1H), 7.19 (br s, 1H), 5.98 (d, J=5.6 Hz, 1H), 5.54 (d, J=6.0 Hz, 1H), 5.34 (d, J=5.0 Hz, 1H), 4.84 (t, J=6.4 Hz, 2H), 4.67 (q, J=5.4 Hz, 1H), 4.24 (q, J=4.8 Hz, 1H), 4.08 (q, J=3.8 Hz, 1H), 3.86 (s, 2H), 3.72-3.64 (m, 2H), 3.31-3.29 (m, 2H).

### Compound 203:

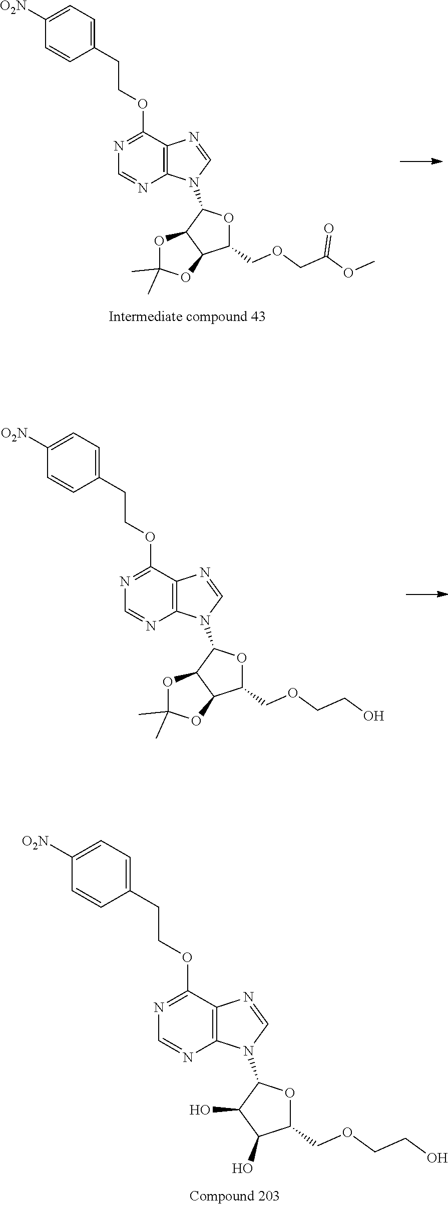 [SMILES: COC(=O)COC[C@H]1O[C@@H](n2cnc3c(OCCc4ccc([N+](=O)[O-])cc4)ncnc32)[C@@H]2OC(C)(C)O[C@@H]21 ; CC1(C)O[C@@H]2[C@H](O1)[C@@H](COCCO)O[C@H]2n1cnc2c(OCCc3ccc([N+](=O)[O-])cc3)ncnc21 ; O=[N+]([O-])c1ccc(CCOc2ncnc3c2ncn3[C@@H]2O[C@H](COCCO)[C@@H](O)[C@H]2O)cc1]

To a solution of Intermediate Compound 43 (60 mg, 113.31 μmol, 1 eq) in THE (1 mL) was added LiBH4 (2 M, 84.99 μL, 1.5 eq) at 0° C. The mixture was stirred at 0° C. for 1 hr. The mixture was then added to ice-water (10 mL) dropwise at 0° C. and stirred for 5 min. The mixture was extracted with EtOAc (5 mL×2) and the combined organic phase was washed with brine (10 mL), dried with anhydrous Na2SO4, filtered and concentrated under vacuum. The resultant residue was purified by Prep-TLC (Petroleum ether/Ethyl acetate=0/1) to afford the corresponding alcohol compound (32 mg, 56% yield) as a yellow oil.

LCMS: Rt=0.900 min; (ESI positive ion) m/z: 502.2 (M+H)+(calculated: 502.19).

A solution of the above alcohol compound (32 mg, 63.81 μmol, 1 eq) in formic acid (0.8 mL) and water (0.2 mL) was stirred at 20° C. for 1 hr. The mixture was concentrated under vacuum and the resultant residue was purified by Prep-TLC (DCM/MeOH=10/1) to afford Compound 203 (7.94 mg, 27% yield) as a white solid.

LCMS: Rt=0.806 min; (ESI positive ion) m/z: 461.9 (M+H)+(calculated: 462.15).

1H NMR (400 MHz, DMSO-d6) δ=8.63 (s, 1H), 8.54 (s, 1H), 8.18 (d, J=8.6 Hz, 2H), 7.64 (d, J=8.8 Hz, 2H), 6.00 (d, J=5.6 Hz, 1H), 5.52 (d, J=6.0 Hz, 1H), 5.31 (d, J=5.0 Hz, 1H), 4.84 (t, J=6.4 Hz, 2H), 4.69 (t, J=5.2 Hz, 1H), 4.59 (q, J=5.6 Hz, 1H), 4.20-4.15 (m, 1H), 4.05 (q, J=3.8 Hz, 1H), 3.68-3.64 (m, 1H), 3.62-3.58 (m, 1H), 3.54-3.51 (m, 2H), 3.48-3.45 (m, 2H), 3.31 (br s, 2H).

### Compound 208:

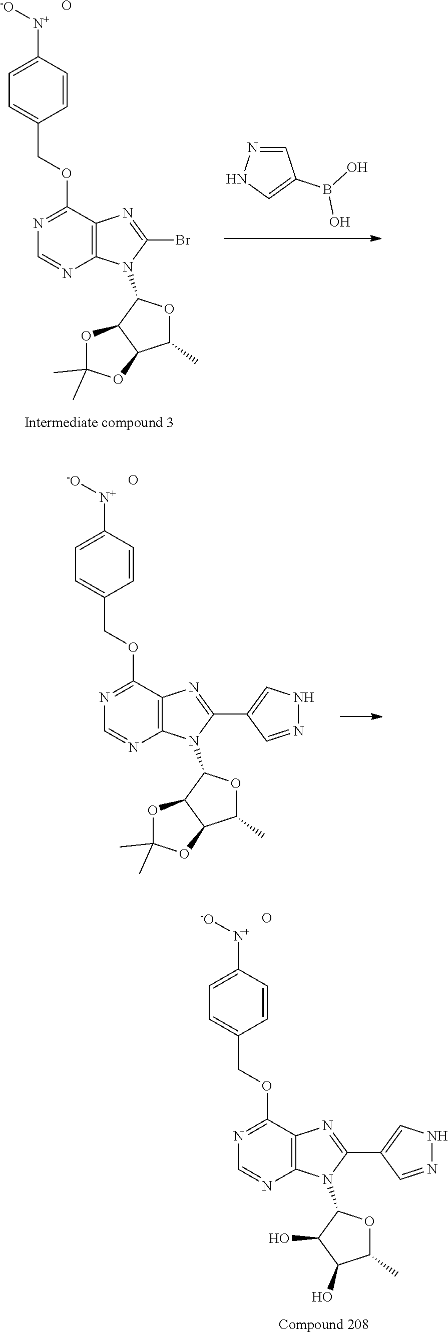

To a solution of Intermediate Compound 3 (230 mg, 454.27 μmol, 1 eq) in dioxane (4 mL) was added Cs2CO3 (888.06 mg, 2.73 mmol, 6 eq), (1H-pyrazol-4-yl)boronic acid (203.32 mg, 1.82 mmol, 4 eq), Pd(dppf)Cl2·CH2Cl2 (74.20 mg, 90.85 μmol, 0.2 eq) and water (0.4 mL). The mixture was stirred at 100° C. for 12 hr under N2. The reaction mixture then was filtered and the filter cake was washed with EtOAc (10 mL×2). The filtrate was concentrated to give a residue, which was purified by Prep-TLC (SiO2, PE:EtOAc=0:1) to afford the corresponding pyrazole compound (136 mg, 61% yield) as a red oil.

LCMS: Rt=0.565 min; (ESI positive ion) m/z: 494.0 (M+H)+(calculated: 494.17).

A solution of above functionalized compound (145 mg, 293.84 μmol, 1 eq) in HCOOH/H2O (4/1 V/V) (3 mL) was stirred at 0° C. for 10 hr. The reaction mixture was adjusted to a pH of 7-8 with NH3·H2O at 0° C. Then H2O (10 mL) was added to the mixture and the mixture was extracted with EtOAc (20 mL×3). The combined organic layer layers were washed with brine, dried by Na2SO4 and concentrated to give a residue. The residue was purified by Prep-HPLC (column: Waters xbridge 150×25 mm 10 μm; mobile phase: [water (NH4HCO3)-ACN]; B %: 19%-49%, 8 min). The purified solution was lyophilized to afford Compound 208 (9.87 mg, 7% yield) as a white solid.

LCMS: Rt=0.549 min; (ESI positive ion) m/z: 454.2 (M+H)+(calculated: 454.14).

1H NMR (400 MHz, DMSO-d6) δ=13.54 (br d, J=2.0 Hz, 1H), 8.53 (s, 1H), 8.42-8.21 (m, 3H), 8.10-7.94 (m, 1H), 7.77 (d, J=8.8 Hz, 2H), 5.85 (d, J=4.9 Hz, 1H), 5.80 (s, 2H), 5.42-5.38 (m, 1H), 5.35 (q, J=5.4 Hz, 1H), 5.20 (d, J=5.6 Hz, 1H), 4.13 (q, J=5.3 Hz, 1H), 4.06-3.98 (m, 1H), 1.32 (d, J=6.4 Hz, 3H).

### Compound 209:

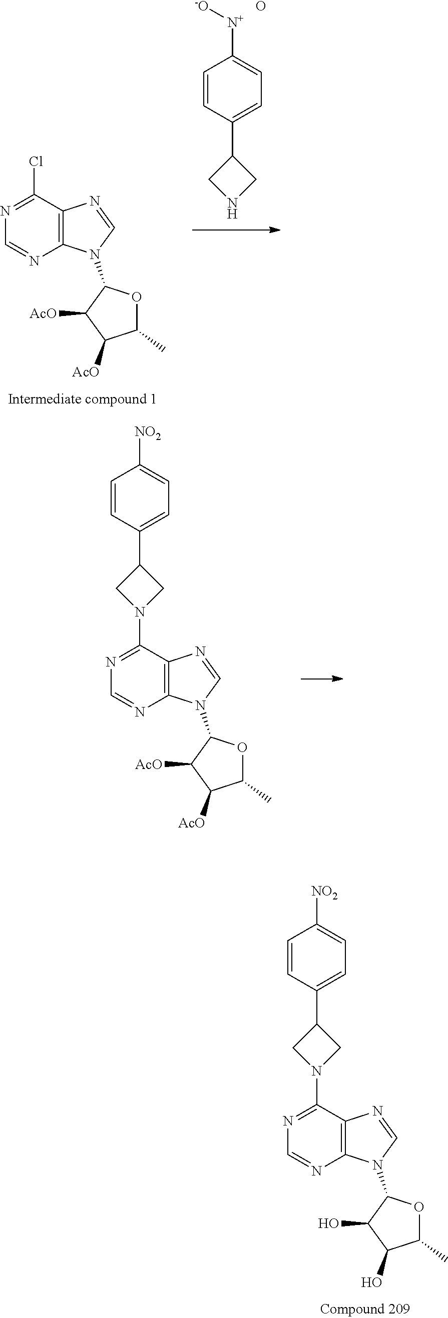

Compound 209 (48 mg, 42% yield) was synthesized from Intermediate Compound 1 and the 3-(4-nitrophenyl)azetidine following the protocols described for Compound 177.

LCMS: Rt=0.779 min; (ESI positive ion) m/z: 412.9 (M+H)+(calculated: 413.15).

1H NMR (400 MHz, DMSO-d6) δ=8.36 (s, 1H), 8.28 (s, 1H), 8.25-8.20 (m, 2H), 7.74 (d, J=8.8 Hz, 2H), 5.87 (d, J=4.8 Hz, 1H), 5.45 (br d, J=5.8 Hz, 1H), 5.18 (br d, J=4.6 Hz, 1H), 4.99-4.71 (m, 2H), 4.65 (q, J=4.8 Hz, 1H), 4.44-4.24 (m, 3H), 4.00-3.95 (m, 2H), 1.30 (d, J=6.0 Hz, 3H).

### Compound 210:

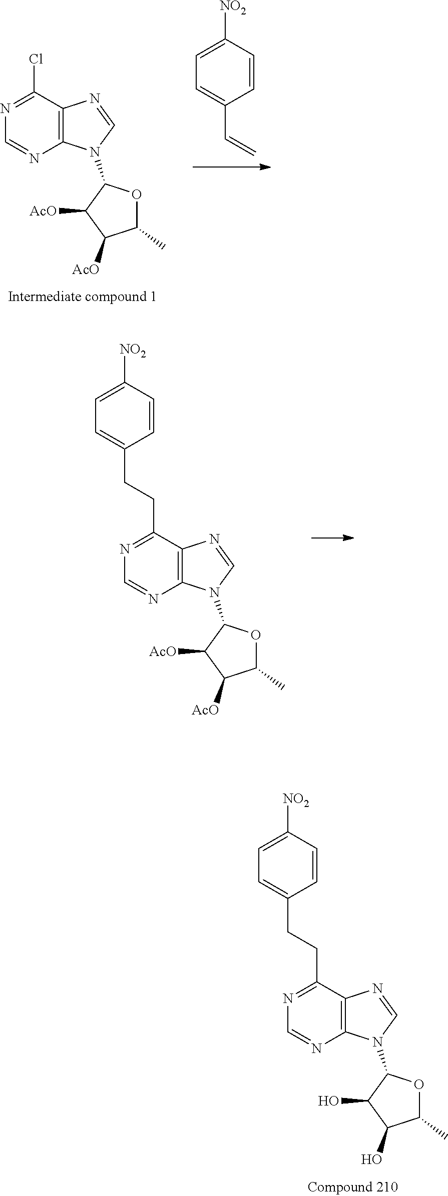 [SMILES: CC(=O)O[C@@H]1[C@H](OC(C)=O)[C@@H](C)O[C@H]1n1cnc2c(Cl)ncnc21 ; C=Cc1ccc([N+](=O)[O-])cc1 ; CC(=O)O[C@@H]1[C@H](OC(C)=O)[C@@H](C)O[C@H]1n1cnc2c(CCc3ccc([N+](=O)[O-])cc3)ncnc21 ; C[C@H]1O[C@@H](n2cnc3c(CCc4ccc([N+](=O)[O-])cc4)ncnc32)[C@H](O)[C@@H]1O]

To a solution of 1-nitro-4-vinylbenzene (100 mg, 670.48 μmol, 1 eq) in THF (5 mL) was added 9-(9-borabicyclo[3.3.1]nonan-9-yl)-9-borabicyclo[3.3.1]nonane (324.53 mg, 1.34 mmol, 2 eq). The mixture was stirred at 60° C. for 1 hr. A solution of 9-[2-(4-nitrophenyl)ethyl]-9-borabicyclo[3.3.1]nonane (0.134 M) in THF (5 mL) was obtained, which was used directly in the next step.

A mixture of Intermediate Compound 1 (150 mg, 422.84 μmol, 1 eq), 9-[2-(4-nitrophenyl)ethyl]-9-borabicyclo[3.3.1]nonane (0.134 M, 4.73 mL, 1.5 eq) from the previous step, Pd(dppf)Cl2 (30.94 mg, 42.28 μmol, 0.1 eq), and K3PO4 (179.51 mg, 845.68 μmol, 2 eq) in THF (5 mL) was degassed and purged with N2 3 times. The mixture was stirred at 60° C. for 1 hr under N2 atmosphere. The mixture was filtered and concentrated under vacuum. The resultant residue was purified by column chromatography (SiO2, DCM/MeOH=10/1) to afford the coupled compound (180 mg) as a yellow solid, which was used directly in the next step.

LCMS: Rt=0.883 min; (ESI positive ion) m/z: 469.9 (M+H)+(calculated: 470.16).

To a solution of the above coupled compound (180 mg, 383.43 μmol, 1 eq) in MeOH (2 mL) was added NH3·H2O (1.93 g, 15.40 mmol, 2.12 mL, 28% purity, 40.15 eq). The mixture was stirred at 50° C. for 1 hr. The reaction mixture was then adjusted to pH˜7 with a solution of 1N HCL. Then the mixture was filtered and concentrated under vacuum. The resultant residue was purified by Prep-HPLC (column: Phenomenex luna C18 150×25 mm×10 μm; mobile phase: [water(0.225% FA)-ACN]; B %: 20%-50%, 10 min) to afford Compound 210 (15.6 mg, 10% yield) as an off-white solid.

LCMS: Rt=0.790 min; (ESI positive ion) m/z: 385.9 (M+H)+(calculated: 386.14).

1H NMR (400 MHz, DMSO-d6) δ=8.89 (s, 1H), 8.76 (s, 1H), 8.12 (d, J=8.6 Hz, 2H), 7.54 (d, J=8.6 Hz, 2H), 5.97 (d, J=4.8 Hz, 1H), 4.72 (t, J=4.6 Hz, 1H), 4.01 (br d, J=4.8 Hz, 2H), 3.49 (br d, J=8.0 Hz, 2H), 3.38-3.33 (m, 2H), 1.32 (br d, J=5.8 Hz, 3H).

### Compound 214:

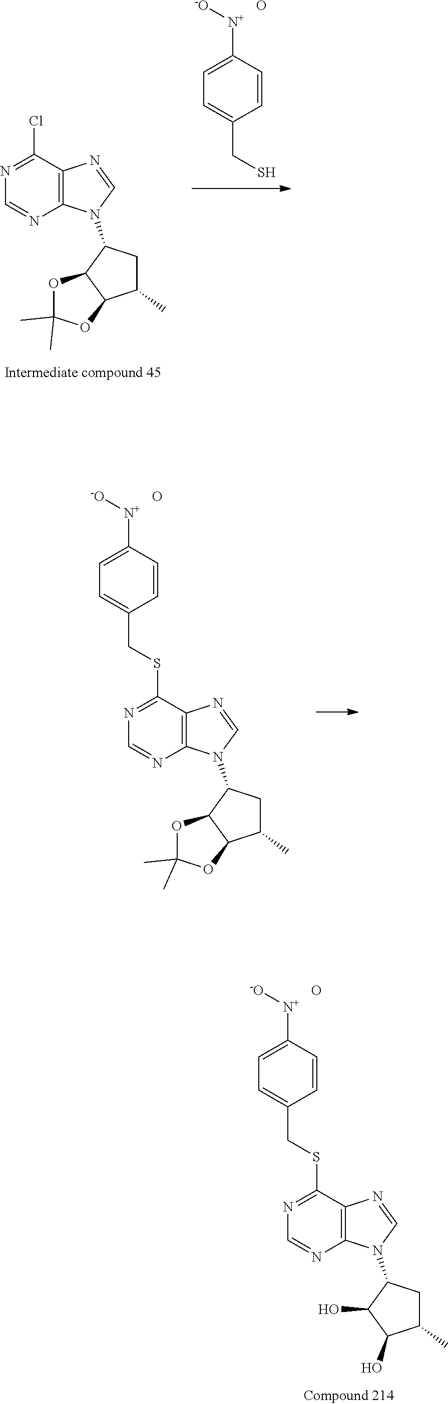

To a solution of Intermediate Compound 45 (300 mg, 971.62 μmol, 1 eq) and (4-nitrophenyl)methanethiol (657.60 mg, 3.89 mmol, 4 eq) in EtOH (5 mL) was added TEA (491.59 mg, 4.86 mmol, 676.18 μL, 5 eq). The mixture was stirred at 80° C. for 2 hr. The reaction mixture was then concentrated under reduced pressure to remove the solvent. The resultant residue was purified by column chromatography (SiO2, Petroleum ether/Ethyl acetate=100/0 to 2/1, TLC (SiO2, Petroleum ether/Ethyl acetate=3:1, Rf=0.3)) to afford the corresponding thioether (160 mg, 37% yield) as a yellow oil.

LCMS: Rt=0.949 min; (ESI positive ion) m/z: 442.1 (M+H)+(calculated: 442.15).

1H NMR (400 MHz, CDCl₃) δ=8.72 (s, 1H), 8.15 (d, J=8.7 Hz, 2H), 8.01 (s, 1H), 7.66 (d, J=8.6 Hz, 2H), 5.07 (dd, J=5.6, 7.3 Hz, 1H), 4.81-4.75 (m, 1H), 4.72 (s, 2H), 4.45 (dd, J=5.5, 7.1 Hz, 1H), 2.50-2.43 (m, 1H), 2.31-2.23 (m, 2H), 1.57 (s, 3H), 1.31 (s, 3H), 1.25 (d, J=6.4 Hz, 3H).

To a solution of the above thioether (80 mg, 181.20 μmol, 1 eq) in water (0.25 mL) was added formic acid (1.22 g, 26.51 mmol, 1 mL) at 0° C. The mixture was stirred at 20° C. for 0.5 hr. NH3·H2O (5 mL) was added to the reaction mixture at 0° C. until a pH of about 7 was reached. The mixture was then filtered and concentrated under reduced pressure to give a residue. The residue was purified by Prep-HPLC (column: Phenomenex C18 75×30 mm×3 μm; mobile phase: [water (FA)-ACN]; B %: 30%-60%, 7 min). The purified solution was lyophilized to afford Compound 214 (38.2 mg, 52% yield) as a yellow solid.

LCMS: Rt=0.767 min; (ESI positive ion) m/z: 402.2 (M+H)+(calculated: 402.12).

1H NMR (400 MHz, DMSO-d6) δ=8.74 (s, 1H), 8.58 (s, 1H), 8.16 (d, J=8.3 Hz, 2H), 7.74 (d, J=8.3 Hz, 2H), 5.09-4.88 (m, 1H), 4.84-4.71 (m, 4H), 4.42-4.33 (m, 1H), 3.65 (br t, J=4.8 Hz, 1H), 2.30 (td, J=8.1, 12.6 Hz, 1H), 2.02-1.90 (m, 1H), 1.73-1.60 (m, 1H), 1.12 (d, J=7.0 Hz, 3H).

### Compound 215:

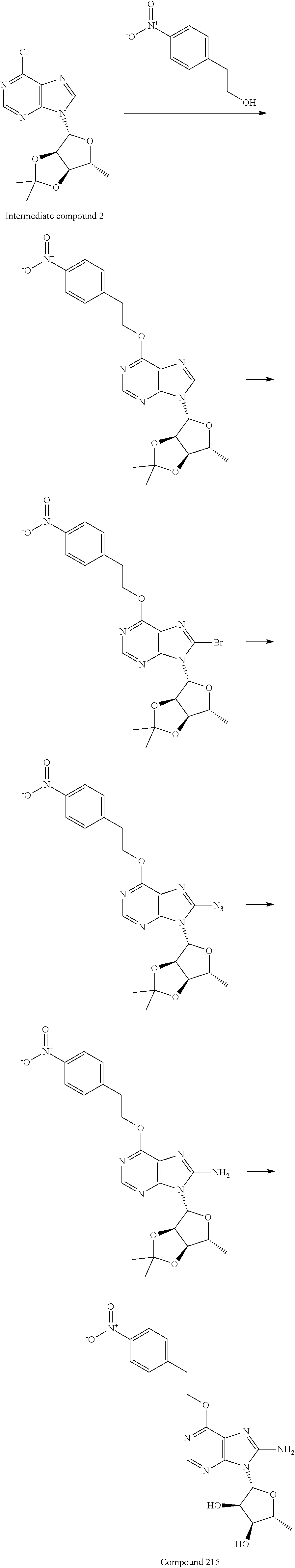

To a solution of Intermediate Compound 2 (500 mg, 1.61 mmol, 1 eq) and 2-(4-nitrophenyl)ethan-1-ol (349.87 mg, 2.09 mmol, 1.3 eq) in toluene (10 mL) was added Cs2CO3 (1.31 g, 4.03 mmol, 2.5 eq), Xantphos (186.31 mg, 322.00 μmol, 0.2 eq) and Pd2(dba)3 (147.43 mg, 161.00 μmol, 0.1 eq). The mixture was stirred at 80° C. for 0.5 hr. The reaction mixture was then filtered through a pad of silica gel. The filtrate was evaporated under reduced pressure to give a residue, which was purified by Prep-HPLC (column: Waters Xbridge C18 150×50 mm×10 μm; mobile phase: [water (NH4HCO3)-ACN]; B %: 39%-69%, 11 min). The purified solution was lyophilized to afford the corresponding ether compound (370 mg, 52% yield) as a white solid. LCMS: Rt=0.965 min; (ESI positive ion) m/z: 442.2 (M+H)+(calculated: 442.16).

To a solution of the above ether compound (120 mg, 271.84 μmol, 1 eq) in THE (2 mL) was added lithium diisopropylamide (2 M, 679.60 μL, 5 eq) dropwise at −70° C. The mixture was stirred for 1.5 hr. After 1.5 hr, a solution of carbon tetrabromide (360.60 mg, 1.09 mmol, 4 eq) in THE (2 mL) was dropwise added to the reaction mixture. The mixture was stirred for 1 hr at −70° C. The reaction mixture was poured into a saturated aqueous solution of NH4Cl (40 mL) slowly and stirred at 0° C. for 10 min. T the mixture was then extracted with EtOAc (30 mL×3). The organic layer was washed with brine, dried by Na2SO4, and concentrated to give a residue. The residue was purified by Prep-TLC (SiO2, Petroleum ether/Ethyl acetate=2/1, Rf=0.6) to afford the corresponding brominated compound (30 mg, 57.66 μmol, 21.21% yield) as a white solid.

LCMS: Rt=1.029 min; (ESI positive ion) m/z: 519.9 (M+H)+(calculated: 520.08).

To a solution of the above brominated compound (35 mg, 67.26 μmol, 1 eq) in DMF (3 mL) was added sodium azide (17.49 mg, 269.06 μmol, 4 eq). The mixture was stirred at 70° C. for 5 hr. The reaction mixture was then poured into water (20 mL) and the pH was adjusted to >9 with a saturated aqueous solution of Na2CO3 at 0° C. Then the mixture was extracted with EtOAc (10 mL×3). The organic layer was washed with brine, dried by Na2SO4, and concentrated to afford the corresponding azide compound (30 mg, crude) as a yellow oil.

LCMS: Rt=1.040 min; (ESI positive ion) m/z: 483.0 (M+H)+(calculated: 483.17).

To a solution of the above azide compound (30 mg, 62.18 μmol, 1 eq) in EtOAc (6 mL) was added Pd/C (6 mg, 62.18 μmol, 10% purity, 1.00 eq) under N2 atmosphere. The suspension was degassed and purged with H2 3 times. The mixture was stirred under H2 (15 Psi) at 25° C. for 2 hr. The reaction mixture was filtered and the filtrate was concentrated under vacuum to afford the amine compound (30 mg, crude) as a yellow oil.

LCMS: Rt=0.876 min; (ESI positive ion) m/z: 457.0 (M+H)+(calculated: 457.18).

To a solution of above amine compound (30 mg, 65.72 μmol, 1 eq) in water (0.3 mL) was added formic acid (3.16 mg, 65.72 μmol, 1.2 mL, 1 eq) at 0° C. The mixture was stirred at 20° C. for 0.5 hr. The reaction mixture was alkalized to pH from 7 to 10 with NH3·H2O. The aqueous layer was extracted with EtOAc (100 mL×3). The organic layer was collected and concentrated to give a residue. The residue was purified by Prep-HPLC (column: Waters Xbridge 150×25 mm×5 μm; mobile phase: [water (NH4HCO3)-ACN]; B %: 22%-52%, 9 min). The purified solution was lyophilized to afford Compound 215 (4.4 mg, 15% yield) as a white solid.

LCMS: Rt=0.793 min; (ESI positive ion) m/z: 417.0 (M+H)+(calculated: 417.14).

1H NMR (400 MHz, DMSO-d6) δ=8.21-8.14 (m, 3H), 7.61 (d, J=8.6 Hz, 2H), 6.87 (s, 2H), 5.68 (d, J=4.3 Hz, 1H), 5.26 (d, J=5.3 Hz, 1H), 5.06-4.99 (m, 2H), 4.71 (t, J=6.3 Hz, 2H), 4.11 (q, J=5.7 Hz, 1H), 3.86 (quin, J=6.1 Hz, 1H), 3.23 (t, J=6.4 Hz, 2H), 1.25 (d, J=6.2 Hz, 3H).

### Compound 216:

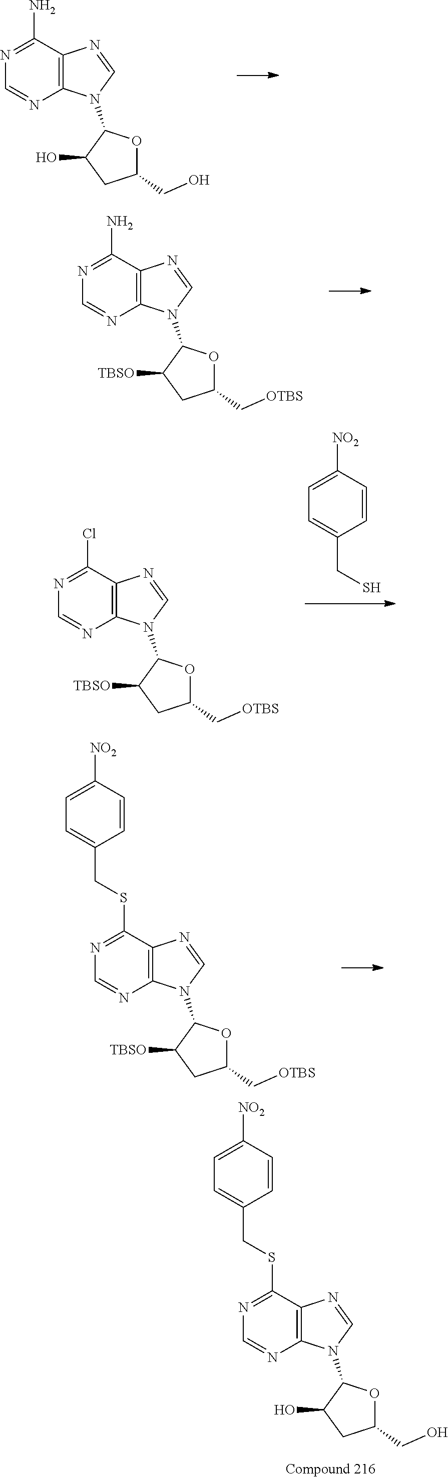

A mixture of (2R,3R,5S)-2-(6-amino-9H-purin-9-yl)-5-(hydroxymethyl)tetrahydrofuran-3-ol (1.6 g, 6.37 mmol, 1 eq) and imidazole (2.60 g, 38.21 mmol, 6 eq) in DMF (42 mL) was added a solution of tert-butyldimethylsilyl chloride (2.88 g, 19.11 mmol, 2.34 mL, 3 eq) in DMF (30 mL) dropwise under nitrogen atmosphere at 0′° C. The mixture was slowly warmed to 20° C. and stirred for 16 hr at this temperature. The reaction mixture was diluted with DCM (100 mL) and washed with a saturated aqueous solution of NH4Cl (150 mL×3), brine, and then dried by Na2SO4. The solution was concentrated to afford the corresponding protected alcohol compound (3.58 g, crude) as a white solid.

LCMS: Rt=1.023 min; (ESI positive ion) m/z: 480.2 (M+H)+(calculated: 480.27).

1H NMR (400 MHz, CDCl₃) δ=8.34 (d, J=11.0 Hz, 2H), 6.13-5.92 (m, 3H), 4.65-4.60 (m, 1H), 4.56 (dt, J=2.8, 6.2 Hz, 1H), 4.16-4.10 (m, 1H), 3.78 (dd, J=2.7, 11.6 Hz, 1H), 2.32-2.21 (m, 1H), 1.86 (ddd, J=2.1, 5.6, 12.9 Hz, 1H), 0.96-0.91 (m, 12H), 0.90 (s, 6H), 0.14-0.12 (m, 6H), 0.11-0.07 (m, 6H).

To a solution of the above protected alcohol compound (3.78 g, 7.88 mmol, 1 eq) in DCM (75 mL) was added trimethylsilyl chloride (7.70 g, 70.91 mmol, 9.00 mL, 9 eq) dropwise at 0° C., followed by a solution of tert-butyl nitrite (4.87 g, 47.27 mmol, 5.62 mL, 6 eq) in DCM (75 mL). After stirring for 30 min, the solution was warmed to 20° C. and stirred for 12 hr at this temperature. The reaction mixture was then slowly poured into a saturated aqueous solution of NaHCO3 (200 mL) under ice-water bath cooling. The aqueous phase was extracted with DCM (100 mL×2) and the combined organic phases were washed with brine, dried over anhydrous Na2SO4 and concentrated to give a residue. The residue was purified by flash silica gel chromatography (ISCO®; 40 g SepaFlash® Silica Flash Column, Eluent of 0-11% Ethyl acetate/Petroleum ether gradient @100 mL/min) to afford the corresponding chlorinated compound (1.48 g, 38% yield) as a white solid.

LCMS: Rt=1.239 min; (ESI positive ion) m/z: 499.2 (M+H)+(calculated: 499.22).

1H NMR (400 MHz, CDCl₃) δ=8.73 (d, J=7.8 Hz, 2H), 6.09 (s, 1H), 4.67-4.57 (m, 2H), 4.18 (dd, J=2.2, 11.7 Hz, 1H), 3.79 (dd, J=2.4, 11.7 Hz, 1H), 2.27 (ddd, J=4.8, 9.8, 13.1 Hz, 1H), 1.88 (ddd, J=2.1, 5.5, 13.0 Hz, 1H), 0.95 (s, 9H), 0.93-0.91 (m, 9H), 0.15 (d, J=5.4 Hz, 9H), 0.11 (s, 3H).

To a solution of the above chlorinated compound (150 mg, 300.49 μmol, 1 eq) in EtOH (3 mL) was added (4-nitrophenyl)methanethiol (76.26 mg, 450.73 μmol, 1.5 eq) and Et3N (91.22 mg, 901.46 μmol, 125.47 μL, 3 eq). The mixture was stirred at 80° C. for 4 hr. The reaction mixture was added to water (10 mL) and extracted with EtOAc (10 mL×3). The organic layer was washed with brine, dried by Na2SO4, and concentrated to afford the corresponding thioether compound (240 mg, crude) as a yellow oil.

LCMS: Rt=1.306 min; (ESI positive ion) m/z: 632.2 (M+H)+(calculated: 632.27).

To a solution of the above thioether compound (235 mg, 371.88 μmol, 1 eq) in MeOH (3 mL) was added ammonium fluoride (404 mg, 10.91 mmol, 29.33 eq). The mixture was stirred at 65° C. for 3 hr. The solvent was removed under reduced pressure to give a residue, which was purified by Prep-HPLC (column: Phenomenex C18 75×30 mm×3 μm; mobile phase: [water(FA)-can]; B %: 25%-55%, 7 min). The purified solution was lyophilized to afford Compound 216 (29.4 mg, 20% yield) as a yellow solid.

LCMS: Rt=0.848 min; (ESI positive ion) m/z: 404.1 (M+H)+(calculated: 404.10).

1H NMR (400 MHz, DMSO-d6) δ=8.75 (d, J=17.5 Hz, 2H), 8.17 (d, J=8.8 Hz, 2H), 7.75 (d, J=8.8 Hz, 2H), 5.98 (d, J=1.7 Hz, 1H), 5.72 (d, J=4.0 Hz, 1H), 5.05 (t, J=5.4 Hz, 1H), 4.79 (d, J=2.7 Hz, 2H), 4.60 (br s, 1H), 4.45-4.34 (m, 1H), 3.77-3.67 (m, 1H), 3.53 (td, J=4.6, 11.9 Hz, 1H), 2.28-2.19 (m, 1H), 1.90 (ddd, J=2.4, 6.0, 13.1 Hz, 1H).

### Compound 218:

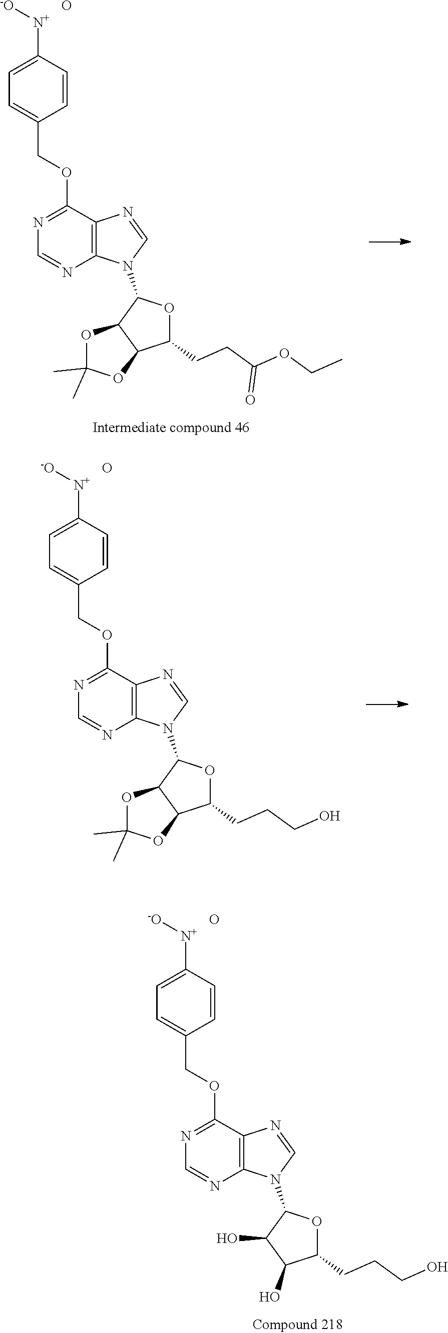

To a solution of Intermediate Compound 46 (70 mg, 136.32 μmol, 1 eq) in THE (4 mL) was added LiBH4 (2 M, 149.95 μL, 2.2 eq) at 0° C. under N2 atmosphere. The mixture was stirred at 25° C. for 16 hr. The reaction mixture was then quenched by addition of water (10 mL) at 0° C. and extracted with ethyl acetate (15 mL×2). The combined organic layers were washed with brine (20 mL), dried over Na2SO4, filtered and concentrated under reduced pressure to give the corresponding alcohol compound (65 mg, 100% yield) as a yellow oil, which was used directly in the next step.

To a solution of the above alcohol compound (45 mg, 95.45 μmol, 1 eq) in water (0.25 mL) was added formic acid (4.59 mg, 95.45 μmol, 1 mL, 1 eq) at 0° C. The mixture was stirred at 25° C. for 1 hr. The reaction mixture was then concentrated under reduced pressure at 25° C. The resultant residue was dissolved in THE (1 mL) and MeOH (1 mL). K2CO3(1.5 eq) was then added and the mixture was stirred at 25° C. for 10 min. The reaction mixture was filtered and concentrated under vacuum. The resultant residue was purified by Prep-TLC (SiO2, Petroleum ether:Ethyl acetate=0/1, the desired product Rf=0.2) and then Prep-HPLC (column: Waters Xbridge 150×25 mm×5 μm; mobile phase: [water (NH4HCO3)-ACN]; B %: 21%-51%, 9 min) to give Compound 218 (15.06 mg, 36% yield) as a yellow solid.

LCMS: Rt=0.505 min; (ESI positive ion) m/z: 432.1 (M+H)+(calculated: 432.14).

1H NMR (400 MHz, DMSO-d6) δ=8.63 (s, 1H), 8.56 (s, 1H), 8.27 (d, J=8.5 Hz, 2H), 7.76 (d, J=8.5 Hz, 2H), 5.95 (d, J=5.1 Hz, 1H), 5.80 (s, 2H), 5.48 (d, J=5.8 Hz, 1H), 5.21 (d, J=5.1 Hz, 1H), 4.69 (q, J=5.4 Hz, 1H), 4.39 (t, J=5.2 Hz, 1H), 4.05 (q, J=5.0 Hz, 1H), 3.90-3.83 (m, 1H), 3.39 (br d, J=6.4 Hz, 2H), 1.77-1.61 (m, 2H), 1.56-1.42 (m, 2H).

### Compound 219:

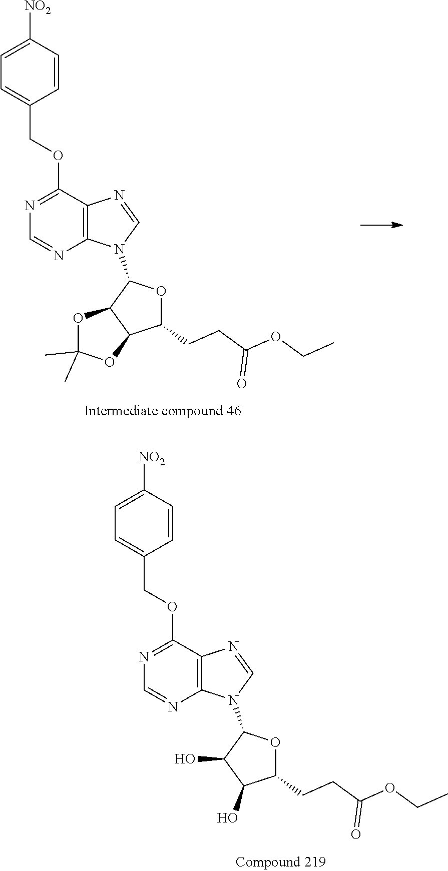 [SMILES: CCOC(=O)CC[C@H]1O[C@@H](n2cnc3c(OCc4ccc([N+](=O)[O-])cc4)ncnc32)[C@@H]2OC(C)(C)O[C@@H]21 ; CCOC(=O)CC[C@H]1O[C@@H](n2cnc3c(OCc4ccc([N+](=O)[O-])cc4)ncnc32)[C@H](O)[C@@H]1O]

To a solution of Intermediate Compound 46 (20 mg, 38.95 μmol, 1 eq) in water (0.2 mL) was added formic acid (1.87 mg, 38.95 μmol, 0.8 mL, 1 eq) at 0° C. The mixture was stirred at 20° C. for 1 hr. The reaction mixture alkalized to a pH from 7 to 10 with NH3·H2O. The aqueous layer was extracted with EtOAc (5 mL×3). The organic layer was collected and concentrated to get a residue, which was purified by Prep-HPLC (column: Phenomenex luna C18 150×25 mm×10 μm; mobile phase: [water (FA)-ACN]; B %: 26%-56%, 10 min). The purified solution was lyophilized to give Compound 219 (8.6 mg, 44% yield) as a white solid.

LCMS: Rt=0.854 min; (ESI positive ion) m/z: 474.0 (M+H)+(calculated: 474.15).

1H NMR (400 MHz, DMSO-d6) δ=8.63 (s, 1H), 8.55 (s, 1H), 8.27 (d, J=8.7 Hz, 2H), 7.76 (d, J=8.7 Hz, 2H), 5.95 (d, J=5.3 Hz, 1H), 5.80 (s, 2H), 5.55 (br d, J=4.8 Hz, 1H), 5.30 (br d, J=4.8 Hz, 1H), 4.69 (br d, J=4.8 Hz, 1H), 4.10 (br d, J=4.3 Hz, 1H), 4.00 (q, J=7.0 Hz, 2H), 3.92-3.86 (m, 1H), 2.36 (t, J=7.5 Hz, 2H), 1.99-1.90 (m, 2H), 1.12 (t, J=7.2 Hz, 3H)

### Compound 220:

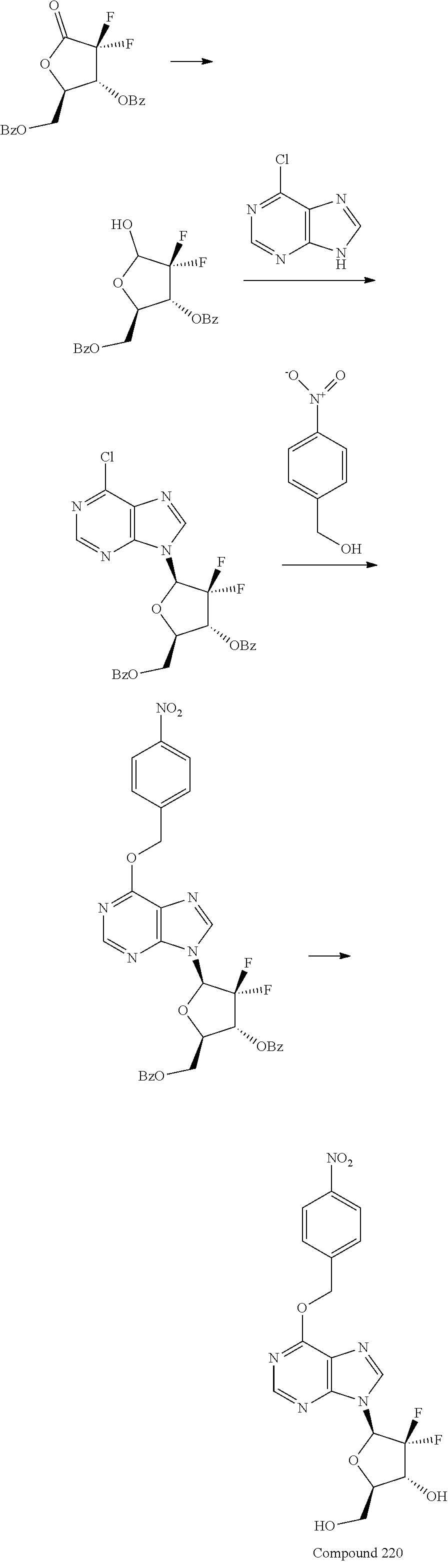

To a solution of ((2R,3R)-3-(benzoyloxy)-4,4-difluoro-5-oxotetrahydrofuran-2-yl)methyl benzoate (5 g, 13.29 mmol, 1 eq) in THE (50 mL) was added sodium bis(2-methoxyethoxy) aluminium hydride (2.95 g, 14.62 mmol, 2.84 mL, 1.1 eq). The mixture was stirred at −30° C. for 1 hr. The reaction mixture was filtered and concentrated under reduced pressure to give a residue. The residue was purified by column chromatography (SiO2, Petroleum ether/Ethyl acetate=100/1 to 1/1) to give the hemiacetal compound (4.4 g, 87% yield) as a yellow oil.

To a solution of the above hemiacetal compound (4.4 g, 11.63 mmol, 1 eq) and 6-chloro-9H-purine (1.98 g, 12.79 mmol, 1.1 eq) in dioxane (130 mL) was added triphenylphosphine (9.15 g, 34.89 mmol, 3 eq) and DEAD (6.08 g, 34.89 mmol, 6.34 mL, 3 eq). The mixture was stirred at 20° C. for 2 hr. The reaction mixture was filtered and the filter cake was washed with EtOAc (30 mL×3). The filtrate was dried by Na2SO4 and concentrated under reduced pressure to give a residue. The residue was purified by column chromatography (SiO2, Petroleum ether/Ethyl acetate=100/1 to 1/1) to give the conjugated intermediate compound (3.2 g, 52% yield) as a yellow oil.

A mixture of the above conjugated intermediate compound (500 mg, 971.13 μmol, 1 eq), (4-nitrophenyl)methanol (178.46 mg, 1.17 mmol, 1.2 eq), Cs2CO3 (791.03 mg, 2.43 mmol, 2.5 eq) and Xantphos (112.38 mg, 194.23 μmol, 0.2 eq) and Pd2(dba)3 (88.93 mg, 97.11 μmol, 0.1 eq) in dioxane (4 mL) was degassed and purged with N2 3 times. The mixture was stirred at 80° C. for 1.5 hr under N2 atmosphere. The reaction mixture was then filtered and concentrated under reduced pressure to give a residue. The residue was purified by column chromatography (SiO2, Petroleum ether/Ethyl acetate=100/1 to 1/1) to give the corresponding ether compound (400 mg, 19% yield) as a white solid.

LCMS: Rt=1.059 min; (ESI positive ion) m/z: 632.2 (M+H)+(calculated: 632.15).

To a solution of the above ether compound (400 mg, 633.37 μmol, 1 eq) in MeOH (3 mL) was added NH3·H2O (14.44 g, 115.39 mmol, 15.87 mL, 28% purity, 182.18 eq). The mixture was stirred at 20° C. for 0.5 hr. The reaction mixture was filtered and concentrated under reduced pressure to give a residue, which was purified by Prep-HPLC (column: Phenomenex luna C18 150×40 mm×15 μm; mobile phase: [water(FA)-ACN]; B %: 23%-53%, 10 min) to give Compound 220 (6.5 mg, 2% yield) as a white solid.

LCMS: Rt=0.823 min; (ESI positive ion) m/z: 424.2 (M+H)+(calculated: 424.10).

1H NMR (400 MHz, MeOD-d4) δ=8.59-8.55 (m, 2H), 8.28 (d, J=8.8 Hz, 2H), 7.81 (d, J=8.8 Hz, 2H), 6.62 (t, J=7.4 Hz, 1H), 5.84 (s, 2H), 4.54-4.49 (m, 1H), 4.09-4.04 (m, 1H), 3.90-3.82 (m, 1H), 3.79-3.74 (m, 1H)

### Compound 221:

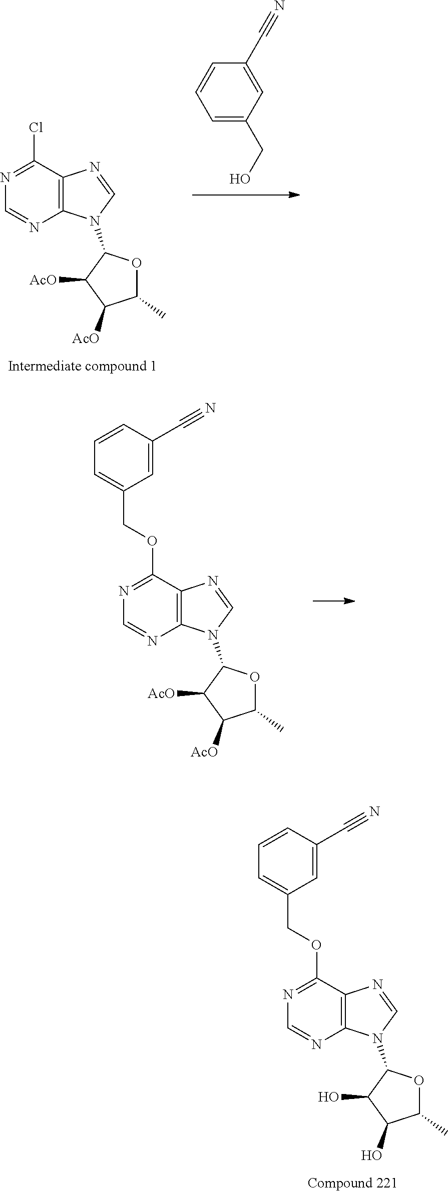 [SMILES: CC(=O)O[C@@H]1[C@H](OC(C)=O)[C@@H](C)O[C@H]1n1cnc2c(Cl)ncnc21 ; N#Cc1cccc(CO)c1 ; CC(=O)O[C@@H]1[C@H](OC(C)=O)[C@@H](C)O[C@H]1n1cnc2c(OCc3cccc(C#N)c3)ncnc21 ; C[C@H]1O[C@@H](n2cnc3c(OCc4cccc(C#N)c4)ncnc32)[C@H](O)[C@@H]1O]

A mixture of Intermediate Compound 1 (281.89 μmol, 1 eq), 3-(hydroxymethyl)benzonitrile (563.79 μmol, 2 eq), Cs2CO3 (704.73 μmol, 2.5 eq), Xantphos (56.38 μmol, 0.2 eq) and Pd2(dba)3 (28.19 μmol, 0.1 eq) in toluene (5 mL) was degassed and purged with N2 3 times. The mixture was stirred at 80° C. for 0.5 hr under N2 atmosphere. The mixture was filtered and concentrated in vacuum. The residue was purified by column chromatography (SiO2, DCM/MeOH=10/1) to afford the corresponding ether compound (crude) as yellow oil, which was used directly for the next step.

A solution of the above ether compound (crude) in MeOH/NH3·H2O (v/v=1/1) was stirred at 50° C. for 1 hr. The reaction mixture was adjusted to pH-7 by adding an aqueous solution of 1 N HCl. Then the mixture was filtered, and the filtrate was concentrated under vacuum. The residue was purified by Prep-HPLC (column: Phenomenex luna C18 150×25 mm×10 μm; mobile phase: [water(FA)-ACN]) to afford Compound 221 (20.20 mg, 19% yield over 2 steps).

LCMS: Rt=0.795 min; (ESI positive ion) m/z: 368.2 (M+H)+(calculated: 368.12).

1H NMR (400 MHz, DMSO-d6) δ=8.62 (s, 1H), 8.58 (s, 1H), 7.99 (s, 1H), 7.85 (t, J=8.2 Hz, 2H), 7.66-7.61 (m, 1H), 5.95 (d, J=5.0 Hz, 1H), 5.69 (s, 2H), 5.48 (d, J=5.6 Hz, 1H), 5.21 (d, J=5.0 Hz, 1H), 4.69 (br d, J=4.9 Hz, 1H), 4.00 (br d, J=3.9 Hz, 2H), 1.32 (br d, J=6.0 Hz, 3H)

### Compound 222:

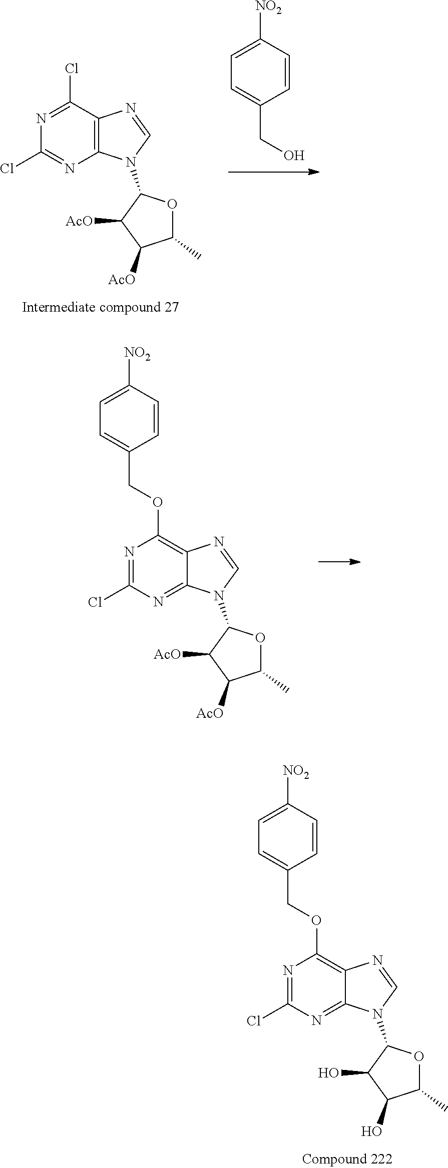 [SMILES: CC(=O)O[C@@H]1[C@H](OC(C)=O)[C@@H](C)O[C@H]1n1cnc2c(Cl)nc(Cl)nc21 ; O=[N+]([O-])c1ccc(CO)cc1 ; CC(=O)O[C@@H]1[C@H](OC(C)=O)[C@@H](C)O[C@H]1n1cnc2c(OCc3ccc([N+](=O)[O-])cc3)nc(Cl)nc21 ; C[C@H]1O[C@@H](n2cnc3c(OCc4ccc([N+](=O)[O-])cc4)nc(Cl)nc32)[C@H](O)[C@@H]1O]

Compound 222 was synthesized using the protocols described for Compound 161 from Intermediate Compound 27 and (4-nitrophenyl)methanol.

LCMS: Rt=0.891 min; (ESI positive ion) m/z: 422.0 (M+H)+(calculated: 422.08).

1H NMR (400 MHz, DMSO-d6) δ=8.66 (s, 1H), 8.31-8.26 (m, 2H), 7.79 (d, J=8.8 Hz, 2H), 5.89 (d, J=5.0 Hz, 1H), 5.77 (s, 2H), 5.53 (d, J=5.6 Hz, 1H), 5.26 (d, J=5.3 Hz, 1H), 4.62 (q, J=5.1 Hz, 1H), 4.02 (dd, J=4.6, 6.3 Hz, 1H), 3.99-3.94 (m, 1H), 1.32 (d, J=6.4 Hz, 3H)

### Compound 223:

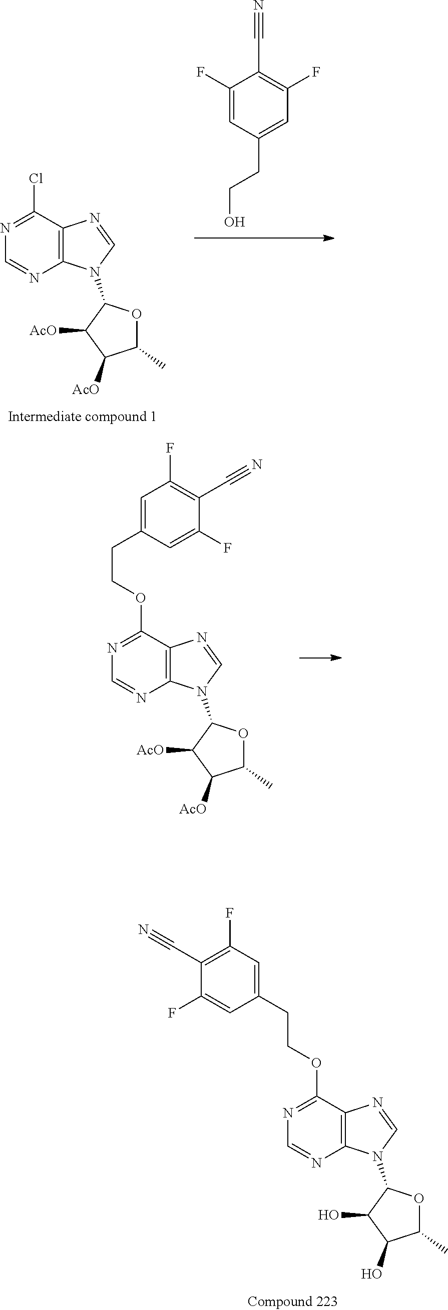 [SMILES: CC(=O)O[C@@H]1[C@H](OC(C)=O)[C@@H](C)O[C@H]1n1cnc2c(Cl)ncnc21 ; N#Cc1c(F)cc(CCO)cc1F ; CC(=O)O[C@@H]1[C@H](OC(C)=O)[C@@H](C)O[C@H]1n1cnc2c(OCCc3cc(F)c(C#N)c(F)c3)ncnc21 ; C[C@H]1O[C@@H](n2cnc3c(OCCc4cc(F)c(C#N)c(F)c4)ncnc32)[C@H](O)[C@@H]1O]

Compound 223 (20.4 mg, 27% yield) was synthesized from Intermediate Compound 1 and 2,6-difluoro-4-(2-hydroxyethyl)benzonitrile following the protocols described for Compound 188.

LCMS: Rt=0.816 min; (ESI positive ion) m/z: 417.9 (M+H)+(calculated: 418.12).

1H NMR (400 MHz, DMSO-d6) δ=8.56 (d, J=12.4 Hz, 2H), 7.50 (d, J=9.4 Hz, 2H), 5.93 (d, J=5.0 Hz, 1H), 5.47 (br d, J=5.4 Hz, 1H), 5.25-5.18 (m, 1H), 4.83 (br t, J=6.4 Hz, 2H), 4.67 (q, J=4.8 Hz, 1H), 3.99 (br d, J=4.4 Hz, 2H), 3.30-3.27 (m, 2H), 1.31 (br d, J=6.0 Hz, 3H).

### Compound 225:

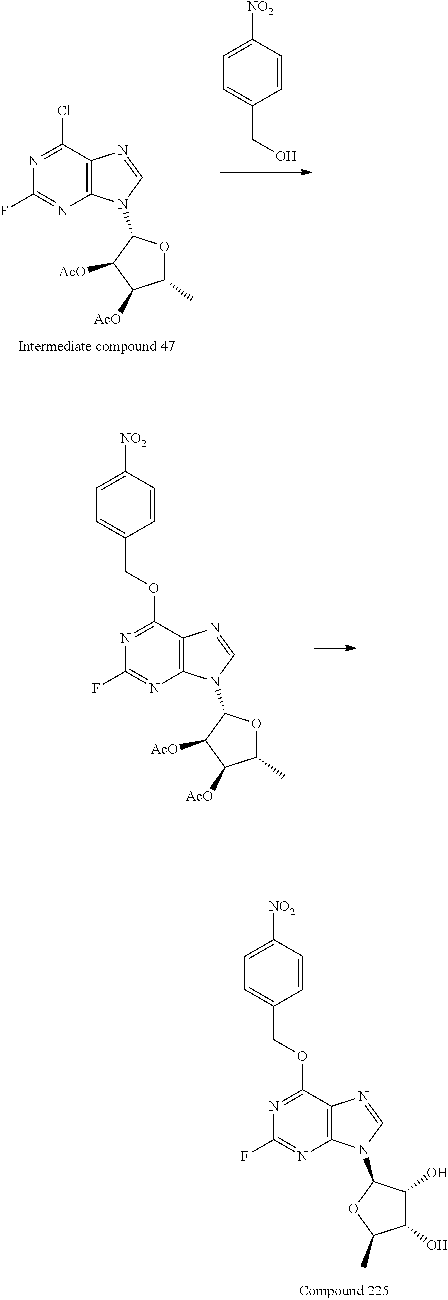 [SMILES: CC(=O)O[C@@H]1[C@H](OC(C)=O)[C@@H](C)O[C@H]1n1cnc2c(Cl)nc(F)nc21 ; O=[N+]([O-])c1ccc(CO)cc1 ; CC(=O)O[C@@H]1[C@H](OC(C)=O)[C@@H](C)O[C@H]1n1cnc2c(OCc3ccc([N+](=O)[O-])cc3)nc(F)nc21 ; C[C@H]1O[C@@H](n2cnc3c(OCc4ccc([N+](=O)[O-])cc4)nc(F)nc32)[C@H](O)[C@@H]1O]

Compound 225 (21 mg, 32% yield) was synthesized from Intermediate Compound 47 and (4-nitrophenyl)methanol following the protocols described for Compound 161.

LCMS: Rt=0.829 min; (ESI positive ion) m/z: 405.8 (M+H)+(calculated: 406.11).

1H NMR (400 MHz, DMSO-d6) δ=8.61 (s, 1H), 8.28 (d, J=8.7 Hz, 2H), 7.78 (d, J=8.7 Hz, 2H), 5.85 (d, J=5.0 Hz, 1H), 5.77 (s, 2H), 5.54 (br d, J=4.3 Hz, 1H), 5.27 (br d, J=4.6 Hz, 1H), 4.61 (br d, J=4.4 Hz, 1H), 4.05-3.93 (m, 2H), 1.31 (d, J=6.2 Hz, 3H) Compound 227:

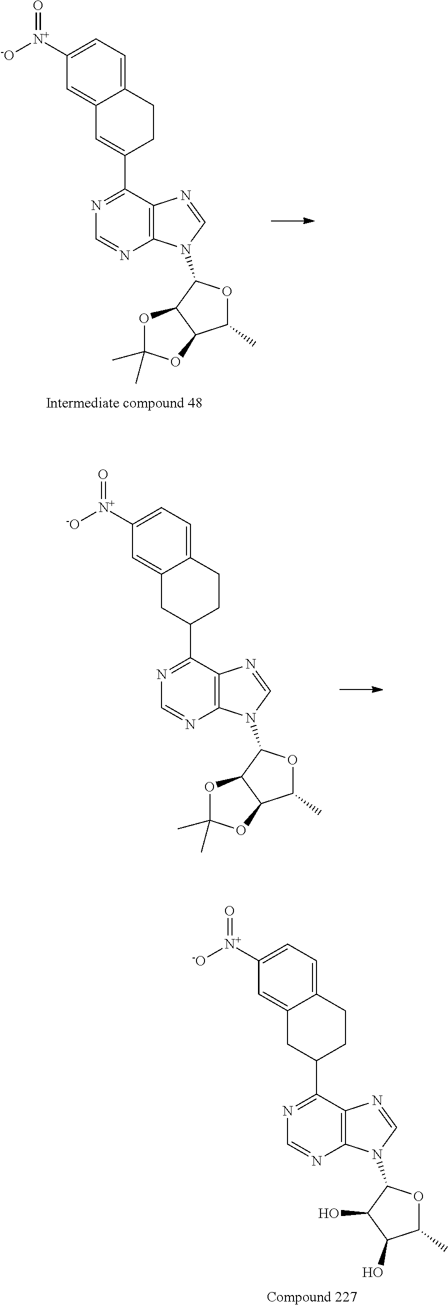

To a solution of Intermediate Compound 48 (40 mg, 89.00 μmol, 1 eq) in EtOH (8 mL) was added chlororhodium triphenylphosphane (8.23 mg, 8.90 μmol, 0.1 eg) under N2 atmosphere. The suspension was degassed and purged with H2 3 times. The mixture was stirred under H2 (50 Psi) at 80° C. for 16 hr. The reaction mixture was filtered and concentrated under reduced pressure to give a residue. The residue was purified by Prep-HPLC (column: Phenomenex C18 75×30 mm×3 um mobile phase: [water (FA)-ACN]; B %: 45%-75%, 7 min) to afford the corresponding hydrogenated compound (20 mg, 45% yield) as a brown solid.

LCMS: Rt=0.935 min; (ESI positive ion) m/z: 452.1 (M+H)+(calculated: 452.19).

1H NMR (400 MHz, DMSO-d6) δ=8.93 (s, 1H), 8.76 (s, 1H), 8.06 (s, 1H), 8.00 (dd, J=2.2, 8.4 Hz, 1H), 7.43 (d, J=8.4 Hz, 1H), 6.24 (d, J=2.4 Hz, 1H), 5.57 (td, J=2.0, 6.2 Hz, 1H), 4.85 (dd, J=3.3, 6.2 Hz, 1H), 4.35-4.26 (m, 1H), 3.86-3.77 (m, 1H), 3.40 (br s, 1H), 3.27 (br d, J=5.0 Hz, 1H), 3.04 (br d, J=5.4 Hz, 2H), 2.27-2.17 (m, 2H), 1.54 (s, 3H), 1.33 (s, 3H), 1.27 (d, J=6.6 Hz, 3H)

To a solution of the above hydrogenated compound (20 mg, 44.30 μmol, 1 eq) in water (0.1 mL) was added formic acid (488.00 mg, 8.2 m g, 0.4 mL) at 0° C. The mixture was stirred at 0° C. for 2 hr. NH3·H2O (1 mL) was added to the reaction mixture at 0° C. until the pH was 7. The mixture was then filtered and concentrated under reduced pressure to give a residue. The residue was purified by Prep-HPLC (column: Phenomenex luna C18 150×25 mm×10 μm; mobile phase: [water(FA)-ACN]; B %: 27%-57%, 10 min) to afforded Compound 227 (5.43 mg, 30% yield) as a white solid.

LCMS: Rt=0.751 min; (ESI positive ion) m/z: 412.2 (M+H)+(calculated: 412.15).

1H NMR (400 MHz, CDCl₃) δ=8.95-8.86 (m, 1H), 8.26 (s, 1H), 8.04-7.96 (m, 2H), 7.30 (d, J=8.3 Hz, 1H), 5.98 (d, J=5.3 Hz, 1H), 5.40 (br d, J=11.6 Hz, 1H), 4.70-4.64 (m, 1H), 4.48-4.40 (m, 1H), 4.22 (br s, 1H), 4.00-3.88 (m, 1H), 3.55 (ddd, J=4.5, 11.6, 16.4 Hz, 1H), 3.24 (br dd, J=4.8, 16.9 Hz, 1H), 3.10 (br d, J=5.3 Hz, 2H), 2.99 (br s, 1H), 2.39-2.27 (m, 2H), 1.44 (d, J=6.7 Hz, 3H).

### Compound 228:

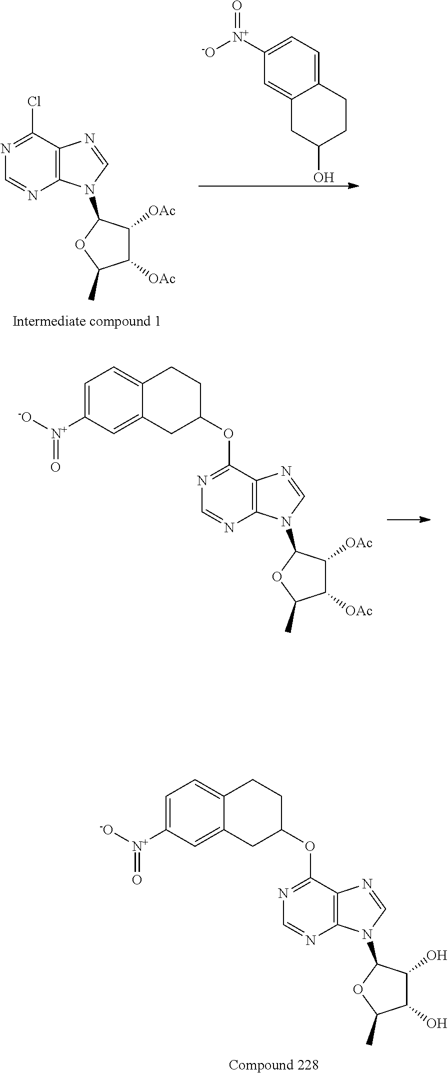

To a mixture of 7-nitro-1,2,3,4-tetrahydronaphthalen-2-ol (80 mg, 414.08 μmol, 1 eq), Intermediate Compound 1 (146.89 mg, 414.08 μmol, 1 eq) and Cs2CO3 (269.83 mg, 828.16 mol, 2 eq) in dioxane (8 mL) was added RuPhos Pd G4 (35.21 mg, 41.41 μmol, 0.1 eq) and degassed and purged with N2 3 times. The mixture was stirred at 100° C. for 16 hr under N2 atmosphere. The reaction mixture was then concentrated under vacuum and the residue was purified by column chromatography (SiO2, Petroleum ether/Ethyl acetate=1/0 to 1/2, TLC:Petroleum ether:Ethyl acetate=1/1, Rf=0.4) to afford the corresponding ether compound (80 mg, 37% yield) as a colorless oil.

1H NMR (400 MHz, CDCl₃) δ=8.56 (s, 1H), 8.04-7.96 (m, 3H), 7.31-7.27 (m, 1H), 6.11 (d, J=4.8 Hz, 1H), 6.04-5.86 (m, 2H), 5.47-5.38 (m, 1H), 4.35 (quin, J=6.0 Hz, 1H), 3.49-3.37 (m, 1H), 3.32-3.15 (m, 2H), 3.04-2.99 (m, 1H), 2.34-2.28 (m, 2H), 2.15 (s, 3H), 2.09 (s, 3H), 1.53 (d, J=6.4 Hz, 3H).

To a solution of the above ether compound (70 mg, 136.86 μmol, 1 eq) in MeOH (3.5 mL) was added NH3·H2O (3.19 g, 25.45 mmol, 3.50 mL, 28% purity, 185.94 eq). The mixture was stirred at 25° C. for 2 hr and then concentrated under reduced pressure to give a residue. The residue was purified by Prep-HPLC (column: Phenomenex luna C18 250×50 mm×15 μm; mobile phase: [water(FA)-ACN]; B %: 32%-62%, 10 min) to afford Compound 228 (40 mg, 67% yield) as a white solid.

LCMS: Rt=0.584 min; (ESI positive ion) m/z: 428.1 (M+H)+(calculated: 428.15).

1H NMR (400 MHz, DMSO-d6) δ=8.58 (s, 1H), 8.55 (s, 1H), 8.06 (s, 1H), 8.03-7.99 (m, 1H), 7.45 (d, J=8.4 Hz, 1H), 5.93 (d, J=4.8 Hz, 1H), 5.89-5.83 (m, 1H), 5.48 (br d, J=5.2 Hz, 1H), 5.21 (br d, J=4.4 Hz, 1H), 4.72-4.65 (m, 1H), 4.03-3.95 (m, 2H), 3.45-3.39 (m, 1H), 3.24-3.18 (m, 1H), 3.05-3.00 (m, 2H), 2.21 (q, J=6.4 Hz, 2H), 1.31 (d, J=6.0 Hz, 3H).

### Compound 229:

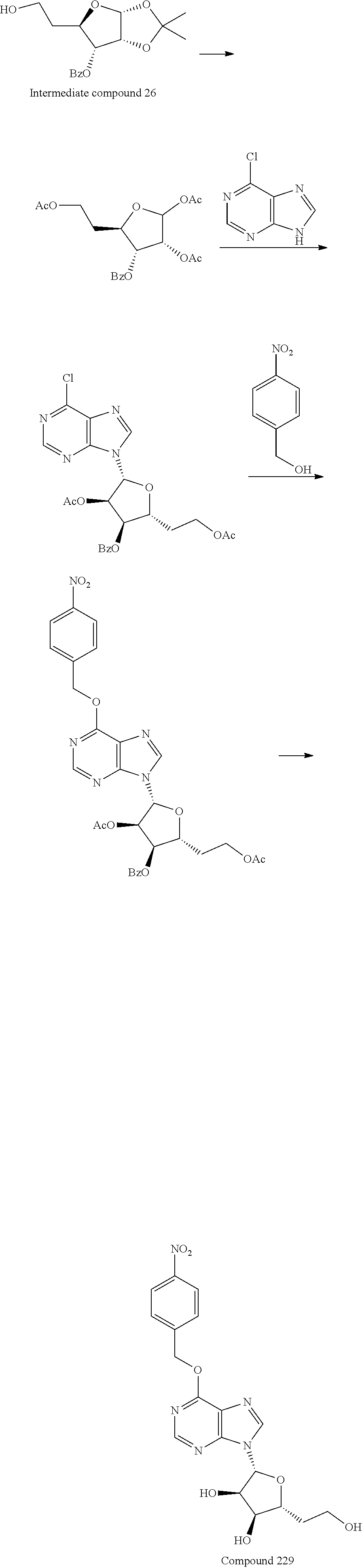

To a solution of Intermediate Compound 26 (1.2 g, 3.89 mmol, 1 eq) in AcOH (16 mL) and acetic anhydride (6 mL) was added H2SO4 (38.95 mg, 389.20 μmol, 21.17 μL, 98% purity, 0.1 eq) at 0° C. The mixture was stirred at 25° C. for 16 hr. The pH of the mixture was the adjusted to about 7 by a saturated aqueous solution of NaHCO3. The mixture was then extracted with EtOAc (50 mL×2). The combined organic phase was washed with brine (200 mL), dried with anhydrous Na2SO4, filtered and concentrated under vacuum. The residue was purified by column chromatography (SiO2, Petroleum ether/Ethyl acetate=100/1 to 3/1) to afford the acetylated compound (1.2 g, 78% yield) as a colorless oil.

A mixture of 6-chloro-9H-purine (94.06 mg, 608.56 μmol, 1.2 eq) and BSA (206.33 mg, 1.01 mmol, 250.71 μL, 2 eq) in MeCN (2 mL) was stirred at 25° C. for 1 hr. The mixture was then concentrated under vacuum to give a residue, which was redissolved with MeCN (2 mL). The above acetylated compound (200.00 mg, 507.14 μmol, 1 eq) and SnCl4 (396.36 mg, 1.52 mmol, 177.74 μL, 3 eq) were then added to the mixture at 0° C. and the mixture was stirred at 25° C. for 12 hr. The mixture was then adjusted to a pH of about 7 by a saturated aqueous solution of NaHCO3. The mixture was extracted with EtOAc (50 mL×2), dried with anhydrous Na2SO4, filtered and concentrated under vacuum. The resultant residue was purified by Prep-TLC (SiO2, Petroleum ether/Ethyl acetate=1:1) to afford the conjugated intermediate compound (55 mg, 20% yield) as a colorless oil.

LCMS: Rt=0.563 min; (ESI positive ion) m/z: 489.2 (M+H)+(calculated: 489.11).

1H NMR (400 MHz, DMSO-d6) δ=8.97 (s, 1H), 8.86 (s, 1H), 8.07-8.03 (m, 2H), 7.76-7.70 (m, 1H), 7.62-7.58 (m, 2H), 6.45 (d, J=5.6 Hz, 1H), 6.20 (t, J=5.8 Hz, 1H), 5.85-5.81 (m, 1H), 4.51-4.45 (m, 1H), 4.21-4.13 (m, 1H), 4.11-4.06 (m, 1H), 2.27-2.20 (m, 2H), 1.96 (s, 3H), 1.93 (s, 3H).

A mixture of the above conjugated intermediate compound (50.00 mg, 102.28 mol, 1 eq), (4-nitrophenyl)methanol (18.79 mg, 122.73 μmol, 1.2 eq), Cs2CO3 (83.31 mg, 255.69 μmol, 2.5 eq), Pd2(dba)3 (9.37 mg, 10.23 μmol, 0.1 eq) and Xantphos (11.84 mg, 20.46 mol, 0.2 eq) in toluene (2 mL) was degassed and purged with N2 3 times. The mixture was stirred at 80° C. for 0.5 hr under N2 atmosphere. The reaction mixture was then concentrated under reduced pressure to give a residue. The residue was purified by Prep-TLC (SiO2, Petroleum ether/Ethyl acetate=1:1) to afford the corresponding ether compound (45 mg, 70% yield) as a colorless solid.

LCMS: Rt=0.646 min; (ESI positive ion) m/z: 606.2 (M+H)+(calculated: 606.18).

To a solution of the above ether compound (40 mg, 66.06 μmol, 1 eq) in MeOH (1 mL) was added NH3·H2O (455.00 mg, 3.64 mmol, 0.5 mL, 28% purity, 55.03 eq). The mixture was stirred at 25° C. for 0.5 hr and then concentrated under vacuum. The residue was purified by Prep-HPLC (column: Waters xbridge 150×25 mm 10 μm; mobile phase: [water (NH4HCO3)-ACN]; B %: 12%-42%, 8 min) to afford Compound 229 (10.23 mg, 36% yield) as a white solid. LCMS: Rt=0.411 min; (ESI positive ion) m/z: 418.0 (M+H)+(calculated: 418,13).

1H NMR (400 MHz, DMSO-d6) δ=8.63 (s, 1H), 8.56 (s, 1H), 8.27 (d, J=8.8 Hz, 2H), 7.76 (d, J=8.6 Hz, 2H), 5.95 (d, J=5.4 Hz, 1H), 5.80 (s, 2H), 5.46 (d, J=5.8 Hz, 1H), 5.21 (d, J=5.2 Hz, 1H), 4.70 (q, J=5.4 Hz, 1H), 4.48 (t, J=5.2 Hz, 1H), 4.10 (q, J=4.8 Hz, 1H), 4.04-3.99 (m, 1H), 3.51-3.43 (m, 2H), 1.89-1.80 (m, 2H).

### Compound 232:

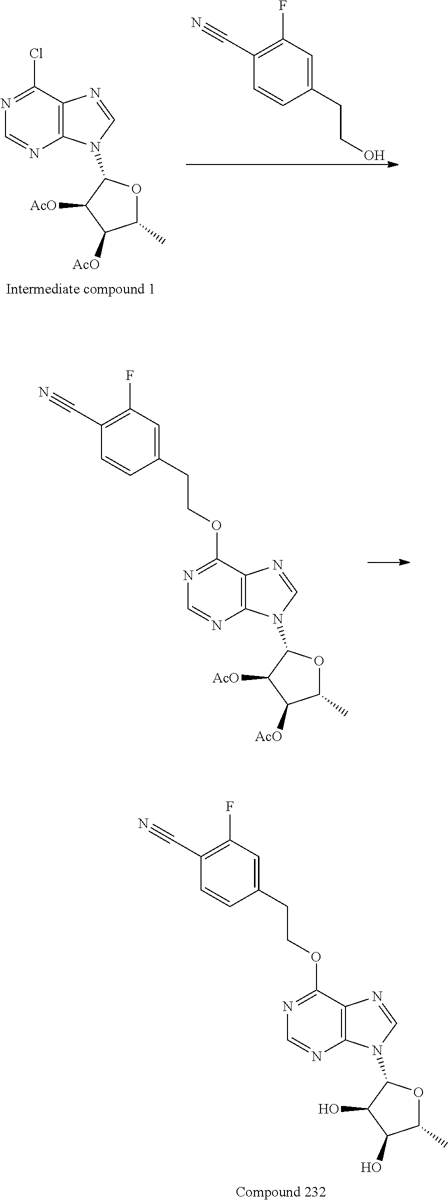 [SMILES: CC(=O)O[C@@H]1[C@H](OC(C)=O)[C@@H](C)O[C@H]1n1cnc2c(Cl)ncnc21 ; N#Cc1ccc(CCO)cc1F ; CC(=O)O[C@@H]1[C@H](OC(C)=O)[C@@H](C)O[C@H]1n1cnc2c(OCCc3ccc(C#N)c(F)c3)ncnc21 ; C[C@H]1O[C@@H](n2cnc3c(OCCc4ccc(C#N)c(F)c4)ncnc32)[C@H](O)[C@@H]1O]

Compound 232 (51.09 mg, 46% yield) was synthesized from Intermediate Compound 1 and 2-fluoro-4-(2-hydroxyethyl)benzonitrile following the protocols described for Compound 188.

LCMS: Rt=0.803 min; (ESI positive ion) m/z: 400.1 (M+H)+(calculated: 400.13).

1H NMR (400 MHz, DMSO-d6) δ=8.57 (s, 1H), 8.54 (s, 1H), 7.87 (t, J=7.5 Hz, 1H), 7.57 (d, J=9.7 Hz, 1H), 7.41 (dd, J=1.3, 8.0 Hz, 1H), 5.93 (d, J=5.0 Hz, 1H), 5.48 (d, J=5.7 Hz, 1H), 5.22 (d, J=5.0 Hz, 1H), 4.82 (t, J=6.5 Hz, 2H), 4.67 (q, J=5.1 Hz, 1H), 4.03-3.95 (m, 2H), 3.27 (t, J=6.5 Hz, 2H), 1.31 (d, J=6.2 Hz, 3H).

### Compound 233:

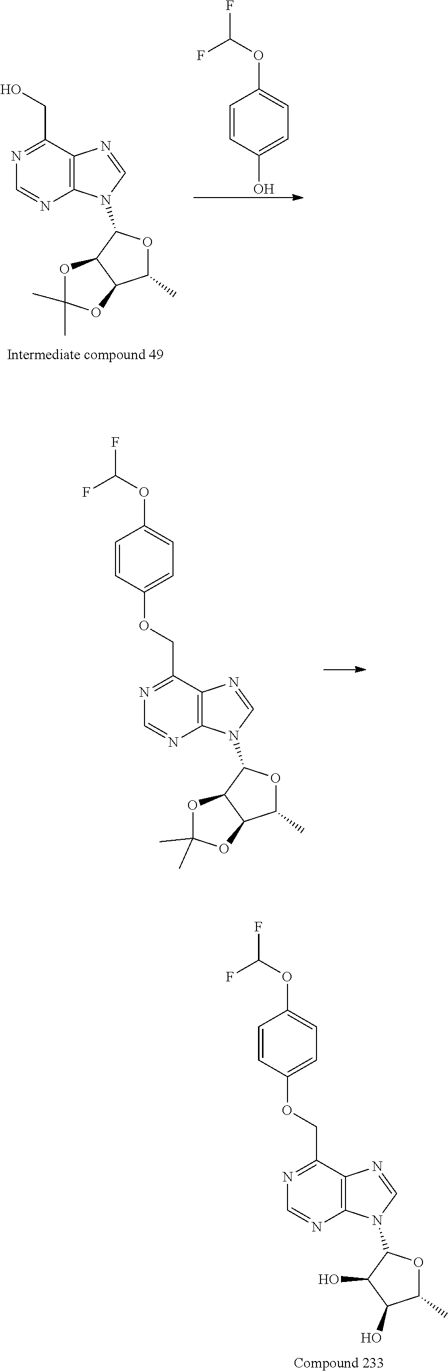

To a mixture of Intermediate Compound 49 (30 mg, 97.94 μmol, 1 eq), 4-(difluoromethoxy)phenol (15.68 mg, 97.94 μmol, 1 eq) and triphenylphosphine (28.26 mg, 107.73 mol, 1.1 eq) in THE (3 mL) was added DEAD (18.76 mg, 107.73 μmol, 19.59 μL, 1.1 eq) at 0° C. The mixture was stirred at 20° C. for 16 hr. The mixture was concentrated under vacuum and the residue was purified by Prep-TLC (Petroleum ether/Ethyl acetate=1/1) to afford the corresponding ether compound (25 mg, 51% yield) as a white solid.

A mixture of the above ether compound (25 mg, 55.75 μmol, 1 eq) in formic acid (2 mL) and water (0.5 mL) was stirred at 20° C. for 1 hr. The mixture was concentrated under vacuum and the residue was purified by Prep-HPLC (column: Unisil 3-100 C18 Ultra 150×50 mm×3 μm; mobile phase:[water(0.225% FA)ACN]; B %: 20%-50%, 10 min) to afford Compound 233 (7.08 mg, 31% yield) as a white solid.

LCMS: Rt=0.806 min; (ESI positive ion) m/z: 408.9 (M+H)+(calculated: 409.12).

1H NMR (400 MHz, DMSO-d6) δ=8.96 (s, 1H), 8.84 (s, 1H), 7.27-7.09 (m, 4H), 7.08-6.89 (m, 1H), 6.89 (s, 1H), 6.00 (d, J=5.0 Hz, 1H), 5.61-5.51 (m, 3H), 5.29 (br s, 1H), 4.75 (br s, 1H), 4.05-3.99 (m, 2H), 1.33 (d, J=6.2 Hz, 3H).

### Compound 237:

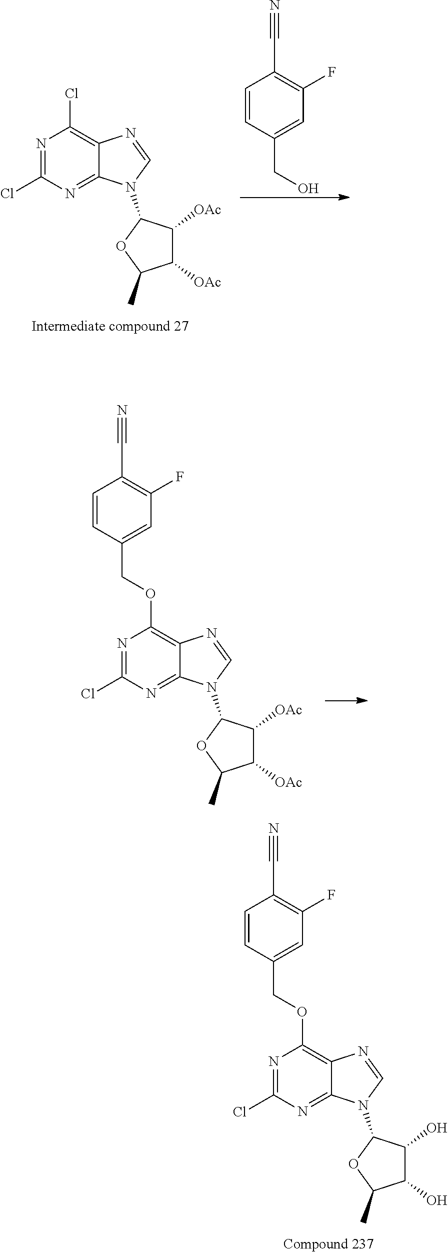

To a solution of Intermediate Compound 27 (120 mg, 308.33 μmol) in toluene (5 mL) was added 2-fluoro-4-(hydroxymethyl)benzonitrile (44.27 mg, 292.92 μmol), Cs2CO3 (251.15 mg, 770.83 μmol), Xantphos (35.68 mg, 61.67 μmol) and Pd2(dba)3 (28.23 mg, 30.83 mol). The mixture was degassed and purged with N2 3 times and then stirred at 80° C. for 1 hr under N2 atmosphere. The reaction mixture was filtered and concentrated under reduced pressure to give a residue, which was purified by Prep-TLC (SiO2, Petroleum ether:Ethyl acetate=1:1) to afford the corresponding ether compound (79 mg, 50% yield) as a yellow oil.

LCMS: Rt=0.591 min; (ESI positive ion) m/z: 526.0 (M+Na)+(calculated: 526,10).

To a solution of the above ether compound (79 mg, 156.79 μmol) in MeOH (1 mL) was added NH3·H2O (963.35 mg, 7.70 mmol, 1.06 mL, 28% purity). The mixture was stirred at 25° C. for 1 hr and then concentrated to give a residue. The residue was dissolved in DMSO and purified by Prep-HPLC (Phenomenex column C18 150×25 mm×10 μm; mobile phase: [water(FA)-ACN]; B %: 29%-59%, 2 min). The solution was then lyophilized to give Compound 237 (22.62 mg, 34% yield) as a white solid.

LCMS: Rt=0.495 min; (ESI positive ion) m/z: 420.1 (M+H)+(calculated: 420.08).

1H NMR (400 MHz, DMSO-d6) δ=8.66 (s, 1H), 8.03-7.97 (m, 1H), 7.68 (d, J=10.2 Hz, 1H), 7.58-7.54 (m, 1H), 5.89 (d, J=5.0 Hz, 1H), 5.72 (s, 2H), 5.51 (d, J=5.8 Hz, 1H), 5.24 (d, J=5.4 Hz, 1H), 4.62 (q, J=5.2 Hz, 1H), 4.02 (dd, J=4.6, 6.4 Hz, 1H), 3.97 (q, J=5.0 Hz, 1H), 1.33 (d, J=6.4 Hz, 3H).

### Compound 239:

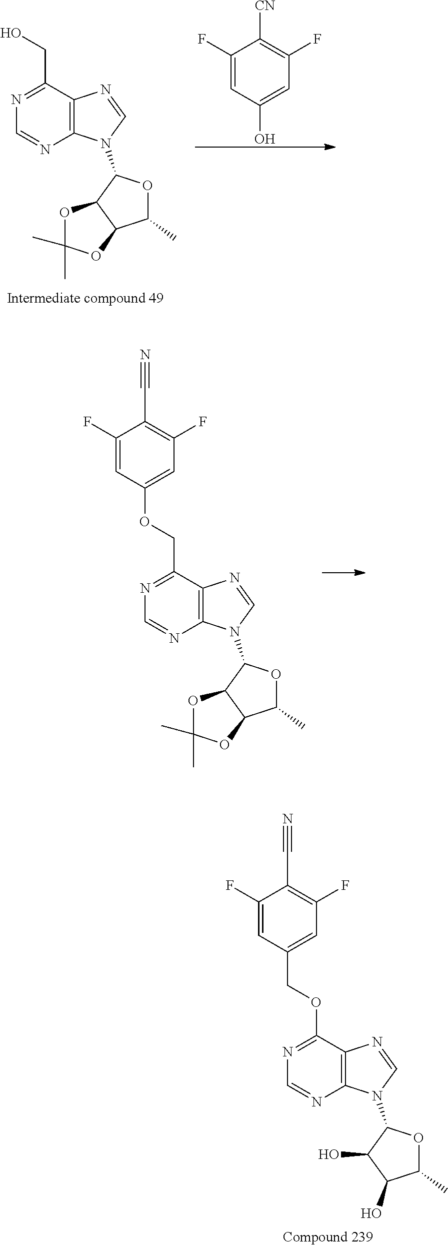 [SMILES: C[C@H]1O[C@@H](n2cnc3c(CO)ncnc32)[C@@H]2OC(C)(C)O[C@@H]21 ; N#Cc1c(F)cc(O)cc1F ; C[C@H]1O[C@@H](n2cnc3c(COc4cc(F)c(C#N)c(F)c4)ncnc32)[C@@H]2OC(C)(C)O[C@@H]21 ; C[C@H]1O[C@@H](n2cnc3c(OCc4cc(F)c(C#N)c(F)c4)ncnc32)[C@H](O)[C@@H]1O]

Compound 239 (7.45 mg, 41% yield) was synthesized from Intermediate Compound 49 and 2,6-difluoro-4-hydroxybenzonitrile following the protocols described for Compound 188.

LCMS: Rt=0.808 min; (ESI positive ion) m/z: 403.9 (M+H)+(calculated: 404.11).

1H NMR (400 MHz, DMSO-d6) δ=8.97 (s, 1H), 8.87 (s, 1H), 7.28-7.22 (m, 2H), 6.00 (d, J=5.0 Hz, 1H), 5.75 (s, 2H), 5.52 (d, J=5.6 Hz, 1H), 5.24 (d, J=4.8 Hz, 1H), 4.74 (q, J=4.8 Hz, 1H), 4.09-3.98 (m, 2H), 1.33 (d, J=6.2 Hz, 3H).

### Compound 240:

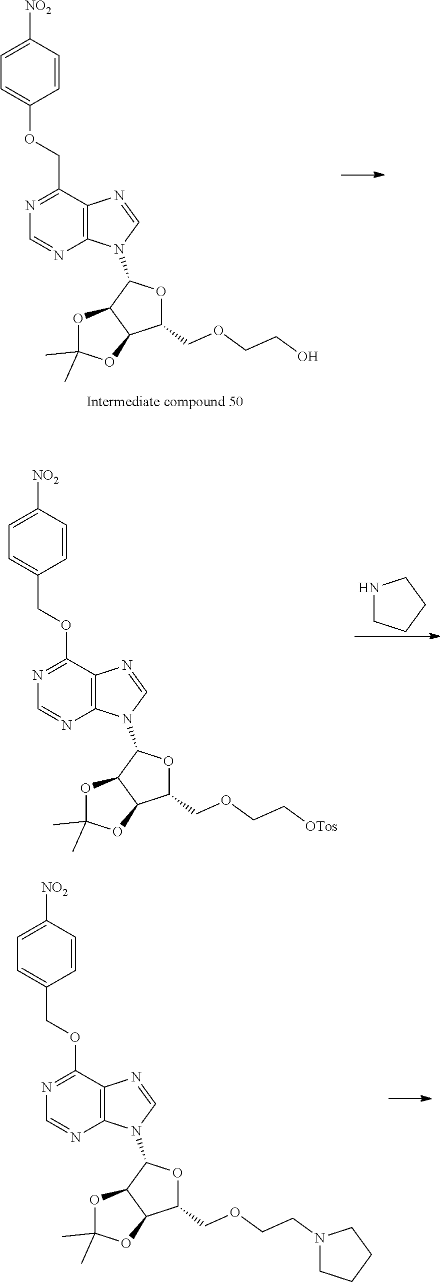

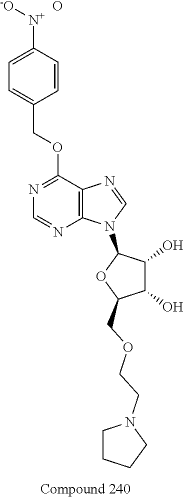

To a solution of Intermediate Compound 50 (40 mg, 82.06 μmol, 1 eq), TEA (16.61 mg, 164.12 μmol, 22.84 μL, 2 eq) and DMAP (1.00 mg, 8.21 μmol, 0.1 eq) in DCM (1 mL) was added p-toluenesulfonyl chloride (18.77 mg, 98.47 μmol, 1.2 eq) at 0° C. The mixture was stirred at 25° C. for 2 hr. The mixture was concentrated under vacuum and the residue was purified by Prep-TLC (Petroleum ether/Ethyl acetate=1/1) to afford the corresponding tosylate compound (33 mg, 59% yield) as a yellow oil.

LCMS: Rt=0.572 min; (ESI positive ion) m/z: 642.1 (M+H)+(calculated: 642.18).

A mixture of the above tosylate compound (33 mg, 51.43 μmol, 1 eq), pyrrolidine (10.97 mg, 154.29 μmol, 12.88 μL, 3 eq) and K2CO3 (14.22 mg, 102.86 μmol, 2 eq) in dioxane (1 mL) was stirred at 50° C. for 16 hr. The mixture was filtered and concentrated under vacuum. The resultant residue was purified by Prep-TLC (DCM/MeOH=10/1) to afford the corresponding amine compound (18 mg, 64.74% yield) as a colorless oil.

A solution of the above amine compound (18 mg, 33.30 μmol, 1 eq) in water (0.2 mL) and formic acid (0.8 mL) was stirred at 25° C. for 1 hr. The mixture was concentrated under reduced pressure to give a residue. The residue was purified by Prep-HPLC (column: Phenomenex luna C18 150×25 mm×10 μm; mobile phase:[water(FA)-ACN]; B %: 5%-35%, 10 min) to afford Compound 240 (9 mg, 54% yield, formic acid salt) as a white solid.

LCMS: Rt=0.332 min; (ESI positive ion) m/z: 501.2 (M+H)+(calculated: 501.20).

1H NMR (400 MHz, DMSO-d6) δ=8.70 (s, 1H), 8.26 (d, J=8.8 Hz, 2H), 8.22 (s, 1H), 7.76 (d, J=8.4 Hz, 2H), 6.03 (d, J=5.6 Hz, 1H), 5.80 (s, 2H), 4.63-4.60 (m, 1H), 4.56-4.32 (m, 1H), 4.20-4.18 (m, 1H), 3.68-3.52 (m, 6H), 2.68-2.57 (m, 3H), 2.56-2.52 (m, 3H), 1.72-1.69 (m, 4H).

### Compound 243:

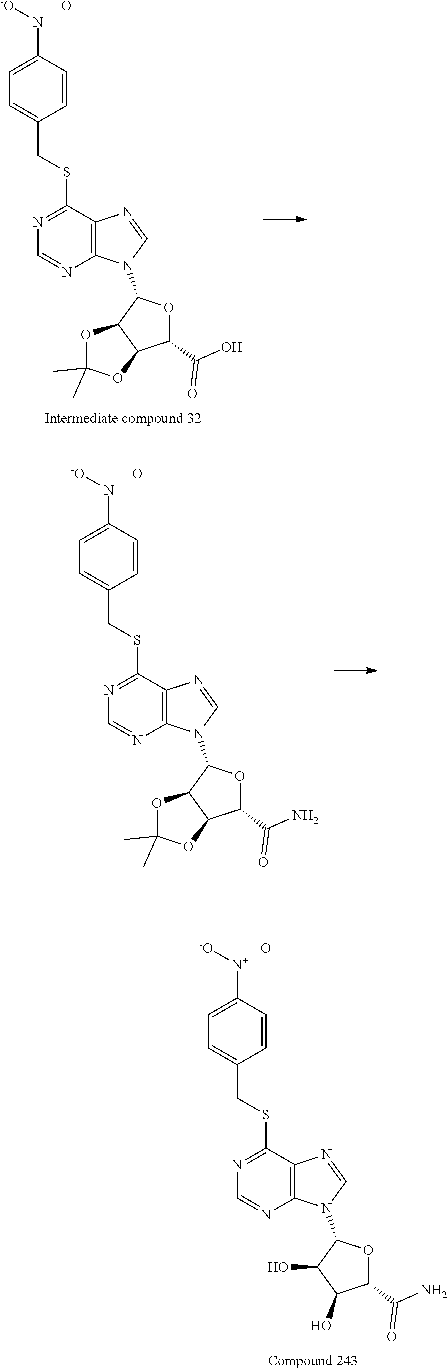

To a solution of Intermediate Compound 32 (124 mg, 261.90 μmol, 1 eq) in DMF (2 mL) were added benzotriazol-1-yloxytripyrrolidinophosphonium hexafluorophosphate (272.58 mg, 523.81 μmol, 2 eq), DIEA (101.54 mg, 785.71 μmol, 136.85 μL, 3 eq) and ammonium 1-oxidobenzotriazole (79.70 mg, 523.81 μmol, 2 eq). The mixture was stirred at 25° C. for 16 hr. The reaction mixture was then filtered and the filtrate was purified by Prep-HPLC (column: Phenomenex Gemini-NX C18 75×30 mm×3 μm; mobile phase: [water(0.225% FA)-ACN]; B %: 32%-62%, 7 min). The purified solution was lyophilized to afford the corresponding amide compound (80 mg, 65% yield) as a yellow solid.

LCMS: Rt=0.881 min; (ESI positive ion) m/z: 473.3 (M+H)+(calculated: 473.12).

1H NMR (400 MHz, DMSO-d6) δ=8.73 (s, 1H), 8.61 (s, 1H), 8.17 (d, J=8.8 Hz, 2H), 7.75 (d, J=8.9 Hz, 2H), 7.08 (br d, J=18.4 Hz, 2H), 6.42 (d, J=1.1 Hz, 1H), 5.47 (dd, J=1.1, 6.1 Hz, 1H), 5.43-5.39 (m, 1H), 4.78 (s, 2H), 4.56 (d, J=2.1 Hz, 1H), 1.54 (s, 3H), 1.34 (s, 3H).

To a mixture of the above amide compound (75 mg, 158.74 μmol, 1 eq) in water (0.3 mL) was added TFA (1.85 g, 16.21 mmol, 1.2 mL, 102.10 eq) at 0° C. The mixture was stirred at 20° C. for 2 hr. The reaction mixture was then diluted with water (20 mL) and lyophilized to give a residue. The residue was purified by Prep-HPLC (column: Phenomenex Gemini-NX C18 75×30 mm×3 μm; mobile phase: [water(0.225% FA)-ACN]; B %: 20%-50%, 7 min). The purified solution was lyophilized to afford Compound 243 (23.87 mg, 35% yield) as a white solid.

LCMS: Rt=0.778 min; (ESI positive ion) m/z: 433.0 (M+H)+(calculated: 433.09).

1H NMR (400 MHz, DMSO-d6) δ=8.86 (s, 1H), 8.77 (s, 1H), 8.17 (d, J=8.7 Hz, 2H), 7.82 (s, 1H), 7.75 (d, J=8.7 Hz, 2H), 7.47 (s, 1H), 6.08 (d, J=6.7 Hz, 1H), 5.73 (br d, J=4.5 Hz, 1H), 5.65 (br d, J=5.9 Hz, 1H), 4.81 (s, 2H), 4.67-4.59 (m, 1H), 4.32 (d, J=2.2 Hz, 1H), 4.23 (br s, 1H).

### Compound 244:

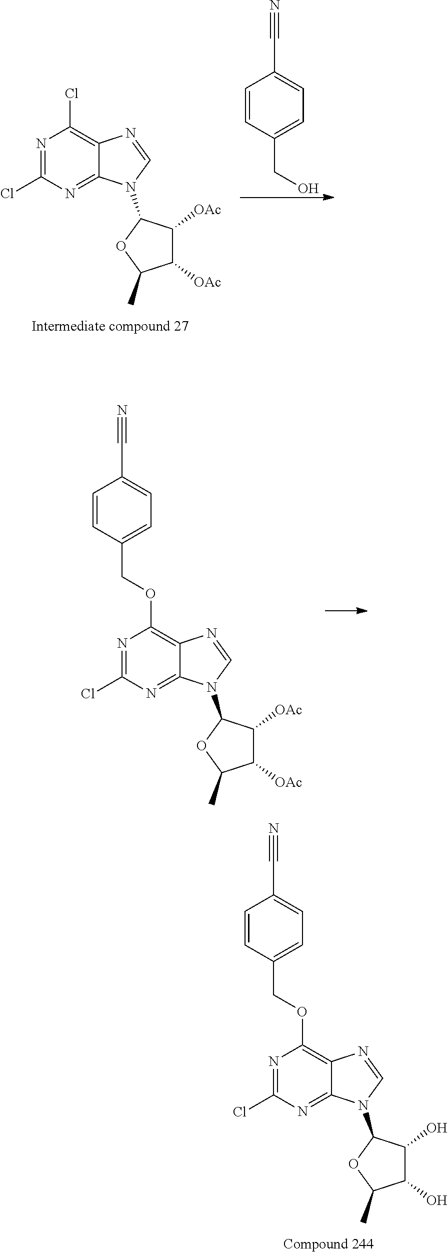

To a solution of Intermediate Compound 27 (150 mg, 385.42 μmol) in toluene (8 mL) was added 4-(hydroxymethyl)benzonitrile (48.75 mg, 366.14 μmol), Cs2CO3 (313.94 mg, 963.54 μmol), Xantphos (44.60 mg, 77.08 μmol) and Pd2(dba)3 (35.29 mg, 38.54 μmol). The mixture was degassed and purged with N2 3 times and stirred at 80° C. for 1 hr. The reaction mixture was filtered and concentrated to give a residue, which was purified by Prep-TLC (SiO2, Ethyl acetate:Petroleum ether=2:1) to afford the corresponding ether compound (95 mg, 49% yield) as a yellow oil.

LCMS: Rt=0.587 min; (ESI positive ion) m/z: 486.2 (M+H)+(calculated: 486,11) To a solution of the above ether compound (95 mg, 195.52 μmol) in MeOH (1.2 mL) was added NH3·H2O (1.20 g, 9.60 mmol, 1.32 mL, 28% purity). The mixture was stirred at 25° C. for 1 hr. The reaction mixture was then concentrated to give a residue, which was dissolved in DMSO and purified by Prep-HPLC (Phenomenex column C18 150×25 mm×10 μm; mobile phase: [water (FA)-ACN]; B %: 26%-56%, 10 min) to afford Compound 244 (46.98 mg, 59% yield) as a white solid.

LCMS: Rt=0.479 min; (ESI positive ion) m/z: 402.0 (M+H)+(calculated: 402.09).

1H NMR (400 MHz, DMSO-d6) δ=8.65 (s, 1H), 7.90 (d, J=8.4 Hz, 2H), 7.71 (d, J=8.2 Hz, 2H), 5.89 (d, J=5.0 Hz, 1H), 5.71 (s, 2H), 5.50 (d, J=5.8 Hz, 1H), 5.24 (d, J=5.4 Hz, 1H), 4.65-4.59 (m, 1H), 4.04-4.00 (m, 1H), 3.96 (q, J=5.0 Hz, 1H), 1.32 (d, J=6.4 Hz, 3H).

### Compound 247:

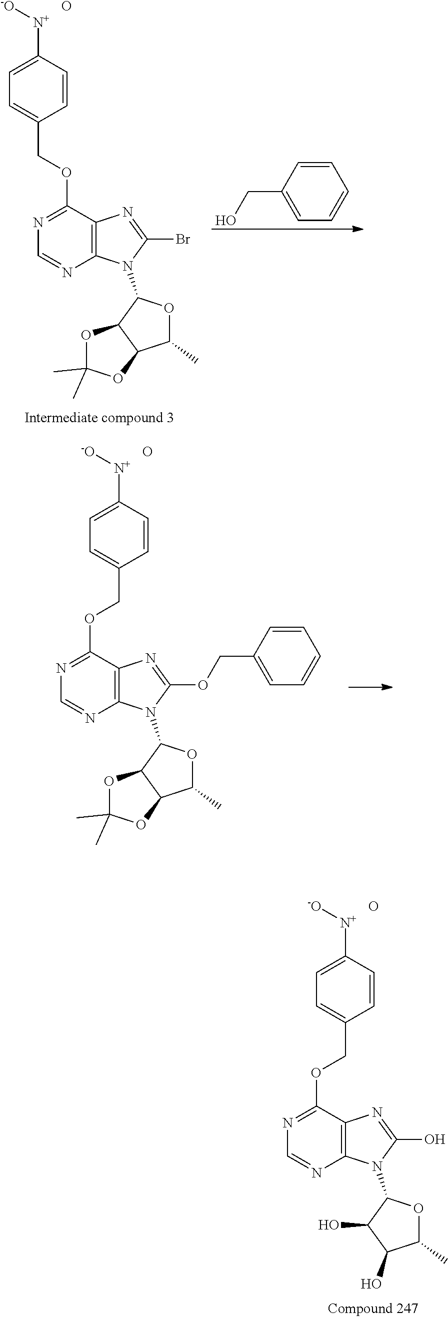

A mixture of Intermediate Compound 3 (150 mg, 296.26 μmol, 1 eq), benzyl alcohol (48.06 mg, 444.40 μmol, 46.21 μL, 1.5 eq), Pd2(dba)3 (27.13 mg, 29.63 μmol, 0.1 eq), Xantphos (34.28 mg, 59.25 μmol, 0.2 eq) and Cs2CO3 (241.32 mg, 740.66 μmol, 2.5 eq) in toluene (2 mL) was degassed and purged with N2 3 times. Then mixture was stirred at 80° C. for 1 hr under N2 atmosphere. The reaction mixture then concentrated under reduced pressure to give a residue, which was purified by Prep-TLC (SiO2, PE:EA=3:1) to afford the corresponding ether compound (68 mg, 43% yield) as a yellow oil.

LCMS: Rt=0.575 min; (ESI positive ion) m/z: 534.2 (M+H)+(calculated: 534.19).

A solution of the above ether compound (57 mg, 106.84 μmol, 1 eq) in FA/H2O=4/1 (V/V) (2 mL) was stirred at 0° C. for 3 hr. NH3·H2O was added to the reaction mixture at 0° C. until the pH reached 7. The mixture was then diluted with water (20 mL×3) and extracted with ethyl acetate (20 mL×3). The combined organic layers were concentrated under reduced pressure to give a residue, which was purified by Prep-HPLC (column: Phenomenex C18 150×25 mm×10 μm; mobile phase: [water (NH4HCO3)-ACN]; B %: 16%-46%, 8 min) to afford Compound 247 (23.31 mg, 54% yield) as a white solid.

LCMS: Rt=0.418 min; (ESI positive ion) m/z: 404.1 (M+H)+(calculated: 404.11).

1H NMR (400 MHz, DMSO-d6) δ=11.78-11.56 (m, 1H), 8.33 (s, 1H), 8.25 (d, J=8.8 Hz, 2H), 7.76 (d, J=8.6 Hz, 2H), 5.70 (s, 2H), 5.67 (d, J=4.6 Hz, 1H), 5.23 (d, J=5.4 Hz, 1H), 5.01 (d, J=5.8 Hz, 1H), 4.85 (q, J=5.2 Hz, 1H), 4.09-4.04 (m, 1H), 3.86 (t, J=6.0 Hz, 1H), 1.24 (d, J=6.4 Hz, 3H).

### Compound 248:

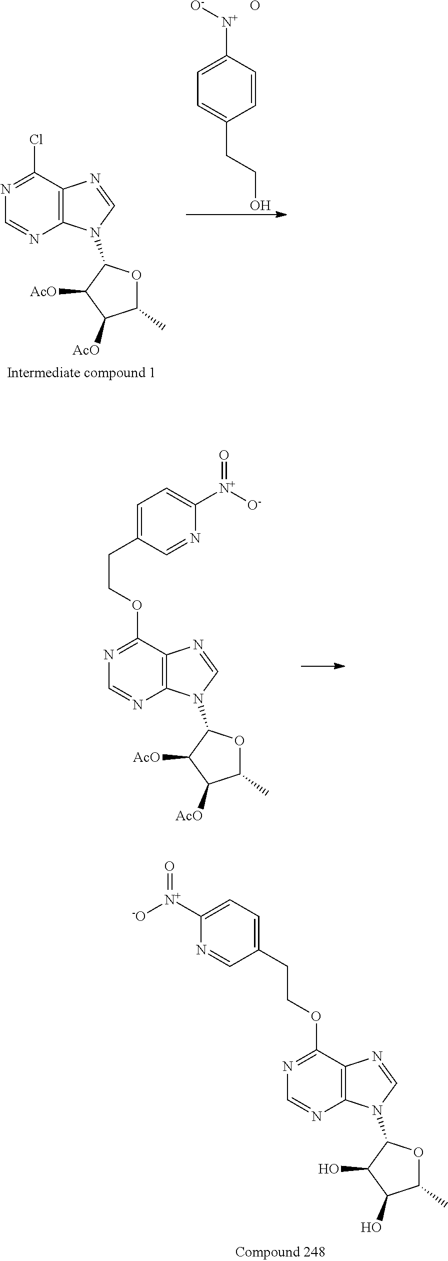

Compound 248 (22.97 mg, 15% yield) was synthesized from Intermediate Compound 1 and 2-(6-nitropyridin-3-yl)ethan-1-ol following the protocols described for Compound 188.

LCMS: Rt=0.761 min; (ESI positive ion) m/z: 403.0 (M+H)+(calculated: 403.13).

1H NMR (400 MHz, DMSO-d6) δ=8.66 (d, J=1.8 Hz, 1H), 8.57 (s, 1H), 8.54 (s, 1H), 8.30-8.26 (m, 1H), 8.25-8.19 (m, 1H), 5.93 (d, J=5.0 Hz, 1H), 5.48 (br d, J=4.8 Hz, 1H), 5.21 (br s, 1H), 4.87 (t, J=6.4 Hz, 2H), 4.67 (br d, J=4.0 Hz, 1H), 4.02-3.96 (m, 2H), 3.38-3.35 (m, 2H), 1.31 (d, J=6.0 Hz, 3H).

### Compound 253:

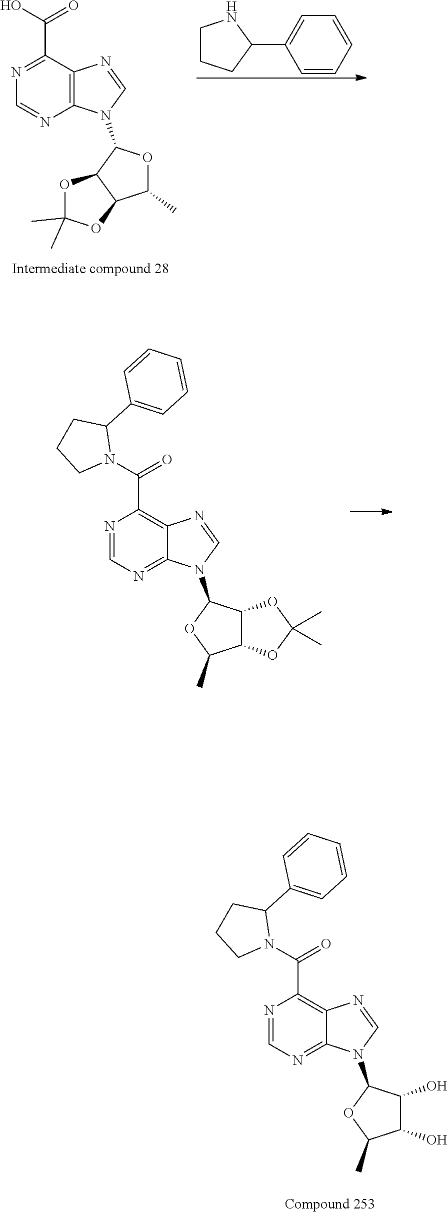 [SMILES: C[C@H]1O[C@@H](n2cnc3c(C(=O)O)ncnc32)[C@@H]2OC(C)(C)O[C@@H]21 ; c1ccc(C2CCCN2)cc1 ; C[C@H]1O[C@@H](n2cnc3c(C(=O)N4CCCC4c4ccccc4)ncnc32)[C@@H]2OC(C)(C)O[C@@H]21 ; C[C@H]1O[C@@H](n2cnc3c(C(=O)N4CCCC4c4ccccc4)ncnc32)[C@H](O)[C@@H]1O]

To a solution of Intermediate Compound 28 (100 mg, 312.21 μmol) in THE (10 mL) was added 2-phenylpyrrolidine (55.15 mg, 374.65 μmol), T3P (496.69 mg, 780.52 μmol, 464.20 μL, 50% purity) and DIEA (201.75 mg, 1.56 mmol, 271.90 μL) at 0° C. The mixture was stirred at 25° C. for 1 hr. The reaction mixture was concentrated under vacuum to give a residue, which was purified by Prep-TLC (SiO2, Ethyl acetate:Petroleum ether=3:1) to afford the corresponding amide compound (120 mg, 62% yield) as a colorless gum.

LCMS: Rt=0.512 min; (ESI positive ion) m/z: 450.1 (M+H)+(calculated: 450.21).

A solution of the above amide compound (115 mg, 255.84 μmol) in FA (4 mL) and H2O (1 mL) was stirred at 0° C. for 6 hr. NH3·H2O was added to reaction mixture at 0° C. until the pH reached 7. The mixture was then filtered and concentrated under reduced pressure to give a residue. The residue was dissolved in MeOH and purified by Prep-HPLC (Phenomenex C18 150×25 mm×10 μm; mobile phase: [water (NH4HCO3)-ACN]; B %: 5%-35%, 8 min) to afford the mixture of diastereomers of Compound 253 (25.04 mg, 24% yield) as a gray gum.

LCMS: Rt=0.680 and 0.721 min; (ESI positive ion) m/z: 410.2 (M+H)+(calculated: 410.18).

1H NMR (400 MHz, DMSO-d6) δ=9.04 (s, 1H), 8.93 (d, J=2.0 Hz, 1H), 8.72 (s, 1H), 8.64 (d, J=6.0 Hz, 1H), 7.45-7.42 (m, 2H), 7.39-7.35 (m, 2H), 7.28-7.24 (m, 1H), 6.96-6.88 (m, 2H), 6.76-6.72 (m, 1H), 6.04 (dd, J=1.4, 4.8 Hz, 1H), 5.87 (dd, J=1.6, 4.6 Hz, 1H), 5.54 (dd, J=1.0, 5.6 Hz, 1H), 5.48 (dd, J=2.8, 5.6 Hz, 1H), 5.32 (dd, J=3.2, 7.8 Hz, 1H), 5.24 (d, J=5.2 Hz, 1H), 5.22-5.18 (m, 1H), 5.03-4.95 (m, 1H), 4.78-4.71 (m, 1H), 4.58-4.52 (m, 1H), 4.07-3.80 (m, 5H), 3.63-3.57 (m, 1H), 2.43-2.38 (m, 1H), 1.96 (br t, J=6.8 Hz, 1H), 1.85-1.78 (m, 4H), 1.35 (d, J=6.2 Hz, 3H), 1.32-1.30 (m, 2H).

### Compound 254:

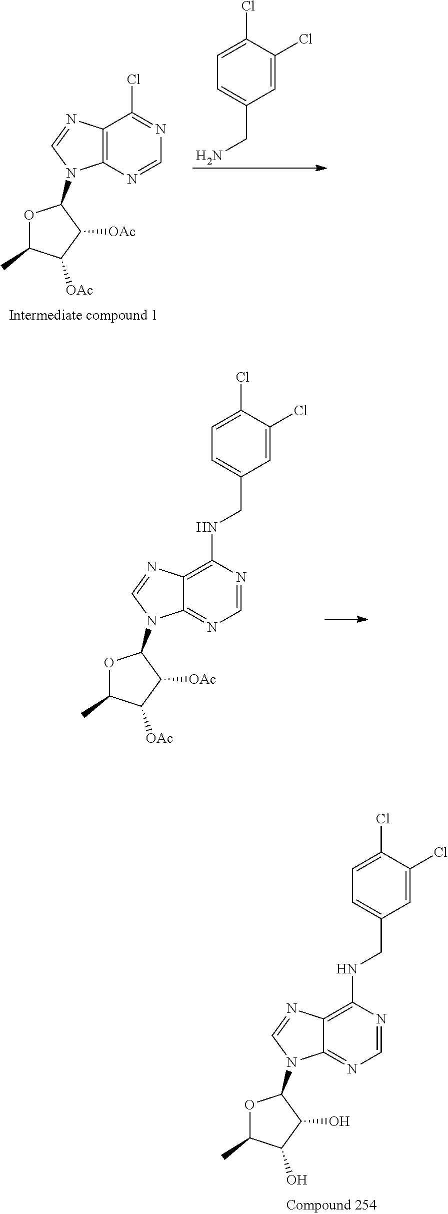 [SMILES: CC(=O)O[C@@H]1[C@H](OC(C)=O)[C@@H](C)O[C@H]1n1cnc2c(Cl)ncnc21 ; NCc1ccc(Cl)c(Cl)c1 ; CC(=O)O[C@@H]1[C@H](OC(C)=O)[C@@H](C)O[C@H]1n1cnc2c(NCc3ccc(Cl)c(Cl)c3)ncnc21 ; C[C@H]1O[C@@H](n2cnc3c(NCc4ccc(Cl)c(Cl)c4)ncnc32)[C@H](O)[C@@H]1O]

To a solution of Intermediate Compound 1 (200 mg, 563.79 μmol, 1 eq) in EtOH (2 mL) was added TEA (285.24 mg, 2.82 mmol, 392.36 μL, 5 eq) and (3,4-dichlorophenyl)methanamine (198.50 mg, 1.13 mmol, 150.38 μL, 2 eq). The mixture was stirred at 80° C. for 1 hr. The mixture was then concentrated under reduced pressure to give the corresponding amine (300 mg), which was used in next step without further purification.

To a solution of the above amine compound (300 mg, 606.89 μmol, 1 eq) in MeOH (3 mL) was added NH3·H2O (16.25 g, 129.83 mmol, 17.86 mL, 28% purity, 213.93 eq). The mixture was stirred at 50° C. for 0.5 hr. The reaction mixture was then concentrated under reduced pressure to give a crude product. The crude product was triturated with MeOH at 10° C. for 10 min and filtered. Water (10 mL) was added to the filtered cake and the solution was lyophilized to give Compound 254 (45 mg, 18% yield) as a white solid.

LCMS: Rt=0.577 min; (ESI positive ion) m/z: 410.0 (M+H)+(calculated: 410.07).

1H NMR (400 MHz, CDCl₃) δ=8.34 (s, 1H), 8.01 (s, 1H), 7.48 (s, 1H), 7.43-7.38 (m, 1H), 7.26-7.20 (m, 1H), 6.46 (s, 1H), 5.93-5.87 (m, 1H), 4.91 (s, 2H), 4.58-4.50 (m, 1H), 4.49-4.38 (m, 1H), 4.21-4.11 (m, 1H), 1.40 (d, J=6.8 Hz, 3H).

### Compound 256:

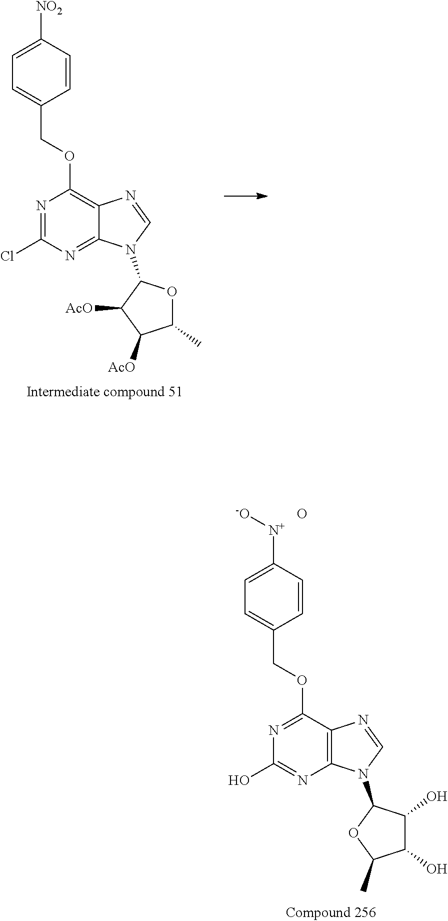

To a solution of Intermediate Compound 51 (200 mg, 395.36 μmol, 1 eq) in water (2 mL) was added hydrogen peroxide (89.65 mg, 790.73 μmol, 6.59 μL, 30% purity, 2 eq) and lithium hydroxide monohydrate (49.77 mg, 1.19 mmol, 3 eq). The mixture was stirred at 60° C. for 12 hr. To the reaction mixture was added a solution of 1N HCl to adjust the pH to less than 8. Then water (10 mL) was added to the mixture and the mixture was extracted with ethyl acetate (5 mL×3). The organic layer was washed with brine and the solution was concentrated to give a residue. The residue was purified by Prep-TLC (SiO2, Petroleum ether:Ethyl acetate=0:1, Rf=0.15) and Prep-HPLC (column: Phenomenex luna C18 150×25 mm×10 μm; mobile phase: [water(FA)-ACN]; B %: 13%-43%, 10 min). The purified solution was lyophilized to afford Compound 256 (5.51 mg, 3% yield) as a white solid.

LCMS: Rt=0.666 min; (ESI positive ion) m/z: 404.2 (M+H)+(calculated: 404.11).

1H NMR (400 MHz, MeOD-d4) δ=8.26 (d, J=8.6 Hz, 2H), 8.13 (s, 1H), 7.78 (d, J=8.6 Hz, 2H), 5.89 (d, J=4.6 Hz, 1H), 5.73 (s, 2H), 4.65-4.63 (m, 1H), 4.16-4.08 (m, 1H), 4.08-4.00 (m, 1H), 1.41 (d, J=6.4 Hz, 3H).

### Compound 258:

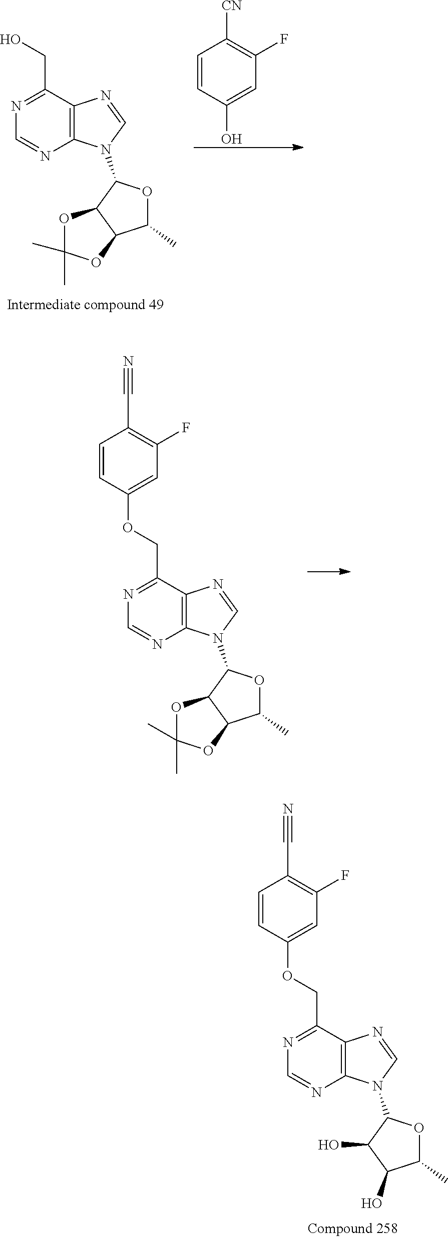

Compound 258 (6.20 mg, 34%) was synthesized from Intermediate Compound 49 and 2-fluoro-4-hydroxybenzonitrile following the protocols described for Compound 233.

LCMS: Rt=0.779 min; (ESI positive ion) m/z: 385.9 (M+H)+(calculated: 386.12).

1H NMR (400 MHz, DMSO-d6) δ=8.96 (s, 1H), 8.86 (s, 1H), 7.83 (t, J=8.4 Hz, 1H), 7.33 (dd, J=2.4, 11.8 Hz, 1H), 7.08 (dd, J=2.4, 8.6 Hz, 1H), 6.00 (d, J=5.0 Hz, 1H), 5.72 (s, 2H), 5.54 (d, J=5.4 Hz, 1H), 5.26 (br d, J=4.6 Hz, 1H), 4.74 (q, J=4.8 Hz, 1H), 4.08-3.98 (m, 2H), 1.33 (d, J=6.2 Hz, 3H).

### Compound 259:

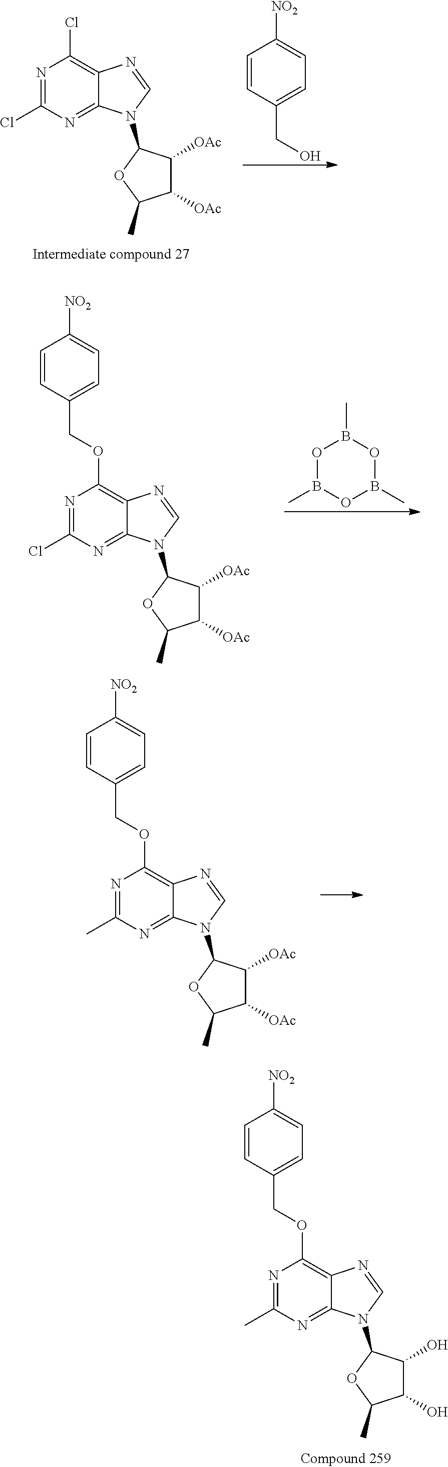

To a solution of Intermediate Compound 27 (300 mg, 770.83 μmol, 1 eq) in toluene (10 mL) was added Xantphos (89.20 mg, 154.17 μmol, 0.2 eq), Pd2(dba)3 (44.32 mg, 77.08 μmol, 0.1 eq), Cs2CO3 (627.88 mg, 1.93 mmol, 2.5 eq), and (4-nitrophenyl)methanol (118.04 mg, 770.83 μmol, 1 eq). The mixture was stirred at 80° C. for 1 hr. The mixture was filtered and concentrated under reduced pressure to give a residue. The residue was purified by Prep-TLC (SiO2, Petroleum ether:Ethyl acetate=9:1, Rf=0.3) to afford the corresponding ether compound (332 mg, crude) as a yellow oil.

LCMS: Rt=0.469 min; (ESI positive ion) m/z: 506.1 (M+H)+(calculated: 506.10).

To a solution of the above ether compound (180 mg, 355.83 μmol, 1 eq) in DMF (8 mL) was added 2,4,6-trimethyl-1,3,5,2,4,6-trioxatriborinane (446.68 mg, 1.78 mmol, 497.42 μL, 50% purity, 5 eq), Pd(dppf)Cl2·CH2Cl2 (29.06 mg, 35.58 μmol, 0.1 eq) and K2CO3 (147.53 mg, 1.07 mmol, 3 eq). The mixture was stirred at 80° C. for 12 hr. The mixture was then filtered and concentrated under reduced pressure to give a residue. The residue was purified by column chromatography (SiO2, Dichloromethane/Methanol=100/1 to 81/19, TLC Petroleum ether/Ethyl acetate=3/1, RF=0.4) and the isolated methylated compound was used directly in the next step.

To a solution of the above methylated compound (172.73 mg, 355.82 μmol, 1 eq) in MeOH (1 mL) was added NH3·H2O (910.00 mg, 7.27 mmol, 1 mL, 28% purity, 20.43 eq). The mixture was stirred at 25° C. for 0.5 hr. The mixture was filtered and concentrated under reduced pressure to give a residue. The crude product was dissolved in MeOH (2 mL) and then purified by Prep-HPLC (column: Phenomenex luna C18 150×25 mm×10 μm; mobile phase: [water (FA)-ACN]; B %: 28%-58%, 10 min) to afford Compound 259 (16.05 mg, 11% yield) as a white solid.

LCMS: Rt=0.360 min; (ESI positive ion) m/z: 402.1 (M+H)+(calculated: 402.13).

1H NMR (400 MHz, DMSO-d6) δ=8.20-8.19 (m, 1H), 7.47-7.44 (m, 2H), 7.40-7.36 (m, 2H), 7.35-7.31 (m, 1H), 6.88 (s, 2H), 5.71-5.68 (m, 1H), 5.53 (s, 2H), 5.28 (d, J=5.4 Hz, 1H), 5.04 (d, J=5.8 Hz, 2H), 4.14-4.11 (m, 1H), 3.89-3.84 (m, 1H), 1.27-1.25 (m, 3H).

### Compound 261:

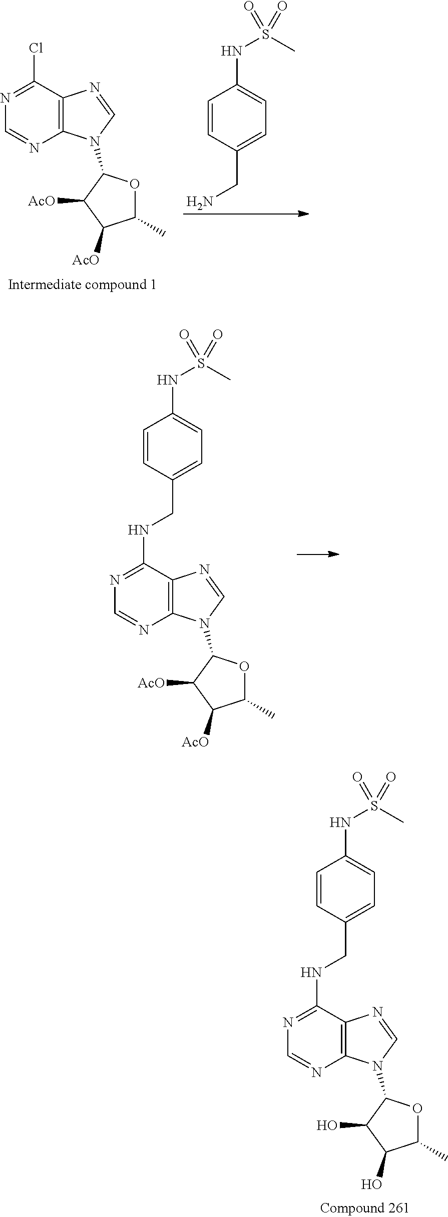 [SMILES: CC(=O)O[C@@H]1[C@H](OC(C)=O)[C@@H](C)O[C@H]1n1cnc2c(Cl)ncnc21 ; CS(=O)(=O)Nc1ccc(CN)cc1 ; CC(=O)O[C@@H]1[C@H](OC(C)=O)[C@@H](C)O[C@H]1n1cnc2c(NCc3ccc(NS(C)(=O)=O)cc3)ncnc21 ; C[C@H]1O[C@@H](n2cnc3c(NCc4ccc(NS(C)(=O)=O)cc4)ncnc32)[C@H](O)[C@@H]1O]

Compound 261 (52.38 mg, 56% yield) was synthesized from Intermediate Compound 1 and N-(4-(aminomethyl)phenyl)methanesulfonamide following the protocols described for Compound 254.

LCMS: Rt=0.654 min; (ESI positive ion) m/z: 435.0 (M+H)+(calculated: 435.14).

1H NMR (400 MHz, DMSO-d6) δ=9.77-9.48 (m, 1H), 8.34 (s, 2H), 8.21 (s, 1H), 7.30 (d, J=8.4 Hz, 2H), 7.12 (d, J=8.4 Hz, 2H), 5.85 (d, J=4.8 Hz, 1H), 5.46 (d, J=5.6 Hz, 1H), 5.18 (d, J=3.2 Hz, 1H), 4.66 (s, 3H), 4.03-3.92 (m, 2H), 2.92 (s, 3H), 1.30 (d, J=6.0 Hz, 3H).

### Compound 262:

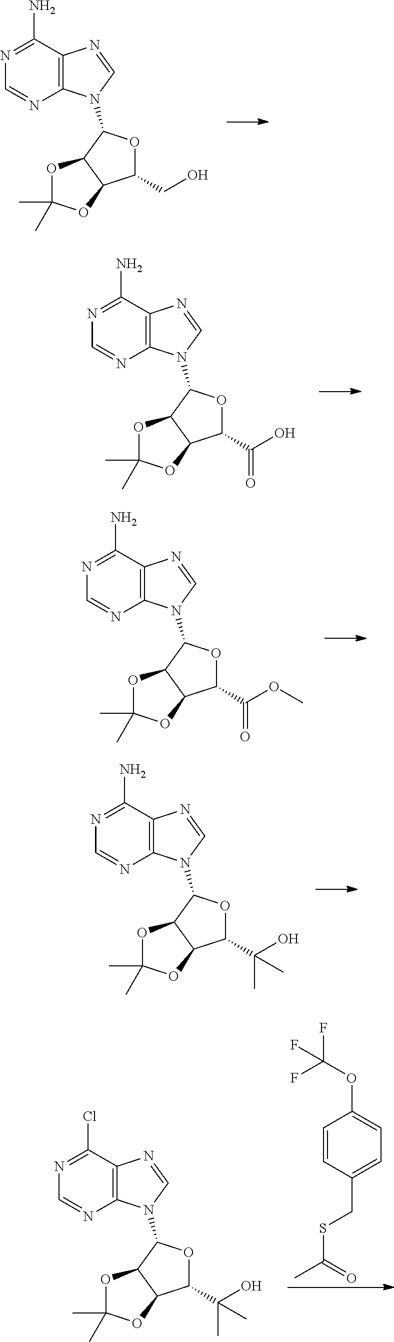

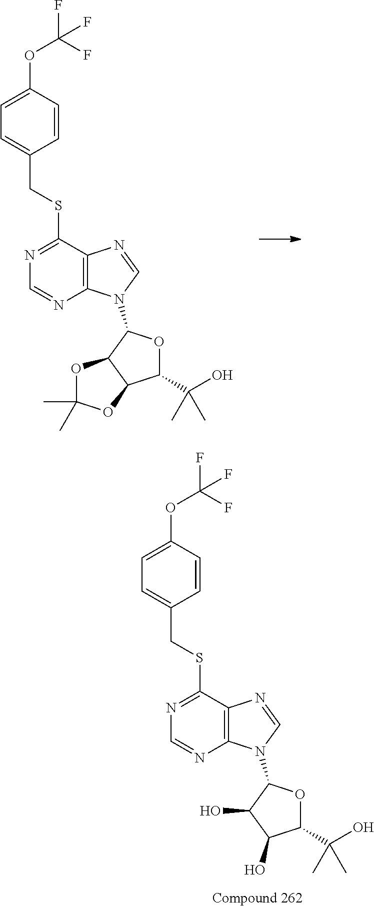

To a mixture of ((3aR,4R,6R,6aR)-6-(6-amino-9H-purin-9-yl)-2,2-dimethyltetrahydrofuro[3,4-d][1,3]dioxol-4-yl)methanol (4 g, 13.02 mmol, 1 eq) in water (13 mL) and MeCN (13 mL) was added [acetoxy(phenyl)-λ3-iodanyl]acetate (9.22 g, 28.64 mmol, 2.20 eq) and TEMPO (409.37 mg, 2.60 mmol, 0.2 eq). The mixture was stirred at 20° C. for 3 hr. The reaction mixture was then filtered and the filter cake was triturated with acetone (30 mL) to afford the corresponding acid compound (4.03 g, 96% yield) as a white solid.

LCMS: Rt=0.706 min; (ESI positive ion) m/z: 322.2 (M+H)+(calculated: 322.11).

1H NMR (400 MHz, DMSO-d6) δ=12.83 (br s, 1H), 8.24 (s, 1H), 8.08 (s, 1H), 7.28 (s, 2H), 6.33 (s, 1H), 5.54 (dd, J=1.8, 6.0 Hz, 1H), 5.47 (d, J=6.0 Hz, 1H), 4.69 (d, J=1.8 Hz, 1H), 1.52 (s, 3H), 1.35 (s, 3H).

To a mixture of the above acid compound (1 g, 3.11 mmol, 1 eq) in DCM (10 mL) and MeOH (10 mL) was added trimethylsilyldiazomethane (2 M, 4.98 mL, 3.2 eq) dropwise. The mixture was stirred at 20° C. for 12 hr. The reaction mixture was diluted with EtOAc (40 mL) and stirred at 20° C. for 30 min. Then the mixture was filtered and the filter cake was washed with EtOAc (20 mL×3) and dried to afford the ester compound (950 mg, crude) as a white solid.

LCMS: Rt=0.706 min; (ESI positive ion) m/z: 336.2 (M+H)+(calculated: 336.12).

To a solution of the above ester compound (950 mg, 2.83 mmol, 1 eq) in THF (20 mL) was added chloro(methyl)magnesium (3 M, 4.72 mL, 5 eq) dropwise at 0° C. under N2 atmosphere. Then the mixture was stirred at 20° C. for 1 hr. After stirred for 1 hr, the mixture was then stirred at 40° C. for 12 hr under nitrogen atmosphere. The reaction mixture was then poured into a saturated aqueous solution of NH4Cl (200 mL) at 0° C. and stirred for 5 min. The mixture was extracted with EtOAc (100 mL×3). The organic layer was washed with brine, dried by Na2SO4 and concentrated to give the residue. The residue was purified by Prep-HPLC (column: Waters Xbridge C18 150×50 mm×10 μm; mobile phase: [water(10 mM NH4HCO3)-ACN]; B %: 12%-42%, 11 min). The purified solution was lyophilized to afford the corresponding alcohol compound (428 mg, 45% yield) as a white solid.

LCMS: Rt=0.706 min; (ESI positive ion) m/z: 336.2 (M+H)+(calculated: 336.16).

1H NMR (400 MHz, DMSO-d6) δ=8.39 (s, 1H), 8.13 (s, 1H), 7.38 (s, 2H), 6.07 (d, J=4.2 Hz, 1H), 5.29 (s, 1H), 5.15 (dd, J=4.3, 6.2 Hz, 1H), 4.97 (dd, J=2.9, 6.4 Hz, 1H), 3.95 (d, J=2.9 Hz, 1H), 1.56 (s, 3H), 1.32 (s, 3H), 1.16 (s, 3H), 1.12 (s, 3H).

To a solution of the above alcohol compound (428 mg, 1.28 mmol, 1 eq) in DCM (5 mL) was added trimethylsilyl chloride (970.58 mg, 8.93 mmol, 1.13 mL, 7 eq) dropwise under nitrogen atmosphere at 20° C. After stirring for 15 min, tert-butyl nitrite (1.11 g, 10.72 mmol, 1.28 mL, 8.4 eq) was slowly added dropwise to the mixture. The mixture was stirred at 20° C. for 48 hr. The reaction mixture was then quenched by a saturated aqueous solution of NaHCO3 (100 mL) and extracted with DCM (40 mL×2). The organic layer was washed with brine, dried by Na2SO4 and concentrated to give a residue. The residue was purified by Prep-HPLC (column: Waters Xbridge C18 150×50 mm×10 μm; mobile phase: [water(10 mM NH4HCO3)-ACN]; B %: 23%-53%, 11 min). The purified solution was lyophilized to afford the corresponding chlorinated compound (122 mg, 27% yield) as a white solid.

LCMS: Rt=0.803 min; (ESI positive ion) m/z: 355.0 (M+H)+(calculated: 355.11).

A solution of the above chlorinated compound (119 mg, 335.41 μmol, 1 eq) and S-(4-(trifluoromethoxy)benzyl) ethanethioate (100.72 mg, 402.49 μmol, 1.2 eq) in MeOH (2 mL) and THE (0.5 mL) was stirred at 0° C. while K2CO3 (97.35 mg, 704.36 μmol, 2.1 eq) was added in one portion. The mixture was stirred at 0° C. for 2 hr. Water (10 mL) was then added to reaction mixture and the mixture was extracted with EtOAc (10 mL×3). The organic layer was washed with brine, dried by Na2SO4 and concentrated to afford the corresponding thioether compound (188 mg, crude) as a yellow oil.

LCMS: Rt=1.131 min; (ESI positive ion) m/z: 527.2 (M+H)+(calculated: 527.15).

To a mixture of the above thioether compound (183 mg, 347.56 μmol, 1 eq) in water (0.4 mL) was added formic acid (16.70 mg, 347.56 μmol, 1.6 mL, 1 eq) at 0° C. The mixture was stirred at 20° C. for 3 hr. The solvent was removed under reduced pressure to give a residue, which was purified by Prep-HPLC (column: Phenomenex Gemini-NX C18 75×30 mm×3 μm; mobile phase: [water(0.225% FA)-ACN]; B %: 38%-68%, 7 min). The purified solution was lyophilized to afford Compound 262 (58.52 mg, 35% yield) as an off-white solid.

LCMS: Rt=0.903 min; (ESI positive ion) m/z: 487.1 (M+H)+(calculated: 487.12).

1H NMR (400 MHz, DMSO-d6) δ=8.78 (s, 1H), 8.74 (s, 1H), 7.60 (d, J=8.7 Hz, 2H), 7.31 (d, J=8.1 Hz, 2H), 5.97 (d, J=6.7 Hz, 1H), 5.42 (d, J=6.4 Hz, 1H), 5.15 (d, J=4.6 Hz, 1H), 4.98 (s, 1H), 4.71 (s, 2H), 4.56-4.49 (m, 1H), 4.19 (dt, J=2.4, 4.9 Hz, 1H), 3.74 (d, J=2.3 Hz, 1H), 1.16 (s, 3H), 1.14 (s, 3H).

### Compound 263:

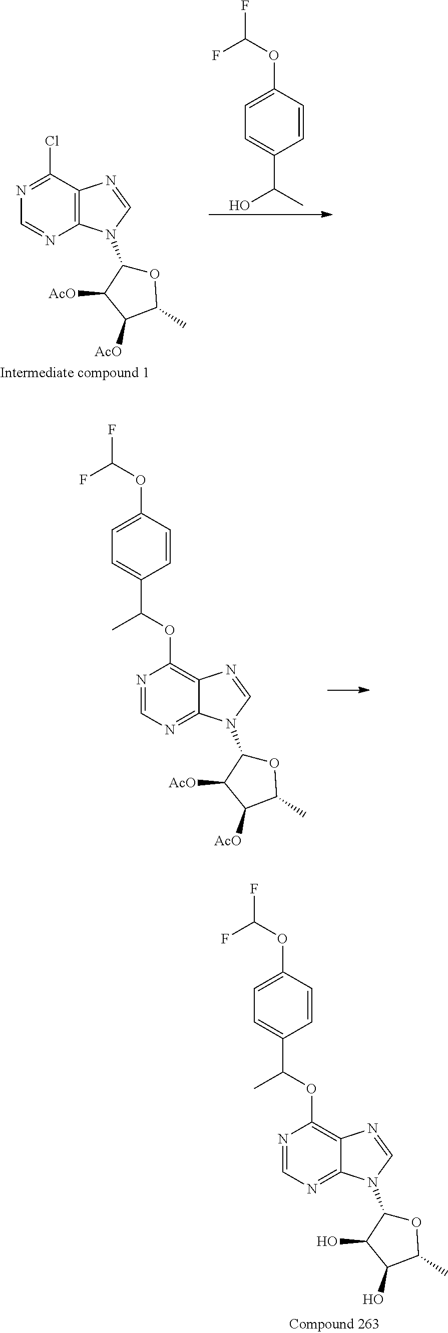 [SMILES: CC(=O)O[C@@H]1[C@H](OC(C)=O)[C@@H](C)O[C@H]1n1cnc2c(Cl)ncnc21 ; CC(O)c1ccc(OC(F)F)cc1 ; CC(=O)O[C@@H]1[C@H](OC(C)=O)[C@@H](C)O[C@H]1n1cnc2c(OC(C)c3ccc(OC(F)F)cc3)ncnc21 ; CC(Oc1ncnc2c1ncn2[C@@H]1O[C@H](C)[C@@H](O)[C@H]1O)c1ccc(OC(F)F)cc1]

Compound 263 (12.79 mg, 33% yield) was synthesized from Intermediate Compound 1 and the 1-(4-(difluoromethoxy)phenyl)ethan-1-ol following the protocols described for Compound 228.

LCMS: Rt=0.874 min; (ESI positive ion) m/z: 423.1 (M+H)+(calculated: 423.14).

1H NMR (400 MHz, DMSO-d6) δ=8.59 (s, 1H), 8.49 (d, J=1.0 Hz, 1H), 7.54 (dd, J=1.6, 8.6 Hz, 2H), 7.40-7.01 (m, 3H), 6.52 (q, J=6.6 Hz, 1H), 5.92 (dd, J=1.4, 5.0 Hz, 1H), 5.46 (d, J=5.6 Hz, 1H), 5.19 (d, J=4.2 Hz, 1H), 4.69 (quin, J=5.4 Hz, 1H), 4.04-3.93 (m, 2H), 1.71-1.66 (m, 3H), 1.31 (br d, J=5.2 Hz, 3H).

### Compound 265:

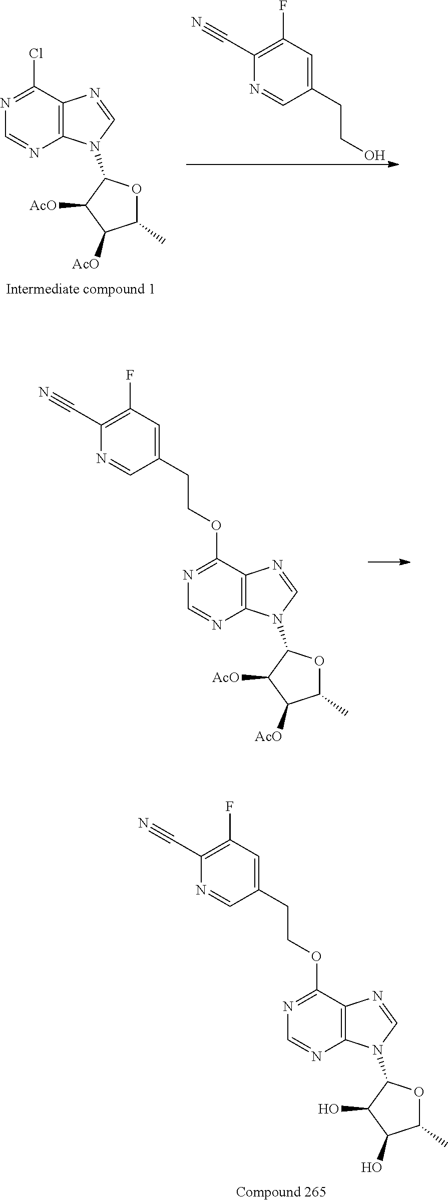 [SMILES: CC(=O)O[C@@H]1[C@H](OC(C)=O)[C@@H](C)O[C@H]1n1cnc2c(Cl)ncnc21 ; N#Cc1ncc(CCO)cc1F ; CC(=O)O[C@@H]1[C@H](OC(C)=O)[C@@H](C)O[C@H]1n1cnc2c(OCCc3cnc(C#N)c(F)c3)ncnc21 ; C[C@H]1O[C@@H](n2cnc3c(OCCc4cnc(C#N)c(F)c4)ncnc32)[C@H](O)[C@@H]1O]

Compound 265 (19.75 mg, 40% yield) was synthesized from Intermediate Compound 1 and 3-fluoro-5-(2-hydroxyethyl)picolinonitrile following the protocols described for Compound 188.

LCMS: Rt=0.767 min; (ESI positive ion) m/z: 401.2 (M+H)+(calculated: 401.13).

1H NMR (400 MHz, DMSO-d6) δ=8.65 (s, 1H), 8.58 (s, 1H), 8.54 (s, 1H), 8.16 (dd, J=1.3, 10.1 Hz, 1H), 5.93 (d, J=5.0 Hz, 1H), 5.50 (br d, J=4.9 Hz, 1H), 5.23 (br d, J=2.9 Hz, 1H), 4.86 (t, J=6.4 Hz, 2H), 4.67 (br d, J=4.0 Hz, 1H), 4.04-3.95 (m, 2H), 3.37-3.33 (m, 2H), 1.31 (d J=6.1 Hz, 3H).

### Compound 266:

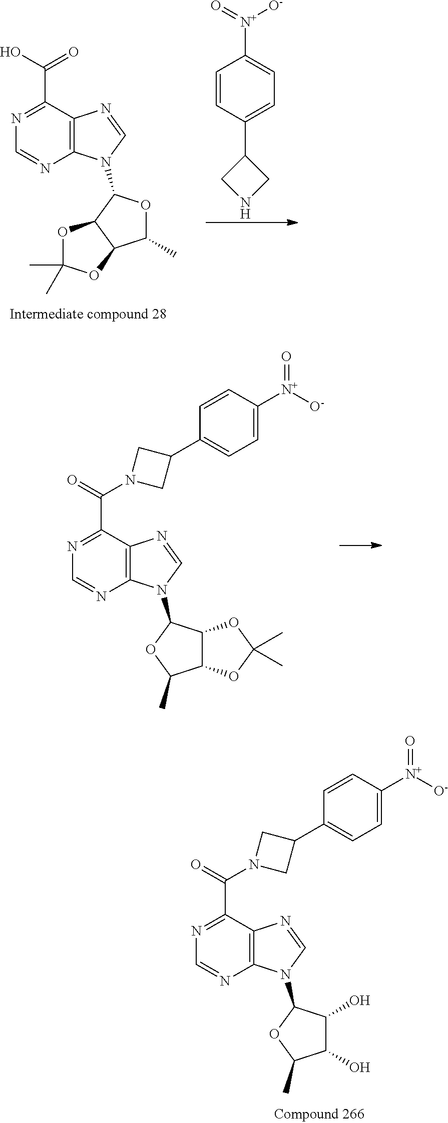 [SMILES: C[C@H]1O[C@@H](n2cnc3c(C(=O)O)ncnc32)[C@@H]2OC(C)(C)O[C@@H]21 ; O=[N+]([O-])c1ccc(C2CNC2)cc1 ; C[C@H]1O[C@@H](n2cnc3c(C(=O)N4CC(c5ccc([N+](=O)[O-])cc5)C4)ncnc32)[C@@H]2OC(C)(C)O[C@@H]21 ; C[C@H]1O[C@@H](n2cnc3c(C(=O)N4CC(c5ccc([N+](=O)[O-])cc5)C4)ncnc32)[C@H](O)[C@@H]1O]

To a mixture of Intermediate Compound 28 (82.27 mg, 256.86 μmol, 1 eq) and 3-(4-nitrophenyl)azetidine (90 mg, 256.86 μmol, 1 eq, TsOH) in THE (1.5 mL) was added propanephosphonic acid anhydride (204.32 mg, 642.14 μmol, 190.95 μL, 2.5 eq) and DIEA (165.99 mg, 1.28 mmol, 223.70 μL, 5 eq) at 0° C. The mixture was stirred at 25° C. for 1 hr. The reaction mixture was then concentrated under reduced pressure to give a residue, which was purified by Prep-TLC (SiO2, DCM:MeOH=10:1, Rf=0.5) to afford the corresponding amide compound (90 mg, 53% yield) as a white oil.

LCMS: Rt=0.805 min; (ESI positive ion) m/z: 481.2 (M+H)+(calculated: 481.18).

To a solution of the above amide compound (90 mg, 187.32 μmol, 1 eq) in water (0.1 mL) was added formic acid (488.00 mg, 10.60 mmol, 0.4 mL, 56.60 eq). The mixture was stirred at 0° C. for 7 hr. NH3·H2O (2 mL) was added to the reaction mixture at 0° C. until the pH was 7. The mixture was then filtered and concentrated under reduced pressure to give a residue. The residue was purified by Prep-HPLC (column: Waters Xbridge 150×25 mm×5 μm; mobile phase: [water (ammonia hydroxide v/v)-ACN]; B %: 13%-43%, 9 min). The purified solution was lyophilized to afford Compound 266 (10 mg, 12% yield) as a white solid.

LCMS: Rt=0.805 min; (ESI positive ion) m/z: 441.2 (M+H)+(calculated: 441.14).

1H NMR (400 MHz, DMSO-d6) δ=9.08-9.00 (m, 1H), 8.94 (s, 1H), 8.24 (d, J=8.4 Hz, 2H), 7.71 (d, J=8.7 Hz, 2H), 6.02 (d, J=5.0 Hz, 1H), 5.74-5.19 (m, 2H), 4.74 (br t, J=4.7 Hz, 1H), 4.70-4.60 (m, 2H), 4.34 (br dd, J=5.9, 8.7 Hz, 1H), 4.23-4.14 (m, 2H), 4.06-3.99 (m, 2H), 1.34 (br d, J=5.9 Hz, 3H).

### Compound 267:

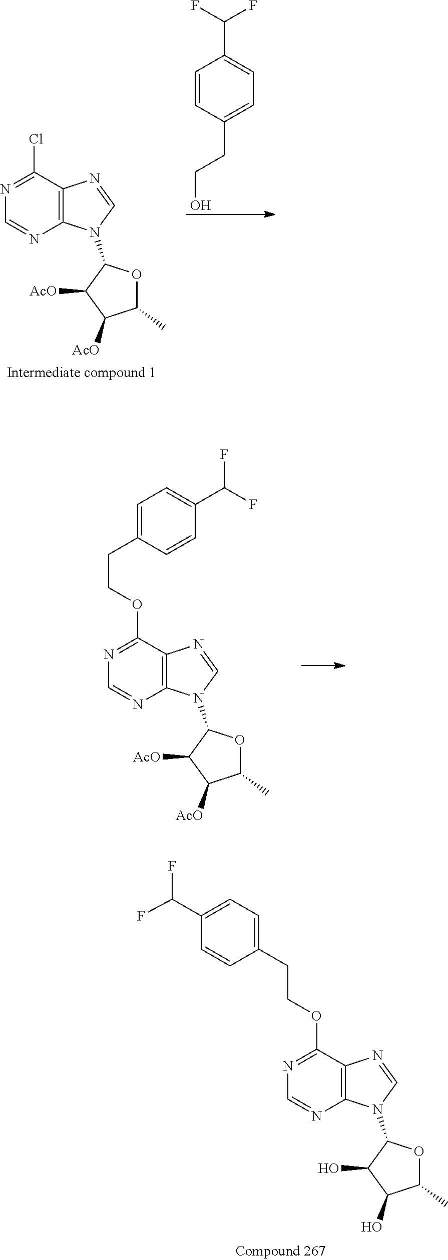 [SMILES: CC(=O)O[C@@H]1[C@H](OC(C)=O)[C@@H](C)O[C@H]1n1cnc2c(Cl)ncnc21 ; OCCc1ccc(C(F)F)cc1 ; CC(=O)O[C@@H]1[C@H](OC(C)=O)[C@@H](C)O[C@H]1n1cnc2c(OCCc3ccc(C(F)F)cc3)ncnc21 ; C[C@H]1O[C@@H](n2cnc3c(OCCc4ccc(C(F)F)cc4)ncnc32)[C@H](O)[C@@H]1O]

Compound 267 (16.75 mg, 14% yield) was synthesized from Intermediate Compound 1 and 2-(4-(difluoromethyl)phenyl)ethan-1-ol following the protocols described for Compound 188.

LCMS: Rt=0.841 min; (ESI positive ion) m/z: 406.9 (M+H)+(calculated: 407.15).

1H NMR (400 MHz, DMSO-d6) δ=8.55 (d, J=6.8 Hz, 2H), 7.53-7.47 (m, 4H), 7.15-6.84 (m, 1H), 5.93 (d, J=4.9 Hz, 1H), 5.48 (d, J=5.6 Hz, 1H), 5.22 (br d, J=4.8 Hz, 1H), 4.79 (t, J=6.7 Hz, 2H), 4.67 (q, J=5.1 Hz, 1H), 4.02-3.96 (m, 2H), 3.21 (t, J=6.7 Hz, 2H), 1.31 (d, J=6.1 Hz, 3H).

### Compound 268:

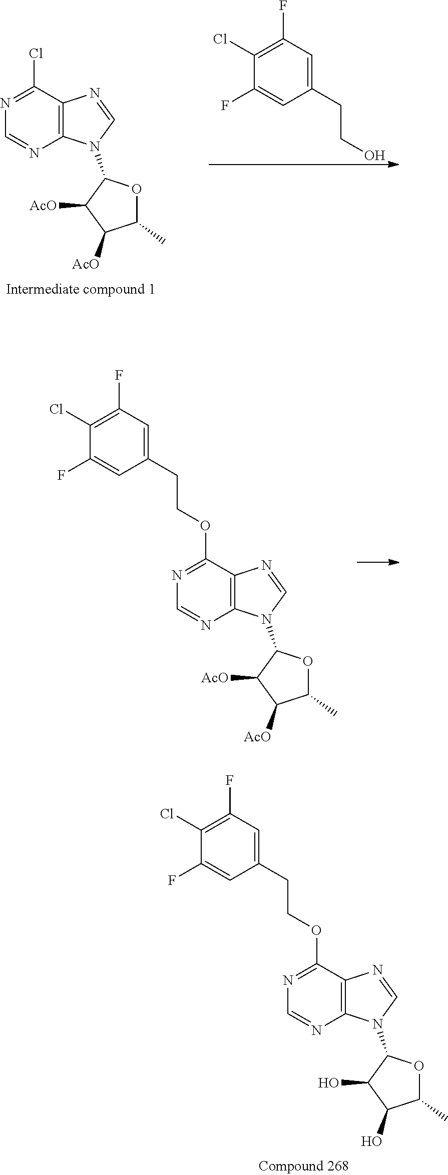 [SMILES: CC(=O)O[C@@H]1[C@H](OC(C)=O)[C@@H](C)O[C@H]1n1cnc2c(Cl)ncnc21 ; OCCc1cc(F)c(Cl)c(F)c1 ; CC(=O)O[C@@H]1[C@H](OC(C)=O)[C@@H](C)O[C@H]1n1cnc2c(OCCc3cc(F)c(Cl)c(F)c3)ncnc21 ; C[C@H]1O[C@@H](n2cnc3c(OCCc4cc(F)c(Cl)c(F)c4)ncnc32)[C@H](O)[C@@H]1O]

Compound 268 (4.7 mg, 4% yield) was synthesized from Intermediate Compound 1 and 2-(4-chloro-3,5-difluorophenyl)ethan-1-ol following the protocols described for Compound 188.

LCMS: Rt=0.871 min; (ESI positive ion) m/z: 426.9 (M+H)+(calculated: 427.09).

1H NMR (400 MHz, DMSO-d6) δ=8.56 (d, J=12.2 Hz, 2H), 7.36 (br d, J=8.4 Hz, 2H), 5.93 (d, J=4.8 Hz, 1H), 5.48 (br d, J=5.4 Hz, 1H), 5.21 (br d, J=3.8 Hz, 1H), 4.79 (br t, J=6.4 Hz, 2H), 4.71-4.63 (m, 1H), 3.99 (br d, J=4.2 Hz, 2H), 3.19 (br t, J=6.4 Hz, 2H), 1.31 (br d, J=6.0 Hz, 3H).

### Compound 271:

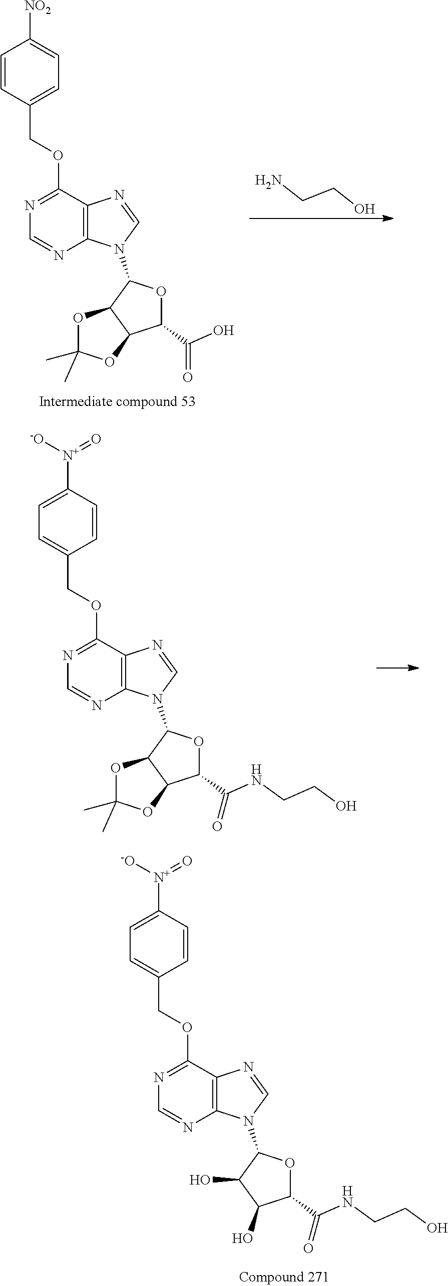

To a solution of Intermediate Compound 53 (80 mg, 174.90 μmol, 1 eq) and 2-aminoethanol (32.05 mg, 524.71 μmol, 31.73 μL, 3 eq) in THE (3 mL) was added DIEA (113.03 mg, 874.52 μmol, 152.33 μL, 5 eq) and propanephosphonic acid anhydride (333.91 mg, 524.71 mol, 312.06 μL, 50% purity, 3 eq) at 0° C. The mixture was stirred at 25° C. for 1 hour. The reaction mixture was filtered and concentrated under reduced pressure to give a residue, which was purified by column chromatography (SiO2, Petroleum ether:Ethyl acetate=1:0 to 1:1) to afford the corresponding amide compound (70 mg, 80% yield) as a yellow oil.

LCMS: Rt=0.848 min; (ESI positive ion) m/z: 501.2 (M+H)+(calculated: 501.17).

To a solution of the above amide compound (50 mg, 99.91 μmol, 1 eq) in water (0.1 mL) was added formic acid (4.80 mg, 99.91 μmol, 0.4 mL, 1 eq) at 0° C. The mixture was stirred at 25° C. for 1 hour. NH3·H2O (0.1 ml) and MeOH (0.5 ml) were added and the mixture was stirred at 25° C. for 10 min. The reaction mixture was filtered and concentrated under reduced pressure to give a residue. The residue was purified by Prep-HPLC (column: Waters Xbridge 150×25 mm×5 μm; mobile phase: [water (ammonia hydroxide v/v)-ACN]; B %: 12%-40%, 9 min) to afford Compound 271 (27 mg, 58% yield) as a white solid.

LCMS: Rt=0.762 min; (ESI positive ion) m/z: 461.2 (M+H)+(calculated: 461.13).

1H NMR (400 MHz, MeOD-d4) δ=8.60 (d, J=17.2 Hz, 2H), 8.27 (br d, J=8.7 Hz, 2H), 7.80 (br d, J=8.6 Hz, 2H), 6.14 (d, J=7.5 Hz, 1H), 5.84 (s, 2H), 4.82-4.80 (m, 2H), 4.40 (dd, J=1.5, 4.7 Hz, 1H), 3.66 (dt, J=3.4, 5.4 Hz, 2H), 3.49-3.41 (m, 2H).

### Compound 276:

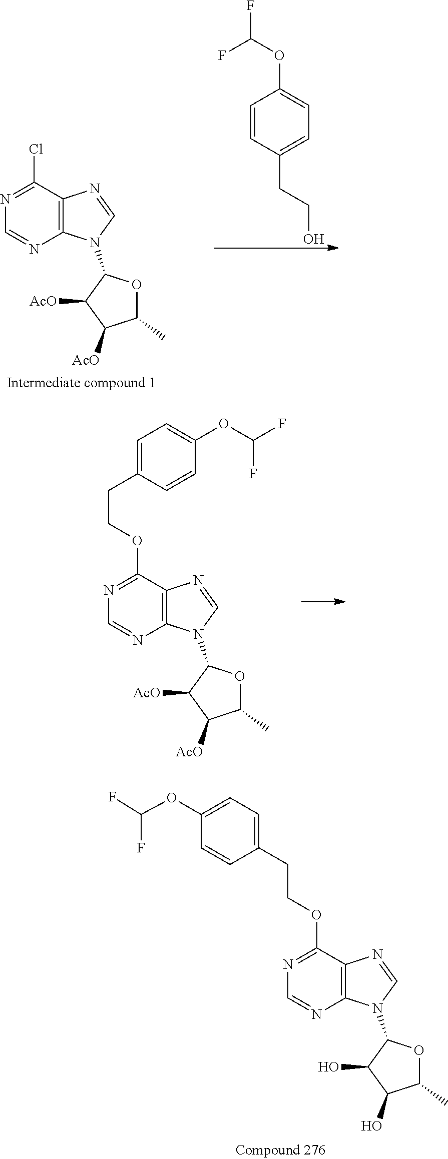 [SMILES: CC(=O)O[C@@H]1[C@H](OC(C)=O)[C@@H](C)O[C@H]1n1cnc2c(Cl)ncnc21 ; OCCc1ccc(OC(F)F)cc1 ; CC(=O)O[C@@H]1[C@H](OC(C)=O)[C@@H](C)O[C@H]1n1cnc2c(OCCc3ccc(OC(F)F)cc3)ncnc21 ; C[C@H]1O[C@@H](n2cnc3c(OCCc4ccc(OC(F)F)cc4)ncnc32)[C@H](O)[C@@H]1O]

Compound 276 (42.03 mg, 20% yield) as synthesized from Intermediate Compound 1 and 2-(4-(difluoromethoxy)phenyl)ethan-1-ol following the protocols described for Compound 188.

LCMS: Rt=0.864 min; (ESI positive ion) m/z: 445.3 (M+Na)+(calculated: 445.13).

1H NMR (400 MHz, DMSO-d6) δ=8.55 (d, J=10.8 Hz, 2H), 7.39 (d, J=8.5 Hz, 2H), 7.38-7.00 (t, 1H), 7.12 (d, J=8.4 Hz, 2H), 5.93 (d, J=5.0 Hz, 1H), 5.48 (d, J=5.6 Hz, 1H), 5.21 (d, J=4.9 Hz, 1H), 4.76 (t, J=6.8 Hz, 2H), 4.68 (q, J=5.1 Hz, 1H), 4.05-3.95 (m, 2H), 3.14 (t, J=6.8 Hz, 2H), 1.31 (d, J=6.1 Hz, 3H)

### Compound 278:

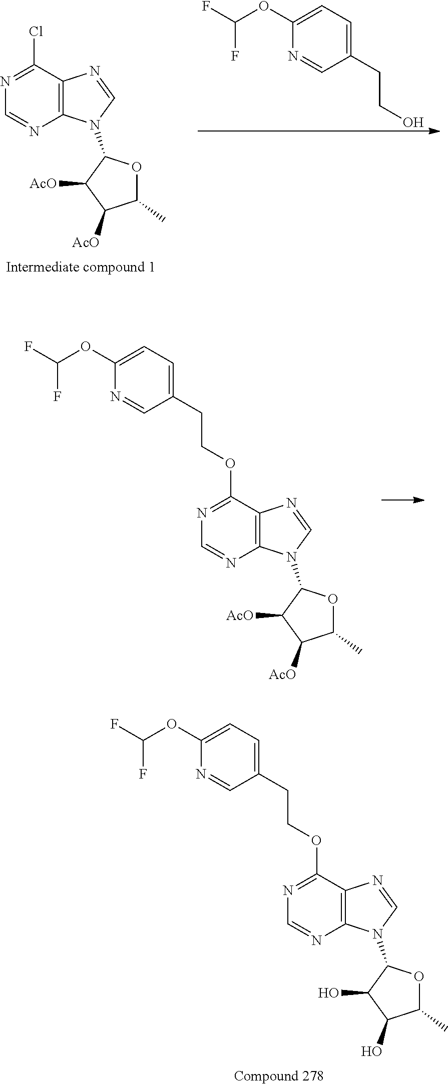 [SMILES: CC(=O)O[C@@H]1[C@H](OC(C)=O)[C@@H](C)O[C@H]1n1cnc2c(Cl)ncnc21 ; OCCc1ccc(OC(F)F)nc1 ; CC(=O)O[C@@H]1[C@H](OC(C)=O)[C@@H](C)O[C@H]1n1cnc2c(OCCc3ccc(OC(F)F)nc3)ncnc21 ; C[C@H]1O[C@@H](n2cnc3c(OCCc4ccc(OC(F)F)nc4)ncnc32)[C@H](O)[C@@H]1O]

Compound 278 (42.03 mg, 20% yield) was synthesized from Intermediate Compound 1 and 2-(6-(difluoromethoxy)pyridin-3-yl)ethan-1-ol following the protocols described for Compound 188.

LCMS: Rt=0.831 min; (ESI positive ion) m/z: 424.3 (M+Na)+(calculated: 424.14).

1H NMR (400 MHz, DMSO-d6) δ=8.57 (s, 1H), 8.54 (s, 1H), 8.22 (d, J=1.7 Hz, 1H), 7.92 (dd, J=2.1, 8.3 Hz, 1H), 7.67 (t, J=72.8 Hz, 1H), 7.05 (d, J=8.3 Hz, 1H), 5.93 (d, J=4.9 Hz, 1H), 5.50 (br d, J=4.4 Hz, 1H), 5.23 (br s, 1H), 4.77 (t, J=6.5 Hz, 2H), 4.68 (br d, J=3.9 Hz, 1H), 3.99 (br d, J=4.4 Hz, 2H), 3.16 (t, J=6.5 Hz, 2H), 1.31 (d, J=5.9 Hz, 3H).

### Compound 279:

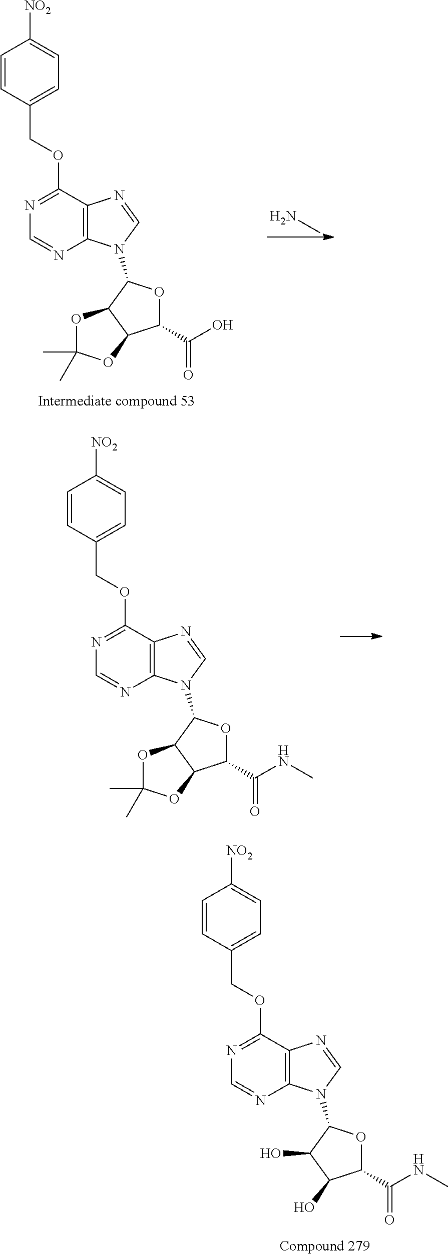

Compound 279 (9 mg, 16% yield) was synthesized from Intermediate Compound 53 and methylamine following the protocols described for Compound 271.

LCMS: Rt=0.798 min; (ESI positive ion) m/z: 430.9 (M+H)+(calculated: 431.12).

1H NMR (400 MHz, MeOD-d4) δ=8.60 (d, J=9.3 Hz, 2H), 8.27 (d, J=8.6 Hz, 2H), 7.80 (d, J=8.6 Hz, 2H), 6.14 (d, J=7.3 Hz, 1H), 5.84 (s, 2H), 4.81-4.78 (m, 1H), 4.50 (s, 1H), 4.41-4.36 (m, 1H), 2.86 (s, 3H).

### Compound 280:

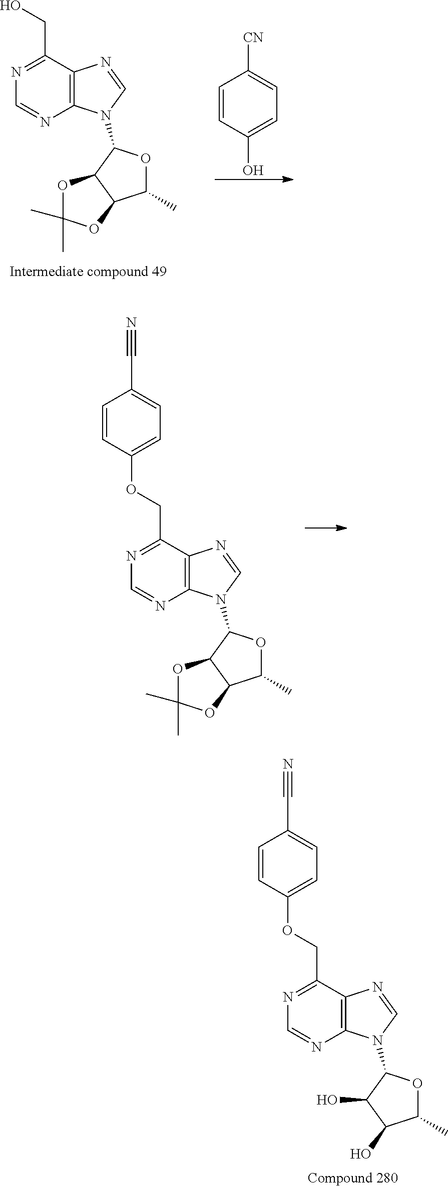 [SMILES: C[C@H]1O[C@@H](n2cnc3c(CO)ncnc32)[C@@H]2OC(C)(C)O[C@@H]21 ; N#Cc1ccc(O)cc1 ; C[C@H]1O[C@@H](n2cnc3c(COc4ccc(C#N)cc4)ncnc32)[C@@H]2OC(C)(C)O[C@@H]21 ; C[C@H]1O[C@@H](n2cnc3c(COc4ccc(C#N)cc4)ncnc32)[C@H](O)[C@@H]1O]

Compound 280 (9.15 mg, 51% yield) was synthesized from Intermediate Compound 49 and 4-hydroxybenzonitrile following the protocols described for Compound 233.

LCMS: Rt=0.753 min; (ESI positive ion) m/z: 367.9 (M+H)+(calculated: 368.13).

1H NMR (400 MHz, DMSO-d6) δ=8.95 (s, 1H), 8.84 (s, 1H), 7.76 (d, J=8.4 Hz, 2H), 7.22 (d, J=8.4 Hz, 2H), 6.00 (d, J=5.0 Hz, 1H), 5.68 (s, 2H), 5.52 (d, J=5.6 Hz, 1H), 5.24 (br d, J=5.0 Hz, 1H), 4.78-4.71 (m, 1H), 4.02 (br d, J=4.4 Hz, 2H), 1.33 (d, J=5.8 Hz, 3H).

### Compound 283:

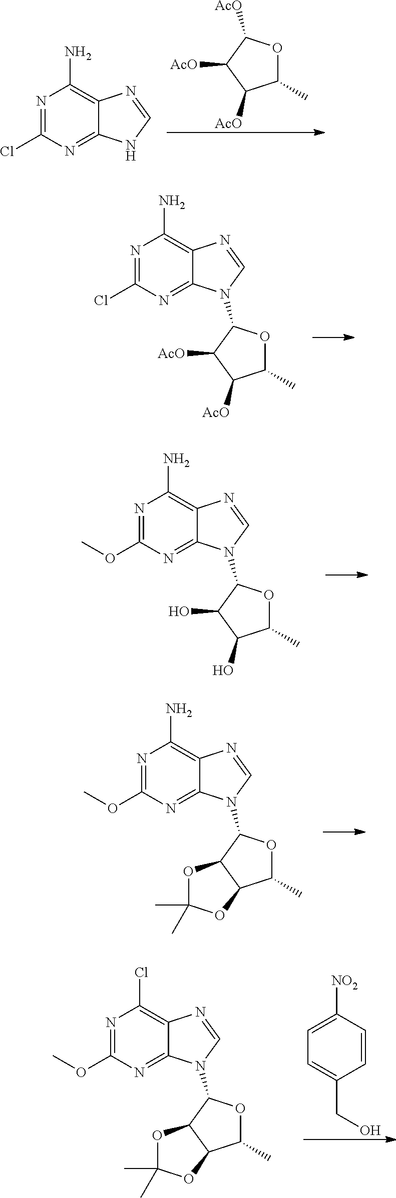

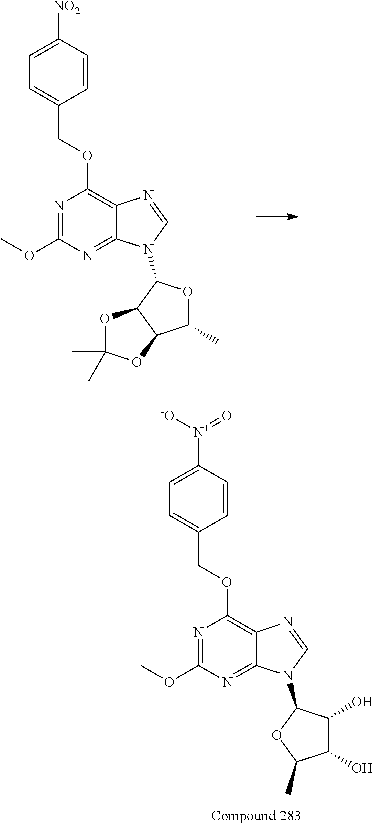

To a solution of (2S,3R,4R,5R)-5-methyltetrahydrofuran-2,3,4-triyl triacetate (8.44 g, 32.43 mmol, 1 eq) and 2-chloro-9H-purin-6-amine (6.6 g, 38.92 mmol, 1.2 eq) in 1,2-dichloroethane (100 mL) was added bis(trimethylsilyl)acetamide (13.20 g, 64.87 mmol, 16.03 mL, 2 eq) at 0° C. The mixture was stirred at 80° C. for 3 hr until the solution became clear. After the solution became clear, the mixture was cooled to 0° C. and trimethylsilyl trifluoromethanesulfonate (9.37 g, 42.17 mmol, 7.62 mL, 1.3 eq) was added. The mixture was then stirred at 80° C. for 12 hr. The reaction mixture was poured into a saturated aqueous solution of NaHCO3 (50 mL) and extracted with DCM (50 mL×3). The organic layer was washed with brine, dried by Na2SO4 and concentrated to give a residue. The residue was purified by silica gel chromatography (column height: 250 mm, diameter: 100 mm, 100-200 mesh silica gel, Petroleum ether/Ethyl acetate=1/1) to afford the conjugated intermediate compound (8 g, 64% yield) as a white solid.

LCMS: Rt=0.797 min; (ESI positive ion) m/z: 369.9 (M+H)+(calculated: 370.08).

To a mixture of the above conjugated intermediate compound (3 g, 8.11 mmol, 1 eq) in MeOH (30 mL) was added sodium methoxide (5 M, 8.11 mL, 5 eq) at 0° C. under N2. The mixture was stirred at 50° C. for 16 hr under N2 atmosphere and then filtered and concentrated under reduced pressure to give a residue. The residue was purified by Prep-HPLC (0.1% FA condition) to afford the corresponding ether compound (1.1 g, 47% yield) as a white solid.

LCMS: Rt=0.318 min; (ESI positive ion) m/z: 282.1 (M+H)+(calculated: 282.11).

1H NMR (400 MHz, DMSO-d6) δ=8.30 (s, 1H), 8.15-8.10 (m, 1H), 7.29 (br s, 2H), 5.74 (d, J=4.9 Hz, 1H), 4.66 (t, J=5.1 Hz, 1H), 4.03-3.98 (m, 1H), 3.98-3.90 (m, 1H), 3.80 (s, 3H), 1.29 (d, J=6.2 Hz, 3H).

To a solution of the above ether compound (1.1 g, 3.91 mmol, 1 eq) in acetone (30 mL) was added p-toluenesulfonic acid monohydrate (7.44 g, 39.11 mmol, 10 eq). The mixture was stirred at 20° C. for 3 hr. The NaHCO3 was added to the reaction mixture until the pH reached about 7. The mixture was then extracted with DCM (40 mL×3). The combined organic phase was washed with brine (80 mL), dried with anhydrous Na2SO4, filtered and concentrated under vacuum. The resultant residue was purified by column chromatography (SiO2, Petroleum ether/Ethyl acetate=5/1 to 0/1) to afford the corresponding acetonide compound (700 mg, 54% yield) as a white solid.

LCMS: Rt=0.734 min; (ESI positive ion) m/z: 322.0 (M+H)+(calculated: 322.14).

1H NMR (400 MHz, DMSO-d6) δ=8.12 (s, 1H), 7.34 (br s, 2H), 6.02 (d, J=2.3 Hz, 1H), 5.48 (dd, J=2.3, 6.2 Hz, 1H), 4.81 (dd, J=3.5, 6.2 Hz, 1H), 4.21 (dq, J=3.7, 6.5 Hz, 1H), 3.82 (s, 3H), 1.52 (s, 3H), 1.31 (s, 3H), 1.26 (d, J=6.6 Hz, 3H).

To a solution of the above acetonide compound (400 mg, 1.24 mmol, 1 eq) in DCM (10 mL) was added trimethylsilyl chloride (1.22 g, 11.20 mmol, 1.42 mL, 9 eq) and tert-butyl nitrite (770.20 mg, 7.47 mmol, 888.35 μL, 6 eq) at 0° C. The mixture was stirred at 20° C. for 12 hr. The reaction mixture was then diluted with water (20 mL) and extracted with DCM (20 mL×2). The combined organic layers were washed with brine (50 mL), dried over Na2SO4, filtered and concentrated under reduced pressure to give a residue. The residue was purified by column chromatography (SiO2, Petroleum ether/Ethyl acetate=5/1 to 0/1) to afford the corresponding chlorinated compound (120 mg, 28% yield) as a yellow gum.

LCMS: Rt=0.846 min; (ESI positive ion) m/z: 340.9 (M+H)+(calculated: 341.09).

To a mixture of the above chlorinated compound (100 mg, 293.46 μmol, 1 eq) and (4-nitrophenyl)methanol (44.94 mg, 293.46 μmol, 1 eq) in toluene (3 mL) was added Cs2CO3 (239.04 mg, 733.65 μmol, 2.5 eq), Xantphos (33.96 mg, 58.69 μmol, 0.2 eq), and Pd2(dba)3 (26.87 mg, 29.35 μmol, 0.1 eq) under N2. The mixture was degassed and purged with N2 3 times and stirred at 100° C. for 0.5 hr under N2 atmosphere. The reaction mixture was filtered and concentrated under reduced pressure to give a residue, which was purified by Prep-TLC (SiO2, Petroleum ether:Ethyl acetate=1:1, Rf=0.2) to afford the corresponding ether compound (110 mg, 78% yield) as a yellow oil.

LCMS: Rt=0.870 min; (ESI positive ion) m/z: 458.2 (M+H)+(calculated: 458.16).

To a solution of the above ether compound (100 mg, 218.61 μmol, 1 eq) in water (0.2 mL) was added formic acid (976.00 mg, 21.21 mmol, 0.8 mL, 97.00 eq) at 0° C. The mixture was stirred at 0° C. for 7 hr. NH3·H2O (2 mL) was added to the reaction mixture at 0° C. until the pH was 7. The mixture was then filtered and concentrated under reduced pressure to give a residue. The residue was purified by Prep-HPLC (column: Waters Xbridge 150×25 mm×5 μm; mobile phase: [water (ammonia hydroxide v/v)-ACN]; B %: 18%-45%, 9 min) and lyophilized to afford Compound 283 (50 mg, 55% yield) as a white solid.

LCMS: Rt=0.725 min; (ESI positive ion) m/z: 418.1 (M+H)+(calculated: 418.13).

1H NMR (400 MHz, DMSO-d6) δ=8.40 (s, 1H), 8.27 (d, J=8.7 Hz, 2H), 7.75 (d, J=8.7 Hz, 2H), 5.83 (d, J=5.0 Hz, 1H), 5.74 (s, 2H), 5.44 (d, J=5.3 Hz, 1H), 5.20 (d, J=5.0 Hz, 1H), 4.69 (q, J=5.2 Hz, 1H), 4.04-3.95 (m, 2H), 3.91 (s, 3H), 1.31 (d, J=6.2 Hz, 3H).

### Compound 284:

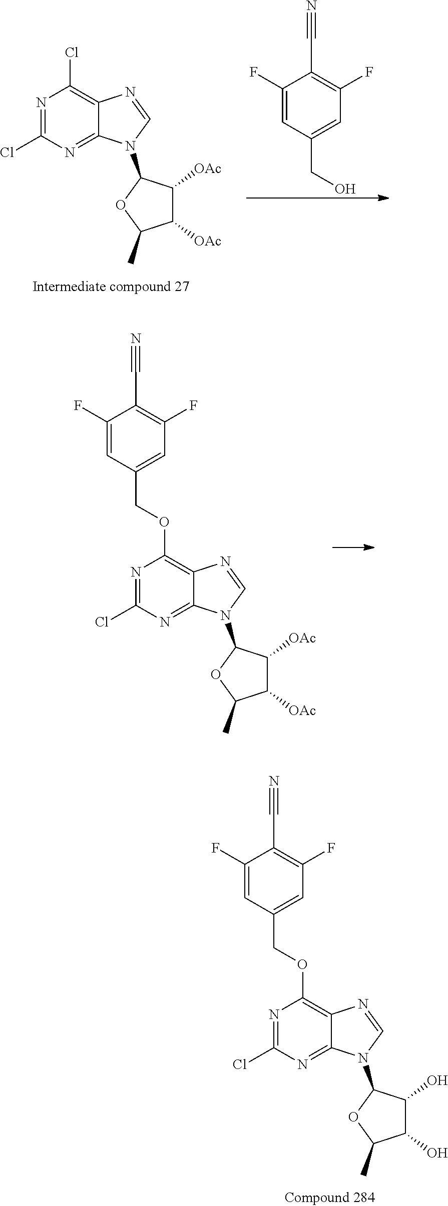 [SMILES: CC(=O)O[C@@H]1[C@H](OC(C)=O)[C@@H](C)O[C@H]1n1cnc2c(Cl)nc(Cl)nc21 ; N#Cc1c(F)cc(CO)cc1F ; CC(=O)O[C@@H]1[C@H](OC(C)=O)[C@@H](C)O[C@H]1n1cnc2c(OCc3cc(F)c(C#N)c(F)c3)nc(Cl)nc21 ; C[C@H]1O[C@@H](n2cnc3c(OCc4cc(F)c(C#N)c(F)c4)nc(Cl)nc32)[C@H](O)[C@@H]1O]

To a solution of Intermediate Compound 27 (120 mg, 308.33 μmol, 1 eq), 2,6-difluoro-4-(hydroxymethyl)benzonitrile (57.36 mg, 339.17 μmol, 1.1 eq), Cs2CO3 (251.15 mg, 770.83 μmol, 2.5 eq), and Xantphos (35.68 mg, 61.67 μmol, 0.2 eq) in toluene (5 mL) was added Pd2(dba)3 (28.23 mg, 30.83 μmol, 0.1 eq). The mixture was stirred at 80° C. for 1 hr. The reaction mixture was then concentrated under reduced pressure to give a residue, which was purified by Prep-TLC (SiO2, Petroleum ether:Ethyl acetate=1:1, Rf=0.35) to afford the corresponding ether compound (150 mg, 87% yield) as a yellow oil.

LCMS: Rt=0.471 min; (ESI positive ion) m/z: 522.1 (M+H)+(calculated: 522.09).

A mixture of the above ether compound (150 mg, 287.43 μmol, 1 eq) and NH3·H2O (1.82 g, 14.54 mmol, 2 mL, 28% purity, 50.59 eq) in MeOH (2 mL) was degassed and purged with N2 3 times. The mixture was stirred at 25° C. for 0.5 hr under N2 atmosphere. The reaction mixture was then concentrated under reduced pressure to give a residue, which was purified by Prep-HPLC (column: Phenomenex luna C18 150×25 mm×10 μm; mobile phase: [water(FA)-ACN]; B %: 33%-63%, 9 min) and lyophilized to give Compound 284 (44 mg, 35% yield) as a white solid.

LCMS: Rt=0.496 min; (ESI positive ion) m/z: 438.1 (M+H)+(calculated: 438.07).

1H NMR (400 MHz, DMSO-d6) δ=8.68 (s, 1H), 7.59 (d, J=8.8 Hz, 2H), 5.90 (d, J=5.0 Hz, 1H), 5.73 (s, 2H), 5.53 (d, J=5.8 Hz, 1H), 5.26 (d, J=5.4 Hz, 1H), 4.68-4.56 (m, 1H), 4.09-3.92 (m, 2H), 1.33 (d, J=6.4 Hz, 3H).

### Compound 285:

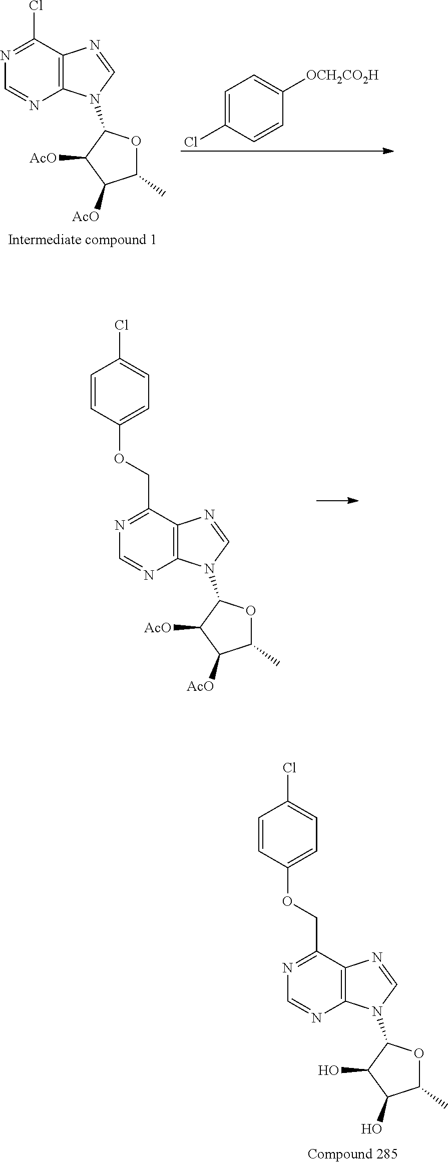

A mixture of Intermediate Compound 1 (50 mg, 140.95 μmol, 1 eq), 2-(4-chlorophenoxy)acetic acid (34.19 mg, 183.23 μmol, 1.3 eq), [4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate (1.58 mg, 1.41 μmol, 0.01 eq), bis(1,1-dimethylethyl)-2,2′-bipyridine]nickel (II) dichloride (2.80 mg, 7.05 μmol, 0.05 eq), and Cs2CO3 (68.88 mg, 211.42 μmol, 1.5 eq) in DMA (5 mL) was degassed and purged with N2 3 times. The mixture was then irradiated with two 34 W blue LED lamps (at approximately 7 cm away from the light source) to keep the reaction temperature at 25° C. overnight. The mixture was poured into water (200 mL) and extracted with EtOAc (100 mL×2). The combined organic was washed with brine (200 mL), dried with anhydrous Na2SO4, filtered and concentrated under vacuum to afford the corresponding ether compound (45 mg, crude) as a yellow oil.

To a solution of the above ether compound (40 mg, 86.79 μmol, 1 eq) in MeOH (1 mL) was added NH3·H2O (910.00 mg, 7.27 mmol, 1 mL, 28% purity, 83.77 eq). The mixture was stirred at 20° C. for 0.5 hr and then concentrated under vacuum. The residue was purified by Prep-HPLC (column: Unisil 3-100 C18 Ultra 150×50 mm×3 μm; mobile phase:[water(0.225% FA)-ACN]; B %:35%-65%, 10 min) to afford Compound 285 (12.13 mg, 36% yield) as a white solid.

LCMS: Rt=0.800 min; (ESI positive ion) m/z: 376.8 (M+H)+(calculated: 377.09).

1H NMR (400 MHz, DMSO-d6) δ=8.95 (s, 1H), 8.83 (s, 1H), 7.34-7.29 (m, 2H), 7.09-7.05 (m, 2H), 6.00 (d, J=5.0 Hz, 1H), 5.55 (s, 2H), 5.52 (d, J=5.6 Hz, 1H), 5.23 (d, J=4.8 Hz, 1H), 4.75 (q, J=5.0 Hz, 1H), 4.06-3.99 (m, 2H), 1.33 (d, J=6.2 Hz, 3H).

### Compound 287:

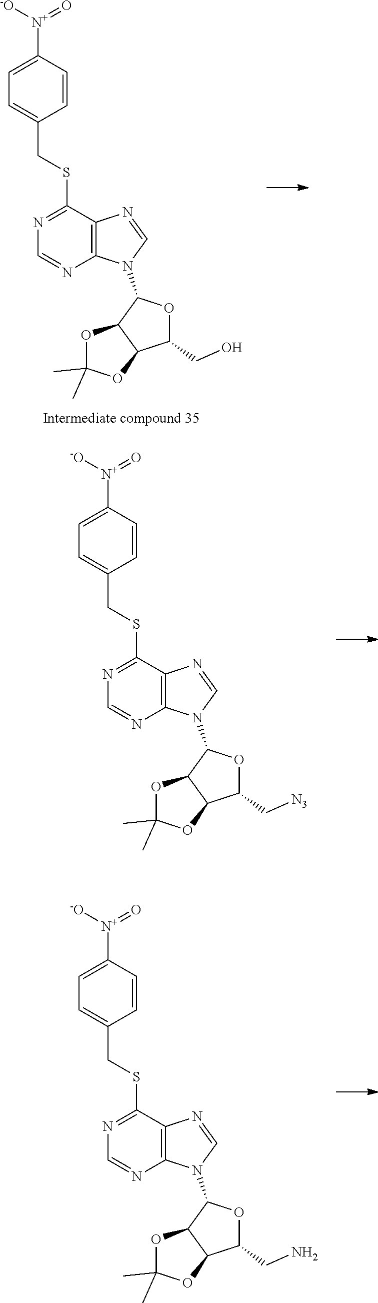

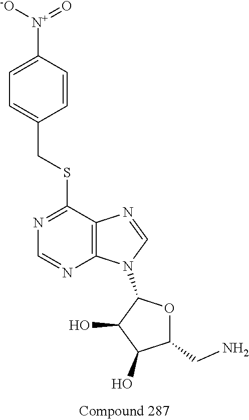

A mixture of Intermediate Compound 35 (100 mg, 217.64 μmol, 1 eq) and triphenylphosphine (79.92 mg, 304.70 μmol, 1.4 eq) in THE (1 mL) was cooled at 0° C. in an ice bath. DIAD (61.61 mg, 304.70 μmol, 59.24 μL, 1.4 eq) was added dropwise, followed by a solution of diphenylphosphoryl azide (83.85 mg, 304.70 μmol, 66.03 μL, 1.4 eq) in THE (1 mL). After 30 minutes, the reaction mixture was allowed to warm to 20° C. and stirred for 16 hours. The reaction mixture was filtered and concentrated under reduced pressure to give a residue, which was purified by Prep-TLC (SiO2, Petroleum ether:Ethyl acetate=1:1) to afford the corresponding azide compound (50 mg, 44% yield) as a colorless oil.

LCMS: Rt=0.977 min; (ESI positive ion) m/z: 484.9 (M+H)+(calculated: 485.13).

A mixture of the above azide compound (30 mg, 61.92 μmol, 1 eq) and triphenylphosphine (24.36 mg, 92.88 μmol, 1.5 eq) in THF (1 mL), H2O (0.2 mL) was stirred at 50° C. for 1 hour. The reaction mixture was filtered and concentrated under reduced pressure to give a residue, which was purified by Prep-HPLC (column: Waters Xbridge 150×25 mm×5 μm; mobile phase: [water (0.05% ammonia hydroxide v/v)-ACN]; B %: 30%-60%, 8 min) to afford the corresponding amine (15 mg, 53% yield) as a white solid.

LCMS: Rt=0.991 min; (ESI positive ion) m/z: 459.2 (M+H)+(calculated: 459.14).

To a solution of the above amine compound (15 mg, 32.72 μmol, 1 eq) in water (0.1 mL) was added TFA (616.00 mg, 5.40 mmol, 0.4 mL, 165.13 eq) at 0° C. The mixture was stirred at 20° C. for 2 hours. The reaction mixture was filtered and concentrated under reduced pressure to give a residue, which was purified by Prep-HPLC (column: Unisil 3-100 C18 Ultra 150×50 mm×3 μm; mobile phase: [water(0.225% FA)-ACN]; B %: 50%-35%, 10 min) to afford Compound 287 (10.6 mg, 77% yield) as a white solid.

LCMS: Rt=0.715 min; (ESI positive ion) m/z: 419.0 (M+H)+(calculated: 419.11).

1H NMR (400 MHz, MeOD-d4) δ=8.75 (s, 1H), 8.48 (s, 1H), 8.16 (d, J=8.8 Hz, 2H), 7.75 (d, J=8.8 Hz, 2H), 6.09 (d, J=5.0 Hz, 1H), 4.65-4.53 (m, 2H), 4.45 (t, J=4.7 Hz, 1H), 4.31-4.26 (m, 1H), 3.52 (dd, J=9.7, 13.1 Hz, 2H), 3.36 (br d, J=3.1 Hz, 1H).

### Compound 288:

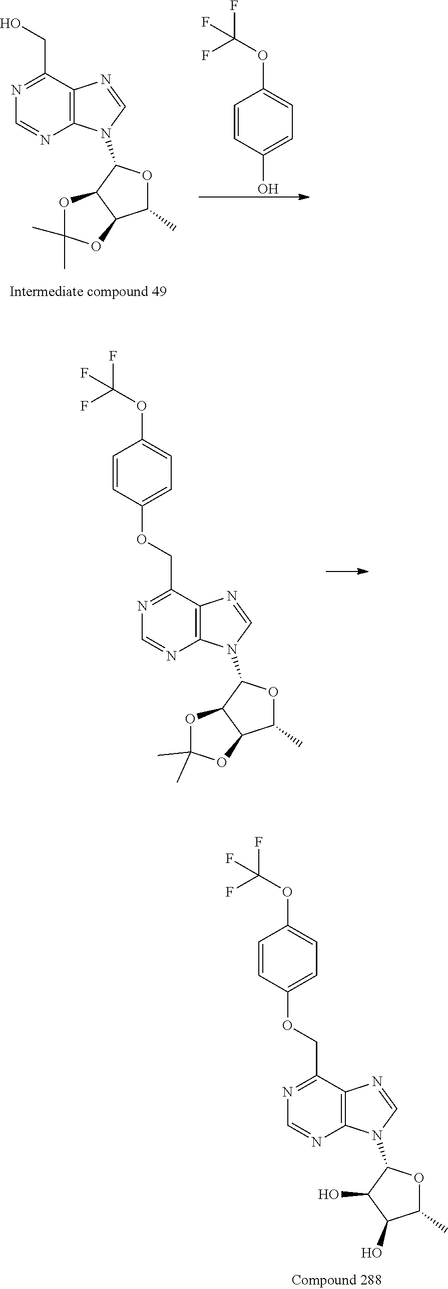 [SMILES: C[C@H]1O[C@@H](n2cnc3c(CO)ncnc32)[C@@H]2OC(C)(C)O[C@@H]21 ; Oc1ccc(OC(F)(F)F)cc1 ; C[C@H]1O[C@@H](n2cnc3c(COc4ccc(OC(F)(F)F)cc4)ncnc32)[C@@H]2OC(C)(C)O[C@@H]21 ; C[C@H]1O[C@@H](n2cnc3c(COc4ccc(OC(F)(F)F)cc4)ncnc32)[C@H](O)[C@@H]1O]

Compound 288 (8.24 mg, 30% yield) was synthesized from Intermediate Compound 49 and 4-(trifluoromethoxy)phenol following the protocols described for Compound 280.

LCMS: Rt=0.849 min; (ESI positive ion) m/z: 426.9 (M+H)+(calculated: 427.12).

1H NMR (400 MHz, DMSO-d6) δ=8.96 (s, 1H), 8.84 (s, 1H), 7.29 (d, J=9.0 Hz, 2H), 7.17-7.12 (m, 2H), 6.00 (d, J=5.0 Hz, 1H), 5.58 (s, 2H), 5.53 (d, J=5.4 Hz, 1H), 5.24 (br d, J=4.6 Hz, 1H), 4.79-4.72 (m, 1H), 4.08-3.98 (m, 2H), 1.33 (d, J=6.0 Hz, 3H).

### Compound 290:

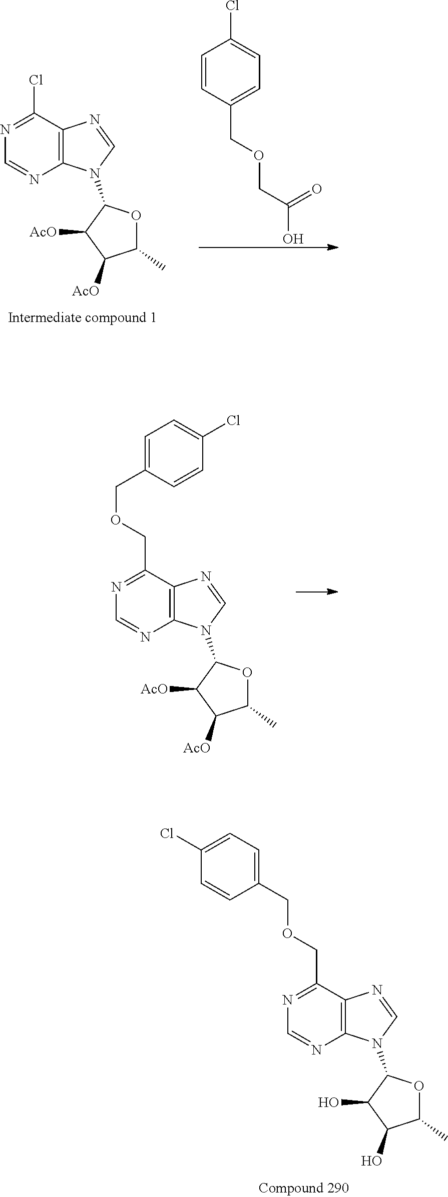 [SMILES: CC(=O)O[C@@H]1[C@H](OC(C)=O)[C@@H](C)O[C@H]1n1cnc2c(Cl)ncnc21 ; O=C(O)COCc1ccc(Cl)cc1 ; CC(=O)O[C@@H]1[C@H](OC(C)=O)[C@@H](C)O[C@H]1n1cnc2c(COCc3ccc(Cl)cc3)ncnc21 ; C[C@H]1O[C@@H](n2cnc3c(COCc4ccc(Cl)cc4)ncnc32)[C@H](O)[C@@H]1O]

Compound 290 (10.48 mg, 22% yield) was synthesized Intermediate Compound 1 and 2-((4-chlorobenzyl)oxy)acetic acid following the protocols described for Compound 285. LCMS: Rt=0.809 min; (ESI positive ion) m/z: 390.9 (M+H)+(calculated: 391.11).

1H NMR (400 MHz, DMSO-d6) δ=8.94 (s, 1H), 8.80 (s, 1H), 7.41 (s, 4H), 6.00 (d, J=5.0 Hz, 1H), 5.55 (d, J=5.6 Hz, 1H), 5.26 (br d, J=4.4 Hz, 1H), 4.97 (s, 2H), 4.74 (q, J=4.6 Hz, 1H), 4.67 (s, 2H), 4.05-3.99 (m, 2H), 1.33 (d, J=6.2 Hz, 3H).

### Compound 292:

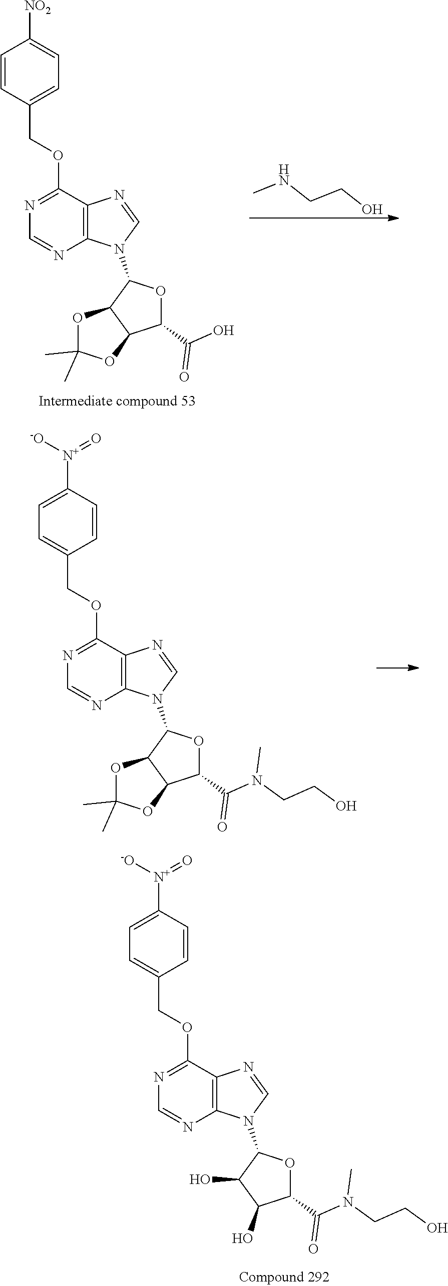

Compound 292 (26.26 mg, 57% yield) was synthesized from Intermediate Compound 53 and 2-(methylamino)ethan-1-ol following the protocols described for Compound 271.

LCMS: Rt=0.780 min; (ESI positive ion) m/z: 475.2 (M+H)+(calculated: 475.15).

1H NMR (400 MHz, DMSO-d6) δ=8.73 (s, 1H), 8.52 (s, 1H), 8.22 (d, J=8.8 Hz, 2H), 7.78 (d, J=8.4 Hz, 2H), 6.21 (d, J=5.5 Hz, 1H), 5.85 (s, 2H), 5.35-4.89 (m, 3H), 4.65 (t, J=5.1 Hz, 1H), 4.46-4.42 (m, 1H), 3.62-3.56 (m, 2H), 3.51-3.44 (m, 2H), 3.02 (br s, 3H).

### Compound 294:

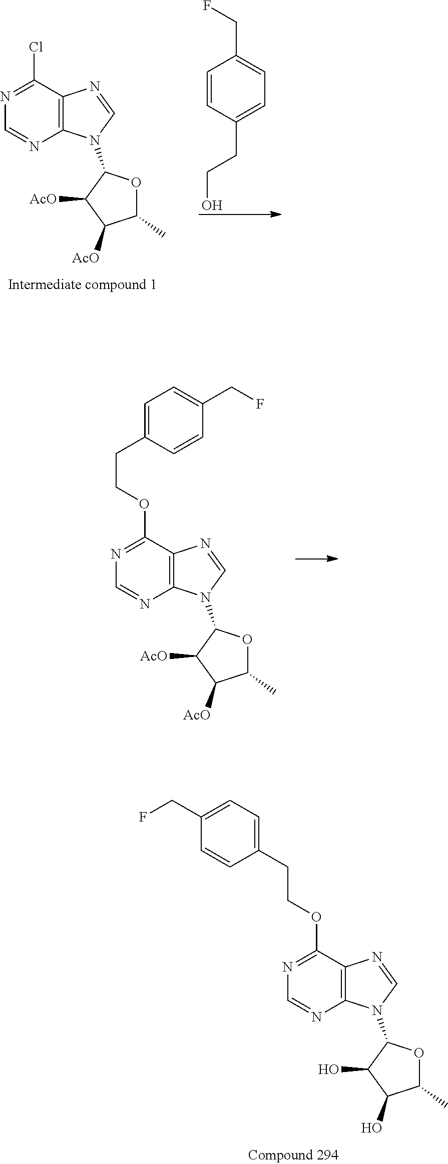 [SMILES: CC(=O)O[C@@H]1[C@H](OC(C)=O)[C@@H](C)O[C@H]1n1cnc2c(Cl)ncnc21 ; OCCc1ccc(CF)cc1 ; CC(=O)O[C@@H]1[C@H](OC(C)=O)[C@@H](C)O[C@H]1n1cnc2c(OCCc3ccc(CF)cc3)ncnc21 ; C[C@H]1O[C@@H](n2cnc3c(OCCc4ccc(CF)cc4)ncnc32)[C@H](O)[C@@H]1O]

Compound 294 (21.89 mg, 17% yield) was synthesized from Intermediate Compound 1 and 2-(4-(fluoromethyl)phenyl)ethan-1-ol following the protocols described for Compound 188.

LCMS: Rt=0.831 min; (ESI positive ion) m/z: 389.3 (M+H)+(calculated: 389.15).

1H NMR (400 MHz, DMSO-d6) δ=8.55 (d, J=8.4 Hz, 2H), 7.44-7.31 (m, 4H), 5.93 (d, J=4.9 Hz, 1H), 5.43 (s, 2H), 5.31 (s, 2H), 4.78 (t, J=6.7 Hz, 2H), 4.68 (t, J=4.3 Hz, 1H), 4.00 (br d, J=4.2 Hz, 2H), 3.17 (br t, J=6.7 Hz, 2H), 1.31 (br d, J=5.4 Hz, 3H).

### Compound 295:

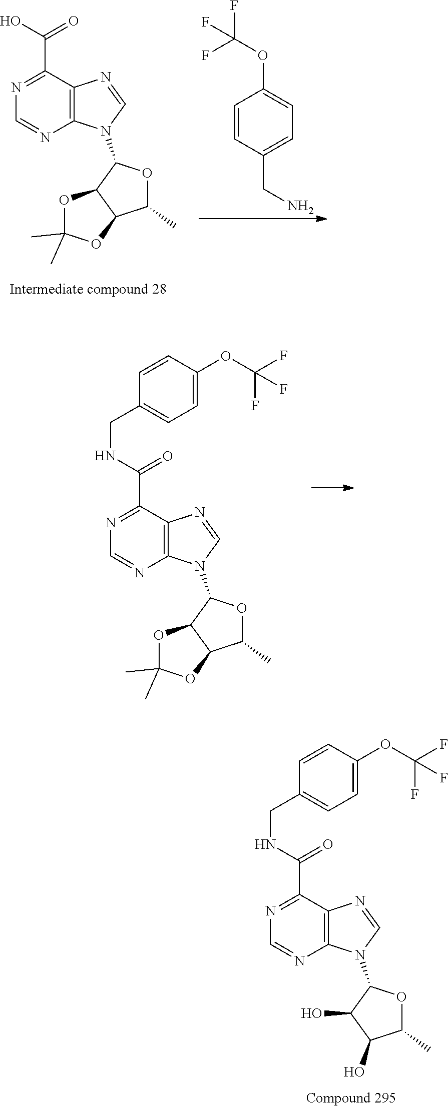 [SMILES: C[C@H]1O[C@@H](n2cnc3c(C(=O)O)ncnc32)[C@@H]2OC(C)(C)O[C@@H]21 ; NCc1ccc(OC(F)(F)F)cc1 ; C[C@H]1O[C@@H](n2cnc3c(C(=O)NCc4ccc(OC(F)(F)F)cc4)ncnc32)[C@@H]2OC(C)(C)O[C@@H]21 ; C[C@H]1O[C@@H](n2cnc3c(C(=O)NCc4ccc(OC(F)(F)F)cc4)ncnc32)[C@H](O)[C@@H]1O]

To a mixture of Intermediate Compound 28 (100 mg, 312.21 μmol) and (4-(trifluoromethoxy)phenyl)methanamine (71.61 mg, 374.65 μmol, 57.29 μL) in THE (10 mL) was added T3P (496.69 mg, 780.52 μmol, 464.20 μL, 50% purity) and DIEA (201.75 mg, 1.56 mmol, 271.90 μL) at 0° C. The mixture was stirred at 25° C. for 1 hr. The reaction mixture was then concentrated under vacuum and the resultant residue was purified by Prep-TLC (SiO2, Petroleum ether:Ethyl acetate=1:1) to afford the corresponding amide compound (115 mg, 68% yield) as a colorless oil.

LCMS: Rt=0.465 min; (ESI positive ion) m/z: 494.1 (M+H)+(calculated: 494.16).

A solution of the above amide compound (115 mg, 233.06 μmol) in formic acid (4 mL) and water (1 mL) was stirred at 0° C. for 6 hr. NH3·H2O was added to the reaction mixture at 0° C. until the pH reach 7. The mixture was then filtered and concentrated under reduced pressure to give a residue, which was dissolved in MeOH and purified by Prep-HPLC (column: Phenomenex C18 150×25 mm×10 μm; mobile phase: [water (ammonia hydroxide v/v)-ACN]; B %: 22%-52%, 8 min) to afford Compound 295 as a colorless gum.

LCMS: Rt=0.373 min; (ESI positive ion) m/z: 454.1 (M+H)+(calculated: 454.13).

1H NMR (400 MHz, DMSO-d6) δ=9.53 (t, J=6.2 Hz, 1H), 9.06 (s, 1H), 8.94 (s, 1H), 7.52 (d, J=8.8 Hz, 2H), 7.34 (d, J=8.0 Hz, 2H), 6.04 (d, J=4.8 Hz, 1H), 5.53 (d, J=5.6 Hz, 1H), 5.24 (d, J=5.2 Hz, 1H), 4.72 (q, J=5.0 Hz, 1H), 4.60 (d, J=6.4 Hz, 2H), 4.06-4.00 (m, 2H), 1.34 (d, J=6.2 Hz, 3H).

### Compound 296:

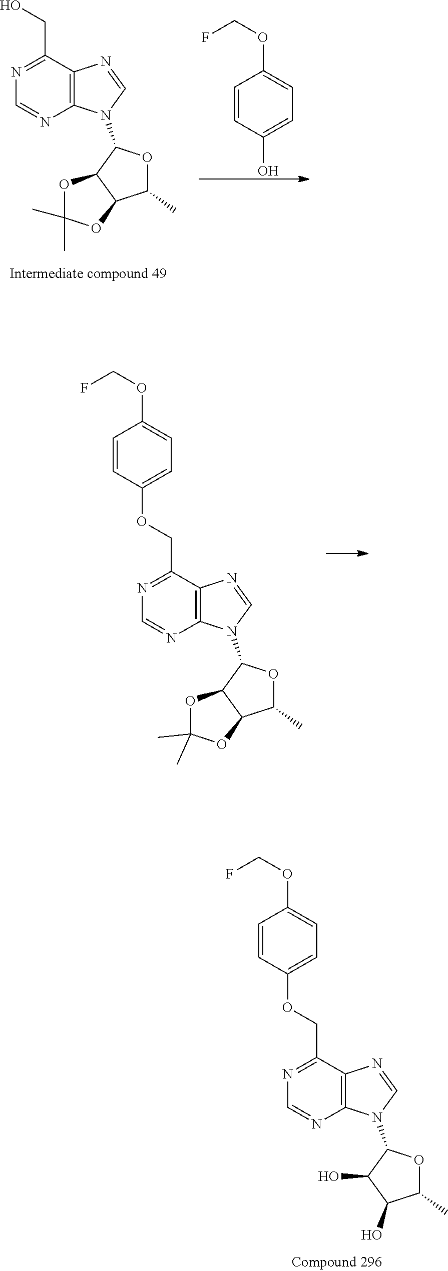 [SMILES: C[C@H]1O[C@@H](n2cnc3c(CO)ncnc32)[C@@H]2OC(C)(C)O[C@@H]21 ; Oc1ccc(OCF)cc1 ; C[C@H]1O[C@@H](n2cnc3c(COc4ccc(OCF)cc4)ncnc32)[C@@H]2OC(C)(C)O[C@@H]21 ; C[C@H]1O[C@@H](n2cnc3c(COc4ccc(OCF)cc4)ncnc32)[C@H](O)[C@@H]1O]

Compound 296 (3.1 mg, 7% yield) was synthesized from Intermediate Compound 49 and 4-(fluoromethoxy)phenol following the protocols described for Compound 233.

LCMS: Rt=0.775 min; (ESI positive ion) m/z: 390.9 (M+H)+(calculated: 391.13).

1H NMR (400 MHz, DMSO-d6) δ=8.95 (s, 1H), 8.83 (s, 1H), 7.03 (s, 4H), 6.00 (br d, J=4.8 Hz, 1H), 5.82 (s, 1H), 5.69 (s, 1H), 5.61-5.54 (m, 1H), 5.51 (s, 2H), 5.28 (br s, 1H), 4.75 (br s, 1H), 4.02 (br d, J=3.8 Hz, 2H), 1.33 (br d, J=5.6 Hz, 3H).

### Compound 298:

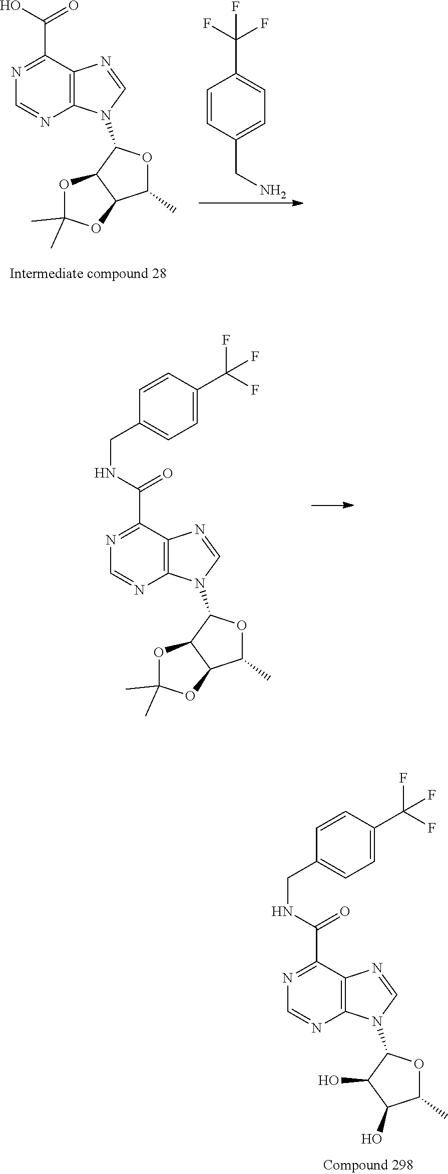 [SMILES: C[C@H]1O[C@@H](n2cnc3c(C(=O)O)ncnc32)[C@@H]2OC(C)(C)O[C@@H]21 ; NCc1ccc(C(F)(F)F)cc1 ; C[C@H]1O[C@@H](n2cnc3c(C(=O)NCc4ccc(C(F)(F)F)cc4)ncnc32)[C@@H]2OC(C)(C)O[C@@H]21 ; C[C@H]1O[C@@H](n2cnc3c(C(=O)NCc4ccc(C(F)(F)F)cc4)ncnc32)[C@H](O)[C@@H]1O]

To a solution of Intermediate Compound 28 (100 mg, 312.21 μmol) in THF (10 mL) was added (4-(trifluoromethyl)phenyl)methanamine (65.62 mg, 374.65 μmol, 53.35 μL), T3P (496.69 mg, 780.52 μmol, 464.20 μL, 50% purity), and DIEA (201.75 mg, 1.56 mmol, 271.90 μL) at 0° C. The mixture was stirred at 25° C. for 1 hr. The reaction mixture was then purified by Prep-TLC (SiO2, Ethyl acetate:Petroleum ether=1:1) to afford the corresponding amide compound (95 mg, 63% yield) as a red gum.

LCMS: Rt=0.569 min; (ESI positive ion) m/z: 478.2 (M+H)+(calculated: 478.16).

To a mixture of the above amide compound (95 mg, 198.98 μmol) in formic acid (4 mL) and water (1 mL) was stirred at 0° C. for 6 hr. NH3·H2O was added to the reaction mixture at 0° C. until the pH reached 7. The reaction was then filtered and concentrated under reduced pressure to give a residue, which was dissolved in MeOH and purified by Prep-HPLC (Phenomenex C18 150×25 mm×10 μm; mobile phase: [water (NH4HCO3)-ACN]; B %: 20%-50%, 8 min) to afford Compound 298 (35.84 mg, 41% yield) as a white solid.

LCMS: Rt=0.490 min; (ESI positive ion) m/z: 438.1 (M+H)+(calculated: 438.13).

1H NMR (400 MHz, DMSO-d6) δ=9.60 (t, J=6.4 Hz, 1H), 9.08 (s, 1H), 8.96 (s, 1H), 7.73 (d, J=8.2 Hz, 2H), 7.62 (d, J=8.0 Hz, 2H), 6.05 (d, J=4.8 Hz, 1H), 5.54 (d, J=5.6 Hz, 1H), 5.25 (d, J=5.2 Hz, 1H), 4.73 (q, J=5.0 Hz, 1H), 4.68 (br d, J=6.0 Hz, 2H), 4.04 (t, J=5.6 Hz, 2H), 1.35 (d, J=6.2 Hz, 3H)).

### Compound 301:

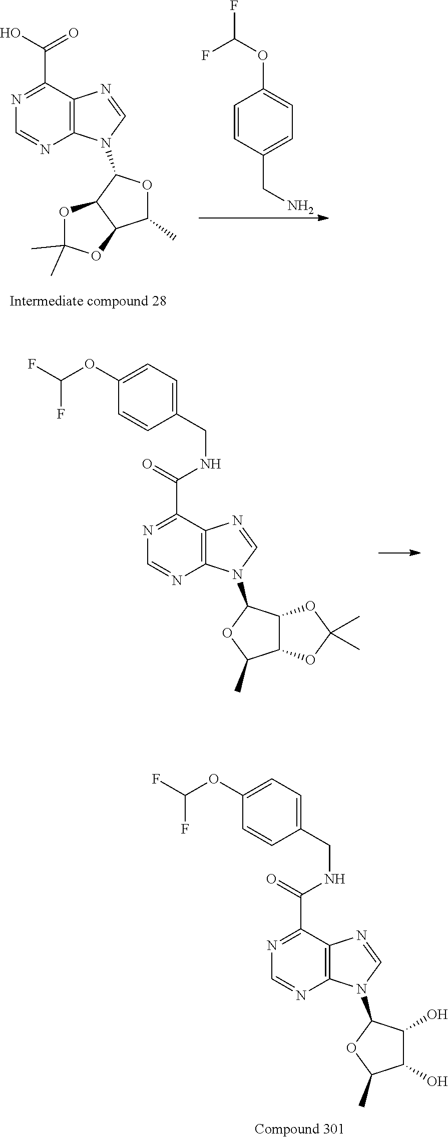 [SMILES: C[C@H]1O[C@@H](n2cnc3c(C(=O)O)ncnc32)[C@@H]2OC(C)(C)O[C@@H]21 ; NCc1ccc(OC(F)F)cc1 ; C[C@H]1O[C@@H](n2cnc3c(C(=O)NCc4ccc(OC(F)F)cc4)ncnc32)[C@@H]2OC(C)(C)O[C@@H]21 ; C[C@H]1O[C@@H](n2cnc3c(C(=O)NCc4ccc(OC(F)F)cc4)ncnc32)[C@H](O)[C@@H]1O]

Compound 301 (8.0 mg, 8% yield) was synthesized from Intermediate Compound 28 and (4-(difluoromethoxy)phenyl)methanamine following the protocols described for Compound 295.

LCMS: Rt=0.812 min; (ESI positive ion) m/z: 436.0 (M+H)+(calculated: 436.14).

1H NMR (400 MHz, MeOD-d4) δ=9.08 (s, 1H), 8.76 (s, 1H), 7.49 (br d, J=8.4 Hz, 2H), 7.14 (br d, J=8.8 Hz, 2H), 6.14 (br d, J=4.2 Hz, 1H), 4.73 (br s, 3H), 4.31-4.09 (m, 2H), 1.55-1.41 (m, 3H).

### Compound 302:

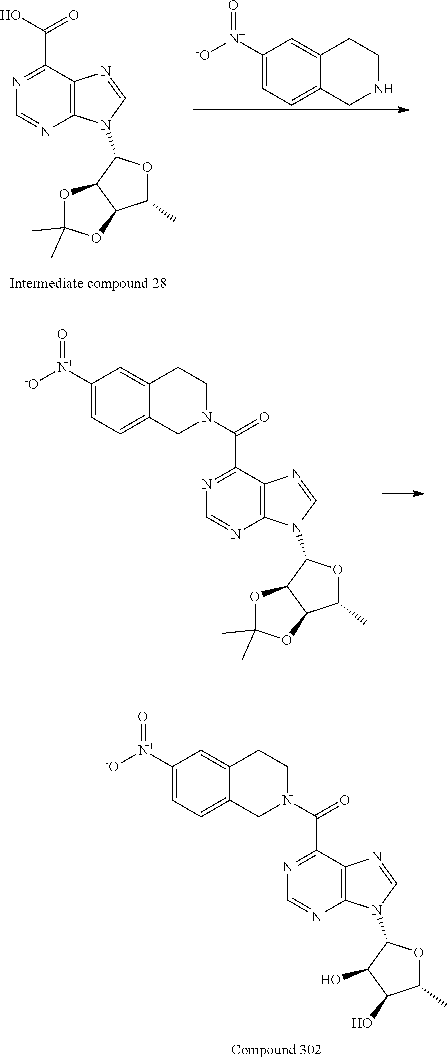

Compound 302 (25.6 mg, 20% yield) was synthesized from Intermediate Compound 28 and 6-nitro-1,2,3,4-tetrahydroisoquinoline following the protocols described for Compound 360.

LCMS: Rt=0.680 min; (ESI positive ion) m/z: 441.2 (M+H)+(calculated: 441.14).

1H NMR (400 MHz, DMSO-d6) δ=9.04-9.00 (m, 1H), 8.88-8.84 (m, 1H), 8.14-8.11 (m, 1H), 8.10-7.95 (m, 1H), 7.65-7.25 (m, 1H), 6.05-6.00 (m, 1H), 5.58 (br s, 1H), 5.30 (br s, 1H), 5.05 (s, 1H), 4.74 (br s, 1H), 4.60 (s, 1H), 4.08-4.00 (m, 3H), 3.52 (br t, J=5.8 Hz, 1H), 3.10 (br t, J=5.9 Hz, 1H), 2.95 (br t, J=5.7 Hz, 1H), 1.35 (dd, J=2.3, 6.1 Hz, 3H).

### Compound 303:

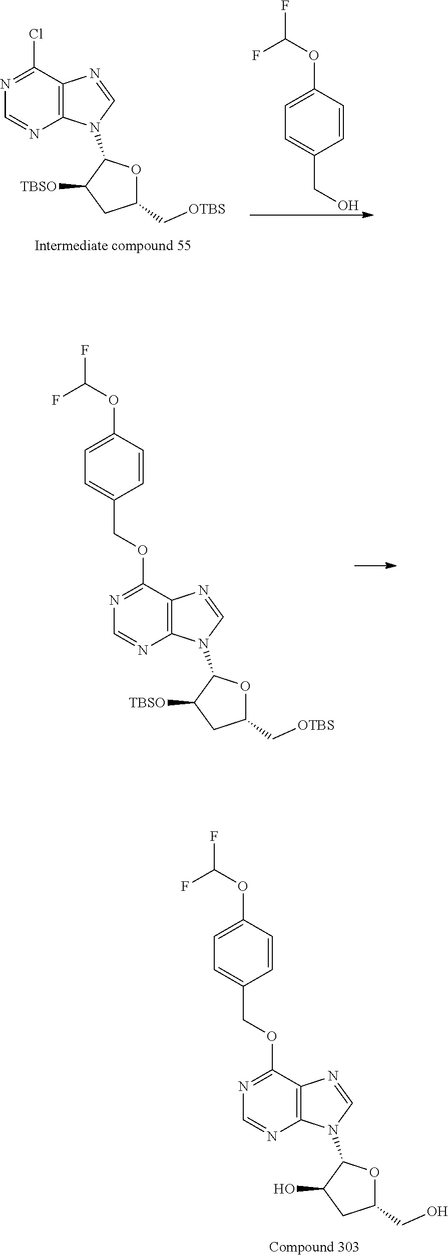 [SMILES: CC(C)(C)[Si](C)(C)OC[C@@H]1C[C@@H](O[Si](C)(C)C(C)(C)C)[C@H](n2cnc3c(Cl)ncnc32)O1 ; OCc1ccc(OC(F)F)cc1 ; CC(C)(C)[Si](C)(C)OC[C@@H]1C[C@@H](O[Si](C)(C)C(C)(C)C)[C@H](n2cnc3c(OCc4ccc(OC(F)F)cc4)ncnc32)O1 ; OC[C@@H]1C[C@@H](O)[C@H](n2cnc3c(OCc4ccc(OC(F)F)cc4)ncnc32)O1]

To a solution of Intermediate Compound 55 (150 mg, 300.49 μmol, 1 eq) in toluene (3 mL) was added Cs2CO3 (244.76 mg, 751.21 μmol, 2.5 eq), (4-(difluoromethoxy)phenyl)methanol (78.49 mg, 450.73 μmol, 1.5 eq), Xantphos (34.77 mg, 60.10 mol, 0.2 eq), and Pd2(dba)3 (27.52 mg, 30.05 μmol, 0.1 eq). The mixture was stirred at 80° C. for 0.5 hr under N2 (balloon). The solvent was removed under reduced pressure to give a residue, which was purified by column chromatography (SiO2, Petroleum ether/Ethyl acetate=10/1 to 7/1) to afford the corresponding ether compound (190 mg, 298.33 μmol, 99.28% yield) as a yellow oil.

LCMS: Rt=0.981 min; (ESI positive ion) m/z: 637.3 (M+H)+(calculated: 637.30).

To a solution of the above ether compound (185 mg, 290.48 μmol, 1 eq) in MeOH (3 mL) was added ammonium fluoride (340 mg, 9.18 mmol, 31.60 eq). The mixture was stirred at 65° C. for 3 hr. The solvent was removed under reduced pressure to give the residue, which was purified by Prep-HPLC (column: Phenomenex C18 75×30 mm×3 μm; mobile phase: [water(FA)-ACN]; B %: 25%-55%, 7 min). The purified solution was lyophilized to afford Compound 303 (44.76 mg, 38% yield) as a white solid.

LCMS: Rt=0.856 min; (ESI positive ion) m/z: 409.1 (M+H)+(calculated: 409.12).

1H NMR (400 MHz, DMSO-d6) δ=8.67-8.53 (m, 2H), 7.58 (d, J=8.6 Hz, 2H), 7.24 (t, J=74 Hz, 1H), 7.21 (d, J=8.6 Hz, 2H), 5.99 (d, J=1.7 Hz, 1H), 5.72 (d, J=4.2 Hz, 1H), 5.67-5.58 (m, 2H), 5.06 (t, J=5.4 Hz, 1H), 4.58 (br s, 1H), 4.43-4.35 (m, 1H), 3.71 (ddd, J=3.4, 5.3, 11.9 Hz, 1H), 3.53 (td, J=4.7, 12.0 Hz, 1H), 2.31-2.20 (m, 1H), 1.91 (ddd, J=2.5, 6.1, 13.1 Hz, 1H).

### Compound 304:

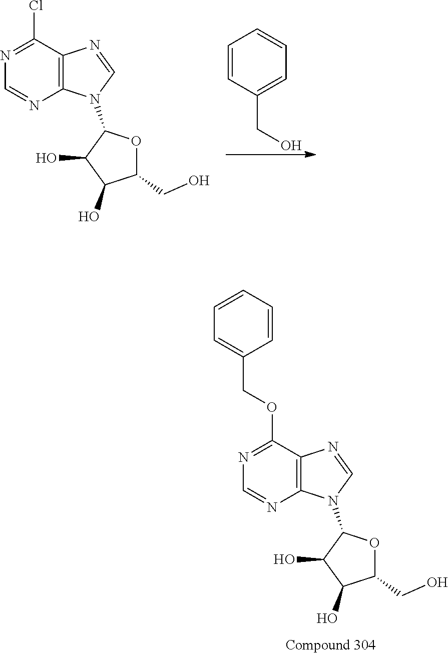 [SMILES: OC[C@H]1O[C@@H](n2cnc3c(Cl)ncnc32)[C@H](O)[C@@H]1O ; OCc1ccccc1 ; OC[C@H]1O[C@@H](n2cnc3c(OCc4ccccc4)ncnc32)[C@H](O)[C@@H]1O]

To a solution of (2R,3R,4S,5R)-2-(6-chloro-9H-purin-9-yl)-5-(hydroxymethyl)tetrahydrofuran-3,4-diol (150 mg, 523.25 μmol, 1 eq) in EtOH (3 mL) was added TEA (423.58 mg, 4.19 mmol, 582.64 μL, 8 eq) and phenylmethanamine (112.13 mg, 1.05 mmol, 114.07 μL, 2 eq). The mixture was stirred at 80° C. for 2 hr. The mixture was then filtered and concentrated under vacuum. The resultant residue was purified by Prep-HPLC (column: Unisil 3-100 C18 Ultra 150×50 mm×3 μm; mobile phase: [water(FA)-ACN]; B %: 10%-40%, 10 min) to afford Compound 304 (110.44 mg, 59% yield) as a white solid.

LCMS: Rt=0.680 min; (ESI positive ion) m/z: 357.8 (M+H)+(calculated: 357.14).

1H NMR (400 MHz, DMSO-d6) δ=8.46 (br d, J=0.8 Hz, 1H), 8.38 (s, 1H), 8.20 (s, 1H), 7.34-7.27 (m, 4H), 7.23-7.17 (m, 1H), 5.89 (d, J=6.2 Hz, 1H), 5.44 (d, J=6.2 Hz, 1H), 5.37 (dd, J=4.6, 7.0 Hz, 1H), 5.19 (d, J=4.6 Hz, 1H), 4.71 (br s, 2H), 4.61 (q, J=5.8 Hz, 1H), 4.17-4.12 (m, 1H), 3.96 (q, J=3.4 Hz, 1H), 3.67 (td, J=4.0, 12.1 Hz, 1H), 3.58-3.51 (m, 1H).

### Compound 305:

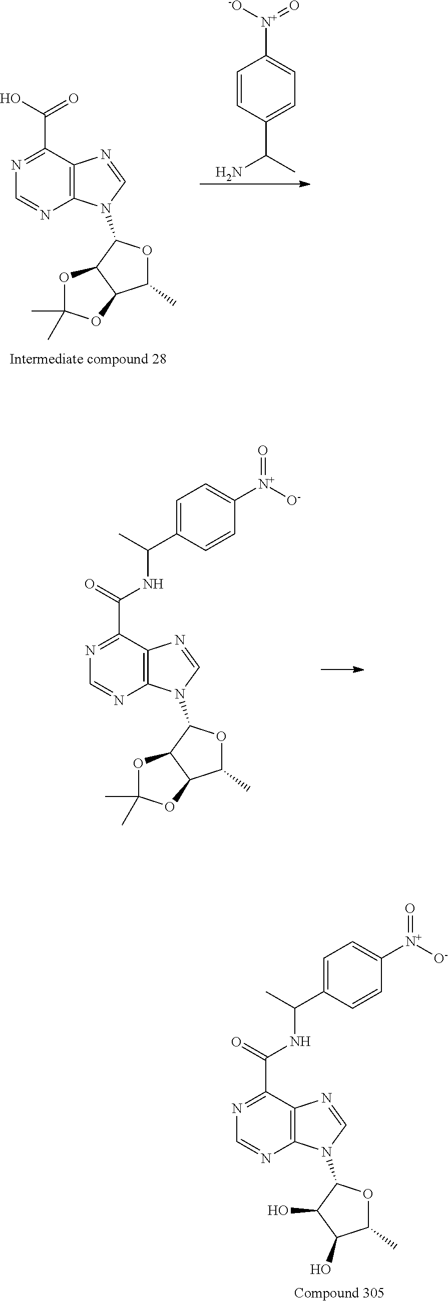

To a solution of Intermediate Compound 28 (100 mg, 312.21 μmol, 1 eq) and 1-(4-nitrophenyl)ethan-1-amine (77.82 mg, 468.31 μmol, 1.5 eq) in THF (5 mL) was added T3P (496.69 mg, 780.52 μmol, 464.20 μL, 50% purity, 2.5 eq) and DIEA (201.75 mg, 1.56 mmol, 271.90 μL, 5 eq) at 0° C. The mixture was stirred at 20° C. for 2 hr. The solvent was removed under reduced pressure to give a residue, which was purified by Prep-TLC (SiO2, Petroleum ether:ethyl acetate=0:1) to afford the corresponding amide compound (121 mg, 83% yield) as a white solid. LCMS: Rt=0.423 min; (ESI positive ion) m/z: 469.2 (M+H)+(calculated: 469.18).

A solution of the above amide compound (110 mg, 234.81 μmol, 1 eq) in formic acid/water (4/1 V/V) (3 mL) was stirred at 0° C. for 9 hr. NH3·H2O was added to the reaction mixture at 0° C. until a pH of 7 was achieved. The mixture was then diluted with H2O (20 ml×3) and extracted with ethyl acetate (20 mL×3). The combined organic layers were concentrated under reduced pressure to give a residue, which was purified by Prep-HPLC (column: Waters xbridge 150×25 mm 10 μm; mobile phase: [water (NH4HCO3)-ACN]; B %: 14%-44%, 8 min) to afford Compound 305 (35.72 mg, 35% yield) as a white solid.

LCMS: Rt=0.410 min; (ESI positive ion) m/z: 429.1 (M+H)+(calculated: 429.14).

1H NMR (400 MHz, DMSO-d6) δ=9.50 (d, J=7.8 Hz, 1H), 9.07 (s, 1H), 8.95 (s, 1H), 8.22 (d, J=8.8 Hz, 2H), 7.75 (d, J=8.8 Hz, 2H), 6.04 (d, J=4.8 Hz, 1H), 5.53 (d, J=5.6 Hz, 1H), 5.34 (quin, J=7.4 Hz, 1H), 5.24 (d, J=5.2 Hz, 1H), 4.72 (q, J=5.0 Hz, 1H), 4.09-3.99 (m, 2H), 1.56 (d, J=7.0 Hz, 3H), 1.34 (d, J=6.0 Hz, 3H).

### Compound 309:

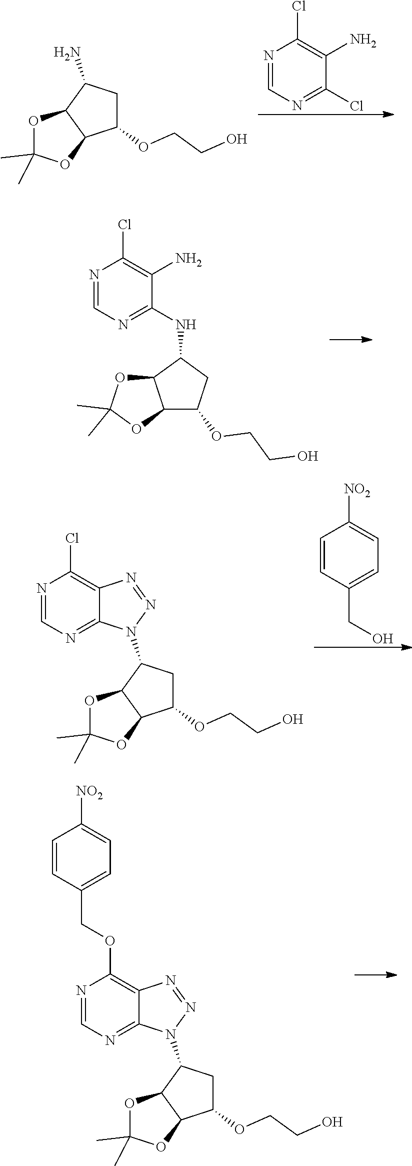

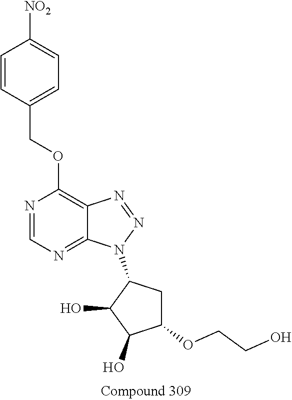 [SMILES: O=[N+]([O-])c1ccc(COc2ncnc3c2nnn3[C@@H]2C[C@H](OCCO)[C@@H](O)[C@H]2O)cc1]

A mixture of 2-(((3aR,4S,6R,6aS)-6-amino-2,2-dimethyltetrahydro-4H-cyclopenta[d][1,3]dioxol-4-yl)oxy)ethan-1-ol (10 g, 27.22 mmol, 1 eq) and 4,6-dichloropyrimidin-5-amine (5.89 g, 35.93 mmol, 1.32 eq) in DIPEA (30 mL) was degassed and purged with N2 3 times. The mixture was stirred at 120° C. for 12 hr under N2 atmosphere. The reaction mixture was then concentrated under reduced pressure to give a residue, which was purified by flash silica gel chromatography (ISCO®; 40 g SepaFlash® Silica Flash Column, Eluent of 0-70% Ethyl acetate/Petroleum ether gradient @100 mL/min) to afford the conjugated intermediate compound (5.2 g, 53% yield) as a yellow oil.

LCMS: Rt=0.283 min; (ESI positive ion) m/z: 344.8 (M+H)+(calculated: 345.13).

1H NMR (400 MHz, DMSO-d6) δ=7.79 (s, 1H), 6.43 (br d, J=7.4 Hz, 1H), 4.99 (s, 2H), 4.55-4.50 (m, 1H), 4.50-4.45 (m, 1H), 4.36 (br s, 1H), 3.88 (br s, 1H), 3.59-3.43 (m, 5H), 2.26-2.16 (m, 1H), 1.87 (br d, J=14.0 Hz, 1H), 1.37 (s, 3H), 1.21 (s, 3H).

To a solution of the above conjugated intermediate compound (1 g, 2.90 mmol, 1 eq) in toluene (10 mL) was added AcOH (844.72 mg, 14.07 mmol, 804.49 μL, 4.85 eq) at 30° C., and the mixture was then cooled to a temperature of 0° C. Aqueous sodium nitrite (600.32 mg, 8.70 mmol, 3 eq) solution was slowly added to the mixture and stirred at 0° C. for 1 hr. K2CO3 was added to the reaction mixture at 0° C. until the pH reached approximately 7. The mixture was then extracted with ethyl acetate (20 mL×2). The combined organic layers were dried over Na2SO4, filtered and concentrated under reduced pressure to give a residue, which was purified by flash silica gel chromatography (ISCO®; 4 g SepaFlash® Silica Flash Column, Eluent of 0-65% Ethyl acetate/Petroleum ether gradient @20 mL/min) to afford the corresponding triazole compound (600 mg, 50% yield) as a yellow oil.

LCMS: Rt=0.427 min; (ESI positive ion) m/z: 356.0 (M+H)+(calculated: 356.10).

1H NMR (400 MHz, CDCl₃) δ=9.06-8.85 (m, 1H), 5.65-5.59 (m, 1H), 5.32-5.26 (m, 1H), 4.82 (br d, J=6.4 Hz, 1H), 4.68 (br s, 2H), 4.07-4.00 (m, 1H), 3.81-3.73 (m, 1H), 3.70-3.47 (m, 2H), 2.79-2.40 (m, 3H), 1.56 (s, 3H), 1.37 (s, 3H).

To a solution of the above triazole compound (200 mg, 562.15 μmol, 1 eq), (4-nitrophenyl)methanol (103.30 mg, 674.58 μmol, 1.2 eq), Cs2CO3 (457.90 mg, 1.41 mmol, 2.5 eq) and Xantphos (65.05 mg, 112.43 μmol, 0.2 eq) in toluene (4 mL) was added Pd2(dba)3 (51.48 mg, 56.22 μmol, 0.1 eq). The mixture was stirred at 80° C. for 0.5 hr under N2 atmosphere. The reaction mixture was concentrated under reduced pressure to give a residue, which was purified by Prep-TLC (SiO2, Ethyl acetate:Petroleum ether=2:1) to afford the corresponding ether compound (125 mg, 34% yield) as a yellow oil.

LCMS: Rt=0.526 min; (ESI positive ion) m/z: 473.1 (M+H)+(calculated: 473.17).

A mixture of the above ether compound (125 mg, 190.50 μmol, 72% purity, 1 eq) in formic acid (0.8 mL) and water (0.2 mL) was degassed and purged with N2 3 times. The mixture was stirred at 0° C. for 4 hr under N2 atmosphere. The mixture was concentrated under vacuum and the resultant residue was treated with K2CO3 (30 mg)/MeOH (1 mL). The mixture was stirred at 20° C. for 10 min and then concentrated under reduced pressure to give a residue. The residue was purified by Prep-TLC (SiO2, Ethyl acetate:Ethanol=20:1) to afford Compound 309 (12.93 mg, 15% yield) as a white solid.

LCMS: Rt=0.411 min; (ESI positive ion) m/z: 433.0 (M+H)+(calculated: 433.14).

1H NMR (400 MHz, DMSO-d6) δ=8.78 (s, 1H), 8.29 (d, J=8.8 Hz, 2H), 7.82 (d, J=8.8 Hz, 2H), 5.90 (s, 2H), 5.21-5.15 (m, 2H), 5.12 (d, J=4.0 Hz, 1H), 4.63-4.57 (m, 2H), 3.97 (br s, 1H), 3.83-3.74 (m, 1H), 3.53-3.50 (m, 3H), 3.17 (d, J=5.4 Hz, 1H), 2.80-2.69 (m, 1H), 2.16-2.08 (m, 1H).

### Compound 312:

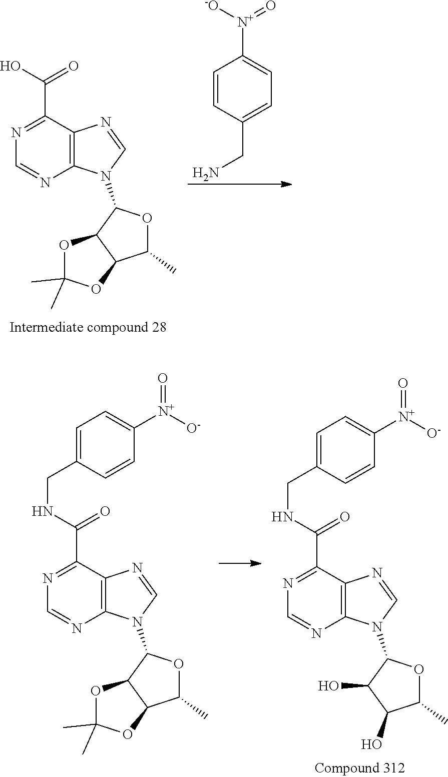

Compound 312 (4.6 mg, 11% yield) has been synthesized from Intermediate Compound 28 and (4-nitrophenyl)methanamine following the protocols described for Compound 295.

LCMS: Rt=0.771 min; (ESI positive ion) m/z: 415.0 (M+H)+(calculated: 415.13).

1H NMR (400 MHz, MeOD-d4) δ=9.08 (s, 1H), 8.76 (s, 1H), 7.49 (br d, J=8.4 Hz, 2H), 7.14 (br d, J=8.8 Hz, 2H), 6.14 (br d, J=4.2 Hz, 1H), 4.73 (br s, 3H), 4.31-4.09 (m, 2H), 1.55-1.41 (m, 3H).

### Compound 317:

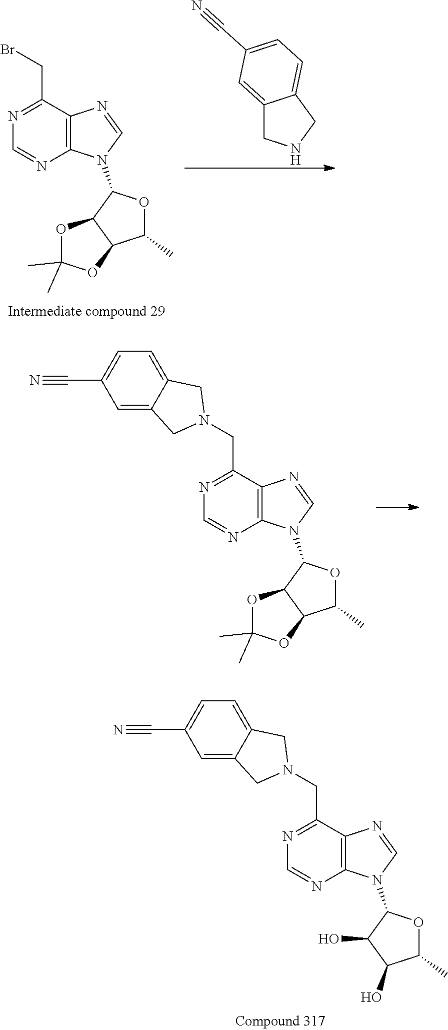

To a solution of Intermediate Compound 29 (50 mg, 135.42 μmol, 1 eq) in DMF (1 mL) was added isoindoline-5-carbonitrile hydrochloride (26.91 mg, 148.96 μmol, 1.1 eq, HCl) and TEA (34.26 mg, 338.55 μmol, 47.12 μL, 2.5 eq). The mixture was stirred at 20° C. for 1 hr. The reaction mixture was diluted with H2O (10 mL) and extracted with ethyl acetate (20 mL×2). The combined organic layers were washed with brine (20 mL), dried over Na2SO4 filtered and concentrated under reduced pressure to give a residue. The residue was purified by Prep-TLC (SiO2, Petroleum ether:ethyl acetate=0:1) to afford the corresponding amine compound (52 mg, 89% yield) as a gray solid.

LCMS: Rt=0.327 min; (ESI positive ion) m/z: 433.1 (M+H)+(calculated: 433.19).

A solution of the above amine compound (50 mg, 115.61 μmol, 1 eq) in formic acid/water (4/1 V/V) (2 mL) was stirred at 20° C. for 1 hr. The reaction mixture was concentrated under reduced pressure to give a residue, which was purified by Prep-HPLC (column: Phenomenex luna C18 150×25 mm×10 μm; mobile phase: [water(FA)-ACN]; B %: 1%-20%, 10 min) to afford Compound 317 (5.37 mg, 12% yield) as a white solid.

LCMS: Rt=0.210 min; (ESI positive ion) m/z: 393.1 (M+H)+(calculated: 393.16).

1H NMR (400 MHz, DMSO-d6) δ=8.92 (s, 1H), 8.77 (s, 1H), 8.31 (s, 1H), 7.70 (s, 1H), 7.65 (d, J=7.8 Hz, 1H), 7.44 (d, J=7.8 Hz, 1H), 5.99 (d, J=5.0 Hz, 1H), 5.68-5.09 (m, 2H), 4.74 (t, J=4.6 Hz, 1H), 4.38 (s, 2H), 4.13 (s, 2H), 4.09 (s, 2H), 4.02 (br d, J=4.8 Hz, 2H), 1.33 (d, J=6.2 Hz, 3H).

### Compound 318:

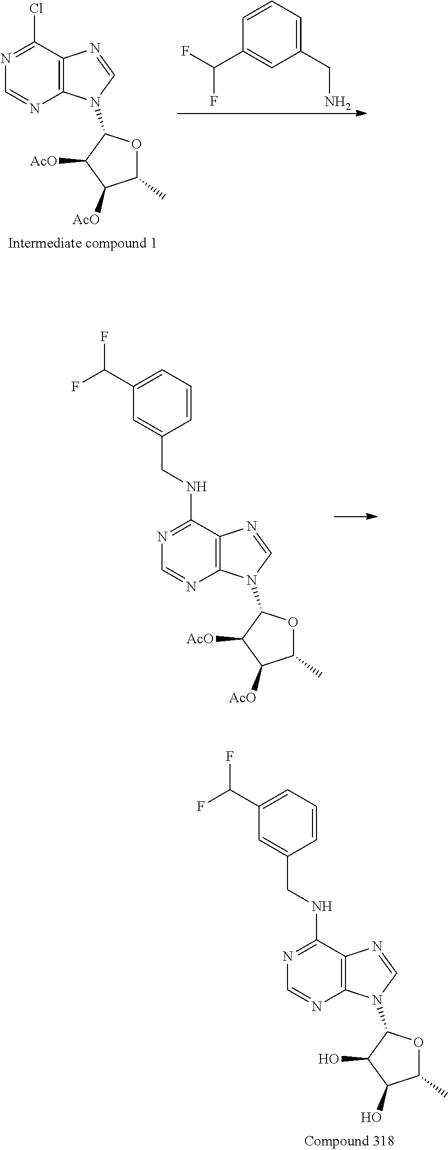 [SMILES: CC(=O)O[C@@H]1[C@H](OC(C)=O)[C@@H](C)O[C@H]1n1cnc2c(Cl)ncnc21 ; NCc1cccc(C(F)F)c1 ; CC(=O)O[C@@H]1[C@H](OC(C)=O)[C@@H](C)O[C@H]1n1cnc2c(NCc3cccc(C(F)F)c3)ncnc21 ; C[C@H]1O[C@@H](n2cnc3c(NCc4cccc(C(F)F)c4)ncnc32)[C@H](O)[C@@H]1O]

Compound 318 (34.21 mg, 62% yield) was synthesized from Intermediate Compound 1 and (3-(difluoromethyl)phenyl)methanamine following the protocols described for Compound 254.

LCMS: Rt=0.776 min; (ESI positive ion) m/z: 392.2 (M+H)+(calculated: 392.15).

1H NMR (400 MHz, DMSO-d6) δ=8.56-8.43 (m, 1H), 8.36 (s, 1H), 8.22 (s, 1H), 7.58-7.39 (m, 5H), 7.18-6.83 (m, 1H), 5.85 (d, J=4.8 Hz, 1H), 5.43 (br d, J=5.0 Hz, 1H), 5.16 (br d, J=3.2 Hz, 1H), 4.75 (br d, J=2.0 Hz, 2H), 4.67 (br d, J=4.2 Hz, 1H), 4.02-3.86 (m, 2H), 1.30 (br d, J=6.0 Hz, 3H).

### Compound 319:

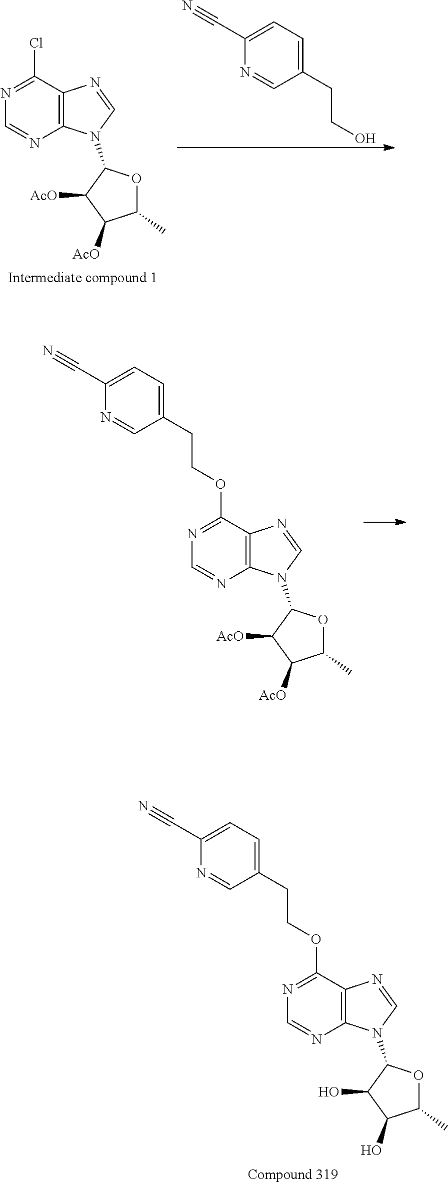 [SMILES: CC(=O)O[C@@H]1[C@H](OC(C)=O)[C@@H](C)O[C@H]1n1cnc2c(Cl)ncnc21 ; N#Cc1ccc(CCO)cn1 ; CC(=O)O[C@@H]1[C@H](OC(C)=O)[C@@H](C)O[C@H]1n1cnc2c(OCCc3ccc(C#N)nc3)ncnc21 ; C[C@H]1O[C@@H](n2cnc3c(OCCc4ccc(C#N)nc4)ncnc32)[C@H](O)[C@@H]1O]

Compound 319 (16.2 mg, 28% yield) was synthesized from Intermediate Compound 1 and 5-(2-hydroxyethyl)picolinonitrile following the protocols described for Compound 188.

LCMS: Rt=0.735 min; (ESI positive ion) m/z: 383.2 (M+H)+(calculated: 383.14).

1H NMR (400 MHz, DMSO-d6) δ=8.66 (d, J=1.8 Hz, 1H), 8.57 (s, 1H), 8.54 (s, 1H), 8.30-8.26 (m, 1H), 8.25-8.19 (m, 1H), 5.93 (d, J=5.0 Hz, 1H), 5.48 (br d, J=4.8 Hz, 1H), 5.21 (br s, 1H), 4.87 (t, J=6.4 Hz, 2H), 4.67 (br d, J=4.0 Hz, 1H), 4.02-3.96 (m, 2H), 3.38-3.35 (m, 2H), 1.31 (d, J=6.0 Hz, 3H).

### Compound 326:

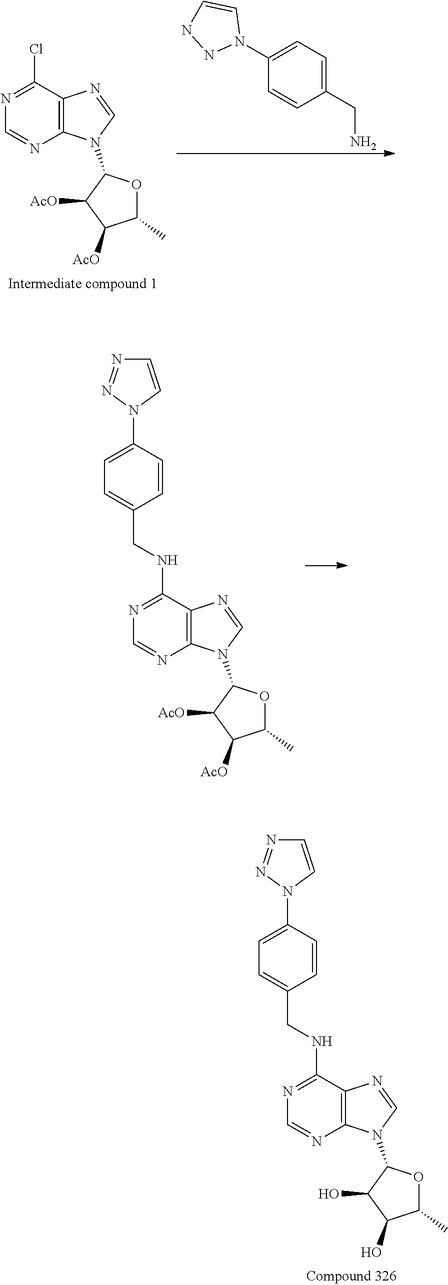 [SMILES: CC(=O)O[C@@H]1[C@H](OC(C)=O)[C@@H](C)O[C@H]1n1cnc2c(Cl)ncnc21 ; NCc1ccc(N2C=CNN2)cc1 ; CC(=O)O[C@@H]1[C@H](OC(C)=O)[C@@H](C)O[C@H]1n1cnc2c(NCc3ccc(-n4ccnn4)cc3)ncnc21 ; C[C@H]1O[C@@H](n2cnc3c(NCc4ccc(-n5ccnn5)cc4)ncnc32)[C@H](O)[C@@H]1O]

Compound 326 (35.33 mg, 61% yield) was synthesized from Intermediate Compound 1 and (4-(1H-1,2,3-triazol-1-yl)phenyl)methanamine following the protocols described for Compound 254.

LCMS: Rt=0.715 min; (ESI positive ion) m/z: 409.3 (M+H)+(calculated: 409.17).

1H NMR (400 MHz, DMSO-d6) δ=8.76 (d, J=1.0 Hz, 1H), 8.57-8.45 (m, 1H), 8.37 (s, 1H), 8.23 (s, 1H), 7.95 (d, J=1.0 Hz, 1H), 7.82 (d, J=8.4 Hz, 2H), 7.55 (d, J=8.2 Hz, 2H), 5.86 (d, J=4.8 Hz, 1H), 5.44 (br d, J=3.2 Hz, 1H), 5.17 (br s, 1H), 4.89-4.73 (m, 2H), 4.68 (br s, 1H), 4.04-3.89 (m, 2H), 1.30 (d, J=6.0 Hz, 3H).

### Compound 329:

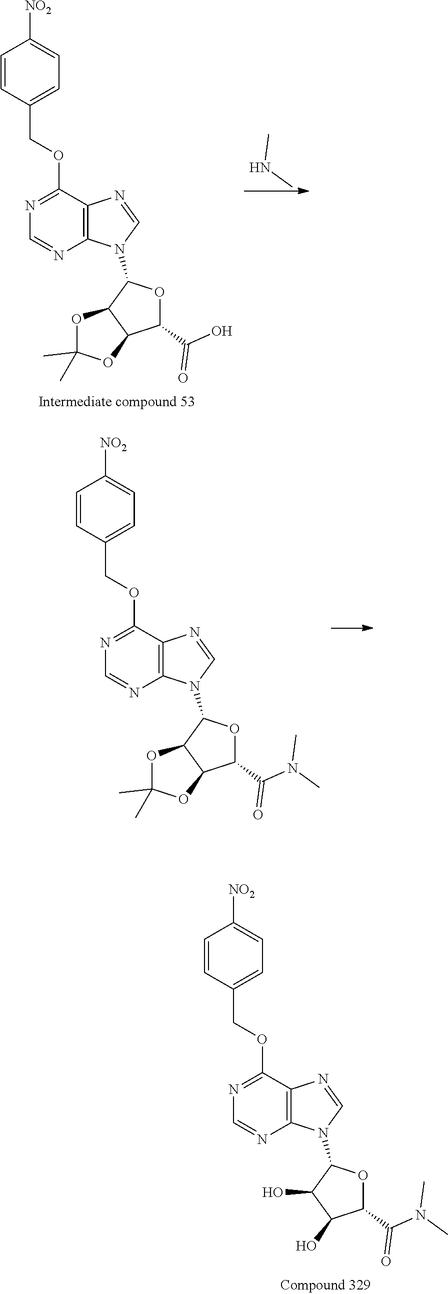

Compound 329 (4.0 mg, 2900 yield) was synthesized from Intermediate Compound 53 and dimethylamine following the protocols described for Compound 271.

LCMS: Rt=0.787 min; (ESI positive ion) m/z: 444.9 (M+H)+(calculated: 445.14).

1H NMR (400 MHz, CDCl₃) δ=8.44 (d, J=8.6 Hz, 2H), 8.25 (br d, J=8.6 Hz, 2H), 7.72 (br d, J=8.4 Hz, 2H), 6.21 (d, J=5.4 Hz, 1H), 5.81-5.69 (m, 2H), 5.47 (br d, J=2.0 Hz, 1H), 5.07 (d, J=1.7 Hz, 1H), 4.73 (br s, 1H), 4.65 (br s, 1H), 3.44 (br s, 1H), 3.15 (s, 3H), 2.99 (s, 3H).

### Compound 330:

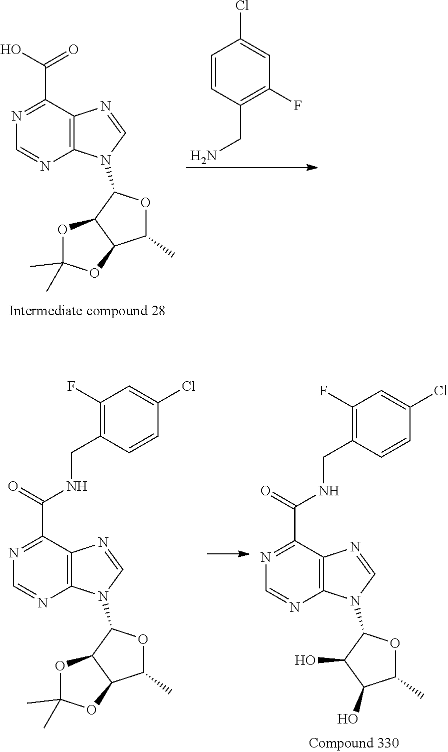 [SMILES: C[C@H]1O[C@@H](n2cnc3c(C(=O)O)ncnc32)[C@@H]2OC(C)(C)O[C@@H]21 ; NCc1ccc(Cl)cc1F ; C[C@H]1O[C@@H](n2cnc3c(C(=O)NCc4ccc(Cl)cc4F)ncnc32)[C@@H]2OC(C)(C)O[C@@H]21 ; C[C@H]1O[C@@H](n2cnc3c(C(=O)NCc4ccc(Cl)cc4F)ncnc32)[C@H](O)[C@@H]1O]

To a solution of Intermediate Compound 28 (100 mg, 312.21 μmol), 1 eq) and (4-chloro-2-fluorophenyl)methanamine (59.79 mg, 374.65 μmol, 1.2 eq) in TH (10 mL) was added T3P (496.69 mg, 780.52 μmol, 464.20 μL, 50% purity, 2.5 eq) and DIEA (201.75 mg, 1.56 mmol, 271.90 μL, 5 eq) at 0° C. The mixture was stirred at 25° C. for 1 hr. The mixture was then filtered and concentrated under reduced pressure to give a residue, which was purified by Prep-TLC (SiO2, Petroleum ether:Ethyl acetate=0:1) to afford the corresponding amide compound (142 mg, 97% yield) as a white solid.

LCMS: Rt=0.580 min; (ESI positive ion) m/z: 462.1 (M+H)+(calculated: 462.13).

A solution of the above amide compound (137 mg, 296.62 μmol, 1 eq) in water (0.8 mL) and formic acid (3.2 mL) was stirred at 0° C. for 6 hr. NH3·H2O was added to the reaction mixture at 0° C. until the pH reached 7. The mixture was then filtered and concentrated under reduced pressure to give a residue. The crude product was dissolved in MeOH (2 mL) and then purified by Prep-HPLC (column: Phenomenex C18 150×25 mm×10 μm; mobile phase: [water (NH4HCO3)-ACN]; B %: 16%-46%, 8 min) and lyophilized to give Compound 330 (30.01 mg, 24% yield) as a white solid.

LCMS: Rt=0.346 min; (ESI positive ion) m/z: 422.0 (M+H)+(calculated: 422.10).

1H NMR (400 MHz, DMSO-d6) δ=9.49 (t, J=6.0 Hz, 1H), 9.07 (s, 1H), 8.95 (s, 1H), 7.52-7.41 (m, 2H), 7.29 (dd, J=1.8, 8.4 Hz, 1H), 6.04 (d, J=4.8 Hz, 1H), 5.53 (d, J=5.6 Hz, 1H), 5.25-5.22 (m, 1H), 4.72 (q, J=5.0 Hz, 1H), 4.60 (d, J=6.2 Hz, 2H), 4.08-3.99 (m, 2H), 1.34 (d, J=6.2 Hz, 3H).

### Compound 335:

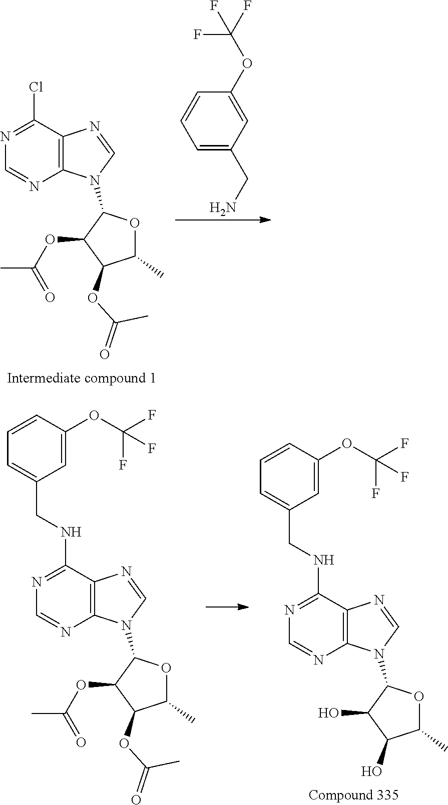 [SMILES: CC(=O)O[C@@H]1[C@H](OC(C)=O)[C@@H](C)O[C@H]1n1cnc2c(Cl)ncnc21 ; NCc1cccc(OC(F)(F)F)c1 ; CC(=O)O[C@@H]1[C@H](OC(C)=O)[C@@H](C)O[C@H]1n1cnc2c(NCc3cccc(OC(F)(F)F)c3)ncnc21 ; C[C@H]1O[C@@H](n2cnc3c(NCc4cccc(OC(F)(F)F)c4)ncnc32)[C@H](O)[C@@H]1O]

Compound 335 (46.28 mg, 5000 yield) was synthesized from Intermediate Compound 1 and (3-(trifluoromethoxy)phenyl)methanamine following the protocols described for Compound 254.

LCMS: Rt=0.801 min; (ESI positive ion) m/z: 426.6 (M+H)+(calculated: 426.13).

1H NMR (400 MHz, DMSO-d6) δ=8.55-8.42 (m, 1H), 8.37 (s, 1H), 8.22 (s, 1H), 7.46-7.40 (m, 1H), 7.39-7.34 (m, 1H), 7.32 (br s, 1H), 7.25-7.18 (m, 1H), 5.85 (d, J=4.8 Hz, 1H), 5.44 (d, J=5.6 Hz, 1H), 5.17 (br d, J=4.8 Hz, 1H), 4.74 (br s, 2H), 4.67 (br d, J=4.0 Hz, 1H), 4.03-3.92 (m, 2H), 1.30 (d, J=6.0 Hz, 3H).

### Compound 336:

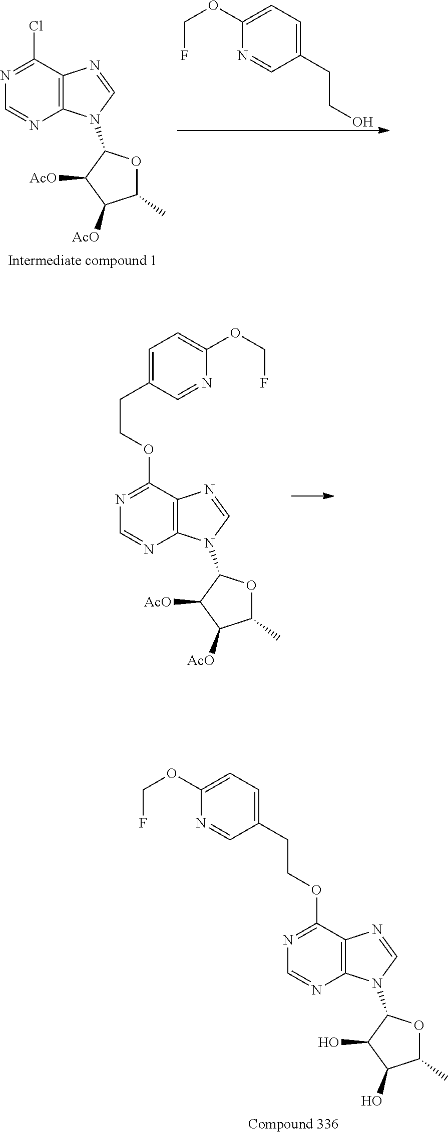 [SMILES: CC(=O)O[C@@H]1[C@H](OC(C)=O)[C@@H](C)O[C@H]1n1cnc2c(Cl)ncnc21 ; OCCc1ccc(OCF)nc1 ; CC(=O)O[C@@H]1[C@H](OC(C)=O)[C@@H](C)O[C@H]1n1cnc2c(OCCc3ccc(OCF)nc3)ncnc21 ; C[C@H]1O[C@@H](n2cnc3c(OCCc4ccc(OCF)nc4)ncnc32)[C@H](O)[C@@H]1O]

Compound 336 (71 mg, 86% yield) was synthesized from Intermediate Compound 1 and 2-(6-(fluoromethoxy)pyridin-3-yl)ethan-1-ol following the protocols described for Compound 188.

LCMS: Rt=0.783 min; (ESI positive ion) m/z: 406.1 (M+H)+(calculated: 406.14).

1H NMR (400 MHz, DMSO-d6) δ=8.57 (s, 1H), 8.54 (s, 1H), 8.19 (d, J=2.1 Hz, 1H), 7.84 (dd, J=2.4, 8.4 Hz, 1H), 6.94 (d, J=8.3 Hz, 1H), 6.13 (s, 1H), 6.00 (s, 1H), 5.93 (d, J=4.9 Hz, 1H), 5.46 (d, J=5.7 Hz, 1H), 5.19 (d, J=5.1 Hz, 1H), 4.76 (t, J=6.6 Hz, 2H), 4.68 (q, J=5.0 Hz, 1H), 4.03-3.96 (m, 2H), 3.14 (t, J=6.5 Hz, 2H), 1.31 (d, J=6.1 Hz, 3H).

### Compound 339:

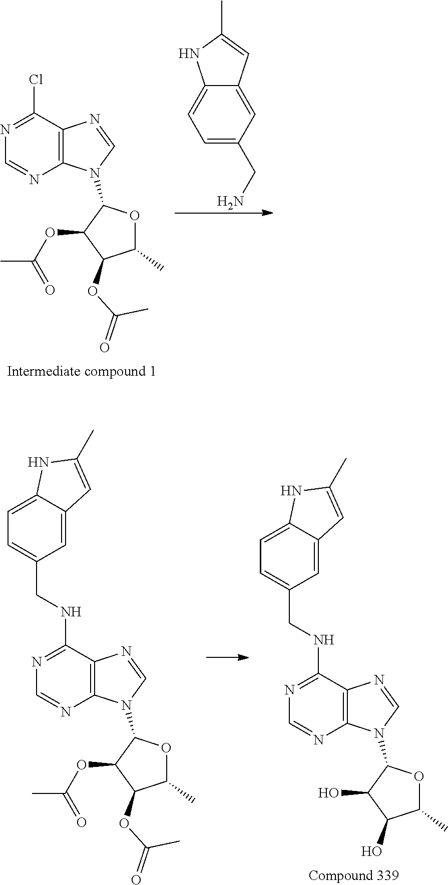 [SMILES: CC(=O)O[C@@H]1[C@H](OC(C)=O)[C@@H](C)O[C@H]1n1cnc2c(Cl)ncnc21 ; Cc1cc2cc(CN)ccc2[nH]1 ; CC(=O)O[C@@H]1[C@H](OC(C)=O)[C@@H](C)O[C@H]1n1cnc2c(NCc3ccc4[nH]c(C)cc4c3)ncnc21 ; Cc1cc2cc(CNc3ncnc4c3ncn4[C@@H]3O[C@H](C)[C@@H](O)[C@H]3O)ccc2[nH]1]

Compound 339 (24.35 mg, 29% yield) was synthesized from Intermediate Compound 1 and (2-methyl-1H-indol-5-yl)methanamine following the protocols described for Compound 254.

LCMS: Rt=0.848 min; (ESI positive ion) m/z: 395.0 (M+H)+(calculated: 395.18).

1H NMR (400 MHz, DMSO-d6) δ=10.79 (br s, 1H), 8.32 (s, 1H), 8.30-8.17 (m, 2H), 7.34 (s, 1H), 7.16 (d, J=8.0 Hz, 1H), 7.00 (dd, J=1.2, 8.4 Hz, 1H), 6.03 (s, 1H), 5.84 (d, J=4.8 Hz, 1H), 5.43 (d, J=5.6 Hz, 1H), 5.15 (d, J=4.8 Hz, 1H), 4.74 (br s, 2H), 4.66 (q, J=4.4 Hz, 1H), 4.03-3.92 (m, 2H), 2.34 (s, 3H), 1.30 (d, J=6.0 Hz, 3H).

### Compound 341:

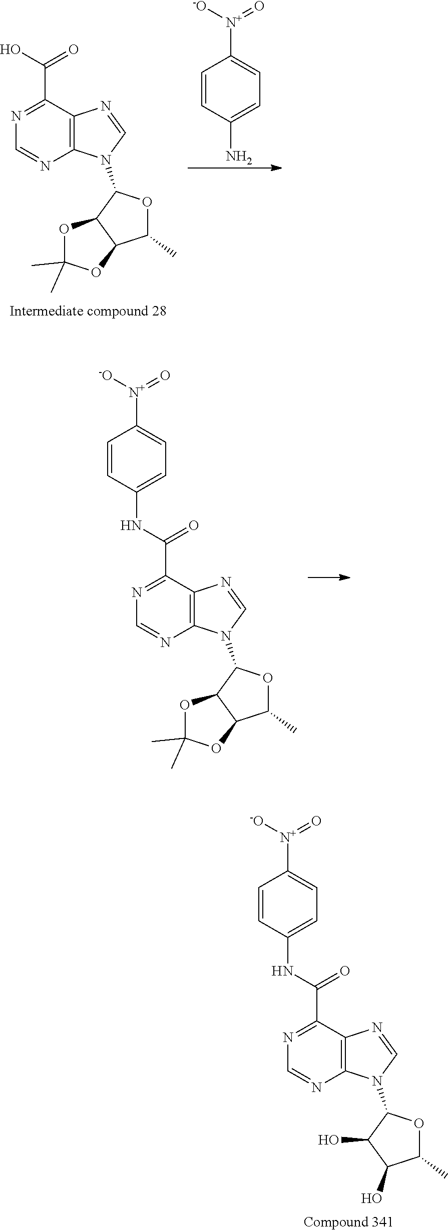

Compound 341 (16.01 mg, 35% yield) was synthesized from Intermediate Compound 28 and 4-nitroaniline following the protocols described for Compound 295.

LCMS: Rt=0.790 min; (ESI positive ion) m/z: 401.0 (M+H)+(calculated: 401.11).

1H NMR (400 MHz, MeOD-d4) δ=9.14 (s, 1H), 8.83 (s, 1H), 8.31 (d, J=9.2 Hz, 2H), 8.15 (d, J=9.2 Hz, 2H), 6.16 (d, J=4.4 Hz, 1H), 4.85 (br s, 1H), 4.21-4.15 (m, 2H), 1.46 (d, J=6.0 Hz, 3H).

### Compound 343:

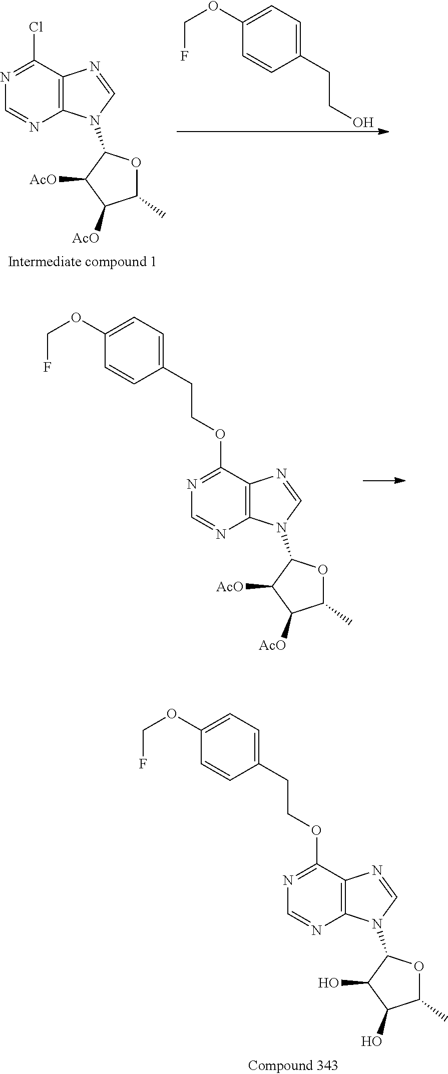 [SMILES: CC(=O)O[C@@H]1[C@H](OC(C)=O)[C@@H](C)O[C@H]1n1cnc2c(Cl)ncnc21 ; OCCc1ccc(OCF)cc1 ; CC(=O)O[C@@H]1[C@H](OC(C)=O)[C@@H](C)O[C@H]1n1cnc2c(OCCc3ccc(OCF)cc3)ncnc21 ; C[C@H]1O[C@@H](n2cnc3c(OCCc4ccc(OCF)cc4)ncnc32)[C@H](O)[C@@H]1O]

Compound 343 (37.7 mg, 28% yield) was synthesized from Intermediate Compound 1 and 2-(4-(fluoromethoxy)phenyl)ethan-1-ol following the protocols described for Compound 188.

LCMS: Rt=0.820 min; (ESI positive ion) m/z: 405.1 (M+H)+(calculated: 405.15).

1H NMR (400 MHz, DMSO-d6) δ=8.58 (s, 1H), 8.54 (s, 1H), 7.33 (d, J=8.6 Hz, 2H), 7.05 (d, J=8.4 Hz, 2H), 5.93 (d, J=5.0 Hz, 1H), 5.90 (s, 1H), 5.76 (s, 1H), 5.55 (br s, 1H), 5.28 (br s, 1H), 4.73 (t, J=6.8 Hz, 2H), 4.68 (br s, 1H), 3.99 (br d, J=4.6 Hz, 2H), 3.11 (t, J=6.8 Hz, 2H), 1.31 (br d, J=6.0 Hz, 3H).

### Compound 344:

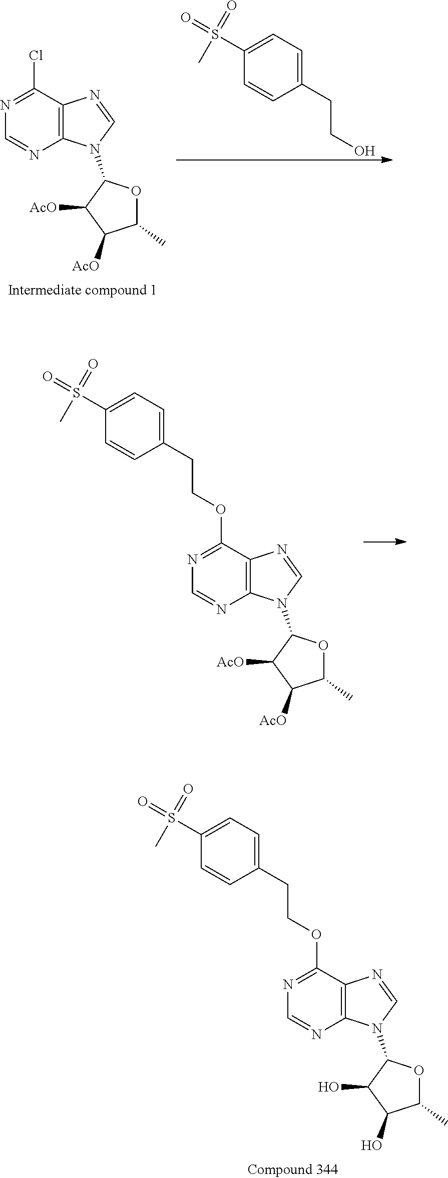 [SMILES: CC(=O)O[C@@H]1[C@H](OC(C)=O)[C@@H](C)O[C@H]1n1cnc2c(Cl)ncnc21 ; CS(=O)(=O)c1ccc(CCO)cc1 ; CC(=O)O[C@@H]1[C@H](OC(C)=O)[C@@H](C)O[C@H]1n1cnc2c(OCCc3ccc(S(C)(=O)=O)cc3)ncnc21 ; C[C@H]1O[C@@H](n2cnc3c(OCCc4ccc(S(C)(=O)=O)cc4)ncnc32)[C@H](O)[C@@H]1O]

Compound 344 (27.34 mg, 25% yield) was synthesized from Intermediate Compound 1 and 2-(4-(methylsulfonyl)phenyl)ethan-1-ol following the protocols described for Compound 188.

LCMS: Rt=0.764 min; (ESI positive ion) m/z: 435.2 (M+H)+(calculated: 435.13).

1H NMR (400 MHz, DMSO-d6) δ=8.56 (d, J=8.0 Hz, 2H), 7.87 (d, J=8.4 Hz, 2H), 7.62 (d, J=8.4 Hz, 2H), 5.93 (d, J=5.2 Hz, 1H), 5.52 (br s, 1H), 5.24 (br s, 1H), 4.83 (t, J=6.4 Hz, 2H), 4.67 (br s, 1H), 4.02-3.96 (m, 2H), 3.27 (br t, J=6.8 Hz, 2H), 3.19 (s, 3H), 1.31 (d, J=6.0 Hz, 3H).

### Compound 345:

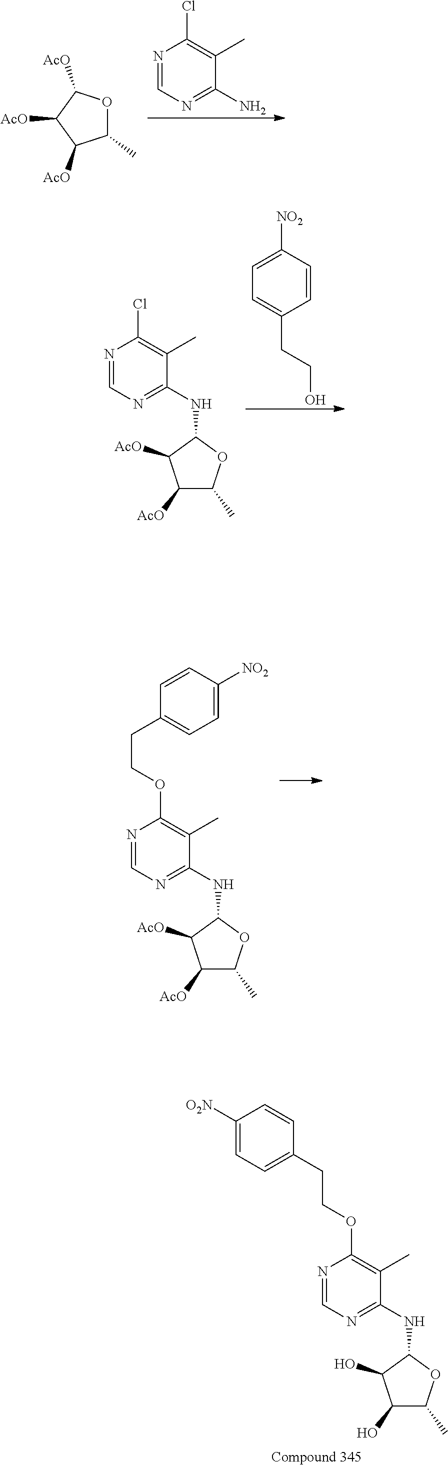

To a solution of 6-chloro-5-methylpyrimidin-4-amine (662.04 mg, 4.61 mmol, 1.2 eq) in MeCN (10 mL) was added bis(trimethylsilyl)acetamide (1.56 g, 7.69 mmol, 1.90 mL, 2 eq). The mixture was stirred at 80° C. for 2 hr. After 2 hr, the mixture was concentrated to give a residue. Then a solution of (2S,3R,4R,5R)-5-methyltetrahydrofuran-2,3,4-triyl triacetate (1 g, 3.84 mmol, 1 eq) in MeCN (10 mL) was added to the residue and tin(IV) chloride (3.00 g, 11.53 mmol, 1.35 mL, 3 eq) was added to the mixture dropwise at 0° C. The mixture was stirred at 20° C. for 12 hr. The reaction mixture was then poured into a saturated aqueous solution of NaHCO3 (50 mL) and extracted with ethyl acetate (50 mL×3). The organic layer was washed with brine, dried by Na2SO4 and concentrated to give a residue, which was purified by column chromatography (SiO2, Petroleum ether:Ethyl acetate=1:0 to 0:1) to give the corresponding amine compound (1 g) as a brown solid.

LCMS: Rt=0.825 min; (ESI positive ion) m/z: 344.0 (M+H)+(calculated: 344.09).

A mixture of the above amine compound (200 mg, 581.80 μmol, 1 eq), 2-(4-nitrophenyl)ethan-1-ol (126.43 mg, 756.34 μmol, 1.3 eq), Cs2CO3 (473.90 mg, 1.45 mmol, 2.5 eq), and RuPhos Pd G4 (49.48 mg, 58.18 μmol, 0.1 eq) in dioxane (2 mL) was degassed and purged with N2 3 times. Then the mixture was stirred at 80° C. for 16 hr. The reaction mixture was then concentrated to give the crude product, which was purified by column chromatography (SiO2, Petroleum ether/Ethyl acetate=10/1 to 1/1) to give the corresponding ether compound (197 mg) as a yellow oil.

LCMS: Rt=0.948 min; (ESI positive ion) m/z: 475.1 (M+H)+(calculated: 475.18).

To a solution of the above ether compound (197 mg, 415.21 μmol, 1 eq) in MeOH (3 mL) was added NH3·H2O (2.24 g, 17.90 mmol, 2.46 mL, 28% purity, 43.12 eq). The mixture was stirred at 25° C. for 1 hr. The reaction mixture was then filtered and concentrated to give a residue, which was purified by Prep-HPLC (column: Unisil 3-100 C18 Ultra 150×50 mm×3 μm; mobile phase: [water(FA)-ACN]; B %: 20%-50%, 10 min). The residue was further separated by SFC (column: Daicel ChiralPak IG (250×30 mm, 10 um); mobile phase: [0.1% NH3·H2O ETOH]; B %: 40%-40%, 6.4; 60 min) to give Compound 345 (8.9 mg) as a white solid.

LCMS: Rt=0.814 min; (ESI positive ion) m/z: 391.0 (M+H)+(calculated: 391.15).

SFC: Rt=0.597 min, 51.3% e.e. value (Column: Chiralpak IG-3 50×4.6 mm I.D., 3 μm; Mobile phase: Phase A for CO2, and Phase B for EtOH (0.05% DEA); Gradient elution: 40% EtOH (0.05% DEA) in CO2; Flow rate: 3 mL/min; Detector: PDA; Column Temp: 35 C; Back Pressure: 100 Bar)

1H NMR (400 MHz, MeOD-d4) δ=8.18 (d, J=8.8 Hz, 2H), 8.11 (d, J=3.4 Hz, 1H), 7.52 (d, J=8.8 Hz, 2H), 6.06-5.62 (m, 1H), 4.61-4.56 (m, 2H), 4.21-4.10 (m, 1H), 3.99-3.72 (m, 2H), 1.93-1.86 (m, 3H), 1.30-1.21 (m, 3H).

### Compound 347:

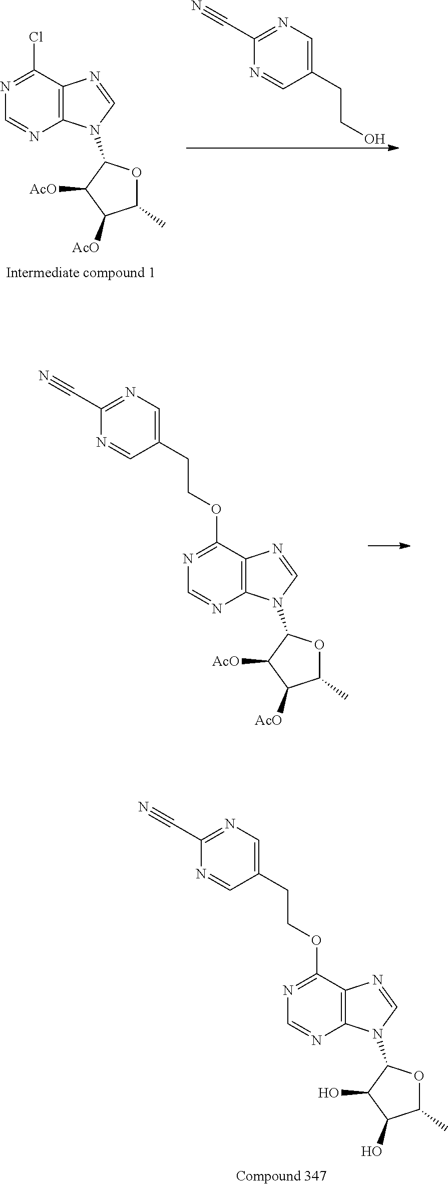 [SMILES: CC(=O)O[C@@H]1[C@H](OC(C)=O)[C@@H](C)O[C@H]1n1cnc2c(Cl)ncnc21 ; N#Cc1ncc(CCO)cn1 ; CC(=O)O[C@@H]1[C@H](OC(C)=O)[C@@H](C)O[C@H]1n1cnc2c(OCCc3cnc(C#N)nc3)ncnc21 ; C[C@H]1O[C@@H](n2cnc3c(OCCc4cnc(C#N)nc4)ncnc32)[C@H](O)[C@@H]1O]

Compound 347 (5.29 mg, 6% yield) was synthesized from Intermediate Compound 1 and 5-(2-hydroxyethyl)pyrimidine-2-carbonitrile following the protocols described for Compound 188.

LCMS: Rt=0.719 min; (ESI positive ion) m/z: 384.1 (M+H)+(calculated: 384.13).

1H NMR (400 MHz, DMSO-d6) δ=9.03 (s, 2H), 8.58 (s, 1H), 8.53 (s, 1H), 5.93 (d, J=5.0 Hz, 1H), 5.48 (d, J=5.6 Hz, 1H), 5.22 (br d, J=3.8 Hz, 1H), 4.86 (t, J=6.4 Hz, 2H), 4.67 (q, J=4.8 Hz, 1H), 4.02-3.97 (m, 2H), 3.30-3.29 (m, 2H), 1.31 (d, J=6.0 Hz, 3H).

### Compound 349:

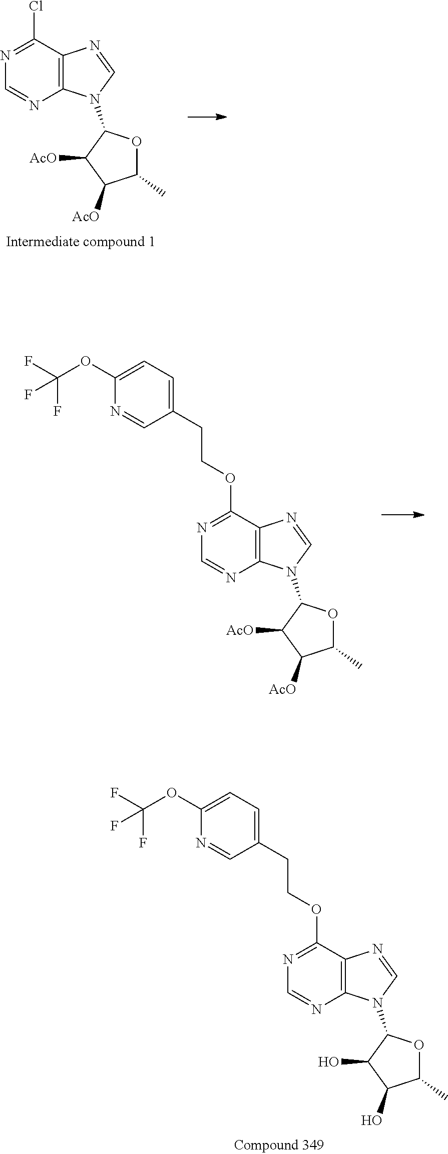 [SMILES: CC(=O)O[C@@H]1[C@H](OC(C)=O)[C@@H](C)O[C@H]1n1cnc2c(Cl)ncnc21 ; CC(=O)O[C@@H]1[C@H](OC(C)=O)[C@@H](C)O[C@H]1n1cnc2c(OCCc3ccc(OC(F)(F)F)nc3)ncnc21 ; C[C@H]1O[C@@H](n2cnc3c(OCCc4ccc(OC(F)(F)F)nc4)ncnc32)[C@H](O)[C@@H]1O]

Compound 349 (48.54 mg, 41% yield) was synthesized from Intermediate Compound 1 and 2-(6-(trifluoromethoxy)pyridin-3-yl)ethan-1-ol following the protocols described for Compound 188.

LCMS: Rt=0.838 min; (ESI positive ion) m/z: 442.0 (M+H)+(calculated: 442.13).

1H NMR (400 MHz, DMSO-d6) δ=8.55 (d, J=14.2 Hz, 2H), 8.34 (d, J=2.2 Hz, 1H), 8.02 (dd, J=2.4, 8.4 Hz, 1H), 7.25 (d, J=8.3 Hz, 1H), 5.93 (d, J=5.0 Hz, 1H), 5.47 (d, J=5.7 Hz, 1H), 5.20 (d, J=5.1 Hz, 1H), 4.80 (t, J=6.5 Hz, 2H), 4.67 (q, J=5.0 Hz, 1H), 4.03-3.96 (m, 2H), 3.21 (t, J=6.5 Hz, 2H), 1.31 (d, J=6.1 Hz, 3H).

### Compound 350:

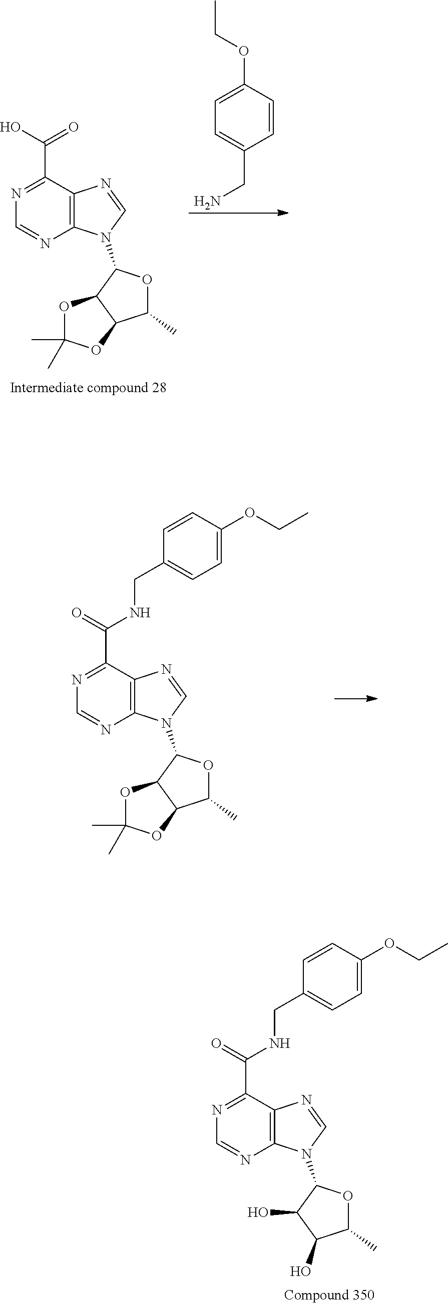 [SMILES: C[C@H]1O[C@@H](n2cnc3c(C(=O)O)ncnc32)[C@@H]2OC(C)(C)O[C@@H]21 ; CCOc1ccc(CN)cc1 ; CCOc1ccc(CNC(=O)c2ncnc3c2ncn3[C@@H]2O[C@H](C)[C@H]3OC(C)(C)O[C@H]32)cc1 ; CCOc1ccc(CNC(=O)c2ncnc3c2ncn3[C@@H]2O[C@H](C)[C@@H](O)[C@H]2O)cc1]

To a solution of Intermediate Compound 28 (100 mg, 312.21 μmol, 1 eq) and (4-ethoxyphenyl)methanamine (56.65 mg, 374.65 μmol, 1.2 eq) in THE (5 mL) was added DIEA (201.75 mg, 1.56 mmol, 271.90 μL, 5 eq) and T3P (496.69 mg, 780.52 μmol, 464.20 μL, 50% purity, 2.5 eq) at 0° C. Then the mixture was stirred at 20° C. for 2 hr. The reaction mixture filtered and the filtrate was concentrated under reduced pressure to give a residue, which was purified by Prep-TLC (SiO2, PE:EtOAc=0:1) to afford the corresponding amide compound (129 mg, 91% yield) as a yellow oil.

LCMS: Rt=0.512 min; (ESI positive ion) m/z: 454.2 (M+H)+(calculated: 454.20).

To a solution of above amide compound (100 mg, 220.51 μmol, 1 eq) was added formic acid/water (4:1 V/V) (4 mL) at 0° C. The reaction was stirred at 0° C. for 6 hrs. NH3·H2O was then added to the reaction mixture at 0° C. until the pH reached 7. The mixture was then filtered and concentrated under reduced pressure to give a residue, which was purified by Prep-HPLC (column: Phenomenex C18 150×25 mm×10 μm; mobile phase: [water (NH4HCO3) -ACN]; B %: 15%-45%, 8 min) and lyophilized to afford Compound 350 (21.37 mg, 22% yield) as a white solid.

LCMS: Rt=0.331 min; (ESI positive ion) m/z: 414.2 (M+H)+(calculated: 414.17).

1H NMR (400 MHz, DMSO-d6), δ=9.39-9.34 (m, 1H), 9.06-9.04 (m, 1H), 8.93 (s, 1H), 7.32-7.28 (m, 2H), 6.90-6.87 (m, 2H), 6.03 (d, J=5.0 Hz, 1H), 5.62-5.42 (m, 1H), 5.24 (br d, J=1.6 Hz, 1H), 4.71 (t, J=4.8 Hz, 1H), 4.50 (d, J=6.2 Hz, 2H), 4.04-3.97 (m, 4H), 1.35-1.29 (m, 6H).

### Compound 351:

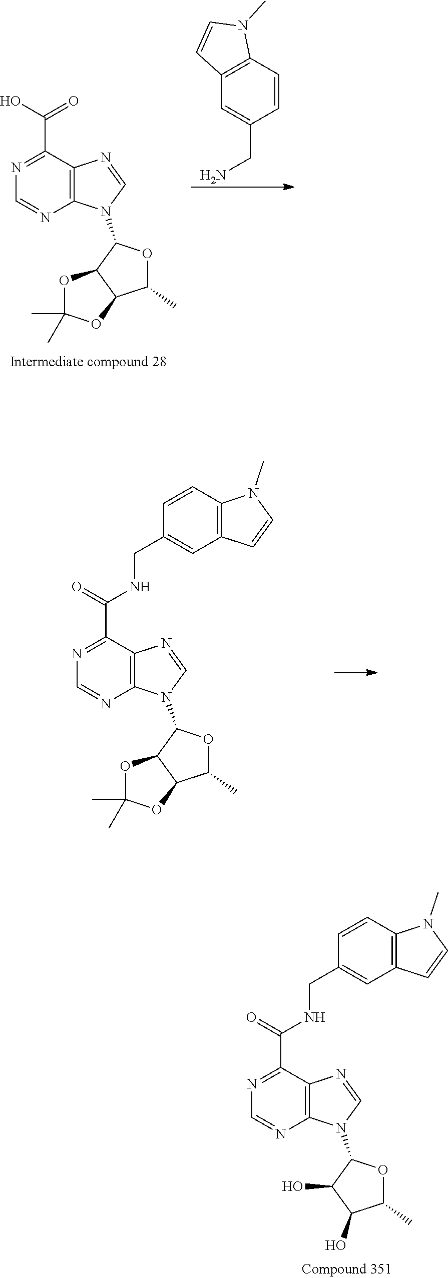 [SMILES: C[C@H]1O[C@@H](n2cnc3c(C(=O)O)ncnc32)[C@@H]2OC(C)(C)O[C@@H]21 ; Cn1ccc2cc(CN)ccc21 ; C[C@H]1O[C@@H](n2cnc3c(C(=O)NCc4ccc5c(ccn5C)c4)ncnc32)[C@@H]2OC(C)(C)O[C@@H]21 ; C[C@H]1O[C@@H](n2cnc3c(C(=O)NCc4ccc5c(ccn5C)c4)ncnc32)[C@H](O)[C@@H]1O]

To a solution of Intermediate Compound 28 (100 mg, 312.21 μmol, 1 eq), (1-methyl-1H-indol-5-yl)methanamine (60.02 mg, 374.65 μmol, 1.2 eq) in THE (10 mL) was added T3P (496.69 mg, 780.52 μmol, 464.20 μL, 50% purity, 2.5 eq) and DIEA (201.75 mg, 1.56 mmol, 271.90 μL, 5 eq) at 0° C. The mixture was stirred at 25° C. for 1 hr. The mixture was then filtered and concentrated under reduced pressure to give a residue, which was purified by Prep-TLC (SiO2, Petroleum ether:Ethyl acetate=0:1) to afford the corresponding amide compound (121 mg, 80% yield) as a white solid.

LCMS: Rt=0.427 min; (ESI positive ion) m/z: 463.2 (M+H)+(calculated: 463.20).

A solution of the above amide compound (116 mg, 250.81 μmol, 1 eq) in formic acid (4 mL) and water (1 mL) was stirred at 0° C. for 6 hr. NH3·H2O was added to the reaction mixture at 0° C. until the pH reached 7. The mixture was then filtered and concentrated under reduced pressure to give a residue. The residue was dissolved in MeOH (2 mL) and then purified by Prep-HPLC (column: Phenomenex C18 150×25 mm×10 μm; mobile phase: [column: Phenomenex C18 150×25 mm×10 μm; mobile phase: [water (NH4HCO3)-ACN]; B %: 12%-42%, 8 min) to afford Compound 351 (3.41 mg, 5% yield) as a white solid.

LCMS: Rt=0.334 min; (ESI positive ion) m/z: 423.2 (M+H)+(calculated: 423.17).

1H NMR (400 MHz, DMSO-d6) δ=9.39 (br t, J=5.8 Hz, 1H), 9.06 (s, 1H), 8.93 (s, 1H), 7.56 (s, 1H), 7.40 (d, J=8.2 Hz, 1H), 7.30 (d, J=3.0 Hz, 1H), 7.23-7.18 (m, 1H), 6.38 (d, J=2.4 Hz, 1H), 6.03 (d, J=4.8 Hz, 1H), 5.54 (d, J=5.8 Hz, 1H), 5.24 (d, J=4.8 Hz, 1H), 4.73-4.68 (m, 1H), 4.74-4.63 (m, 1H), 4.06-3.99 (m, 2H), 3.77 (s, 3H), 1.33 (br d, J=6.0 Hz, 3H).

### Compound 353:

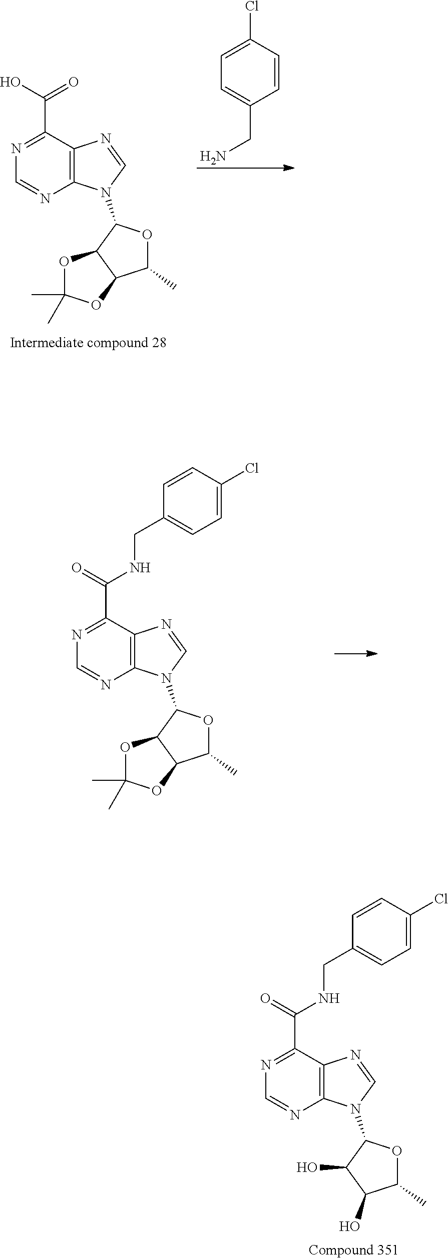 [SMILES: C[C@H]1O[C@@H](n2cnc3c(C(=O)O)ncnc32)[C@@H]2OC(C)(C)O[C@@H]21 ; NCc1ccc(Cl)cc1 ; C[C@H]1O[C@@H](n2cnc3c(C(=O)NCc4ccc(Cl)cc4)ncnc32)[C@@H]2OC(C)(C)O[C@@H]21 ; C[C@H]1O[C@@H](n2cnc3c(C(=O)NCc4ccc(Cl)cc4)ncnc32)[C@H](O)[C@@H]1O]

To a solution of Intermediate Compound 28 (100 mg, 312.21 μmol, 1 eq) and (4-chlorophenyl)methanamine (53.05 mg, 374.65 μmol, 45.73 μL, 1.2 eq) in THE (5 mL) was added T3P (496.69 mg, 780.52 μmol, 464.20 μL, 50% purity, 2.5 eq) and DIEA (201.75 mg, 1.56 mmol, 271.90 μL, 5 eq) at 0° C. The mixture was stirred at 20° C. for 2 hr. The solvent was removed under reduced pressure to give a residue, which was purified by Prep-TLC (SiO2, Petroleum ether:ethyl acetate=0:1) to afford the corresponding amide compound (0.133 g, 96% yield) as a red gum. LCMS: Rt=0.569 min; (ESI positive ion) m/z: 444.2 (M+H)+(calculated: 444.14).

A solution of the above amide compound (120 mg, 270.34 μmol, 1 eq) in formic acid/water (4/1 V/V) (4 mL) was stirred at 0° C. for 8 hr. NH3·H2O was added to the reaction mixture at 0° C. until a pH of 7 was achieved. The mixture was then diluted with H2O (20 ml×3) and extracted with ethyl acetate mL (20 mL×3). The combined organic layers were concentrated under reduced pressure to give a residue, which was purified by Prep-HPLC (column: Waters xbridge 150×25 mm 10 μm; mobile phase: [water (NH4HCO3)-ACN]; B %: 17%-47%, 8 min) to afford Compound 353 (38.46 mg, 34% yield) as a white solid.

LCMS: Rt=0.429 min; (ESI positive ion) m/z: 404.0 (M+H)+(calculated: 404.10).

1H NMR (400 MHz, DMSO-d6) δ=9.50 (t, J=6.0 Hz, 1H), 9.06 (s, 1H), 8.94 (s, 1H), 7.41-7.39 (m, 4H), 6.03 (d, J=4.9 Hz, 1H), 5.52 (d, J=5.6 Hz, 1H), 5.26-5.19 (m, 1H), 4.72 (q, J=5.1 Hz, 1H), 4.58-4.54 (m, 2H), 4.08 (s, 2H), 1.34 (d, J=6.1 Hz, 3H).

### Compound 354:

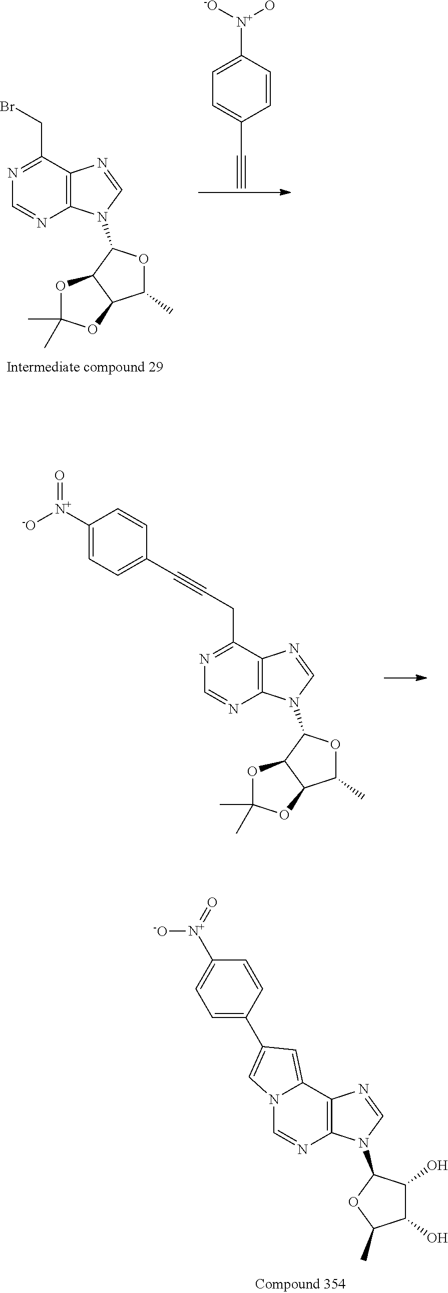

To a solution of Intermediate Compound 29 (100 mg, 270.85 μmol, 1 eq) and 1-ethynyl-4-nitrobenzene (99.62 mg, 677.12 μmol, 2.5 eq) in MeCN (2 mL) was added Xphos Pd G4 (23.31 mg, 27.08 μmol, 0.1 eq) and Cs2CO3 (264.74 mg, 812.54 μmol, 3 eq). The mixture was stirred at 90° C. for 1 hr. The mixture was then concentrated under vacuum and the residue was purified by Prep-TLC (SiO2, Petroleum ether:Ethyl acetate=1:1) to afford the corresponding acetylenylated compound (40 mg, 31% yield) as a yellow solid.

LCMS: Rt=0.422 min; (ESI positive ion) m/z: 436.2 (M+H)+(calculated: 436.15).

A solution of the above acetylenylated compound (58 mg, 133.20 μmol, 1 eq) in formic acid (2 mL) and water (0.5 mL) was stirred at 25° C. for 1 hr. NH3·H2O was added to the reaction mixture at 0° C. until the pH reached 7. The mixture was then filtered and concentrated under reduced pressure to give a residue, which was purified by Prep-HPLC (column: Phenomenex luna C18 150×25 mm×10 μm; mobile phase: [water(FA)-ACN]; B %: 25%-55%, 10 min) to afford Compound 354 (5 mg, 10% yield) as a yellow solid.

LCMS: Rt=0.363 min; (ESI positive ion) m/z: 396.0 (M+H)+(calculated: 496.12).

1H NMR (400 MHz, DMSO-d6) δ=9.21 (s, 1H), 8.40 (s, 1H), 8.33 (d, J=8.8 Hz, 2H), 8.00 (d, J=8.8 Hz, 2H), 7.27 (d, J=4.0 Hz, 1H), 6.82 (d, J=4.0 Hz, 1H), 5.93 (d, J=5.2 Hz, 1H), 5.50 (d, J=5.2 Hz, 1H), 5.20 (d, J=5.2 Hz, 1H), 4.69-4.60 (m, 1H), 4.10-3.95 (m, 2H), 1.33 (d, J=6.0 Hz, 3H)

### Compound 356:

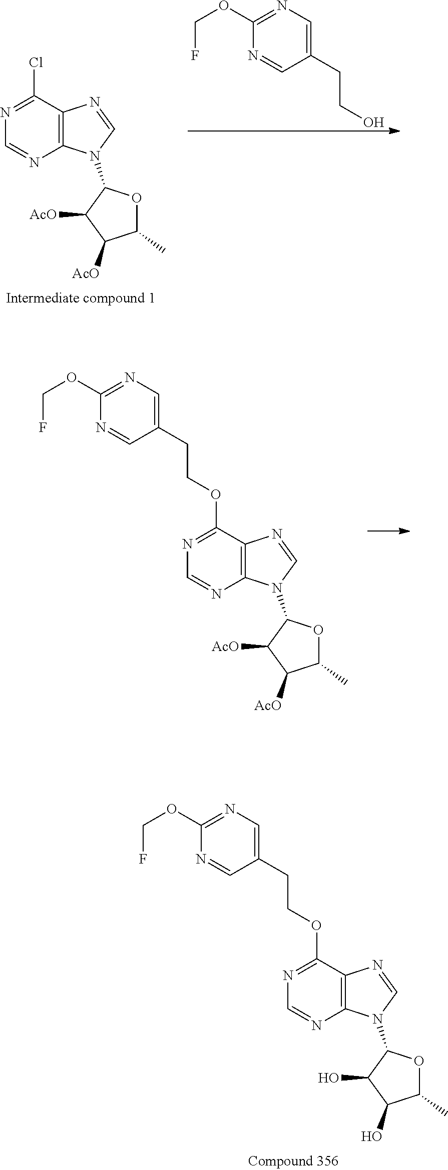 [SMILES: CC(=O)O[C@@H]1[C@H](OC(C)=O)[C@@H](C)O[C@H]1n1cnc2c(Cl)ncnc21 ; OCCc1cnc(OCF)nc1 ; CC(=O)O[C@@H]1[C@H](OC(C)=O)[C@@H](C)O[C@H]1n1cnc2c(OCCc3cnc(OCF)nc3)ncnc21 ; C[C@H]1O[C@@H](n2cnc3c(OCCc4cnc(OCF)nc4)ncnc32)[C@H](O)[C@@H]1O]

Compound 356 (17.22 mg, 34% yield) was synthesized from Intermediate Compound 1 and 2-(2-(fluoromethoxy)pyrimidin-5-yl)ethan-1-ol following the protocols described for Compound 188.

LCMS: Rt=0.723 min; (ESI positive ion) m/z: 407.0 (M+H)+(calculated: 407.14).

1H NMR (400 MHz, DMSO-d6) δ=8.69 (s, 2H), 8.56 (d, J=16.9 Hz, 2H), 6.15 (s, 1H), 6.02 (s, 1H), 5.93 (d, J=5.0 Hz, 1H), 5.47 (d, J=5.6 Hz, 1H), 5.20 (br d, J=5.0 Hz, 1H), 4.79 (t, J=6.3 Hz, 2H), 4.67 (q, J=5.0 Hz, 1H), 4.04-3.96 (m, 2H), 3.16 (t, J=6.4 Hz, 2H), 1.31 (d, J=6.1 Hz, 3H).

### Compound 357:

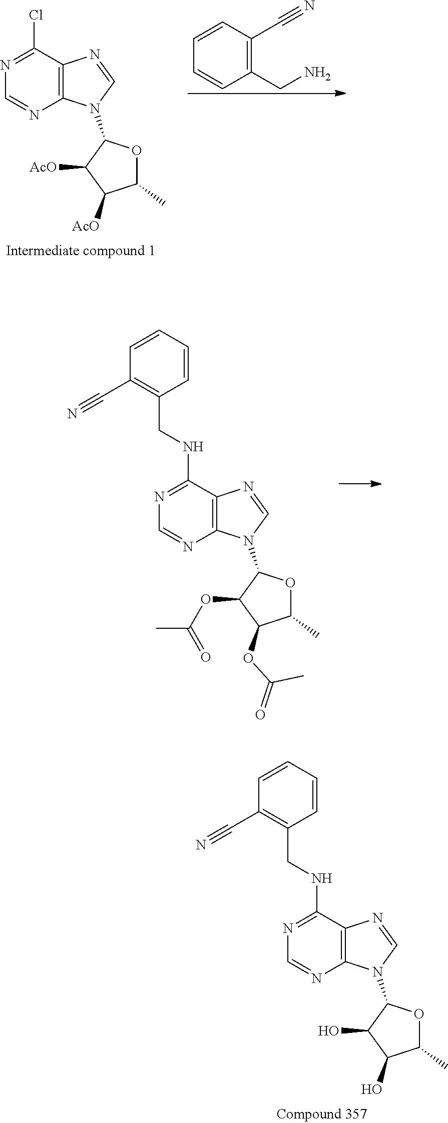 [SMILES: CC(=O)O[C@@H]1[C@H](OC(C)=O)[C@@H](C)O[C@H]1n1cnc2c(Cl)ncnc21 ; N#Cc1ccccc1CN ; CC(=O)O[C@@H]1[C@H](OC(C)=O)[C@@H](C)O[C@H]1n1cnc2c(NCc3ccccc3C#N)ncnc21 ; C[C@H]1O[C@@H](n2cnc3c(NCc4ccccc4C#N)ncnc32)[C@H](O)[C@@H]1O]

Compound 357 (13.63 mg, 7% yield) was synthesized from Intermediate Compound 1 and 2-(aminomethyl)benzonitrile following the protocols described for Compound 254.

LCMS: Rt=0.673 min; (ESI positive ion) m/z: 367.0 (M+H)+(calculated: 367.14).

1H NMR (400 MHz, DMSO-d6) δ=8.71 (s, 1H), 8.68 (s, 1H), 8.25 (s, 1H), 7.88 (d, J=7.2 Hz, 1H), 7.73-7.68 (m, 1H), 7.63 (dt, J=0.8, 7.2 Hz, 1H), 7.56-7.48 (m, 1H), 6.00 (d, J=4.8 Hz, 1H), 5.59 (s, 2H), 4.72 (t, J=4.8 Hz, 1H), 4.07-3.98 (m, 2H), 1.37-1.31 (m, 3H).

### Compound 360:

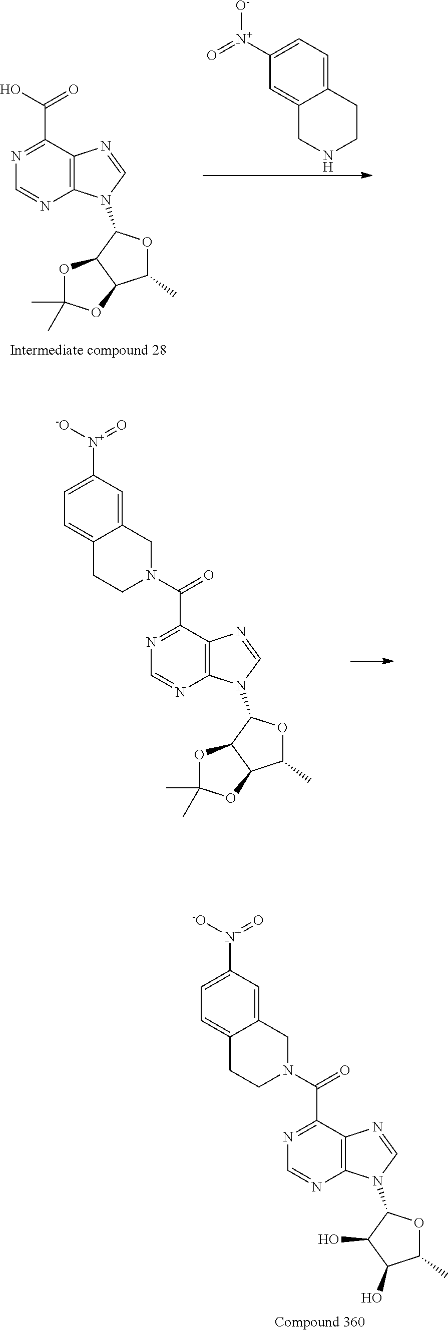

To a solution of Intermediate Compound 28 (120 mg, 374.65 μmol, 1 eq) and 7-nitro-1,2,3,4-tetrahydroisoquinoline (80.11 mg, 449.58 μmol, 1.2 eq) in THE (10 mL) was added T3P (596.03 mg, 936.62 μmol, 557.04 μL, 50% purity, 2.5 eq) and DIEA (242.10 mg, 1.87 mmol, 326.29 μL, 5 eq) at 0° C. The mixture was stirred at 25° C. for 1 hr. The mixture was then filtered and concentrated under reduced pressure to give a residue, which was purified by Prep-TLC (SiO2, Petroleum ether:Ethyl acetate=0:1) to afford the corresponding amide compound (67 mg, 35% yield) as a yellow solid.

LCMS: Rt=0.529 min; (ESI positive ion) m/z: 481.2 (M+H)+(calculated: 481.18).

A solution of the above amide compound (67 mg, 139.45 μmol, 1 eq) in water (0.8 mL) and formic acid (3.2 mL) was stirred at 25° C. for 1 hr. NH3·H2O was added to the reaction mixture at 0° C. until the pH reached 7. The mixture was then filtered and concentrated under reduced pressure to give a residue. The crude product was dissolved in MeOH (2 mL) and then purified by Prep-HPLC (column: Waters xbridge 150×25 mm 10 μm; mobile phase: [water (NH4HCO3)-ACN]; B %: 10%-40%, 11 min) to afford Compound 360 (23 mg, 37% yield) as a white solid.

LCMS: Rt=0.311 min; (ESI positive ion) m/z: 441.1 (M+H)+(calculated: 441.14).

1H NMR (400 MHz, DMSO-d6) δ=9.02 (br d, J=11.0 Hz, 1H), 8.86 (br d, J=16.0 Hz, 1H), 8.29 (br s, 1H), 8.11-8.01 (m, 1H), 7.93 (br s, 1H), 7.49 (br dd, J=8.6, 15.5 Hz, 1H), 6.03 (br d, J=4.4 Hz, 1H), 5.56-5.52 (m, 1H), 5.27 (br d, J=3.8 Hz, 1H), 5.05 (br s, 1H), 4.74 (br d, J=4.4 Hz, 1H), 4.60 (s, 1H), 4.04 (br s, 2H), 3.50 (br d, J=5.0 Hz, 1H), 3.09 (br s, 1H), 2.96-2.92 (m, 1H), 1.35 (br d, J=5.2 Hz, 3H).

### Compound 361:

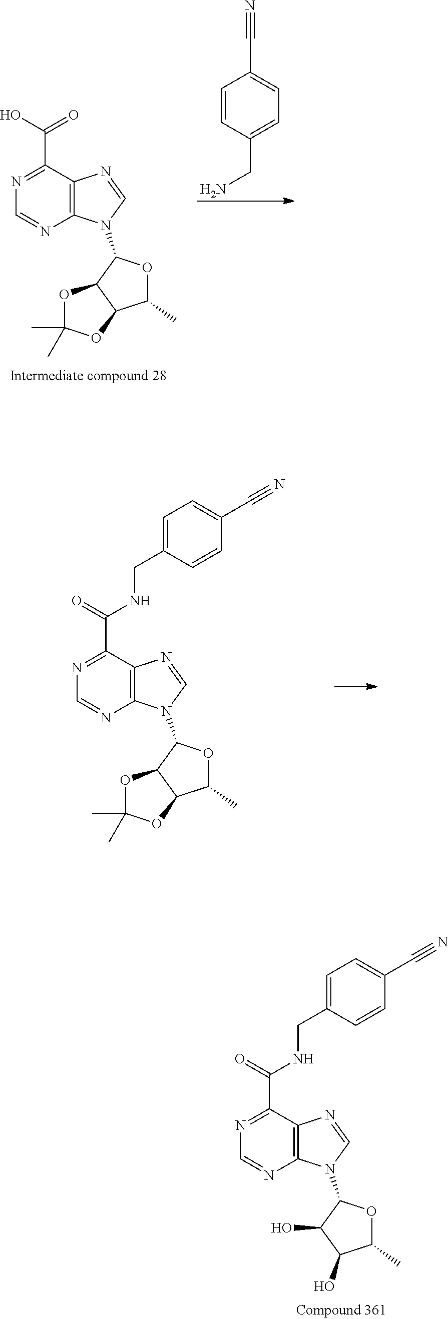 [SMILES: C[C@H]1O[C@@H](n2cnc3c(C(=O)O)ncnc32)[C@@H]2OC(C)(C)O[C@@H]21 ; N#Cc1ccc(CN)cc1 ; C[C@H]1O[C@@H](n2cnc3c(C(=O)NCc4ccc(C#N)cc4)ncnc32)[C@@H]2OC(C)(C)O[C@@H]21 ; C[C@H]1O[C@@H](n2cnc3c(C(=O)NCc4ccc(C#N)cc4)ncnc32)[C@H](O)[C@@H]1O]

To a solution of Intermediate Compound 28 (100 mg, 312.21 μmol, 1 eq) and 4-(aminomethyl)benzonitrile (49.51 mg, 374.65 μmol, 1.2 eq) in THF (10 mL) was added DIEA (201.75 mg, 1.56 mmol, 271.90 μL) and T3P (496.69 mg, 780.52 μmol, 464.20 μL, 50% purity) at 0° C. Then the mixture was stirred at 25° C. for 12 hr. The reaction mixture was filtered and filtrate was concentrated under reduced pressure to give a residue, which was purified by {rep-TLC (SiO2, PE:EA=0:1) and concentrated under vacuum to afford the corresponding amide compound (133 mg, 98% yield) as a yellow solid.

LCMS: Rt=0.494 min; (ESI positive ion) m/z: 435.2 (M+H)+(calculated: 435.17).

To a solution of the above amide compound (100 mg, 230.18 μmol, 1 eq) was added formic acid/water (4/1 V/V) (10 mL) at 0° C. The reaction was stirred at 0° C. for 12 hr. NH3·H2O was then added to the reaction mixture at 0° C. until the pH reached 7. The mixture was then filtered and concentrated under reduced pressure to give a residue, which was purified by Prep-HPLC (column: Phenomenex C18 150×25 mm×10 μm; mobile phase: [water (NH4HCO3)-ACN]; B %: 7%-37%, 8 min) to afford Compound 361 (18.11 mg, 20% yield) as a white solid.

LCMS: Rt=0.302 min; (ESI positive ion) m/z: 395.1 (M+H)+(calculated: 395.14).

1H NMR (400 MHz, DMSO-d6), δ=9.61-9.57 (m, 1H), 9.07 (s, 1H), 8.96-8.94 (m, 1H), 7.82 (d, J=8.4 Hz, 2H), 7.58 (d, J=8.4 Hz, 2H), 6.05-6.03 (m, 1H), 5.53 (d, J=5.6 Hz, 1H), 5.24 (d, J=5.2 Hz, 1H), 4.74-4.70 (m, 1H), 4.66 (d, J=6.2 Hz, 2H), 4.03 (br t, J=5.4 Hz, 2H), 1.34 (d, J=6.0 Hz, 3H).

### Compound 368:

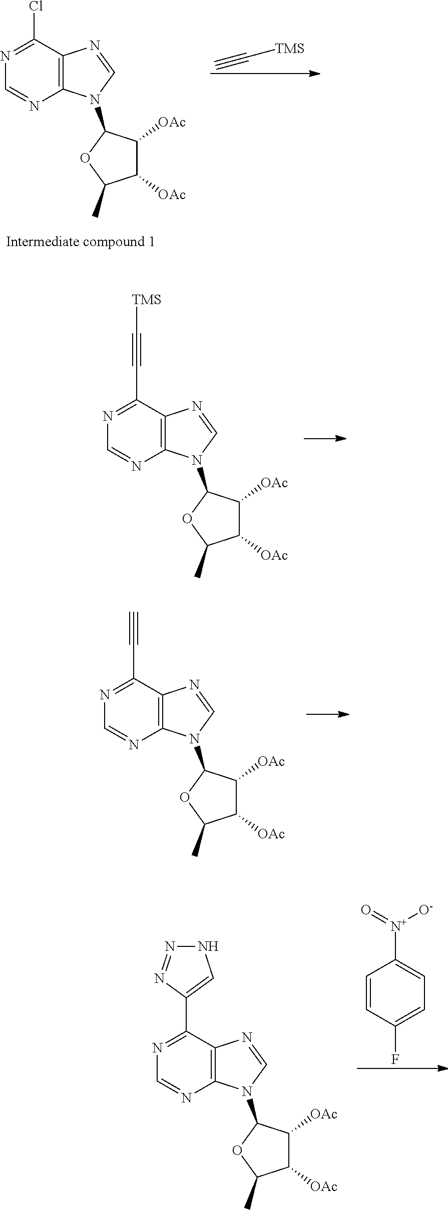

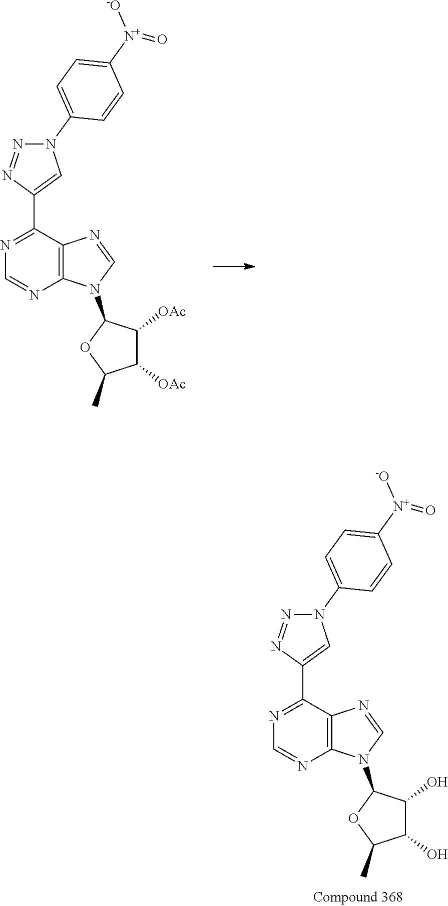

A mixture of Intermediate Compound 1 (2 g, 5.64 mmol, 1 eq), ethynyltrimethylsilane (830.61 mg, 8.46 mmol, 1.17 mL, 1.5 eq), Pd(PPh3)2Cl2 (395.72 mg, 563.79 μmol, 0.1 eq), and copper(I) iodide (214.75 mg, 1.13 mmol, 0.2 eq) in DMF (20 mL) was added TEA (1.71 g, 16.91 mmol, 2.35 mL, 3 eq). The mixture was degassed and purged with N2 3 times, and then stirred at 60° C. for 3 hr under N2 atmosphere. The reaction mixture was concentrated under reduced pressure to remove the solvent and the residue was purified by column chromatography (SiO2, Petroleum ether/Ethyl acetate=100/1 to 1/1) to afford the corresponding protected acetylene compound (1.6 g, 67% yield) as a yellow gum.

LCMS: Rt=0.848 min; (ESI positive ion) m/z: 417.2 (M+H)+(calculated: 417.15).

To a solution of the above protected acetylene compound (800 mg, 1.92 mmol, 1 eq) in DMF (10 mL) was added potassium fluoride (1.12 g, 19.21 mmol, 449.96 μL, 10 eq). The mixture was stirred at 50° C. for 1 hr. The reaction mixture was then concentrated under reduced pressure to give a residue, which was purified by column chromatography (SiO2, Petroleum ether/Ethyl acetate=100/0 to 0/1) to afford corresponding acetylene compound (600 mg, 37% yield) as a red solid.

LCMS: Rt=0.767 min; (ESI positive ion) m/z: 345.1 (M+H)+(calculated: 345.11).

To a solution of the above acetylene compound (600 mg, 1.74 mmol, 1 eq) and trimethylsilyl azide (200.76 mg, 1.74 mmol, 229.18 μL, 1 eq) in DCM (5 mL) and water (5 mL) was added copper sulfate pentahydrate (43.51 mg, 174.26 μmol, 0.1 eq) and sodiμm; (2R)-2-[(1S)-1,2-dihydroxyethyl]-4-hydroxy-5-oxo-2H-furan-3-olate (69.04 mg, 348.51 μmol, 0.2 eq). The mixture was stirred at 20° C. for 16 hr. The reaction mixture was then poured into H2O (15 mL) and the pH adjusted with a saturated aqueous solution of Na2CO3 to greater than 9 at 0° C. Then the mixture was extracted with ethyl acetate (15 mL×3). The organic layer was washed with brine (30 mL), dried by Na2SO4, filtered and concentrated under reduced pressure to give a residue. The residue was purified by Prep-TLC (SiO2, Petroleum ether/Ethyl acetate=0:1, Rf=0.2) to afford the corresponding triazole compound (180 mg, 19% yield) as a brown solid.

LCMS: Rt=0.684 min; (ESI positive ion) m/z: 388.1 (M+H)+(calculated: 388.13).

To a solution of the above triazole compound (50 mg, 129.08 μmol, 1 eq) and 1-fluoro-4-nitrobenzene (21.86 mg, 154.90 μmol, 16.43 μL, 1.2 eq) in DMF (1 mL) was added Cs2CO3 (105.14 mg, 322.71 μmol, 2.5 eq). The mixture was stirred at 80° C. for 3 hr. The reaction mixture was then concentrated under reduced pressure to give a residue, which was purified by Prep-TLC (SiO2, Petroleum ether/Ethyl acetate=1/1, Rf=0.3) to afford the corresponding nitrobenzyl compound (10 mg, 9% yield) as a white solid.

LCMS: Rt=0.945 min; (ESI positive ion) m/z: 509.2 (M+H)+(calculated: 509.15).

To a solution of the above nitrobenzyl compound (10 mg, 19.67 μmol, 1 eq) in MeOH (0.5 mL) was added NH3·H2O (1.63 g, 12.98 mmol, 1.79 mL, 28% purity, 660.11 eq). The mixture was stirred at 20° C. for 1 hr. The reaction mixture was then concentrated under reduced pressure to give a residue, which was purified by Prep-HPLC (column: Phenomenex Luna C18 150×25 mm×10 μm; mobile phase: [water(FA)-ACN]; B %: 24%-54%, 10 min) The purified solution was lyophilized to afford Compound 368 (2.1 mg, 25% yield) as a white solid.

LCMS: Rt=0.847 min; (ESI positive ion) m/z: 424.9 (M+H)+(calculated: 425.12).

1H NMR (400 MHz, MeOD-d4) δ=9.04 (d, J=12.8 Hz, 2H), 8.76 (s, 1H), 8.60-8.47 (m, 5H), 6.15 (d, J=4.5 Hz, 1H), 4.57 (br s, 1H), 4.26-4.15 (m, 2H), 1.47 (d, J=6.0 Hz, 3H).

### Compound 369:

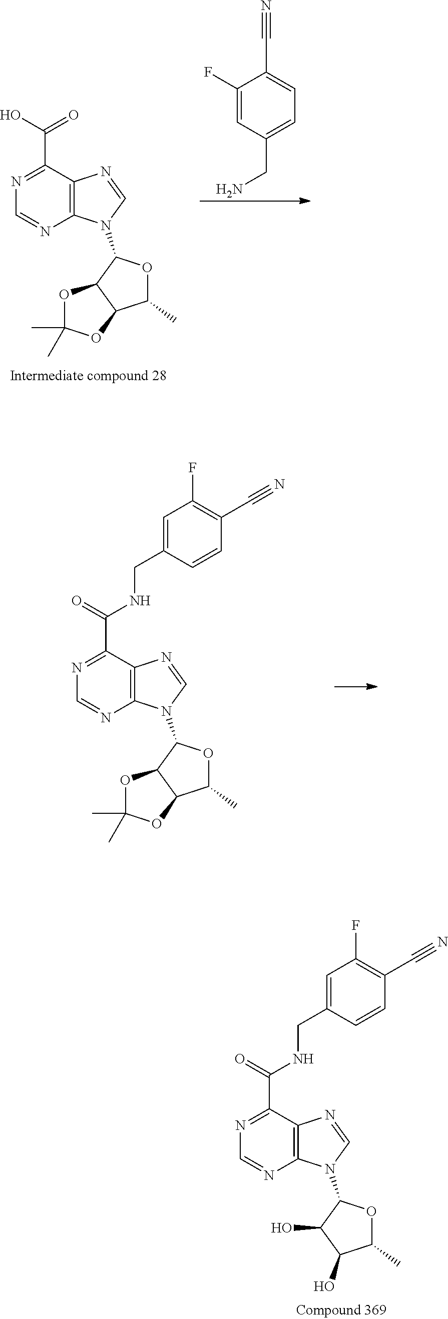 [SMILES: C[C@H]1O[C@@H](n2cnc3c(C(=O)O)ncnc32)[C@@H]2OC(C)(C)O[C@@H]21 ; N#Cc1ccc(CN)cc1F ; C[C@H]1O[C@@H](n2cnc3c(C(=O)NCc4ccc(C#N)c(F)c4)ncnc32)[C@@H]2OC(C)(C)O[C@@H]21 ; C[C@H]1O[C@@H](n2cnc3c(C(=O)NCc4ccc(C#N)c(F)c4)ncnc32)[C@H](O)[C@@H]1O]

To a solution of acetylene 28 (100 mg, 312.21 μmol, 1 eq) and 4-(aminomethyl)-2-fluorobenzonitrile (69.91 mg, 374.65 μmol, 1.2 eq, HCl) in THF (5 mL) was added DIEA (201.75 mg, 1.56 mmol, 271.90 μL, 5 eq) and T3P (496.69 mg, 780.52 μmol, 464.20 μL, 50% purity, 2.5 eq) at 0° C. Then the mixture was stirred at 20° C. for 2 hr. The reaction mixture filtered and the filtrate was concentrated under reduced pressure to give a residue. The residue was purified by Prep-TLC (SiO2, PE:EA=0:1) to afford the corresponding amide compound (132 mg, 93% yield) as a yellow oil.

LCMS: Rt=0.512 min; (ESI positive ion) m/z: 453.2 (M+H)+(calculated: 453.16).

To a solution of the above amide compound (120 mg, 265.23 μmol, 1 eq) was added formic acid/water (4/1 V/V) (5 mL) at 0° C. The reaction was stirred at 0° C. for 6 hr. NH3·H2O was then added to the reaction mixture at 0° C. until a pH was 7 was achieved. The mixture was then filtered and concentrated under reduced pressure to give a residue, which was purified by Prep-HPLC (column: Phenomenex C18 150×25 mm×10 μm; mobile phase: [water (NH4HCO3) -ACN]; B %: 6%-36%, 8 min) to give a solution. The solution was lyophilized to afford Compound 369 (44.05 mg, 38% yield) as a white solid.

LCMS: Rt=0.301 min; (ESI positive ion) m/z: 413.1 (M+H)+(calculated: 413.13).

1H NMR (400 MHz, DMSO-d6), δ=9.64-9.60 (m, 1H), 9.08 (s, 1H), 8.96 (s, 1H), 7.93-7.89 (m, 1H), 7.53 (d, J=10.6 Hz, 1H), 7.43 (dd, J=1.0, 8.0 Hz, 1H), 6.04 (d, J=5.0 Hz, 1H), 5.53 (d, J=5.6 Hz, 1H), 5.24 (d, J=5.2 Hz, 1H), 4.73 (d, J=5.2 Hz, 1H), 4.67 (d, J=6.2 Hz, 2H), 4.06-4.01 (m, 2H), 1.34 (d, J=6.0 Hz, 3H).

### Compound 371:

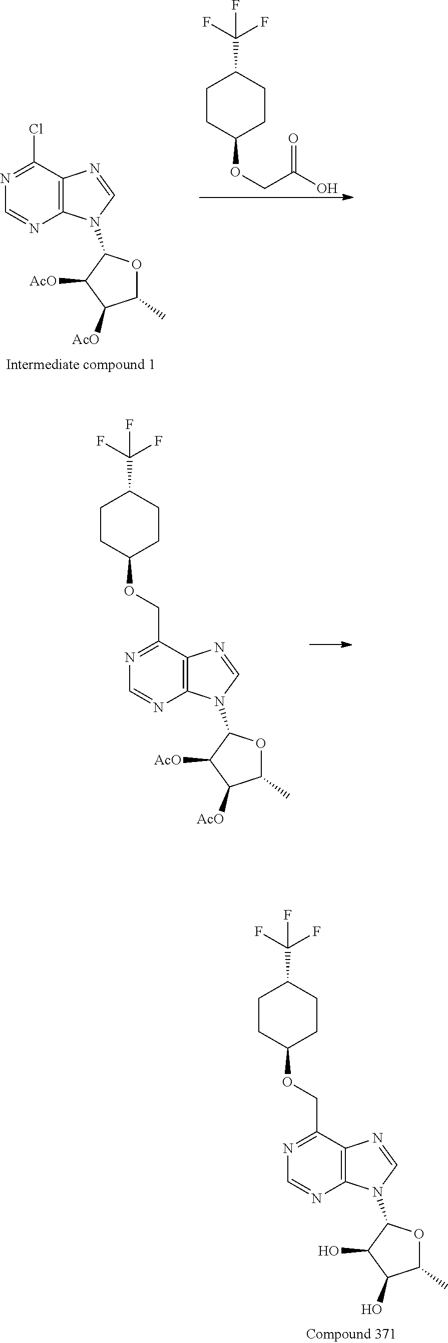 [SMILES: CC(=O)O[C@@H]1[C@H](OC(C)=O)[C@@H](C)O[C@H]1n1cnc2c(Cl)ncnc21 ; O=C(O)CO[C@H]1CC[C@H](C(F)(F)F)CC1 ; CC(=O)O[C@@H]1[C@H](OC(C)=O)[C@@H](C)O[C@H]1n1cnc2c(CO[C@H]3CC[C@H](C(F)(F)F)CC3)ncnc21 ; C[C@H]1O[C@@H](n2cnc3c(CO[C@H]4CC[C@H](C(F)(F)F)CC4)ncnc32)[C@H](O)[C@@H]1O]

Compound 371 (32.26 mg, 62% yield) was synthesized from acetylene 1 and 2-(((1R,4R)-4-(trifluoromethyl)cyclohexyl)oxy)acetic acid following the protocols described for Compound 285.

LCMS: Rt=0.719 min; (ESI positive ion) m/z: 417.2 (M+H)+(calculated: 417.17).

1H NMR (400 MHz, DMSO-d6) δ=8.92 (s, 1H), 8.78 (s, 1H), 5.99 (d, J=5.0 Hz, 1H), 5.50 (d, J=5.6 Hz, 1H), 5.22 (d, J=4.6 Hz, 1H), 4.93 (s, 2H), 4.73 (q, J=5.0 Hz, 1H), 4.02 (br d, J=3.8 Hz, 2H), 3.55-3.45 (m, 1H), 2.29-2.12 (m, 3H), 1.91-1.84 (m, 2H), 1.33 (br d, J=5.8 Hz, 3H), 1.30-1.23 (m, 4H).

### Compound 373:

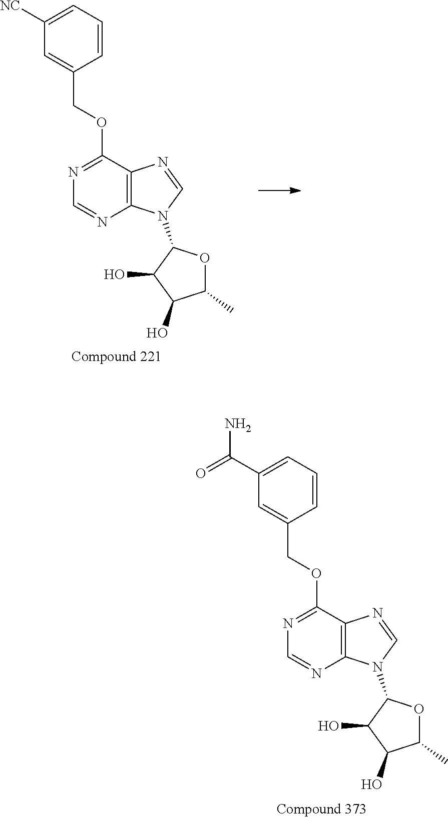 [SMILES: C[C@H]1O[C@@H](n2cnc3c(OCc4cccc(C#N)c4)ncnc32)[C@H](O)[C@@H]1O ; C[C@H]1O[C@@H](n2cnc3c(OCc4cccc(C(N)=O)c4)ncnc32)[C@H](O)[C@@H]1O]

To a solution of Compound 221 (40.00 mg, 108.89 μmol, 1 eq) in THF (1.2 mL) and water (0.4 mL) was added LiOH (13.04 mg, 544.43 μmol, 5 eq) and hydrogen peroxide (49.38 mg, 435.54 μmol, 41.85 μL, 30% purity, 4 eq). The mixture was stirred at 20° C. for 16 hr. A solution of 1N HCl was added to the reaction mixture to adjust the pH to less than 8. To the mixture was added water (30 mL) and then the mixture was extracted with EtOAc (20 mL×3). The organic layer was washed with brine and concentrated to give a residue, which was purified by Prep-HPLC (column: Waters Xbridge 150×25 mm×5 μm; mobile phase: [water(10 mM NH4HCO3)-ACN]; B %: 10%-40%, 8 min). The purified solution was lyophilized to afford Compound 373 (27.65 mg, 65% yield) as a white solid.

LCMS: Rt=0.725 min; (ESI positive ion) m/z: 386.1 (M+H)+(calculated: 386.14).

1H NMR (400 MHz, DMSO-d6) δ=8.60 (s, 1H), 8.58 (s, 1H), 8.01 (br s, 2H), 7.86 (d, J=7.7 Hz, 1H), 7.66 (d, J=7.6 Hz, 1H), 7.52-7.46 (m, 1H), 7.40 (br s, 1H), 5.94 (d, J=4.9 Hz, 1H), 5.68 (s, 2H), 5.62-5.00 (m, 2H), 4.69 (t, J=4.5 Hz, 1H), 4.00 (br d, J=4.6 Hz, 2H), 1.32 (d, J=5.9 Hz, 3H).

### Compound 379:

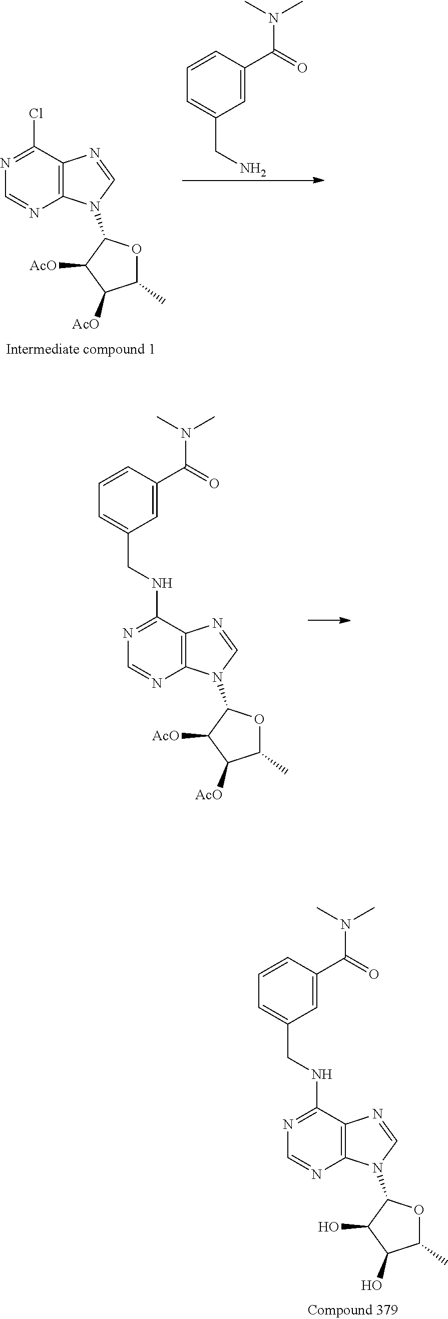 [SMILES: CC(=O)O[C@@H]1[C@H](OC(C)=O)[C@@H](C)O[C@H]1n1cnc2c(Cl)ncnc21 ; CN(C)C(=O)c1cccc(CN)c1 ; CC(=O)O[C@@H]1[C@H](OC(C)=O)[C@@H](C)O[C@H]1n1cnc2c(NCc3cccc(C(=O)N(C)C)c3)ncnc21 ; C[C@H]1O[C@@H](n2cnc3c(NCc4cccc(C(=O)N(C)C)c4)ncnc32)[C@H](O)[C@@H]1O]

Compound 379 (49.7 mg, 80% yield) was synthesized from Intermediate Compound 1 and 3-(aminomethyl)-N,N-dimethylbenzamide following the protocols described for Compound 254.

LCMS: Rt=0.736 min; (ESI positive ion) m/z: 413.1 (M+H)+(calculated: 413.19).

1H NMR (400 MHz, DMSO-d6) δ=8.44 (br d, J=6.8 Hz, 1H), 8.35 (s, 1H), 8.22 (s, 1H), 7.44-7.38 (m, 1H), 7.37-7.31 (m, 2H), 7.23 (br d, J=7.5 Hz, 1H), 5.85 (d, J=4.9 Hz, 1H), 5.43 (d, J=5.6 Hz, 1H), 5.15 (br d, J=5.0 Hz, 1H), 4.84-4.62 (m, 3H), 4.03-3.91 (m, 2H), 2.94 (br s, 3H), 2.89-2.81 (m, 3H), 1.30 (br d, J=5.9 Hz, 3H).

### Compound 380:

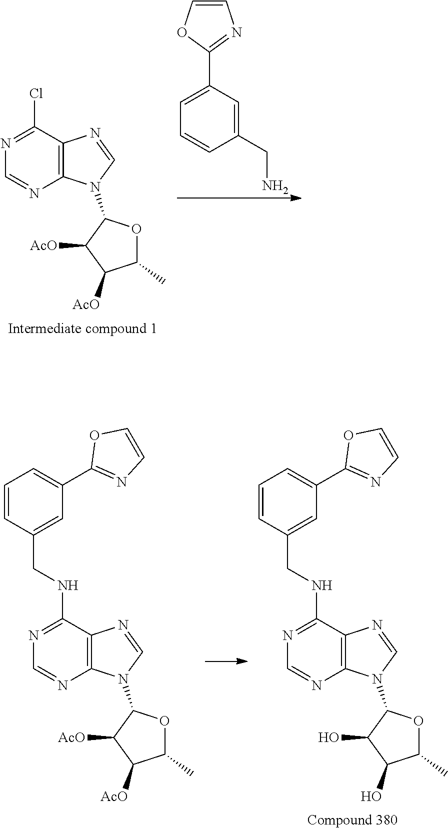 [SMILES: CC(=O)O[C@@H]1[C@H](OC(C)=O)[C@@H](C)O[C@H]1n1cnc2c(Cl)ncnc21 ; NCc1cccc(-c2ncco2)c1 ; CC(=O)O[C@@H]1[C@H](OC(C)=O)[C@@H](C)O[C@H]1n1cnc2c(NCc3cccc(-c4ncco4)c3)ncnc21 ; C[C@H]1O[C@@H](n2cnc3c(NCc4cccc(-c5ncco5)c4)ncnc32)[C@H](O)[C@@H]1O]

Compound 380 (13.6 mg, 36% yield) was synthesized from Intermediate Compound 1 and (3-(oxazol-2-yl)phenyl)methanamine following the protocols described for Compound 254.

LCMS: Rt=0.767 min; (ESI positive ion) m/z: 409.3 (M+H)+(calculated: 409.15).

1H NMR (400 MHz, DMSO-d6) δ=8.59-8.48 (m, 1H), 8.37 (s, 1H), 8.21 (br d, J=15.2 Hz, 2H), 7.98 (s, 1H), 7.83 (br d, J=7.2 Hz, 1H), 7.47 (br d, J=9.2 Hz, 2H), 7.34 (s, 1H), 5.85 (br s, 1H), 5.45 (br s, 1H), 5.16 (br s, 1H), 4.78 (br s, 1H), 4.68 (br s, 1H), 3.97 (br d, J=3.6 Hz, 2H), 1.30 (br d, J=5.6 Hz, 3H).

### Compound 387:

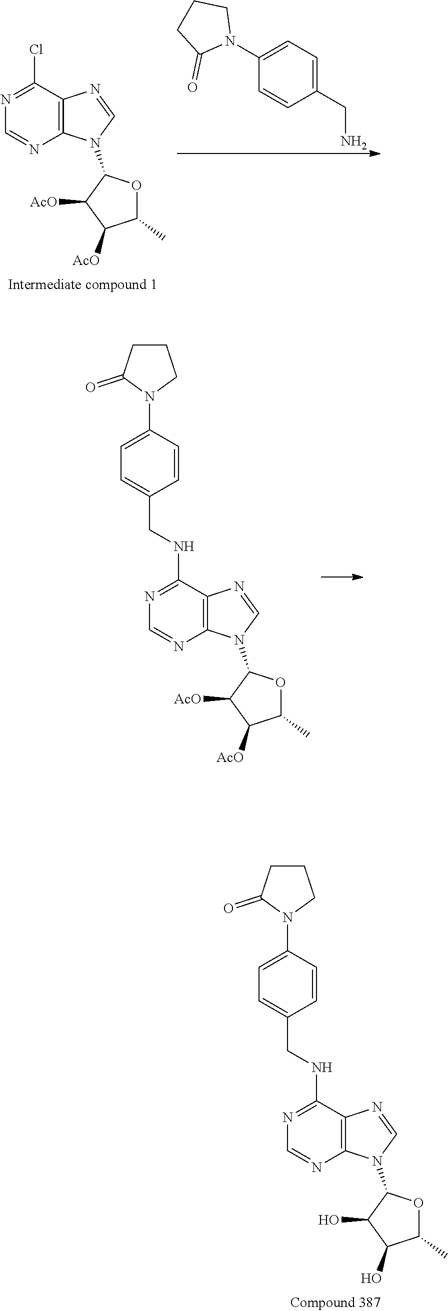 [SMILES: CC(=O)O[C@@H]1[C@H](OC(C)=O)[C@@H](C)O[C@H]1n1cnc2c(Cl)ncnc21 ; NCc1ccc(N2CCCC2=O)cc1 ; CC(=O)O[C@@H]1[C@H](OC(C)=O)[C@@H](C)O[C@H]1n1cnc2c(NCc3ccc(N4CCCC4=O)cc3)ncnc21 ; C[C@H]1O[C@@H](n2cnc3c(NCc4ccc(N5CCCC5=O)cc4)ncnc32)[C@H](O)[C@@H]1O]

Compound 387 (22.55 mg, 54% yield) was synthesized from Intermediate Compound 1 and 1-(4-(aminomethyl)phenyl)pyrrolidin-2-one following the protocols described for Compound 254.

LCMS: Rt=0.728 min; (ESI positive ion) m/z: 425.3 (M+H)+(calculated: 425.19).

1H NMR (400 MHz, DMSO-d6) δ=8.34 (s, 2H), 8.21 (br s, 1H), 7.55 (br d, J=7.6 Hz, 2H), 7.33 (br d, J=7.8 Hz, 2H), 5.85 (br d, J=4.6 Hz, 1H), 5.42 (br d, J=4.8 Hz, 1H), 5.14 (br d, J=2.8 Hz, 1H), 4.66 (br d, J=0.8 Hz, 3H), 3.97 (br d, J=4.8 Hz, 2H), 3.78 (br t, J=6.8 Hz, 2H), 2.46-2.43 (m, 2H), 2.10-1.98 (m, 2H), 1.30 (br d, J=5.6 Hz, 3H).

1H NMR (400 MHz, DMSO-d6+D2O) δ=8.29 (s, 1H), 8.19 (s, 1H), 7.51 (d, J=8.8 Hz, 2H), 7.32 (d, J=8.6 Hz, 2H), 5.83 (d, J=5.0 Hz, 1H), 4.65 (br d, J=5.0 Hz, 3H), 4.03-3.93 (m, 2H), 3.77 (t, J=7.2 Hz, 2H), 2.45 (t, J=8.2 Hz, 2H), 2.02 (t, J=7.6 Hz, 2H), 1.28 (d, J=6.0 Hz, 3H).

### Compound 399:

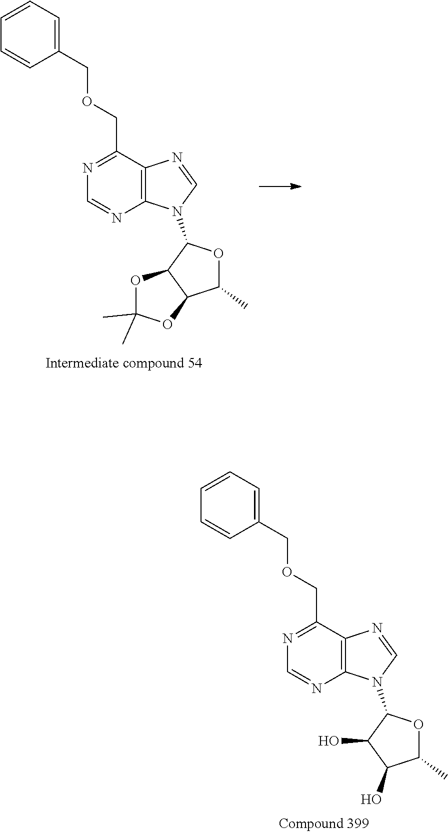 [SMILES: C[C@H]1O[C@@H](n2cnc3c(COCc4ccccc4)ncnc32)[C@@H]2OC(C)(C)O[C@@H]21 ; C[C@H]1O[C@@H](n2cnc3c(COCc4ccccc4)ncnc32)[C@H](O)[C@@H]1O]

A solution of Intermediate Compound 54 (30 mg, 75.67 μmol, 1 eq) in water (0.1 mL) and formic acid (0.4 mL) was stirred at 0° C. for 1 hr. The pH of the mixture was adjusted to about 8 by NaHCO3. The mixture was then filtered and concentrated under vacuum. The residue was purified by Prep-HPLC (column: Waters Xbridge 150×25 mm×5 μm; mobile phase: [water(10 mM NH4HCO3)-ACN]; B %: 16%-46%, 8 min) to afford Compound 399 (6.02 mg, 22% yield) as a colorless gum.

LCMS: Rt=0.767 min; (ESI positive ion) m/z: 356.9 (M+H)+(calculated: 357.15).

1H NMR (400 MHz, DMSO-d6) δ=8.94 (s, 1H), 8.80 (s, 1H), 7.41-7.26 (m, 5H), 6.00 (d, J=5.0 Hz, 1H), 5.51 (br d, J=5.4 Hz, 1H), 5.23 (br d, J=4.4 Hz, 1H), 4.97 (s, 2H), 4.74 (br d, J=4.8 Hz, 1H), 4.67 (s, 2H), 4.06-3.99 (m, 2H), 1.33 (d, J=6.0 Hz, 3H).

### Compound 400:

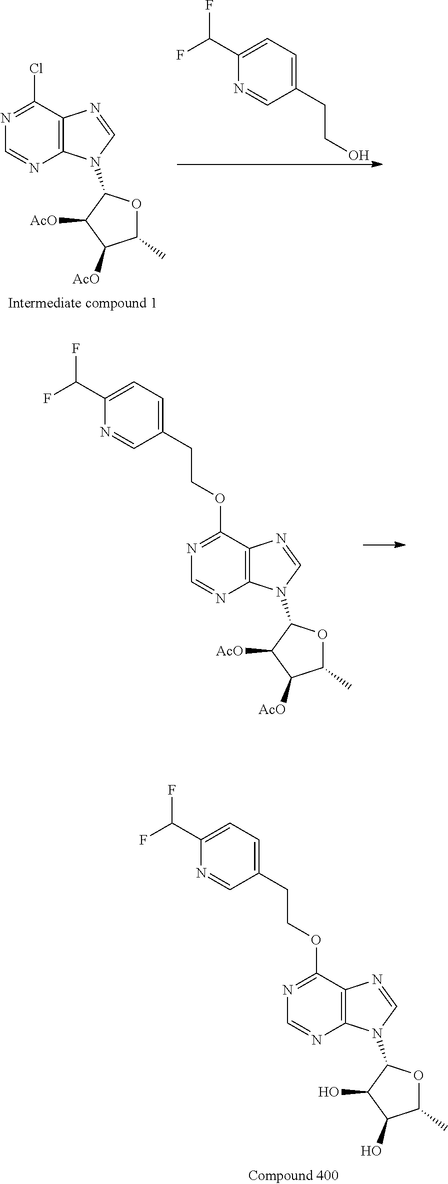 [SMILES: CC(=O)O[C@@H]1[C@H](OC(C)=O)[C@@H](C)O[C@H]1n1cnc2c(Cl)ncnc21 ; OCCc1ccc(C(F)F)nc1 ; CC(=O)O[C@@H]1[C@H](OC(C)=O)[C@@H](C)O[C@H]1n1cnc2c(OCCc3ccc(C(F)F)nc3)ncnc21 ; C[C@H]1O[C@@H](n2cnc3c(OCCc4ccc(C(F)F)nc4)ncnc32)[C@H](O)[C@@H]1O]

Compound 400 (48.5 mg, 34% yield) was synthesized from Intermediate Compound 1 and 2-(6-(difluoromethyl)pyridin-3-yl)ethan-1-ol following the protocols described for Compound 188.

LCMS: Rt=0.776 min; (ESI positive ion) m/z: 407.9 (M+H)+(calculated: 408.14).

1H NMR (400 MHz, DMSO-d6) δ=8.66 (s, 1H), 8.55 (d, J=10.6 Hz, 2H), 7.98 (dd, J=1.8, 7.8 Hz, 1H), 7.65 (d, J=7.8 Hz, 1H), 7.09-6.76 (m, 1H), 5.93 (d, J=4.8 Hz, 1H), 5.50 (br s, 1H), 5.23 (br s, 1H), 4.83 (t, J=6.6 Hz, 2H), 4.67 (br s, 1H), 4.04-3.95 (m, 2H), 3.25 (br t, J=6.4 Hz, 2H), 1.31 (d, J=6.0 Hz, 3H).

### Compound 401:

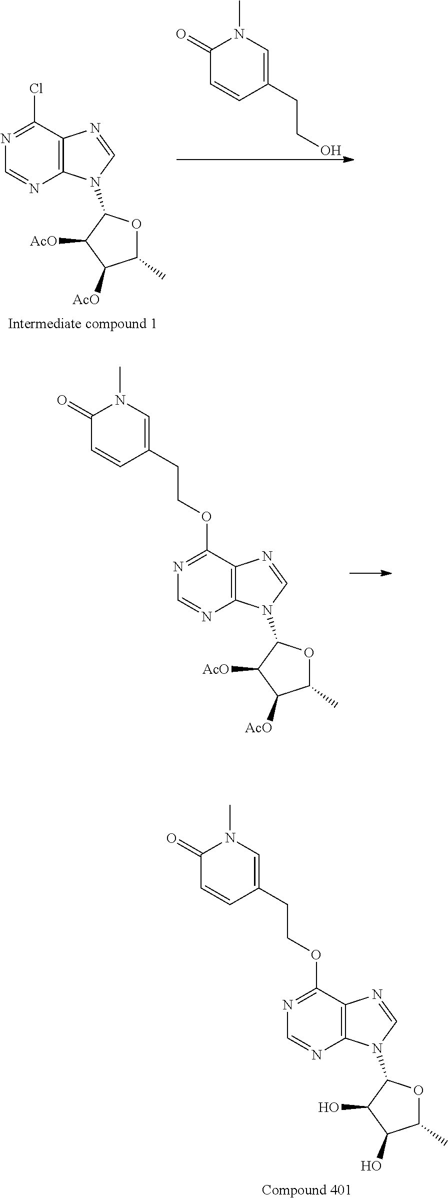 [SMILES: CC(=O)O[C@@H]1[C@H](OC(C)=O)[C@@H](C)O[C@H]1n1cnc2c(Cl)ncnc21 ; Cn1cc(CCO)ccc1=O ; CC(=O)O[C@@H]1[C@H](OC(C)=O)[C@@H](C)O[C@H]1n1cnc2c(OCCc3ccc(=O)n(C)c3)ncnc21 ; C[C@H]1O[C@@H](n2cnc3c(OCCc4ccc(=O)n(C)c4)ncnc32)[C@H](O)[C@@H]1O]

Compound 401 (35 mg, 36% yield) was synthesized from Intermediate Compound 1 and 5-(2-hydroxyethyl)-1-methylpyridin-2(1H)-one following the protocols described for Compound 188.

LCMS: Rt=0.703 min; (ESI positive ion) m/z: 387.9 (M+H)+(calculated: 388.15).

1H NMR (400 MHz, DMSO-d6) δ=8.58 (s, 1H), 8.54 (s, 1H), 7.63 (d, J=2.4 Hz, 1H), 7.46 (dd, J=2.6, 9.4 Hz, 1H), 6.35 (d, J=9.4 Hz, 1H), 5.93 (d, J=5.0 Hz, 1H), 5.48 (br d, J=5.4 Hz, 1H), 5.21 (br d, J=4.4 Hz, 1H), 4.68 (t, J=6.8 Hz, 3H), 4.04-3.96 (m, 2H), 3.38 (s, 3H), 2.88 (t, J=6.8 Hz, 2H), 1.31 (d, J=6.2 Hz, 3H).

### Compound 402:

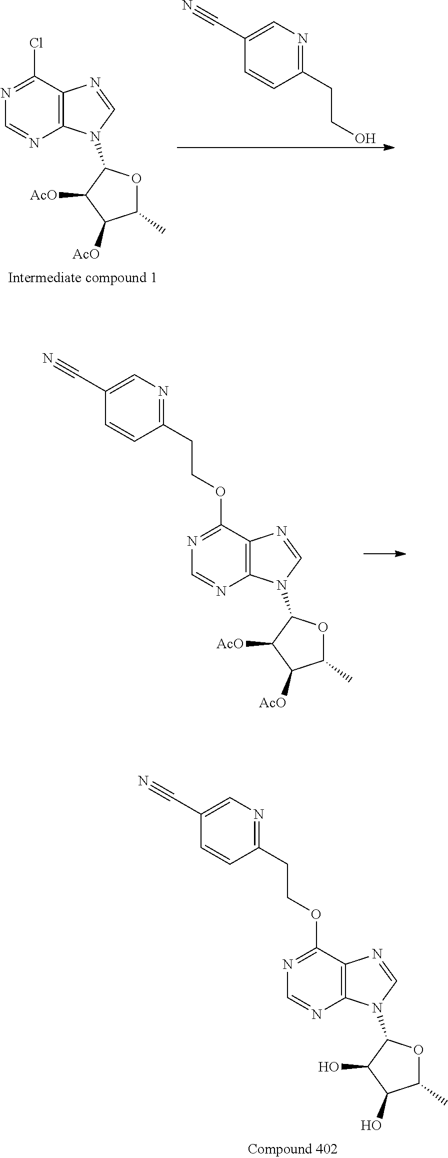 [SMILES: CC(=O)O[C@@H]1[C@H](OC(C)=O)[C@@H](C)O[C@H]1n1cnc2c(Cl)ncnc21 ; N#Cc1ccc(CCO)nc1 ; CC(=O)O[C@@H]1[C@H](OC(C)=O)[C@@H](C)O[C@H]1n1cnc2c(OCCc3ccc(C#N)cn3)ncnc21 ; C[C@H]1O[C@@H](n2cnc3c(OCCc4ccc(C#N)cn4)ncnc32)[C@H](O)[C@@H]1O]

Compound 402 (1.7 mg, 5% yield) was synthesized from Intermediate Compound 1 and 6-(2-hydroxyethyl)nicotinonitrile following the protocols described for Compound 188.

LCMS: Rt=0.732 min; (ESI positive ion) m/z: 382.9 (M+H)+(calculated: 383.14).

1H NMR (400 MHz, MeOD-d4) δ=8.84 (d, J=2.0 Hz, 1H), 8.56-8.52 (m, 1H), 8.51 (s, 1H), 8.38 (s, 1H), 8.08 (dd, J=2.1, 8.2 Hz, 1H), 7.62 (d, J=7.9 Hz, 1H), 6.01 (d, J=4.5 Hz, 1H), 5.04 (t, J=6.3 Hz, 2H), 4.76 (br d, J=4.5 Hz, 1H), 4.16-4.09 (m, 2H), 3.46 (t, J=6.4 Hz, 2H), 1.42 (d, J=6.0 Hz, 3H).

### Compound 403:

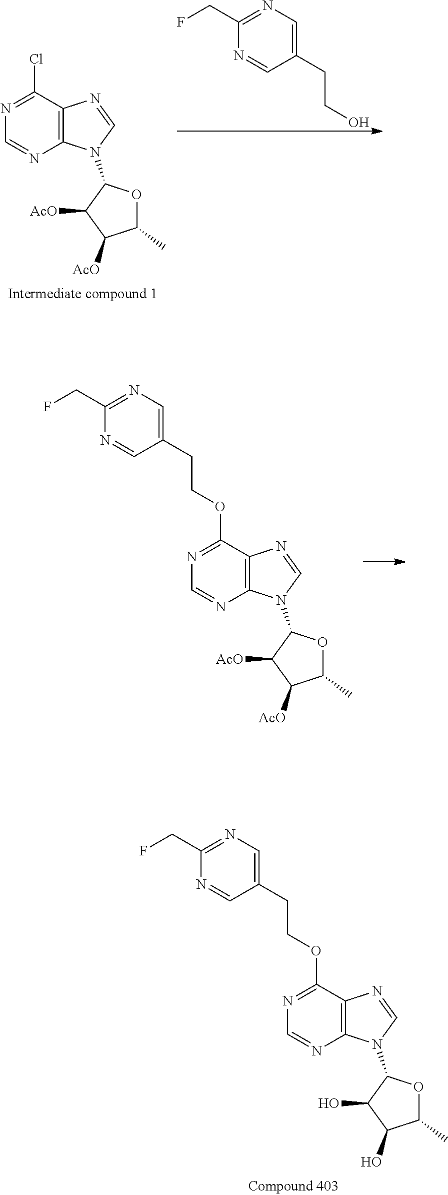 [SMILES: CC(=O)O[C@@H]1[C@H](OC(C)=O)[C@@H](C)O[C@H]1n1cnc2c(Cl)ncnc21 ; OCCc1cnc(CF)nc1 ; CC(=O)O[C@@H]1[C@H](OC(C)=O)[C@@H](C)O[C@H]1n1cnc2c(OCCc3cnc(CF)nc3)ncnc21 ; C[C@H]1O[C@@H](n2cnc3c(OCCc4cnc(CF)nc4)ncnc32)[C@H](O)[C@@H]1O]

Compound 403 (2.65 mg, 8% yield) was synthesized from Intermediate Compound 1 and 2-(2-(fluoromethyl)pyrimidin-5-yl)ethan-1-ol following the protocols described for Compound 188.

LCMS: Rt=0.681 min; (ESI positive ion) m/z: 391.0 (M+H)+(calculated: 391.15).

1H NMR (400 MHz, DMSO-d6) δ=8.85 (s, 2H), 8.58 (s, 1H), 8.54 (s, 1H), 5.93 (d, J=4.8 Hz, 1H), 5.54 (s, 1H), 5.48 (d, J=5.7 Hz, 1H), 5.43 (s, 1H), 5.21 (d, J=4.9 Hz, 1H), 4.83 (br t, J=6.3 Hz, 2H), 4.67 (br d, J=4.9 Hz, 1H), 3.99 (br d, J=4.3 Hz, 2H), 3.21 (br t, J=6.2 Hz, 2H), 1.31 (br d, J=5.9 Hz, 3H).

### Compound 404:

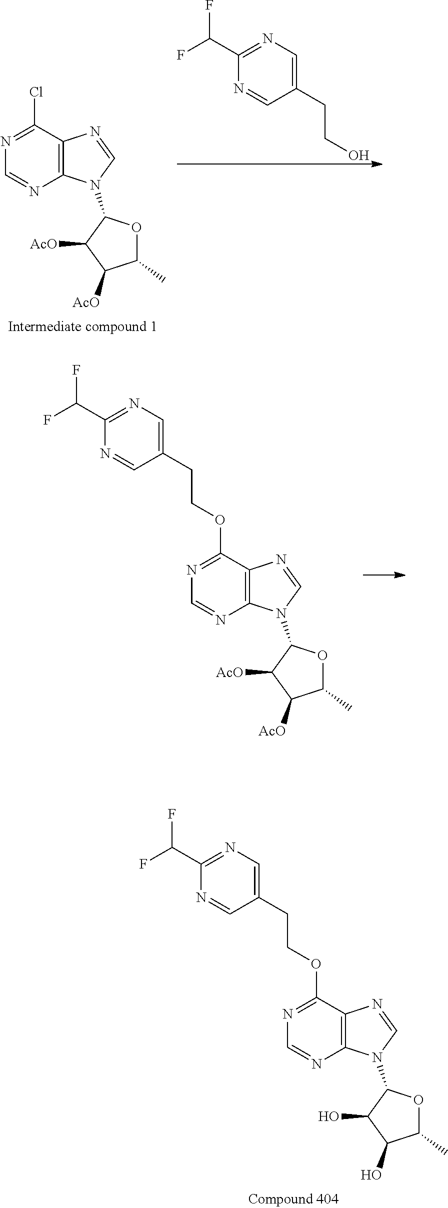 [SMILES: CC(=O)O[C@@H]1[C@H](OC(C)=O)[C@@H](C)O[C@H]1n1cnc2c(Cl)ncnc21 ; OCCc1cnc(C(F)F)nc1 ; CC(=O)O[C@@H]1[C@H](OC(C)=O)[C@@H](C)O[C@H]1n1cnc2c(OCCc3cnc(C(F)F)nc3)ncnc21 ; C[C@H]1O[C@@H](n2cnc3c(OCCc4cnc(C(F)F)nc4)ncnc32)[C@H](O)[C@@H]1O]

Compound 404 (28.4 mg, 25% yield) was synthesized from Intermediate Compound 1 and 2-(2-(difluoromethyl)pyrimidin-5-yl)ethan-1-ol following the protocols described for Compound 188.

LCMS: Rt=0.721 min; (ESI positive ion) m/z: 408.9 (M+H)+(calculated: 409.14).

1H NMR (400 MHz, DMSO-d6) δ=8.98 (s, 2H), 8.58 (s, 1H), 8.54 (s, 1H), 7.09-6.80 (m, 1H), 5.93 (d, J=5.0 Hz, 1H), 5.49 (br d, J=5.4 Hz, 1H), 5.22 (br d, J=3.8 Hz, 1H), 4.85 (t, J=6.2 Hz, 2H), 4.70-4.64 (m, 1H), 4.02-3.96 (m, 2H), 3.27 (br t, J=6.2 Hz, 2H), 1.31 (d, J=6.0 Hz, 3H).

### Compound 405:

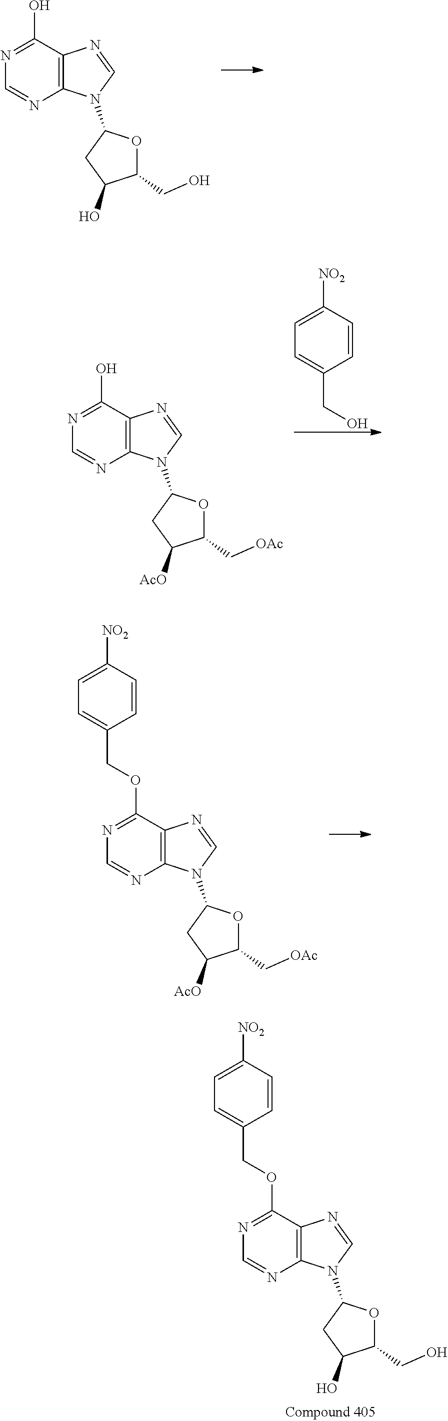 [SMILES: OC[C@H]1O[C@@H](n2cnc3c(O)ncnc32)C[C@@H]1O ; CC(=O)OC[C@H]1O[C@@H](n2cnc3c(O)ncnc32)C[C@@H]1OC(C)=O ; O=[N+]([O-])c1ccc(CO)cc1 ; CC(=O)OC[C@H]1O[C@@H](n2cnc3c(OCc4ccc([N+](=O)[O-])cc4)ncnc32)C[C@@H]1OC(C)=O ; O=[N+]([O-])c1ccc(COc2ncnc3c2ncn3[C@H]2C[C@H](O)[C@@H](CO)O2)cc1]

To a solution of 9-((2R,4S,5R)-4-hydroxy-5-(hydroxymethyl)tetrahydrofuran-2-yl)-9H-purin-6-ol (2 g, 7.93 mmol, 1 eq) in dry pyridine (6.5 mL) was added acetic anhydride (4.86 g, 47.58 mmol, 4.46 mL, 6 eq) at 0° C. The mixture was stirred at 20° C. for 16 hr. The solvent was removed under reduced pressure to give a crude product. The crude product was triturated with EtOAc (5 mL) at 20° C. for 60 min to afford the corresponding acetylated compound (2.5 g, 94% yield) as a white solid.

1H NMR (400 MHz, DMSO-d6) δ=12.41 (br s, 1H), 8.30 (s, 1H), 8.07 (s, 1H), 6.33 (dd, J=6.4, 7.8 Hz, 1H), 5.41-5.32 (m, 1H), 4.34-4.15 (m, 3H), 3.10-2.98 (m, 1H), 2.55 (ddd, J=2.8, 6.2, 14.4 Hz, 1H), 2.09 (s, 3H), 2.02 (s, 3H).

To a mixture of the above acetylated compound (200 mg, 594.71 μmol, 1 eq), (4-nitrophenyl)methanol (109.28 mg, 713.65 μmol, 1.2 eq) and triphenylphosphine (467.95 mg, 1.78 mmol, 3 eq) in dioxane (2 mL) was added DEAD (310.71 mg, 1.78 mmol, 324.34 μL, 3 eq) dropwise under N2 atmosphere. The mixture was stirred at 20° C. for 1 hr. The solvent was then removed under reduced pressure to give a residue, which was purified by Prep-TLC (SiO2, PE:EtOAc=1:1) to afford the corresponding ether compound (250 mg, 89% yield) as a colorless oil.

1H NMR (400 MHz, DMSO-d6) δ=8.67 (s, 1H), 8.36 (s, 1H), 8.21 (d, J=8.8 Hz, 2H), 7.56 (d, J=8.6 Hz, 2H), 6.35 (t, J=7.2 Hz, 1H), 5.42-5.33 (m, 3H), 4.33-4.16 (m, 3H), 3.05 (td, J=7.2, 14.2 Hz, 1H), 2.59 (br dd, J=2.6, 6.2 Hz, 1H), 2.09 (s, 3H), 2.01 (s, 3H). To a solution of the above ether compound (240 mg, 509.10 μmol, 1 eq) in MeOH (4 mL) was added NH3·H2O (910.00 mg, 6.49 mmol, 1 mL, 25% purity, 12.75 eq). The mixture was stirred at 20° C. for 1 hr. The solvent was then removed under reduced pressure to give a residue, which was purified by Prep-HPLC (column: Phenomenex C18 75×30 mm×3 μm; mobile phase: [water(FA)-ACN]; B %: 12%-42%, 7 min). The purified solution was lyophilized to give the crude product. The crude product was further purified by Prep-HPLC (column: Waters Xbridge 150×25 mm×5 μm; mobile phase: [water (ammonia hydroxide v/v)-ACN]; B %: 4%-34%, 9 min). The purified solution was lyophilized to afford Compound 405 (31.02 mg, 16% yield) as a white solid.

LCMS: Rt=0.761 min; (ESI positive ion) m/z: 388.1 (M+H)+(calculated: 388.12).

1H NMR (400 MHz, DMSO-d6) δ=8.64 (s, 1H), 8.35 (s, 1H), 8.20 (d, J=8.6 Hz, 2H), 7.56 (d, J=8.6 Hz, 2H), 6.32 (t, J=6.8 Hz, 1H), 5.36 (s, 3H), 4.98 (br s, 1H), 4.45-4.33 (m, 1H), 3.91-3.82 (m, 1H), 3.62-3.57 (m, 1H), 3.51 (br dd, J=4.8, 11.8 Hz, 1H), 2.64 (td, J=6.6, 13.2 Hz, 1H), 2.31 (ddd, J=3.4, 6.2, 13.2 Hz, 1H).

### Compound 406:

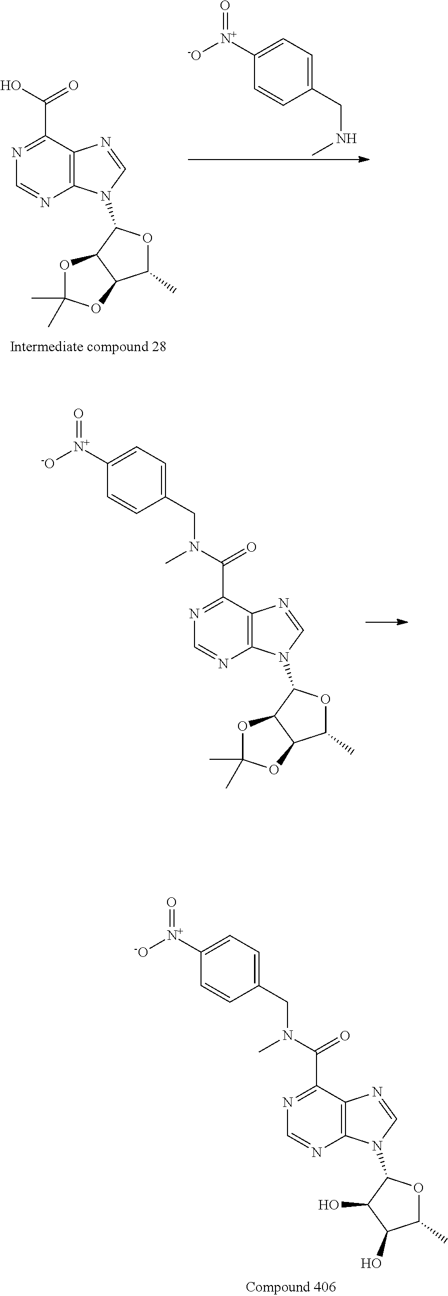

Compound 406 (6.03 mg, 13% yield) was synthesized from Intermediate Compound 28 and N-methyl-1-(4-nitrophenyl)methanamine following the protocols described for Compound 360.

LCMS: Rt=0.757 min; (ESI positive ion) m/z: 428.9 (M+H)+(calculated: 429.14).

1H NMR (400 MHz, DMSO-d6) δ=8.97 (br s, 1H), 8.70 (br s, 1H), 8.32-8.05 (m, 2H), 7.84-7.47 (m, 2H), 6.03 (br s, 1H), 5.08-4.87 (m, 2H), 4.74 (br s, 2H), 4.63-4.46 (m, 1H), 4.11-3.99 (m, 2H), 3.11-2.88 (m, 3H), 1.38 (br d, J=5.8 Hz, 3H).

### Compound 407:

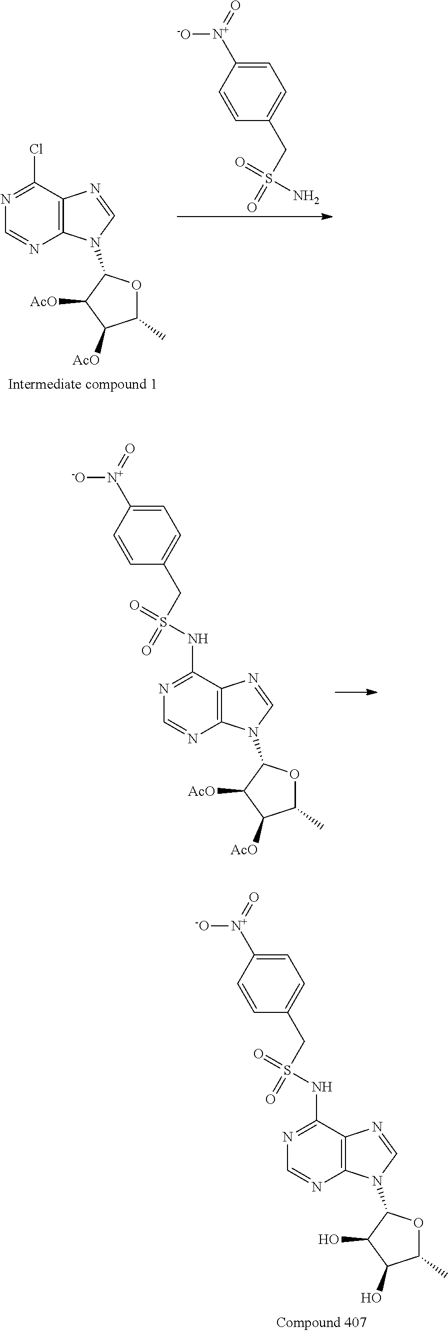

To a solution of Intermediate Compound 1 (80 mg, 225.51 μmol, 1 eq) in dioxane (3 mL) was added Pd2(dba)3 (10.33 mg, 11.28 μmol, 0.05 eq) and (4-nitrophenyl)methanesulfonamide (63.39 mg, 293.17 μmol, 1.3 eq), XPhos (16.13 mg, 33.83 μmol, 0.15 eq), and Cs2CO3 (146.95 mg, 451.03 μmol, 2 eq). The mixture was stirred at 90° C. for 5 hr. The reaction mixture was concentrated to give a residue, which was purified by column chromatography (SiO2, Petroleum ether/Ethyl acetate=0/1 to DCM:MeOH=10:1, Rf=0.6) to afford the corresponding sulfonamide compound (80 mg, 67% yield) as a red oil.

LCMS: Rt=0.893 min; (ESI positive ion) m/z: 535.0 (M+H)+(calculated: 535.12).

To a solution of the above sulfonamide compound (80 mg, 149.67 μmol, 1 eq) in MeOH (1 mL) was added NH3·H2O (910.00 mg, 7.27 mmol, 1 mL, 28% purity, 48.58 eq). The mixture was stirred at 20° C. for 1 hr. The reaction mixture was then concentrated to give a residue, which was purified by Prep-HPLC (column: Phenomenex luna C18 150×25 mm×10 μm; mobile phase: [water(FA)-ACN]; B %: 11%-41%, 10 min). The purified solution was lyophilized to afford Compound 407 (53.7 mg, 79% yield) as a white solid.

LCMS: Rt=0.730 min; (ESI positive ion) m/z: 451.0 (M+H)+(calculated: 451.10).

1H NMR (400 MHz, DMSO-d6) δ=8.54 (s, 1H), 8.38 (br s, 1H), 8.18 (d, J=8.8 Hz, 2H), 7.64 (d, J=8.7 Hz, 2H), 5.90 (d, J=4.9 Hz, 1H), 5.52 (d, J=5.6 Hz, 1H), 5.23 (br d, J=5.0 Hz, 1H), 4.94 (br s, 2H), 4.65-4.58 (m, 1H), 4.04-3.94 (m, 2H), 1.32 (d, J=6.4 Hz, 3H).

### Compound 408:

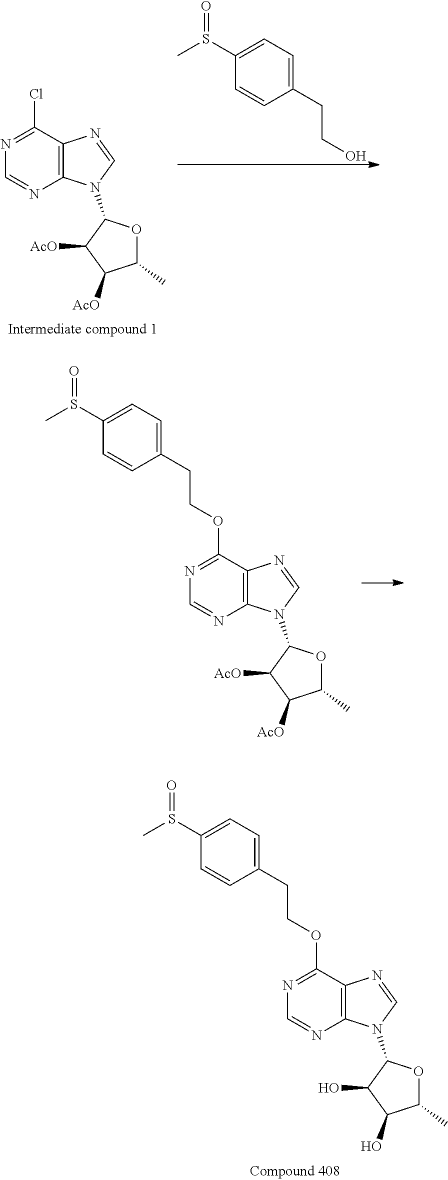

Compound 408 (7.39 mg, 900 yield) was synthesized from Intermediate Compound 1 and 2-(4-(methylsulfinyl)phenyl)ethan-1-ol following the protocols described for Compound 188.

LCMS: Rt=0.725 min; (ESI positive ion) m/z: 418.9 (M+H)+(calculated: 419.13).

1H NMR (400 MHz, DMSO-d6) δ=8.56 (d, J=10.4 Hz, 2H), 7.65-7.60 (m, 2H), 7.58-7.52 (m, 2H), 5.93 (d, J=5.2 Hz, 1H), 5.51 (br d, J=1.2 Hz, 1H), 5.24 (br s, 1H), 4.81 (t, J=6.7 Hz, 2H), 4.68 (br s, 1H), 3.99 (br d, J=4.8 Hz, 2H), 3.22 (br t, J=6.8 Hz, 2H), 2.72 (s, 3H), 1.31 (br d, J=6.1 Hz, 3H).

### Compound 409:

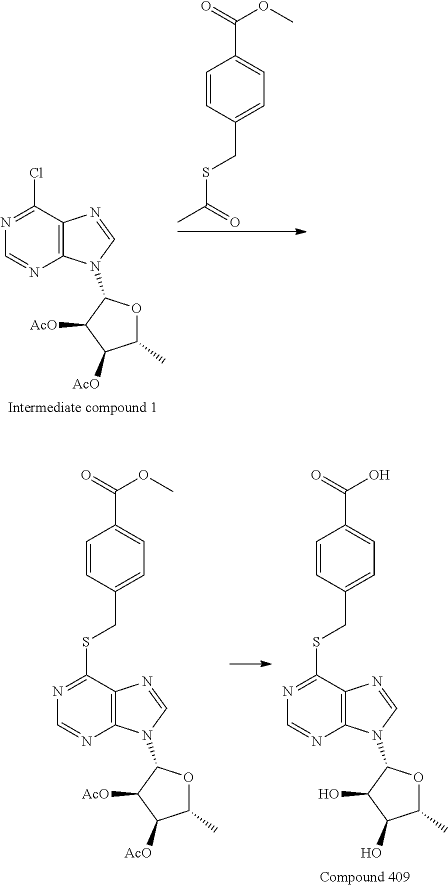 [SMILES: CC(=O)O[C@@H]1[C@H](OC(C)=O)[C@@H](C)O[C@H]1n1cnc2c(Cl)ncnc21 ; COC(=O)c1ccc(CSC(C)=O)cc1 ; COC(=O)c1ccc(CSc2ncnc3c2ncn3[C@@H]2O[C@H](C)[C@@H](OC(C)=O)[C@H]2OC(C)=O)cc1 ; C[C@H]1O[C@@H](n2cnc3c(SCc4ccc(C(=O)O)cc4)ncnc32)[C@H](O)[C@@H]1O]

To a mixture of Intermediate Compound 1 (250 mg, 704.73 μmol, 1 eq) and methyl 4-((acetylthio)methyl)benzoate (2, 205.47 mg, 916.15 μmol, 1.3 eq) in MeOH (4 mL) and THE (1 mL) was added K2CO3 (194.80 mg, 1.41 mmol, 2 eq) in one portion at 0° C. The mixture was stirred at 0° C. for 2 hours and then filtered and concentrated under reduced pressure to give a residue. The residue was purified by Prep-HPLC (column: Unisil 3-100 C18 Ultra 150×50 mm×3 μm; mobile phase: [water(FA)-ACN]; B %: 25%-55%, 15 min) to afford the corresponding thioether compound (75 mg, 25% yield) as a yellow oil.

LCMS: Rt=0.860 min; (ESI positive ion) m/z: 417.1 (M+H)+(calculated: 417.12).

To a solution of the above thioether compound (35 mg, 84.04 μmol, 1 eq) in MeOH (1 mL), THE (1 mL), and H2O (1 mL) was added lithium hydroxide monohydrate (7.05 mg, 168.09 μmol, 2 eq). The mixture was stirred at 25° C. for 6 hours. The reaction mixture was then filtered and concentrated under reduced pressure to give a residue. The residue was purified by Prep-HPLC (column: Unisil 3-100 C18 Ultra 150×50 mm×3 μm; mobile phase: [water(FA)-ACN]; B %: 12%-42%, 10 min) to afford Compound 409 (14.4 mg, 42% yield) as a white solid. LCMS: Rt=0.803 min; (ESI positive ion) m/z: 403.1 (M+H)+(calculated: 403.10).

1H NMR (400 MHz, MeOD-d4) δ=8.74 (s, 1H), 8.46 (s, 1H), 7.95 (d, J=8.1 Hz, 2H), 7.57 (d, J=8.2 Hz, 2H), 6.03 (d, J=4.5 Hz, 1H), 4.78 (t, J=4.5 Hz, 1H), 4.74 (s, 2H), 4.17-4.09 (m, 2H), 1.43 (d, J=6.0 Hz, 3H).

### Compound 410:

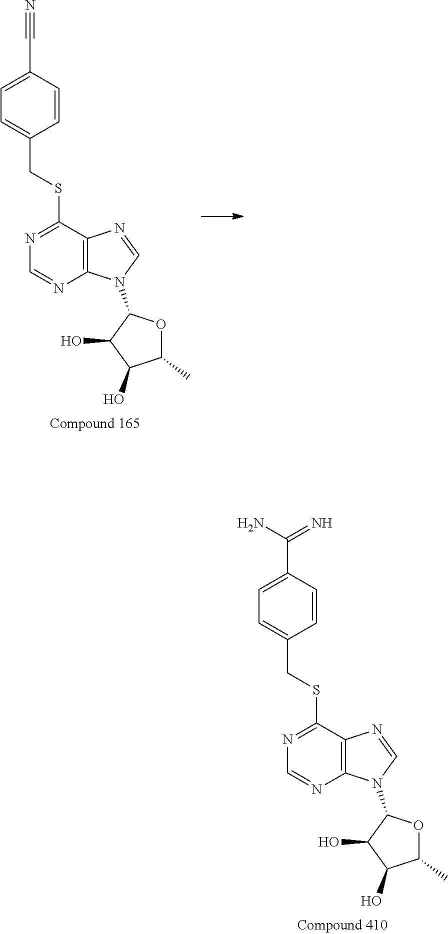 [SMILES: C[C@H]1O[C@@H](n2cnc3c(SCc4ccc(C#N)cc4)ncnc32)[C@H](O)[C@@H]1O ; C[C@H]1O[C@@H](n2cnc3c(SCc4ccc(C(=N)N)cc4)ncnc32)[C@H](O)[C@@H]1O]

To a solution of NH4Cl (209.26 mg, 3.91 mmol, 30 eq) in toluene (5 mL) was added trimethylaluminium (2 M, 2 mL, 30.67 eq) at 0° C. The reaction was stirred at 20° C. for 2 hr. Then Compound 165 (50 mg, 130.40 μmol, 1 eq) was added and the mixture was stirred at 80° C. for 16 hr. To the mixture was added water (0.5 mL) at 0-10° C. Then MeOH (5 mL) was added and the mixture was stirred at 20° C. for 0.5 hr. The mixture was then filtered and washed MeOH (5 mL×2). The filtrate was concentrated under reduced pressure to give a residue, which was purified by Prep-HPLC (column: Phenomenex C18 75×30 mm×3 μm; mobile phase: [water(FA)-ACN]; B %: 0%-30%, 7 min) to give Compound 410 (5 mg, 8% yield, formic acid salt) as a white solid.

LCMS: Rt=0.570 min; (ESI positive ion) m/z: 401.2 (M+H)+(calculated: 401.13).

1H NMR (400 MHz, DMSO-d6) δ=9.82 (s, 2H), 8.79 (s, 1H), 8.68 (s, 1H), 8.44 (s, 1H), 7.76-7.62 (m, 4H), 5.94 (d, J=5.2 Hz, 1H), 5.36 (s, 1H), 4.75 (s, 2H), 4.71-4.65 (m, 1H), 4.03-3.95 (m, 2H), 1.31 (d, J=6.0 Hz, 3H).

### Compound 411:

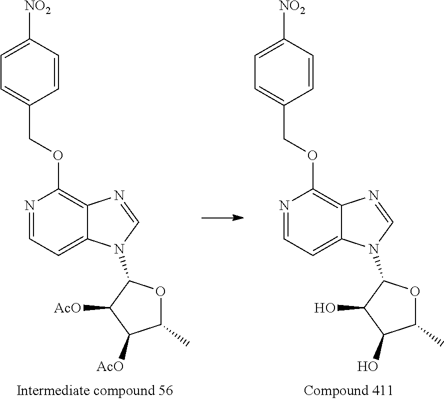 [SMILES: CC(=O)O[C@@H]1[C@H](OC(C)=O)[C@@H](C)O[C@H]1n1cnc2c(OCc3ccc([N+](=O)[O-])cc3)nccc21 ; C[C@H]1O[C@@H](n2cnc3c(OCc4ccc([N+](=O)[O-])cc4)nccc32)[C@H](O)[C@@H]1O]

To a solution of Intermediate Compound 56 (10 mg, 21.26 μmol, 1 eq) in MeOH (1 mL) was added NH3·H2O (910.00 mg, 7.27 mmol, 1 mL, 28% purity, 342.03 eq). The mixture was stirred at 25° C. for 1 hour. The reaction mixture was then filtered and concentrated under reduced pressure to give a residue. The residue was purified by Prep-HPLC (column: Phenomenex C18 75×30 mm×3 μm; mobile phase: [water(FA)-ACN]; B %: 18%-48%, 7 min) to Compound 411 (1.6 mg, 19% yield) as a white solid.

LCMS: Rt=0.747 min; (ESI positive ion) m/z: 387.0 (M+H)+(calculated: 387.12).

1H NMR (400 MHz, MeOD-d4) δ=8.37 (s, 1H), 8.25-8.17 (m, 2H), 7.54 (dd, J=7.9, 16.9 Hz, 3H), 6.78 (d, J=7.3 Hz, 1H), 6.45 (d, J=3.5 Hz, 1H), 5.40 (s, 2H), 4.38 (dd, J=3.7, 5.4 Hz, 1H), 4.14 (t, J=6.4 Hz, 1H), 3.90 (t, J=5.9 Hz, 1H), 1.44 (d, J=6.4 Hz, 3H).

### Compound 412:

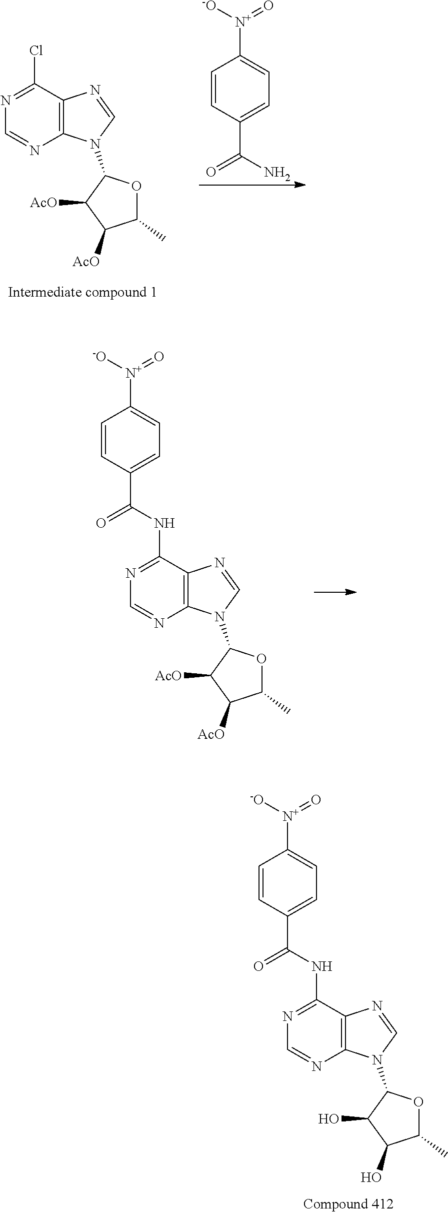

To a solution of Intermediate Compound 1 (100 mg, 281.89 μmol, 1 eq) in dioxane (3 mL) was added Cs2CO3 (183.69 mg, 563.79 μmol, 2 eq), 4-nitrobenzamide (60.88 mg, 366.46 μmol, 1.3 eq), and RuPhos Pd G4 (23.97 mg, 28.19 μmol, 0.1 eq). The mixture was stirred at 90° C. for 16 hr. The reaction mixture was then concentrated to give a crude product, which was purified by column chromatography (SiO2, Petroleum ether/Ethyl acetate=10/1 to 0/1) Then the residue was purified by Prep-TLC (SiO2, Petroleum ether:Ethyl acetate=1:1) to afford the corresponding amide compound (45 mg, crude) as a yellow oil.

LCMS: Rt=0.856 min; (ESI positive ion) m/z: 485.0 (M+H)+(calculated: 485.13).

To a solution of the above amide compound (38 mg, 78.44 μmol, 1 eq) in MeOH (4 mL) was added K2CO3 (16.26 mg, 117.67 μmol, 1.5 eq). The mixture was stirred at 0° C. for 0.5 hr. The reaction mixture was then concentrated to give a residue, which was purified by Prep-TLC (SiO2, DCM:MeOH=10:1). Then the residue was further purified by Prep-HPLC (column: Waters Xbridge 150×25 mm×5 μm; mobile phase: [water (NH4HCO3)-ACN]; B %: 13%-43%, 8 min) to give Compound 412 (4.22 mg, 13% yield) as a white solid.

LCMS: Rt=0.706 min; (ESI positive ion) m/z: 401.0 (M+H)+(calculated: 401.11).

1H NMR (400 MHz, MeOD-d4) δ=8.74 (s, 1H), 8.55 (s, 1H), 8.41 (d, J=8.8 Hz, 2H), 8.29 (d, J=8.8 Hz, 2H), 6.09 (d, J=4.4 Hz, 1H), 4.82 (br s, 1H), 4.20-4.12 (m, 2H), 1.45 (d, J=6.0 Hz, 3H).

### Compound 413:

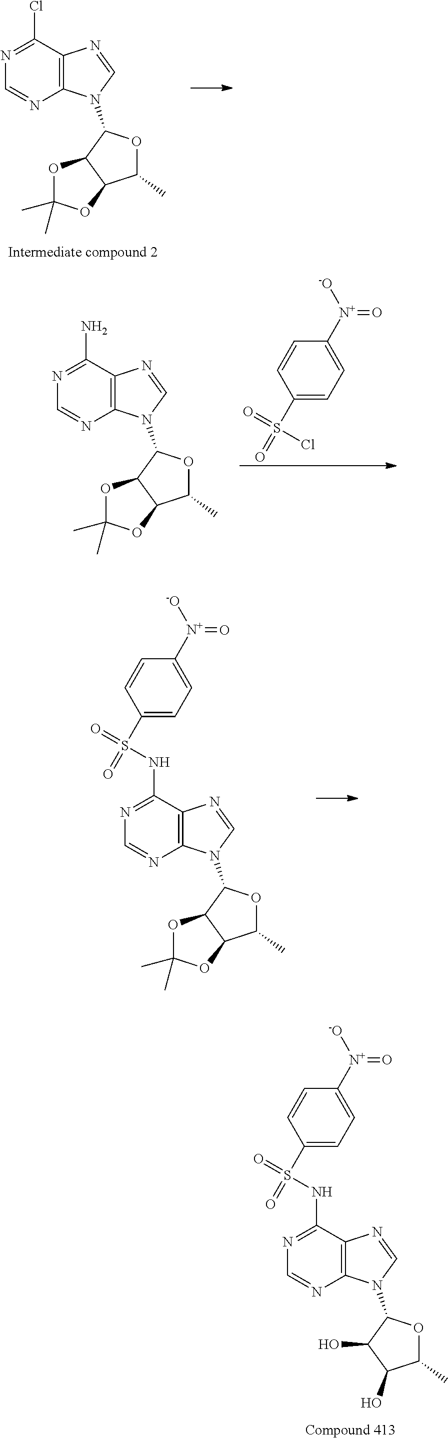

A solution of Intermediate Compound 2 (300 mg, 965.45 μmol, 1 eq) in NH3/MeOH (7 M, 3.00 mL, 21.75 eq) was stirred at 80° C. for 3 hr. The reaction mixture was concentrated to give a residue, which was purified by column chromatography (SiO2, Petroleum ether/Ethyl acetate=2/1 to 1/1) to afford the corresponding amine compound (200 mg, 71% yield) as a colorless gum.

LCMS: Rt=0.439 min; (ESI positive ion) m/z: 292.0 (M+H)+(calculated: 292.13).

A mixture of the above amine compound (200 mg, 686.57 μmol, 1 eq), 4-nitrobenzenesulfonyl chloride (197.80 mg, 892.54 μmol, 1.3 eq), pyridine (108.61 mg, 1.37 mmol, 110.83 μL, 2 eq) and 4-dimethylaminopyridine (167.75 mg, 1.37 mmol, 2 eq) in DCM (15 mL) was degassed and purged with N2 3 times. Then the mixture was stirred at 40° C. for 16 hr under N2 atmosphere. The reaction mixture was concentrated to give a residue. The residue was purified by Prep-HPLC (column: Unisil 3-100 C18 Ultra 150×50 mm×3 μm; mobile phase: [water(FA)-ACN]; B %: 26%-56%, 10 min) to give the corresponding sulfonamide compound (80 mg, 22% yield) as a white solid.

LCMS: Rt=0.856 min; (ESI positive ion) m/z: 477.0 (M+H)+(calculated: 477.11).

To a solution of the above sulfonamide compound (45 mg, 94.45 μmol, 1 eq) in water (0.2 mL) was added formic acid (976.00 mg, 21.21 mmol, 0.8 mL, 224.53 eq). The mixture was stirred at 25° C. for 0.5 hr. NH3·H2O was added to the reaction mixture at 0° C. until the pH reached 7. The mixture was then filtered and concentrated under reduced pressure to give a residue. The residue was purified by Prep-HPLC (column: Waters Xbridge 150×25 mm×5 μm; mobile phase: [water (NH4HCO3)-ACN]; B %: 1%-29%, 8 min) to give Compound 413 (6.5 mg, 15% yield) as a white solid.

LCMS: Rt=0.706 min; (ESI positive ion) m/z: 437.0 (M+H)+(calculated: 437.08).

1H NMR (400 MHz, MeOD-d4) δ=8.36-8.32 (m, 2H), 8.29-8.24 (m, 4H), 5.96 (d, J=4.3 Hz, 1H), 4.63 (t, J=4.8 Hz, 1H), 4.15-4.02 (m, 2H), 1.40 (d, J=6.4 Hz, 3H).

### Compound 415:

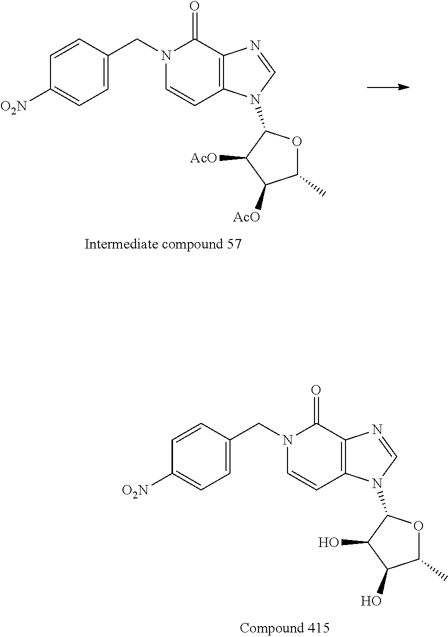 [SMILES: CC(=O)O[C@@H]1[C@H](OC(C)=O)[C@@H](C)O[C@H]1n1cnc2c(=O)n(Cc3ccc([N+](=O)[O-])cc3)ccc21 ; C[C@H]1O[C@@H](n2cnc3c(=O)n(Cc4ccc([N+](=O)[O-])cc4)ccc32)[C@H](O)[C@@H]1O]

Compound 415 (3.26 mg, 39%) was synthesized from Intermediate Compound 57 following the protocol described for Compound 411.

LCMS: Rt=0.728 min; (ESI positive ion) m/z: 387.0 (M+H)+(calculated: 387.12).

1H NMR (400 MHz, MeOD-d4) δ=8.27-8.17 (m, 3H), 7.63 (d, J=7.3 Hz, 1H), 7.53 (d, J=8.7 Hz, 2H), 6.85 (d, J=7.3 Hz, 1H), 5.84 (d, J=5.5 Hz, 1H), 5.44 (s, 2H), 4.40 (t, J=5.4 Hz, 1H), 4.19 (dd, J=4.4, 6.5 Hz, 1H), 4.02-3.92 (m, 1H), 1.45 (d, J=6.6 Hz, 3H).

### Compound 416:

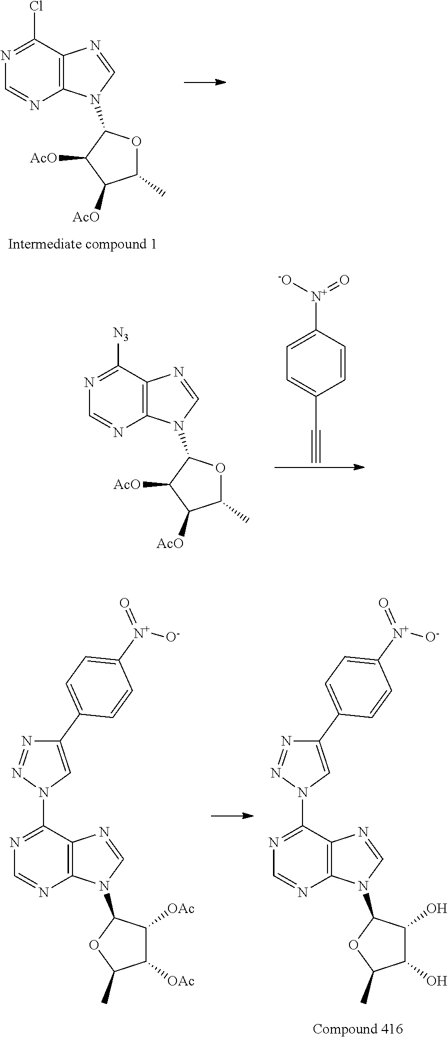

To a solution of Intermediate Compound 1 (300 mg, 845.68 μmol, 1 eq) in DMF (3 mL) was added sodium azide (109.95 mg, 1.69 mmol, 2 eq). The mixture was stirred at 20° C. for 16 hr. The mixture was diluted with water (20 mL) and adjusted to a pH of about 10. The mixture was extracted with ethyl acetate (20 mL×2). The combined organic layers were washed with brine (20 mL×2), dried over Na2SO4, filtered and concentrated under reduced pressure to give the corresponding azide compound (300 mg, 98% yield) as a yellow oil.

To a solution of the above azide compound (240 mg, 664.25 μmol, 1 eq) and 1-ethynyl-4-nitro-benzene (97.73 mg, 664.25 μmol, 1 eq) in DCM (8 mL) and water (8 mL) were added copper sulfate pentahydrate (16.59 mg, 66.42 μmol, 0.1 eq) and sodiμm; (2R)-2-[(1S)-1,2-dihydroxyethyl]-4-hydroxy-5-oxo-2H-furan-3-olate (26.32 mg, 132.85 μmol, 0.2 eq). The mixture was stirred at 20° C. for 16 hr. The mixture was diluted with water (30 mL) and extracted with DCM (20 mL×2). The combined organic layers were washed with brine (20 mL×2), dried over Na2SO4, filtered and concentrated under reduced pressure to give a residue. The residue was purified by column chromatography (SiO2, petroleum ether/ethyl acetate=100/0 to 1/1; TLC petroleum ether/ethyl acetate=1/1, Rf=0.5) to give the corresponding triazole compound (0.3 g, 89% yield) as a brown solid.

1H NMR (400 MHz, DMSO-d6) δ=9.89 (s, 1H), 9.11 (s, 1H), 9.09 (s, 1H), 8.38 (s, 4H), 6.39 (d, J=5.2 Hz, 1H), 6.08 (t, J=5.6 Hz, 1H), 5.44 (t, J=5.6 Hz, 1H), 4.33 (q, J=6.0 Hz, 1H), 2.14 (s, 3H), 2.05 (s, 3H), 1.46 (d, J=6.4 Hz, 3H).

To a solution of the above triazole compound (0.3 g, 590.04 μmol, 1 eq) in MeOH (6 mL) was added NH3·H2O (5.46 g, 43.62 mmol, 6 mL, 28% purity, 73.93 eq). The mixture was stirred at 50° C. for 2 hr. The mixture was concentrated to give a residue. The residue was purified by Prep-HPLC (column: Waters Xbridge C18 150×50 mm×10 μm; mobile phase: [water (NH4HCO3)-ACN]; B %: 22%-52%, 11 min) to give Compound 416 (54.9 mg, 21% yield) as a white solid.

LCMS: Rt=0.829 min; (ESI positive ion) m/z: 425.0 (M+H)+(calculated: 425.12).

1H NMR (400 MHz, DMSO-d6) δ=9.90 (s, 1H), 9.08 (s, 1H), 9.05 (s, 1H), 8.39 (s, 4H), 6.10 (d, J=4.8 Hz, 1H), 5.59 (d, J=5.6 Hz, 1H), 5.29 (d, J=5.2 Hz, 1H), 4.77 (q, J=5.2 Hz, 1H), 4.12-4.02 (m, 2H), 1.42-1.33 (m, 3H).

### Compound 417:

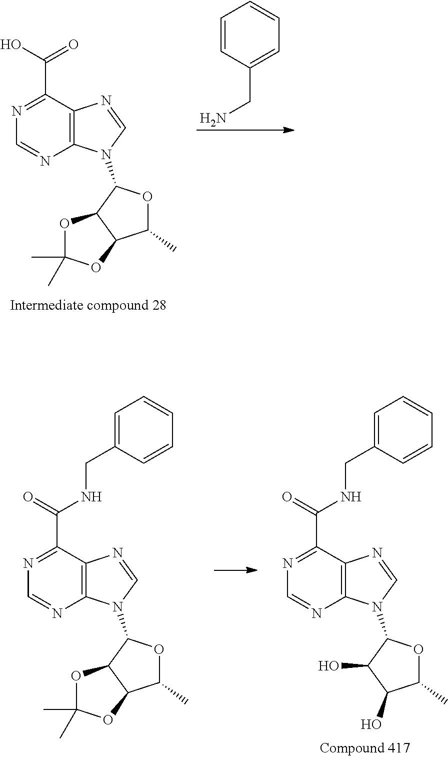 [SMILES: C[C@H]1O[C@@H](n2cnc3c(C(=O)O)ncnc32)[C@@H]2OC(C)(C)O[C@@H]21 ; NCc1ccccc1 ; C[C@H]1O[C@@H](n2cnc3c(C(=O)NCc4ccccc4)ncnc32)[C@@H]2OC(C)(C)O[C@@H]21 ; C[C@H]1O[C@@H](n2cnc3c(C(=O)NCc4ccccc4)ncnc32)[C@H](O)[C@@H]1O]

To a mixture of Intermediate Compound 28 (100 mg, 312.21 μmol, 1 eq) and phenylmethanamine (40.14 mg, 374.65 μmol, 40.84 μL, 1.2 eq) in THE (10 mL) was added DIEA (201.75 mg, 1.56 mmol, 271.90 μL, 5 eq) and T3P (496.69 mg, 780.52 μmol, 464.20 μL, 50% purity, 2.5 eq) at 0° C. The mixture was stirred at 25° C. for 1 hr. The mixture was concentrated under vacuum and the residue was purified by Prep-TLC (Petroleum ether/Ethyl acetate=1/2) to afford the corresponding amide compound (90 mg, 70% yield) as a colorless oil.

A solution of the above amide compound (65 mg, 158.75 μmol, 1 eq) in formic acid (2.4 mL) and water (0.6 mL) was stirred at 0° C. for 6 hr. The mixture was adjusted to a pH of 6 using NH3·H2O. The mixture was concentrated under vacuum and the residue was purified by Prep-HPLC (column: Waters xbridge 150×25 mm 10 μm; mobile phase: [water(NH4HCO3)-ACN]; B %: 11%-41%, 8 min) to afford Compound 417 (9.42 mg, 9% yield) as a white solid. LCMS: Rt=0.398 min; (ESI positive ion) m/z: 370.2 (M+H)+(calculated: 370.14).

1H NMR (400 MHz, DMSO-d6) δ=9.46 (br t, J=6.4 Hz, 1H), 9.07 (s, 1H), 8.95 (s, 1H), 7.41-7.38 (m, 2H), 7.37-7.32 (m, 2H), 7.29-7.24 (m, 1H), 6.03 (d, J=4.8 Hz, 1H), 5.55 (d, J=5.6 Hz, 1H), 5.26 (d, J=5.2 Hz, 1H), 4.72 (q, J=5.0 Hz, 1H), 4.59 (d, J=6.2 Hz, 2H), 4.06-4.00 (m, 2H), 1.34 (d, J=6.2 Hz, 3H).

### Compound 418:

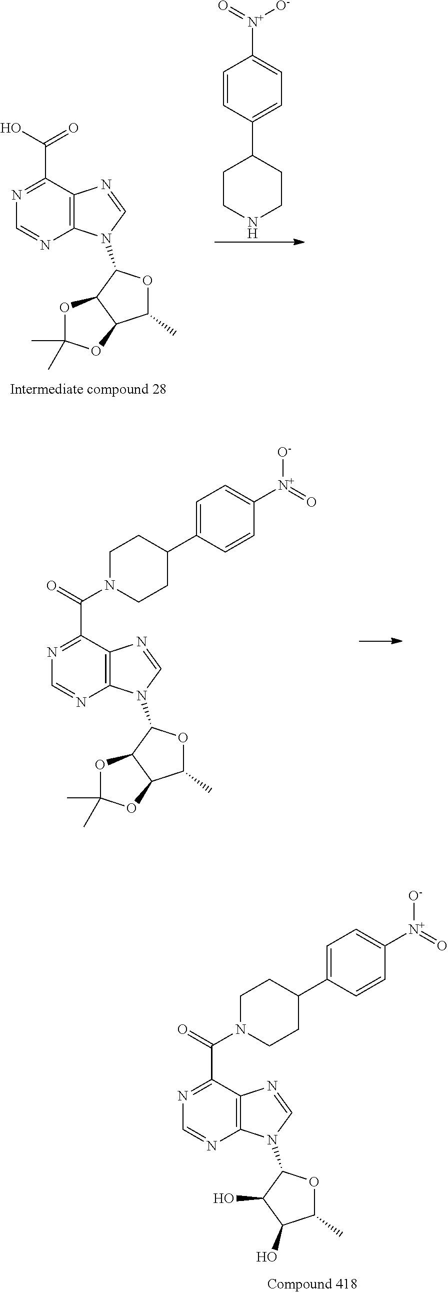 [SMILES: C[C@H]1O[C@@H](n2cnc3c(C(=O)O)ncnc32)[C@@H]2OC(C)(C)O[C@@H]21 ; O=[N+]([O-])c1ccc(C2CCNCC2)cc1 ; C[C@H]1O[C@@H](n2cnc3c(C(=O)N4CCC(c5ccc([N+](=O)[O-])cc5)CC4)ncnc32)[C@@H]2OC(C)(C)O[C@@H]21 ; C[C@H]1O[C@@H](n2cnc3c(C(=O)N4CCC(c5ccc([N+](=O)[O-])cc5)CC4)ncnc32)[C@H](O)[C@@H]1O]

To a solution of Intermediate Compound 28 (100 mg, 312.21 μmol, 1 eq) and 4-(4-nitrophenyl)piperidine (77.27 mg, 374.65 μmol, 1.2 eq) in THF (10 mL) was added T3P (496.69 mg, 780.52 μmol, 464.20 μL, 50% purity, 2.5 eq) and DIEA (201.75 mg, 1.56 mmol, 271.90 μL, 5 eq) at 0° C. The mixture was stirred at 25° C. for 1 hr. The mixture was filtered and concentrated under reduced pressure to give a residue, which was purified by Prep-TLC (SiO2, Petroleum ether:Ethyl acetate=0:1) to afford the corresponding amide compound (131 mg, 80% yield) as a white solid.

LCMS: Rt=0.437 min; (ESI positive ion) m/z: 509.2 (M+H)+(calculated: 509.21).

A solution of above amide compound (131 mg, 257.61 μmol, 1 eq) in water (1 mL) and formic acid (4 mL) was stirred at 0° C. for 6 hr. NH3·H2O was added to the reaction mixture at 0° C. until a pH of 7 was reached. The mixture was then filtered and concentrated under reduced pressure to give a residue, which was dissolved in MeOH (2 mL) and then purified by Prep-HPLC (column: Waters xbridge 150×25 mm 10 μm; mobile phase: [water (NH4HCO3)-ACN]; B %: 16%-46%, 11 min) to afford Compound 418 (52 mg, 43% yield) as a white solid.

LCMS: Rt=0.340 min; (ESI positive ion) m/z: 469.2 (M+H)+(calculated: 469.18).

1H NMR (400 MHz, DMSO-d6) δ=9.01 (s, 1H), 8.88 (s, 1H), 8.19 (d, J=8.6 Hz, 2H), 7.57 (d, J=8.8 Hz, 2H), 6.02 (d, J=5.0 Hz, 1H), 5.52 (dd, J=2.2, 5.6 Hz, 1H), 5.24 (d, J=5.0 Hz, 1H), 4.77-4.74 (m, 1H), 4.07-3.99 (m, 2H), 3.43 (br d, J=13.4 Hz, 1H), 3.30 (br s, 1H), 3.25-3.16 (m, 1H), 3.12-2.95 (m, 2H), 2.00 (br d, J=13.2 Hz, 1H), 1.76-1.62 (m, 3H), 1.34 (d, J=5.8 Hz, 3H).

### Compound 419:

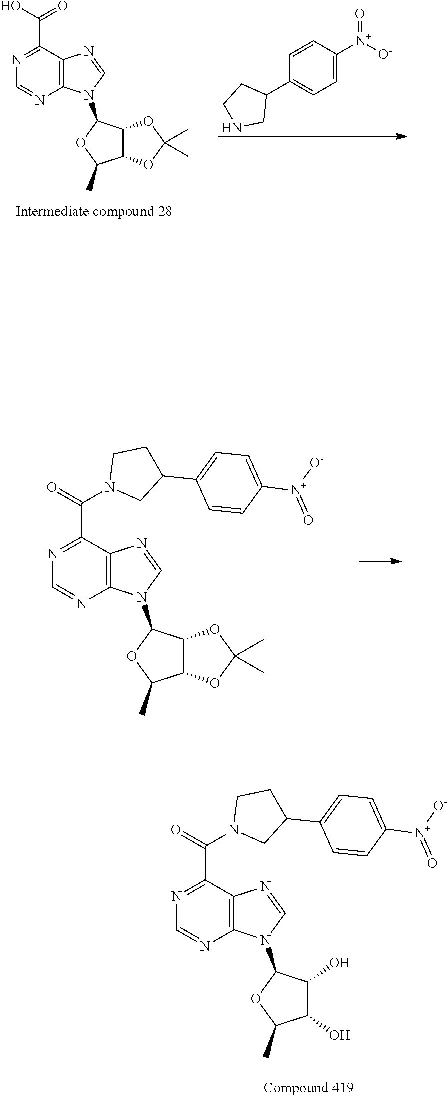

Compound 419 (66.5 mg, 72% yield) was synthesized from Intermediate Compound 28 and 3-(4-nitrophenyl)pyrrolidine following the protocols described for Compound 360.

LCMS: Rt=0.802 min; (ESI positive ion) m/z: 455.0 (M+H)+(calculated: 455.16).

1H NMR (400 MHz, DMSO-d6) δ=9.02 (d, J=13.2 Hz, 1H), 8.89 (d, J=13.6 Hz, 1H), 8.22 (d, J=8.8 Hz, 1H), 8.13 (d, J=8.8 Hz, 1H), 7.69 (d, J=8.8 Hz, 1H), 7.56 (d, J=8.8 Hz, 1H), 6.02 (dd, J=5.2, 10.0 Hz, 1H), 5.51 (dd, J=5.6, 11.6 Hz, 1H), 5.23 (dd, J=5.2, 6.8 Hz, 1H), 4.83-4.67 (m, 1H), 4.23-3.37 (m, 7H), 2.67 (br d, J=2.0 Hz, 1H), 2.43-2.28 (m, 1H), 2.18-2.02 (m, 1H), 1.34 (t, J=6.8 Hz, 3H).

### Compound 420:

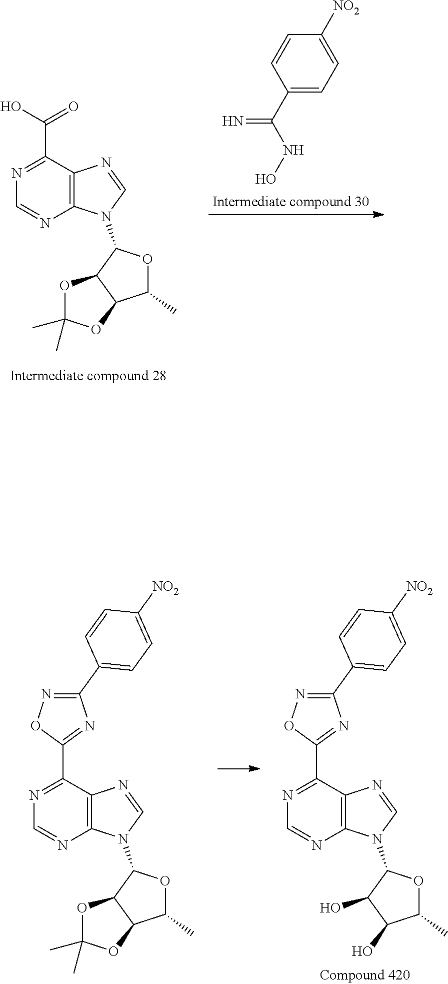 [SMILES: C[C@H]1O[C@@H](n2cnc3c(C(=O)O)ncnc32)[C@@H]2OC(C)(C)O[C@@H]21 ; N=C(NO)c1ccc([N+](=O)[O-])cc1 ; C[C@H]1O[C@@H](n2cnc3c(-c4nc(-c5ccc([N+](=O)[O-])cc5)no4)ncnc32)[C@@H]2OC(C)(C)O[C@@H]21 ; C[C@H]1O[C@@H](n2cnc3c(-c4nc(-c5ccc([N+](=O)[O-])cc5)no4)ncnc32)[C@H](O)[C@@H]1O]

To a solution of Intermediate Compound 28 (100 mg, 312.21 μmol) in MeCN (5 mL) was added carbonyldiimidazole (57 mg, 351.53 μmol). The mixture was stirred at 25° C. for 30 minutes. Intermediate Compound 30 (58 mg, 320.18 μmol) was added to the mixture and the reaction mixture was stirred at 25° C. for an additional 2 hr. The mixture was heated to 85° C. and stirred for 9.5 hr. The reaction mixture was extracted with ethyl acetate, filtered and concentrated under vacuum to afford the corresponding oxadiazole compound (90 mg, 61% yield) as a white solid.

LCMS: Rt=0.742 min; (ESI positive ion) m/z: 466.2 (M+H)+(calculated: 466.14).

To a solution of the above oxadiazole compound (80 mg, 171.89 μmol) in formic acid (3.2 mL) and water (0.8 mL) was stirred at 0° C. for 3.5 hr. NH3·H2O was added to the reaction mixture at 0° C. until the pH reached 7. The reaction was then filtered and concentrated under reduced pressure to give a residue. The residue was dissolved in DMSO (2 mL) and purified by Prep-HPLC (column: Phenomenex C18 150×25 mm×10 μm; mobile phase: [water(NH4HCO3)-ACN]; B %: 20%-50%, 5 min) to afford Compound 420 (4.21 mg, 6% yield) as a white solid.

LCMS: Rt=0.445 min; (ESI positive ion) m/z: 426.1 (M+H)+(calculated: 426.11).

1H NMR (400 MHz, DMSO-d6) δ=9.26 (s, 1H), 9.12 (s, 1H), 8.51-8.44 (m, 3H), 8.51-8.43 (m, 1H), 6.10 (d, J=5.2 Hz, 1H), 5.58 (d, J=5.6 Hz, 1H), 5.28 (d, J=5.4 Hz, 1H), 4.81-4.73 (m, 1H), 4.10-4.04 (m, 2H), 1.38 (d, J=6.0 Hz, 3H)).

### Compound 421:

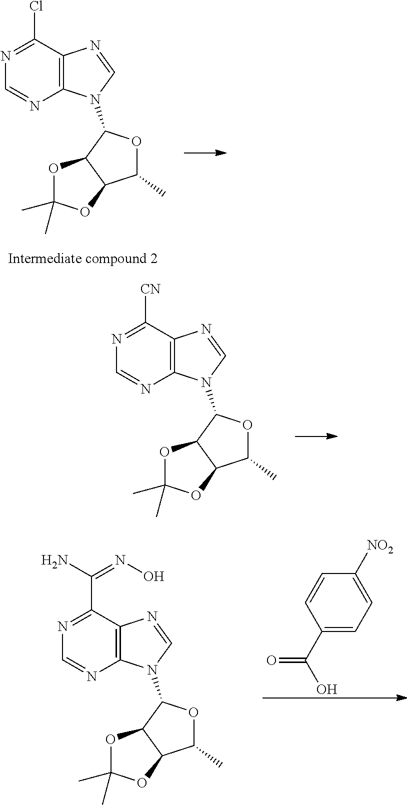 [SMILES: C[C@H]1O[C@@H](n2cnc3c(Cl)ncnc32)[C@@H]2OC(C)(C)O[C@@H]21 ; C[C@H]1O[C@@H](n2cnc3c(C#N)ncnc32)[C@@H]2OC(C)(C)O[C@@H]21 ; C[C@H]1O[C@@H](n2cnc3c(/C(N)=N\O)ncnc32)[C@@H]2OC(C)(C)O[C@@H]21 ; O=C(O)c1ccc([N+](=O)[O-])cc1]

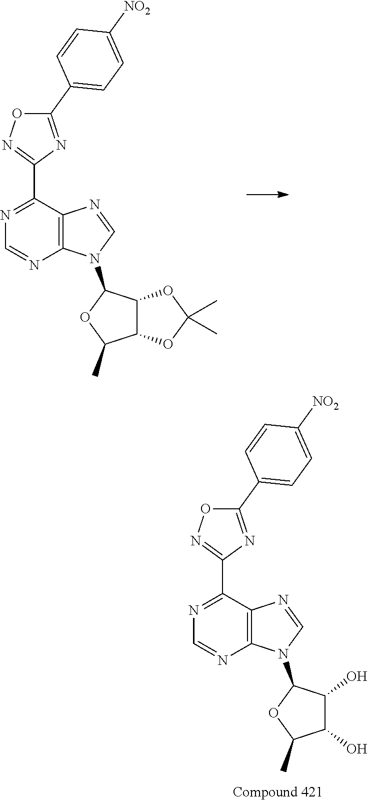 [SMILES: C[C@H]1O[C@@H](n2cnc3c(-c4noc(-c5ccc([N+](=O)[O-])cc5)n4)ncnc32)[C@@H]2OC(C)(C)O[C@@H]21 ; C[C@H]1O[C@@H](n2cnc3c(-c4noc(-c5ccc([N+](=O)[O-])cc5)n4)ncnc32)[C@H](O)[C@@H]1O]

To a mixture of Intermediate Compound 2 (500 mg, 1.61 mmol, 1 eq), 1,1′-bis(diphenylphosphino)ferrocene (178.41 mg, 321.82 μmol, 0.2 eq) and Pd2(dba)3 (147.35 mg, 160.91 μmol, 0.1 eq) in DMF (8 mL) was added zinc cyanide (113.37 mg, 965.45 μmol, 61.28 μL, 0.6 eq) and zinc (21.04 mg, 321.82 μmol, 0.2 eq) under N2. The mixture was stirred at 120° C. for 16 hr under N2 atmosphere. The mixture was poured into water (50 mL). The aqueous phase was extracted with ethyl acetate (20 mL×2). The combined organic phase was washed with brine (50 mL), dried with anhydrous Na2SO4, filtered and concentrated under vacuum. The residue was purified by column chromatography (SiO2, Petroleum ether/Ethyl acetate=100/1 to 3/1) to afford the corresponding nitrile compound (150 mg, 31% yield) as a yellow oil.

To a solution of the above nitrile compound (140 mg, 464.65 μmol, 1 eq) in EtOH (5 mL) and water (2 mL) was added hydroxylamine hydrochloride (129.16 mg, 1.86 mmol, 4 eq) and Na2CO3 (147.74 mg, 1.39 mmol, 3 eq). The mixture was stirred at 85° C. for 2 hr. The mixture was then poured into water (30 mL). The aqueous phase was extracted with ethyl acetate (30 mL×2). The combined organic phase was washed with brine (10 mL), dried with anhydrous Na2SO4, filtered and concentrated under reduced pressure to afford the corresponding N-hydroxyimidamide compound (138 mg, 89% yield) as a yellow oil.

LCMS: Rt=0.268 min; (ESI positive ion) m/z: 335.1 (M+H)+(calculated: 335.14).

To a solution of the 4-nitrobenzoic acid (82.78 mg, 495.32 μmol, 1.2 eq) in MeCN (5 mL), the carbonyldiimidazole (80.32 mg, 495.32 μmol, 1.2 eq) was added, and the mixture was stirred at 25° C. for 0.5 hr. The above N-hydroxyimidamide compound (138 mg, 412.77 μmol, 1 eq) was then added and the mixture was stirred 25° C. for 12 hr. And then the mixture was stirred at 85° C. for 2 hr. The reaction mixture was extracted with ethyl acetate (20 mL×2). The combined organic layers were dried over Na2SO4, filtered and concentrated under reduced pressure to give a residue, which was purified by re-crystallization from methanol (1.0 mL) at 25° C. to afford the corresponding oxadiazole compound (53 mg, 27% yield) as a brown solid.

LCMS: Rt=0.555 min; (ESI positive ion) m/z: 466.1 (M+H)+(calculated: 466.14).

A mixture of the above oxadiazole compound (53 mg, 113.88 μmol, 1 eq) in formic acid (0.8 mL) and water (0.2 mL) was degassed and purged with N2 3 times. The mixture was stirred at 0° C. for 8 hr under N2 atmosphere. NH3·H2O was added to the reaction mixture at 0° C. until a pH of 7 was achieved. The mixture was then concentrated under reduced pressure to give a residue, which was purified by Prep-HPLC (column: Phenomenex C18 150×25 mm×10 μm; mobile phase: [water (NH4HCO3)-ACN]; B %: 11%-41%, 8 min) to afford Compound 421 (3.6 mg, 7% yield) as a yellow solid.

LCMS: Rt=0.428 min; (ESI positive ion) m/z: 426.0 (M+H)+(calculated: 426.11).

1H NMR (400 MHz, DMSO-d6) δ=9.21 (s, 1H), 9.02 (s, 1H), 8.51 (s, 4H), 6.09 (d, J=4.8 Hz, 1H), 5.57 (d, J=5.6 Hz, 1H), 5.27 (d, J=5.0 Hz, 1H), 4.79-4.73 (m, 1H), 4.09-4.04 (m, 2H), 1.37 (d, J=6.0 Hz, 3H).

### Compound 422:

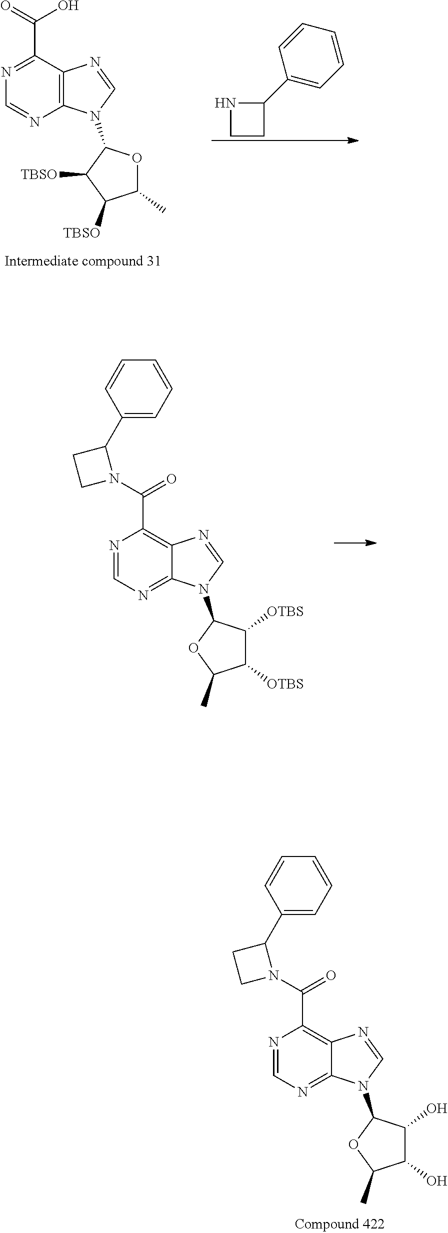 [SMILES: C[C@H]1O[C@@H](n2cnc3c(C(=O)O)ncnc32)[C@H](O[Si](C)(C)C(C)(C)C)[C@@H]1O[Si](C)(C)C(C)(C)C ; c1ccc(C2CCN2)cc1 ; C[C@H]1O[C@@H](n2cnc3c(C(=O)N4CCC4c4ccccc4)ncnc32)[C@H](O[Si](C)(C)C(C)(C)C)[C@@H]1O[Si](C)(C)C(C)(C)C ; C[C@H]1O[C@@H](n2cnc3c(C(=O)N4CCC4c4ccccc4)ncnc32)[C@H](O)[C@@H]1O]

To a solution of Intermediate Compound 31 (20 mg, 30.08 μmol, 76.52% purity) in THE (0.2 mL) was added 2-phenylazetidine (4.01 mg, 30.08 μmol), T3P (47.86 mg, 75.20 mol, 44.73 μL, 50% purity) and DIEA (19.44 mg, 150.41 μmol, 26.20 μL) at 0° C. The mixture was stirred at 25° C. for 1 hr. The residue was purified by Prep-TLC (SiO2, Petroleum ether:Ethyl acetate=1:1) to afford the corresponding amide compound (19 mg, 98% yield) as a white solid. LCMS: Rt=0.838 min; (ESI positive ion) m/z: 624.2 (M+H)+(calculated: 624.33).

To a solution of the above amide compound (14 mg, 22.44 μmol) in MeOH (0.2 mL) was added NH4F (24.37 mg, 658.12 μmol). The mixture was stirred at 65° C. for 7 hr. The reaction mixture was then filtered and concentrated under reduced pressure to give a residue, which was purified by Prep-HPLC (column: Phenomenex C18 150×25 mm×10 μm; mobile phase: [water (NH4HCO3)-ACN]; B %: 5%-35%, 8 min) to afford the Compound 422 as a white solid.

LCMS: Rt=0.344 min; (ESI positive ion) m/z: 396.2 (M+H)+(calculated: 396.16).

1H NMR (400 MHz, DMSO-d6) δ=9.05 (s, 1H), 8.94 (d, J=1.1 Hz, 1H), 8.75 (d, J=2.8 Hz, 1H), 8.72 (s, 1H), 7.54-7.52 (m, 2H), 7.45-7.41 (m, 2H), 7.34-7.30 (m, 1H), 7.00-6.96 (m, 2H), 6.93-6.88 (m, 2H), 6.03 (d, J=4.6 Hz, 1H), 5.89 (d, J=4.5 Hz, 1H), 5.63-5.48 (m, 4H), 5.27-5.19 (m, 2H), 4.77-4.71 (m, 1H), 4.59-4.54 (m, 1H), 4.37-4.30 (m, 2H), 4.27-4.19 (m, 1H), 4.16-4.10 (m, 1H), 4.06-3.95 (m, 4H), 2.83-2.78 (m, 2H), 2.10 (br dd, J=5.2, 10.2 Hz, 2H), 1.35 (d, J=6.0 Hz, 3H), 1.31 (d, J=6.0 Hz, 2H).

### General Procedure 1: Deprotection of Acetonides

A solution of an acetonide (1 eq) in HCOOH/H2O=4/1 (V/V) (2 mL) was stirred at 0° C. for 7 hr. The reaction mixture was adjusted to a pH of 7 to 8 by addition of a solution of ammonia at 0° C. The mixture was diluted by addition of water (10 mL) and extracted with EtOAc (20 mL×3). The organic layer was washed with brine, dried by Na2SO4, and concentrated to give a residue. The residue was purified by Prep-HPLC (column: Waters xbridge 150×25 mm×10 μm; mobile phase: [water (NH4HCO3)-ACN]; B %: 28%-58%, 8 min). The purified solution was lyophilized to afford the corresponding diol.

General Procedure 2: Deprotection of t-Butyldimethylsilyl Ethers

To a solution of t-butyldimethylsilyl ether (1 eq) in MeOH (0.5 mL/20 μmmol) was added NH4F (12.31 mg, 332.42 μmol, 20 eq). The mixture was stirred at 65° C. for 12 hr. The reaction mixture concentrated under reduced pressure to give a residue, which was purified by Prep-HPLC (column: Phenomenex C18 150×25 mm×10 μm; mobile phase: [water(NH4HCO3)-ACN]; B %: 31%-61%, 8 min) to afford the corresponding diol.

### General Procedure 3: Coupling of Amine to Intermediate Compounds 3, 4, 5, 7, 9, 18, and 19

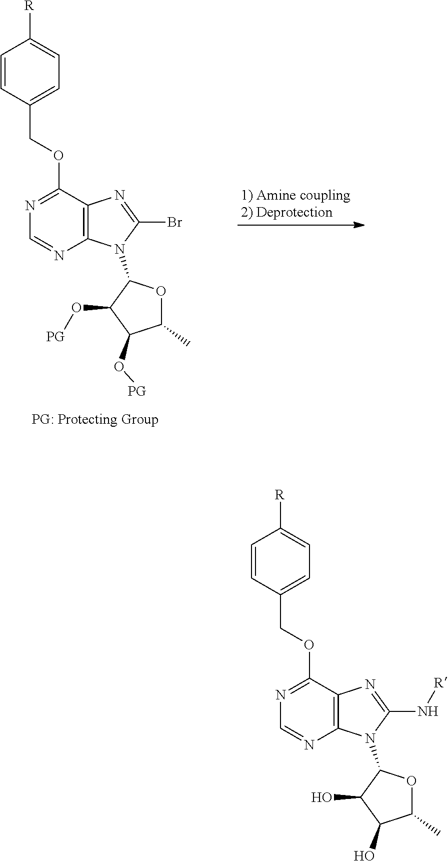

Step 1) To a solution of the chosen intermediate compound (197.51 μmol, 1 eq) in DMSO (5 mL) was added the desired amine (592.53 μmol, 3 eq) and TEA (99.93 mg, 987.55 mol, 137.45 μL, 5 eq). The mixture was stirred at 100° C. for 2 hr. The reaction mixture was diluted with water (10 mL) and extracted with ethyl acetate (20 mL×2). The combined organic layers were washed with brine, dried over Na2SO4, filtered, and concentrated under reduced pressure to give a residue. The residue was purified by preparative-TLC (SiO2, petrol ether/ethyl acetate) to afford the coupled amine product.

Step 2) For compounds starting from Intermediate Compounds 3, 4, 5, and 18, the final amine compound was obtained after deprotection of the acetonide compound prepared in Step 1 by following General Procedure 1. For compounds starting from Intermediate Compounds 7, 9, and 19, the final amine compound was obtained after deprotection of the t-butyldimethylsilyl ether compound prepared in Step 1 following General Procedure 2.

Compounds prepared by this general method are shown in Table 3. The diastereomers corresponding to Compound 137 and Compound 141 were separated after Step 1 by TLC (SiO2, Petroleum ether:Ethyl acetate, for example 2:1) to give each diastereomer, which was then carried through Step 2.

### General Procedure 4: Coupling of Amine to Intermediate Compounds 6 and 10

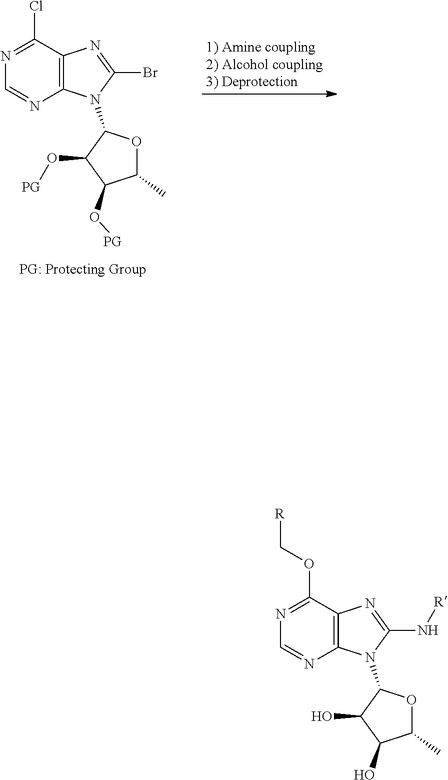

Step 1) To a mixture of Intermediate Compound 6 or 10 (1.03 mmol, 1 eq) in EtOH (8 mL) was added the desired amine (1.03 mmol, 1 eq) and TEA (312.68 mg, 3.09 mmol, 430.09 μL, 3 eq). The mixture was stirred at 80° C. for 1 hr and then concentrated under vacuum. The resultant residue was purified by Prep-TLC (SiO2, Petroleum ether:Ethyl acetate) to give the coupled amine product.

Step 2) To a solution of the preceding amine compound (240.49 μmol, 1 eq) in toluene (5 mL) was added Xantphos (27.83 mg, 48.10 μmol, 0.2 eq) and Cs2CO3 (195.89 mg, 601.22 μmol, 2.5 eq), Pd2(dba)3 (22.02 mg, 24.05 μmol, 0.1 eq), and the desired alcohol (240.49 mol, 1 eq). The mixture was degassed, purged with N2 3 times, and stirred at 80° C. for 12 hr under N2 atmosphere. The mixture was concentrated under vacuum and the resultant residue was purified by Prep-TLC (SiO2, Petroleum ether:Ethyl acetate=1:1) to afford the alcohol coupled compound.

Step 3) For compounds starting from Intermediate Compound 6, the final amine compound was obtained after deprotection of the acetonide compound prepared in Step 2 by following General Procedure 1. For compounds starting from Intermediate Compound 10, the final amine compound was obtained after deprotection of the t-butyldimethylsilyl ether compound prepared in Step 2 following General Procedure 2.

Compounds prepared by this general method are shown in Table 2. The following pairs of diastereomers were separated by TLC (SiO2, Petroleum ether:Ethyl acetate=1:1) after Step 2 to give each diastereomer, which was then carried through Step 3: (1) Compound 117 and Compound 118; (2) Compound 119 and Compound 120; (3) Compound 121 and Compound 122; (4) Compound 123 and Compound 124; (5) Compound 125 and Compound 126; (6) Compound 127 and Compound 128; (7) Compound 129 and Compound 130; (8) Compound 131 and Compound 132; and (9) Compound 133 and Compound 134.

### General Procedure 5: Coupling of Alcohol to Intermediate Compound 1

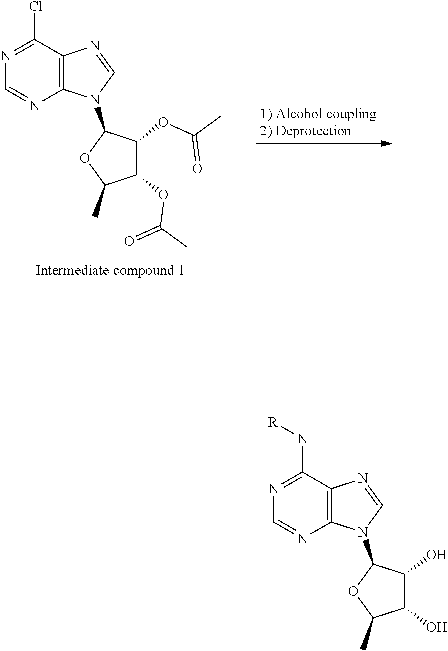

Step 1) A mixture of Intermediate Compound 1 (281.89 μmol, 1 eq), the desired alcohol (563.79 μmol, 2 eq), Cs2CO3 (704.73 μmol, 2.5 eq), Xantphos (56.38 μmol, 0.2 eq), and Pd2(dba)3 (28.19 μmol, 0.1 eq) in toluene (5 mL) was degassed and purged with N2 3 times. The mixture was stirred at 80° C. for 0.5 hr under N2 atmosphere. The mixture was then filtered and concentrated under vacuum. The residue was purified by column chromatography (SiO2, DCM/MeOH=10/1) to afford the alcohol coupled compound.

Step 2) The alcohol coupled compound from Step 1 was used directly in a deprotection step following General Procedure 6.

Compounds prepared by this general method are shown in Table 5.

### General Procedure 6: Deprotection of Diacetate

A solution of diacetate compound (302.84 μmol, crude) in MeOH/NH3·H2O (v/v=1/1) was stirred at 50° C. for 1 hr. The reaction mixture was adjusted to a pH of about 7 by a solution of HCl (1 N). Then the mixture was filtered and concentrated under vacuum. The residue was purified by Prep-HPLC (column: Waters Xbridge C18 150×50 mm×10 μm; mobile phase: [water (NH4HCO3)-ACN]) to afford the corresponding diol.

### General Procedure 7: Coupling of Amine to Intermediate Compound 1

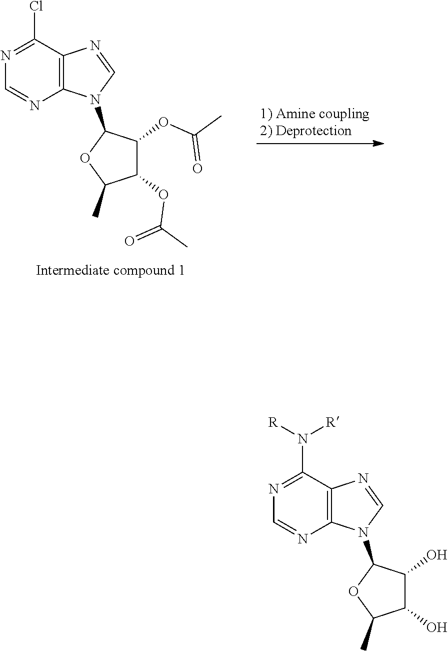

Step 1) To a mixture of Intermediate Compound 1 (197.32 μmol, 1 eq) and TEA (1.58 mmol, 8 eq) in EtOH (4 mL) was added the desired amine (394.65 μmol, 2 eq). The mixture was stirred at 80° C. for 2 hr. The mixture was concentrated under vacuum to afford the amine coupled compound.

Step 2) The amine coupled compound from Step 1 was used directly in a deprotection step following General Protocol 6.

Compounds prepared by this general method are shown in Table 4.

### General Procedure 8: Coupling of Thiol to Intermediate Compound 1

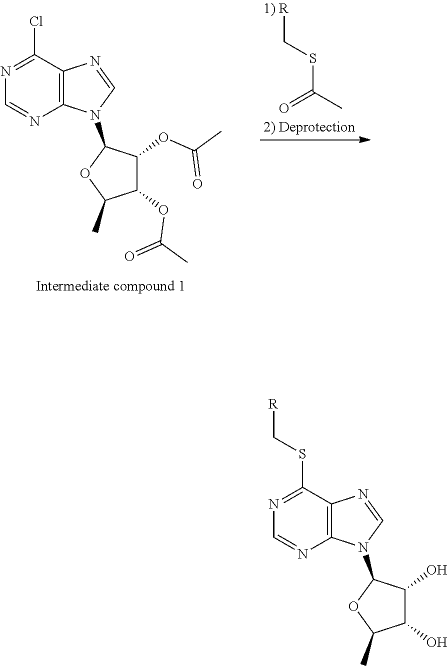

Step 1) To a solution of Intermediate Compound 1 (281.89 μmol, 1 eq) and the corresponding S-alkyl ethanethioate (338.27 μmol, 1.2 eq) in MeOH (0.8 mL) and THE (0.2 mL) was added K2CO3 (563.79 μmol, 2 eq) in one portion at 0° C. The mixture was stirred at 0° C. for 2 hours. The mixture was poured into water (10 mL) and the aqueous phase was extracted with ethyl acetate (10 mL×2). The combined organic phase was washed with brine (5 mL), dried with anhydrous Na2SO4, filtered and concentrated under vacuum. The resultant residue was purified by Prep-HPLC (column: Waters Xbridge C18 150×50 mm×10 μm; mobile phase: [water (NH4HCO3)-ACN]) to afford the protected thioether compound.

Step 2) The thioether compound from Step 1 was used directly in a deprotection step following General Protocol 6.

Compounds prepared by this general method are shown in Table 6.

Additional analytical data of each compound shown in Tables 2-6 are summarized in Table 7.

TABLE 2

| Comp. | | Intermediate | | | General | LCMS | LCMS | Calculated |
|---|---|---|---|---|---|---|---|---|
| No. | Structure | compound | Amine | Alcohol | procedure | Rt (min) | (M + H)+ | (M + H)+ |
| 14 | 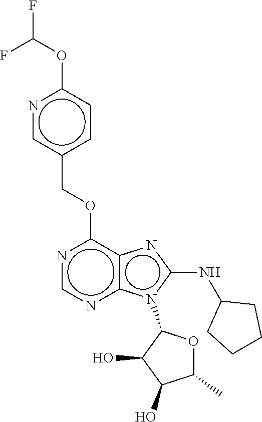 | 6 | cyclopentanamine | (6-(difluoromethoxy)pyridin- 3-yl)methanol | 4 | 0.483 | 493.3 | 493.19 |
| 15 | 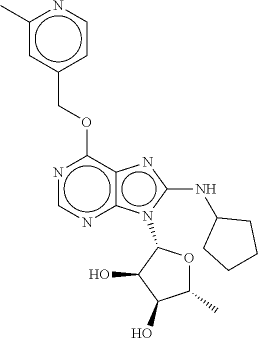 | 6 | cyclopentanamine | (2-methylpyridin-4- yl)methanol | 4 | 0.354 | 441.1 | 441.22 |
| 37 | 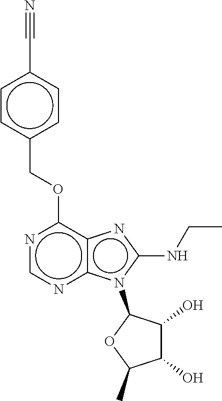 | 6 | ethanamine | 4- (hydroxymethyl)benzonitrile | 4 | 0.410 | 411.1 | 411.17 |
| 39 | 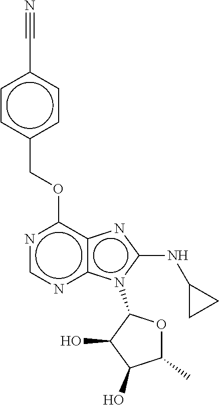 | 6 | cyclopropanamine | 4- (hydroxymethyl)benzonitrile | 4 | 0.352 | 423.1 | 423.17 |
| 40 | 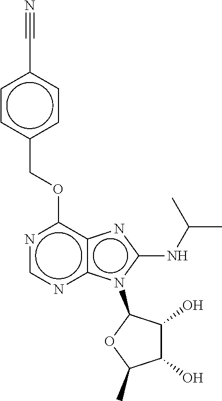 | 6 | propan-2-amine | 4- (hydroxymethyl)benzonitrile | 4 | 0.448 | 425.1 | 425.19 |
| 41 | 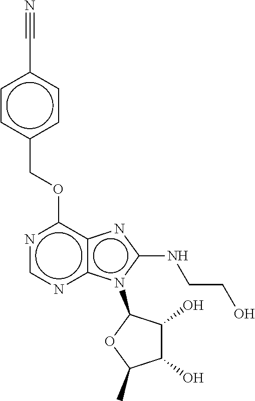 | 6 | 2-aminoethan-1-ol | 4- (hydroxymethyl)benzonitrile | 4 | 0.375 | 427.1 | 427.17 |
| 43 | 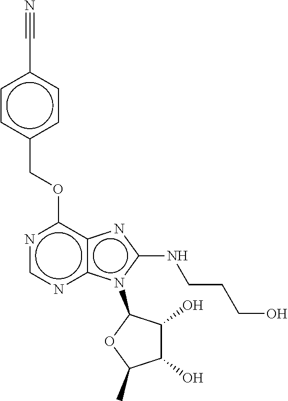 | 6 | 3-aminopropan-1- ol | 4- (hydroxymethyl)benzonitrile | 4 | 0.390 | 441.1 | 441.18 |
| 45 | 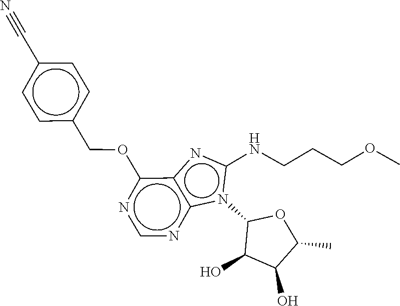 | 6 | 3-methoxypropan-1- amine | 4- (hydroxymethyl)benzonitrile | 4 | 0.365 | 455.1 | 455.20 |
| 47 | 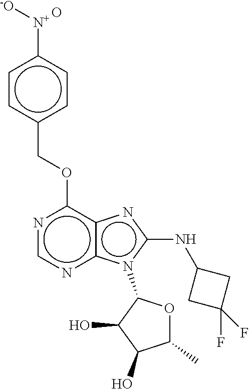 | 6 | 3,3- difluorocyclobutan- 1-amine | (4-nitrophenyl)methanol | 4 | 0.443 | 493.2 | 493.16 |
| 48 | 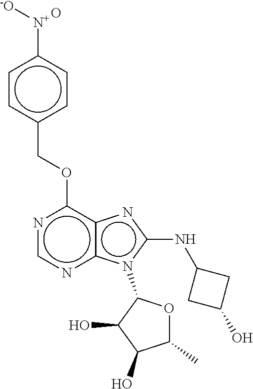 | 6 | (1s,3s)-3- aminocyclobutan-1- ol | (4-nitrophenyl)methanol | 4 | 0.362 | 473.2 | 473.17 |
| 49 | 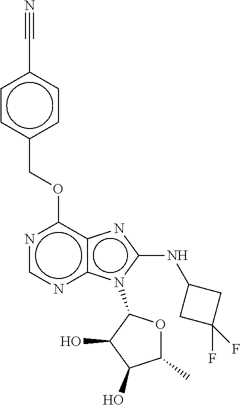 | 6 | 3,3- difluorocyclobutan- 1-amine | 4- (hydroxymethyl)benzonnitrile | 4 | 0.427 | 473.1 | 473.17 |
| 50 | 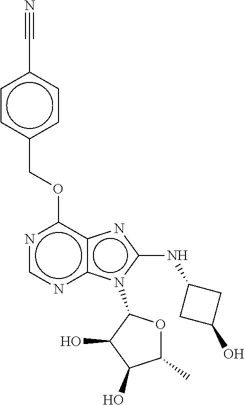 | 6 | (1r,3r)-3- aminocyclobutan-1- ol | 4- (hydroxymethyl)benzonitrile | 4 | 0.434 | 453.3 | 453.18 |
| 51 | 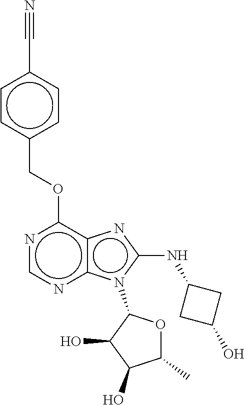 | 6 | (1s,3s)-3- aminocyclobutan-1- ol | 4- (hydroxymethyl)benzonitrile | 4 | 0.346 | 453.2 | 453.18 |
| 52 | 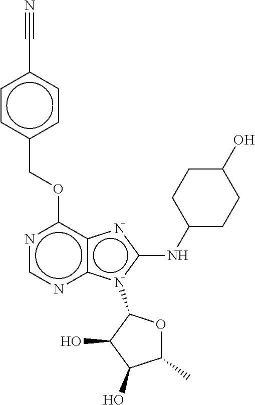 | 6 | 4-aminocyclohexan- 1-ol | 4- (hydroxymethyl)benzonitrile | 4 | 0.411 | 481.2 | 481.21 |
| 54 | 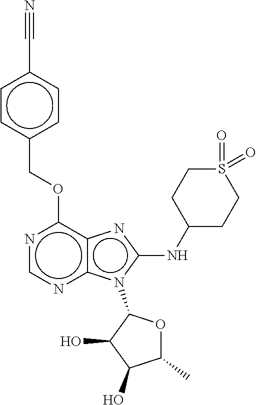 | 6 | 4-aminotetrahydro- 2H-thiopyran 1,1- dioxide | 4- (hydroxymethyl)benzonitrile | 4 | 0.426 | 515.1 | 515.16 |
| 55 | 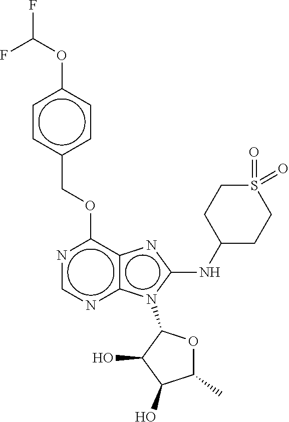 | 10 | 4-aminotetrahydro- 2H-thiopyran 1,1- dioxide | (4- (difluoromethoxy)benzyl)meth- anol | 4 | 0.408 | 556.2 | 556.16 |
| 56 | 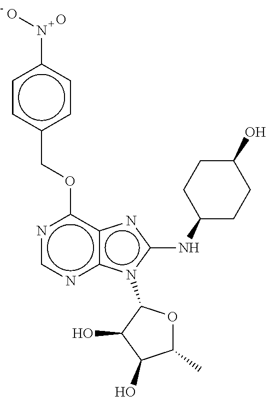 | 6 | (1s,4s)-4- aminocyclohexan-1- ol | (4-nitrophenyl)methanol | 4 | 0.381 | 501.2 | 501.20 |
| 57 | 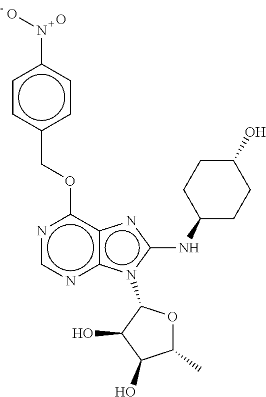 | 6 | (1r,4r)-4- aminocyclohexan-1- ol | (4-nitrophenyl)methanol | 4 | 0.422 | 501.1 | 501.20 |
| 58 | 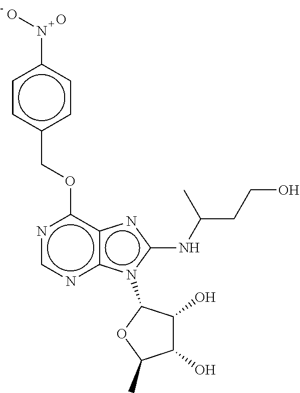 | 6 | 3-aminobutan-1-ol | (4-nitrophenyl)methanol | 4 | 0.369 | 475.2 | 475.19 |
| 59 | 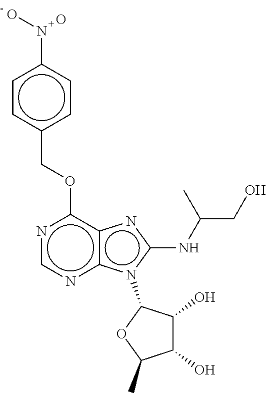 | 6 | 2-aminopropan-1- ol | (4-nitrophenyl)methanol | 4 | 0.363 | 461.1 | 461.17 |
| 60 | 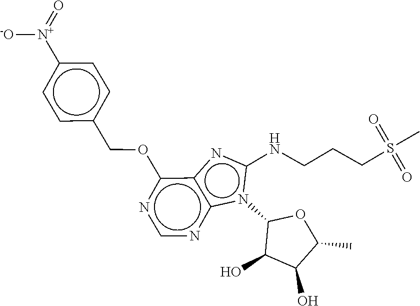 | 10 | 3- (methylsulfonyl)pro- pan-1-amine | (4-nitrophenyl)methanol | 4 | 0.372 | 523.1 | 523.15 |
| 61 | 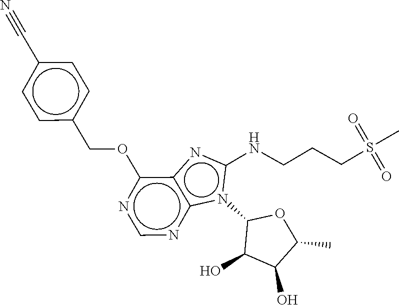 | 10 | 3- (methylsulfonyl)pro- pan-1-amine | 4- (hydroxymethyl)benzonitrile | 4 | 0.308 | 503.3 | 503.16 |
| 62 | 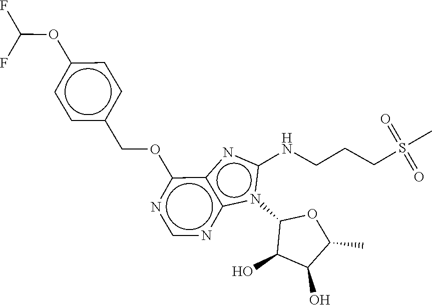 | 10 | 3- (methylsulfonyl)pro- pan-1-amine | (4- (difluoromethoxy)phenyl)meth- anol | 4 | 0.384 | 544 | 544.16 |
| 63 | 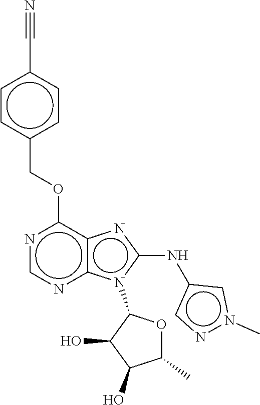 | 10 | 1-methyl-1H- pyrazol-4-amine | 4- (hydroxymethyl)benzonitrile | 4 | 0.464 | 463.3 | 463.18 |
| 64 | 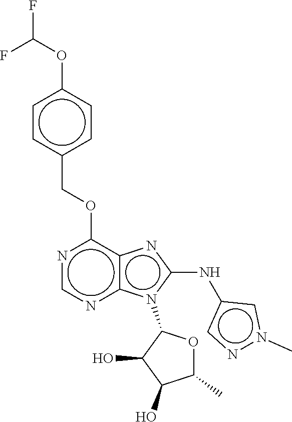 | 10 | 1-methyl-1H- pyrazol-4-amine | (4- (difluoromethoxy)phenyl)meth- anol | 4 | 0.417 | 504.2 | 504.17 |
| 65 | 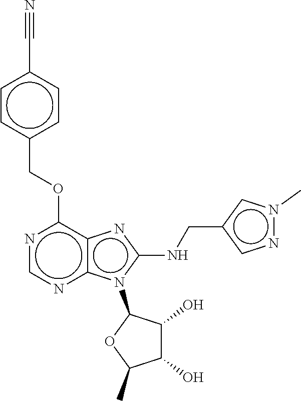 | 10 | (1-methyl-1H- pyrazol-4- yl)methanamine | 4- (hydroxymethyl)benzonitrile | 4 | 0.360 | 477.2 | 477.19 |
| 66 | 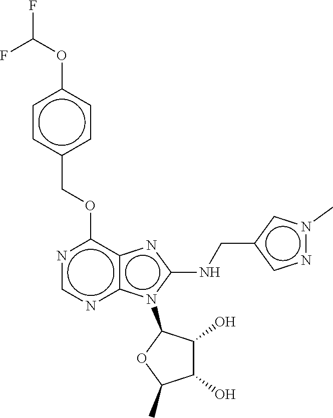 | 10 | (1-methyl-1H- pyrazol-4- yl)methanamine | (4- (difluoromethoxy)phenyl)meth- anol | 4 | 0.375 | 518.3 | 518.19 |
| 67 | 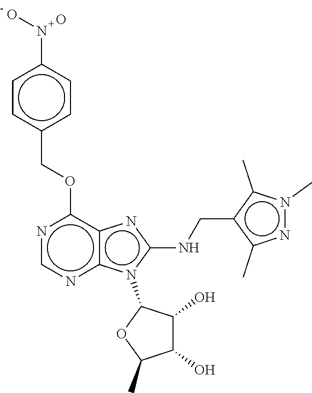 | 10 | (1,3,5-trimethyl-1H- pyrazol-4- yl)methanamine | (4-nitrophenyl)methanol | 4 | 0.488 | 525.1 | 525.21 |
| 68 | 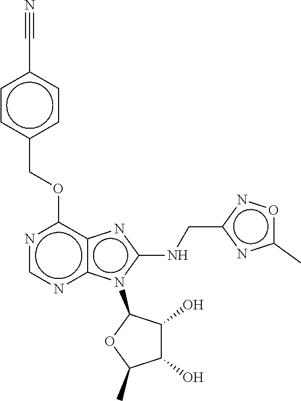 | 10 | (5-methyl-1,2,4- oxadiazol-3- yl)methanamine | 4- (hydroxymethyl)benzonitrile | 4 | 0.469 | 479.1 | 479.17 |
| 69 | 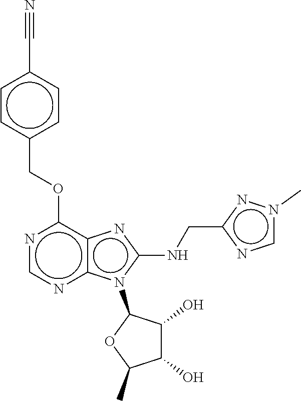 | 10 | (1-methyl-1H-1,2,4- triazol-3- yl)methanamine | 4- (hydroxymethyl)benzonitrile | 4 | 0.339 | 478.2 | 478.19 |
| 70 | 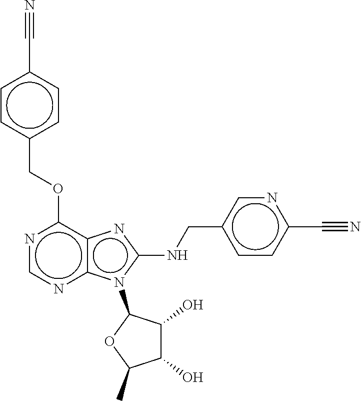 | 10 | 5- (aminomethyl)picoli- nonitrile | 4- (hydroxymethyl)benzonitrile | 4 | 0.491 | 499.1 | 499.18 |
| 71 | 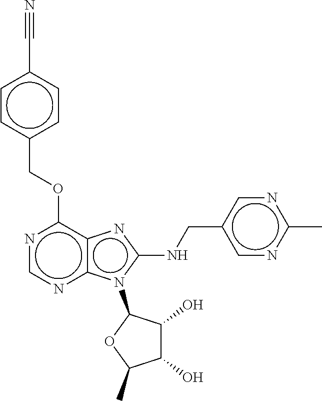 | 10 | (2-methylpyrimidin- 5-yl)methanamine | 4- (hydroxymethyl)benzonitrile | 4 | 0.444 | 489.2 | 489.19 |
| 72 | 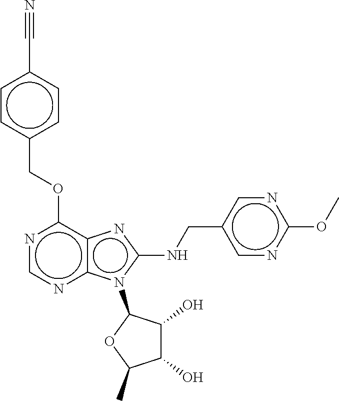 | 10 | (2- methoxypyrimidin- 5-yl)methanamine | 4- (hydroxymethyl)benzonitrile | 4 | 0.376 | 505.2 | 505.19 |
| 73 | 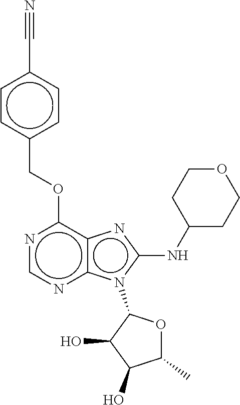 | 10 | tetrahydro-2H- pyran-4-amine | 4- (hydroxymethyl)benzonitrile | 4 | 0.415 | 467.2 | 467.20 |
| 74 | 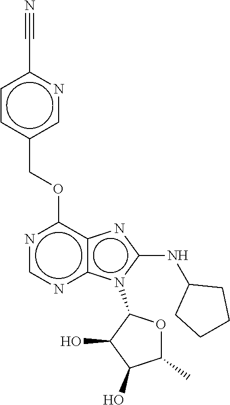 | 10 | cyclopentanamine | 5- (hydroxymethyl)picolinonitrile | 4 | 0.407 | 452.2 | 452.20 |
| 76 | 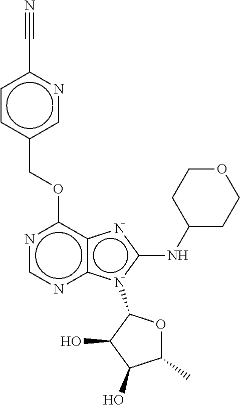 | 10 | tetrahydro-2H- pyran-4-amine | 5- (hydroxymethyl)picolinonitrile | 4 | 0.375 | 468.2 | 468.19 |
| 77 | 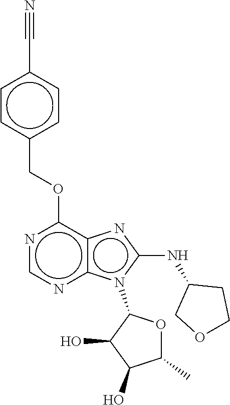 | 10 | (R)-tetrahydrofuran- 3-amine | 4- (hydroxymethyl)benzonitrile | 4 | 0.325 | 453.3 | 453.18 |
| 78 | 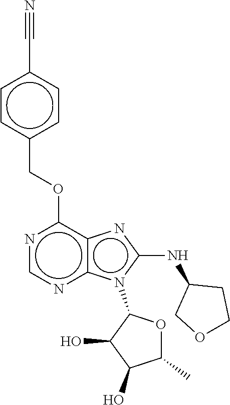 | 10 | (S)-tetrahydrofuran- 3-amine | 4- (hydroxymethyl)benzonitrile | 4 | 0.409 | 453.2 | 453.18 |
| 79 | 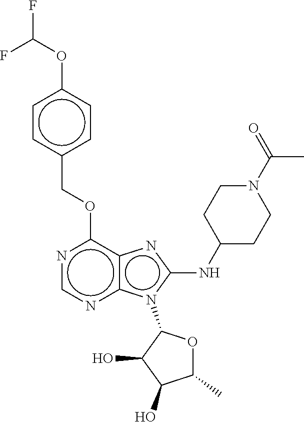 | 10 | 1-(4- aminopiperidin-1- yl)ethan-1-one | (4- (difluoromethoxy)phenyl)meth- anol | 4 | 0.362 | 549.3 | 549.22 |
| 80 | 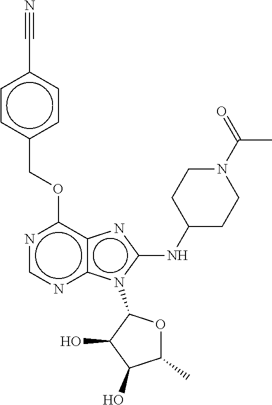 | 10 | 1-(4- aminopiperidin-1- yl)ethan-1-one | 4- (hydroxymethyl)benzonitrile | 4 | 0.356 | 508.1 | 508.22 |
| 81 | 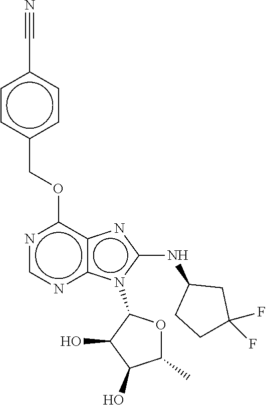 | 10 | (R)-3,3- difluorocyclopentan- 1-amine | 4- (hydroxymethyl)benzonitrile | 4 | 0.431 | 487.2 | 487.18 |
| 82 | 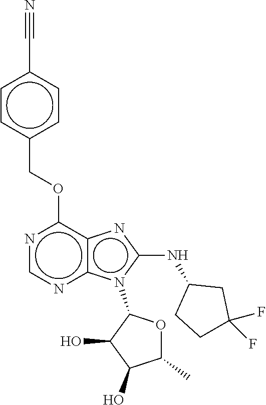 | 10 | (S)-3,3- difluorocyclopentan- 1-amine | 4- (hydroxymethyl)benzonitrile | 4 | 0.419 | 487.2 | 487.18 |
| 83 | 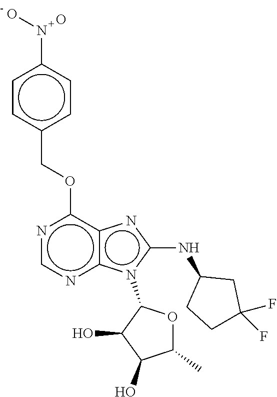 | 10 | (R)-3,3- difluorocyclopentan- 1-amine | (4-nitrophenyl)methanol | 4 | 0.438 | 507.3 | 507.17 |
| 84 | 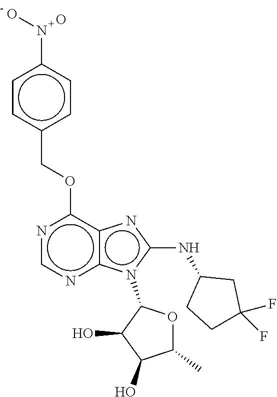 | 10 | (S)-3,3- difluorocyclopentan- 1-amine | (4-nitrophenyl)methanol | 4 | 0.435 | 507.1 | 507.17 |
| 85 | 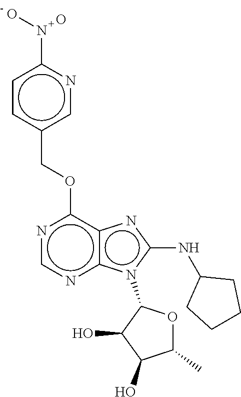 | 10 | cyclopentanamine | (6-nitropyridin-3- yl)methanol | 4 | 0.356 | 472.2 | 472.19 |
| 86 | 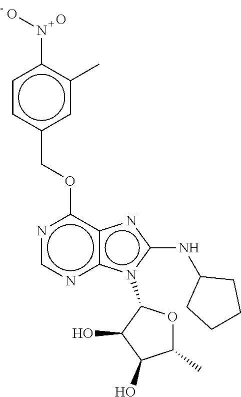 | 10 | cyclopentanamine | (3-methyl-4- nitrophenyl)methanol | 4 | 0.515 | 485.2 | 485.21 |
| 87 | 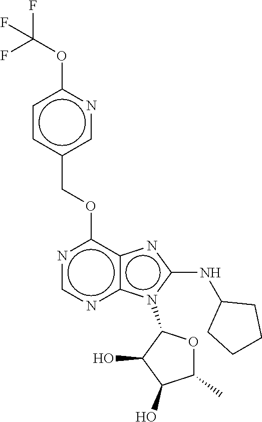 | 10 | cyclopentanamine | (6-(difluoromethoxy)pyridin- 3-yl)methanol | 4 | 0.424 | 511.3 | 511.18 |
| 88 | 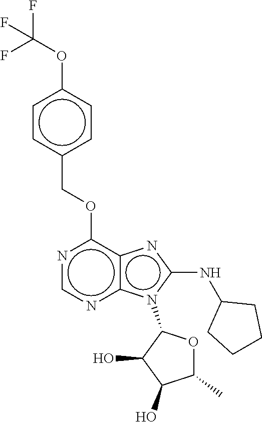 | 10 | cyclopentanamine | (4- (trifluoromethoxy)phenyl)meth- anol | 4 | 0.452 | 510.3 | 510.19 |
| 89 | 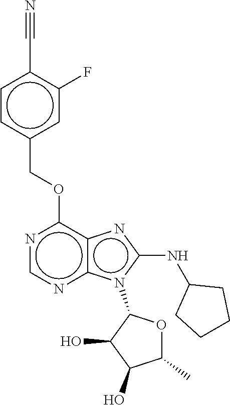 | 10 | cyclopentanamine | 2-fluoro-4- (hydroxymethyl)benzonitrile | 4 | 0.404 | 469.3 | 469.19 |
| 90 | 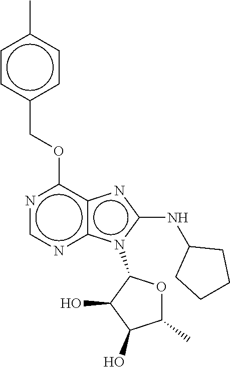 | 10 | cyclopentanamine | p-tolylmethanol | 4 | 0.419 | 440.3 | 440.22 |
| 91 | 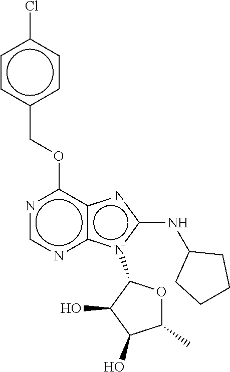 | 10 | cyclopentanamine | (4-chlorophenyl)methanol | 4 | 0.443 | 460.2 | 460.17 |
| 92 | 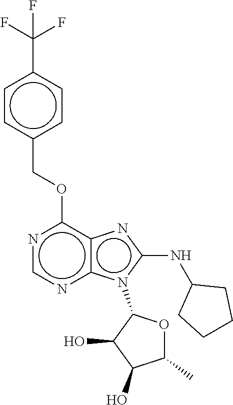 | 10 | cyclopentanamine | (4- (trifluoromethyl)phenyl)meth- anol | 4 | 0.464 | 494.4 | 494.19 |
| 93 | 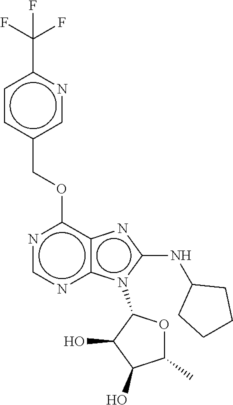 | 10 | cyclopentanamine | (6-(trifluoromethyl)pyridin-3- yl)methanol | 4 | 0.401 | 495.1 | 495.19 |
| 94 | 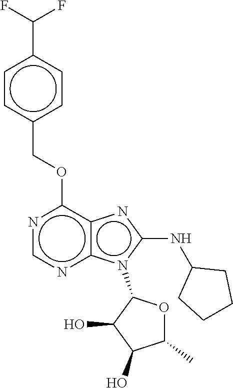 | 10 | cyclopentanamine | (4- (difluoromethyl)phenyl)meth- anol | 4 | 0.420 | 476.3 | 476.20 |
| 95 | 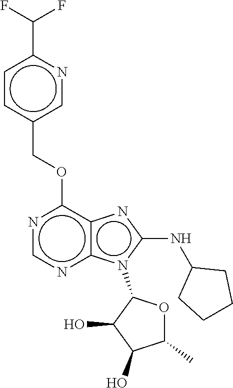 | 10 | cyclopentanamine | (6-(difluoromethyl)pyridin-3- yl)methanol | 4 | 0.375 | 477.1 | 477.20 |
| 96 | 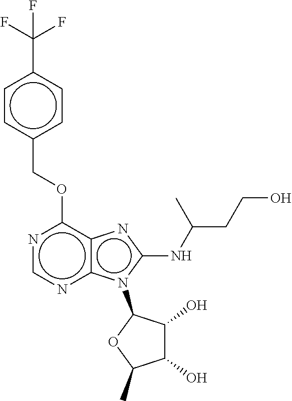 | 10 | 3-aminobutan-1-ol | (4- (trifluoromethyl)phenyl)meth- anol | 4 | 0.403 | 498.3 | 498.19 |
| 97 | 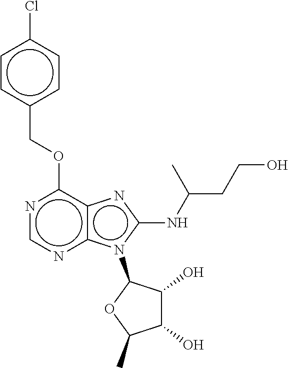 | 10 | 3-aminobutan-1-ol | (4-chlorophenyl)methanol | 4 | 0.391 | 461.1 | 464.16 |
| 98 | 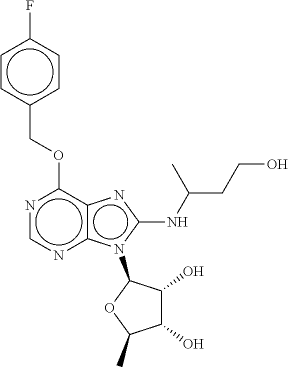 | 10 | 3-aminobutan-1-ol | (4-fluorophenyl)methanol | 4 | 0.366 | 448.1 | 448.19 |
| 99 | 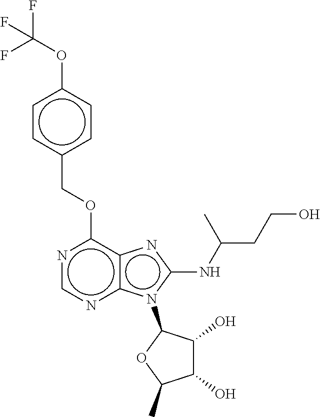 | 10 | 3-aminobutan-1-ol | (4- (trifluoromethoxy)phenyl)meth- anol | 4 | 0.405 | 514.3 | 514.18 |
| 100 | 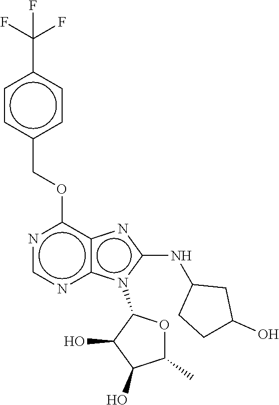 | 10 | 3- aminocyclopentan- 1-ol | (4- (trifluoromethyl)phenyl)meth- anol | 4 | 0.411 | 510.1 | 510.19 |
| 101 | 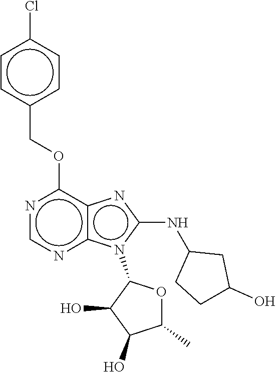 | 10 | 3- aminocyclopentan- 1-ol | (4-chlorophenyl)methanol | 4 | 0.402 | 476.1 | 476.16 |
| 102 | 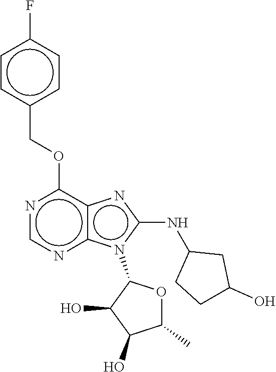 | 10 | 3- aminocyclopentan- 1-ol | (4-fluorophenyl)methanol | 4 | 0.368 | 460.2 | 460.19 |
| 103 | 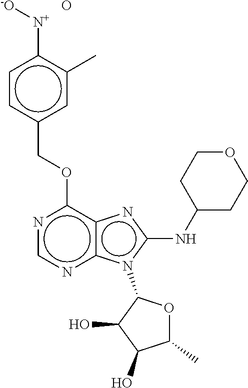 | 10 | tetrahydro-2H- pyran-4-amine | (3-methyl-4- nitrophenyl)methanol | 4 | 0.408 | 501.2 | 501.20 |
| 104 | 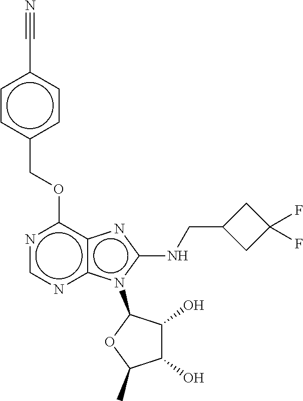 | 10 | 3,3- difluorocyclobutan- 1-amine | 4- (hydroxymethyl)benzonitrile | 4 | 0.419 | 487.2 | 487.18 |
| 105 | 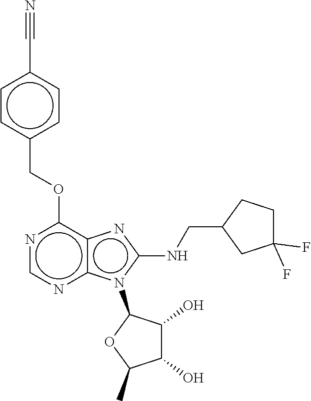 | 10 | (3,3- difluorocyclopentyl) methanamine | 4- (hydroxymethyl)benzonitrile | 4 | 0.421 | 501.2 | 501.20 |
| 106 | 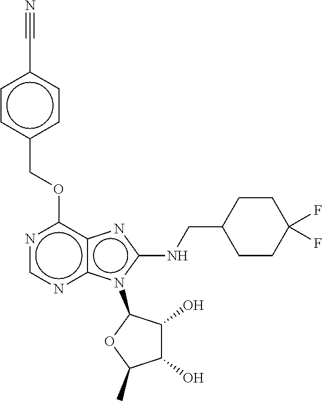 | 10 | (4,4- difluorocyclohexyl) methanamine | 4- (hydroxymethyl)benzonitrile | 4 | 0.428 | 515.3 | 515.21 |
| 107 | 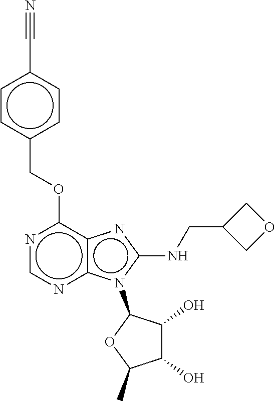 | 10 | oxetan-3- ylmethanamine | 4- (hydroxymethyl)benzonitrile | 4 | 0.315 | 453.1 | 453.18 |
| 108 | 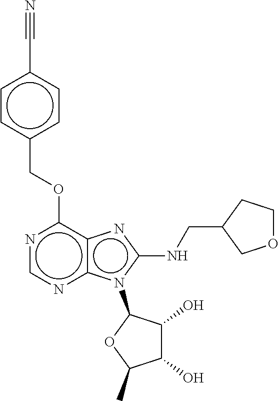 | 10 | (tetrahydrofuran-3- yl)methanamine | 4- (hydroxymethyl)benzonitrile | 4 | 0.357 | 467.2 | 467.20 |
| 109 | 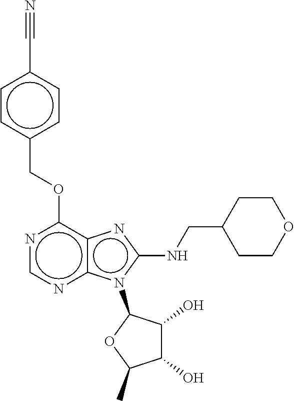 | 10 | (tetrahydro-2H- pyran-4- yl)methanamine | 4- (hydroxymethyl)benzonitrile | 4 | 0.364 | 481.2 | 481.21 |
| 110 | 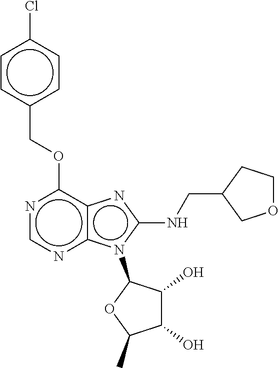 | 10 | (tetrahydrofuran-3- yl)methanamine | (4-chlorophenyl)methanol | 4 | 0.415 | 476.1 | 476.16 |
| 111 | 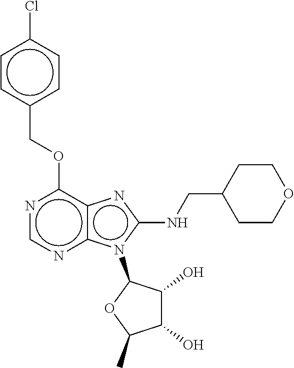 | 10 | (tetrahydro-2H- pyran-4- yl)methanamine | (4-chlorophenyl)methanol | 4 | 0.415 | 490.2 | 490.18 |
| 112 | 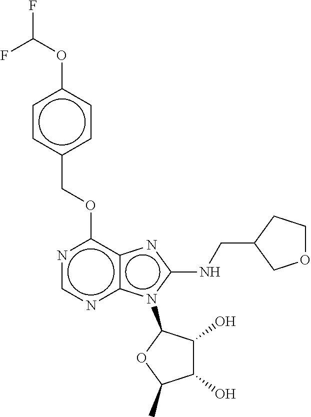 | 10 | (tetrahydrofuran-3- yl)methanamine | (4- (difluoromethoxy)phenyl)meth- anol | 4 | 0.398 | 508.3 | 508.19 |
| 113 | 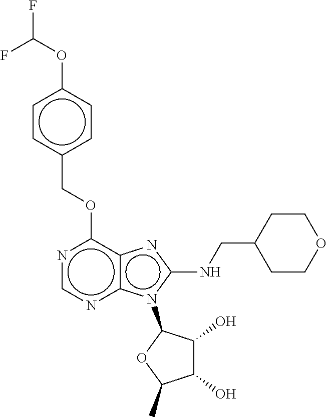 | 10 | (tetrahydro-2H- pyran-4- yl)methanamine | (4- (difluoromethoxy)phenyl)meth- anol | 4 | 0.397 | 522.1 | 522.21 |
| 114 | 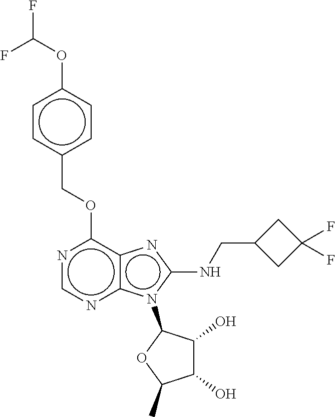 | 10 | (3,3- difluorocyclobutyl) methanamine | (6-(difluoromethoxy)pyridin- 3-yl)methanol | 4 | 0.441 | 529.2 | 529.17 |
| 115 | 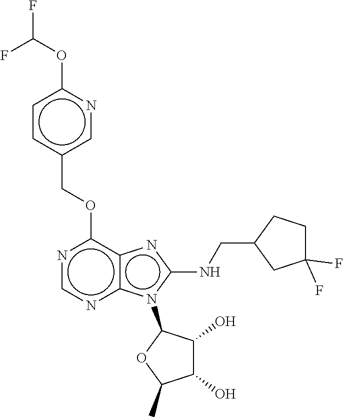 | 10 | (3,3- difluorocyclopentyl) methanamine | (6-(difluoromethoxy)pyridin- 3-yl)methanol | 4 | 0.434 | 543.2 | 543.19 |
| 116 | 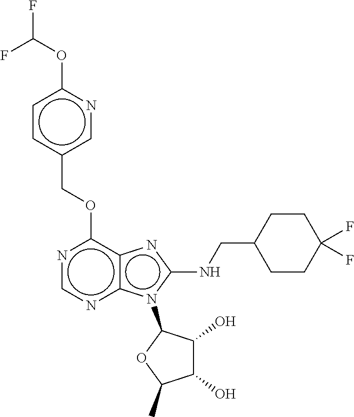 | 10 | (4,4- difluorocyclohexyl) methanamine | (6-(difluoromethoxy)pyridin- 3-yl)methanol | 4 | 0.441 | 557.3 | 557.21 |
| 117 | 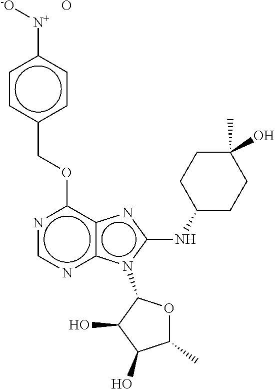 | 10 | 4-amino-1- methylcyclohexan- 1-ol | (4-nitrophenyl)methanol | 4 | 0.379 | 515.3 | 515.22 |
| 118 | 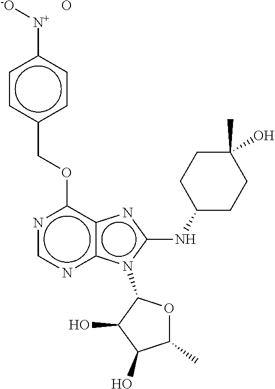 | 10 | 4-amino-1- methylcyclohexan- 1-ol | (4-nitrophenyl)methanol | 4 | 0.337 | 515.3 | 515.22 |
| 119 | 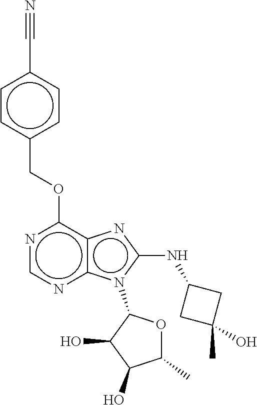 | 10 | 3-amino-1- methylcyclobutan-1- ol | 4- (hydroxymethyl)benzonitrile | 4 | 0.345 | 467.1 | 467.20 |
| 120 | 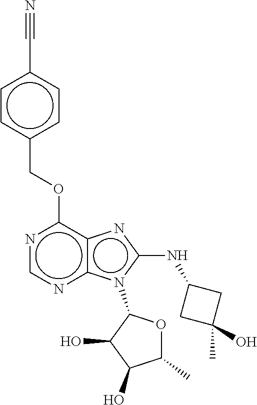 | 10 | 3-amino-1- methylcyclobutan-1- ol | 4- (hydroxymethyl)benzonitrile | 4 | 0.342 | 467.3 | 467.20 |
| 121 | 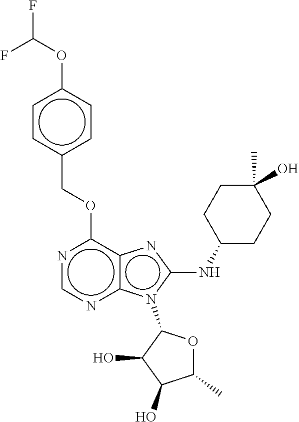 | 10 | 4-amino-1- methylcyclohexan- 1-ol | (4- (difluoromethoxy)phenyl)meth- anol | 4 | 0.395 | 536.3 | 536.22 |
| 122 | 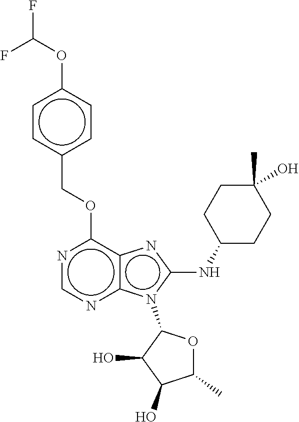 | 10 | 4-amino-1- methylcyclohexan- 1-ol | (4- (difluoromethoxy)phenyl)meth- anol | 4 | 0.448 | 536.4 | 536.22 |
| 123 | 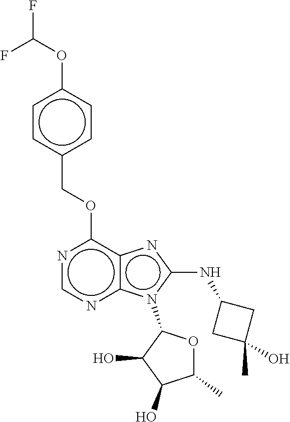 | 10 | 3-amino-1- methylcyclobutan-1- ol | (4- (difluoromethoxy)phenyl)meth- anol | 4 | 0.385 | 508.1 | 508.19 |
| 124 | 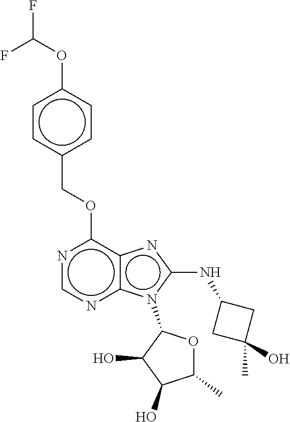 | 10 | 3-amino-1- methylcyclobutan-1- ol | (4- (difluoromethoxy)phenyl)meth- anol | 4 | 0.381 | 508.2 | 508.19 |
| 125 | 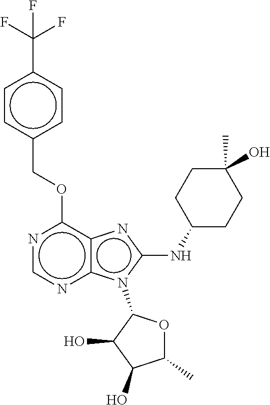 | 10 | 4-amino-1- methylcyclohexan- 1-ol | (4- (trifluoromethyl)phenyl)meth- anol | 4 | 0.419 | 538.2 | 538.22 |
| 126 | 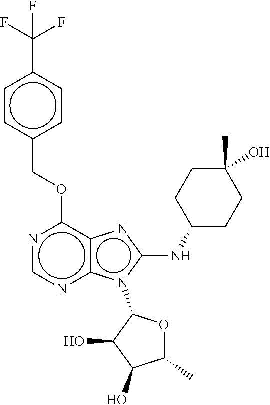 | 10 | 4-amino-1- methylcyclohexan- 1-ol | (4- (trifluoromethyl)phenyl)meth- anol | 4 | 0.418 | 538.2 | 538.22 |
| 127 | 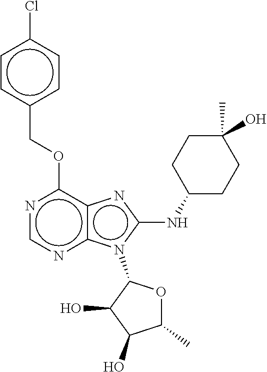 | 10 | 4-amino-1- methylcyclohexan- 1-ol | (4-chlorophenyl)methanol | 4 | 0.404 | 504.1 | 504.19 |
| 128 | 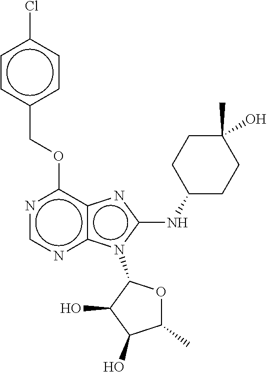 | 10 | 4-amino-1- methylcyclohexan- 1-ol | (4-chlorophenyl)methanol | 4 | 0.404 | 504.1 | 504.19 |
| 129 | 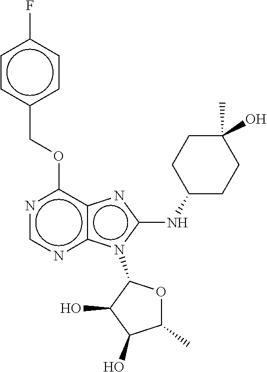 | 10 | 4-amino-1- methylcyclohexan- 1-ol | (4-fluorophenyl)methanol | 4 | 0.382 | 488.2 | 488.22 |
| 130 | 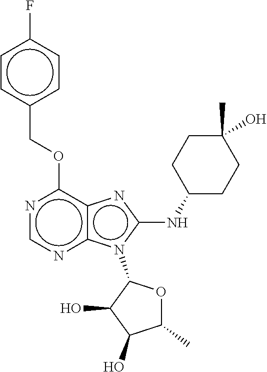 | 10 | 4-amino-1- methylcyclohexan- 1-ol | (4-fluorophenyl)methanol | 4 | 0.446 | 488.3 | 488.22 |
| 131 | 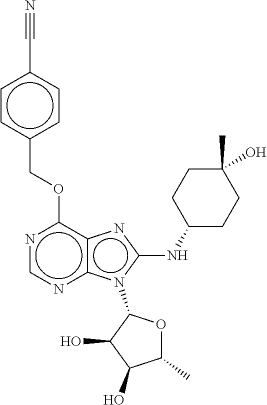 | 10 | 4-amino-1- methylcyclohexan- 1-ol | 4- (hydroxymethyl)benzonitrile | 4 | 0.355 | 495.3 | 495.23 |
| 132 | 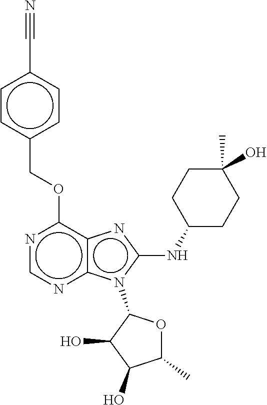 | 10 | 4-amino-1- methylcyclohexan- 1-ol | 4- (hydroxymethyl)benzonitrile | 4 | 0.365 | 495.2 | 495.23 |
| 133 | 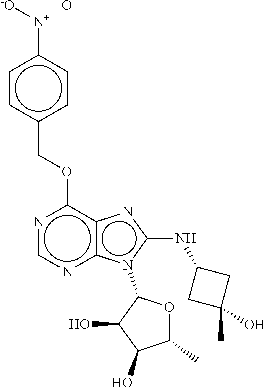 | 10 | 3-amino-1- methylcyclobutan-1- ol | (4-nitrophenyl)methanol | 4 | 0.369 | 487.1 | 487.19 |
| 134 | 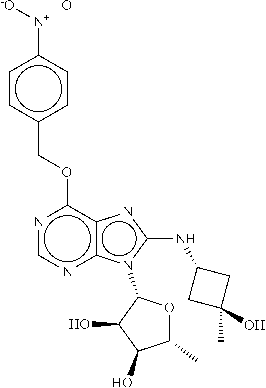 | 10 | 3-amino-1- methylcyclobutan-1- ol | (4-nitrophenyl)methanol | 4 | 0.359 | 487.2 | 487.19 |
| 423 | 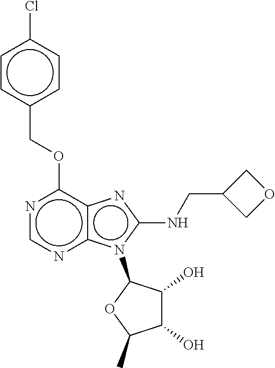 | 10 | oxetan-3- ylmethanamine | (4-chlorophenyl)methanol | 4 | 0.347 | 462.2 | 462.15 |
| 424 | 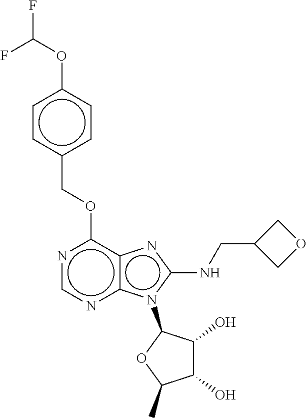 | 10 | oxetan-3- ylmethanamine | (4- (difluoromethoxy)phenyl)meth- anol | 4 | 0.354 | 494.2 | 494.18 |

TABLE 3

| | | Inter- | | Gen- | | | Calcu- |
|---|---|---|---|---|---|---|---|
| | | mediate | | eral | LCMS | LCMS | lated |
| Comp. | | com- | | pro- | Rt | (M + | (M + |
| No. | Structure | pound | Amine | cedure | (min) | H)+ | H)+ |
| 4 | 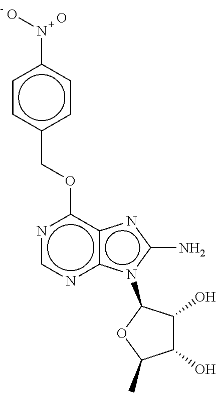 | 3 | ammonia | 3 | 0.380 | 403.2 | 403.13 |
| 5 | 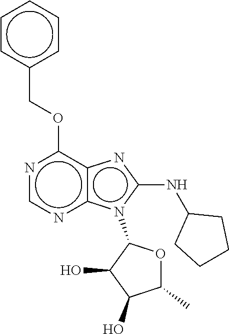 | 4 | cyclo- pentanamine | 3 | 0.499 | 426.2 | 426.21 |
| 6 | 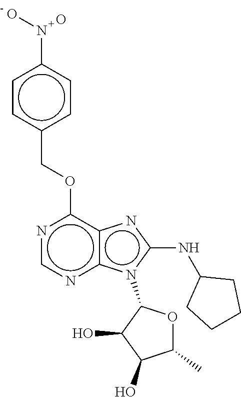 | 3 | cyclo- pentanamine | 3 | 0.475 | 471.3 | 471.19 |
| 7 | 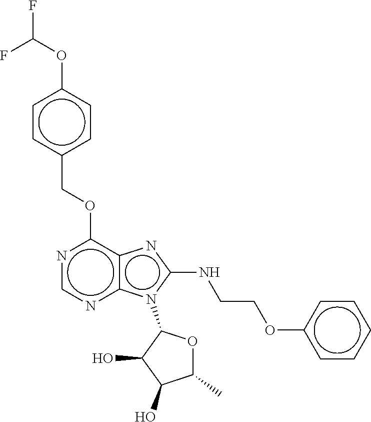 | 5 | 2-phenoxy- ethan-1- amine | 3 | 0.542 | 544.2 | 544.19 |
| 8 | 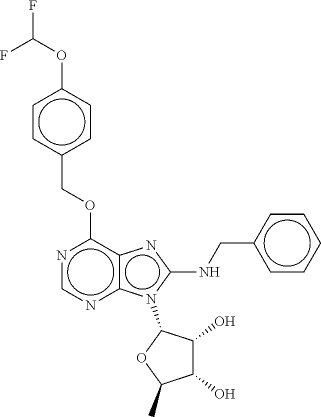 | 5 | phenyl- methanamine | 3 | 0.429 | 514.2 | 514.18 |
| 9 | 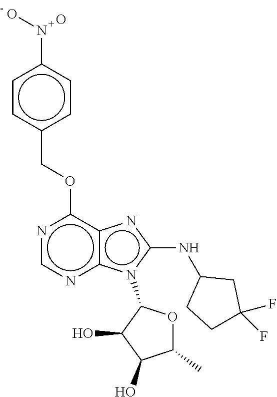 | 3 | 3,3-difluoro- cyclopentan- 1-amine | 3 | 0.506 | 507.2 | 507.17 |
| 10 | 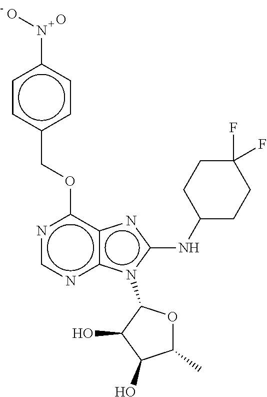 | 3 | 4,4-difluoro- cyclohexan- 1-amine | 3 | 0.460 | 521.2 | 521.19 |
| 11 | 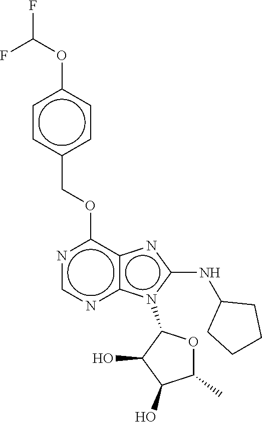 | 3 | cyclo- pentanamine | 3 | 0.534 | 492.2 | 492.20 |
| 16 | 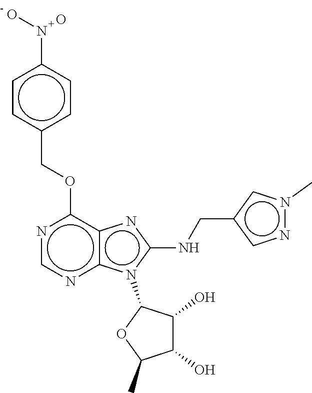 | 3 | (1-methyl-1H- pyrazol-4- yl)methanamine | 3 | 0.379 | 497.2 | 497.18 |
| 17 | 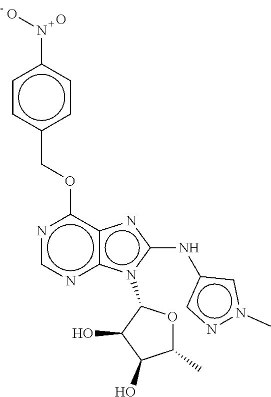 | 3 | 1-methyl-1H- pyrazol- 4-amine | 3 | 0.451 | 483.3 | 483.17 |
| 18 | 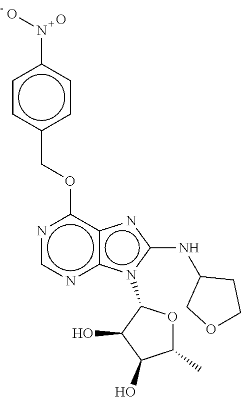 | 3 | tetrahydrofuran-3- amine | 3 | 0.443 | 473.0 | 473.17 |
| 19 | 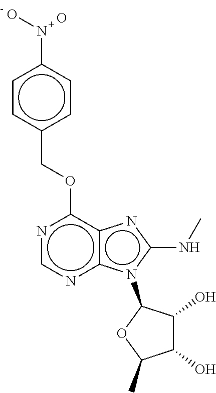 | 3 | methanamine | 3 | 0.354 | 417.1 | 417.14 |
| 20 | 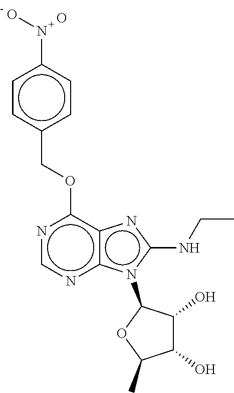 | 3 | ethanamine | 3 | 0.328 | 431.2 | 431.16 |
| 21 | 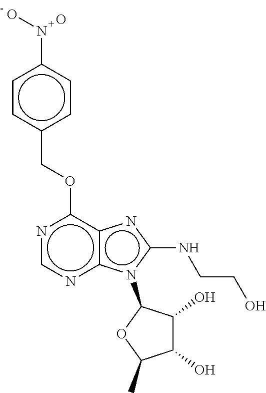 | 3 | 2-aminoethan- 1-ol | 3 | 0.399 | 447.2 | 447.15 |
| 22 | 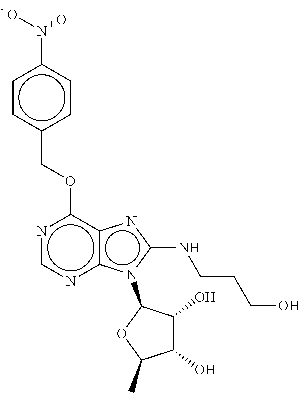 | 3 | 3-amino- propan-1-ol | 3 | 0.404 | 461.1 | 461.17 |
| 23 | 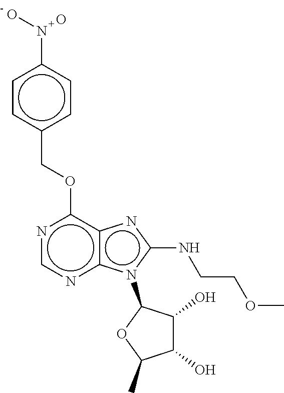 | 3 | 3-methoxy- propan-1- amine | 3 | 0.452 | 461.1 | 461.17 |
| 25 | 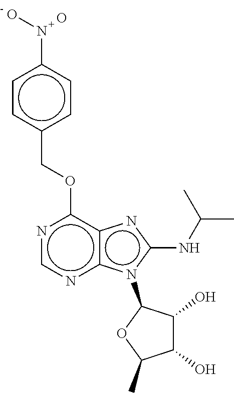 | 3 | propan-2-amine | 3 | 0.444 | 445.1 | 445.18 |
| 26 | 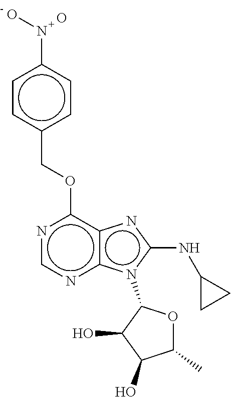 | 3 | cyclopro- panamine | 3 | 0.405 | 443.2 | 443.16 |
| 27 | 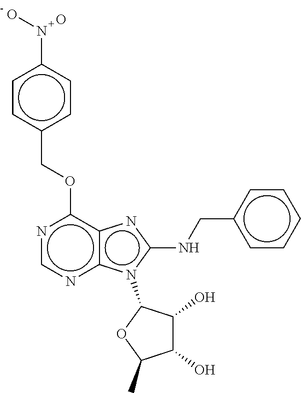 | 3 | phenyl- methanamine | 3 | 0.514 | 493.1 | 493.18 |
| 28 | 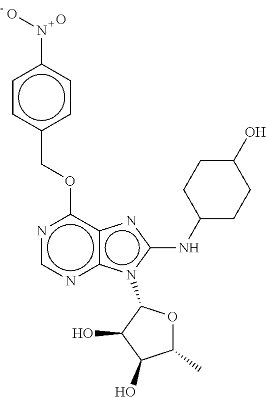 | 3 | 4-aminocyclo- hexan-1-ol | 3 | 0.426 | 501.4 | 501.20 |
| 29 | 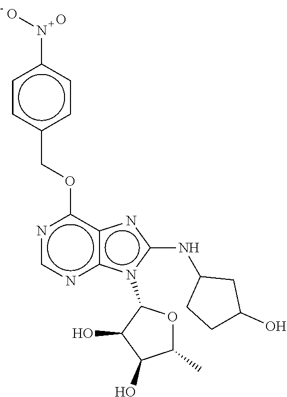 | 3 | 3-aminocyclo- pentan-1-ol | 3 | 0.424 | 487.5 | 487.19 |
| 30 | 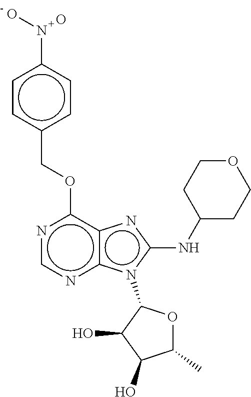 | 3 | tetrahydro-2H- pyran-4-amine | 3 | 0.454 | 487.1 | 487.19 |
| 31 | 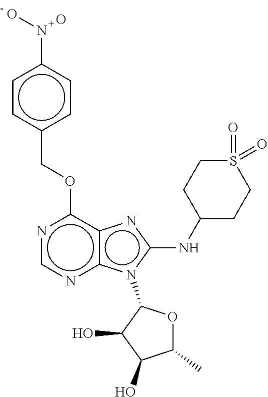 | 3 | 4-aminotetra- hydro-2H- thiopyran 1,1-dioxide | 3 | 0.437 | 535.2 | 535.15 |
| 32 | 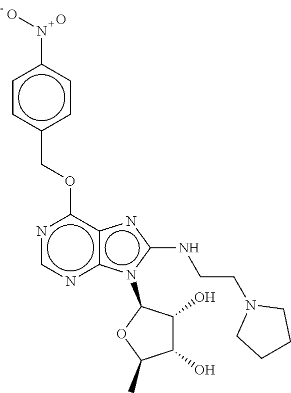 | 3 | 2-(pyrrolidin- 1-yl)ethan- 1-amine | 3 | 0.391 | 500.3 | 500.22 |
| 33 | 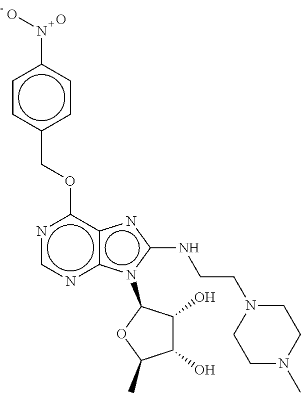 | 3 | 2-(4-methyl- piperazin- 1-yl)ethan-1-amine | 3 | 0.344 | 529.6 | 529.24 |
| 34 | 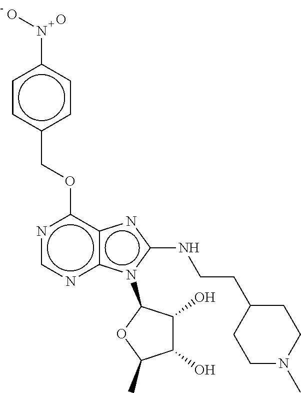 | 3 | 2-(1-methyl- piperidin- 4-yl)ethan-1-amine | 3 | 0.382 | 528.4 | 528.25 |
| 35 | 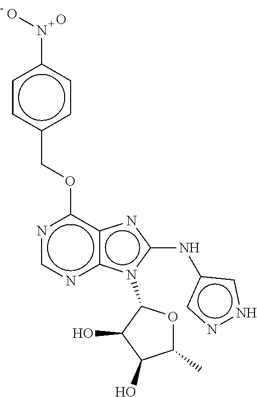 | 3 | 1H-pyrazol- 4-amine | 3 | 0.432 | 469.1 | 469.15 |
| 36 | 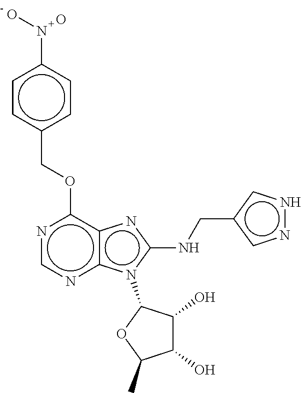 | 3 | (1H-pyrazol-4- yl)methanamine | 3 | 0.419 | 483.0 | 483.17 |
| 38 | 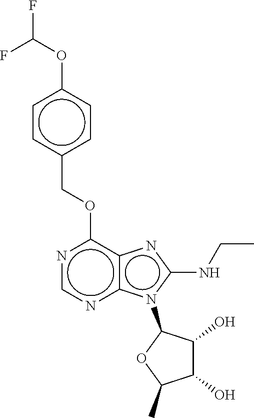 | 9 | ethanamine | 3 | 0.472 | 452.1 | 452.17 |
| 42 | 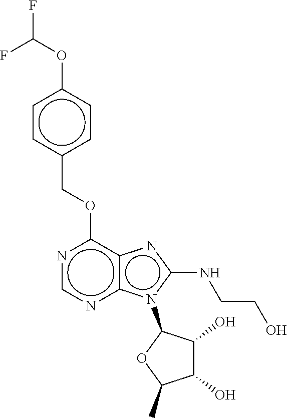 | 9 | 2-amino- ethan-1-ol | 3 | 0.369 | 468.1 | 468.16 |
| 44 | 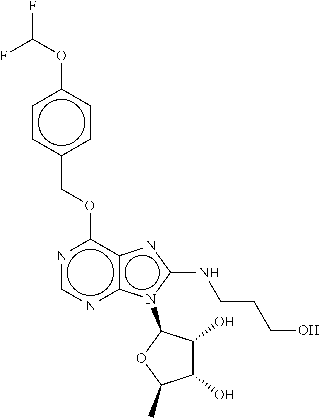 | 9 | 3-amino- propan-1-ol | 3 | 0.429 | 482.2 | 482.18 |
| 46 | 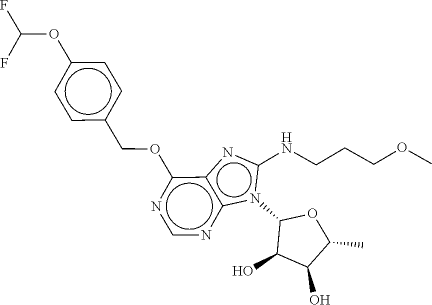 | 9 | 3-methoxy- propan-1- amine | 3 | 0.458 | 496.4 | 496.19 |
| 53 | 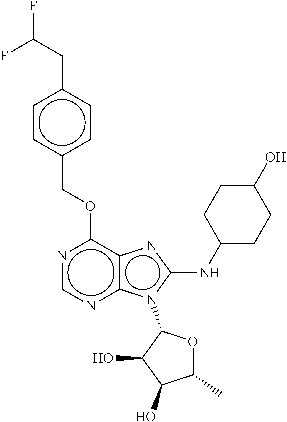 | 9 | 4-aminocyclo- hexan-1-ol | 3 | 0.387 | 522.2 | 522.21 |
| 136 | 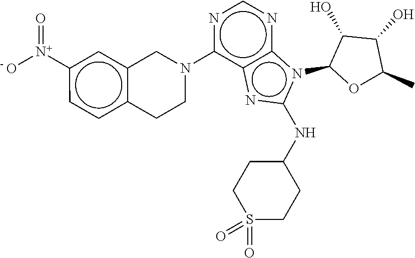 | 18 | 4-aminotetrahydro- 2H-thiopyran 1,1- dioxide | 3 | 0.375 | 560.2 | 560.18 |
| 137 | 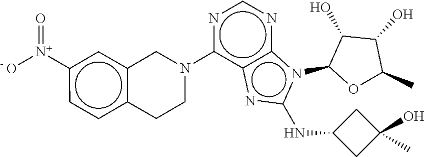 | 19 | 3-amino-1- methylcyclobutan-1- ol | 3 | 0.364 | 512.3 | 512.22 |
| 138 | 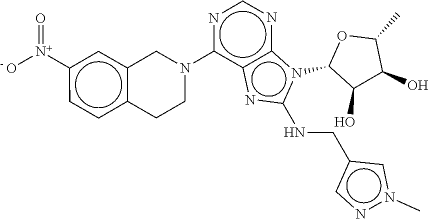 | 19 | (1-methyl-1H- pyrazol- 4-yl)methanamine | 3 | 0.382 | 522.1 | 522.21 |
| 139 | 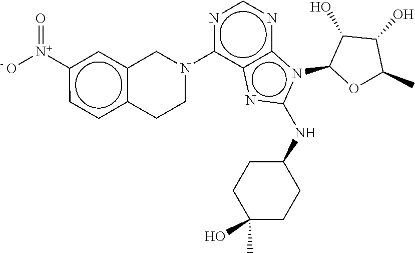 | 19 | Cis-4-amino-1- methylcyclo- hexan-1-ol | 3 | 0.386 | 540.2 | 540.25 |
| 140 | 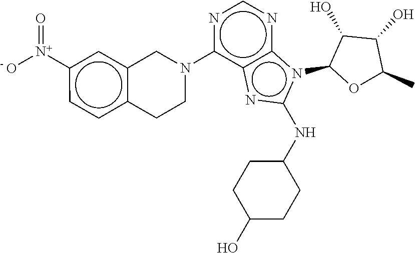 | 18 | 4-aminocyclo- hexan-1-ol | 3 | 0.421 | 526.4 | 526.23 |
| 141 | 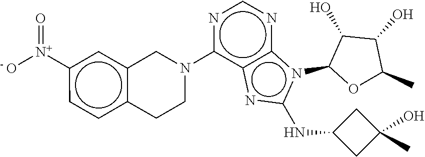 | 19 | 3-amino-1-methyl- cyclobutan-1- ol | 3 | 0.364 | 512.3 | 512.22 |
| 142 | 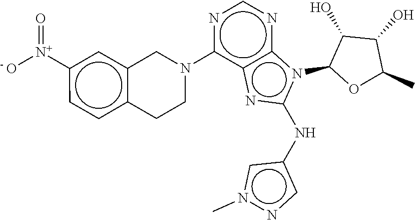 | 19 | 1-methyl-1H- pyrazol-4-amine | 3 | 0.394 | 508.2 | 508.20 |
| 143 | 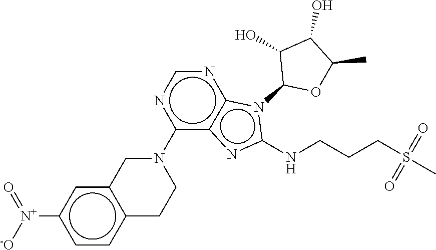 | 19 | 3-(methyl- sulfonyl)pro- pan-1-amine | 3 | 0.364 | 548.2 | 548.18 |
| 144 | 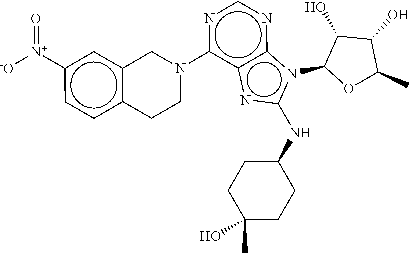 | 19 | Trans-4-amino-1- methylcyclo- hexan-1- ol | 3 | 0.408 | 540.4 | 540.25 |
| 145 | 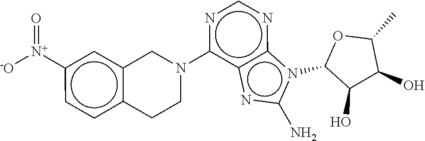 | 19 | ammonia | 3 | 0.397 | 428.1 | 428.16 |
| 146 | 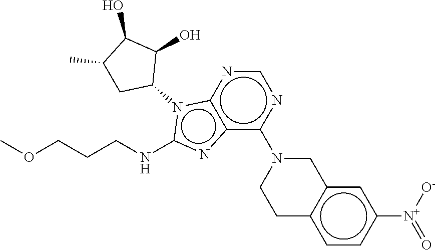 | 19 | 3-methoxy- propan-1- amine | 3 | 0.370 | 500.3 | 500.22 |
| 148 | 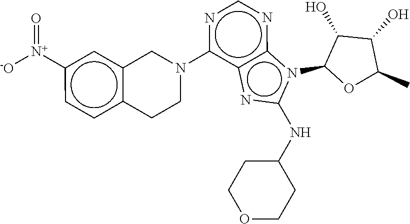 | 19 | tetrahydro-2H- pyran-4-amine | 3 | 0.399 | 512.2 | 512.22 |
| 149 | 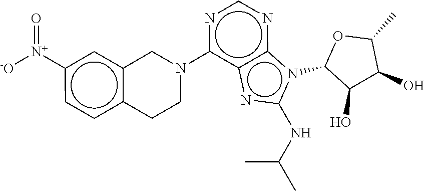 | 19 | propan-2-amine | 3 | 0.391 | 470.3 | 470.21 |
| 150 | 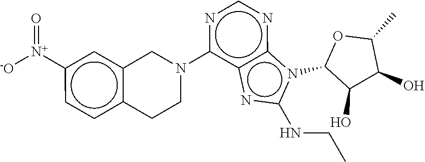 | 18 | ethanamine | 3 | 0.464 | 456.2 | 456.19 |
| 153 | 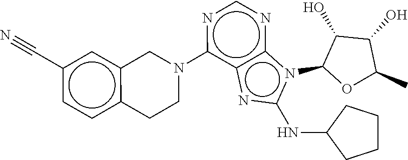 | 20 | cyclo- pentanamine | 3 | 0.500 | 476.3 | 476.23 |
| 154 | 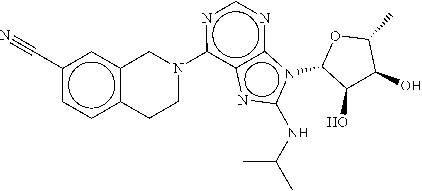 | 20 | propan-2-amine | 3 | 0.411 | 450.3 | 450.22 |
| 155 | 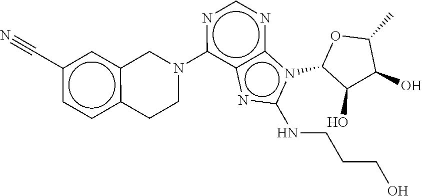 | 20 | 3-aminopropan- 1-ol | 3 | 0.400 | 466.1 | 466.21 |
| 160 | 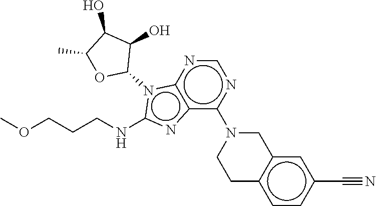 | 20 | 3-methoxypro- pan-1-amine | 3 | 0.453 | 480.2 | 480.23 |
| 164 | 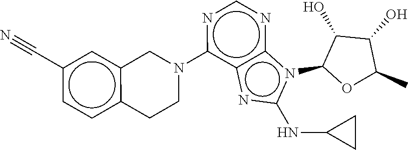 | 19 | cyclopropanamine | 3 | 0.546 | 448.3 | 448.20 |
| 167 | 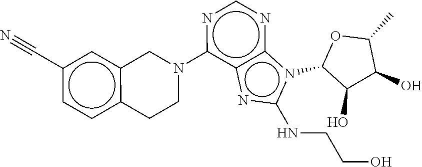 | 20 | 2-aminoethan-1-ol | 3 | 0.386 | 452.1 | 452.20 |
| 170 | 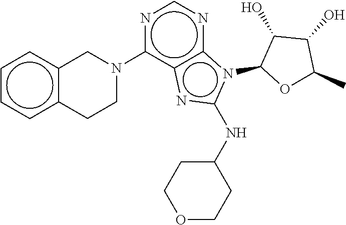 | 21 | tetrahydro-2H- pyran-4-amine | 3 | 0.372 | 467.2 | 467.23 |
| 171 | 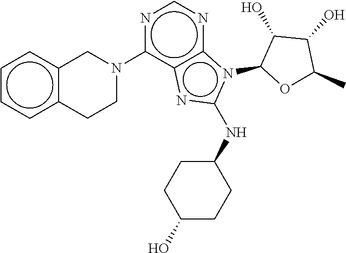 | 21 | trans-4-aminocyclo- hexan-1-ol | 3 | 0.347 | 481.2 | 481.25 |
| 181 | 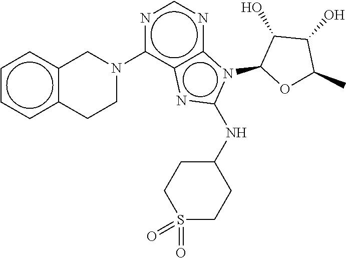 | 21 | 4-aminotetrahydro- 2H-thiopyran 1,1- dioxide | 3 | 0.349 | 515.3 | 515.20 |
| 195 | 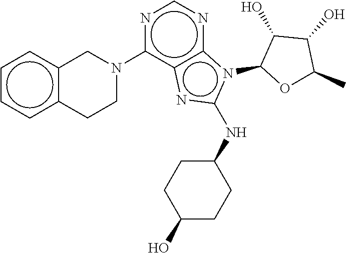 | 21 | cis-4-aminocyclo- hexan-1-ol | 3 | 0.375 | 481.3 | 481.25 |
| 198 | 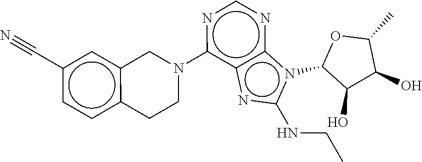 | 20 | ethanamine | 3 | 0.448 | 436.1 | 436.20 |
| 212 | 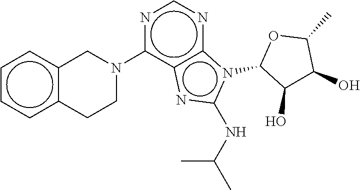 | 20 | propan-2-amine | 3 | 0.402 | 425.1 | 425.22 |
| 264 | 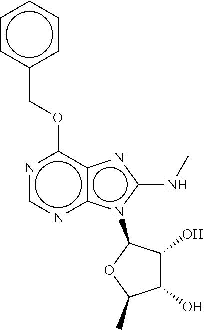 | 4 | methanamine | 3 | 0.307 | 372.1 | 372.16 |
| 355 | 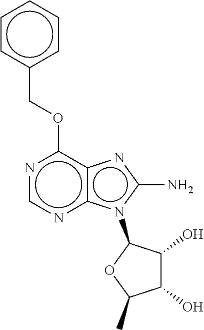 | 4 | ammonia | 3 | 0.410 | 358.1 | 358.14 |

TABLE 4

| Comp. | | Intermediate | | General | LCMS | LCMS | Calculated |
|---|---|---|---|---|---|---|---|
| No. | Structure | compound | Amine | procedure | Rt (min) | (M + H)+ | (M + H)+ |
| 185 | 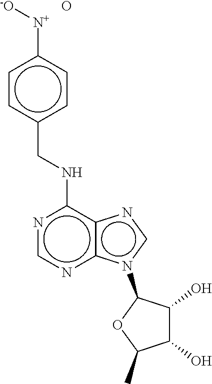 | 1 | (4- nitrophenyl)methan- amine | 7 | 0.764 | 387.0 | 387.13 |
| 196 | 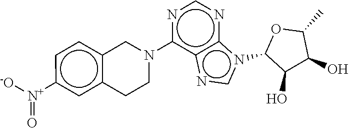 | 1 | 6-nitro-1,2,3,4- tetrahydroisoquino- line | 7 | 0.815 | 412.9 | 413.15 |
| 213 | 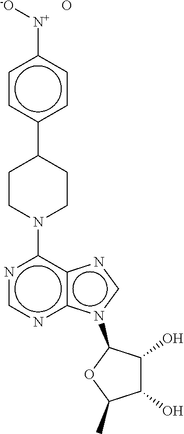 | 1 | 4-(4- nitrophenyl)piperi- dine | 7 | 0.874 | 441.2 | 441.18 |
| 252 | 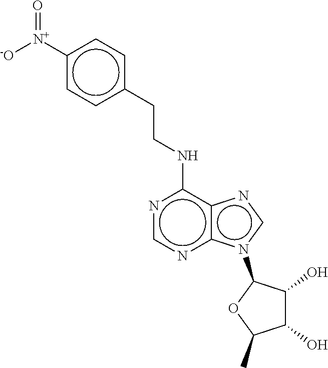 | 1 | 2-(4- nitrophenyl)ethan- 1-amine | 7 | 0.765 | 401.3 | 401.15 |
| 255 | 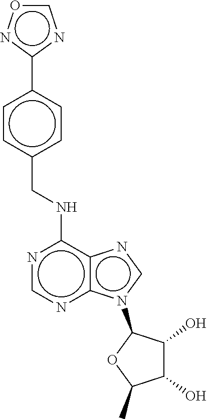 | 1 | (4-(1,2,4- oxadiazol-3- yl)phenyl)methana- mine | 7 | 0.732 | 410.1 | 410.15 |
| 277 | 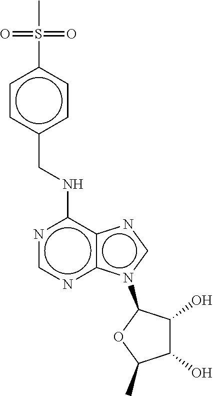 | 1 | (4- (methylsulfonyl)phe- nyl)methanamine | 7 | 0.704 | 420.1 | 420.13 |
| 282 | 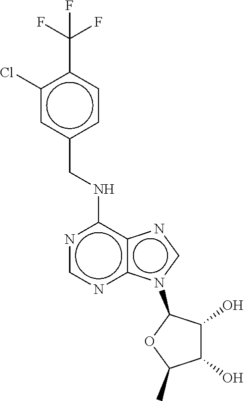 | 1 | (3-chloro-4- (trifluoromethyl)phe- nyl)methanamine | 7 | 0.872 | 444.2 | 444.10 |
| 299 | 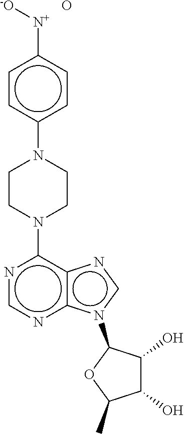 | 1 | 1-(4- nitrophenyl)pipera- zine | 7 | 0.902 | 442.2 | 442.18 |
| 315 | 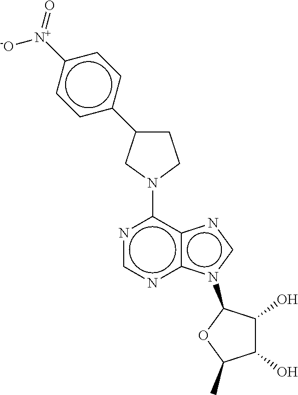 | 1 | 3-(4- nitrophenyl)pyrroli- dine | 7 | 0.773 | 427.0 | 427.17 |
| 320 | 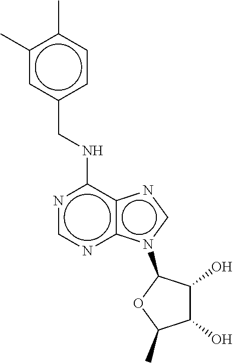 | 1 | (3,4- dimethylphenyl)meth- anamine | 7 | 0.777 | 370.1 | 370.18 |
| 322 | 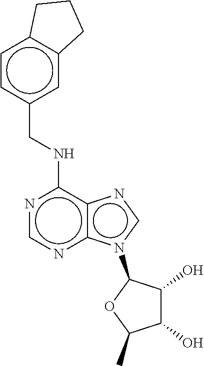 | 1 | (2,3-dihydro-1H- inden-5- yl)methanamine | 7 | 0.809 | 382.0 | 382.18 |
| 324 | 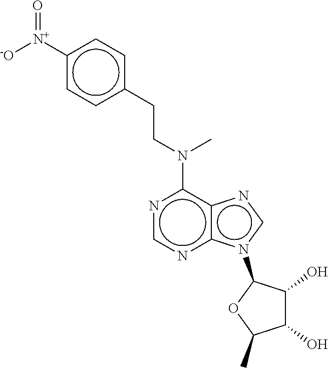 | 1 | N-methyl-2-(4- nitrophenyl)ethan- 1-amine | 7 | 0.807 | 415.1 | 415.17 |
| 327 | 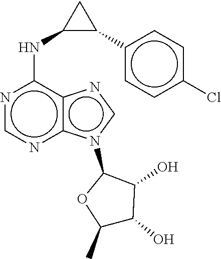 | 1 | (1S,2R)-2-(4- chlorophenyl)cyclo- propan-1-amine | 7 | 0.810 | 402.3 | 402.13 |
| 331 | 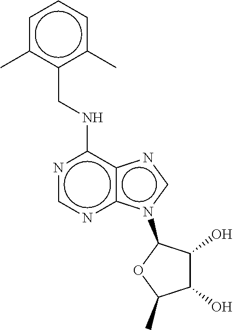 | 1 | (2,6- dimethylphenyl)meth- anamine | 7 | 0.792 | 369.9 | 370.18 |
| 334 | 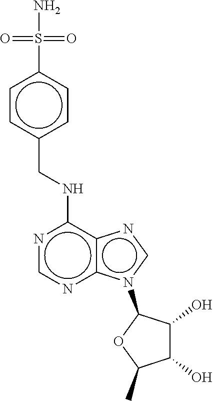 | 1 | 4- (aminomethyl)ben- zenesulfonamide | 7 | 0.789 | 420.9 | 421.12 |
| 352 | 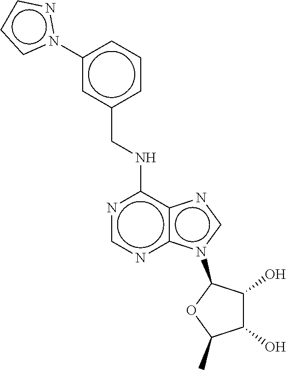 | 1 | (3-(1H-pyrazol-1- yl)phenyl)methana- mine | 7 | 0.757 | 407.9 | 408.17 |
| 370 | 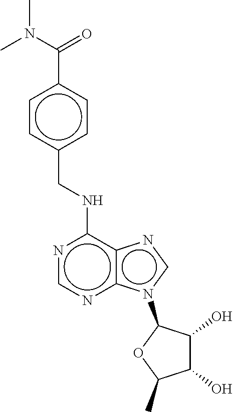 | 1 | 4-(aminomethyl)- N,N- dimethylbenzamide | 7 | 0.695 | 413.1 | 413.19 |
| 374 | 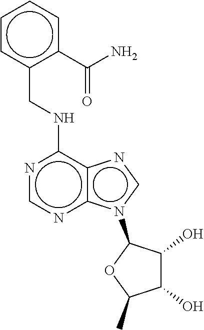 | 1 | 2- (aminomethyl)ben- zamide | 7 | 0.704 | 385.1 | 385.15 |
| 376 | 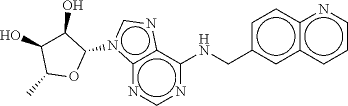 | 1 | quinolin-6- ylmethanamine | 7 | 0.743 | 393.1 | 393.16 |
| 378 | 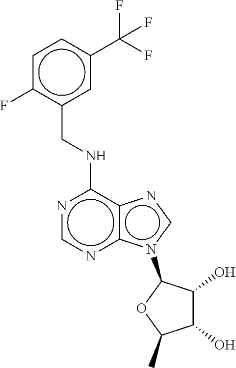 | 1 | (2-fluoro-5- (trifluoromethyl)phe- nyl)methanamine | 7 | 0.825 | 428.0 | 428.13 |
| 384 | 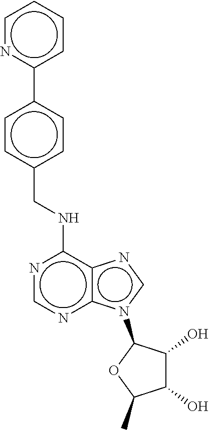 | 1 | (4-(pyridin-2- yl)phenyl)methana- mine | 7 | 0.625 | 419.1 | 419.18 |
| 385 | 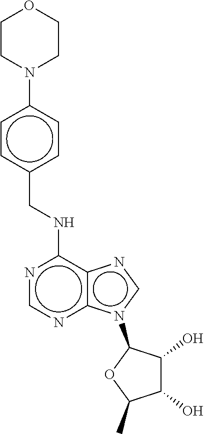 | 1 | (4- morpholinophenyl) methanamine | 7 | 0.674 | 427.2 | 427.20 |
| 386 | 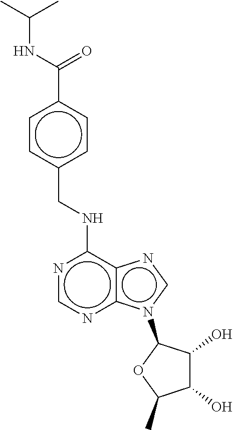 | 1 | 4-(aminomethyl)- N- isopropylbenzamide | 7 | 0.726 | 427.1 | 427.20 |
| 388 | 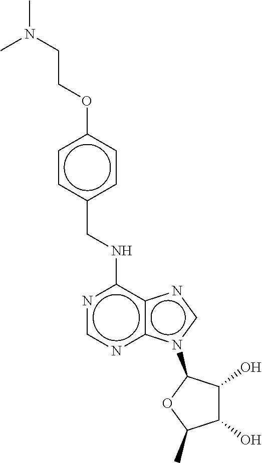 | 1 | 2-(4- (aminomethyl)phe- noxy)-N,N- dimethylethan-1- amine | 7 | 0.788 | 429.1 | 429.22 |
| 389 | 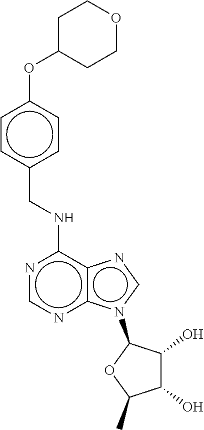 | 1 | (4-((tetrahydro-2H- pyran-4- yl)oxy)phenyl)meth- anamine | 7 | 0.778 | 442.3 | 442.20 |

TABLE 5

| | | Inter- | | | | | Calc- |
|---|---|---|---|---|---|---|---|
| | | mediate | | General | LCMS | LCMS | ulated |
| Comp. | | com- | | pro- | Rt | (M + | (M + |
| No. | Structure | pound | Alcohol | cedure | (min) | H)+ | H)+ |
| 173 | 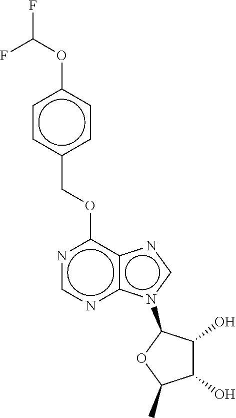 | 1 | (4- (difluoro- methoxy) phenyl) methanol | 5 | 0.837 | 409.2 | 409.12 |
| 174 | 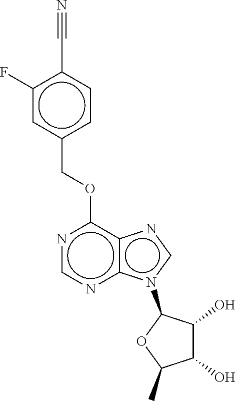 | 1 | 2-fluoro-4- (hydroxymethyl) benzonitrile | 5 | 0.809 | 386.1 | 386.12 |
| 179 | 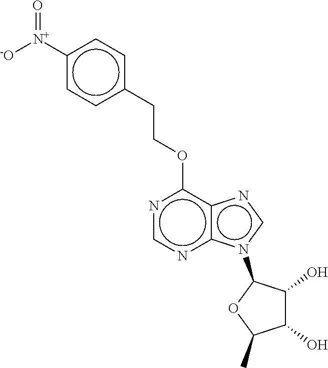 | 1 | 2-(4- nitrophenyl)ethan- 1-o1 | 5 | 0.824 | 402.2 | 402.13 |
| 180 | 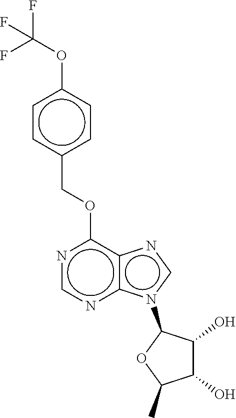 | 1 | (4- (trifluoromethoxy) phenyl)methanol | 5 | 0.824 | 427.1 | 427.12 |
| 182 | 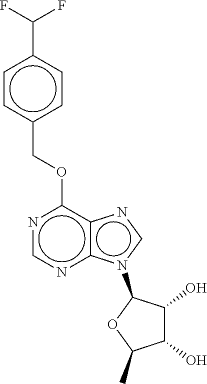 | 1 | (4- (difluoromethyl) phenyl)methanol | 5 | 0.841 | 393.2 | 393.13 |
| 187 | 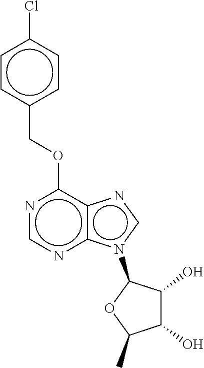 | 1 | (4- chlorophenyl) methanol | 5 | 0.855 | 377.2 | 377.09 |
| 193 | 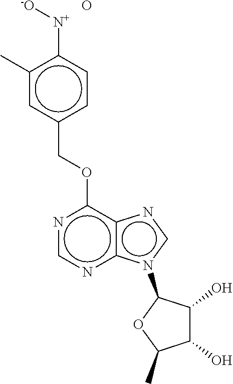 | 1 | (3-methyl-4- nitrophenyl) methanol | 5 | 0.833 | 402.1 | 402.13 |
| 200 | 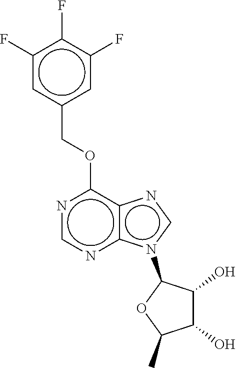 | 1 | (3,4,5- trifluorophenyl) methanol | 5 | 0.867 | 397.0 | 397.10 |
| 201 | 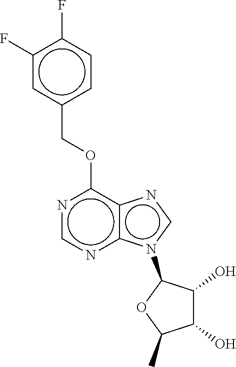 | 1 | (3,4- difluorophenyl) methanol | 5 | 0.827 | 379.3 | 379.11 |
| 202 | 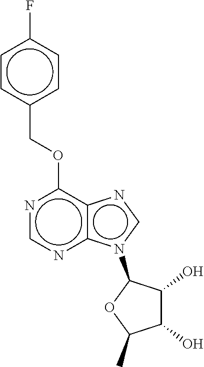 | 1 | (4- fluorophenyl) methanol | 5 | 0.813 | 361.1 | 361.12 |
| 204 | 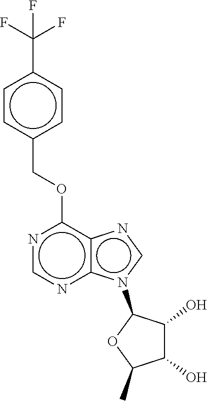 | 1 | (4- (trifluoromethyl) phenyl)methanol | 5 | 0.873 | 411.2 | 411.12 |
| 205 | 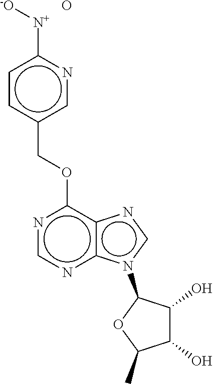 | 1 | (6-nitropyridin-3- yl)methanol | 5 | 0.733 | 389.1 | 389.11 |
| 206 | 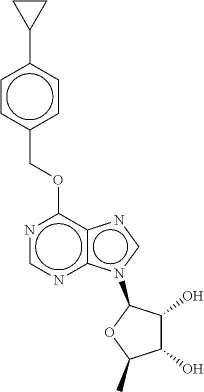 | 1 | (4- cyclopropylphenyl) methanol | 5 | 0.899 | 383.1 | 383.16 |
| 211 | 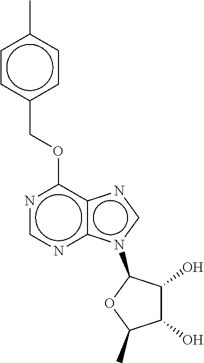 | 1 | p-tolylmethanol | 5 | 0.874 | 357.0 | 357.15 |
| 217 | 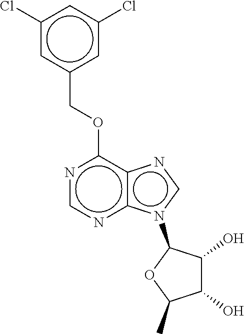 | 1 | (3,5- dichlorophenyl) methanol | 5 | 0.897 | 411.2 | 411.05 |
| 226 | 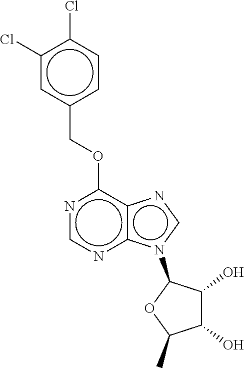 | 1 | (3,4- dichlorophenyl) methanol | 5 | 0.902 | 411.0 | 411.05 |
| 230 | 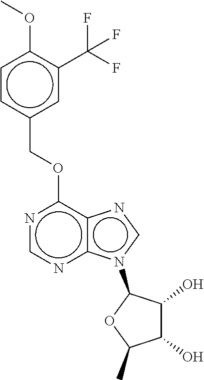 | 1 | (4-methoxy-3- (trifluoromethyl) phenyl)methanol | 5 | 0.903 | 441.1 | 441.13 |
| 231 | 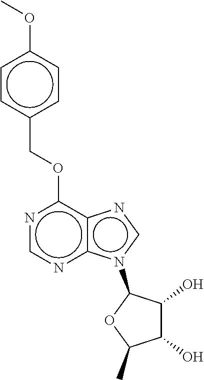 | 1 | (4- methoxyphenyl) methanol | 5 | 0.845 | 373.1 | 373.14 |
| 234 | 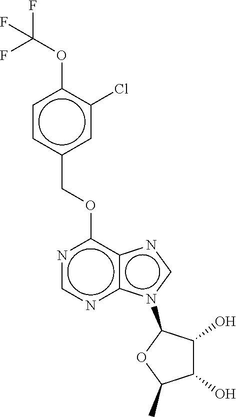 | 1 | (3-chloro-4- (trifluoromethoxy) phenyl)methanol | 5 | 0.916 | 461.2 | 461.08 |
| 235 | 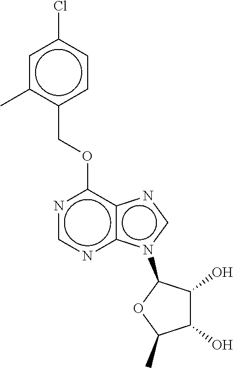 | 1 | (4-chloro-2- methylphenyl) methanol | 5 | 0.891 | 391.1 | 391.11 |
| 236 | 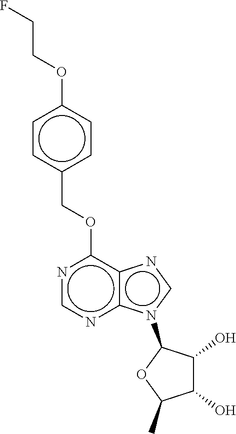 | 1 | (4-(2- fluoroethoxy) phenyl)methanol | 5 | 0.846 | 405.1 | 405.15 |
| 238 | 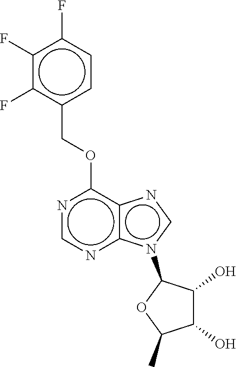 | 1 | (2,3,4- trifluorophenyl) methanol | 5 | 0.856 | 397.0 | 397.10 |
| 241 | 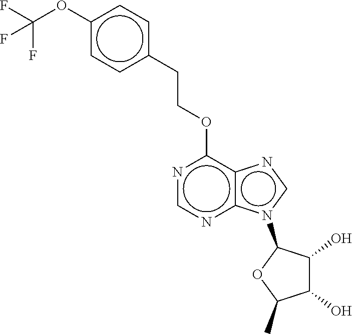 | 1 | 2-(4- (trifluoromethoxy) phenyl)ethan-1-ol | 5 | 0.901 | 441.1 | 441.13 |
| 245 | 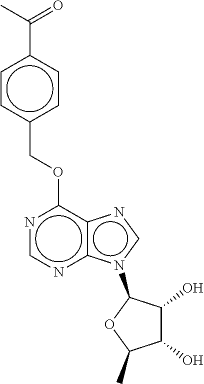 | 1 | 1-(4- (hydroxymethyl) phenyl)ethan-1-one | 5 | 0.790 | 384.9 | 385.14 |
| 246 | 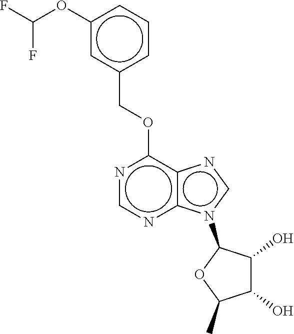 | 1 | (3- (difluoromethoxy) phenyl)methanol | 5 | 0.782 | 409.1 | 409.12 |
| 249 | 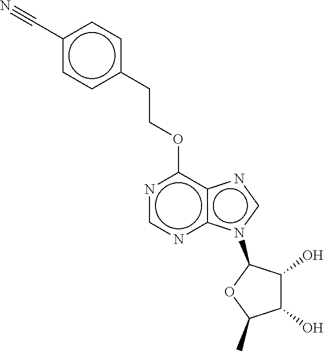 | 1 | 4-(2- hydroxyethyl) benzonitrile | 5 | 0.792 | 382.2 | 382.14 |
| 250 | 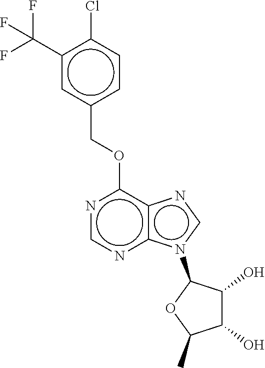 | 1 | (4-chloro-3- (trifluoromethyl) phenyl)methanol | 5 | 0.905 | 445.1 | 445.08 |
| 260 | 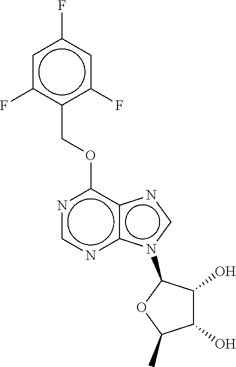 | 1 | (2,4,6- trifluorophenyl) methanol | 5 | 0.750 | 397.1 | 397.10 |
| 269 | 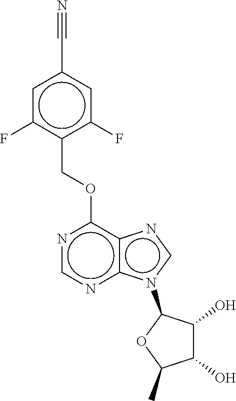 | 1 | 3,5-difluoro-4- (hydroxymethyl) benzonitrile | 5 | 0.816 | 404.2 | 404.11 |
| 274 | 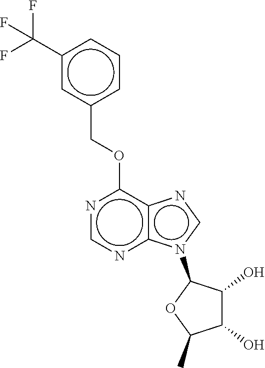 | 1 | (3- (trifluoromethyl) phenyl)methanol | 5 | 0.862 | 410.9 | 411.12 |
| 275 | 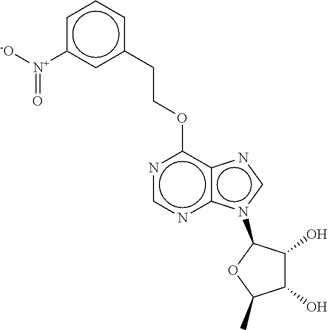 | 1 | 2-(3- nitrophenyl)ethan- 1-ol | 5 | 0.833 | 402.1 | 402.13 |
| 281 | 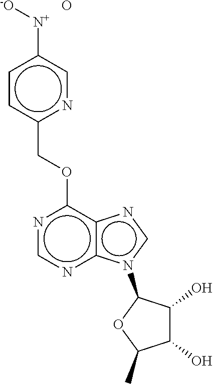 | 1 | (5-nitropyridin-2- yl)methanol | 5 | 0.748 | 389.0 | 389.11 |
| 291 | 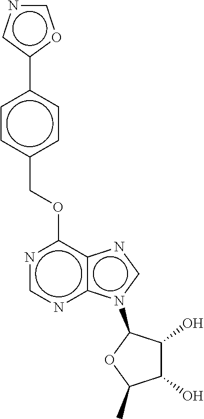 | 1 | (4-(oxazol-5- yl)phenyl)methanol | 5 | 0.816 | 410.1 | 410.14 |
| 293 | 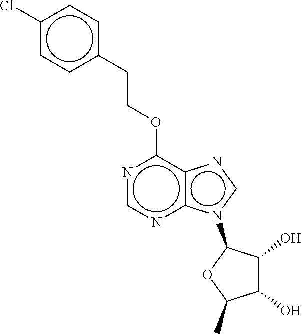 | 1 | 2-(4- chlorophenyl)ethan [symbol] 1-o1 | 5 | 0.902 | 391.1 | 391.11 |
| 307 | 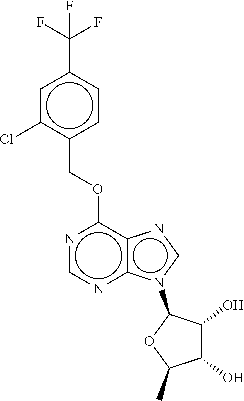 | 1 | (2-chloro-4- (trifluoromethyl) phenyl)methanol | 5 | 0.912 | 445.2 | 445.08 |
| 313 | 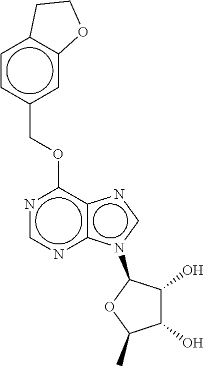 | 1 | (2,3- dihydrobenzofuran- 6-yl)methanol | 5 | 0.816 | 384.9 | 385.14 |
| 314 | 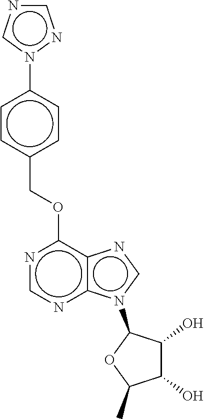 | 1 | (4-(1H-1,2,4- triazol-1- yl)phenyl)methanol | 5 | 0.773 | 410.0 | 410.15 |
| 316 | 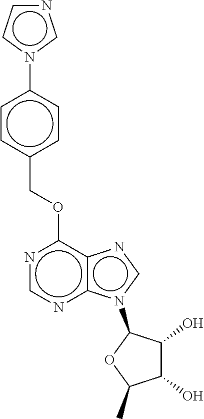 | 1 | (4-(1H-imidazol-1- yl)phenyl)methanol | 5 | 0.768 | 409.1 | 409.15 |
| 321 | 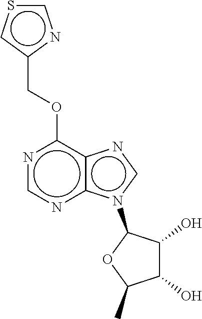 | 1 | thiazol-4- ylmethanol | 5 | 0.705 | 350.2 | 350.08 |
| 323 | 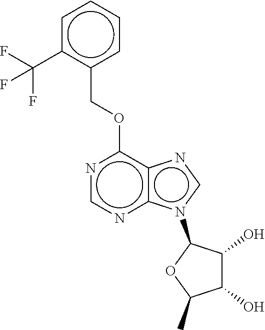 | 1 | (2- (trifluoromethyl) phenyl)methanol | 5 | 0.874 | 411.1 | 411.12 |
| 325 | 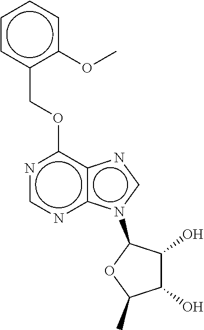 | 1 | (2- methoxyphenyl) methanol | 5 | 0.817 | 373.1 | 373.14 |
| 328 | 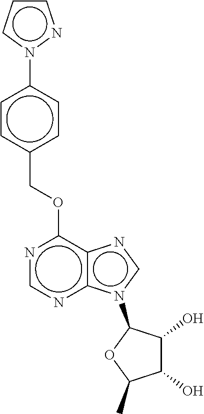 | 1 | (4-(1H-pyrazol-1- yl)phenyl)methanol | 5 | 0.831 | 409.2 | 409.15 |
| 332 | 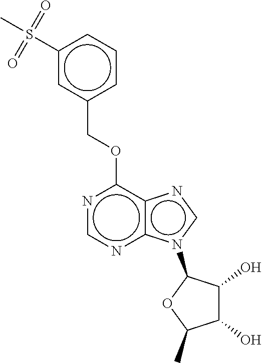 | 1 | (3- (methylsulfonyl) phenyl)methanol | 5 | 0.753 | 421.1 | 421.11 |
| 333 | 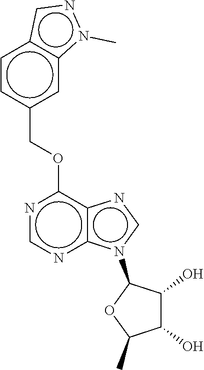 | 1 | (1-methyl-1H- indazol-6- yl)methanol | 5 | 0.789 | 397.1 | 397.15 |
| 338 | 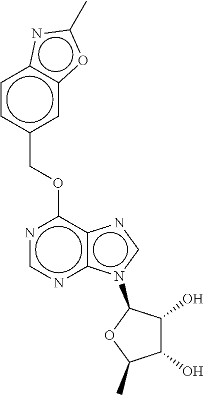 | 1 | (2- methylbenzo[d] oxazol-6-yl) methanol | 5 | 0.800 | 398.3 | 398.14 |
| 340 | 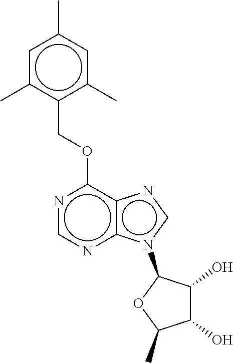 | 1 | mesitylmethanol | 5 | 0.903 | 385.2 | 385.18 |
| 346 | 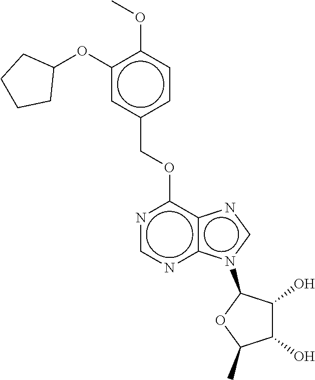 | 1 | (3- (cyclopentyloxy)-4- methoxyphenyl) methanol | 5 | 0.890 | 457.1 | 457.20 |
| 358 | 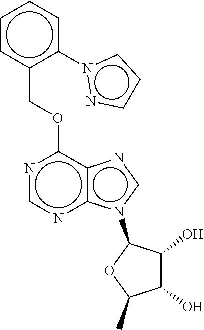 | 1 | (2-(1H-pyrazol-1- yl)phenyl)methanol | 5 | 0.819 | 409.1 | 409.15 |
| 362 | 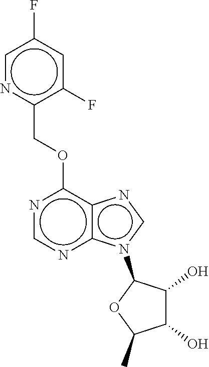 | 1 | (3,5- difluoropyridin-2- yl)methanol | 5 | 0.748 | 380.0 | 380.11 |
| 363 | 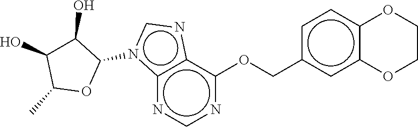 | 1 | (2,3- dihydrobenzo [b][1,4] dioxin-6- yl)methanol | 5 | 0.729 | 401.1 | 401.14 |
| 364 | 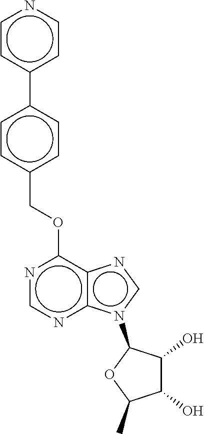 | 1 | (4-(pyridin-4- yl)phenyl)methanol | 5 | 0.729 | 420.2 | 420.16 |
| 367 | 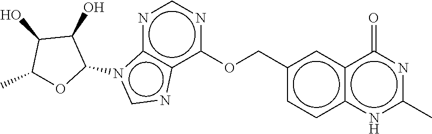 | 1 | 6-(hydroxymethyl)- 2- methylquinazolin- 4(1H)-one | 5 | 0.683 | 425.1 | 425.15 |
| 372 | 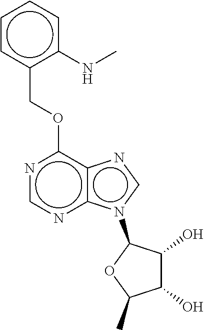 | 1 | (2- (methylamino) phenyl)methanol | 5 | 0.628 | 372.0 | 372.16 |
| 375 | 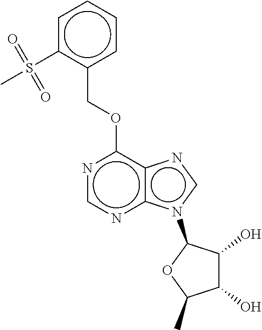 | 1 | (2- (methylsulfonyl) phenyl)methanol | 5 | 0.768 | 420.9 | 421.11 |
| 377 | 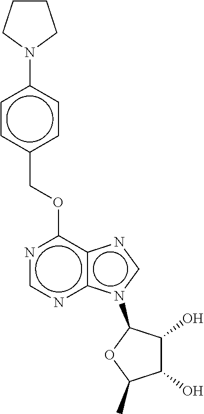 | 1 | (4-(pyrrolidin-1- yl)phenyl)methanol | 5 | 0.728 | 411.3 | 412.19 |
| 381 | 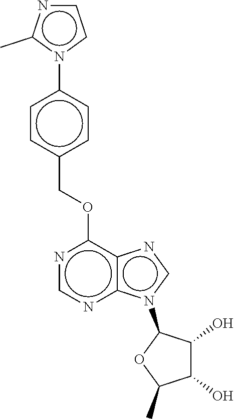 | 1 | (4-(2-methyl-1H- imidazol-1- yl)phenyl)methanol | 5 | 0.666 | 423.1 | 423.17 |
| 382 | 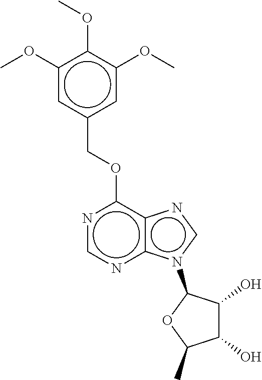 | 1 | (3,4,5- trimethoxyphenyl) methanol | 5 | 0.782 | 433.1 | 433.16 |
| 383 | 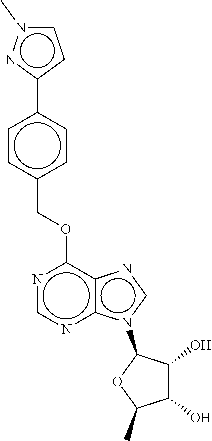 | 1 | (4-(1-methyl-1H- pyrazol-3- yl)phenyl)methanol | 5 | 0.824 | 423.2 | 423.17 |

[symbol]

indicates data missing or illegible when filed

TABLE 6

| | | Inter- | Thiol | General | LCMS | LCMS | Calcu- |
|---|---|---|---|---|---|---|---|
| Comp. | | mediate | (S-alkyl | pro- | Rt | (M + | lated |
| No. | Structure | compound | ethanethioate) | cedure | (min) | H)+ | (M + H)+ |
| 147 | 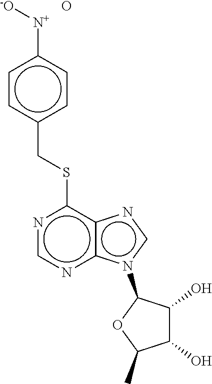 | 1 | (4- nitrophenyl)meth- anethiol | 8 | 0.839 | 404.0 | 404.10 |
| 176 | 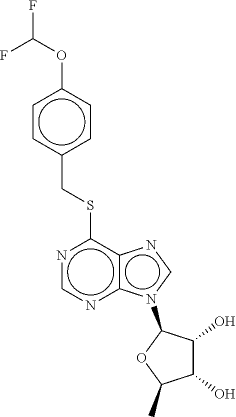 | 1 | S-(4- (difluorometh- oxy)benzyl) ethanethioate | 8 | 0.864 | 425.1 | 425.10 |
| 178 | 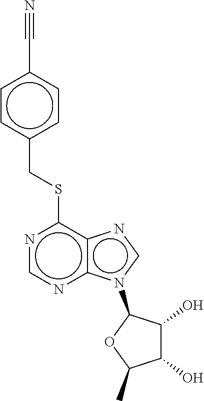 | 1 | 4- (mercaptometh- yl)benzonitrile | 8 | 0.861 | 384.0 | 384.11 |
| 179 | 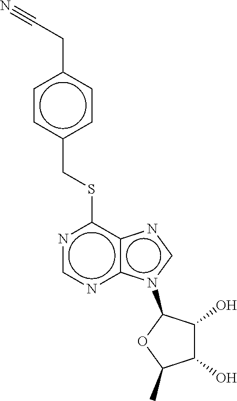 | 1 | S-(4- (cyanomethyl) benzyl) ethanethioate | 8 | 0.820 | 397.9 | 398.12 |
| 186 | 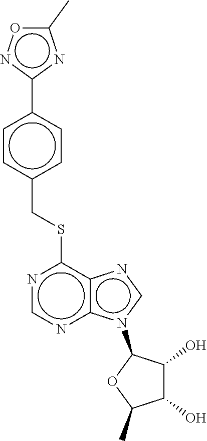 | 1 | (4-(5-methyl- 1,2,4- oxadiazol-3- yl)phenyl)meth- anethiol | 8 | 0.830 | 441.1 | 441.13 |
| 207 | 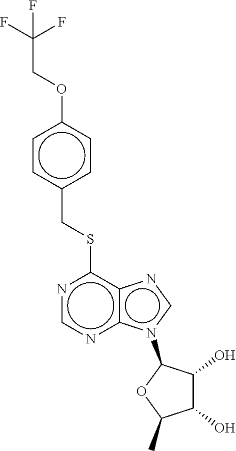 | 1 | (4-(2,2,2- trifluoroethoxy) phenyl)methane- thiol | 8 | 0.936 | 457.1 | 457.11 |
| 224 | 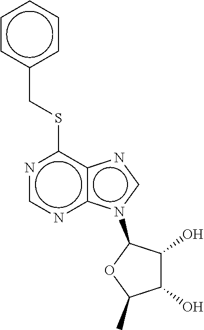 | 1 | phenylmethane- thiol | 8 | 0.738 | 359.1 | 395.11 |
| 242 | 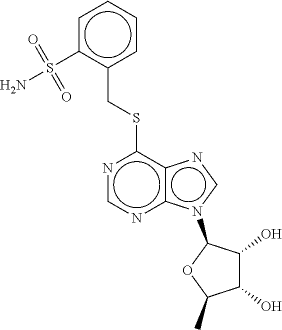 | 1 | 2- (mcercaptometh- yl)benzenesul- fonamide | 8 | 0.757 | 438.0 | 438.08 |
| 251 | 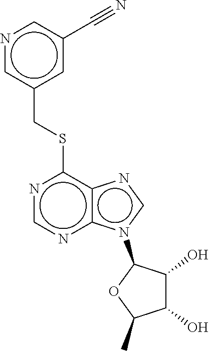 | 1 | 5- (mercaptometh- yl)nicotinonitrile | 8 | 0.749 | 385.0 | 385.10 |
| 257 | 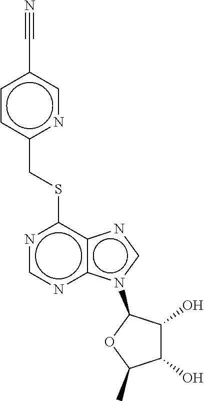 | 1 | 6- (mcercaptometh- yl)nicotinonitrile | 8 | 0.749 | 385.0 | 385.10 |
| 270 | 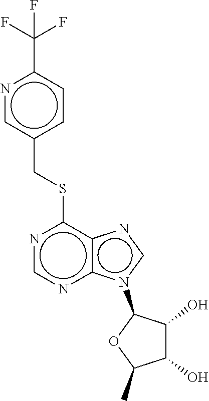 | 1 | (6- (trifluorometh- yl)pyridin-3- yl)methanethiol | 8 | 0.813 | 428.0 | 428.09 |
| 272 | 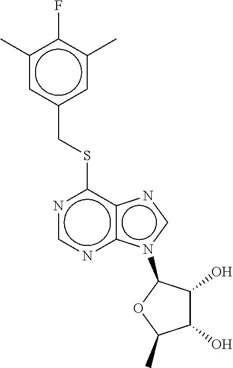 | 1 | (4-fluoro-3,5- dimethylphenyl) methanethiol | 8 | 0.906 | 405.1 | 405.13 |
| 273 | 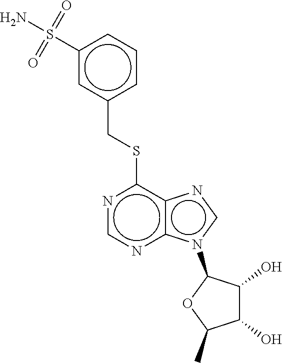 | 1 | 3- (mercaptometh- yl)benzenesul- fonamide | 8 | 0.741 | 438.0 | 438.08 |
| 289 | 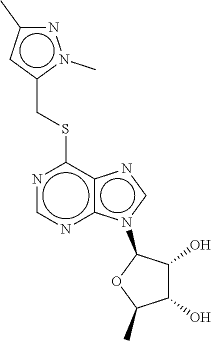 | 1 | (1,3-dimethyl- 1H-pyrazol-5- yl)methanethiol | 8 | 0.735 | 377.2 | 377.13 |
| 297 | 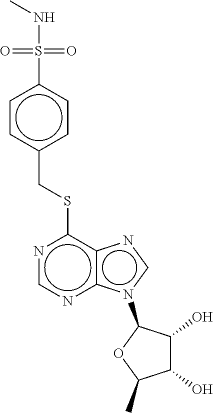 | 1 | 4- (mercaptometh- yl)-N- methylbenzene- sulfonamide | 8 | 0.748 | 452.0 | 452.10 |
| 300 | 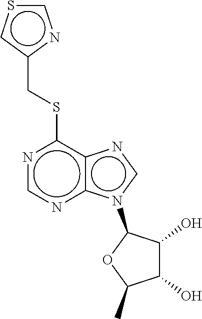 | 1 | thiazol-4- ylmethanethiol | 8 | 0.728 | 366.0 | 366.06 |
| 306 | 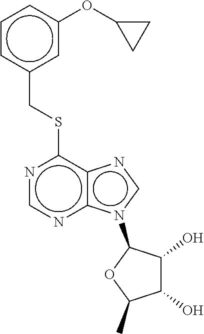 | 1 | (3- cyclopropoxyphe- nyl)methane- thiol | 8 | 0.903 | 415.1 | 415.14 |
| 308 | 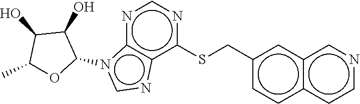 | 1 | isoquinolin-7- ylmethanethiol | 8 | 0.803 | 410.1 | 410.12 |
| 310 | 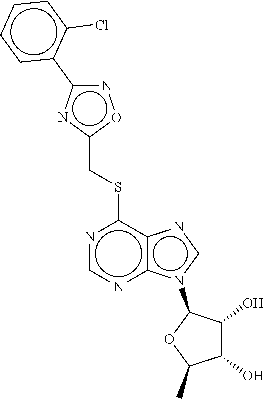 | 1 | (3-(2- chlorophenyl)- 1,2,4- oxadiazol-5- yl)methanethiol | 8 | 0.845 | 461.0 | 461.07 |
| 311 | 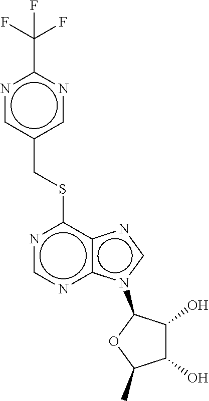 | 1 | (2- (trifluorometh- yl)pyrimidin-5- yl)methanethiol | 8 | 0.798 | 429.0 | 429.09 |
| 337 | 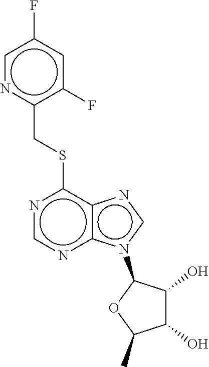 | 1 | (3,5- difluoropyridin- 2- yl)methanethiol | 8 | 0.789 | 396.0 | 396.09 |
| 342 | 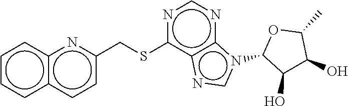 | 1 | quinolin-2- ylmethanethiol | 8 | 0.724 | 410.2 | 410.12 |
| 348 | 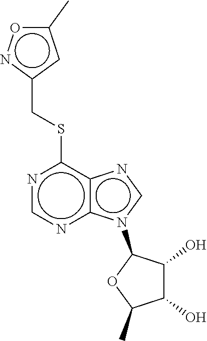 | 1 | (5- methylisoxazol- 3- yl)methanethiol | 8 | 0.754 | 364.0 | 364.10 |
| 359 | 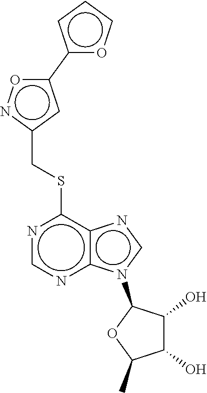 | 1 | (5-(furan-2- yl)isoxazol-3- yl)methanethiol | 8 | 0.830 | 416.1 | 416.10 |
| 365 | 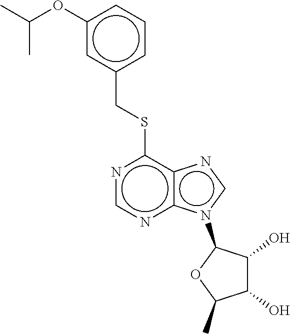 | 1 | (3- isopropoxyphe- nyl)methanethi- ol | 8 | 0.861 | 417.1 | 417.15 |
| 366 | 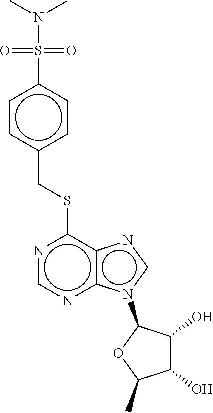 | 1 | 4- (mercaptometh- yl)-N,N- dimethylbenzene- sulfonamide | 8 | 0.826 | 466.1 | 466.11 |
| 390 | 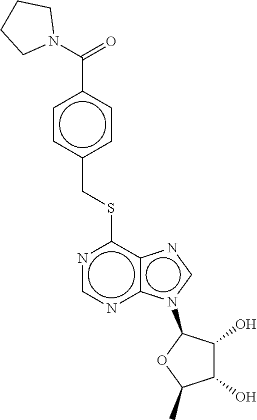 | 1 | (4- (mercaptometh- yl)phenyl)(py- rrolidin-1- yl)methanone | 8 | 0.806 | 456.1 | 456.16 |
| 391 | 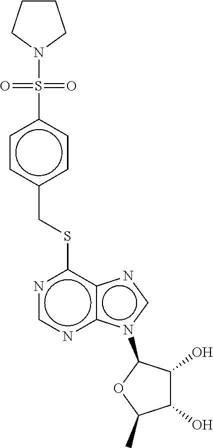 | 1 | (4-(pyrrolidin- 1- ylsulfonyl)phe- nyl)methanethi- ol | 8 | 0.826 | 492.0 | 492.13 |
| 392 | 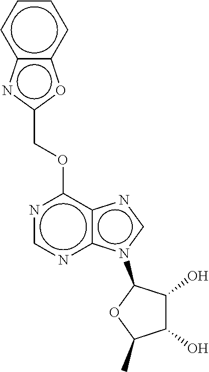 | 1 | benzo[d]oxazol- 2- ylmethanethiol | 8 | 0.811 | 399.9 | 400.10 |
| 393 | 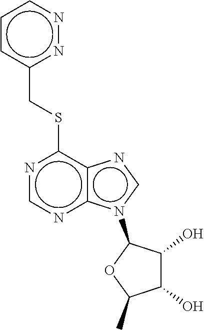 | 1 | pyridazin-3- ylmethanethiol | 8 | 0.720 | 361.1 | 361.10 |
| 394 | 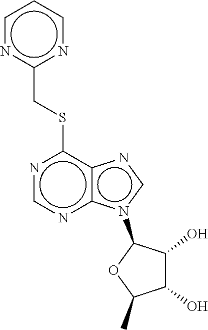 | 1 | pyrimidin-2- ylmethanethiol | 8 | 0.675 | 361.0 | 361.10 |
| 395 | 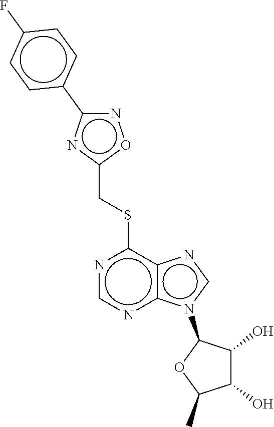 | 1 | (3-(4- fluorophenyl)- 1,2,4- oxadiazol-5- yl)methanethiol | 8 | 0.861 | 445.0 | 445.10 |
| 396 | 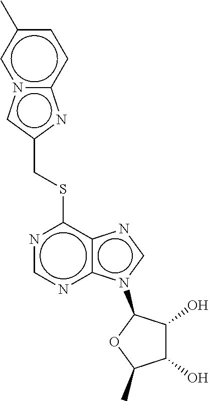 | 1 | (6- methylimidazo [1,2-a]pyridin- 2- yl)methanethiol | 8 | 0.663 | 413.0 | 413.3 |
| 397 | 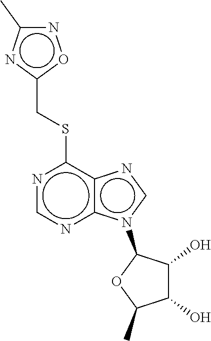 | 1 | (3-methyl- 1,2,4- oxadiazol-5- yl)methanethiol | 8 | 0.732 | 365.1 | 365.10 |
| 398 | 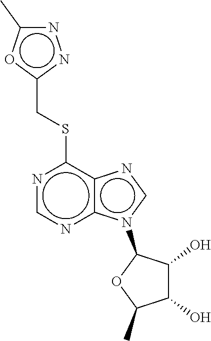 | 1 | (5-methyl- 1,3,4- oxadiazol-2- yl)methanethiol | 8 | 0.663 | 365.0 | 365.10 |

TABLE 7
Analytical data of the compounds described in Tables 2 to 6 are shown below.

| Compound | |
|---|---|
| number | NMR description |
| 4 | 1H NMR (400 MHz, DMSO-d6) δ = 8.26 (br d, J = 8.6 Hz, 2H), 8.18 (s, 1H), |
| | 7.71 (br d, J = 8.2 Hz, 2H), 6.96 (br s, 2H), 5.72-5.67 (m, 3H), 5.28 (br d, J = |
| | 4.6 Hz, 1H), 5.05 (br d, J = 5.0 Hz, 2H), 4.15-4.09 (m, 1H), 3.91-3.83 |
| | (m, 1H), 1.26 (br d, J = 6.4 Hz, 3H) |
| 5 | 1H NMR (400 MHz, DMSO-d6) δ = 8.20 (s, 1H), 7.48 (br d, J = 7.0 Hz, 2H), |
| | 7.42-7.37 (m, 3H), 7.02 (br d, J = 6.8 Hz, 1H), 5.77 (d, J = 4.0 Hz, 1H), 5.52 |
| | (s, 2H), 5.25 (d, J = 5.2 Hz, 1H), 5.06 (br dd, J = 5.4, 13.4 Hz, 2H), 4.21- |
| | 4.11 (m, 2H), 3.87 (t, J = 6.0 Hz, 1H), 1.96-1.92 (m, 2H), 1.68 (br s, 2H), |
| | 1.53 (br s, 4H), 1.25 (d, J = 6.2 Hz, 3H) |
| 6 | 1H NMR (400 MHz, DMSO-d6) δ = 8.25 (d, J = 8.6 Hz, 2H), 8.18 (s, 1H), |
| | 7.71 (d, J = 8.4 Hz, 2H), 7.09 (d, J = 6.8 Hz, 1H), 5.78 (d, J = 4.2 Hz, 1H), |
| | 5.69 (s, 2H), 5.26 (d, J = 5.4 Hz, 1H), 5.10-5.04 (m, 2H), 4.25-4.18 (m, |
| | 1H), 4.13 (q, J = 5.8 Hz, 1H), 3.88 (q, J = 6.2 Hz, 1H), 2.01-1.93 (m, 2H), |
| | 1.72-1.66 (m, 2H), 1.59-1.52 (m, 4H), 1.25 (d, J = 6.2 Hz, 3H) |
| 7 | 1H NMR (400 MHz, DMSO-d6) δ = 8.23 (s, 1H), 7.55 (d, J = 8.6 Hz, 2H), |
| | 7.42-7.38 (m, 1H), 7.31-7.14 (m, 5H), 7.04-6.96 (m, 2H), 6.94-6.89 (m, |
| | 1H), 5.75 (d, J = 4.4 Hz, 1H), 5.52 (s, 2H), 5.29 (d, J = 5.4 Hz, 1H), 5.12- |
| | 5.02 (m, 2H), 4.17 (t, J = 5.8 Hz, 2H), 4.12-4.08 (m, 1H), 3.93-3.84 (m, |
| | 1H), 3.71 (q, J = 5.2 Hz, 2H), 1.26 (d, J = 6.4 Hz, 3H) |
| 8 | 1H NMR (400 MHz, DMSO-d6) δ = 8.22 (s, 1H), 7.81-7.75 (m, 1H), 7.53- |
| | 7.51 (m, 1H), 7.40 (s, J = 148 Hz, 1H), 7.35-7.30 (m, 4H), 7.26-7.15 (m, |
| | 4H), 5.80-5.77 (m, 1H), 5.52-5.49 (m, 2H), 5.32-5.29 (m, 1H), 5.17-5.12 |
| | (m, 1H), 5.08-5.05 (m, 1H), 4.59-4.56 (m, 2H), 4.14-4.10 (m, 1H), 3.92- |
| | 3.86 (m, 1H), 1.26 (d, J = 6.4 Hz, 3H). |
| 9 | 1H NMR (400 MHz, DMSO-d6) δ = 8.25 (d, J = 8.6 Hz, 2H), 8.21 (s, 1H), |
| | 7.71 (d, J = 8.8 Hz, 2H), 7.45 (d, J = 6.8 Hz, 1H), 5.75 (d, J = 4.4 Hz, 1H), |
| | 5.70 (s, 2H), 5.29 (d, J = 5.2 Hz, 1H), 5.13 (t, J = 4.4 Hz, 1H), 5.08 (d, J = |
| | 5.6 Hz, 1H), 4.48-4.39 (m, 1H), 4.14 (q, J = 5.6 Hz, 1H), 3.89 (t, J = 6.2 Hz, |
| | 1H), 2.59 (br s, 1H), 2.26-2.13 (m, 4H), 1.85 (dt, J = 4.4, 8.4 Hz, 1H), 1.25 |
| | (d, J = 6.4 Hz, 3H) |
| 10 | 1H NMR (400 MHz, DMSO-d6) δ = 8.25 (d, J = 8.6 Hz, 2H), 8.19 (s, 1H), |
| | 7.70 (d, J = 8.8 Hz, 2H), 7.17 (d, J = 7.6 Hz, 1H), 5.76 (d, J = 4.4 Hz, 1H), |
| | 5.70 (s, 2H), 5.27 (d, J = 5.4 Hz, 1H), 5.15-5.09 (m, 1H), 5.07 (d, J = 5.8 |
| | Hz, 1H), 4.14 (q, J = 5.6 Hz, 1H), 4.05-3.96 (m, 1H), 3.92-3.84 (m, 1H), |
| | 2.09-1.96 (m, 6H), 1.72-1.62 (m, 2H), 1.25 (d, J = 6.4 Hz, 3H) |
| 11 | 1H NMR (400 MHz, DMSO-d6) δ = 8.20 (s, 1H), 7.54 (d, J = 8.6 Hz, 2H), |
| | 7.41-7.04 (t, J = 74 Hz, 1H), 7.19 (d, J = 8.6 Hz, 2H), 7.02 (d, J = 7.0 Hz, |
| | 1H), 5.77 (d, J = 4.4 Hz, 1H), 5.51 (s, 2H), 5.25 (d, J = 5.2 Hz, 1H), 5.10- |
| | 5.02 (m, 2H), 4.23-4.09 (m, 2H), 3.87 (quin, J = 6.2 Hz, 1H), 1.94 (br dd, J = |
| | 2.8, 6.6 Hz, 2H), 1.72-1.63 (m, 2H), 1.59-1.50 (m, 4H), 1.25 (d, J = 6.4 |
| | Hz, 3H) |
| 14 | 1H NMR (400 MHz, DMSO-d6) δ = 8.42 (d, J = 2.0 Hz, 1H), 8.21 (s, 1H), |
| | 8.06 (dd, J = 2.4, 8.5 Hz, 1H), 7.90-7.52 (m, 1H), 7.12 (d, J = 8.4 Hz, 1H), |
| | 7.05 (br d, J = 6.8 Hz, 1H), 5.77 (d, J = 4.1 Hz, 1H), 5.54 (s, 2H), 5.25 (d, J = |
| | 5.2 Hz, 1H), 5.09-5.03 (m, 2H), 4.23-4.09 (m, 2H), 3.87 (quin, J = 6.2 |
| | Hz, 1H), 1.94 (br d, J = 2.8 Hz, 2H), 1.71-1.64 (m, 2H), 1.53 (br s, 4H), 1.25 |
| | (d, J = 6.4 Hz, 3H) |
| 15 | 1H NMR (400 MHz, DMSO-d6) δ = 8.42 (d, J = 5.2 Hz, 1H), 8.17 (s, 1H), |
| | 7.28 (s, 1H), 7.20 (d, J = 4.9 Hz, 1H), 7.09 (d, J = 6.8 Hz, 1H), 5.78 (d, J = |
| | 4.0 Hz, 1H), 5.54 (s, 2H), 5.27 (d, J = 5.4 Hz, 1H), 5.11-5.03 (m, 2H), 4.26- |
| | 4.18 (m, 1H), 4.13 (q, J = 5.6 Hz, 1H), 3.88 (quin, J = 6.2 Hz, 1H), 2.46 (s, |
| | 3H), 2.03-1.90 (m, 2H), 1.76-1.64 (m, 2H), 1.62-1.49 (m, 4H), 1.25 (d, J = |
| | 6.4 Hz, 3H) |
| 16 | 1H NMR (400 MHz, DMSO-d6) δ = 8.25 (d, J = 8.8 Hz, 2H), 8.19 (s, 1H), |
| | 7.72 (d, J = 8.8 Hz, 2H), 7.62 (s, 1H), 7.53 (t, J = 5.6 Hz, 1H), 7.39 (s, 1H), |
| | 5.73-5.70 (m, 3H), 5.26 (d, J = 5.4 Hz, 1H), 5.09 (q, J = 5.4 Hz, 1H), 5.05 |
| | (d, J = 5.8 Hz, 1H), 4.39 (d, J = 5.4 Hz, 2H), 4.10 (q, J = 5.8 Hz, 1H), 3.89- |
| | 3.82 (m, 1H), 3.78 (s, 3H), 1.24 (d, J = 6.4 Hz, 3H) |
| 17 | 1H NMR (400 MHz, DMSO-d6) δ = 9.53 (s, 1H), 8.28-8.23 (m, 3H), 8.00 |
| | (s, 1H), 7.73 (d, J = 8.8 Hz, 2H), 7.56 (s, 1H), 5.89 (d, J = 4.0 Hz, 1H), 5.76 |
| | (s, 2H), 5.38 (d, J = 5.0 Hz, 1H), 5.25 (br d, J = 4.6 Hz, 1H), 5.10 (d, J = 5.4 |
| | Hz, 1H), 4.20-4.15 (m, 1H), 3.93 (t, J = 6.2 Hz, 1H), 3.83 (s, 3H), 1.27 (d, J = |
| | 6.4 Hz, 3H) |
| 18 | 1H NMR (400 MHz, DMSO-d6) δ = 8.25 (d, J = 8.4 Hz, 2H), 8.21-8.20 (m, |
| | 1H), 7.73-7.70 (m, 2H), 7.39-7.36 (m, 1H), 5.81-5.79 (m, 1H), 5.71-5.69 |
| | (m, 2H), 5.27 (t, J = 5.6 Hz, 1H), 5.11-5.06 (m, 2H), 4.47-4.43 (m, 1H), |
| | 4.16-4.12 (m, 1H), 3.88 (br dd, J = 5.6, 9.0 Hz, 3H), 3.74-3.70 (m, 1H), |
| | 3.66 (dd, J = 3.8, 8.8 Hz, 1H), 2.24-2.18 (m, 1H), 2.01-1.95 (m, 1H), 1.25 |
| | (br d, J = 6.0 Hz, 3H) |
| 19 | 1H NMR (400 MHz, DMSO-d6) δ = 8.25 (d, J = 8.8 Hz, 2H), 8.19 (s, 1H), |
| | 7.72 (d, J = 8.8 Hz, 2H), 7.23-7.19 (m, 1H), 5.69 (s, 2H), 5.66 (d, J = 4.4 |
| | Hz, 1H), 5.30 (d, J = 5.2 Hz, 1H), 5.13-5.08 (m, 1H), 5.06 (d, J = 5.8 Hz, |
| | 1H), 4.11 (d, J = 5.8 Hz, 1H), 3.89-3.85 (m, 1H), 2.90 (d, J = 4.6 Hz, 3H), |
| | 1.25 (d, J = 6.4 Hz, 3H) |
| 20 | 1H NMR (400 MHz, DMSO-d6) δ = 8.25 (d, J = 8.8 Hz, 2H), 8.19-8.17 (m, |
| | 1H), 7.71 (d, J = 8.8 Hz, 2H), 7.24 (t, J = 5.4 Hz, 1H), 5.71-5.68 (m, 3H), |
| | 5.30-5.27 (m, 1H), 5.13-5.09 (m, 1H), 5.06 (d, J = 5.8 Hz, 1H), 4.15-4.10 |
| | (m, 1H), 3.91-3.84 (m, 1H), 3.43-3.37 (m, 2H), 1.27-1.24 (m, 3H), 1.19 |
| | (t, J = 7.2 Hz, 3H) |
| 21 | 1H NMR (400 MHz, DMSO-d6) δ = 8.25 (d, J = 8.8 Hz, 2H), 8.19 (s, 1H), |
| | 7.71 (d, J = 8.8 Hz, 2H), 7.14 (br t, J = 5.4 Hz, 1H), 5.74 (d, J = 4.4 Hz, 1H), |
| | 5.69 (s, 2H), 5.29 (d, J = 5.2 Hz, 1H), 5.08 (d, J = 5.8 Hz, 1H), 5.07-5.03 |
| | (m, 1H), 4.80 (t, J = 5.4 Hz, 1H), 4.12-4.06 (m, 1H), 3.93-3.85 (m, 1H), |
| | 3.61-3.57 (m, 2H), 3.45-3.40 (m, 2H), 1.26 (d, J = 6.4 Hz, 3H) |
| 22 | 1H NMR (400 MHz, DMSO-d6) δ = 8.25 (d, J = 8.8 Hz, 2H), 8.19 (s, 1H), |
| | 7.71 (d, J = 8.8 Hz, 2H), 7.19 (br t, J = 5.2 Hz, 1H), 5.72 (d, J = 4.4 Hz, 1H), |
| | 5.69 (s, 2H), 5.29 (d, J = 5.4 Hz, 1H), 5.10-5.05 (m, 2H), 4.58 (t, J = 5.4 Hz, |
| | 1H), 4.13-4.07 (m, 1H), 3.92-3.83 (m, 1H), 3.51-3.46 (m, 2H), 3.45-3.39 |
| | (m, 2H), 1.79-1.72 (m, 2H), 1.26 (d, J = 6.4 Hz, 3H) |
| 23 | 1H NMR (400 MHz, DMSO-d6) δ = 8.25 (d, J = 8.8 Hz, 2H), 8.20 (s, 1H), |
| | 7.71 (d, J = 8.8 Hz, 2H), 7.24-7.18 (m, 1H), 5.75 (d, J = 4.6 Hz, 1H), 5.69 |
| | (s, 2H), 5.29 (d, J = 5.4 Hz, 1H), 5.09-5.02 (m, 2H), 4.12-4.05 (m, 1H), |
| | 3.89 (t, J = 6.0 Hz, 1H), 3.56-3.50 (m, 4H), 3.28 (s, 3H), 1.26 (d, J = 6.4 Hz, |
| | 3H) |
| 25 | 1H NMR (400 MHz, DMSO-d6) δ = 8.27-8.23 (m, 2H), 8.17 (s, 1H), 7.73- |
| | 7.69 (m, 2H), 7.06-7.02 (m, 1H), 5.77-5.74 (m, 1H), 5.70-5.67 (m, 2H), |
| | 5.29-5.25 (m, 1H), 5.11-5.04 (m, 2H), 4.15-4.08 (m, 2H), 3.90-3.84 (m, |
| | 1H), 1.26-1.24 (m, 3H), 1.23-1.20 (m, 6H) |
| 26 | 1H NMR (400 MHz, DMSO-d6) δ = 8.25 (d, J = 8.8 Hz, 2H), 8.20 (s, 1H), |
| | 7.73 (d, J = 8.6 Hz, 2H), 7.48 (d, J = 2.4 Hz, 1H), 5.70 (s, 2H), 5.67 (d, J = |
| | 4.2 Hz, 1H), 5.25 (d, J = 5.4 Hz, 1H), 5.09 (q, J = 5.0 Hz, 1H), 5.04 (d, J = |
| | 5.8 Hz, 1H), 4.13 (d, J = 5.8 Hz, 1H), 3.86 (quin, J = 6.2 Hz, 1H), 2.79 (dt, J = |
| | 3.2, 6.6 Hz, 1H), 1.24 (d, J = 6.4 Hz, 3H), 0.74-0.69 (m, 2H), 0.58-0.53 |
| | (m, 2H) |
| 27 | 1H NMR (400 MHz, DMSO-d6) δ = 8.26-8.18 (m, 3H), 7.84 (br t, J =6.0 |
| | Hz, 1H), 7.69 (d, J = 8.8 Hz, 2H), 7.38-7.30 (m, 4H), 7.27-7.21 (m, 1H), |
| | 5.79 (d, J = 4.5 Hz, 1H), 5.68 (s, 2H), 5.32 (d, J = 5.4 Hz, 1H), 5.19-5.12 |
| | (m, 1H), 5.07 (d, J = 5.6 Hz, 1H), 4.60 (d, J = 5.8 Hz, 2H), 4.15-4.07 (m, |
| | 1H), 3.94-3.84 (m, 1H), 1.26 (d, J = 6.2 Hz, 3H) |
| 28 | 1H NMR (400 MHz, DMSO-d6) δ = 8.25 (d, J = 8.6 Hz, 2H), 8.17 (s, 1H), |
| | 7.71 (d, J = 8.6 Hz, 2H), 6.99 (br d, J = 7.8 Hz, 1H), 5.75 (d, J = 4.2 Hz, 1H), |
| | 5.70 (s, 2H), 5.27 (d, J = 5.2 Hz, 1H), 5.10-5.05 (m, 2H), 4.57 (d, J = 4.4 |
| | Hz, 1H), 4.12 (q, J = 5.8 Hz, 1H), 3.87 (quin, J = 6.0 Hz, 1H), 3.76-3.68 (m, |
| | 1H), 3.43-3.39 (m, 1H), 1.92 (br s, 2H), 1.87-1.83 (m, 2H), 1.40-1.27 (m, |
| | 4H), 1.24 (br d, J = 6.2 Hz, 3H) |
| 29 | 1H NMR (400 MHz, DMSO-d6) δ = 8.25 (d, J = 8.6 Hz, 2H), 8.19 (s, 1H), |
| | 7.71 (d, J = 8.6 Hz, 2H), 7.01 (br d, J = 7.6 Hz, 1H), 5.78 (d, J = 4.4 Hz, 1H), |
| | 5.69 (s, 2H), 5.30-5.26 (m, 1H), 5.07 (dd, J = 3.2, 5.2 Hz, 1H), 4.97-4.93 |
| | (m, 1H), 4.82 (t, J = 3.8 Hz, 1H), 4.26-4.20 (m, 1H), 4.17-4.12 (m, 1H), |
| | 4.08-4.03 (m, 1H), 3.90-3.84 (m, 1H), 2.21-2.15 (m, 1H), 2.00-1.92 (m, |
| | 1H), 1.76-1.69 (m, 2H), 1.63-1.53 (m, 2H), 1.27 (d, J = 6.4 Hz, 3H) |
| 30 | 1H NMR (400 MHz, DMSO-d6) δ = 8.27-8.22 (m, 2H), 8.18 (s, 1H), 7.70 |
| | (d, J = 8.6 Hz, 2H), 7.19-7.15 (m, 1H), 5.77 (d, J = 4.2 Hz, 1H), 5.70-5.68 |
| | (m, 2H), 5.29 (d, J = 5.2 Hz, 1H), 5.13-5.09 (m, 1H), 5.08-5.06 (m, 1H), |
| | 4.15 (q, J = 5.6 Hz, 1H), 4.03-3.95 (m, 1H), 3.91-3.86 (m, 3H), 3.44-3.37 |
| | (m, 2H), 1.92-1.85 (m, 2H), 1.63-1.54 (m, 2H), 1.25 (d, J = 6.4 Hz, 3H) |
| 31 | 1H NMR (400 MHz, DMSO-d6) δ = 8.24 (d, J = 8.6 Hz, 2H), 8.22-8.20 (m, |
| | 1H), 7.70 (d, J = 8.8 Hz, 2H), 7.33 (d, J = 7.6 Hz, 1H), 5.77 (d, J = 4.4 Hz, |
| | 1H), 5.70 (s, 2H), 5.28 (d, J = 5.4 Hz, 1H), 5.13 (q, J = 5.4 Hz, 1H), 5.08 (d, |
| | J = 5.8 Hz, 1H), 4.26-4.18 (m, 1H), 4.17-4.12 (m, 1H), 3.89 (t, J = 6.0 Hz, |
| | 1H), 3.14-3.08 (m, 2H), 2.25 (br d, J = 14.4 Hz, 3H), 2.18-2.05 (m, 3H), |
| | 1.25 (d, J = 6.4 Hz, 3H) |
| 32 | 1H NMR (400 MHz, DMSO-d6) δ = 8.25 (d, J = 8.6 Hz, 2H), 8.20 (s, 1H), |
| | 7.71 (br d, J = 8.6 Hz, 2H), 6.92 (br s, 1H), 6.94-6.89 (m, 1H), 5.78 (d, J = |
| | 4.8 Hz, 1H), 5.70 (s, 2H), 5.32 (br d, J = 5.4 Hz, 1H), 5.11 (br d, J = 5.4 Hz, |
| | 1H), 4.95-4.88 (m, 1H), 4.02 (q, J = 5.2 Hz, 1H), 3.89 (t, J = 5.8 Hz, 1H), |
| | 3.46 (br d, J = 5.8 Hz, 2H), 2.72-2.56 (m, 6H), 1.69 (br s, 4H), 1.28 (d, J = |
| | 6.4 Hz, 3H) |
| 33 | 1H NMR (400 MHz, CDCl₃-d) δ = 8.22-8.19 (m, 1H), 8.19-8.14 (m, 2H), |
| | 7.65 (br d, J = 8.6 Hz, 2H), 6.49 (br s, 1H), 5.72 (br d, J = 4.0 Hz, 2H), 4.79 |
| | (br t, J = 5.8 Hz, 1H), 4.38 (br d, J = 4.8 Hz, 1H), 4.13 (br d, J = 3.4 Hz, 1H), |
| | 3.59 (br d, J = 4.6 Hz, 2H), 2.77-2.36 (m, 12H), 2.32 (s, 3H), 1.42 (br d, J = |
| | 6.6 Hz, 3H) |
| 34 | 1H NMR (400 MHz, DMSO-d6) δ = 8.25 (d, J = 8.8 Hz, 2H), 8.18 (s, 1H), |
| | 7.71 (d, J = 8.8 Hz, 2H), 7.18 (br s, 1H), 5.71 (d, J = 4.4 Hz, 1H), 5.69 (s, |
| | 2H), 5.28 (d, J = 5.3 Hz, 1H), 5.07 (br d, J = 5.8 Hz, 2H), 4.13-4.08 (m, 1H), |
| | 3.91-3.84 (m, 1H), 3.37 (br s, 2H), 2.71 (br d, J = 11.6 Hz, 2H), 2.11 (s, 3H), |
| | 1.83-1.76 (m, 2H), 1.68-1.63 (m, 2H), 1.55-1.50 (m, 2H), 1.25 (d, J = 6.4 |
| | Hz, 4H), 1.10 (br d, J = 3.6 Hz, 2H) |
| 35 | 1H NMR (400 MHz, DMSO-d6) δ = 12.56 (br s, 1H), 9.52 (s, 1H), 8.28- |
| | 8.25 (m, 2H), 8.25-8.23 (m, 1H), 8.02 (br s, 1H), 7.73 (d, J = 8.8 Hz, 2H), |
| | 7.67 (br s, 1H), 5.90 (d, J = 4.2 Hz, 1H), 5.75 (s, 2H), 5.37 (d, J = 5.0 Hz, |
| | 1H), 5.25 (q, J = 4.8 Hz, 1H), 5.10 (d, J = 5.6 Hz, 1H), 4.18 (q, J = 5.6 Hz, |
| | 1H), 3.93 (t, J = 6.2 Hz, 1H), 1.27 (d, J = 6.4 Hz, 3H) |
| 36 | 1H NMR (400 MHz, DMSO-d6) δ = 12.70-12.58 (m, 1H), 8.25 (d, J = 8.6 |
| | Hz, 2H), 8.19 (s, 1H), 7.72 (d, J = 8.8 Hz, 2H), 7.68 (br s, 1H), 7.54-7.46 |
| | (m, 2H), 5.75-5.69 (m, 3H), 5.26 (d, J = 5.4 Hz, 1H), 5.11-5.03 (m, 2H), |
| | 4.43 (d, J = 5.6 Hz, 2H), 4.09 (q, J = 5.8 Hz, 1H), 3.86 (quin, J = 6.2 Hz, 1H), |
| | 3.32 (s, 7H), 1.24 (d, J = 6.4 Hz, 3H) |
| 37 | 1H NMR (400 MHz, DMSO-d6) δ = 8.18 (s, 1H), 7.86 (br d, J = 8.4 Hz, 2H), |
| | 7.64 (br d, J = 8.4 Hz, 2H), 7.24 (br d, J = 4.8 Hz, 1H), 5.69 (br d, J = 4.4 Hz, |
| | 1H), 5.63 (s, 2H), 5.29 (br d, J = 5.4 Hz, 1H), 5.13-5.04 (m, 2H), 4.12 (q, J = |
| | 5.2 Hz, 1H), 3.91-3.84 (m, 1H), 3.40-3.37 (m, 2H), 1.25 (br d, J = 6.4 |
| | Hz, 3H), 1.18 (br t, J = 7.2 Hz, 3H) |
| 38 | 1H NMR (400 MHz, DMSO-d6) δ = 8.20 (s, 1H), 7.54 (br d, J = 8.4 Hz, 2H), |
| | 7.42-7.05 (t, J = 74.4 Hz, 1H), 7.19 (br d, J = 8.6 Hz, 3H), 5.69 (d, J = 4.2 |
| | Hz, 1H), 5.51 (s, 2H), 5.28 (d, J = 5.4 Hz, 1H), 5.13-5.08 (m, 1H), 5.05 (d, |
| | J = 5.8 Hz, 1H), 4.15-4.10 (m, 1H), 3.92-3.82 (m, 1H), 3.37 (br d, J = 7.2 |
| | Hz, 2H), 1.25 (br d, J = 6.4 Hz, 3H), 1.17 (t, J = 7.2 Hz, 3H) |
| 39 | 1H NMR (400 MHz, DMSO-d6) δ = 8.20 (s, 1H), 7.86 (d, J = 8.4 Hz, 2H), |
| | 7.66 (d, J = 8.4 Hz, 2H), 7.47 (d, J = 2.4 Hz, 1H), 5.66 (d, J = 4.2 Hz, 1H), |
| | 5.64 (s, 2H), 5.26 (d, J = 5.2 Hz, 1H), 5.09 (q, J = 5.2 Hz, 1H), 5.04 (d, J = |
| | 5.8 Hz, 1H), 4.13 (q, J = 5.8 Hz, 1H), 3.85 (t, J = 6.2 Hz, 1H), 2.78 (br dd, J = |
| | 3.4, 6.6 Hz, 1H), 1.24 (d, J = 6.4 Hz, 3H), 0.73-0.68 (m, 2H), 0.55 (br d, |
| | J = 3.0 Hz, 2H) |
| 40 | 1H NMR (400 MHz, DMSO-d6) δ = 8.17 (s, 1H), 7.86 (d, J = 8.2 Hz, 2H), |
| | 7.64 (br d, J = 8.2 Hz, 2H), 7.05-7.01 (m, 1H), 5.75 (d, J = 4.2 Hz, 1H), 5.63 |
| | (s, 2H), 5.27 (d, J = 5.2 Hz, 1H), 5.09 (br d, J = 5.2 Hz, 1H), 5.06 (d, J = 5.8 |
| | Hz, 1H), 4.16-4.05 (m, 2H), 3.91-3.85 (m, 1H), 1.25 (br d, J = 6.4 Hz, 3H), |
| | 1.21 (d, J = 6.6 Hz, 6H) |
| 41 | 1H NMR (400 MHz, DMSO-d6) δ = 8.19 (s, 1H), 7.86 (d, J = 8.2 Hz, 2H), |
| | 7.64 (d, J = 8.2 Hz, 2H), 7.13 (t, J = 5.4 Hz, 1H), 5.73 (d, J = 4.4 Hz, 1H), |
| | 5.63 (s, 2H), 5.30 (d, J = 5.4 Hz, 1H), 5.10-5.02 (m, 2H), 4.79 (t, J = 5.6 Hz, |
| | 1H), 4.09 (q, J = 5.8 Hz, 1H), 3.92-3.85 (m, 1H), 3.58 (q, J = 6.0 Hz, 2H), |
| | 3.44-3.40 (m, 2H), 1.26 (d, J = 6.4 Hz, 3H) |
| 42 | 1H NMR (400 MHz, DMSO-d6) δ = 8.21 (s, 1H), 7.54 (d, J = 8.6 Hz, 2H), |
| | 7.23 (t, J = 74 Hz, 1H), 7.19 (d, J = 8.6 Hz, 2H), 7.08 (t, J = 5.4 Hz, 1H), 5.72 |
| | (d, J = 4.4 Hz, 1H), 5.51 (s, 2H), 5.29 (d, J = 5.4 Hz, 1H), 5.09-5.03 (m, |
| | 2H), 4.78 (t, J = 5.6 Hz, 1H), 4.10 (d, J = 5.6 Hz, 1H), 3.88 (t, J = 6.2 Hz, |
| | 1H), 3.57 (d, J = 6.0 Hz, 2H), 3.41-3.37 (m, 2H), 1.26 (d, J = 6.4 Hz, 3H) |
| 43 | 1H NMR (400 MHz, DMSO-d6) δ = 8.19 (s, 1H), 7.86 (d, J = 8.0 Hz, 2H), |
| | 7.64 (d, J = 8.0 Hz, 2H), 7.18 (t, J = 5.2 Hz, 1H), 5.71 (d, J = 4.4 Hz, 1H), |
| | 5.63 (s, 2H), 5.29 (d, J = 5.4 Hz, 1H), 5.11-5.03 (m, 2H), 4.58 (t, J = 5.4 Hz, |
| | 1H), 4.10 (q, J = 5.6 Hz, 1H), 3.87 (quin, J = 6.0 Hz, 1H), 3.48 (q, J = 6.0 Hz, |
| | 2H), 3.45-3.38 (m, 2H), 1.79-1.70 (m, 2H), 1.26 (d, J = 6.4 Hz, 3H) |
| 44 | 1H NMR (400 MHz, DMSO-d6) δ = 8.26 (s, 1H), 7.55 (d, J = 8.4 Hz, 2H), |
| | 7.42-7.05 (t, J = 74 Hz, 1H), 7.20 (d, J = 8.6 Hz, 2H), 5.73 (d, J = 4.2 Hz, |
| | 1H), 5.53 (s, 2H), 5.07 (t, J = 5.0 Hz, 1H), 4.12 (t, J = 5.6 Hz, 1H), 3.94- |
| | 3.83 (m, 2H), 3.43-3.39 (m, 2H), 3.47 (t, J = 6.2 Hz, 3H), 1.77-1.70 (m, |
| | 2H), 1.26 (d, J = 6.4 Hz, 3H) |
| 45 | 1H NMR (400 MHz, DMSO-d6) δ = 8.19 (s, 1H), 7.86 (d, J = 8.0 Hz, 2H), |
| | 7.64 (d, J = 8.2 Hz, 2H), 7.21 (br t, J = 5.4 Hz, 1H), 5.71 (d, J = 4.2 Hz, 1H), |
| | 5.63 (s, 2H), 5.30 (d, J = 5.4 Hz, 1H), 5.11-5.05 (m, 2H), 4.11 (q, J = 5.8 |
| | Hz, 1H), 3.88 (quin, J = 6.0 Hz, 1H), 3.39 (br t, J = 6.0 Hz, 4H), 3.24 (s, 3H), |
| | 1.83 (quin, J = 6.6 Hz, 2H), 1.25 (d, J = 6.4 Hz, 3H) |
| 46 | 1H NMR (400 MHz, DMSO-d6) δ = 8.21 (s, 1H), 7.54 (d, J = 8.5 Hz, 2H), |
| | 7.42-7.05 (t, J = 74 Hz, 1H), 7.21-7.14 (m, 3H), 5.70 (d, J = 4.0 Hz, 1H), |
| | 5.51 (s, 2H), 5.29 (d, J = 5.3 Hz, 1H), 5.11-5.05 (m, 2H), 4.14-4.09 (m, |
| | 1H), 3.91-3.84 (m, 1H), 3.41-3.36 (m, 4H), 3.23 (s, 3H), 1.85-1.79 (m, |
| | 2H), 1.26 (d, J = 6.3 Hz, 3H) |
| 47 | 1H NMR (400 MHz, DMSO-d6) δ = 8.26 (s, 1H), 8.23 (d, J = 3.6 Hz, 2H), |
| | 7.77 (d, J = 6.4 Hz, 1H), 7.71 (d, J = 8.6 Hz, 2H), 5.72 (d, J = 4.4 Hz, 1H), |
| | 5.70 (s, 2H), 5.33 (d, J = 5.0 Hz, 1H), 5.17 (q, J = 4.8 Hz, 1H), 5.10 (d, J = |
| | 5.6 Hz, 1H), 4.28-4.21 (m, 1H), 4.15 (q, J = 5.4 Hz, 1H), 3.90 (t, J = 6.0 Hz, |
| | 1H), 3.05-2.97 (m, 2H), 2.74 (td, J = 6.6, 17.7 Hz, 2H), 1.25 (d, J = 6.4 Hz, |
| | 3H) |
| 48 | 1H NMR (400 MHz, DMSO-d6) δ = 8.25 (d, J = 8.6 Hz, 2H), 8.18 (s, 1H), |
| | 7.71 (d, J = 8.6 Hz, 2H), 7.48 (s, 1H), 5.76 (d, J = 4.2 Hz, 1H), 5.68 (s, 2H), |
| | 5.30-5.26 (m, 1H), 5.09 (br d, J = 5.6 Hz, 2H), 5.07-5.04 (m, 1H), 4.15- |
| | 4.11 (m, 1H), 3.89-3.83 (m, 3H), 2.63-2.59 (m, 2H), 1.93-1.87 (m, 2H), |
| | 1.25 (d, J = 6.4 Hz, 3H) |
| 49 | 1H NMR (400 MHz, DMSO-d6) δ = 8.23 (s, 1H), 7.86 (d, J = 8.4 Hz, 2H), |
| | 7.77-7.74 (m, 1H), 7.64 (d, J = 8.4 Hz, 2H), 5.72 (d, J = 4.2 Hz, 1H), 5.64 |
| | (s, 2H), 5.32 (d, J = 5.0 Hz, 1H), 5.18-5.15 (m, 1H), 5.10 (d, J = 5.6 Hz, |
| | 1H), 4.27-4.21 (m, 1H), 4.17-4.13 (m, 1H), 3.92-3.88 (m, 1H), 3.03-2.96 |
| | (m, 2H), 2.78-2.69 (m, 2H), 1.25 (d, J = 6.4 Hz, 3H) |
| 50 | 1H NMR (400 MHz, DMSO-d6) δ = 8.19 (s, 1H), 7.86 (d, J = 8.2 Hz, 2H), |
| | 7.64 (d, J = 8.2 Hz, 2H), 7.44 (d, J = 6.4 Hz, 1H), 5.77 (d, J = 4.4 Hz, 1H), |
| | 5.63 (s, 2H), 5.29 (d, J = 5.2 Hz, 1H), 5.13-5.06 (m, 2H), 5.02 (d, J = 5.4 |
| | Hz, 1H), 4.43-4.28 (m, 2H), 4.14 (q, J = 5.8 Hz, 1H), 3.92-3.85 (m, 1H), |
| | 2.31-2.25 (m, 2H), 2.22-2.16 (m, 2H), 1.25 (d, J = 6.4 Hz, 3H) |
| 51 | 1H NMR (400 MHz, DMSO-d6) δ = 8.18 (s, 1H), 7.86 (d, J = 8.2 Hz, 2H), |
| | 7.64 (d, J = 8.0 Hz, 2H), 7.47 (d, J = 7.0 Hz, 1H), 5.76 (d, J = 4.4 Hz, 1H), |
| | 5.62 (s, 2H), 5.27 (d, J = 5.2 Hz, 1H), 5.11-5.08 (m, 2H), 5.05 (d, J = 5.8 |
| | Hz, 1H), 4.15-4.11 (m, 1H), 3.89-3.83 (m, 3H), 2.63-2.59 (m, 2H), 1.92- |
| | 1.86 (m, 2H), 1.25 (d, J = 6.4 Hz, 3H) |
| 52 | 1H NMR (400 MHz, DMSO-d6) δ = 8.17 (s, 1H), 7.85 (d, J = 8.2 Hz, 2H), |
| | 7.63 (d, J = 8.2 Hz, 2H), 6.98 (d, J = 7.6 Hz, 1H), 5.74 (d, J = 4.4 Hz, 1H), |
| | 5.64 (s, 2H), 5.27 (d, J = 5.0 Hz, 1H), 5.12-5.02 (m, 2H), 4.57 (d, J = 4.4 |
| | Hz, 1H), 4.12 (q, J = 5.6 Hz, 1H), 3.87 (quin, J = 6.0 Hz, 1H), 3.77-3.64 (m, |
| | 1H), 3.43-3.37 (m, 1H), 1.93 (br dd, J = 2.8, 6.2 Hz, 2H), 1.88-1.80 (m, |
| | 2H), 1.44-1.32 (m, 2H), 1.31-1.21 (m, 5H) |
| 53 | 1H NMR (400 MHz, DMSO-d6) δ = 8.19 (s, 1H), 7.54 (d, J = 8.4 Hz, 2H), |
| | 7.42-7.04 (t, J = 74, 1H), 7.19 (d, J = 8.6 Hz, 2H), 6.92 (d, J = 7.6 Hz, 1H), |
| | 5.74 (d, J = 4.2 Hz, 1H), 5.52 (s, 2H), 5.25 (d, J = 5.2 Hz, 1H), 5.11-5.01 |
| | (m, 2H), 4.55 (d, J = 4.4 Hz, 1H), 4.16-4.09 (m, 1H), 3.90-3.82 (m, 1H), |
| | 3.75-3.63 (m, 1H), 3.45-3.36 (m, 1H), 1.96-1.88 (m, 2H), 1.87-1.80 (m, |
| | 2H), 1.40-1.23 (m, 7H) |
| 54 | 1H NMR (400 MHz, DMSO-d6) δ = 8.21 (s, 1H), 7.86 (d, J = 8.2 Hz, 2H), |
| | 7.63 (d, J = 8.4 Hz, 2H), 7.32 (d, J = 7.8 Hz, 1H), 5.76 (d, J = 4.4 Hz, 1H), |
| | 5.64 (s, 2H), 5.28 (d, J = 5.4 Hz, 1H), 5.13 (q, J = 5.2 Hz, 1H), 5.08 (d, J = |
| | 5.8 Hz, 1H), 4.23-4.12 (m, 2H), 3.88 (quin, J = 6.2 Hz, 1H), 3.36 (br s, 1H), |
| | 3.30 (br s, 1H), 3.11 (br d, J = 13.2 Hz, 2H), 2.27-2.21 (m, 2H), 2.09 (br d, |
| | J = 17.4 Hz, 2H), 1.25 (d, J = 6.4 Hz, 3H) |
| 55 | 1H NMR (400 MHz, DMSO-d6) δ = 8.23 (s, 1H), 7.53 (d, J = 8.6 Hz, 2H), |
| | 7.28 (br d, J = 7.8 Hz, 1H), 7.24 (t, J = 64.8 Hz, 1H), 7.19 (d, J = 8.6 Hz, 2H), |
| | 5.75 (d, J = 4.4 Hz, 1H), 5.51 (s, 2H), 5.28 (d, J = 5.4 Hz, 1H), 5.13 (br d, J = |
| | 4.6 Hz, 1H), 5.08 (d, J = 5.6 Hz, 1H), 4.20-4.13 (m, 2H), 3.88 (t, J = 6.0 |
| | Hz, 1H), 3.29 (br s, 2H), 3.09 (br d, J = 13.0 Hz, 2H), 2.24-2.19 (m, 2H), |
| | 2.14-2.07 (m, 2H), 1.25 (d, J = 6.4 Hz, 3H) |
| 56 | 1H NMR (400 MHz, DMSO-d6) δ = 8.24 (d, J = 8.6 Hz, 2H), 8.17 (s, 1H), |
| | 7.70 (d, J = 8.6 Hz, 2H), 6.99 (br d, J = 7.0 Hz, 1H), 5.83 (d, J = 4.2 Hz, 1H), |
| | 5.69 (s, 2H), 5.24 (d, J = 5.4 Hz, 1H), 5.07-5.03 (m, 2H), 4.37 (d, J = 2.6 |
| | Hz, 1H), 4.12-4.09 (m, 1H), 3.87 (t, J = 6.0 Hz, 1H), 3.77 (br s, 2H), 1.78 |
| | (br d, J = 11.4 Hz, 2H), 1.68-1.64 (m, 4H), 1.52 (br d, J = 12.2 Hz, 2H), 1.26 |
| | (d, J = 6.4 Hz, 3H) |
| 57 | 1H NMR (400 MHz, DMSO-d6) δ = 8.25 (d, J = 8.6 Hz, 2H), 8.17 (s, 1H), |
| | 7.70 (d, J = 8.6 Hz, 2H), 7.00 (d, J = 7.6 Hz, 1H), 5.75 (d, J = 4.4 Hz, 1H), |
| | 5.70 (s, 2H), 5.27 (d, J = 5.4 Hz, 1H), 5.10-5.05 (m, 2H), 4.57 (d, J = 4.4 |
| | Hz, 1H), 4.12 (q, J = 5.6 Hz, 1H), 3.91-3.84 (m, 1H), 3.78-3.63 (m, 1H), |
| | 3.40 (br d, J = 4.6 Hz, 1H), 1.96-1.91 (m, 2H), 1.87-1.83 (m, 2H), 1.43- |
| | 1.28 (m, 4H), 1.24 (d, J = 6.4 Hz, 3H) |
| 58 | 1H NMR (400 MHz, DMSO-d6) δ = 8.25 (d, J = 8.6 Hz, 2H), 8.18 (s, 1H), |
| | 7.71 (d, J = 8.6 Hz, 2H), 7.06-6.99 (m, 1H), 5.75 (dd, J = 4.2, 9.8 Hz, 1H), |
| | 5.69 (s, 2H), 5.29 (dd, J = 3.6, 5.2 Hz, 1H), 5.11-5.05 (m, 2H), 4.57 (td, J = |
| | 5.2, 7.8 Hz, 1H), 4.16-4.09 (m, 2H), 3.88 (quin, J = 5.8 Hz, 1H), 3.46 (br d, |
| | J = 5.4 Hz, 2H), 1.81-1.72 (m, 1H), 1.70-1.63 (m, 1H), 1.25 (br d, J = 5.4 |
| | Hz, 3H), 1.20 (br d, J = 6.6 Hz, 3H) |
| 59 | 1H NMR (400 MHz, DMSO-d6) δ = 8.25 (d, J = 8.8 Hz, 2H), 8.18 (d, J = 0.6 |
| | Hz, 1H), 7.71 (br d, J = 8.6 Hz, 2H), 6.84 (br dd, J = 7.8, 12.6 Hz, 1H), 5.78 |
| | (t, J = 4.2 Hz, 1H), 5.69 (s, 2H), 5.28 (dd, J = 3.2, 5.2 Hz, 1H), 5.09 (dd, J = |
| | 3.1, 5.8 Hz, 1H), 5.02 (q, J = 4.6 Hz, 1H), 4.84-4.78 (m, 1H), 4.10 (q, J = |
| | 5.0 Hz, 1H), 4.04-3.97 (m, 1H), 3.89 (dt, J = 3.3, 5.9 Hz, 1H), 3.56-3.49 |
| | (m, 1H), 3.42-3.38 (m, 1H), 1.29-1.24 (m, 3H), 1.19 (br d, J = 6.4 Hz, 3H) |
| 60 | 1H NMR (400 MHz, DMSO-d6) δ = 8.25 (d, J = 8.8 Hz, 2H), 8.21-8.19 (m, |
| | 1H), 7.71 (d, J = 8.8 Hz, 2H), 7.42-7.37 (m, 1H), 5.72-5.70 (m, 1H), 5.70 |
| | (s, 2H), 5.31 (d, J = 5.4 Hz, 1H), 5.16-5.12 (m, 1H), 5.09 (d, J = 5.6 Hz, |
| | 1H), 4.12 (s, 1H), 3.91-3.87 (m, 1H), 3.49 (br d, J = 5.6 Hz, 2H), 3.19 (br s, |
| | 2H), 2.98 (s, 3H), 2.06-2.01 (m, 2H), 1.26 (d, J = 6.4 Hz, 3H) |
| 61 | 1H NMR (400 MHz, DMSO-d6) δ = 8.20 (s, 1H), 7.86 (d, J = 8.2 Hz, 2H), |
| | 7.64 (d, J = 8.4 Hz, 2H), 7.38 (br t, J = 5.4 Hz, 1H), 5.70 (d, J = 4.4 Hz, 1H), |
| | 5.63 (s, 2H), 5.31 (d, J = 5.4 Hz, 1H), 5.13 (q, J = 5.2 Hz, 1H), 5.08 (d, J = |
| | 5.6 Hz, 1H), 4.13 (q, J = 5.6 Hz, 1H), 3.91-3.86 (m, 1H), 3.48 (q, J = 6.6 |
| | Hz, 2H), 3.21-3.16 (m, 2H), 2.98 (s, 3H), 2.07-2.00 (m, 2H), 1.26 (d, J = |
| | 6.4 Hz, 3H) |
| 62 | 1H NMR (400 MHz, DMSO-d6) δ = 8.23 (s, 1H), 7.54 (d, J = 8.6 Hz, 2H), |
| | 7.42-7.05 (t, J = 74 Hz, 1H), 7.36 (br t, J = 5.2 Hz, 1H), 7.19 (d, J = 8.4 Hz, |
| | 2H), 5.69 (d, J = 4.4 Hz, 1H), 5.51 (s, 2H), 5.40-5.22 (m, 1H), 5.13 (br t, J = |
| | 4.8 Hz, 2H), 4.13 (t, J = 5.4 Hz, 1H), 3.93-3.84 (m, 1H), 3.49-3.43 (m, |
| | 2H), 3.20-3.15 (m, 2H), 2.97 (s, 3H), 2.05-1.98 (m, 2H), 1.26 (d, J = 6.2 |
| | Hz, 3H) |
| 63 | 1H NMR (400 MHz, DMSO-d6) δ = 9.53 (s, 1H), 8.26 (s, 1H), 7.99 (s, 1H), |
| | 7.86 (d, J = 8.4 Hz, 2H), 7.65 (d, J = 8.4 Hz, 2H), 7.56 (s, 1H), 5.89 (d, J = |
| | 4.2 Hz, 1H), 5.70 (s, 2H), 5.38 (d, J = 5.0 Hz, 1H), 5.25 (q, J = 5.0 Hz, 1H), |
| | 5.10 (d, J = 5.8 Hz, 1H), 4.18 (q, J = 5.6 Hz, 1H), 3.93 (t, J = 6.2 Hz, 1H), |
| | 3.83 (s, 3H), 1.27 (d, J = 6.4 Hz, 3H) |
| 64 | 1H NMR (400 MHz, DMSO-d6) δ = 9.49 (s, 1H), 8.29 (s, 1H), 7.97 (s, 1H), |
| | 7.57-7.54 (m, 3H), 7.25-7.17 (m, 3H), 5.88 (d, J = 4.2 Hz, 1H), 5.59 (s, |
| | 2H), 5.38 (d, J = 5.0 Hz, 1H), 5.25 (q, J = 5.0 Hz, 1H), 5.10 (d, J = 5.8 Hz, |
| | 1H), 4.18 (q, J = 5.6 Hz, 1H), 3.93 (quin, J = 6.2 Hz, 1H), 3.82 (s, 3H), 1.27 |
| | (d, J = 6.4 Hz, 3H) |
| 65 | 1H NMR (400 MHz, DMSO-d6) δ = 8.19 (s, 1H), 7.86 (d, J = 8.2 Hz, 2H), |
| | 7.65 (d, J = 8.2 Hz, 2H), 7.61 (s, 1H), 7.53 (t, J = 5.6 Hz, 1H), 7.39 (s, 1H), |
| | 5.71 (d, J = 4.4 Hz, 1H), 5.65 (s, 2H), 5.27 (d, J = 5.4 Hz, 1H), 5.09-5.05 |
| | (m, 2H), 4.38 (d, J = 5.4 Hz, 2H), 4.10 (d, J = 5.6 Hz, 1H), 3.86 (t, J = 6.2 |
| | Hz, 1H), 3.77 (s, 3H), 1.24 (d, J = 6.4 Hz, 3H) |
| 66 | 1H NMR (400 MHz, DMSO-d6) δ = 8.22 (s, 1H), 7.60 (s, 1H), 7.56-7.53 |
| | (m, 2H), 7.49-7.37 (m, 1H), 7.38 (s, 1H), 7.23 (t, J = 74 Hz, 1H), 7.21-7.18 |
| | (m, 2H), 5.71 (d, J = 4.4 Hz, 1H), 5.53 (s, 2H), 5.26 (d, J = 5.4 Hz, 1H), 5.11- |
| | 5.04 (m, 2H), 4.36 (d, J = 5.4 Hz, 2H), 4.13-4.08 (m, 1H), 3.88-3.84 (m, |
| | 1H), 3.77 (s, 3H), 1.24 (d, J = 6.4 Hz, 3H) |
| 67 | 1H NMR (400 MHz, DMSO-d6) δ = 8.26 (d, J = 8.8 Hz, 2H), 8.19 (s, 1H), |
| | 7.72 (d, J = 8.8 Hz, 2H), 7.31 (br t, J = 4.8 Hz, 1H), 5.75-5.70 (m, 3H), 5.23 |
| | (d, J = 5.4 Hz, 1H), 5.09-5.03 (m, 2H), 4.29 (d, J = 4.8 Hz, 2H), 4.08 (q, J = |
| | 5.6 Hz, 1H), 3.84 (quin, J = 6.0 Hz, 1H), 3.61 (s, 3H), 2.24 (s, 3H), 2.13 (s, |
| | 3H), 1.23 (d, J = 6.4 Hz, 3H) |
| 68 | 1H NMR (400 MHz, DMSO-d6) δ = 8.23 (s, 1H), 7.95 (s, 1H), 7.85 (d, J = |
| | 8.4 Hz, 2H), 7.62 (d, J = 8.4 Hz, 2H), 5.76 (d, J = 4.4 Hz, 1H), 5.62 (s, 2H), |
| | 5.33 (d, J = 5.4 Hz, 1H), 5.12-5.09 (m, 2H), 4.65 (d, J = 5.6 Hz, 2H), 4.12 |
| | (d, J = 5.8 Hz, 1H), 3.88 (s, 1H), 2.56 (s, 3H), 1.27 (d, J = 6.4 Hz, 3H) |
| 69 | 1H NMR (400 MHz, DMSO-d6) δ = 8.38 (s, 1H), 8.21 (s, 1H), 7.85 (d, J = |
| | 8.0 Hz, 2H), 7.69-7.57 (m, 3H), 5.80 (d, J = 4.4 Hz, 1H), 5.62 (s, 2H), 5.29 |
| | (d, J = 5.6 Hz, 1H), 5.08 (d, J = 5.8 Hz, 1H), 5.03 (q, J = 5.2 Hz, 1H), 4.57 |
| | (br d, J = 5.0 Hz, 2H), 4.09 (q, J = 5.4 Hz, 1H), 3.87 (quin, J = 6.0 Hz, 1H), |
| | 3.81 (s, 3H), 1.28 (d, J = 6.4 Hz, 3H) |
| 70 | 1H NMR (400 MHz, DMSO-d6) δ = 8.76 (s, 1H), 8.23-8.22 (m, 1H), 8.03- |
| | 7.98 (m, 3H), 7.84 (d, J = 8.4 Hz, 2H), 7.61 (br d, J = 8.2 Hz, 2H), 5.77- |
| | 5.75 (m, 1H), 5.63-5.61 (m, 2H), 5.36 (d, J = 5.4 Hz, 1H), 5.18 (q, J = 5.0 |
| | Hz, 1H), 5.11 (d, J = 5.6 Hz, 1H), 4.71 (br d, J = 4.4 Hz, 2H), 4.14-4.10 (m, |
| | 1H), 3.93-3.89 (m, 1H), 1.27 (d, J = 6.4 Hz, 3H) |
| 71 | 1H NMR (400 MHz, DMSO-d6) δ = 8.69 (s, 2H), 8.22 (s, 1H), 7.91-7.83 |
| | (m, 3H), 7.63 (d, J = 8.4 Hz, 2H), 5.72 (d, J = 4.6 Hz, 1H), 5.64 (s, 2H), 5.33 |
| | (d, J = 5.4 Hz, 1H), 5.15 (q, J = 5.2 Hz, 1H), 5.08 (d, J = 5.6 Hz, 1H), 4.55 |
| | (br d, J = 5.8 Hz, 2H), 4.11 (q, J = 5.6 Hz, 1H), 3.89 (quin, J = 6.2 Hz, 1H), |
| | 2.58 (s, 3H), 1.25 (d, J = 6.4 Hz, 3H) |
| 72 | 1H NMR (400 MHz, DMSO-d6) δ = 8.63 (s, 2H), 8.22 (s, 1H), 7.87-7.82 |
| | (m, 3H), 7.64 (d, J = 8.4 Hz, 2H), 5.71 (d, J = 4.6 Hz, 1H), 5.65 (s, 2H), 5.32 |
| | (d, J = 5.4 Hz, 1H), 5.15 (d, J = 5.0 Hz, 1H), 5.08 (d, J = 5.6 Hz, 1H), 4.52 |
| | (d, J = 5.0 Hz, 2H), 4.11 (d, J = 5.6 Hz, 1H), 3.90-3.87 (m, 4H), 1.25 (d, J = |
| | 6.4 Hz, 3H) |
| 73 | 1H NMR (400 MHz, DMSO-d6) δ = 8.19 (s, 1H), 7.86 (d, J = 8.2 Hz, 2H), |
| | 7.63 (d, J = 8.0 Hz, 2H), 7.19-7.15 (m, 1H), 5.76 (d, J = 4.0 Hz, 1H), 5.63 |
| | (s, 2H), 5.30 (d, J = 5.4 Hz, 1H), 5.13-5.10 (m, 1H), 5.09-5.07 (m, 1H), |
| | 4.17-4.12 (m, 1H), 4.02-3.96 (m, 1H), 3.91-3.88 (m, 2H), 3.87-3.86 (m, |
| | 1H), 3.40 (s, 2H), 1.91-1.85 (m, 2H), 1.63-1.55 (m, 2H), 1.25 (d, J = 6.4 |
| | Hz, 3H) |
| 74 | 1H NMR (400 MHz, DMSO-d6) δ = 8.87 (s, 1H), 8.19 (s, 1H), 8.14-8.10 |
| | (m, 1H), 8.08-8.04 (m, 1H), 7.11 (br d, J = 6.4 Hz, 1H), 5.78 (br d, J = 3.8 |
| | Hz, 1H), 5.67 (s, 2H), 5.27 (br d, J = 5.0 Hz, 1H), 5.07 (br d, J = 5.6 Hz, 2H), |
| | 4.22-4.16 (m, 1H), 4.16-4.11 (m, 1H), 3.90-3.85 (m, 1H), 1.97-1.91 (m, |
| | 2H), 1.69 (br s, 2H), 1.54 (br s, 4H), 1.24 (br d, J = 5.8 Hz, 3H) |
| 76 | 1H NMR (400 MHz, DMSO-d6) δ = 8.87 (d, J = 1.4 Hz, 1H), 8.20 (s, 1H), |
| | 8.13-8.10 (m, 1H), 8.08-8.04 (m, 1H), 7.17 (br d, J = 7.6 Hz, 1H), 5.76 (d, |
| | J = 4.2 Hz, 1H), 5.67 (s, 2H), 5.28 (d, J = 5.4 Hz, 1H), 5.10 (br d, J = 4.6 Hz, |
| | 1H), 5.07 (d, J = 5.8 Hz, 1H), 4.15 (d, J = 5.8 Hz, 1H), 4.00-3.96 (m, 1H), |
| | 3.91-3.88 (m, 2H), 3.88-3.86 (m, 1H), 3.40 (br d, J = 1.8 Hz, 2H), 1.90- |
| | 1.85 (m, 2H), 1.59 (br dd, J = 6.0, 11.7 Hz, 2H), 1.25 (d, J = 6.4 Hz, 3H) |
| 77 | 1H NMR (400 MHz, DMSO-d6) δ = 8.21 (s, 1H), 7.86 (d, J = 8.0 Hz, 2H), |
| | 7.64 (br d, J = 8.2 Hz, 2H), 7.38 (br d, J = 5.8 Hz, 1H), 5.80 (br d, J = 4.2 Hz, |
| | 1H), 5.71-5.56 (m, 2H), 5.28 (d, J = 5.2 Hz, 1H), 5.19-4.96 (m, 2H), 4.44 |
| | (br s, 1H), 4.18-4.11 (m, 1H), 3.91-3.83 (m, 3H), 3.74-3.69 (m, 1H), 3.68- |
| | 3.63 (m, 1H), 2.25-2.16 (m, 1H), 2.01-1.93 (m, 1H), 1.25 (d, J = 6.4 Hz, |
| | 3H) |
| 78 | 1H NMR (400 MHz, DMSO-d6) δ = 8.21 (s, 1H), 7.86 (d, J = 8.4 Hz, 2H), |
| | 7.64 (d, J = 8.4 Hz, 2H), 7.36 (d, J = 6.4 Hz, 1H), 5.80 (d, J = 4.4 Hz, 1H), |
| | 5.64 (s, 2H), 5.26 (d, J = 5.4 Hz, 1H), 5.11-5.08 (m, 1H), 5.07 (d, J = 5.8 |
| | Hz, 1H), 4.47-4.41 (m, 1H), 4.13 (q, J = 5.8 Hz, 1H), 3.89-3.85 (m, 3H), |
| | 3.74-3.69 (m, 1H), 3.66 (dd, J = 3.8, 9.0 Hz, 1H), 2.23-2.17 (m, 1H), 1.99- |
| | 1.93 (m, 1H), 1.25 (d, J = 6.4 Hz, 3H) |
| 79 | 1H NMR (400 MHz, DMSO-d6) δ = 8.22 (s, 1H), 7.57-7.50 (m, 2H), 7.42 |
| | (s, 1H), 7.23 (s, 1H), 7.20 (d, J = 8.6 Hz, 2H), 7.08 (dd, J = 3.0, 7.5 Hz, 1H), |
| | 7.05 (s, 1H), 5.74 (d, J = 4.2 Hz, 1H), 5.51 (s, 2H), 5.27 (dd, J = 5.4, 6.8 Hz, |
| | 1H), 5.13-5.04 (m, 2H), 4.36-4.29 (m, 1H), 4.14 (q, J = 5.6 Hz, 1H), 4.05- |
| | 3.98 (m, 1H), 3.90-3.78 (m, 2H), 3.19-3.09 (m, 1H), 2.71-2.64 (m, 1H), |
| | 2.01-1.90 (m, 5H), 1.52-1.43 (m, 1H), 1.39-1.31 (m, 1H), 1.25 (d, J = 6.4 |
| | Hz, 3H) |
| 80 | 1H NMR (400 MHz, DMSO-d6) δ = 8.19 (s, 1H), 7.86 (d, J = 8.2 Hz, 2H), |
| | 7.64 (d, J = 8.2 Hz, 2H), 7.14 (br dd, J = 2.6, 7.6 Hz, 1H), 5.75 (d, J = 4.4 Hz, |
| | 1H), 5.63 (s, 2H), 5.30-5.27 (m, 1H), 5.11-5.06 (m, 2H), 4.34 (br d, J = |
| | 12.0 Hz, 1H), 4.14 (q, J = 5.6 Hz, 1H), 4.05-3.99 (m, 1H), 3.88 (t, J = 6.2 |
| | Hz, 1H), 3.85 (br s, 1H), 3.15 (br t, J = 11.8 Hz, 1H), 2.68 (br t, J = 11.6 Hz, |
| | 1H), 2.01 (s, 3H), 1.98-1.88 (m, 2H), 1.54-1.45 (m, 1H), 1.40-1.32 (m, |
| | 1H), 1.25 (d, J = 6.4 Hz, 3H) |
| 81 | 1H NMR (400 MHz, DMSO-d6) δ = 8.21 (s, 1H), 7.86 (d, J = 8.4 Hz, 2H), |
| | 7.64 (d, J = 8.4 Hz, 2H), 7.45 (d, J = 7.0 Hz, 1H), 5.75 (d, J = 4.4 Hz, 1H), |
| | 5.64 (s, 2H), 5.31 (d, J = 5.4 Hz, 1H), 5.13 (q, J = 5.2 Hz, 1H), 5.09 (d, J = |
| | 5.8 Hz, 1H), 4.46-4.39 (m, 1H), 4.14 (q, J = 5.6 Hz, 1H), 3.89 (t, J = 6.2 Hz, |
| | 1H), 2.58 (br, dd, J = 8.2, 11.4 Hz, 1H), 2.28-2.16 (m, 4H), 1.86-1.80 (m, |
| | 1H), 1.25 (d, J = 6.4 Hz, 3H) |
| 82 | 1H NMR (400 MHz, DMSO-d6) δ = 8.26 (s, 1H), 7.90 (d, J = 8.2 Hz, 2H), |
| | 7.68 (d, J = 8.2 Hz, 2H), 7.49 (br d, J = 7.4 Hz, 1H), 5.79 (d, J = 4.2 Hz, 1H), |
| | 5.68 (s, 2H), 5.35 (d, J = 5.4 Hz, 1H), 5.19-5.13 (m, 2H), 4.51-4.44 (m, |
| | 1H), 4.19 (q, J = 5.4 Hz, 1H), 3.96-3.91 (m, 1H), 2.31-2.17 (m, 5H), 1.89 |
| | (br dd, J = 8.6, 11.6 Hz, 1H), 1.30 (d, J = 6.4 Hz, 3H) |
| 83 | 1H NMR (400 MHz, DMSO-d6) δ = 8.26 (d, J = 8.8 Hz, 2H), 8.22 (s, 1H), |
| | 7.72 (d, J = 8.8 Hz, 2H), 7.49-7.46 (m, 1H), 5.76 (d, J = 4.4 Hz, 1H), 5.71 |
| | (s, 2H), 5.32 (d, J = 5.2 Hz, 1H), 5.16-5.13 (m, 1H), 5.10 (d, J = 5.8 Hz, |
| | 1H), 4.47-4.40 (m, 1H), 4.15 (s, 1H), 3.92-3.88 (m, 1H), 2.63-2.55 (m, |
| | 1H), 2.34-2.26 (m, 1H), 2.26-2.20 (m, 2H), 2.17-2.09 (m, 1H), 1.88-1.81 |
| | (m, 1H), 1.26 (d, J = 6.2 Hz, 3H) |
| 84 | 1H NMR (400 MHz, DMSO-d6) δ = 8.25 (d, J = 8.6 Hz, 2H), 8.21 (s, 1H), |
| | 7.71 (d, J = 8.6 Hz, 2H), 7.46 (d, J = 7.1 Hz, 1H), 5.75 (d, J = 4.4 Hz, 1H), |
| | 5.70 (s, 2H), 5.31 (d, J = 5.4 Hz, 1H), 5.15-5.08 (m, 2H), 4.48-4.39 (m, |
| | 1H), 4.14 (q, J = 5.6 Hz, 1H), 3.89 (t, J = 6.0 Hz, 1H), 2.65 (br d, J = 15.8 |
| | Hz, 1H), 2.30-2.11 (m, 4H), 1.85 (br dd, J = 8.6, 11.8 Hz, 1H), 1.25 (d, J = |
| | 6.4 Hz, 3H) |
| 85 | 1H NMR (400 MHz, DMSO-d6) δ = 8.79 (d, J = 2.0 Hz, 1H), 8.35-8.33 (m, |
| | 1H), 8.31-8.28 (m, 1H), 8.20 (s, 1H), 7.11 (d, J = 7.0 Hz, 1H), 5.78 (d, J = |
| | 4.4 Hz, 1H), 5.74 (s, 2H), 5.26 (d, J = 5.4 Hz, 1H), 5.09-5.05 (m, 2H), 4.21 |
| | (br d, J = 6.4 Hz, 1H), 4.13 (d, J = 5.6 Hz, 1H), 3.88 (t, J = 6.2 Hz, 1H), 1.99- |
| | 1.94 (m, 2H), 1.71-1.67 (m, 2H), 1.58-1.53 (m, 4H), 1.25 (d, J = 6.4 Hz, |
| | 3H) |
| 86 | 1H NMR (400 MHz, DMSO-d6) δ = 8.18 (s, 1H), 8.01 (d, J = 8.4 Hz, 1H), |
| | 7.57 (s, 1H), 7.51 (d, J = 8.4 Hz, 1H), 7.10 (d, J = 6.8 Hz, 1H), 5.78 (d, J = |
| | 4.2 Hz, 1H), 5.61 (s, 2H), 5.27 (d, J = 5.2 Hz, 1H), 5.09-5.06 (m, 2H), 4.21 |
| | (br d, J = 6.6 Hz, 1H), 4.13 (d, J = 5.6 Hz, 1H), 3.87 (t, J = 6.2 Hz, 1H), 2.53 |
| | (s, 3H), 1.95 (br dd, J = 3.6, 6.4 Hz, 2H), 1.69 (br s, 2H), 1.58-1.52 (m, 4H), |
| | 1.25 (d, J = 6.4 Hz, 3H) |
| 87 | 1H NMR (400 MHz, DMSO-d6) δ = 8.52 (d, J = 2.4 Hz, 1H), 8.22 (s, 1H), |
| | 8.15 (dd, J = 2.4, 8.4 Hz, 1H), 7.33 (d, J = 8.4 Hz, 1H), 7.08 (d, J = 6.8 Hz, |
| | 1H), 5.77 (d, J = 4.2 Hz, 1H), 5.58 (s, 2H), 5.26 (d, J = 5.2 z, 1H), 5.09-5.05 |
| | (m, 2H), 4.21-4.11 (m, 2H), 3.87 (t, J = 6.2 Hz, 1H), 1.98-1.91 (m, 2H), |
| | 1.71-1.64 (m, 2H), 1.60-1.52 (m, 4H), 1.25 (d, J = 6.4 Hz, 3H) |
| 88 | 1H NMR (400 MHz, DMSO-d6) δ = 8.20 (s, 1H), 7.61 (d, J = 8.6 Hz, 2H), |
| | 7.39 (br d, J = 8.0 Hz, 2H), 7.05 (br d, J = 6.8 Hz, 1H), 5.77 (d, J = 4.2 Hz, |
| | 1H), 5.55 (s, 2H), 5.26 (d, J = 5.4 Hz, 1H), 5.10-5.02 (m, 2H), 4.22-4.11 |
| | (m, 2H), 3.87 (quin, J = 5.8 Hz, 1H), 1.97-1.93 (m, 2H), 1.68 (br s, 2H), |
| | 1.54 (br s, 4H), 1.25 (br d, J = 6.4 Hz, 3H) |
| 89 | 1H NMR (400 MHz, DMSO-d6) δ = 8.18 (s, 1H), 7.95 (t, J = 7.4 Hz, 1H), |
| | 7.61 (d, J = 10.4 Hz, 1H), 7.48 (d, J = 7.6 Hz, 1H), 7.11 (d, J = 7.0 Hz, 1H), |
| | 5.78 (d, J = 4.2 Hz, 1H), 5.63 (s, 2H), 5.27 (d, J = 5.4 Hz, 1H), 5.10-5.05 |
| | (m, 2H), 4.25-4.19 (m, 1H), 4.13 (q, J = 5.8 Hz, 1H), 3.88 (t, J = 6.2 Hz, |
| | 1H), 2.02-1.92 (m, 2H), 1.72-1.65 (m, 2H), 1.60-1.52 (m, 4H), 1.25 (d, J = |
| | 6.4 Hz, 3H) |
| 90 | 1H NMR (400 MHz, DMSO-d6) δ = 8.20 (s, 1H), 7.36 (d, J = 7.8 Hz, 2H), |
| | 7.19 (d, J = 7.8 Hz, 2H), 7.01 (d, J = 6.8 Hz, 1H), 5.77 (d, J = 4.4 Hz, 1H), |
| | 5.47 (s, 2H), 5.25 (d, J = 5.4 Hz, 1H), 5.09-5.03 (m, 2H), 4.19-4.11 (m, |
| | 2H), 3.90-3.84 (m, 1H), 2.30 (s, 3H), 1.93 (br s, 2H), 1.70-1.66 (m, 2H), |
| | 1.53 (br s, 4H), 1.25 (d, J = 6.4 Hz, 3H) |
| 91 | 1H NMR (400 MHz, DMSO-d6) δ = 8.20 (s, 1H), 7.52-7.49 (m, 2H), 7.48- |
| | 7.44 (m, 2H), 7.05 (d, J = 6.8 Hz, 1H), 5.78 (d, J = 4.2 Hz, 1H), 5.53 (s, |
| | 2H), 5.26 (d, J = 5.4 Hz, 1H), 5.08 (br d, J = 4.6 Hz, 1H), 5.05 (d, J = 5.8 Hz, |
| | 1H), 4.23-4.18 (m, 1H), 4.14 (br d, J = 5.6 Hz, 1H), 3.88 (t, J = 6.2 Hz, 1H), |
| | 1.98-1.92 (m, 2H), 1.69 (br s, 2H), 1.58-1.53 (m, 4H), 1.26 (d, J = 6.4 Hz, |
| | 3H) |
| 92 | 1H NMR (400 MHz, DMSO-d6) δ = 8.20 (s, 1H), 7.52-7.49 (m, 2H), 7.48- |
| | 7.44 (m, 2H), 7.05 (d, J = 6.8 Hz, 1H), 5.78 (d, J = 4.2 Hz, 1H), 5.53 (s, |
| | 2H), 5.26 (d, J = 5.4 Hz, 1H), 5.08 (br d, J = 4.6 Hz, 1H), 5.05 (d, J = 5.8 Hz, |
| | 1H), 4.23-4.18 (m, 1H), 4.14 (br d, J = 5.6 Hz, 1H), 3.88 (t, J = 6.2 Hz, 1H), |
| | 1.98-1.92 (m, 2H), 1.69 (br s, 2H), 1.58-1.53 (m, 4H), 1.26 (d, J = 6.4 Hz, |
| | 3H) |
| 93 | 1H NMR (400 MHz, DMSO-d6) δ = 8.90 (d, J = 1.6 Hz, 1H), 8.20 (s, 1H), |
| | 8.19-8.15 (m, 1H), 7.94 (d, J = 8.2 Hz, 1H), 7.10 (d, J = 6.8 Hz, 1H), 5.78 (d, |
| | J = 4.2 Hz, 1H), 5.68 (s, 2H), 5.27 (d, J = 5.4 Hz, 1H), 5.10-5.05 (m, 2H), |
| | 4.23-4.11 (m, 2H), 3.87 (quin, J = 6.2 Hz, 1H), 1.99-1.91 (m, 2H), 1.72- |
| | 1.66 (m, 2H), 1.59-1.52 (m, 4H), 1.25 (d, J = 6.4 Hz, 3H) |
| 94 | 1H NMR (400 MHz, DMSO-d6) δ = 8.19 (s, 1H), 7.59 (s, 4H), 7.06 (d, J = |
| | 7.0 Hz, 1H), 7.04 (t, J = 56 Hz 1H), 5.78 (d, J = 4.4 Hz, 1H), 5.59 (s, 2H), |
| | 5.27 (d, J = 5.4 Hz, 1H), 5.10-5.05 (m, 2H), 4.24-4.17 (m, 1H), 4.14 (q, J = |
| | 5.8 Hz, 1H), 3.87 (t, J = 6.2 Hz, 1H), 1.98-1.90 (m, 2H), 1.71-1.64 (m, |
| | 2H), 1.60-1.52 (m, 4H), 1.25 (d, J = 6.4 Hz, 3H) |
| 95 | 1H NMR (400 MHz, DMSO-d6) δ = 8.81 (s, 1H), 8.20 (s, 1H), 8.09 (br d, J = |
| | 7.6 Hz, 1H), 7.73 (d, J = 8.0 Hz, 1H), 7.10 (s, 1H), 7.07 (br d, J = 6.8 Hz, |
| | 1H), 6.97 (s, 1H), 6.83 (s, 1H), 5.78 (d, J = 4.0 Hz, 1H), 5.64 (s, 2H), 5.25 (d, |
| | J = 5.4 Hz, 1H), 5.08-5.04 (m, 2H), 4.19-4.11 (m, 2H), 3.89-3.85 (m, 1H), |
| | 1.95 (br d, J = 2.4 Hz, 2H), 1.69 (br s, 2H), 1.54 (br s, 4H), 1.25 (d, J = 6.4 |
| | Hz, 3H) |
| 96 | 1H NMR (400 MHz, DMSO-d6) δ = 8.20 (s, 1H), 7.52-7.49 (m, 2H), 7.47- |
| | 7.44 (m, 2H), 7.03-6.95 (m, 1H), 5.77-5.73 (m, 1H), 5.53 (s, 2H), 5.32- |
| | 5.28 (m, 1H), 5.10-5.06 (m, 2H), 4.60-4.53 (m, 1H), 4.15-4.10 (m, 2H), |
| | 3.92-3.85 (m, 1H), 3.49-3.44 (m, 2H), 1.79-1.72 (m, 1H), 1.69-1.62 (m, |
| | 1H), 1.27-1.25 (m, 3H), 1.20 (d, J = 5.8 Hz, 3H) |
| 97 | 1H NMR (400 MHz, DMSO-d6) δ = 8.19 (s, 1H), 7.51-7.48 (m, 2H), 7.47- |
| | 7.43 (m, 2H), 6.97 (dd, J = 8.4, 10.4 Hz, 1H), 5.74 (dd, J = 4.4, 9.6 Hz, 1H), |
| | 5.52 (s, 2H), 5.28 (dd, J = 3.2, 5.3 Hz, 1H), 5.09-5.05 (m, 2H), 4.55 (td, J = |
| | 5.2, 7.3 Hz, 1H), 4.12 (dq, J = 2.4, 5.6 Hz, 2H), 3.87 (t, J = 6.0 Hz, 1H), 3.48- |
| | 3.44 (m, 2H), 1.78-1.72 (m, 1H), 1.68-1.61 (m, 1H), 1.26 (dd, J = 1.4, 6.3 |
| | Hz, 3H), 1.19 (dd, J = 1.0, 6.5 Hz, 3H) |
| 98 | 1H NMR (400 MHz, DMSO-d6) δ = 8.21 (s, 1H), 7.56-7.51 (m, 2H), 7.24- |
| | 7.19 (m, 2H), 7.00-6.93 (m, 1H), 5.74 (dd, J = 4.4, 9.8 Hz, 1H), 5.50 (s, |
| | 2H), 5.29 (dd, J = 3.4, 5.4 Hz, 1H), 5.09-5.05 (m, 2H), 4.58-4.52 (m, 1H), |
| | 4.14-4.08 (m, 2H), 3.87 (t, J = 5.8 Hz, 1H), 3.45 (br dd, J = 2.4, 5.8 Hz, 2H), |
| | 1.77-1.65 (m, 2H), 1.25 (dd, J = 1.6, 6.4 Hz, 3H), 1.18 (d, J = 6.4 Hz, 3H) |
| 99 | 1H NMR (400 MHz, DMSO-d6) δ = 8.20 (s, 1H), 7.61 (d, J = 8.4 Hz, 2H), |
| | 7.39 (d, J = 8.2 Hz, 2H), 7.01-6.95 (m, 1H), 5.74 (dd, J = 4.4, 9.8 Hz, 1H), |
| | 5.55 (s, 2H), 5.29 (dd, J = 3.4, 5.2 Hz, 1H), 5.10-5.06 (m, 2H), 4.56 (td, J = |
| | 5.2, 7.3 Hz, 1H), 4.16-4.09 (m, 2H), 3.88 (t, J = 6.2 Hz, 1H), 3.49-3.44 (m, |
| | 2H), 1.78-1.71 (m, 1H), 1.69-1.61 (m, 1H), 1.26 (dd, J = 1.4, 6.3 Hz, 3H), |
| | 1.19 (d, J = 6.4 Hz, 3H) |
| 100 | 1H NMR (400 MHz, DMSO-d6) δ = 8.20 (s, 1H), 7.77-7.74 (m, 2H), 7.69- |
| | 7.66 (m, 2H), 6.99 (d, J = 7.6 Hz, 1H), 5.77 (d, J = 4.6 Hz, 1H), 5.64 (s, |
| | 2H), 5.30-5.27 (m, 1H), 5.09-5.05 (m, 1H), 4.96 (br d, J = 5.4 Hz, 1H), |
| | 4.83-4.79 (m, 1H), 4.22 (br d, J = 7.0 Hz, 1H), 4.13 (br d, J = 3.6 Hz, 1H), |
| | 4.08-4.04 (m, 1H), 3.89-3.85 (m, 1H), 2.16 (br d, J = 6.6 Hz, 1H), 1.95 (br |
| | s, 1H), 1.75-1.69 (m, 2H), 1.64-1.60 (m, 1H), 1.55 (br d, J = 13.6 Hz, 1H), |
| | 1.27 (d, J = 6.4 Hz, 3H) |
| 101 | 1H NMR (400 MHz, DMSO-d6) δ = 8.20 (s, 1H), 7.52-7.49 (m, 2H), 7.47- |
| | 7.44 (m, 2H), 6.96 (d, J = 7.6 Hz, 1H), 5.77 (d, J = 4.6 Hz, 1H), 5.52 (s, |
| | 2H), 5.29-5.27 (m, 1H), 5.08-5.05 (m, 1H), 4.97-4.91 (m, 1H), 4.80 (s, |
| | 1H), 4.24-4.17 (m, 1H), 4.15-4.10 (m, 1H), 4.08-4.03 (m, 1H), 3.89-3.84 |
| | (m, 1H), 2.19-2.13 (m, 1H), 1.98-1.92 (m, 1H), 1.74-1.68 (m, 2H), 1.64- |
| | 1.59 (m, 1H), 1.55 (br dd, J = 6.2, 12.9 Hz, 1H), 1.27 (d, J = 6.4 Hz, 3H) |
| 102 | 1H NMR (400 MHz, DMSO-d6) δ = 8.21 (s, 1H), 7.56-7.52 (m, 2H), 7.22 |
| | (t, J = 8.4 Hz, 2H), 6.94 (br d, J = 7.6 Hz, 1H), 5.76 (d, J = 4.6 Hz, 1H), 5.50 |
| | (s, 2H), 5.28 (br d, J = 5.4 Hz, 1H), 5.08-5.05 (m, 1H), 4.94 (br s, 1H), 4.81- |
| | 4.78 (m, 1H), 4.21-4.17 (m, 1H), 4.12 (br s, 1H), 4.07-4.04 (m, 1H), 3.86 |
| | (br d, J = 4.2 Hz, 1H), 2.15 (br s, 1H), 1.94 (br s, 1H), 1.71 (br t, J = 6.6 Hz, |
| | 2H), 1.61 (br s, 1H), 1.55 (br s, 1H), 1.27 (d, J = 6.4 Hz, 3H) |
| 103 | 1H NMR (400 MHz, DMSO-d6) δ = 8.19 (s, 1H), 8.00 (d, J = 8.4 Hz, 1H), |
| | 7.57 (s, 1H), 7.50 (d, J = 8.4 Hz, 1H), 7.17 (d, J = 7.6 Hz, 1H), 5.76 (d, J = |
| | 4.2 Hz, 1H), 5.60 (s, 2H), 5.30 (d, J = 5.34 Hz, 1H), 5.11 (br d, J = 4.8 Hz, |
| | 1H), 5.08 (d, J = 5.8 Hz, 1H), 4.15 (q, J = 5.6 Hz, 1H), 3.99 (dt, J = 6.6, 10.8 |
| | Hz, 1H), 3.92-3.86 (m, 3H), 3.44-3.36 (m, 2H), 2.52 (s, 3H), 1.92-1.84 |
| | (m, 2H), 1.64-1.54 (m, 2H), 1.25 (d, J = 6.4 Hz, 3H) |
| 104 | 1H NMR (400 MHz, DMSO-d6) δ = 8.20 (s, 1H), 7.88-7.83 (m, 2H), 7.64 |
| | (d, J = 8.6 Hz, 2H), 7.42 (br t, J = 5.6 Hz, 1H), 5.70 (d, J = 4.2 Hz, 1H), 5.63 |
| | (s, 2H), 5.30 (d, J = 5.2 Hz, 1H), 5.15-5.11 (m, 1H), 5.08 (d, J = 5.6 Hz, |
| | 1H), 4.13 (q, J = 5.4 Hz, 1H), 3.94-3.85 (m, 1H), 3.48 (br t, J = 6.2 Hz, 2H), |
| | 2.67-2.61 (m, 2H), 2.60 (br d, J = 9.4 Hz, 1H), 2.37 (br dd, J = 6.2, 14.3 Hz, |
| | 2H), 1.25 (d, J = 6.4 Hz, 3H) |
| 105 | 1H NMR (400 MHz, DMSO-d6) δ = 8.16 (s, 1H), 7.82 (d, J = 8.2 Hz, 2H), |
| | 7.60 (d, J = 8.2 Hz, 2H), 7.30 (br s, 1H), 5.69 (d, J = 4.4 Hz, 1H), 5.60 (s, |
| | 2H), 5.26 (d, J = 5.4 Hz, 1H), 5.08 (q, J = 4.8 Hz, 1H), 5.04 (d, J = 5.6 Hz, |
| | 1H), 4.08 (q, J = 5.6 Hz, 1H), 3.85 (t, J = 6.2 Hz, 1H), 3.33 (br s, 2H), 2.18- |
| | 2.03 (m, 4H), 1.89-1.83 (m, 2H), 1.52-1.46 (m, 1H), 1.22 (d, J = 6.4 Hz, |
| | 3H) |
| 106 | 1H NMR (400 MHz, DMSO-d6) δ = 8.18 (s, 1H), 7.86 (d, J = 8.2 Hz, 2H), |
| | 7.63 (d, J = 8.2 Hz, 2H), 7.33 (s, 1H), 5.74 (d, J = 4.4 Hz, 1H), 5.63 (s, 2H), |
| | 5.30 (d, J = 5.4 Hz, 1H), 5.12 (br d, J = 4.8 Hz, 1H), 5.08 (d, J = 5.6 Hz, 1H), |
| | 4.12 (d, J = 5.6 Hz, 1H), 3.89 (br d, J = 6.0 Hz, 1H), 3.26 (br t, J = 5.8 Hz, |
| | 2H), 2.01 (br d, J = 9.0 Hz, 2H), 1.83-1.77 (m, 5H), 1.25 (d, J = 6.4 Hz, 3H), |
| | 1.22-1.17 (m, 2H) |
| 107 | 1H NMR (400 MHz, DMSO-d6) δ = 8.12 (s, 1H), 7.85 (d, J = 8.4 Hz, 2H), |
| | 7.65 (d, J = 8.4 Hz, 2H), 5.71 (d, J = 4.4 Hz, 1H), 5.61 (s, 2H), 5.12 (d, J = |
| | 5.6 Hz, 1H), 4.92 (d, J = 6.0 Hz, 1H), 4.88-4.85 (m, 1H), 4.76 (d, J = 3.4 |
| | Hz, 1H), 4.19-4.14 (m, 1H), 4.06 (dd, J = 3.8, 5.8 Hz, 1H), 3.79-3.75 (m, |
| | 2H), 3.50-3.42 (m, 2H), 3.41-3.38 (m, 1H), 3.13-3.09 (m, 1H), 2.01-1.96 |
| | (m, 1H), 1.21 (d, J = 6.4 Hz, 3H) |
| 108 | 1H NMR (400 MHz, DMSO-d6) δ = 8.19 (s, 1H), 7.86 (d, J = 8.2 Hz, 2H), |
| | 7.64 (d, J = 8.2 Hz, 2H), 7.36 (br t, J = 5.2 Hz, 1H), 5.72 (d, J = 4.4 Hz, 1H), |
| | 5.63 (s, 2H), 5.30 (d, J = 5.2 Hz, 1H), 5.12-5.07 (m, 2H), 4.13-4.10 (m, |
| | 1H), 3.89 (quin, J = 6.0 Hz, 2H), 3.75 (br d, J = 6.0 Hz, 1H), 3.67 (d, J = |
| | 7.2 Hz, 1H), 3.61 (d, J = 7.4 Hz, 1H), 3.50 (dd, J = 5.2 8.4 Hz, 2H), 2.62 (br |
| | s, 1H), 1.96-1.92 (m, 1H), 1.62-1.58 (m, 1H), 1.26 (d, J = 6.4 Hz, 3H) |
| 109 | 1H NMR (400 MHz, DMSO-d6) δ = 8.17 (s, 1H), 7.85 (d, J = 8.1 Hz, 2H), |
| | 7.63 (d, J = 8.2 Hz, 2H), 7.26-7.22 (m, 1H), 5.73 (d, J = 4.6 Hz, 1H), 5.62 |
| | (s, 2H), 5.27 (d, J = 5.4 Hz, 1H), 5.12-5.08 (m, 1H), 5.05 (d, J = 5.8 Hz, |
| | 1H), 4.12-4.08 (m, 1H), 3.88 (br d, J = 6.0 Hz, 1H), 3.86-3.81 (m, 2H), |
| | 3.26-3.22 (m, 4H), 1.91 (dt, J = 3.8, 7.6 Hz, 1H), 1.61 (br d, J = 12.4 Hz, |
| | 2H), 1.24 (d, J = 6.4 Hz, 3H), 1.18 (br dd, J = 3.8, 12.4 Hz, 2H) |
| 110 | 1H NMR (400 MHz, DMSO-d6) δ = 8.21 (s, 1H), 7.51-7.48 (m, 2H), 7.47- |
| | 7.43 (m, 2H), 7.34 (s, 1H), 5.72 (d, J = 4.4 Hz, 1H), 5.52 (s, 2H), 5.30 (d, J = |
| | 5.2 Hz, 1H), 5.11 (br d, J = 5.4 Hz, 1H), 5.07 (d, J = 5.8 Hz, 1H), 4.12 (d, |
| | J = 5.6 Hz, 1H), 3.88 (t, J = 6.0 Hz, 1H), 3.74 (br d, J = 5.8 Hz, 1H), 3.67 (dd, |
| | J = 7.0, 8.5 Hz, 1H), 3.61 (d, J = 7.0 Hz, 1H), 3.49 (dd, J = 5.2, 8.5 Hz, 1H), |
| | 3.30 (br s, 1H), 2.62-2.57 (m, 2H), 1.97-1.91 (m, 1H), 1.63-1.57 (m, 1H), |
| | 1.26 (d, J = 6.4 Hz, 3H) |
| 111 | 1H NMR (400 MHz, DMSO-d6) δ = 8.20 (s, 1H), 7.52-7.49 (m, 2H), 7.46- |
| | 7.43 (m, 2H), 7.23 (s, 1H), 5.73 (d, J = 4.4 Hz, 1H), 5.52 (s, 2H), 5.29 (d, J = |
| | 5.4 Hz, 1H), 5.11 (br d, J = 4.8 Hz, 1H), 5.07 (d, J = 5.8 Hz, 1H), 4.12 (d, |
| | J = 5.6 Hz, 1H), 3.89 (d, J = 6.0 Hz, 1H), 3.84 (br dd, J = 2.4, 11.4 Hz, 2H), |
| | 3.26-3.21 (m, 4H), 1.94-1.87 (m, 1H), 1.63-1.58 (m, 2H), 1.25 (d, J = 6.2 |
| | Hz, 3H), 1.21-1.15 (m, 2H) |
| 112 | 1H NMR (400 MHz, DMSO-d6) δ = 8.21 (d, J = 1.4 Hz, 1H), 7.55-7.53 (m, |
| | 2H), 7.41 (d, J = 1.4 Hz, 1H), 7.32 (br t, J = 5.2 Hz, 1H), 7.23 (d, J = 1.6 Hz, |
| | 1H), 7.19 (d, J = 8.4 Hz, 2H), 7.04 (d, J = 1.6 Hz, 1H), 5.71 (d, J = 4.2 Hz, |
| | 1H), 5.51 (s, 2H), 5.29 (d, J = 5.4 Hz, 1H), 5.09 (br d, J = 8.8 Hz, 2H), 4.13- |
| | 4.10 (m, 1H), 3.88 (br t, J = 5.8 Hz, 1H), 3.77-3.70 (m, 2H), 3.67 (t, J = |
| | 7.8 Hz, 2H), 3.60 (br d, J = 7.6 Hz, 1H), 3.51-3.48 (m, 1H), 2.60 (br s, 1H), |
| | 1.95-1.92 (m, 1H), 1.60 (br d, J = 6.2 Hz, 1H), 1.26 (d, J = 6.4 Hz, 3H) |
| 113 | 1H NMR (400 MHz, DMSO-d6) δ = 8.20 (s, 1H), 7.54 (d, J = 8.6 Hz, 2H), |
| | 7.42 (s, 1H), 7.24-7.23 (m, 1H), 7.22-7.20 (m, 2H), 7.18 (s, 1H), 7.05 (s, |
| | 1H), 5.73 (d, J = 4.4 Hz, 1H), 5.52 (s, 2H), 5.28 (d, J = 5.4 Hz, 1H), 5.11 (br |
| | d, J = 4.8 Hz, 1H), 5.07 (d, J = 5.6 Hz, 1H), 4.12 (d, J = 5.6 Hz, 1H), 3.89 (br |
| | d, J = 6.2 Hz, 1H), 3.83 (br dd, J = 2.4, 11.4 Hz, 2H), 3.26-3.21 (m, 4H), |
| | 1.94-1.88 (m, 1H), 1.61 (br d, J = 12.8 Hz, 2H), 1.25 (d, J = 6.4 Hz, 3H), |
| | 1.21-1.15 (m, 2H) |
| 114 | 1H NMR (400 MHz, DMSO-d6) δ = 8.42 (d, J = 2.2 Hz, 1H), 8.23 (s, 1H), |
| | 8.06 (dd, J = 2.4, 8.5 Hz, 1H), 7.90-7.52 (t, J = 72.8 Hz, 1H), 7.40 (t, J = 5.6 |
| | Hz, 1H), 7.12 (d, J = 8.4 Hz, 1H), 5.69 (d, J = 4.4 Hz, 1H), 5.54 (s, 2H), 5.29 |
| | (d, J = 5.2 Hz, 1H), 5.14-5.10 (m, 1H), 5.08 (d, J = 5.8 Hz, 1H), 4.13 (q, J = |
| | 5.6 Hz, 1H), 3.88 (t, J = 6.0 Hz, 1H), 3.45 (br t, J = 6.4 Hz, 2H), 2.70-2.61 |
| | (m, 2H), 2.61-2.56 (m, 1H), 2.40-2.32 (m, 2H), 1.25 (d, J = 6.2 Hz, 3H) |
| 115 | 1H NMR (400 MHz, DMSO-d6) δ = 8.42 (d, J = 2.4 Hz, 1H), 8.24-8.22 (m, |
| | 1H), 8.07 (dd, J = 2.4, 8.4 Hz, 1H), 7.90-7.53 (m, 1H), 7.32 (br d, J = 2.6 |
| | Hz, 1H), 7.14-7.09 (m, 1H), 5.72 (d, J = 4.4 Hz, 1H), 5.57-5.53 (m, 2H), |
| | 5.31-5.27 (m, 1H), 5.14-5.10 (m, 1H), 5.09-5.06 (m, 1H), 4.13 (q, J = 5.6 |
| | Hz, 1H), 3.89 (quin, J = 6.2 Hz, 1H), 3.36-3.35 (m, 2H), 2.28-1.97 (m, 4H), |
| | 1.95-1.82 (m, 2H), 1.57-1.47 (m, 1H), 1.28-1.24 (m, 3H) |
| 116 | 1H NMR (400 MHz, DMSO-d6) δ = 8.42 (d, J = 1.8 Hz, 1H), 8.22 (s, 1H), |
| | 8.08-8.04 (m, 1H), 7.90-7.53 (t, J = 48.8 Hz, 1H), 7.30 (s, 1H), 7.12 (d, J = |
| | 8.6 Hz, 1H), 5.72 (d, J = 4.2 Hz, 1H), 5.54 (s, 2H), 5.30 (d, J = 5.4 Hz, 1H), |
| | 5.11 (br d, J = 5.0 Hz, 1H), 5.08 (d, J = 5.8 Hz, 1H), 4.12 (d, J = 5.6 Hz, 1H), |
| | 3.88 (t, J = 6.0 Hz, 1H), 3.24 (br t, J = 6.0 Hz, 2H), 1.99 (br d, J = 9.0 Hz, |
| | 2H), 1.79 (br d, J = 9.6 Hz, 5H), 1.25 (d, J = 6.4 Hz, 3H), 1.18 (br d, J = 12.4 |
| | Hz, 2H) |
| 117 | 1H NMR (400 MHz, DMSO-d6) δ = 8.24 (br d, J = 8.6 Hz, 2H), 8.16 (s, 1H), |
| | 7.70 (br d, J = 8.6 Hz, 2H), 7.04 (br d, J = 7.4 Hz, 1H), 5.80 (d, J = 4.4 Hz, |
| | 1H), 5.69 (s, 2H), 5.24 (d, J = 5.4 Hz, 1H), 5.08-5.02 (m, 2H), 4.11 (q, J = |
| | 5.6 Hz, 1H), 4.04 (s, 1H), 3.86 (quin, J = 5.8 Hz, 1H), 3.75-3.67 (m, 1H), |
| | 1.71 (br d, J = 5.6 Hz, 4H), 1.58 (br d, J = 13.4 Hz, 2H), 1.41-1.36 (m, 2H), |
| | 1.25 (br d, J = 6.4 Hz, 3H), 1.12 (s, 3H) |
| 118 | 1H NMR (400 MHz, DMSO-d6) δ = 8.25 (d, J = 8.6 Hz, 2H), 8.17 (s, 1H), |
| | 7.71 (br d, J = 8.6 Hz, 2H), 6.94 (br d, J = 7.6 Hz, 1H), 5.75 (br d, J = 4.0 Hz, |
| | 1H), 5.70 (s, 2H), 5.28 (d, J = 5.4 Hz, 1H), 5.11-5.06 (m, 2H), 4.31 (s, 1H), |
| | 4.15-4.10 (m, 1H), 3.88 (quin, J = 6.2 Hz, 1H), 3.83-3.76 (m, 1H), 1.86 (br |
| | s, 2H), 1.61-1.52 (m, 3H), 1.47 (br t, J = 7.6 Hz, 3H), 1.25 (br d, J = 6.4 Hz, |
| | 3H), 1.16 (s, 3H) |
| 119 | 1H NMR (400 MHz, DMSO-d6) δ = 8.19-8.17 (m, 1H), 7.87-7.84 (m, 2H), |
| | 7.64 (d, J = 8.4 Hz, 2H), 7.45 (d, J = 6.8 Hz, 1H), 5.77 (d, J = 4.4 Hz, 1H), |
| | 5.63 (s, 2H), 5.26 (d, J = 5.4 Hz, 1H), 5.12-5.07 (m, 1H), 5.05 (d, J = 5.8 |
| | Hz, 1H), 4.97 (s, 1H), 4.12 (q, J = 5.8 Hz, 1H), 3.94-3.84 (m, 2H), 2.37 (br |
| | dd, J = 6.4, 11.3 Hz, 2H), 2.12-2.05 (m, 2H), 1.28-1.24 (m, 6H) |
| 120 | 1H NMR (400 MHz, DMSO-d6) δ = 8.18 (s, 1H), 7.86 (d, J = 8.4 Hz, 2H), |
| | 7.64 (d, J = 8.4 Hz, 2H), 7.41 (d, J = 7.0 Hz, 1H), 5.74 (d, J = 4.4 Hz, 1H), |
| | 5.63 (s, 2H), 5.29 (d, J = 5.2 Hz, 1H), 5.12 (d, J = 4.8 Hz, 1H), 5.07 (d, J = |
| | 5.8 Hz, 1H), 4.84 (s, 1H), 4.47 (d, J = 7.6 Hz, 1H), 4.14 (d, J = 5.6 Hz, 1H), |
| | 3.88 (t, J = 6.2 Hz, 1H), 2.35-2.29 (m, 2H), 2.05 (br dd, J = 5.0, 6.8 Hz, 2H), |
| | 1.28 (s, 3H), 1.25 (d, J = 6.4 Hz, 3H) |
| 121 | 1H NMR (400 MHz, DMSO-d6) δ = 8.19 (s, 1H), 7.54 (d, J = 8.6 Hz, 2H), |
| | 7.42 (s, 1H), 7.23 (s, 1H), 7.19 (br d, J = 8.6 Hz, 2H), 7.05 (s, 1H), 6.98 (br |
| | d, J = 7.8 Hz, 1H), 5.79 (d, J = 4.4 Hz, 1H), 5.51 (s, 2H), 5.23 (d, J = 5.4 Hz, |
| | 1H), 5.08-5.02 (m, 2H), 4.11 (q, J = 5.8 Hz, 1H), 4.03 (s, 1H), 3.86 (quin, J = |
| | 5.8 Hz, 1H), 3.71-3.64 (m, 1H), 1.74-1.66 (m, 4H), 1.56 (br d, J = 12.6 |
| | Hz, 2H), 1.39 (br dd, J = 4.2, 12.4 Hz, 2H), 1.25 (d, J = 6.4 Hz, 3H), 1.10 (s, |
| | 3H) |
| 122 | 1H NMR (400 MHz, DMSO-d6) δ = 8.19 (s, 1H), 7.54 (br d, J = 8.6 Hz, 2H), |
| | 7.41 (s, 1H), 7.23 (s, 1H), 7.19 (br d, J = 8.4 Hz, 2H), 7.04 (s, 1H), 6.87 (br |
| | d, J = 7.4 Hz, 1H), 5.74 (d, J = 4.2 Hz, 1H), 5.52 (s, 2H), 5.27 (d, J = 5.4 Hz, |
| | 1H), 5.10-5.05 (m, 2H), 4.30 (s, 1H), 4.12 (q, J = 5.4 Hz, 1H), 3.88 (quin, J = |
| | 6.0 Hz, 1H), 3.77 (br dd, J = 2.8, 4.4 Hz, 1H), 1.84 (br s, 2H), 1.58-1.53 |
| | (m, 2H), 1.49-1.43 (m, 4H), 1.25 (br d, J = 6.4 Hz, 3H), 1.15 (s, 3H) |
| 123 | 1H NMR (400 MHz, DMSO-d6) δ = 8.20 (s, 1H), 7.54 (br d, J = 7.6 Hz, 2H), |
| | 7.42 (s, 1H), 7.23 (br s, J = 74 Hz, 1H), 7.19 (br d, J = 8.0 Hz, 2H), 5.76 (br |
| | d, J = 3.6 Hz, 1H), 5.51 (s, 2H), 5.26 (d, J = 5.2 Hz, 1H), 5.09 (br d, J = 4.8 |
| | Hz, 1H), 5.04 (d, J = 5.8 Hz, 1H), 4.96 (s, 1H), 4.12 (br d, J = 5.8 Hz, 1H), |
| | 3.92-3.84 (m, 2H), 2.35 (br dd, J = 6.4, 10.7 Hz, 2H), 2.11-2.04 (m, 2H), |
| | 1.27-1.23 (m, 6H) |
| 124 | 1H NMR (400 MHz, DMSO-d6) δ = 8.20 (s, 1H), 7.54 (br d, J = 8.4 Hz, 2H), |
| | 7.37 (br d, J = 7.0 Hz, 1H), 7.23 (t, J = 74 Hz, 1H), 7.19 (br d, J = 8.4 Hz, 2H), |
| | 5.73 (d, J = 4.2 Hz, 1H), 5.51 (s, 2H), 5.28 (d, J = 5.2 Hz, 1H), 5.12 (br d, J = |
| | 5.0 Hz, 1H), 5.06 (d, J = 5.8 Hz, 1H), 4.83 (s, 1H), 4.45 (br d, J = 7.4 Hz, |
| | 1H), 4.14 (br d, J = 5.6 Hz, 1H), 3.88 (br t, J = 6.0 Hz, 1H), 2.31 (br dd, J = |
| | 7.2, 11.9 Hz, 2H), 2.07-1.99 (m, 2H), 1.28-1.23 (m, 6H) |
| 125 | 1H NMR (400 MHz, DMSO-d6) δ = 8.17 (s, 1H), 7.77-7.74 (m, 2H), 7.68- |
| | 7.65 (m, 2H), 7.02 (d, J = 7.6 Hz, 1H), 5.80 (d, J = 4.4 Hz, 1H), 5.63 (s, |
| | 2H), 5.23 (d, J = 5.4 Hz, 1H), 5.07-5.03 (m, 2H), 4.11 (q, J = 5.6 Hz, 1H), |
| | 4.03 (s, 1H), 3.86 (quin, J = 6.2 Hz, 1H), 3.74-3.66 (m, 1H), 1.72-1.68 (m, |
| | 4H), 1.57 (br d, J = 12.6 Hz, 2H), 1.39 (br dd, J = 4.8, 12.8 Hz, 2H), 1.25 (d, |
| | J = 6.4 Hz, 3H), 1.11 (s, 3H) |
| 126 | 1H NMR (400 MHz, DMSO-d6) δ = 8.18 (s, 1H), 7.77-7.74 (m, 2H), 7.68- |
| | 7.65 (m, 2H), 6.92 (br d, J = 7.6 Hz, 1H), 5.75 (d, J = 4.4 Hz, 1H), 5.64 (s, |
| | 2H), 5.28 (d, J = 5.4 Hz, 1H), 5.08 (d, J = 5.8 Hz, 2H), 4.31 (s, 1H), 4.12 (q, |
| | J = 5.6 Hz, 1H), 3.91-3.85 (m, 1H), 3.79 (br s, 1H), 1.85 (br s, 2H), 1.59- |
| | 1.54 (m, 2H), 1.50-1.44 (m, 4H), 1.25 (d, J = 6.4 Hz, 3H), 1.16 (s, 3H) |
| 127 | 1H NMR (400 MHz, DMSO-d6) δ = 8.17 (s, 1H), 7.50-7.47 (m, 2H), 7.45- |
| | 7.42 (m, 2H), 6.97 (br d, J = 7.8 Hz, 1H), 5.78 (d, J = 4.4 Hz, 1H), 5.51 (s, |
| | 2H), 5.22 (d, J = 5.4 Hz, 1H), 5.05-5.01 (m, 2H), 4.10 (q, J = 5.6 Hz, 1H), |
| | 4.01 (s, 1H), 3.85 (t, J = 6.2 Hz, 1H), 3.71-3.65 (m, 1H), 1.70-1.66 (m, |
| | 4H), 1.56 (br d, J = 12.6 Hz, 2H), 1.38 (br dd, J = 4.8, 12.8 Hz, 2H), 1.24 (d, |
| | J = 6.4 Hz, 3H), 1.10 (s, 3H) |
| 128 | 1H NMR (400 MHz, DMSO-d6) δ = 8.19 (d, J = 1.4 Hz, 1H), 7.51-7.48 (m, |
| | 2H), 7.46-7.43 (m, 2H), 6.88 (br d, J = 7.8 Hz, 1H), 5.74 (d, J = 3.6 Hz, 1H), |
| | 5.52 (s, 2H), 5.27 (d, J = 4.4 Hz, 1H), 5.08-5.05 (m, 2H), 4.30 (s, 1H), 4.15- |
| | 4.10 (m, 1H), 3.90-3.85 (m, 1H), 3.81-3.74 (m, 1H), 1.85 (br d, J = 7.6 |
| | Hz, 2H), 1.57-1.54 (m, 2H), 1.47-1.43 (m, 4H), 1.25 (br d, J = 6.0 Hz, 3H), |
| | 1.15 (s, 3H) |
| 129 | 1H NMR (400 MHz, DMSO-d6) δ = 8.18 (s, 1H), 7.54-7.51 (m, 2H), 7.23- |
| | 7.18 (m, 2H), 6.96 (br d, J = 7.8 Hz, 1H), 5.78 (d, J = 4.4 Hz, 1H), 5.49 (s, |
| | 2H), 5.21 (d, J = 5.4 Hz, 1H), 5.05-5.01 (m, 2H), 4.12-4.08 (m, 1H), 3.85 |
| | (t, J = 6.2 Hz, 1H), 3.71-3.65 (m, 1H), 1.68 (br d, J = 7.4 Hz, 4H), 1.55 (br |
| | d, J = 12.8 Hz, 2H), 1.38-1.33 (m, 2H), 1.24 (d, J = 6.4 Hz, 3H), 1.09 (s, |
| | 3H) |
| 130 | 1H NMR (400 MHz, DMSO-d6) δ = 8.20 (s, 1H), 7.54 (dd, J = 5.8, 8.4 Hz, |
| | 2H), 7.22 (t, J = 8.8 Hz, 2H), 6.88 (br d, J = 7.8 Hz, 1H), 5.74 (d, J = 4.2 Hz, |
| | 1H), 5.51 (s, 2H), 5.28 (d, J = 5.4 Hz, 1H), 5.08 (d, J = 5.8 Hz, 2H), 4.30 (s, |
| | 1H), 4.13 (q, J = 5.6 Hz, 1H), 3.88 (t, J = 6.2 Hz, 1H), 3.77 (br s, 1H), 1.83 |
| | (br s, 2H), 1.57 (br s, 2H), 1.48-1.43 (m, 4H), 1.25 (d, J = 6.4 Hz, 3H), 1.15 |
| | (s, 3H) |
| 131 | 1H NMR (400 MHz, DMSO-d6) δ = 8.17 (s, 1H), 7.85 (d, J = 8.4 Hz, 2H), |
| | 7.64 (d, J = 8.4 Hz, 2H), 6.94-6.89 (m, 1H), 5.75 (d, J = 4.4 Hz, 1H), 5.64 |
| | (s, 2H), 5.27 (d, J = 5.4 Hz, 1H), 5.10-5.06 (m, 2H), 4.30 (s, 1H), 4.12 (q, J = |
| | 5.6 Hz, 1H), 3.91-3.86 (m, 1H), 3.83-3.74 (m, 1H), 1.89-1.83 (m, 2H), |
| | 1.59-1.54 (m, 2H), 1.52-1.45 (m, 4H), 1.25 (d, J = 6.4 Hz, 3H), 1.16 (s, |
| | 3H) |
| 132 | 1H NMR (400 MHz, DMSO-d6) δ = 8.16 (s, 1H), 7.85 (d, J = 8.2 Hz, 2H), |
| | 7.63 (d, J = 8.2 Hz, 2H), 7.03 (d, J = 7.6 Hz, 1H), 5.80 (d, J = 4.2 Hz, 1H), |
| | 5.63 (s, 2H), 5.23 (d, J = 5.4 Hz, 1H), 5.07-5.03 (m, 2H), 4.11 (q, J = 5.8 |
| | Hz, 1H), 4.04 (s, 1H), 3.86 (t, J = 6.0 Hz, 1H), 3.74-3.65 (m, 1H), 1.75- |
| | 1.68 (m, 4H), 1.60-1.54 (m, 2H), 1.42-1.34 (m, 2H), 1.25 (d, J = 6.4 Hz, |
| | 3H), 1.11 (s, 3H) |
| 133 | 1H NMR (400 MHz, DMSO-d6) δ = 8.25 (d, J = 8.8 Hz, 2H), 8.18 (s, 1H), |
| | 7.71 (d, J = 8.6 Hz, 2H), 7.46 (br d, J = 6.6 Hz, 1H), 5.79-5.76 (m, 1H), 5.69 |
| | (s, 2H), 5.26 (d, J = 5.2 Hz, 1H), 5.10 (q, J = 5.2 Hz, 1H), 5.05 (d, J = 5.8 Hz, |
| | 1H), 4.97 (s, 1H), 4.12 (q, J = 5.8 Hz, 1H), 3.95-3.85 (m, 2H), 2.37 (br dd, |
| | J = 6.8, 10.9 Hz, 2H), 2.13-2.06 (m, 2H), 1.28-1.24 (m, 6H) |
| 134 | 1H NMR (400 MHz, DMSO-d6) δ = 8.25 (d, J = 8.6 Hz, 2H), 8.18 (s, 1H), |
| | 7.71 (d, J = 8.6 Hz, 2H), 7.42 (d, J = 7.0 Hz, 1H), 5.74 (d, J = 4.4 Hz, 1H), |
| | 5.69 (s, 2H), 5.29 (d, J = 5.4 Hz, 1H), 5.12 (q, J = 5.2 Hz, 1H), 5.07 (d, J = |
| | 5.6 Hz, 1H), 4.84 (s, 1H), 4.52-4.44 (m, 1H), 4.14 (q, J = 5.8 Hz, 1H), 3.88 |
| | (quin, J = 6.0 Hz, 1H), 2.35-2.29 (m, 2H), 2.09-2.02 (m, 2H), 1.29-1.23 |
| | (m, 6H) |
| 136 | 1H NMR (400 MHz, DMSO-d6) δ = 8.16 (d, J = 2.2 Hz, 1H), 8.08 (s, 1H), |
| | 8.01 (dd, J = 2.4, 8.5 Hz, 1H), 7.42 (d, J = 8.6 Hz, 1H), 7.05 (d, J = 7.0 Hz, |
| | 1H), 5.70 (d, J = 4.2 Hz, 1H), 5.41 (br d, J = 8.6 Hz, 2H), 5.21 (d, J = 5.4 Hz, |
| | 1H), 5.08 (q, J = 5.0 Hz, 1H), 5.02 (d, J = 5.8 Hz, 1H), 4.40-4.35 (m, 2H), |
| | 4.14 (q, J = 5.8 Hz, 2H), 3.84 (t, J = 6.0 Hz, 1H), 3.41-3.35 (m, 2H), 3.18- |
| | 3.14 (m, 2H), 3.00 (br t, J = 6.0 Hz, 2H), 2.29-2.18 (m, 4H), 1.23 (d, J = 6.2 |
| | Hz, 3H) |
| 137 | 1H NMR (400 MHz, DMSO-d6) δ = 8.05 (d, J = 2.2 Hz, 1H), 8.01 (s, 1H), |
| | 7.98-7.95 (m, 1H), 7.38 (d, J = 8.4 Hz, 1H), 7.07 (br d, J = 6.2 Hz, 1H), 5.63 |
| | (d, J = 4.0 Hz, 1H), 5.27 (br s, 2H), 5.05-4.99 (m, 2H), 4.43-4.31 (m, 5H), |
| | 4.09 (br d, J = 5.8 Hz, 1H), 3.81-3.78 (m, 1H), 2.97 (br d, J = 5.2 Hz, 2H), |
| | 2.33-2.30 (m, 2H), 2.06 (br d, J = 8.4 Hz, 2H), 1.25 (s, 3H), 1.18 (d, J = 6.4 |
| | Hz, 3H) |
| 138 | 1H NMR (400 MHz, DMSO-d6) δ-8.15 (d, J = 1.8 Hz, 1H), 8.07 (s, 1H), |
| | 8.02 (dd, J = 2.4, 8.4 Hz, 1H), 7.63 (s, 1H), 7.43-7.42 (m, 1H), 7.26 (br t, J = |
| | 5.4 Hz, 1H), 5.64 (d, J = 4.4 Hz, 1H), 5.37 (br s, 2H), 5.21 (d, J = 5.4 Hz, |
| | 1H), 5.06-4.98 (m, 3H), 4.48-4.44 (m, 2H), 4.37 (br d, J = 5.6 Hz, 2H), |
| | 4.09 (d, J = 5.8 Hz, 1H), 3.82 (t, J = 6.0 Hz, 1H), 3.77 (s, 3H), 3.04 (br t, J = |
| | 5.4 Hz, 2H), 1.22 (d, J = 6.2 Hz, 3H) |
| 139 | 1H NMR (400 MHz, DMSO-d6) δ = 8.09-8.04 (m, 2H), 8.04-7.99 (m, 1H), |
| | 7.42 (d, J = 8.4 Hz, 1H), 6.62 (d, J = 6.8 Hz, 1H), 5.73-5.68 (m, 1H), 5.43- |
| | 5.30 (m, 2H), 5.21 (d, J = 5.3 Hz, 1H), 5.05-4.99 (m, 2H), 4.45-4.36 (m, |
| | 2H), 4.33-4.30 (m, 1H), 4.14-4.06 (m, 1H), 3.88-3.81 (m, 1H), 3.80-3.72 |
| | (m, 1H), 3.03-2.97 (m, 2H), 1.97-1.89 (m, 2H), 1.63-1.58 (m, 2H), 1.57- |
| | 1.45 (m, 4H), 1.25-1.22 (m, 3H), 1.18 (s, 3H) |
| 140 | 1H NMR (400 MHz, DMSO-d6) δ = 8.06 (s, 2H), 8.01 (dd, J = 2.2, 8.3 Hz, |
| | 1H), 7.42 (d, J = 8.6 Hz, 1H), 6.68 (d, J = 7.2 Hz, 1H), 5.69 (d, J = 4.2 Hz, |
| | 1H), 5.45-5.31 (m, 2H), 5.19 (d, J = 5.4 Hz, 1H), 5.04-4.98 (m, 2H), 4.59 |
| | (d, J = 4.4 Hz, 1H), 4.44-4.34 (m, 2H), 4.11 (q, J = 5.8 Hz, 1H), 3.83 (quin, |
| | J = 6.2 Hz, 1H), 3.72-3.63 (m, 1H), 3.42 (br dd, J = 4.6, 9.6 Hz, 1H), 3.00 |
| | (br t, J = 5.4 Hz, 2H), 2.07-1.99 (m, 2H), 1.90 (br d, J = 9.6 Hz, 2H), 1.41- |
| | 1.29 (m, 4H), 1.23 (d, J = 6.4 Hz, 3H) |
| 141 | 1H NMR (400 MHz, DMSO-d6) δ = 8.09 (s, 1H), 8.06 (s, 1H), 8.01 (br d, J = |
| | 8.6 Hz, 1H), 7.43 (d, J = 8.2 Hz, 1H), 7.15 (br d, J = 5.4 Hz, 1H), 5.70 (d, |
| | J = 4.0 Hz, 1H), 5.41-5.34 (m, 2H), 5.19 (d, J = 4.8 Hz, 1H), 5.08-5.03 (m, |
| | 1H), 5.00-4.95 (m, 2H), 4.44-4.37 (m, 2H), 4.11 (q, J = 6.0 Hz, 1H), 3.88- |
| | 3.80 (m, 2H), 3.01-2.99 (m, 2H), 2.40 (br d, J = 5.6 Hz, 2H), 2.12-2.07 |
| | (m, 2H), 1.33 (s, 3H), 1.23 (d, J = 6.2 Hz, 3H) |
| 142 | 1H NMR (400 MHz, DMSO-d6) δ = 9.43-9.40 (m, 1H), 8.21-8.18 (m, 1H), |
| | 8.16-8.12 (m, 1H), 8.06-8.01 (m, 1H), 8.00-7.96 (m, 1H), 7.63-7.58 (m, |
| | 1H), 7.49-7.43 (m, 1H), 5.85-5.81 (m, 1H), 5.48-5.39 (m, 2H), 5.34-5.31 |
| | (m, 1H), 5.24-5.20 (m, 1H), 5.07-5.03 (m, 1H), 4.48-4.41 (m, 2H), 4.21- |
| | 4.16 (m, 1H), 3.91-3.87 (m, 4H), 3.09-3.04 (m, 2H), 1.26-1.24 (m, 3H) |
| 143 | 1H NMR (400 MHz, DMSO-d6) δ = 8.16 (d, J = 2.0 Hz, 1H), 8.08 (s, 1H), |
| | 8.01 (dd, J = 2.2, 8.4 Hz, 1H), 7.42 (d, J = 8.4 Hz, 1H), 7.10 (s, 1H), 5.65 (d, |
| | J = 4.4 Hz, 1H), 5.34 (s, 2H), 5.25 (br d, J = 4.2 Hz, 1H), 5.08 (br d, J = 3.4 |
| | Hz, 1H), 5.05-4.99 (m, 1H), 4.46-4.38 (m, 2H), 4.12 (br d, J = 5.2 Hz, 1H), |
| | 3.85 (t, J = 6.4 Hz, 1H), 3.54 (br s, 2H), 3.23-3.19 (m, 2H), 3.04-3.00 (m, |
| | 2H), 2.99 (s, 3H), 2.12-2.06 (m, 2H), 1.24 (d, J = 6.4 Hz, 3H) |
| 144 | 1H NMR (400 MHz, DMSO-d6) δ = 8.09-8.03 (m, 2H), 8.00 (dd, J = 2.4, |
| | 8.4 Hz, 1H), 7.41 (d, J = 8.5 Hz, 1H), 6.70 (d, J = 7.1 Hz, 1H), 5.73 (d, J = |
| | 4.1 Hz, 1H), 5.39 (br d, J = 12.6 Hz, 2H), 5.17 (d, J = 5.4 Hz, 1H), 5.03-4.97 |
| | (m, 2H), 4.38 (br d, J = 6.5 Hz, 2H), 4.12-4.05 (m, 2H), 3.83 (t, J = 6.1 Hz, |
| | 1H), 3.70-3.62 (m, 1H), 2.99 (br t, J = 5.6 Hz, 2H), 1.84-1.71 (m, 2H), 1.63 |
| | (br d, J = 12.9 Hz, 2H), 1.46 (br dd, J = 3.9, 13.1 Hz, 4H), 1.23 (d, J = 6.3 |
| | Hz, 3H), 1.16 (s, 3H) |
| 145 | 1H NMR (400 MHz, DMSO-d6) δ = 8.10 (d, J = 1.8 Hz, 1H), 8.07 (s, 1H), |
| | 8.02 (dd, J = 2.4, 8.4 Hz, 1H), 7.44 (d, J = 8.4 Hz, 1H), 6.69 (s, 2H), 5.67 (d, |
| | J = 4.2 Hz, 1H), 5.29 (s, 2H), 5.22 (d, J = 5.4 Hz, 1H), 5.01-4.96 (m, 2H), |
| | 4.42 (br t, J = 5.6 Hz, 2H), 4.12 (q, J = 6.0 Hz, 1H), 3.83 (quin, J = 6.2 Hz, |
| | 1H), 3.00 (br t, J = 5.8 Hz, 2H), 1.25 (d, J = 6.4 Hz, 3H) |
| 146 | 1H NMR (400 MHz, DMSO-d6) δ = 8.10 (d, J = 2.0 Hz, 1H), 8.07 (s, 1H), |
| | 8.01 (dd, J = 2.2, 8.4 Hz, 1H), 7.42 (d, J = 8.4 Hz, 1H), 6.93-6.90 (m, 1H), |
| | 5.65 (d, J = 4.4 Hz, 1H), 5.34 (s, 2H), 5.23-5.22 (m, 1H), 5.04-5.00 (m, |
| | 2H), 4.42-4.40 (m, 2H), 4.11-4.09 (m, 1H), 3.85-3.82 (m, 1H), 3.45-3.41 |
| | (m, 4H), 3.27 (s, 3H), 3.02-2.99 (m, 2H), 1.91-1.84 (m, 2H), 1.24 (d, J = |
| | 6.4 Hz, 3H) |
| 147 | 1H NMR (400 MHz, CDCl₃) δ = 8.70 (s, 1H), 8.26-8.11 (m, 3H), 7.66 (br |
| | d, J = 7.8 Hz, 2H), 5.92 (br d, J = 4.6 Hz, 1H), 5.33 (br s, 1H), 4.82-4.66 (m, |
| | 2H), 4.61 (br s, 1H), 4.43 (br s, 1H), 4.21 (br s, 1H), 2.95 (br s, 1H), 1.40 (br |
| | d, J = 6.2 Hz, 3H) |
| 148 | 1H NMR (400 MHz, DMSO-d6) δ = 8.07 (s, 2H), 8.01 (dd, J = 2.0, 8.5 Hz, |
| | 1H), 7.42 (d, J = 8.4 Hz, 1H), 6.86 (d, J = 7.2 Hz, 1H), 5.71 (d, J = 4.0 Hz, |
| | 1H), 5.44-5.32 (m, 2H), 5.21 (d, J = 5.2 Hz, 1H), 5.08-5.04 (m, 1H), 5.01 |
| | (d, J = 5.8 Hz, 1H), 4.44-4.35 (m, 2H), 4.13 (q, J = 5.8 Hz, 1H), 3.93 (br d, |
| | J = 10.8 Hz, 3H), 3.84 (t, J = 5.8 Hz, 1H), 3.47 (br t, J = 11.2 Hz, 2H), 3.00 |
| | (br t, J = 5.6 Hz, 2H), 2.01-1.94 (m, 2H), 1.59 (dt, J = 4.4, 12.0 Hz, 2H), |
| | 1.23 (d, J = 6.4 Hz, 3H) |
| 149 | 1H NMR (400 MHz, DMSO-d6) δ = 8.10 (d, J = 2.0 Hz, 1H), 8.06 (s, 1H), |
| | 8.00 (dd, J = 2.2, 8.4 Hz, 1H), 7.42 (d, J = 8.4 Hz, 1H), 6.72-6.70 (m, 1H), |
| | 5.70 (d, J = 4.4 Hz, 1H), 5.34-5.20 (m, 2H), 5.20 (d, J = 5.2 Hz, 1H), 5.05- |
| | 4.99 (m, 2H), 4.43 (br s, 2H), 4.42-4.07 (m, 2H), 3.86-3.82 (m, 1H), 3.02- |
| | 2.99 (m, 2H), 1.26-1.22 (m, 9H) |
| 150 | 1H NMR (400 MHz, DMSO-d6) δ = 8.11 (br s, 1H), 8.06 (s, 1H), 8.03-7.99 |
| | (m, 1H), 7.43 (br d, J = 8.4 Hz, 1H), 6.96-6.88 (m, 1H), 5.65 (d, J = 4.2 Hz, |
| | 1H), 5.38-5.31 (m, 2H), 5.09-5.03 (m, 1H), 4.46-4.40 (m, 2H), 4.15-4.07 |
| | (m, 1H), 3.88-3.81 (m, 1H), 3.43-3.38 (m, 4H), 3.01 (br t, J = 5.8 Hz, 2H), |
| | 1.25-1.20 (m, 6H) |
| 153 | 1H NMR (400 MHz, DMSO-d6) δ = 8.05 (s, 1H), 7.70 (s, 1H), 7.62-7.58 |
| | (m, 1H), 7.34 (d, J = 7.8 Hz, 1H), 6.75 (d, J = 6.4 Hz, 1H), 5.72 (d, J = 4.2 |
| | Hz, 1H), 5.24 (br s, 2H), 5.18 (d, J = 5.4 Hz, 1H), 5.02 (br d, J = 4.6 Hz, 1H), |
| | 4.99 (d, J = 5.8 Hz, 1H), 4.45-4.39 (m, 2H), 4.22-4.15 (m, 1H), 4.12 (q, J = |
| | 5.8 Hz, 1H), 3.87-3.81 (m, 1H), 2.96 (br t, J = 5.4 Hz, 2H), 2.02 (br dd, J = |
| | 4.8, 6.8 Hz, 2H), 1.74-1.69 (m, 2H), 1.59 (br d, J = 2.2 Hz, 4H), 1.24 (d, |
| | J = 6.4 Hz, 3H) |
| 154 | 1H NMR (400 MHz, DMSO-d6) δ = 8.05 (s, 1H), 7.71 (s, 1H), 7.60 (br d, J = |
| | 7.8 Hz, 1H), 7.34 (br d, J = 7.8 Hz, 1H), 6.69 (br d, J = 7.0 Hz, 1H), 5.70 |
| | (d, J = 4.0 Hz, 1H), 5.23 (br s, 2H), 5.20 (d, J = 5.4 Hz, 1H), 5.03 (br d, J = |
| | 4.6 Hz, 1H), 5.00 (br d, J = 6.0 Hz, 1H), 4.41 (br s, 2H), 4.14-4.06 (m, 2H), |
| | 3.84 (br t, J = 6.2 Hz, 1H), 2.96 (br t, J = 5.2 Hz, 2H), 1.26 (br s, 3H), 1.25- |
| | 1.23 (m, 6H). |
| 155 | 1H NMR (400 MHz, DMSO-d6) δ = 8.06 (s, 1H), 7.72 (s, 1H), 7.60 (d, J = |
| | 7.6 Hz, 1H), 7.35 (d, J = 8.0 Hz, 1H), 6.85 (t, J = 5.2 Hz, 1H), 5.67 (d, J = 4.2 |
| | Hz, 1H), 5.30-5.18 (m, 3H), 5.05-4.97 (m, 2H), 4.57 (t, J = 5.2 Hz, 1H), |
| | 4.40 (br t, J = 5.4 Hz, 2H), 4.09 (q, J = 5.8 Hz, 1H), 3.83 (quin, J = 6.2 Hz, |
| | 1H), 3.53 (q, J = 6.0 Hz, 2H), 3.43 (q, J = 6.4 Hz, 2H), 2.96 (br t, J = 5.8 Hz, |
| | 2H), 1.79 (quin, J = 6.4 Hz, 2H), 1.25 (d, J = 6.4 Hz, 3H). |
| 160 | 1H NMR (400 MHz, DMSO-d6) δ = 8.04 (s, 1H), 7.68 (s, 1H), 7.61-7.55 |
| | (m, 1H), 7.32 (d, J = 8.0 Hz, 1H), 6.88 (t, J = 5.4 Hz, 1H), 5.63 (d, J = 4.4 |
| | Hz, 1H), 5.24 (br s, 2H), 5.20 (d, J = 5.4 Hz, 1H), 5.04-4.98 (m, 2H), 4.38 |
| | (br s, 2H), 4.08 (q, J = 5.8 Hz, 1H), 3.84-3.78 (m, 1H), 3.41 (q, J = 5.8 Hz, |
| | 4H), 3.25 (s, 3H), 2.94 (br t, J = 5.8 Hz, 2H), 1.85 (quin, J = 6.6 Hz, 2H), 1.22 |
| | (d, J = 6.4 Hz, 3H) |
| 164 | 1H NMR (400 MHz, DMSO-d6) δ = 8.07 (s, 1H), 7.72 (s, 1H), 7.60 (dd, J = |
| | 1.4, 7.8 Hz, 1H), 7.35 (d, J = 8.0 Hz, 1H), 7.19 (d, J = 2.4 Hz, 1H), 5.61 (d, J = |
| | 4.0 Hz, 1H), 5.29 (br d, J = 2.2 Hz, 2H), 5.18 (d, J = 5.0 Hz, 1H), 5.07- |
| | 5.01 (m, 1H), 4.98 (d, J = 5.8 Hz, 1H), 4.48-4.39 (m, 2H), 4.16-4.08 (m, |
| | 1H), 3.82 (quin, J = 6.2 Hz, 1H), 2.97 (br t, J = 5.8 Hz, 2H), 2.82 (dq, J = 3.6, |
| | 6.8 Hz, 1H), 1.22 (d, J = 6.4 Hz, 3H), 0.77-0.70 (m, 2H), 0.58-0.52 (m, |
| | 2H) |
| 167 | 1H NMR (400 MHz, DMSO-d6) δ = 8.07 (s, 1H), 7.73 (s, 1H), 7.60 (d, J = |
| | 7.8 Hz, 1H), 7.35 (d, J = 7.8 Hz, 1H), 6.76 (t, J = 5.4 Hz, 1H), 5.70 (d, J = 4.4 |
| | Hz, 1H), 5.27-5.21 (m, 3H), 5.03 (d, J = 5.9 Hz, 1H), 4.97 (q, J = 5.4 Hz, |
| | 1H), 4.80 (t, J = 5.4 Hz, 1H), 4.44-4.37 (m, 2H), 4.08 (q, J = 5.8 Hz, 1H), |
| | 3.89-3.81 (m, 1H), 3.68-3.61 (m, 2H), 3.44 (q, J = 5.8 Hz, 2H), 2.96 (br t, |
| | J = 5.8 Hz, 2H), 1.25 (d, J = 6.4 Hz, 3H) |
| 170 | 1H NMR (400 MHz, DMSO-d6) δ = 8.05 (s, 1H), 7.18-7.11 (m, 4H), 6.79 |
| | (d, J = 7.0 Hz, 1H), 5.71 (d, J = 4.2 Hz, 1H), 5.26-5.19 (m, 3H), 5.08-5.03 |
| | (m, 1H), 5.01 (d, J = 5.8 Hz, 1H), 4.35 (br s, 2H), 4.14 (q, J = 5.8 Hz, 1H), |
| | 3.94-3.90 (m, 2H), 3.85 (t, J = 6.2 Hz, 1H), 3.45 (dt, J = 1.8, 11.6 Hz, 2H), |
| | 3.13-3.08 (m, 1H), 2.87 (br t, J = 5.8 Hz, 2H), 2.02-1.93 (m, 2H), 1.65- |
| | 1.54 (m, 2H), 1.24 (d, J = 6.4 Hz, 3H) |
| 171 | 1H NMR (400 MHz, DMSO-d6) δ = 8.04 (s, 1H), 7.17-7.12 (m, 4H), 6.62 |
| | (d, J = 7.0 Hz, 1H), 5.69 (d, J = 4.0 Hz, 1H), 5.26 (br d, J = 5.0 Hz, 2H), 5.19 |
| | (d, J = 5.4 Hz, 1H), 5.04-4.98 (m, 2H), 4.57 (d, J = 4.4 Hz, 1H), 4.34 (br s, |
| | 2H), 4.12 (q, J = 6.0 Hz, 1H), 3.83 (quin, J = 6.2 Hz, 1H), 3.71-3.63 (m, |
| | 1H), 3.42 (br dd, J = 3.8, 4.8 Hz, 1H), 2.88 (br t, J = 5.8 Hz, 2H), 2.07-2.00 |
| | (m, 2H), 1.89 (br d, J = 9.6 Hz, 2H), 1.40-1.28 (m, 4H), 1.23 (d, J = 6.4 Hz, |
| | 3H) |
| 173 | 1H NMR (400 MHz, DMSO-d6) δ = 8.60 (s, 1H), 8.57 (s, 1H), 7.58 (d, J = |
| | 8.5 Hz, 2H), 7.43-7.05 (m, 3H), 5.94 (d, J = 5.0 Hz, 1H), 5.67-5.60 (m, |
| | 2H), 5.53-5.42 (m, 1H), 5.30-5.15 (m, 1H), 4.69 (br t, J = 4.5 Hz, 1H), 4.04- |
| | 3.96 (m, 2H), 1.32 (d, J = 6.0 Hz, 3H) |
| 174 | 1H NMR (400 MHz, DMSO-d6) δ = 8.54 (s, 1H), 8.47 (s, 1H), 7.78 (dd, J = |
| | 6.8, 7.9 Hz, 1H), 7.60 (d, J = 10.3 Hz, 1H), 7.54 (d, J = 8.1 Hz, 1H), 6.04 (d, |
| | J = 4.5 Hz, 1H), 5.78 (s, 2H), 4.79 (t, J = 4.7 Hz, 1H), 4.17-4.11 (m, 2H), |
| | 1.43 (d, J = 6.2 Hz, 3H) |
| 176 | 1H NMR (400 MHz, DMSO-d6) δ = 8.79 (s, 1H), 8.67 (s, 1H), 7.54-7.50 |
| | (m, 2H), 7.38-7.00 (m, 3H), 5.94 (d, J = 4.8 Hz, 1H), 5.49 (d, J = 5.6 Hz, |
| | 1H), 5.21 (d, J = 5.2 Hz, 1H), 4.72-4.66 (m, 3H), 4.03-3.98 (m, 2H), 1.32 |
| | (d, J = 6.0 Hz, 3H) |
| 178 | 1H NMR (400 MHz, DMSO-d6) δ = 8.78 (s, 1H), 8.68 (s, 1H), 7.80-7.75 |
| | (m, 2H), 7.70-7.64 (m, 2H), 5.94 (d, J = 4.9 Hz, 1H), 5.51 (br d, J = 5.0 Hz, |
| | 1H), 5.23 (br d, J = 2.9 Hz, 1H), 4.75 (s, 2H), 4.71-4.66 (m, 1H), 4.04-3.97 |
| | (m, 2H), 1.32 (d, J = 6.0 Hz, 3H) |
| 179 | 1H NMR (400 MHz, DMSO-d6) δ = 8.56 (d, J = 9.4 Hz, 2H), 8.18 (d, J = 8.7 |
| | Hz, 2H), 7.64 (d, J = 8.7 Hz, 2H), 5.93 (d, J = 5.0 Hz, 1H), 5.50 (d, J = 5.7 |
| | Hz, 1H), 5.23 (d, J = 4.6 Hz, 1H), 4.84 (t, J = 6.7 Hz, 2H), 4.67 (q, J = 4.9 |
| | Hz, 1H), 4.02-3.96 (m, 2H), 3.31 (br s, 2H), 1.31 (d, J = 6.1 Hz, 3H) |
| 180 | 1H NMR (400 MHz, DMSO-d6) δ = 8.71-8.55 (m, 2H), 7.75-7.61 (m, 2H), |
| | 7.50-7.38 (m, 2H), 6.03-5.91 (m, 1H), 5.69 (br d, J = 4.2 Hz, 2H), 5.57- |
| | 5.41 (m, 1H), 5.32-5.13 (m, 1H), 4.71 (br d, J = 4.2 Hz, 1H), 4.02 (br d, J = |
| | 4.2 Hz, 2H), 1.34 (br d, J = 5.0 Hz, 3H) |
| 181 | 1H NMR (400 MHz, DMSO-d6) δ = 8.06 (s, 1H), 7.25-7.21 (m, 1H), 7.20- |
| | 7.11 (m, 3H), 6.98 (d, J = 6.8 Hz, 1H), 5.70 (d, J = 4.0 Hz, 1H), 5.27 (br s, |
| | 2H), 5.21 (d, J = 5.4 Hz, 1H), 5.12-5.05 (m, 1H), 5.01 (d, J = 5.8 Hz, 1H), |
| | 4.34 (br t, J = 5.6 Hz, 2H), 4.20-4.05 (m, 2H), 3.84 (quin, J = 6.2 Hz, 1H), |
| | 3.37 (br s, 2H), 3.22-3.13 (m, 2H), 2.88 (br t, J = 5.8 Hz, 2H), 2.31 (br dd, |
| | J = 3.0, 14.4 Hz, 2H), 2.25-2.10 (m, 2H), 1.24 (d, J = 6.4 Hz, 3H) |
| 182 | 1H NMR (400 MHz, DMSO-d6) δ = 8.61 (s, 1H), 8.57 (s, 1H), 7.62 (q, J = |
| | 8.3 Hz, 4H), 7.19-6.89 (m, 1H), 5.95 (d, J = 4.9 Hz, 1H), 5.71 (s, 2H), 5.54- |
| | 5.15 (m, 2H), 4.69 (t, J = 4.7 Hz, 1H), 4.03-3.98 (m, 2H), 1.32 (d, J = 6.0 |
| | Hz, 3H) |
| 184 | 1H NMR (400 MHz, DMSO-d6) δ = 8.79 (s, 1H), 8.66 (s, 1H), 7.49 (d, J = |
| | 8.0 Hz, 2H), 7.28 (d, J = 8.2 Hz, 2H), 5.94 (d, J = 4.8 Hz, 1H), 5.50 (d, J = |
| | 5.6 Hz, 1H), 5.22 (d, J = 5.0 Hz, 1H), 4.72-4.68 (m, 1H), 4.66 (s, 2H), 4.03- |
| | 3.98 (m, 4H), 1.32 (br d, J = 6.0 Hz, 3H) |
| 185 | 1H NMR (400 MHz, DMSO-d6) δ = 8.57 (br s, 1H), 8.38 (s, 1H), 8.25-8.13 |
| | (m, 3H), 7.58 (d, J = 8.6 Hz, 2H), 5.86 (d, J = 5.0 Hz, 1H), 5.52-5.38 (m, |
| | 1H), 5.19 (br s, 1H), 4.82 (br s, 2H), 4.68 (br s, 1H), 4.02-3.93 (m, 2H), 1.30 |
| | (d, J = 6.0 Hz, 3H) |
| 186 | 1H NMR (400 MHz, DMSO-d6) δ = 8.80 (s, 1H), 8.68 (s, 1H), 7.93 (d, J = |
| | 8.2 Hz, 2H), 7.65 (d, J = 8.2 Hz, 2H), 5.94 (d, J = 4.8 Hz, 1H), 5.51 (d, J = |
| | 5.5 Hz, 1H), 5.23 (br d, J = 5.0 Hz, 1H), 4.75 (s, 2H), 4.70 (br d, J = 5.0 Hz, |
| | 1H), 4.01 (br d, J = 4.0 Hz, 2H), 2.65 (s, 3H), 1.32 (br d, J = 6.1 Hz, 3H) |
| 187 | 1H NMR (400 MHz, DMSO-d6) δ = 8.59 (d, J = 12.8 Hz, 2H), 7.59-7.44 |
| | (m, 4H), 5.94 (d, J = 5.0 Hz, 1H), 5.64 (s, 2H), 5.53-5.14 (m, 2H), 4.69 (t, J = |
| | 4.8 Hz, 1H), 4.04-3.91 (m, 2H), 1.32 (d, J = 6.0 Hz, 3H) |
| 193 | 1H NMR (400 MHz, DMSO-d6) δ = 8.63 (s, 1H), 8.57 (s, 1H), 8.02 (d, J = |
| | 8.4 Hz, 1H), 7.61 (s, 1H), 7.55 (d, J = 8.4 Hz, 1H), 5.95 (d, J = 5.0 Hz, 1H), |
| | 5.72 (s, 2H), 5.53 (br d, J = 4.0 Hz, 1H), 5.25 (br s, 1H), 4.70 (br d, J = 3.6 |
| | Hz, 1H), 4.04-3.97 (m, 2H), 2.53 (s, 3H), 1.32 (d, J = 6.2 Hz, 3H) |
| 195 | 1H NMR (400 MHz, DMSO-d6) δ = 8.05-8.02 (m, 1H), 7.17-7.14 (m, 2H), |
| | 7.14-7.12 (m, 2H), 6.54-6.50 (m, 1H), 5.78-5.76 (m, 1H), 5.27-5.23 (m, |
| | 2H), 5.19-5.17 (m, 1H), 5.02-4.99 (m, 1H), 4.99-4.95 (m, 1H), 4.41-4.39 |
| | (m, 1H), 4.37-4.33 (m, 2H), 4.11-4.07 (m, 1H), 3.86-3.82 (m, 1H), 3.79- |
| | 3.73 (m, 2H), 2.89-2.85 (m, 2H), 1.84-1.77 (m, 2H), 1.74-1.65 (m, 4H), |
| | 1.58-1.52 (m, 2H), 1.26-1.24 (m, 3H) |
| 196 | 1H NMR (400 MHz, DMSO-d6) δ = 8.44 (s, 1H), 8.33 (s, 1H), 8.10 (d, J = |
| | 2.0 Hz, 1H), 8.06 (dd, J = 2.4, 8.4 Hz, 1H), 7.58 (d, J = 8.4 Hz, 1H), 5.90 (d, |
| | J = 4.8 Hz, 1H), 5.65-4.97 (m, 4H), 4.70-4.40 (m, 3H), 3.97 (q, J = 4.2 Hz, |
| | 2H), 3.09 (br t, J = 5.6 Hz, 2H), 1.30 (d, J = 6.0 Hz, 3H) |
| 198 | 1H NMR (400 MHz, DMSO-d6) δ = 8.06 (s, 1H), 7.72 (s, 1H), 7.60 (d, J = |
| | 7.6 Hz, 1H), 7.34 (d, J = 7.8 Hz, 1H), 6.91 (t, J = 5.2 Hz, 1H), 5.65 (d, J = 4.1 |
| | Hz, 1H), 5.26-5.20 (m, 3H), 5.05 (q, J = 5.2 Hz, 1H), 5.00 (d, J = 5.8 Hz, |
| | 1H), 4.41 (br t, J = 5.4 Hz, 2H), 4.12 (q, J = 5.8 Hz, 1H), 3.87-3.80 (m, 1H), |
| | 3.44-3.35 (m, 2H), 2.96 (br t, J = 5.6 Hz, 2H), 1.25-1.21 (m, 6H) |
| 200 | 1H NMR (400 MHz, DMSO-d6) δ = 8.62 (s, 1H), 8.58 (s, 1H), 7.51 (dd, J = |
| | 6.9, 8.6 Hz, 2H), 5.95 (d, J = 5.0 Hz, 1H), 5.61 (s, 2H), 5.48 (br d, J = 5.4 Hz, |
| | 1H), 5.21 (br d, J = 4.6 Hz, 1H), 4.69 (br d, J = 4.6 Hz, 1H), 4.04-3.97 (m, |
| | 2H), 1.32 (d, J = 6.1 Hz, 3H) |
| 201 | 1H NMR (400 MHz, DMSO-d6) δ = 8.60 (d, J = 11.7 Hz, 2H), 7.66-7.58 |
| | (m, 1H), 7.48 (td, J = 8.4, 10.7 Hz, 1H), 7.40 (br d, J = 3.5 Hz, 1H), 5.94 (d, |
| | J = 5.0 Hz, 1H), 5.61 (s, 2H), 5.53 (br s, 1H), 5.30-5.21 (m, 1H), 4.69 (br s, |
| | 1H), 4.03-3.97 (m, 2H), 1.31 (br d, J = 6.2 Hz, 3H) |
| 202 | 1H NMR (400 MHz, DMSO-d6) δ = 8.59 (d, J = 7.6 Hz, 2H), 7.58 (dd, J = |
| | 5.6, 8.4 Hz, 2H), 7.24 (t, J = 8.8 Hz, 2H), 5.94 (d, J = 5.0 Hz, 1H), 5.62 (s, |
| | 2H), 5.49 (d, J = 5.6 Hz, 1H), 5.22 (br d, J = 4.8 Hz, 1H), 4.69 (q, J = 4.8 Hz, |
| | 1H), 4.05-3.95 (m, 2H), 1.31 (d, J = 6.0 Hz, 3H) |
| 204 | 1H NMR (400 MHz, DMSO-d6) δ = 8.62 (s, 1H), 8.57 (s, 1H), 7.81-7.75 |
| | (m, 2H), 7.74-7.69 (m, 2H), 5.95 (d, J = 4.9 Hz, 1H), 5.75 (s, 2H), 5.49 (d, |
| | J = 5.7 Hz, 1H), 5.21 (d, J = 5.1 Hz, 1H), 4.70 (q, J = 5.0 Hz, 1H), 4.05-3.96 |
| | (m, 2H), 1.32 (br d, J = 6.0 Hz, 3H) |
| 205 | 1H NMR (400 MHz, DMSO-d6) δ = 8.83 (s, 1H), 8.64 (s, 1H), 8.59 (s, 1H), |
| | 8.38-8.32 (m, 2H), 5.95 (d, J = 5.0 Hz, 1H), 5.85 (s, 2H), 5.49 (d, J = 5.6 |
| | Hz, 1H), 5.22 (d, J = 4.8 Hz, 1H), 4.73-4.65 (m, 1H), 4.00 (br d, J = 4.4 Hz, |
| | 2H), 1.32 (d, J = 6.4 Hz, 3H) |
| 206 | 1H NMR (400 MHz, DMSO-d6) δ = 8.58 (s, 1H), 8.57 (s, 1H), 7.38 (d, J = |
| | 8.1 Hz, 2H), 7.10 (d, J = 8.1 Hz, 2H), 5.93 (d, J = 5.0 Hz, 1H), 5.57 (s, 2H), |
| | 5.49 (d, J = 5.6 Hz, 1H), 5.24-5.19 (m, 1H), 4.68 (q, J = 5.0 Hz, 1H), 4.03- |
| | 3.96 (m, 2H), 1.96-1.87 (m, 1H), 1.31 (br d, J = 6.1 Hz, 3H), 0.98-0.91 (m, |
| | 2H), 0.69-0.63 (m, 2H) |
| 207 | 1H NMR (400 MHz, DMSO-d6) δ = 8.79 (s, 1H), 8.66 (s, 1H), 7.43 (d, J = |
| | 8.7 Hz, 2H), 6.99 (d, J = 8.7 Hz, 2H), 5.94 (d, J = 4.9 Hz, 1H), 5.49 (d, J = |
| | 5.6 Hz, 1H), 5.21 (d, J = 5.0 Hz, 1H), 4.77-4.66 (m, 3H), 4.63 (s, 2H), 4.04- |
| | 3.97 (m, 2H), 1.32 (d, J = 6.0 Hz, 3H) |
| 211 | 1H NMR (400 MHz, DMSO-d6) δ = 8.58 (d, J = 6.2 Hz, 2H), 7.40 (d, J = 7.8 |
| | Hz, 2H), 7.21 (d, J = 7.8 Hz, 2H), 5.94 (d, J = 4.8 Hz, 1H), 5.59 (s, 2H), 5.48 |
| | (d, J = 5.8 Hz, 1H), 5.20 (d, J = 5.0 Hz, 1H), 4.68 (q, J = 5.0 Hz, 1H), 4.00 |
| | (br d, J = 3.8 Hz, 2H), 2.31 (s, 3H), 1.31 (br d, J = 6.0 Hz, 3H) |
| 212 | 1H NMR (400 MHz, DMSO-d6) δ = 8.04 (s, 1H), 7.18-7.12 (m, 4H), 6.64 |
| | (br d, J = 7.0 Hz, 1H), 5.70 (d, J = 4.0 Hz, 1H), 5.25-5.18 (m, 3H), 5.05- |
| | 4.98 (m, 2H), 4.38 (br s, 2H), 4.17-4.10 (m, 1H), 4.10-4.00 (m, 1H), 3.88- |
| | 3.80 (m, 1H), 2.88 (br t, J = 5.2 Hz, 2H), 1.27-1.23 (m, 9H) |
| 213 | 1H NMR (400 MHz, DMSO-d6) δ = 8.37 (s, 1H), 8.27 (s, 1H), 8.14 (d, J = |
| | 8.8 Hz, 2H), 7.57 (d, J = 8.8 Hz, 2H), 5.89 (d, J = 4.8 Hz, 1H), 5.58 (br d, J = |
| | 9.4 Hz, 1H), 5.47 (br d, J = 5.4 Hz, 1H), 5.20 (br s, 1H), 4.63 (q, J = 4.8 |
| | Hz, 1H), 4.01-3.93 (m, 2H), 3.30-3.07 (m, 4H), 1.94 (br d, J = 11.4 Hz, |
| | 2H), 1.68 (dq, J = 3.4, 12.6 Hz, 2H), 1.30 (d, J = 6.0 Hz, 3H) |
| 217 | 1H NMR (400 MHz, DMSO-d6) δ = 8.63 (s, 1H), 8.58 (s, 1H), 7.62 (s, 1H), |
| | 7.59 (s, 2H), 5.95 (d, J = 4.9 Hz, 1H), 5.64 (s, 2H), 5.62-5.07 (m, 2H), 4.69 |
| | (t, J = 4.6 Hz, 1H), 4.00 (br d, J = 4.5 Hz, 2H), 1.32 (d, J = 6.0 Hz, 3H) |
| 224 | 1H NMR (400 MHz, DMSO-d6) δ = 8.80 (s, 1H), 8.67 (s, 1H), 7.46 (d, J = |
| | 7.0 Hz, 2H), 7.34-7.29 (m, 2H), 7.27-7.22 (m, 1H), 5.94 (d, J = 4.8 Hz, |
| | 1H), 5.54 (br d, J = 4.8 Hz, 1H), 5.25 (br s, 1H), 4.72-4.66 (m, 3H), 4.03- |
| | 3.98 (m, 2H), 1.32 (d, J = 6.2 Hz, 3H) |
| 226 | 1H NMR (400 MHz, DMSO-d6) δ = 8.62 (s, 1H), 8.58 (s, 1H), 7.80 (d, J = |
| | 2.0 Hz, 1H), 7.68 (d, J = 8.3 Hz, 1H), 7.51 (dd, J = 2.0, 8.3 Hz, 1H), 5.95 (d, |
| | J = 4.9 Hz, 1H), 5.64 (s, 2H), 5.53 (br d, J = 4.9 Hz, 1H), 5.25 (br s, 1H), 4.69 |
| | (br d, J = 4.1 Hz, 1H), 4.03-3.98 (m, 2H), 1.32 (d, J = 6.3 Hz, 3H) |
| 230 | 1H NMR (400 MHz, DMSO-d6) δ = 8.59 (d, J = 3.2 Hz, 2H), 7.82-7.79 (m, |
| | 2H), 7.32-7.29 (m, 1H), 5.94 (d, J = 5.0 Hz, 1H), 5.62 (s, 2H), 5.53-5.15 |
| | (m, 2H), 4.68 (t, J = 4.6 Hz, 1H), 4.02-3.98 (m, 2H), 3.90 (s, 3H), 1.31 (d, J = |
| | 6.0 Hz, 3H) |
| 231 | 1H NMR (400 MHz, DMSO-d6) δ = 8.57 (s, 2H), 7.46 (br d, J = 8.6 Hz, 2H), |
| | 6.96 (br d, J = 8.8 Hz, 2H), 5.93 (d, J = 4.9 Hz, 1H), 5.56 (s, 2H), 5.47 (d, J = |
| | 5.8 Hz, 1H), 5.25-5.15 (m, 1H), 4.68 (br d, J = 5.4 Hz, 1H), 4.05-3.94 |
| | (m, 2H), 3.76 (s, 3H), 1.36-1.26 (m, 3H) |
| 234 | 1H NMR (400 MHz, DMSO-d6) δ = 8.60 (d, J = 14.4 Hz, 2H), 7.86 (s, 1H), |
| | 7.62 (s, 2H), 5.95 (d, J = 5.0 Hz, 1H), 5.67 (s, 2H), 5.50 (br s, 1H), 5.23 (br |
| | s, 1H), 4.69 (br s, 1H), 4.03-3.97 (m, 2H), 1.32 (d, J = 6.3 Hz, 3H) |
| 235 | 1H NMR (400 MHz, DMSO-d6) δ = 8.55 (s, 1H), 8.41 (s, 1H), 7.50 (d, J = |
| | 8.2 Hz, 1H), 7.26 (s, 1H), 7.20 (dd, J = 1.9, 8.2 Hz, 1H), 6.03 (d, J = 4.5 Hz, |
| | 1H), 5.67 (s, 2H), 4.77 (t, J = 4.7 Hz, 1H), 4.17-4.10 (m, 2H), 2.43 (s, 3H), |
| | 1.45-1.41 (m, 3H) |
| 236 | 1H NMR (400 MHz, DMSO-d6) δ = 8.57 (s, 2H), 7.47 (d, J = 8.6 Hz, 2H), |
| | 7.00 (d, J = 8.6 Hz, 2H), 5.94 (d, J = 5.0 Hz, 1H), 5.57 (s, 2H), 5.52-5.15 |
| | (m, 2H), 4.82-4.78 (m, 1H), 4.70-4.66 (m, 2H), 4.30-4.26 (m, 1H), 4.23- |
| | 4.18 (m, 1H), 4.04-3.95 (m, 2H), 1.31 (d, J = 6.0 Hz, 3H) |
| 238 | 1H NMR (400 MHz, DMSO-d6) δ = 8.60 (d, J = 6.1 Hz, 2H), 7.53-7.45 (m, |
| | 1H), 7.44-7.35 (m, 1H), 5.94 (d, J = 4.9 Hz, 1H), 5.69 (s, 2H), 5.48 (d, J = |
| | 5.6 Hz, 1H), 5.21 (d, J = 5.1 Hz, 1H), 4.68 (q, J = 5.1 Hz, 1H), 4.04-3.95 |
| | (m, 2H), 1.31 (br d, J = 6.0 Hz, 3H) |
| 241 | 1H NMR (400 MHz, DMSO-d6) δ = 8.55 (d, J = 11.4 Hz, 2H), 7.47 (d, J = |
| | 8.6 Hz, 2H), 7.30 (br d, J = 8.0 Hz, 2H), 5.93 (d, J = 4.9 Hz, 1H), 5.47 (d, J = |
| | 5.8 Hz, 1H), 5.20 (d, J = 5.0 Hz, 1H), 4.78 (t, J = 6.8 Hz, 2H), 4.68 (q, J = |
| | 4.9 Hz, 1H), 4.04-3.95 (m, 2H), 3.18 (t, J = 6.7 Hz, 2H), 1.31 (d, J = 6.0 Hz, |
| | 3H) |
| 242 | 1H NMR (400 MHz, DMSO-d6) δ = 8.83 (s, 1H), 8.67 (s, 1H), 7.90 (br d, J = |
| | 7.7 Hz, 1H), 7.70 (br d, J = 7.8 Hz, 1H), 7.68-7.57 (m, 2H), 7.55-7.49 |
| | (m, 1H), 7.48-7.43 (m, 1H), 5.95 (br d, J = 2.2 Hz, 1H), 5.50 (br s, 1H), 5.21 |
| | (br s, 1H), 5.08 (s, 2H), 4.70 (br s, 1H), 4.01 (br s, 2H), 1.32 (br d, J = 4.0 |
| | Hz, 3H) |
| 245 | 1H NMR (400 MHz, DMSO-d6) δ = 8.62 (s, 1H), 8.56 (s, 1H), 7.98 (d, J = |
| | 8.2 Hz, 2H), 7.63 (d, J = 8.2 Hz, 2H), 5.95 (d, J = 5.0 Hz, 1H), 5.74 (s, 2H), |
| | 5.50 (br s, 1H), 5.22 (br s, 1H), 4.69 (br d, J = 4.6 Hz, 1H), 4.03-3.97 (m, |
| | 2H), 2.58 (s, 3H), 1.32 (d, J = 6.2 Hz, 3H) |
| 246 | 1H NMR (400 MHz, DMSO-d6) δ = 8.70-8.53 (m, 2H), 8.53-8.51 (m, 1H), |
| | 7.49-7.32 (m, 3H), 7.32-7.03 (m, 2H), 5.95 (br d, J = 4.8 Hz, 1H), 5.66 (s, |
| | 2H), 5.47 (br d, J = 5.6 Hz, 1H), 5.20 (br d, J = 4.2 Hz, 1H), 4.69 (br d, J = |
| | 5.0 Hz, 1H), 4.00 (br s, 2H), 1.32 (br d, J = 5.6 Hz, 3H) |
| 249 | 1H NMR (400 MHz, DMSO-d6) δ = 8.56 (s, 1H), 8.54 (s, 1H), 7.78 (d, J = |
| | 7.8 Hz, 2H), 7.56 (d, J = 7.8 Hz, 2H), 5.93 (d, J = 4.9 Hz, 1H), 5.48 (br d, J = |
| | 5.6 Hz, 1H), 5.21 (br d, J = 4.6 Hz, 1H), 4.81 (t, J = 6.5 Hz, 2H), 4.67 (br |
| | d, J = 4.6 Hz, 1H), 3.99 (br d, J = 4.2 Hz, 2H), 3.25 (br t, J = 6.5 Hz, 2H), |
| | 1.31 (br d, J = 5.6 Hz, 3H) |
| 250 | 1H NMR (400 MHz, DMSO-d6) δ = 8.62 (s, 1H), 8.58 (s, 1H), 8.04 (d, J = |
| | 1.0 Hz, 1H), 7.86-7.82 (m, 1H), 7.80-7.76 (m, 1H), 5.95 (d, J = 5.0 Hz, |
| | 1H), 5.71 (s, 2H), 5.49 (d, J = 5.7 Hz, 1H), 5.22 (d, J = 5.1 Hz, 1H), 4.69 (q, |
| | J = 5.0 Hz, 1H), 4.05-3.96 (m, 2H), 1.32 (d, J = 6.0 Hz, 3H) |
| 251 | 1H NMR (400 MHz, DMSO-d6) δ = 8.99 (d, J = 2.1 Hz, 1H), 8.89 (d, J = 1.8 |
| | Hz, 1H), 8.80 (s, 1H), 8.68 (s, 1H), 8.41 (t, J = 2.0 Hz, 1H), 5.94 (d, J = 4.8 |
| | Hz, 1H), 5.62-5.45 (m, 1H), 5.35-5.15 (m, 1H), 4.72-4.67 (m, 3H), 4.00 |
| | (br d, J = 5.0 Hz, 2H), 1.32 (br d, J = 6.1 Hz, 3H) |
| 252 | 1H NMR (400 MHz, DMSO-d6) δ = 8.33 (s, 1H), 8.25 (br s, 1H), 8.15 (d, J = |
| | 8.7 Hz, 2H), 7.94 (br s, 1H), 7.54 (br d, J = 8.2 Hz, 2H), 5.84 (d, J = 4.8 |
| | Hz, 1H), 5.45 (br d, J = 4.2 Hz, 1H), 5.18 (br s, 1H), 4.66 (br s, 1H), 4.02- |
| | 3.92 (m, 2H), 3.77 (br s, 2H), 3.08 (t, J = 7.1 Hz, 2H), 1.30 (d, J = 6.1 Hz, |
| | 3H) |
| 255 | 1H NMR (400 MHz, DMSO-d6) δ = 9.68 (s, 1H), 8.53 (br s, 1H), 8.38 (s, |
| | 1H), 8.22 (s, 1H), 7.98 (d, J = 8.4 Hz, 2H), 7.52 (d, J = 8.0 Hz, 2H), 5.86 (d, |
| | J = 4.9 Hz, 1H), 5.45 (d, J = 5.6 Hz, 1H), 5.17 (br d, J = 4.8 Hz, 1H), 4.85- |
| | 4.63 (m, 3H), 4.03-3.91 (m, 2H), 1.30 (d, J = 6.0 Hz, 3H) |
| 257 | 1H NMR (400 MHz, DMSO-d6) δ = 8.96 (d, J = 1.6 Hz, 1H), 8.75 (s, 1H), |
| | 8.71-8.67 (m, 1H), 8.24 (dd, J = 2.2, 8.2 Hz, 1H), 7.75 (d, J = 8.2 Hz, 1H), |
| | 5.94 (d, J = 4.9 Hz, 1H), 5.49 (d, J = 5.6 Hz, 1H), 5.20 (d, J = 5.1 Hz, 1H), |
| | 4.86 (s, 2H), 4.69 (q, J = 5.0 Hz, 1H), 4.03-3.98 (m, 2H), 1.32 (d, J = 6.1 |
| | Hz, 3H) |
| 260 | 1H NMR (400 MHz, DMSO-d6) δ = 8.86-8.36 (m, 2H), 7.32 (br d, J = 3.6 |
| | Hz, 2H), 6.04-5.87 (m, 1H), 5.76-5.59 (m, 2H), 5.57-5.43 (m, 1H), 5.33- |
| | 5.10 (m, 1H), 4.81-4.59 (m, 1H), 4.00 (br s, 2H), 1.45-1.22 (m, 3H) |
| 264 | 1H NMR (400 MHz, DMSO-d6) δ = 8.21 (s, 1H), 7.49-7.46 (m, 2H), 7.40- |
| | 7.36 (m, 2H), 7.14 (br d, J = 4.8 Hz, 1H), 5.65 (d, J = 4.4 Hz, 1H), 5.52 (s, |
| | 2H), 5.29 (d, J = 5.3 Hz, 1H), 5.10 (d, J = 4.9 Hz, 1H), 5.05 (d, J = 5.8 Hz, |
| | 1H), 4.11 (d, J = 5.6 Hz, 1H), 3.87 (t, J = 6.1 Hz, 1H), 2.87 (d, J = 4.4 Hz, |
| | 3H), 1.26 (d, J = 6.4 Hz, 3H) |
| 269 | 1H NMR (400 MHz, MeOD-d4) δ = 8.57 (s, 1H), 8.41 (s, 1H), 7.58-7.52 |
| | (m, 2H), 6.03 (d, J = 4.5 Hz, 1H), 5.79 (s, 2H), 4.77 (t, J = 4.6 Hz, 1H), 4.17- |
| | 4.10 (m, 2H), 1.42 (d, J = 6.1 Hz, 3H) |
| 270 | 1H NMR (400 MHz, DMSO-d6) δ = 8.90 (s, 1H), 8.80 (s, 1H), 8.69 (s, 1H), |
| | 8.17 (br d, J = 8.1 Hz, 1H), 7.84 (d, J = 8.4 Hz, 1H), 5.94 (d, J = 4.9 Hz, 1H), |
| | 5.50 (br d, J = 5.3 Hz, 1H), 5.22 (br d, J = 4.6 Hz, 1H), 4.77 (s, 2H), 4.70- |
| | 4.67 (m, 1H), 4.00 (br d, J = 4.0 Hz, 2H), 1.31 (br d, J = 6.0 Hz, 3H) |
| 272 | 1H NMR (400 MHz, DMSO-d6) δ = 8.79 (s, 1H), 8.66 (s, 1H), 7.18 (d, J = |
| | 7.0 Hz, 2H), 5.94 (d, J = 4.9 Hz, 1H), 5.50 (br d, J = 5.5 Hz, 1H), 5.22 (br d, |
| | J = 4.8 Hz, 1H), 4.69 (br d, J = 5.0 Hz, 1H), 4.57 (s, 2H), 4.06-3.96 (m, 2H), |
| | 2.17 (d, J = 1.8 Hz, 6H), 1.32 (d, J = 6.1 Hz, 3H) |
| 273 | 1H NMR (400 MHz, DMSO-d6) δ = 8.79 (s, 1H), 8.67 (s, 1H), 7.97-7.94 |
| | (m, 1H), 7.72-7.67 (m, 2H), 7.54-7.48 (m, 1H), 7.35 (s, 2H), 5.94 (d, J = |
| | 4.9 Hz, 1H), 5.50 (d, J = 5.6 Hz, 1H), 5.22 (d, J = 5.0 Hz, 1H), 4.75 (s, 2H), |
| | 4.69 (br d, J = 5.0 Hz, 1H), 4.05-3.97 (m, 2H), 1.32 (d, J = 6.2 Hz, 3H) |
| 274 | 1H NMR (400 MHz, DMSO-d6) δ = 8.60 (d, J = 10.4 Hz, 2H), 7.90 (s, 1H), |
| | 7.83 (br d, J = 7.8 Hz, 1H), 7.76-7.71 (m, 1H), 7.69-7.63 (m, 1H), 5.95 (d, |
| | J = 4.9 Hz, 1H), 5.73 (s, 2H), 5.48 (d, J = 5.6 Hz, 1H), 5.20 (d, J = 4.9 Hz, |
| | 1H), 4.69 (br d, J = 5.0 Hz, 1H), 4.04-3.97 (m, 2H), 1.32 (d, J = 6.0 Hz, 3H) |
| 275 | 1H NMR (400 MHz, DMSO-d6) δ = 8.57 (s, 1H), 8.54 (s, 1H), 8.25 (t, J = |
| | 1.8 Hz, 1H), 8.10 (dd, J = 2.2, 8.2 Hz, 1H), 7.83 (d, J = 7.8 Hz, 1H), 7.64- |
| | 7.59 (m, 1H), 5.93 (d, J = 4.8 Hz, 1H), 5.49 (d, J = 5.6 Hz, 1H), 5.22 (d, J = |
| | 5.0 Hz, 1H), 4.84 (t, J = 6.6 Hz, 2H), 4.67 (d, J = 5.2 Hz, 1H), 4.04-3.96 (m, |
| | 2H), 3.30 (br s, 2H), 1.31 (d, J = 6.0 Hz, 3H) |
| 277 | 1H NMR (400 MHz, DMSO-d6) δ = 8.54 (br s, 1H), 8.38 (s, 1H), 8.21 (s, |
| | 1H), 7.85 (d, J = 8.3 Hz, 2H), 7.58 (d, J = 8.2 Hz, 2H), 5.86 (d, J = 4.9 Hz, |
| | 1H), 5.44 (br d, J = 5.1 Hz, 1H), 5.17 (br d, J = 3.2 Hz, 1H), 4.79 (br s, 2H), |
| | 4.68 (br s, 1H), 4.02-3.93 (m, 2H), 3.16 (s, 3H), 1.30 (d, J = 6.1 Hz, 3H) |
| 281 | 1H NMR (400 MHz, DMSO-d6) δ = 9.36 (d, J = 2.6 Hz, 1H), 8.66 (s, 1H), |
| | 8.62 (dd, J = 2.6, 8.6 Hz, 1H), 8.52 (s, 1H), 7.74 (d, J = 8.7 Hz, 1H), 5.96 (d, |
| | J = 5.0 Hz, 1H), 5.87 (s, 2H), 5.56 (br s, 1H), 5.27 (br s, 1H), 4.74-4.68 (m, |
| | 1H), 4.03-3.98 (m, 2H), 1.32 (d, J = 6.2 Hz, 3H) |
| 282 | 1H NMR (400 MHz, DMSO-d6) δ = 8.53 (br s, 1H), 8.39 (s, 1H), 8.22 (s, |
| | 1H), 7.79 (d, J = 8.2 Hz, 1H), 7.66 (s, 1H), 7.48 (br d, J = 7.8 Hz, 1H), 5.86 |
| | (d, J = 4.8 Hz, 1H), 5.42 (br s, 1H), 5.16 (br s, 1H), 4.89-4.60 (m, 3H), 4.04- |
| | 3.91 (m, 2H), 1.30 (d, J = 6.2 Hz, 3H) |
| 289 | 1H NMR (400 MHz, DMSO-d6) δ = 8.81 (s, 1H), 8.68 (s, 1H), 6.02 (s, 1H), |
| | 5.95 (d, J = 4.9 Hz, 1H), 5.57-5.39 (m, 1H), 5.22 (br s, 1H), 4.72-4.66 (m, |
| | 3H), 4.04-3.97 (m, 2H), 3.77 (s, 3H), 2.04 (s, 3H), 1.32 (d, J = 6.2 Hz, 3H) |
| 291 | 1H NMR (400 MHz, DMSO-d6) δ = 8.61 (s, 1H), 8.58 (s, 1H), 8.46 (s, 1H), |
| | 7.77 (d, J = 7.9 Hz, 2H), 7.72 (s, 1H), 7.62 (d, J = 8.2 Hz, 2H), 5.95 (d, J = |
| | 4.9 Hz, 1H), 5.68 (s, 2H), 5.48 (br d, J = 2.2 Hz, 1H), 5.21 (br s, 1H), 4.69 |
| | (br s, 1H), 4.00 (br d, J = 4.4 Hz, 2H), 1.32 (br d, J = 5.7 Hz, 3H) |
| 293 | 1H NMR (400 MHz, DMSO-d6) δ = 8.55 (d, J = 11.6 Hz, 2H), 7.37 (s, 4H), |
| | 5.93 (d, J = 5.0 Hz, 1H), 5.48 (br s, 1H), 5.21 (br s, 1H), 4.80-4.72 (m, 2H), |
| | 4.68 (br s, 1H), 3.99 (br d, J = 4.4 Hz, 2H), 3.14 (t, J = 6.7 Hz, 2H), 1.31 (d, |
| | J = 6.1 Hz, 3H) |
| 297 | 1H NMR (400 MHz, DMSO-d6) δ = 8.80 (s, 1H), 8.69 (s, 1H), 7.75-7.66 |
| | (m, 4H), 7.41 (br d, J = 5.2 Hz, 1H), 5.95 (d, J = 4.8 Hz, 1H), 5.50 (br d, J = |
| | 5.4 Hz, 1H), 5.22 (br d, J = 4.0 Hz, 1H), 4.76 (s, 2H), 4.70 (br d, J = 4.4 Hz, |
| | 1H), 4.05-3.97 (m, 2H), 2.39 (d, J = 4.4 Hz, 3H), 1.33 (d, J = 6.0 Hz, 3H) |
| 299 | 1H NMR (400 MHz, MeOD-d4) δ = 9.23 (s, 1H), 9.12 (s, 1H), 8.90 (d, J = |
| | 9.4 Hz, 2H), 7.90 (d, J = 9.4 Hz, 2H), 6.71 (d, J = 4.8 Hz, 1H), 6.28 (br d, J = |
| | 5.0 Hz, 1H), 6.01 (br s, 1H), 5.48-5.41 (m, 1H), 5.18 (br dd, J = 3.2, 6.6 |
| | Hz, 2H), 4.82-4.75 (m, 2H), 4.49-4.45 (m, 4H), 2.12 (d, J = 6.0 Hz, 3H) |
| 300 | 1H NMR (400 MHz, DMSO-d6) δ = 9.07 (d, J = 1.8 Hz, 1H), 8.80 (s, 1H), |
| | 8.69 (s, 1H), 7.67 (d, J = 1.8 Hz, 1H), 5.95 (d, J = 4.8 Hz, 1H), 5.51 (d, J = |
| | 5.4 Hz, 1H), 5.23 (d, J = 5.0 Hz, 1H), 4.83 (s, 2H), 4.72-4.68 (m, 1H), 4.02 |
| | (br d, J = 3.8 Hz, 2H), 1.33 (br d, J = 6.2 Hz, 3H) |
| 306 | 1H NMR (400 MHz, DMSO-d6) δ = 8.80 (s, 1H), 8.67 (s, 1H), 7.26-7.20 |
| | (m, 1H), 7.15 (s, 1H), 7.05 (d, J = 7.5 Hz, 1H), 6.93 (dd, J = 2.1, 8.1 Hz, 1H), |
| | 5.94 (d, J = 4.9 Hz, 1H), 5.50 (d, J = 5.6 Hz, 1H), 5.22 (d, J = 4.8 Hz, 1H), |
| | 4.69 (q, J = 5.0 Hz, 1H), 4.64 (s, 2H), 4.04-3.98 (m, 2H), 3.78 (tt, J = 3.0, |
| | 5.9 Hz, 1H), 1.32 (d, J = 5.9 Hz, 3H), 0.74 (q, J = 6.0 Hz, 2H), 0.63-0.58 |
| | (m, 2H) |
| 307 | 1H NMR (400 MHz, MeOD-d4) δ = 8.56 (s, 1H), 8.47 (s, 1H), 7.91 (d, J = |
| | 8.1 Hz, 1H), 7.79 (s, 1H), 7.66 (d, J = 8.0 Hz, 1H), 6.05 (d, J = 4.4 Hz, 1H), |
| | 5.84 (s, 2H), 4.79 (t, J = 4.6 Hz, 1H), 4.18-4.11 (m, 2H), 1.43 (d, J = 6.1 Hz, |
| | 3H) |
| 308 | 1H NMR (400 MHz, DMSO-d6) δ = 9.28 (s, 1H), 8.82 (s, 1H), 8.68 (s, 1H), |
| | 8.47 (d, J = 5.7 Hz, 1H), 8.21 (s, 1H), 7.96-7.92 (m, 1H), 7.91-7.86 (m, |
| | 1H), 7.80 (d, J = 5.5 Hz, 1H), 5.94 (d, J = 4.8 Hz, 1H), 5.53-5.47 (m, 1H), |
| | 5.22 (br s, 1H), 4.89 (s, 2H), 4.72-4.67 (m, 1H), 4.00 (br d, J = 4.3 Hz, 2H), |
| | 1.32 (br d, J = 5.9 Hz, 3H) |
| 310 | 1H NMR (400 MHz, DMSO-d6) δ = 8.74 (d, J = 5.0 Hz, 2H), 7.84 (dd, J = |
| | 1.7, 7.7 Hz, 1H), 7.69-7.63 (m, 1H), 7.59 (dt, J = 1.7, 7.7 Hz, 1H), 7.54- |
| | 7.48 (m, 1H), 5.95 (d, J = 4.8 Hz, 1H), 5.56 (br d, J = 1.2 Hz, 1H), 5.26 (br s, |
| | 1H), 5.08 (s, 2H), 4.69 (br d, J = 2.8 Hz, 1H), 4.05-3.97 (m, 2H), 1.32 (d, J = |
| | 6.1 Hz, 3H) |
| 311 | 1H NMR (400 MHz, DMSO-d6) δ = 9.20 (s, 2H), 8.80 (s, 1H), 8.69 (s, 1H), |
| | 5.93 (d, J = 4.9 Hz, 1H), 5.50 (br d, J = 5.4 Hz, 1H), 5.22 (br d, J = 4.0 Hz, |
| | 1H), 4.75 (s, 2H), 4.68 (br d, J = 4.3 Hz, 1H), 4.04-3.96 (m, 2H), 1.31 (d, J = |
| | 6.1 Hz, 3H) |
| 313 | 1H NMR (400 MHz, MeOD-d4) δ = 8.53 (s, 1H), 8.40 (s, 1H), 7.19 (d, J = |
| | 7.5 Hz, 1H), 6.99 (d, J = 7.6 Hz, 1H), 6.91 (s, 1H), 6.03 (d, J = 4.4 Hz, 1H), |
| | 5.60 (s, 2H), 4.77 (s, 1H), 4.53 (t, J = 8.7 Hz, 2H), 4.15-4.10 (m, 2H), 3.20- |
| | 3.16 (m, 2H), 1.43 (d, J = 6.1 Hz, 3H) |
| 314 | 1H NMR (400 MHz, DMSO-d6) δ = 9.31 (s, 1H), 8.61-8.60 (m, 1H), 8.60 |
| | (d, J = 8.8 Hz, 1H), 8.24 (s, 1H), 7.90 (d, J = 8.5 Hz, 2H), 7.70 (d, J = 8.5 Hz, |
| | 2H), 5.95 (d, J = 5.0 Hz, 1H), 5.70 (s, 2H), 5.48 (d, J = 5.6 Hz, 1H), 5.21 (d, |
| | J = 5.0 Hz, 1H), 4.69 (q, J = 4.9 Hz, 1H), 4.03-3.97 (m, 2H), 1.32 (d, J = 6.0 |
| | Hz, 3H) |
| 315 | 1H NMR (400 MHz, DMSO-d6) δ = 8.32 (br s, 1H), 8.26 (s, 1H), 8.22 (d, J = |
| | 8.8 Hz, 2H), 7.66 (d, J = 8.8 Hz, 2H), 5.88 (d, J = 4.8 Hz, 1H), 5.44 (d, J = |
| | 5.8 Hz, 1H), 5.19-5.15 (m, 1H), 4.66-4.61 (m, 1H), 4.13-3.94 (m, 4H), |
| | 3.72 (br d, J = 4.4 Hz, 2H), 3.30 (br s, 1H), 2.52 (br s, 1H), 2.27-2.05 (m, |
| | 1H), 1.30 (d, J = 6.0 Hz, 3H) |
| 316 | 1H NMR (400 MHz, DMSO-d6) δ = 8.61 (s, 1H), 8.59 (s, 1H), 8.27 (s, 1H), |
| | 7.76 (s, 1H), 7.71-7.68 (m, 2H), 7.67-7.63 (m, 2H), 7.11 (s, 1H), 5.95 (d, |
| | J = 4.8 Hz, 1H), 5.68 (s, 2H), 5.48 (d, J = 5.6 Hz, 1H), 5.21 (br d, J = 4.3 Hz, |
| | 1H), 4.69 (br d, J = 4.6 Hz, 1H), 4.00 (br d, J = 3.5 Hz, 2H), 1.32 (br d, J = |
| | 5.4 Hz, 3H) |
| 320 | 1H NMR (400 MHz, DMSO-d6) δ = 8.36-8.26 (m, 2H), 8.20 (s, 1H), 7.10 |
| | (s, 1H), 7.03 (s, 2H), 5.84 (d, J = 4.9 Hz, 1H), 5.42 (d, J = 5.8 Hz, 1H), 5.15 |
| | (d, J = 5.3 Hz, 1H), 4.73-4.50 (m, 3H), 4.03-3.90 (m, 2H), 2.16 (d, J = 2.6 |
| | Hz, 6H), 1.30 (d, J = 6.1 Hz, 3H) |
| 321 | 1H NMR (400 MHz, DMSO-d6) δ = 9.14 (d, J = 1.8 Hz, 1H), 8.60 (d, J = 6.5 |
| | Hz, 2H), 7.85 (d, J = 1.7 Hz, 1H), 5.95 (d, J = 5.0 Hz, 1H), 5.74 (s, 2H), 5.48 |
| | (d, J = 5.7 Hz, 1H), 5.21 (d, J = 5.0 Hz, 1H), 4.69 (q, J = 5.1 Hz, 1H), 4.04- |
| | 3.97 (m, 2H), 1.32 (d, J = 6.0 Hz, 3H) |
| 322 | 1H NMR (400 MHz, DMSO-d6) δ = 8.34 (s, 2H), 8.26-8.17 (m, 1H), 7.18 |
| | (s, 1H), 7.14-7.07 (m, 2H), 5.84 (d, J = 5.0 Hz, 1H), 5.42 (d, J = 5.6 Hz, |
| | 1H), 5.15 (br d, J = 5.4 Hz, 1H), 4.75-4.63 (m, 3H), 3.97 (br t, J = 6.0 Hz, |
| | 2H), 2.81-2.77 (m, 4H), 2.00-1.94 (m, 2H), 1.30 (br d, J = 6.0 Hz, 3H) |
| 323 | 1H NMR (400 MHz, DMSO-d6) δ = 8.62 (s, 1H), 8.59 (s, 1H), 7.85-7.72 |
| | (m, 3H), 7.69-7.60 (m, 1H), 5.95 (d, J = 5.0 Hz, 1H), 5.80 (s, 2H), 5.59- |
| | 5.22 (m, 2H), 4.72-4.67 (m, 1H), 4.06-3.97 (m, 2H), 1.32 (d, J = 6.1 Hz, |
| | 3H) |
| 324 | 1H NMR (400 MHz, MeOD-d4) δ = 8.23 (s, 1H), 8.15 (d, J = 4.6 Hz, 2H), |
| | 8.12 (s, 1H), 7.58 (br d, J = 8.3 Hz, 2H), 5.96 (d, J = 4.5 Hz, 1H), 4.65-4.62 |
| | (m, 1H), 4.46-4.25 (m, 2H), 4.15-4.10 (m, 1H), 4.08-4.04 (m, 1H), 3.40 |
| | (br d, J = 2.3 Hz, 3H), 3.16 (br t, J = 7.5 Hz, 2H), 1.42 (d, J = 6.4 Hz, 3H) |
| 325 | 1H NMR (400 MHz, DMSO-d6) δ = 8.59 (s, 1H), 8.57 (s, 1H), 7.43 (d, J = |
| | 7.1 Hz, 1H), 7.37 (br t, J = 7.8 Hz, 1H), 7.08 (d, J = 8.3 Hz, 1H), 6.97 (t, J = |
| | 7.5 Hz, 1H), 5.94 (d, J = 4.9 Hz, 1H), 5.61 (s, 2H), 5.48 (d, J = 5.6 Hz, 1H), |
| | 5.21 (d, J = 4.9 Hz, 1H), 4.69 (q, J = 4.9 Hz, 1H), 4.00 (br d, J = 3.8 Hz, 2H), |
| | 3.82 (s, 3H), 1.32 (br d, J = 6.0 Hz, 3H) |
| 327 | 1H NMR (400 MHz, DMSO-d6) δ = 8.34 (s, 1H), 8.23 (s, 2H), 7.32 (d, J = |
| | 8.4 Hz, 2H), 7.21 (d, J = 8.2 Hz, 2H), 5.85 (d, J = 4.8 Hz, 1H), 5.42 (br s, |
| | 1H), 5.16 (br s, 1H), 4.65 (br s, 1H), 4.02-3.91 (m, 2H), 2.10 (br s, 1H), 1.48 |
| | (br s, 1H), 1.38-1.07 (m, 5H) |
| 328 | 1H NMR (400 MHz, DMSO-d6) δ = 8.60 (d, J = 4.5 Hz, 2H), 8.51 (d, J = 1.9 |
| | Hz, 1H), 7.88 (d, J = 8.4 Hz, 2H), 7.75 (s, 1H), 7.64 (d, J = 8.4 Hz, 2H), 6.55 |
| | (d, J = 1.5 Hz, 1H), 5.95 (d, J = 4.9 Hz, 1H), 5.67 (s, 2H), 5.48 (br s, 1H), |
| | 5.20 (br s, 1H), 4.69 (br s, 1H), 4.00 (br d, J = 4.5 Hz, 2H), 1.32 (br d, J = 5.9 |
| | Hz, 3H) |
| 331 | 1H NMR (400 MHz, DMSO-d6) δ = 8.31 (s, 1H), 8.28 (br s, 1H), 7.70 (br s, |
| | 1H), 7.10-7.05 (m, 1H), 7.03-6.99 (m, 2H), 5.85 (d, J = 4.8 Hz, 1H), 5.42 |
| | (br d, J = 5.6 Hz, 1H), 5.14 (br d, J = 4.4 Hz, 1H), 4.92-4.58 (m, 3H), 4.02- |
| | 3.94 (m, 2H), 2.36 (s, 6H), 1.30 (d, J = 6.0 Hz, 3H) |
| 332 | 1H NMR (400 MHz, DMSO-d6) δ = 8.60 (d, J = 10.4 Hz, 2H), 8.09 (s, 1H), |
| | 7.86 (s, 2H), 7.76-7.65 (m, 1H), 5.95 (d, J = 4.8 Hz, 1H), 5.76 (s, 2H), 5.47 |
| | (d, J = 5.6 Hz, 1H), 5.20 (d, J = 4.8 Hz, 1H), 4.78-4.62 (m, 1H), 4.00 (br d, |
| | J = 4.0 Hz, 2H), 3.24 (s, 3H), 1.32 (d, J = 6.0 Hz, 3H) |
| 333 | 1H NMR (400 MHz, DMSO-d6) δ = 8.60 (s, 2H), 8.05 (s, 1H), 7.83-7.76 |
| | (m, 2H), 7.29 (d, J = 8.4 Hz, 1H), 5.95 (d, J = 5.0 Hz, 1H), 5.78 (s, 2H), 5.49 |
| | (br d, J = 5.6 Hz, 1H), 5.22 (br d, J = 4.8 Hz, 1H), 4.69 (br d, J = 4.8 Hz, 1H), |
| | 4.05 (s, 3H), 4.03-3.98 (m, 2H), 1.32 (d, J = 6.0 Hz, 3H) |
| 334 | 1H NMR (400 MHz, DMSO-d6) δ = 8.48 (br s, 1H), 8.36 (s, 1H), 8.21 (s, |
| | 1H), 7.74 (d, J = 8.2 Hz, 2H), 7.49 (d, J = 8.2 Hz, 2H), 7.25 (s, 2H), 5.85 (d, |
| | J = 4.8 Hz, 1H), 5.43 (br d, J = 5.6 Hz, 1H), 5.16 (br d, J = 4.6 Hz, 1H), 4.83- |
| | 4.63 (m, 3H), 4.01-3.95 (m, 2H), 1.30 (br d, J = 5.8 Hz, 3H) |
| 337 | 1H NMR (400 MHz, DMSO-d6) δ = 8.75 (s, 1H), 8.68 (s, 1H), 8.46 (d, J = |
| | 2.4 Hz, 1H), 7.97 (dt, J = 2.4, 9.4 Hz, 1H), 5.94 (d, J = 4.8 Hz, 1H), 5.49 (d, |
| | J = 5.6 Hz, 1H), 5.21 (d, J = 5.2 Hz, 1H), 4.86 (s, 2H), 4.69 (q, J = 4.8 Hz, |
| | 1H), 4.05-3.96 (m, 2H), 1.32 (d, J = 6.0 Hz, 3H) |
| 338 | 1H NMR (400 MHz, DMSO-d6) δ = 8.59 (d, J = 1.6 Hz, 2H), 7.83 (s, 1H), |
| | 7.68 (d, J = 8.0 Hz, 1H), 7.50 (dd, J = 1.2, 8.1 Hz, 1H), 5.94 (d, J = 4.9 Hz, |
| | 1H), 5.74 (s, 2H), 5.47 (d, J = 5.5 Hz, 1H), 5.19 (br d, J = 4.8 Hz, 1H), 4.69 |
| | (q, J = 4.9 Hz, 1H), 4.02-3.98 (m, 2H), 2.61 (s, 3H), 1.32 (d, J = 6.1 Hz, 3H) |
| 340 | 1H NMR (400 MHz, DMSO-d6) δ = 8.58 (d, J = 18.1 Hz, 2H), 6.92 (s, 2H), |
| | 5.93 (d, J = 4.9 Hz, 1H), 5.65-5.57 (m, 2H), 5.47 (br d, J = 5.6 Hz, 1H), 5.19 |
| | (br d, J = 4.6 Hz, 1H), 4.68 (br d, J = 4.6 Hz, 1H), 4.00 (br d, J = 3.8 Hz, 2H), |
| | 2.35 (s, 6H), 2.24 (s, 3H), 1.31 (br d, J = 6.0 Hz, 3H) |
| 342 | 1H NMR (400 MHz, DMSO-d6) δ = 8.80 (s, 1H), 8.69 (s, 1H), 8.32 (d, J = |
| | 8.6 Hz, 1H), 7.97 (dd, J = 8.3, 12.6 Hz, 2H), 7.76 (t, J = 7.6 Hz, 1H), 7.68 (d, |
| | J = 8.6 Hz, 1H), 7.61-7.56 (m, 1H), 5.95 (d, J = 4.9 Hz, 1H), 5.51 (br d, J = |
| | 5.3 Hz, 1H), 5.23 (br d, J = 3.5 Hz, 1H), 4.98 (s, 2H), 4.70 (br d, J = 4.4 Hz, |
| | 1H), 4.01 (br d, J = 4.3 Hz, 2H), 1.32 (d, J = 5.9 Hz, 3H) |
| 346 | 1H NMR (400 MHz, DMSO-d6) δ = 8.58 (d, J = 1.2 Hz, 2H), 7.12 (d, J = 1.8 |
| | Hz, 1H), 7.06-7.02 (m, 1H), 6.97-6.94 (m, 1H), 5.94 (d, J = 5.0 Hz, 1H), |
| | 5.54 (s, 2H), 5.50-5.20 (m, 2H), 4.76 (br t, J = 5.8 Hz, 1H), 4.68 (t, J = 4.6 |
| | Hz, 1H), 4.02-3.98 (m, 2H), 3.74 (s, 3H), 1.88-1.82 (m, 2H), 1.71-1.66 |
| | (m, 4H), 1.58-1.53 (m, 2H), 1.31 (d, J = 6.0 Hz, 3H) |
| 348 | 1H NMR (400 MHz, DMSO-d6) δ = 8.80 (s, 1H), 8.70 (s, 1H), 6.21 (d, J = |
| | 0.6 Hz, 1H), 5.95 (d, J = 4.9 Hz, 1H), 5.67-5.39 (m, 1H), 5.34-5.04 (m, |
| | 1H), 4.72-4.68 (m, 1H), 4.65 (s, 2H), 4.04-3.97 (m, 2H), 2.34 (s, 3H), 1.32 |
| | (d, J = 6.2 Hz, 3H) |
| 352 | 1H NMR (400 MHz, DMSO-d6) δ = 8.50 (br s, 1H), 8.44 (d, J = 2.6 Hz, 1H), |
| | 8.37 (s, 1H), 8.22 (s, 1H), 7.86 (s, 1H), 7.71 (d, J = 1.6 Hz, 1H), 7.66 (br d, J = |
| | 7.8 Hz, 1H), 7.41 (t, J = 7.8 Hz, 1H), 7.27 (d, J = 7.4 Hz, 1H), 6.52 (t, J = |
| | 2.2 Hz, 1H), 5.85 (d, J = 4.8 Hz, 1H), 5.53-5.43 (m, 1H), 5.25-5.13 (m, |
| | 1H), 4.83-4.63 (m, 3H), 4.01-3.92 (m, 2H), 1.30 (d, J = 6.0 Hz, 3H) |
| 355 | 1H NMR (400 MHz, DMSO-d6) δ = 8.20-8.19 (m, 1H), 7.47-7.44 (m, 2H), |
| | 7.40-7.36 (m, 2H), 7.35-7.31 (m, 1H), 6.88 (s, 2H), 5.71-5.68 (m, 1H), |
| | 5.53 (s, 2H), 5.28 (d, J = 5.4 Hz, 1H), 5.04 (d, J = 5.8 Hz, 2H), 4.14-4.11 |
| | (m, 1H), 3.89-3.84 (m, 1H), 1.27-1.25 (m, 3H) |
| 358 | 1H NMR (400 MHz, DMSO-d6) δ = 8.59 (s, 1H), 8.50 (s, 1H), 8.16 (s, 1H), |
| | 7.76-7.68 (m, 2H), 7.55-7.46 (m, 3H), 6.51 (s, 1H), 5.93 (d, J = 4.8 Hz, |
| | 1H), 5.65 (s, 2H), 5.55-5.36 (m, 1H), 5.34-5.11 (m, 1H), 4.69 (br t, J = 4.2 |
| | Hz, 1H), 4.04-3.95 (m, 2H), 1.31 (br d, J = 5.4 Hz, 3H) |
| 359 | 1H NMR (400 MHz, DMSO-d6) δ = 8.82 (s, 1H), 8.71 (s, 1H), 7.91 (d, J = |
| | 1.4 Hz, 1H), 7.12 (d, J = 3.6 Hz, 1H), 6.76 (s, 1H), 6.69 (dd, J = 1.8, 3.4 Hz, |
| | 1H), 5.96 (d, J =4.8 Hz, 1H), 5.50 (d, J = 5.6 Hz, 1H), 5.24-5.19 (m, 1H), |
| | 4.75 (s, 2H), 4.71 (q, J = 5.0 Hz, 1H), 4.06-3.97 (m, 2H), 1.36-1.29 (m, |
| | 3H) |
| 362 | 1H NMR (400 MHz, DMSO-d6) δ = 8.60 (s, 1H), 8.55 (s, 1H), 8.53 (d, J = |
| | 2.4 Hz, 1H), 8.07-8.01 (m, 1H), 5.94 (d, J = 5.0 Hz, 1H), 5.76 (d, J = 1.6 |
| | Hz, 2H), 5.49 (br s, 1H), 5.20 (br s, 1H), 4.69 (br s, 1H), 4.00 (br d, J = 4.5 |
| | Hz, 2H), 1.32 (d, J = 6.3 Hz, 3H) |
| 363 | 1H NMR (400 MHz, DMSO-d6) δ = 8.82-8.37 (m, 2H), 7.16-6.95 (m, 2H), |
| | 6.92-6.73 (m, 1H), 6.04-5.84 (m, 1H), 5.50 (br s, 3H), 5.20 (br s, 1H), 4.75- |
| | 4.60 (m, 1H), 4.24 (br s, 4H), 4.00 (br d, J = 4.4 Hz, 2H), 1.31 (br d, J = 2.4 |
| | Hz, 3H) |
| 364 | 1H NMR (400 MHz, DMSO-d6) δ = 8.66-8.55 (m, 4H), 7.85 (d, J = 8.1 Hz, |
| | 2H), 7.74-7.70 (m, 2H), 7.65 (d, J = 8.1 Hz, 2H), 5.95 (d, J = 5.0 Hz, 1H), |
| | 5.72 (s, 2H), 5.48 (br s, 1H), 5.20 (br s, 1H), 4.70 (br s, 1H), 4.00 (br d, J = |
| | 4.4 Hz, 2H), 1.32 (br d, J = 6.1 Hz, 3H) |
| 365 | 1H NMR (400 MHz, DMSO-d6) δ = 8.80 (s, 1H), 8.67 (s, 1H), 7.20 (t, J = |
| | 7.8 Hz, 1H), 7.02-6.96 (m, 2H), 6.79 (dd, J = 2.0, 8.3 Hz, 1H), 5.94 (d, J = |
| | 4.9 Hz, 1H), 5.50 (d, J = 5.5 Hz, 1H), 5.22 (d, J = 4.9 Hz, 1H), 4.69 (q, J = |
| | 4.8 Hz, 1H), 4.62 (s, 2H), 4.56 (td, J = 6.0, 12.0 Hz, 1H), 4.04-3.97 (m, 2H), |
| | 1.32 (d, J = 5.9 Hz, 3H), 1.25-1.23 (m, 3H), 1.22 (s, 3H) |
| 366 | 1H NMR (400 MHz, DMSO-d6) δ = 8.80 (s, 1H), 8.68 (s, 1H), 7.76-7.71 |
| | (m, 2H), 7.71-7.66 (m, 2H), 5.95 (br s, 1H), 5.49 (br s, 1H), 5.21 (br s, 1H), |
| | 4.78 (br s, 2H), 4.70 (br s, 1H), 4.01 (br s, 2H), 2.58 (s, 6H), 1.32 (br s, 3H) |
| 367 | 1H NMR (400 MHz, DMSO-d6) δ = 8.59 (d, J = 7.6 Hz, 2H), 8.18 (d, J = 1.9 |
| | Hz, 1H), 7.88-7.85 (m, 1H), 7.58 (d, J = 8.4 Hz, 1H), 5.95 (d, J = 5.0 Hz, |
| | 1H), 5.75 (s, 2H), 5.65-5.09 (m, 2H), 4.70 (t, J = 4.7 Hz, 1H), 4.00 (br d, J = |
| | 5.0 Hz, 2H), 2.34 (s, 3H), 1.32 (d, J = 6.1 Hz, 3H) |
| 370 | 1H NMR (400 MHz, DMSO-d6) δ = 8.44 (br s, 1H), 8.36 (s, 1H), 8.22 (s, |
| | 1H), 7.41-7.30 (m, 4H), 5.86 (d, J = 4.9 Hz, 1H), 5.44 (br d, J = 3.1 Hz, 1H), |
| | 5.16 (br s, 1H), 4.85-4.61 (m, 3H), 3.97 (br d, J = 5.6 Hz, 2H), 2.99-2.85 |
| | (m, 6H), 1.30 (d, J = 6.0 Hz, 3H) |
| 372 | 1H NMR (400 MHz, DMSO-d6) δ = 8.46 (s, 1H), 8.34 (s, 1H), 7.18-7.13 |
| | (m, 1H), 6.88 (dd, J = 1.2, 7.3 Hz, 1H), 6.59-6.54 (m, 2H), 5.82 (d, J = 5.0 |
| | Hz, 1H), 5.70-5.62 (m, 1H), 5.48 (d, J = 5.9 Hz, 1H), 5.22-5.16 (m, 1H), |
| | 5.09 (s, 2H), 4.54 (br dd, J = 1.9, 3.8 Hz, 1H), 4.00-3.92 (m, 2H), 2.75 (d, J = |
| | 4.8 Hz, 3H), 1.30 (d, J = 6.3 Hz, 3H) |
| 374 | 1H NMR (400 MHz, DMSO-d6) δ = 8.39-8.29 (m, 1H), 8.20 (s, 1H), 8.15 |
| | (br d, J = 3.8 Hz, 1H), 8.04 (br s, 1H), 7.55-7.44 (m, 2H), 7.39-7.31 (m, |
| | 2H), 7.30-7.23 (m, 1H), 5.85 (d, J = 4.9 Hz, 1H), 5.43 (d, J = 5.6 Hz, 1H), |
| | 5.15 (d, J = 5.3 Hz, 1H), 4.93-4.78 (m, 2H), 4.67 (br d, J = 4.9 Hz, 1H), 4.02- |
| | 3.92 (m, 2H), 1.30 (d, J = 6.1 Hz, 3H) |
| 375 | 1H NMR (400 MHz, DMSO-d6) δ = 8.63 (s, 1H), 8.59 (s, 1H), 8.03 (d, J = |
| | 7.8 Hz, 1H), 7.81-7.76 (m, 2H), 7.71-7.66 (m, 1H), 6.00 (s, 2H), 5.95 (d, J = |
| | 5.0 Hz, 1H), 5.51 (br d, J = 5.4 Hz, 1H), 5.23 (br s, 1H), 4.71 (br d, J = 4.0 |
| | Hz, 1H), 4.01 (br d, J = 4.6 Hz, 2H), 3.39 (s, 3H), 1.32 (br d, J = 6.0 Hz, 3H) |
| 376 | 1H NMR (400 MHz, DMSO-d6) δ = 8.84 (br dd, J = 1.5, 4.0 Hz, 1H), 8.60- |
| | 8.52 (m, 1H), 8.37 (br s, 1H), 8.32 (br d, J = 8.3 Hz, 1H), 8.22 (s, 1H), 7.96 |
| | (br d, J = 8.7 Hz, 1H), 7.87-7.74 (m, 2H), 7.52-7.44 (m, 1H), 5.86 (d, J = |
| | 5.0 Hz, 1H), 5.43 (br d, J = 5.9 Hz, 1H), 5.16 (br d, J = 5.1 Hz, 1H), 4.99- |
| | 4.86 (m, 2H), 4.73-4.64 (m, 1H), 4.01-3.92 (m, 2H), 1.30 (br d, J = 6.0 Hz, |
| | 3H) |
| 377 | 1H NMR (400 MHz, DMSO-d6) δ = 8.55 (s, 1H), 8.28 (s, 1H), 7.21 (d, J = |
| | 8.4 Hz, 2H), 6.47 (d, J = 8.5 Hz, 2H), 5.80 (d, J = 5.0 Hz, 1H), 5.56-5.17 |
| | (m, 2H), 5.08 (s, 2H), 4.56 (t, J = 5.0 Hz, 1H), 3.99-3.91 (m, 2H), 3.17 (br |
| | t, J = 6.1 Hz, 4H), 1.92 (br t, J = 6.3 Hz, 4H), 1.29 (d, J = 6.4 Hz, 3H) |
| 378 | 1H NMR (400 MHz, DMSO-d6) δ = 8.52 (br d, J = 3.7 Hz, 1H), 8.40 (s, 1H), |
| | 8.24 (s, 1H), 7.77-7.67 (m, 2H), 7.44 (t, J = 9.2 Hz, 1H), 5.86 (d, J = 4.9 Hz, |
| | 1H), 4.89-4.57 (m, 3H), 4.02-3.93 (m, 2H), 1.30 (d, J = 6.1 Hz, 3H) |
| 381 | 1H NMR (400 MHz, DMSO-d6) δ = 8.60 (d, J = 5.5 Hz, 2H), 7.67 (br d, J = |
| | 8.3 Hz, 2H), 7.48 (d, J = 8.3 Hz, 2H), 7.28 (s, 1H), 6.91 (s, 1H), 5.95 (d, J = |
| | 5.0 Hz, 1H), 5.72 (s, 2H), 5.47 (d, J = 5.7 Hz, 1H), 5.20 (d, J = 5.0 Hz, 1H), |
| | 4.70 (q, J = 5.3 Hz, 1H), 4.00 (br d, J = 3.8 Hz, 2H), 2.29 (s, 3H), 1.32 (br d, |
| | J = 5.9 Hz, 3H) |
| 382 | 1H NMR (400 MHz, DMSO-d6) δ = 8.56 (s, 1H), 8.42 (s, 1H), 6.88 (s, 2H), |
| | 6.06-6.01 (m, 1H), 5.62 (s, 2H), 4.78 (t, J = 4.6 Hz, 1H), 4.16-4.10 (m, |
| | 2H), 3.85 (s, 6H), 3.76 (s, 3H), 1.43 (d, J = 6.0 Hz, 3H) |
| 383 | 1H NMR (400 MHz, DMSO-d6) δ = 8.59 (d, J = 4.2 Hz, 2H), 7.81 (d, J = 8.1 |
| | Hz, 2H), 7.73 (d, J = 2.2 Hz, 1H), 7.53 (d, J = 8.1 Hz, 2H), 6.70 (d, J = 2.2 |
| | Hz, 1H), 5.95 (d, J = 5.0 Hz, 1H), 5.64 (s, 2H), 5.48 (br s, 1H), 5.20 (br s, |
| | 1H), 4.69 (br s, 1H), 4.00 (br d, J = 4.3 Hz, 2H), 3.88 (s, 3H), 1.32 (d, J = 6.0 |
| | Hz, 3H) |
| 384 | 1H NMR (400 MHz, DMSO-d6) δ = 8.66-8.61 (m, 1H), 8.50-8.42 (m, 1H), |
| | 8.36 (s, 1H), 8.23 (s, 1H), 8.01 (d, J = 8.4 Hz, 2H), 7.93-7.88 (m, 1H), 7.88- |
| | 7.82 (m, 1H), 7.44 (d, J = 8.4 Hz, 2H), 7.32 (ddd, J = 1.1, 4.8, 7.2 Hz, 1H), |
| | 5.86 (d, J = 4.9 Hz, 1H), 5.44 (d, J = 5.8 Hz, 1H), 5.16 (br d, J = 4.8 Hz, 1H), |
| | 4.81-4.71 (m, 1H), 4.71-4.65 (m, 1H), 4.01-3.93 (m, 2H), 1.30 (d, J = 6.1 |
| | Hz, 3H) |
| 385 | 1H NMR (400 MHz, DMSO-d6) δ = 8.37-8.17 (m, 3H), 7.22 (d, J = 8.6 Hz, |
| | 2H), 6.87 (d, J = 8.8 Hz, 2H), 5.85 (d, J = 4.9 Hz, 1H), 5.44 (br d, J = 5.8 Hz, |
| | 1H), 5.17 (br d, J = 5.1 Hz, 1H), 4.74-4.52 (m, 3H), 4.04-3.92 (m, 2H), |
| | 3.76-3.66 (m, 4H), 3.10-2.99 (m, 4H), 1.30 (d, J = 6.3 Hz, 3H) |
| 386 | 1H NMR (400 MHz, DMSO-d6) δ = 8.45 (br s, 1H), 8.36 (s, 1H), 8.20 (s, |
| | 1H), 8.10 (br d, J = 7.6 Hz, 1H), 7.75 (d, J =8.2 Hz, 2H), 7.38 (d, J =8.2 Hz, |
| | 2H), 5.85 (d, J =4.8 Hz, 1H), 5.42 (d, J = 5.8 Hz, 1H), 5.15 (d, J = 5.0 Hz, |
| | 1H), 4.80-4.62 (m, 3H), 4.06 (qd, J = 6.8, 13.8 Hz, 1H), 4.01-3.92 (m, 2H), |
| | 1.30 (d, J = 6.0 Hz, 3H), 1.14 (d, J = 6.6 Hz, 6H) |
| 388 | 1H NMR (400 MHz, DMSO-d6) δ = 8.35-8.33 (m, 1H), 8.25-8.18 (m, 2H), |
| | 7.25 (br d, J = 8.7 Hz, 2H), 6.85 (d, J = 8.7 Hz, 2H), 5.84 (d, J = 4.9 Hz, 1H), |
| | 4.71-4.65 (m, 1H), 4.62 (br s, 2H), 4.02-3.95 (m, 4H), 3.14 (s, 2H), 2.64 |
| | (t, J = 5.8 Hz, 2H), 2.23 (s, 6H), 1.30 (br d, J = 6.1 Hz, 3H) |
| 389 | 1H NMR (400 MHz, DMSO-d6) δ = 8.33 (s, 2H), 8.25-8.15 (m, 1H), 7.25 |
| | (d, J = 8.6 Hz, 2H), 6.89 (d, J = 8.6 Hz, 2H), 5.84 (d, J = 4.8 Hz, 1H), 5.42 |
| | (br s, 1H), 5.15 (br s, 1H), 4.71-4.55 (m, 3H), 4.49 (tt, J = 4.0, 8.6 Hz, 1H), |
| | 4.01-3.91 (m, 2H), 3.82 (td, J = 4.4, 11.6 Hz, 2H), 3.44 (ddd, J = 2.8, 9.4, |
| | 11.8 Hz, 2H), 1.92 (br dd, J = 3.8, 13.2 Hz, 2H), 1.60-1.47 (m, 2H), 1.30 (d, |
| | J = 6.0 Hz, 3H) |
| 390 | 1H NMR (400 MHz, DMSO-d6) δ = 8.80 (s, 1H), 8.67 (s, 1H), 7.55-7.41 |
| | (m, 4H), 5.94 (d, J = 4.9 Hz, 1H), 5.49 (d, J = 5.6 Hz, 1H), 5.20 (d, J = 5.0 |
| | Hz, 1H), 4.75-4.66 (m, 3H), 4.06-3.98 (m, 2H), 3.44 (br t, J = 6.7 Hz, 2H), |
| | 3.37-3.34 (m, 2H), 1.89-1.74 (m, 4H), 1.32 (d, J = 6.1 Hz, 3H) |
| 391 | 1H NMR (400 MHz, DMSO-d6) δ = 8.79 (s, 1H), 8.68 (s, 1H), 7.80-7.64 |
| | (m, 4H), 5.94 (d, J = 4.9 Hz, 1H), 5.51 (d, J = 5.5 Hz, 1H), 5.23 (d, J = 4.9 |
| | Hz, 1H), 4.77 (s, 2H), 4.69 (br d, J = 4.9 Hz, 1H), 4.06-3.96 (m, 2H), 3.11 |
| | (br t, J = 6.7 Hz, 4H), 1.68-1.57 (m, 4H), 1.32 (d, J = 6.0 Hz, 3H) |
| 392 | 1H NMR (400 MHz, DMSO-d6) δ = 8.74 (d, J = 14.1 Hz, 2H), 7.73-7.65 |
| | (m, 2H), 7.41-7.31 (m, 2H), 5.95 (d, J = 4.9 Hz, 1H), 5.58-5.48 (m, 1H), |
| | 5.23 (br s, 1H), 5.04 (s, 2H), 4.74-4.66 (m, 1H), 4.01 (br d, J = 4.2 Hz, 2H), |
| | 1.32 (br d, J = 6.1 Hz, 3H) |
| 393 | 1H NMR (400 MHz, DMSO-d6) δ = 9.17-9.11 (m, 1H), 8.80-8.75 (m, 1H), |
| | 8.69 (s, 1H), 7.84-7.68 (m, 1H), 7.67-7.62 (m, 1H), 5.95 (d, J = 4.9 Hz, |
| | 1H), 5.49 (d, J = 5.6 Hz, 1H), 5.21 (d, J = 5.1 Hz, 1H), 4.99-4.93 (m, 2H), |
| | 4.69 (q, J = 5.0 Hz, 1H), 4.05-3.97 (m, 2H), 1.32 (d, J = 6.0 Hz, 3H) |
| 394 | 1H NMR (400 MHz, MeOD-d4) δ = 8.73 (d, J = 4.9 Hz, 2H), 8.65 (s, 1H), |
| | 8.46 (s, 1H), 7.38 (br s, 1H), 6.02 (d, J = 4.4 Hz, 1H), 4.94 (s, 2H), 4.78 (br |
| | t, J = 4.5 Hz, 1H), 4.14-4.07 (m, 2H), 1.42 (br d, J = 5.9 Hz, 3H) |
| 395 | 1H NMR (400 MHz, DMSO-d6) δ = 8.74 (d, J = 3.7 Hz, 2H), 8.05-7.98 (m, |
| | 2H), 7.41-7.35 (m, 2H), 5.95 (d, J = 4.9 Hz, 1H), 5.50 (br d, J = 4.6 Hz, 1H), |
| | 5.21 (br d, J = 4.0 Hz, 1H), 5.06 (s, 2H), 4.70 (br d, J = 4.4 Hz, 1H), 4.04- |
| | 3.97 (m, 2H), 1.32 (br d, J = 6.1 Hz, 3H) |
| 396 | 1H NMR (400 MHz, DMSO-d6) δ = 8.79 (s, 1H), 8.67 (s, 1H), 8.25 (s, 1H), |
| | 7.80 (s, 1H), 7.39 (d, J = 9.3 Hz, 1H), 7.07 (dd, J = 1.5, 9.3 Hz, 1H), 5.95 (d, |
| | J = 4.9 Hz, 1H), 5.50 (d, J = 5.6 Hz, 1H), 5.21 (d, J = 5.0 Hz, 1H), 4.75-4.68 |
| | (m, 3H), 4.04-3.99 (m, 2H), 2.23 (s, 3H), 1.34-1.31 (m, 3H) |
| 397 | 1H NMR (400 MHz, DMSO-d6) δ = 8.73 (d, J = 3.5 Hz, 2H), 5.95 (d, J = 4.8 |
| | Hz, 1H), 5.51 (d, J = 5.6 Hz, 1H), 5.22 (d, J = 4.6 Hz, 1H), 4.94 (s, 2H), 4.70 |
| | (q, J = 4.9 Hz, 1H), 4.01 (br d, J = 4.3 Hz, 2H), 2.28 (s, 3H), 1.32 (br d, J = |
| | 5.7 Hz, 3H) |
| 398 | 1H NMR (400 MHz, MeOD-d4) δ = 8.73 (s, 1H), 8.50 (s, 1H), 6.04 (d, J = |
| | 4.5 Hz, 1H), 4.92 (s, 2H), 4.79 (t, J = 4.6 Hz, 1H), 4.17-4.11 (m, 2H), 2.49 |
| | (s, 3H), 1.45-1.41 (m, 3H) |
| 423 | 1H NMR (400 MHz, DMSO-d6) δ = 8.21 (br d, J = 7.4 Hz, 1H), 7.51-7.49 |
| | (m, 2H), 7.46-7.44 (m, 2H), 5.71 (d, J = 3.6 Hz, 1H), 5.52 (s, 2H), 5.18 (br |
| | s, 1H), 4.99-4.96 (m, 1H), 4.91-4.88 (m, 1H), 4.80-4.77 (m, 1H), 4.19- |
| | 4.14 (m, 1H), 4.10-4.07 (m, 1H), 3.81-3.77 (m, 2H), 3.49-3.44 (m, 2H), |
| | 3.42-3.39 (m, 1H), 3.19-3.07 (m, 2H), 2.03-1.98 (m, 1H), 1.22 (d, J = 6.4 |
| | Hz, 3H) |
| 424 | 1H NMR (400 MHz, DMSO-d6) δ = 8.25 (s, 1H), 7.54 (d, J = 8.6 Hz, 2H), |
| | 7.16 (d, J = 8.6 Hz, 2H), 7.01-6.64 (m, 1H), 5.76 (d, J = 4.2 Hz, 1H), 5.56 |
| | (s, 2H), 5.14 (ddd, J = 2.0, 4.2, 5.8 Hz, 1H), 4.34-4.29 (m, 1H), 4.26 (t, J = |
| | 5.8 Hz, 1H), 3.99-3.95 (m, 1H), 3.94-3.90 (m, 1H), 3.67-3.62 (m, 1H), |
| | 3.61-3.52 (m, 3H), 3.29-3.24 (m, 2H), 2.22-2.17 (m, 1H), 2.04-1.99 (m, |
| | 1H), 1.67-1.55 (m, 1H), 1.35 (d, J = 6.4 Hz, 3H), 0.94-0.85 (m, 3H) |

### II. BIOLOGY EXAMPLES

### Example II.1. Assay for ENT1 Activity

### Example II.1.a Binding Assay

### Purpose

Compounds of the present disclosure were assayed to show ability to bind to human ENT1. The assay is a competition between a compound of interest and Sahenta-DY647, an ENT1 inhibitor that emits fluorescence (Ex=630 nm, Em=670 nm). By measuring the fluorescence at the end of the assay, one can assess the binding potency of the compounds tested. The compounds of the present disclosure were tested and compared to known ENT inhibitors, dilazep and NBMPR:

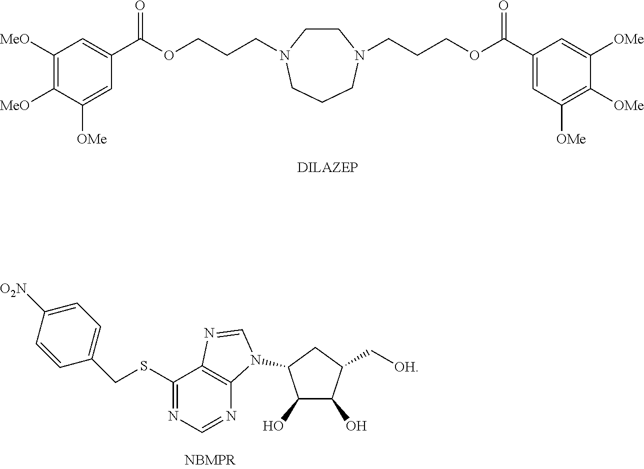 [SMILES: COc1cc(C(=O)OCCCN2CCCN(CCCOC(=O)c3cc(OC)c(OC)c(OC)c3)CC2)cc(OC)c1OC ; O=[N+]([O-])c1ccc(CSc2ncnc3c2ncn3[C@@H]2C[C@H](CO)[C@@H](O)[C@H]2O)cc1]

### Method

JAR cells expressing ENT1 were purchased from ATCC® (HTB-144TM). Cells were cultured in RPMI 1640 medium (LONZA®, #BE12-702F/U1) supplemented with 10% FBS (GIBCO®, #10270-106), 10 mM Hepes (LONZA®, #BE17-737E), 1 mM Sodium Pyruvate (LONZA®, #BE13-115E), and 2% Penicillin/Streptomycin (LONZA®, #DE17-603E) at 37° C. and 5% CO2.

The assay was conducted with the following buffer on the day of the assay: HBSS (LONZA®, #LO-527F) supplemented with 10 mM Hepes (LONZA®, #BE17-737E) and 0.1% BSA (Miltenyi®, #130-091-376).

JAR cells were resuspended in the described buffer. The compounds of interest and Sahenta-DY647 were diluted 200× with the described buffer.

A total of 50,000 cells were pre-incubated for 30 min at 4° C. with the compounds of the present disclosure before adding the corresponding amount of Sahenta-DY647 (100 nM) to the minimal concentration at Bmax and incubating for an additional 30 min at 4° C. The total volume of the reaction was 100 μL (50 μL of cells, 25 μL of the compounds of the present disclosure, and 25 μL of Sahenta-DY647) in a U-bottom 96 well plate (Greiner®, #650-180). The plates were washed 2× by centrifugation (4 min, 400 rcf at 4° C.) in the same buffer. Cells were re-suspended in 70 μL of the buffer and 50 μL was transferred to a Black 384 Optiplate (PerkinElmer®, #6007279). Fluorescence (Ex=630 nm, Em=670 nm) was acquired on a Spectramax i3x (Molecular Devices®).

### Data Analysis

Dose-response data from the compounds were analyzed with Graphpad Prism 9.1.1@software, using nonlinear regression applied to a sigmoidal dose-response model and the following equation:

Y=Bottom+(Top-Bottom)/(1+10\hat{ }((LogIC50−X)*HillSlope)), with a shared bottom and top between the test reagent and dipyridamole.

Data from multiple experiments were compiled by taking the average pIC50 value for the reagent in question±the standard deviation (s.d.) and expressing it as an average IC50 value in nM.

The inhibitory effect of the test compound was expressed as a percentage of the inhibition of SAHENTA-DY647 binding.

### Results

The potency determined in the binding assay is shown in Table 8.

The IC50 has been categorized according to the following ranges: IC50 below 0.001 μM: +++; IC50 between 0.001 and 0.02 μM: ++; IC50 between 0.02 and 0.5 μM: +; IC50 above 0.5 μM: −.

TABLE 8

| Compound | Binding |
|---|---|
| Number | Assay IC50 |
| Dilazep | ++ |
| NBMPR | +++ |
| 1 | − |
| 2 | ++ |
| 3 | + |
| 4 | + |
| 5 | ++ |
| 6 | +++ |
| 7 | ++ |
| 8 | ++ |
| 9 | +++ |
| 10 | ++ |
| 11 | +++ |
| 12 | +++ |
| 13 | + |
| 14 | +++ |
| 15 | + |
| 16 | ++ |
| 17 | ++ |
| 18 | ++ |
| 19 | + |
| 20 | ++ |
| 21 | ++ |
| 22 | ++ |
| 23 | ++ |
| 24 | ++ |
| 25 | +++ |
| 26 | ++ |
| 27 | ++ |
| 28 | +++ |
| 29 | +++ |
| 30 | +++ |
| 31 | +++ |
| 32 | + |
| 33 | + |
| 34 | + |
| 35 | +++ |
| 36 | +++ |
| 37 | ++ |
| 38 | ++ |
| 39 | ++ |
| 40 | ++ |
| 41 | + |
| 42 | ++ |
| 43 | + |
| 44 | ++ |
| 45 | ++ |
| 46 | ++ |
| 47 | ++ |
| 48 | ++ |
| 49 | ++ |
| 50 | +++ |
| 51 | ++ |
| 52 | +++ |
| 53 | +++ |
| 54 | +++ |
| 55 | +++ |
| 56 | +++ |
| 57 | +++ |
| 58 | +++ |
| 59 | +++ |
| 60 | ++ |
| 61 | + |
| 62 | ++ |
| 63 | + |
| 64 | ++ |
| 65 | ++ |
| 66 | ++ |
| 67 | +++ |
| 68 | ++ |
| 69 | + |
| 70 | ++ |
| 71 | ++ |
| 72 | ++ |
| 73 | +++ |
| 74 | ++ |
| 75 | +++ |
| 76 | ++ |
| 77 | ++ |
| 78 | ++ |
| 79 | +++ |
| 80 | +++ |
| 81 | +++ |
| 82 | ++ |
| 83 | +++ |
| 84 | +++ |
| 85 | +++ |
| 86 | +++ |
| 87 | ++ |
| 88 | ++ |
| 89 | +++ |
| 90 | ++ |
| 91 | ++ |
| 92 | ++ |
| 93 | + |
| 94 | ++ |
| 95 | ++ |
| 96 | ++ |
| 97 | ++ |
| 98 | ++ |
| 99 | ++ |
| 100 | ++ |
| 101 | + |
| 102 | ++ |
| 103 | +++ |
| 104 | ++ |
| 105 | ++ |
| 106 | ++ |
| 107 | + |
| 108 | ++ |
| 109 | ++ |
| 110 | + |
| 111 | + |
| 112 | ++ |
| 113 | ++ |
| 114 | ++ |
| 115 | ++ |
| 116 | ++ |
| 117 | ++ |
| 118 | ++ |
| 119 | + |
| 120 | +++ |
| 121 | ++ |
| 122 | ++ |
| 123 | ++ |
| 124 | +++ |
| 125 | ++ |
| 126 | ++ |
| 127 | + |
| 128 | ++ |
| 129 | ++ |
| 130 | ++ |
| 131 | ++ |
| 132 | ++ |
| 133 | ++ |
| 134 | +++ |
| 135 | + |
| 136 | +++ |
| 137 | +++ |
| 138 | +++ |
| 139 | +++ |
| 140 | +++ |
| 141 | +++ |
| 142 | +++ |
| 143 | +++ |
| 144 | ++ |
| 145 | +++ |
| 146 | ++ |
| 147 | ++ |
| 148 | ++ |
| 149 | ++ |
| 150 | ++ |
| 151 | ++ |
| 152 | ++ |
| 153 | ++ |
| 154 | ++ |
| 155 | ++ |
| 156 | ++ |
| 157 | ++ |
| 158 | ++ |
| 159 | ++ |
| 160 | ++ |
| 161 | ++ |
| 162 | ++ |
| 163 | + |
| 164 | + |
| 165 | + |
| 166 | + |
| 167 | + |
| 168 | + |
| 169 | + |
| 170 | + |
| 171 | + |
| 172 | + |
| 173 | + |
| 174 | + |
| 175 | + |
| 176 | + |
| 177 | + |
| 178 | + |
| 179 | + |
| 180 | + |
| 181 | + |
| 182 | + |
| 183 | + |
| 184 | + |
| 185 | + |
| 186 | + |
| 187 | + |
| 188 | + |
| 189 | + |
| 190 | + |
| 191 | + |
| 192 | + |
| 193 | + |
| 194 | + |
| 195 | + |
| 196 | + |
| 197 | + |
| 198 | + |
| 199 | + |
| 200 | + |
| 201 | + |
| 202 | + |
| 203 | + |
| 204 | + |
| 205 | + |
| 206 | + |
| 207 | + |
| 208 | + |
| 209 | + |
| 210 | + |
| 211 | + |
| 212 | + |
| 213 | + |
| 214 | + |
| 215 | + |
| 216 | + |
| 217 | + |
| 218 | + |
| 219 | − |
| 220 | − |
| 221 | − |
| 222 | − |
| 223 | − |
| 224 | − |
| 225 | − |
| 226 | − |
| 227 | − |
| 228 | − |
| 229 | − |
| 230 | − |
| 231 | − |
| 232 | − |
| 233 | − |
| 234 | − |
| 235 | − |
| 236 | − |
| 237 | − |
| 238 | − |
| 239 | − |
| 240 | − |
| 241 | − |
| 242 | − |
| 243 | − |
| 244 | − |
| 245 | − |
| 246 | − |
| 247 | − |
| 248 | − |
| 249 | − |
| 250 | − |
| 251 | − |
| 252 | − |
| 253 | − |
| 254 | − |
| 255 | − |
| 256 | − |
| 257 | − |
| 258 | − |
| 259 | − |
| 260 | − |
| 261 | − |
| 262 | − |
| 263 | − |
| 264 | − |
| 265 | − |
| 266 | − |
| 267 | − |
| 268 | − |
| 269 | − |
| 270 | − |
| 271 | − |
| 272 | − |
| 273 | − |
| 274 | − |
| 275 | − |
| 276 | − |
| 277 | − |
| 278 | − |
| 279 | − |
| 280 | − |
| 281 | − |
| 282 | − |
| 283 | − |
| 284 | − |
| 285 | − |
| 286 | − |
| 287 | − |
| 288 | − |
| 289 | − |
| 290 | − |
| 291 | − |
| 292 | − |
| 293 | − |
| 294 | − |
| 295 | − |
| 296 | − |
| 297 | − |
| 298 | − |
| 299 | − |
| 300 | − |
| 301 | − |
| 302 | − |
| 303 | − |
| 304 | − |
| 305 | − |
| 306 | − |
| 307 | − |
| 308 | − |
| 309 | − |
| 310 | − |
| 311 | − |
| 312 | − |
| 313 | − |
| 314 | − |
| 315 | − |
| 316 | − |
| 317 | − |
| 318 | − |
| 319 | − |
| 320 | − |
| 321 | − |
| 322 | − |
| 323 | − |
| 324 | − |
| 325 | − |
| 326 | − |
| 327 | − |
| 328 | − |
| 329 | − |
| 330 | − |
| 331 | − |
| 332 | − |
| 333 | − |
| 334 | − |
| 335 | − |
| 336 | − |
| 337 | − |
| 338 | − |
| 339 | − |
| 340 | − |
| 341 | − |
| 342 | − |
| 343 | − |
| 344 | − |
| 345 | − |
| 346 | − |
| 347 | − |
| 348 | − |
| 349 | − |
| 350 | − |
| 351 | − |
| 352 | − |
| 353 | − |
| 354 | − |
| 355 | − |
| 356 | − |
| 357 | − |
| 358 | − |
| 359 | − |
| 360 | − |
| 361 | − |
| 362 | − |
| 363 | − |
| 364 | − |
| 365 | − |
| 366 | − |
| 367 | − |
| 368 | − |
| 369 | − |
| 370 | − |
| 371 | − |
| 372 | − |
| 373 | − |
| 374 | − |
| 375 | − |
| 376 | − |
| 377 | − |
| 378 | − |
| 379 | − |
| 380 | − |
| 381 | − |
| 382 | − |
| 383 | − |
| 384 | − |
| 385 | − |
| 386 | − |
| 387 | − |
| 388 | − |
| 389 | − |
| 390 | − |
| 391 | − |
| 392 | − |
| 393 | − |
| 394 | − |
| 395 | − |
| 396 | − |
| 397 | − |
| 398 | − |
| 399 | − |
| 400 | − |
| 401 | − |
| 402 | − |
| 403 | − |
| 404 | − |
| 405 | − |
| 406 | − |
| 407 | − |
| 408 | − |
| 409 | − |
| 410 | − |
| 411 | − |
| 412 | − |
| 413 | − |
| 414 | − |
| 415 | − |
| 416 | − |
| 417 | − |
| 418 | − |
| 419 | − |
| 420 | − |
| 421 | − |
| 422 | − |
| 423 | − |
| 424 | − |

### Example II.1.b Functional Assay: Uridine Transport Inhibition Assay Purpose

The goal of this study was to determine the potency and selectivity of ENT1 inhibitors by measuring ENT1 and ENT2-mediated transport using an in vitro cellular uptake assay.

### Methods

Human ENT1 and ENT2 transporters were stably expressed in Madin-Darby Canine Kidney II (MDCKII) cells via transduction. Cell lines overexpressing the relevant ENT1 or ENT2 molecules were preincubated with a dose range of the relevant compound for 30 minutes before introduction of the specific substrate at the concentration indicated in the Table assay parameters below. The transporter assay was conducted for the time specified in the Table assay parameters before the specific uptake of the substrate into the cells was quantified. Specific uptake was determined by comparing the uptake from the cells treated with the relevant compounds and the uptake by cells not overexpressing the transporter in question.

### Data Analysis

Specific uptake for each concentration of the test sample was normalized to the DMSO control value to generate a relative accumulation value (or relative transporter activity). Data were plotted against the log molar concentration of the test reagent used. IC50 values were generated with Prism version 9.4.1 and the following function: log(inhibitor) vs. response—Variable slope (four parameters).

Table assay parameters:

| | | | | Pre- | |
|---|---|---|---|---|---|
| | | Probe | Probe | incubation | Incubation |
| Transporter | Cell line | substrate | conc. (μM) | time (mins) | time (mins) |
| ENT1 | MDCKIIENT1-LV | Uridine | 1 | 30 | 1 |
| ENT2 | MDCKIIENT2-LV | Adenosine | 0.5 | 30 | 10 |

### Results

The potency determined in the uptake assays (ENT1 and ENT2) is shown in Table 9. The IC50 has been categorized according to the following ranges: IC50 below 0.0001 μM: ++++; IC50 between 0.0001 and 0.002 μM: +++; IC50 between 0.002 and 0.02 μM: ++; IC50 between 0.02 and 1 μM: +; IC50 above 1 μM: −.

TABLE 9

| Compound | ENT1 uridine | ENT2 adenosine |
|---|---|---|
| Number | uptake assay IC50 | uptake assay IC50 |
| Dilazep | ++ | − |
| NBMPR | +++ | − |
| 147 | ++++ | − |
| 166 | +++ | − |
| 175 | +++ | − |
| 173 | +++ | − |
| 6 | +++ | + |
| 151 | +++ | − |
| 28 | +++ | − |
| 73 | +++ | + |
| 75 | +++ | + |
| 82 | +++ | + |

### Example II.1.c Functional Assay. T Cell Proliferation Assay Purpose

The goal of this study was to determine the potency of ENT1 inhibitors by measuring the rescued proliferation of stimulated primary human T cells incubated in the presence of 100 uM Adenosine triphosphate (ATP), in baseline conditions (condition A) or in the presence of various proteins known to bind small molecules (condition B).

Condition A: X-VIVO15

Condition B: X-VIVO15, 2% Human Serum Albumin (HSA) and 0.1% α-1-Acid Glycoprotein (AAG)

### Methods

### PBMC and CD3+ T Cell Isolation

Venous blood from healthy volunteers, all of whom signed an informed consent approved by the Ethics Committee (FOR—UIC—BV-050-01-01 ICF_HBS_HD Version 5.0), was obtained by ImmuneHealth (Centre Hospitalier Universitaire Tivoli, La Louviere, Belgium). Mononuclear cells were collected by density gradient centrifugation, using SepMate-50 tubes (StemCell Technologies, Grenoble, France) and Lymphoprep (Stemcell Technologies) according to the manufacturer's instructions. CD3+ T cells were isolated by immunomagnetic negative selection, using the EasySep Human T Cell Isolation Kit (StemCell Technologies) as per the manufacturer's instructions. CD3+ T cells were stored in heat inactivated FBS (hiFBS; Gibco, ThermoFisher Scientific, Merelbeke, Belgium) and 10% DMSO in liquid nitrogen.

### T Cell Proliferation Assay

Purified human CD3+ T cells were thawed and washed once with RPMI1640 medium, UltraGlutamine (Lonza, Verviers, Belgium) containing 10% hiFBS. Cells were resuspended in PBS containing 10% hiFBS at 1×10⁷ cells/ml. One volume of cells was combined with a matching volume of 2 μM CFSE solution in PBS, mixed well by pipetting, and incubated at room temperature for 5 minutes under rotation and protected from light. Excess CFSE was quenched by addition of PBS containing 10% hiFBS, and the cells were centrifuged (300×rcf, 7 minutes) and resuspended in X-VIVO15. Cells were again centrifuged and resuspended in X-VIVO15 for counting.

Cells were resuspended at 1.6×10⁶ cells/mL in X-VIVO15 medium (Condition A) or X-VIVO15 medium containing 4% (v/v) HSA (Sigma-Aldrich, Diegem, Belgium) and 0.2% (v/v) ai-Acid Glycoprotein (Sigma-Aldrich, Diegem, Belgium) (Condition B).

12.5 mL of cell suspension (2×10⁴ cells) was added to wells of sterile flat-bottom 384-well plates. Cells were activated by adding 12.5 mL of anti-CD3 anti-CD28 coated microbeads (Dynabeads human T-activator CD3/CD28; Life Technologies, Paisley, UK) suspended in X-VIVO15 medium or X-VIVO15 medium containing 4% (v/v) HSA and 0.2% (v/v) ai-Acid Glycoprotein at a ratio of one microbead per two cells. Serial dilutions of the ENT1 inhibitors were conducted in X-VIVO15 medium and 12.5 mL of the diluent was added to the wells. Cells were mixed by pipetting up and down and pre-incubated for 30 minutes in a 37° C. humidified tissue culture incubator with 5% CO2. After pre-incubation, cells were cultured in the presence or absence of ATP (Sigma-Aldrich) at a final concentration of 100 mM. The final well volume was 50 μl in all cases. Experiments were performed in biological duplicates. Cells were mixed by pipetting up and down and incubated for 4 days in a 37° C. humidified tissue culture incubator with 5% CO₂.

### Flow Cytometry Analysis

Samples were run on a BD LSR Fortessa FACS machine (BD FACSDiva Software v9.0.1). Data were analyzed using FlowJo (BD, v10.7.1). T cell proliferation was determined by measuring the frequency of ‘CFSE^{lo}’ (i.e. CFSE-diluted).

### Data Analysis

T cell proliferation data were analyzed using FlowJo software (Version 10.6.2). Dead cells and cellular debris were excluded on the basis of forward and side scatter profile and singlet cells were selected for further analysis.

T cell proliferation was determined based on the percent CFSE^{lo} total T cells for each condition. The data were plotted on the y-axis against log molar (M) concentration of ENT1 inhibitors on the x-axis in GraphPad Prism (version 9.0.0). IC50 and IC90 values were generated with the ‘log(agonist) vs. response—Find ECanything(F))’ function with F constrained to 50 or 90, respectively. pICF values were generated by taking the −logECF values.

### Reagents Used for Cell Culture and Flow Cytometry Analysis

| | | Catalogue |
|---|---|---|
| Reagent | Supplier | number |
| PBS | Lonza | BE17-516F |
| Lymphoprep ™ | Stemcell Technologies | 07861 |
| SepMate ™-50 tubes | Stemcell Technologies | 85450 |
| Fetal bovine serum (FBS) | Life Technologies | 10270-106 |
| | (GIBCO) | |
| Dimethyl sulfoxide (DMSO) | Sigma-Aldrich | D8418 |
| RPMI 1640 Medium with L- | Westburg (LONZA) | BE12-702F |
| Glutamine | | |
| EasySep ™ Human T Cell | Stemcell Technologies | 17951 |
| Isolation Kit | | |
| CFDA, SE (CFSE) | Life Technologies/ | C1157 |
| | Thermo Fisher | |
| X-VIVO 15 | Westburg (LONZA) | BE02-060F or Q |
| α₁-Acid Glycoprotein (AAG) | Sigma-Aldrich | G9885 |
| Human serum Albumin | Sigma-Aldrich | A1653 |
| (HSA) | | |
| Dynabeads human T- | Life Technologies/ | 11132D |
| activator CD3/CD28 | Thermo Fisher | |
| ATP | Sigma-Aldrich | A6419 |
| Dilazep dihydrochloride | iTeos Therapeutics | N/A |
| EDTA 0.5M | Lonza | 51234 |
| MACS BSA stock solution | Miltenyi | 130-091-376 |
| Fix Viability Dye eF660 | Invitrogen | 65-0864-14 |

### Results

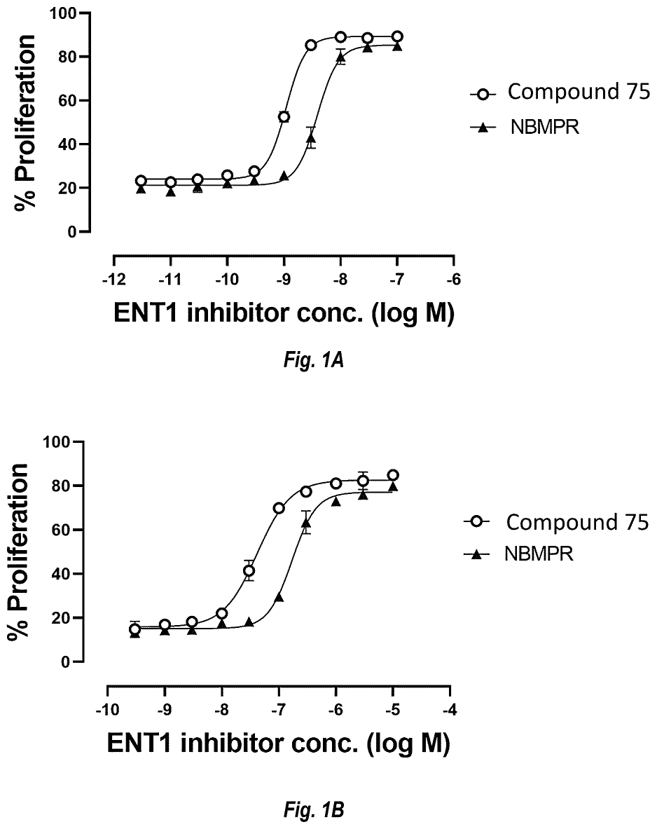

The potency determined in the T Cell proliferation assay (Condition A and B) is shown in Table 10. The IC50 has been categorized according to the following ranges: IC50 below 0.001 M: +++; IC50 between 0.001 and 0.02 M: ++; IC50 between 0.02 and 0.5 M: +; IC50 above 0.5 M: −; not determined: ND. FIG. 1 shows exemplary proliferation versus ENT1 inhibitor concentration plots determined via the T Cell proliferation assay for Compound 75 and NBMPR in both Condition A (FIG. 1A) and Condition B (FIG. 1B).

TABLE 10

| Compound | T Cell proliferation | T Cell proliferation |
|---|---|---|
| Number | (Condition A) IC50 | (Condition B) IC50 |
| Dilazep | ++ | + |
| NBMPR | ++ | + |
| 6 | +++ | ++ |
| 9 | +++ | ++ |
| 10 | +++ | ++ |
| 11 | +++ | ++ |
| 18 | +++ | ++ |
| 25 | +++ | ++ |
| 28 | +++ | ++ |
| 29 | +++ | ++ |
| 30 | +++ | ++ |
| 31 | +++ | ++ |
| 53 | +++ | ++ |
| 55 | +++ | ++ |
| 79 | +++ | ++ |
| 80 | ND | ++ |
| 2 | +++ | + |
| 8 | +++ | + |
| 12 | +++ | + |
| 14 | +++ | + |
| 16 | +++ | + |
| 20 | +++ | + |
| 23 | +++ | + |
| 27 | +++ | + |
| 40 | +++ | + |
| 47 | +++ | + |
| 54 | +++ | + |
| 73 | +++ | + |
| 75 | +++ | + |
| 77 | +++ | + |
| 81 | +++ | + |
| 82 | +++ | + |
| 89 | +++ | + |
| 112 | +++ | + |
| 113 | +++ | + |
| 114 | +++ | + |
| 151 | +++ | + |
| 153 | +++ | + |
| 157 | +++ | + |
| 5 | ++ | + |
| 17 | ++ | + |
| 21 | ++ | + |
| 22 | ++ | + |
| 24 | ++ | + |
| 26 | ++ | + |
| 34 | ++ | + |
| 44 | ++ | + |
| 65 | ++ | + |
| 108 | ++ | + |
| 145 | ++ | + |
| 147 | ++ | + |
| 150 | ++ | + |
| 156 | ++ | + |
| 158 | ++ | + |
| 161 | ++ | + |
| 162 | ++ | + |
| 166 | ++ | + |
| 173 | ++ | + |
| 175 | ++ | + |
| 179 | ++ | + |
| 49 | − | + |
| 46 | ND | + |
| 64 | ND | + |
| 66 | ND | + |
| 68 | ND | + |
| 70 | ND | + |
| 74 | ND | + |
| 76 | ND | + |
| 78 | ND | + |
| 87 | ND | + |
| 100 | ND | + |
| 103 | ND | + |
| 116 | ND | + |
| 121 | ND | + |
| 154 | ++ | − |
| 159 | ++ | − |
| 165 | ++ | − |
| 168 | ++ | − |
| 174 | ++ | − |
| 176 | ++ | − |
| 180 | ++ | − |
| 182 | ++ | − |
| 183 | ++ | − |
| 186 | ++ | − |
| 187 | ++ | − |
| 188 | ++ | − |
| 189 | ++ | − |
| 190 | ++ | − |
| 196 | ++ | − |
| 200 | ++ | − |
| 203 | ++ | − |
| 177 | + | − |
| 185 | + | − |
| 193 | + | − |
| 199 | + | − |
| 152 | − | − |
| 115 | ND | − |

LENGTHY TABLES
The patent application contains a lengthy table section. A copy of the table is available in electronic form from the USPTO web site (https://seqdata.uspto.gov/docdetail?docId=US20260184712A1). An electronic copy of the table will also be available from the USPTO upon request and payment of the fee set forth in 37 CFR 1.19(b)(3).

1. A compound of Formula (I): 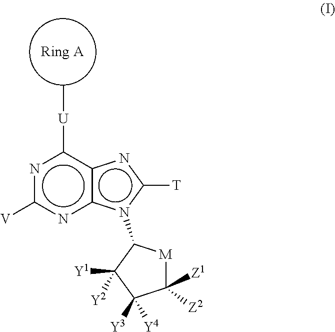
or a pharmaceutically acceptable salt, hydrate, or solvate thereof, wherein:
Ring A is chosen from optionally substituted aryl, optionally substituted heteroaryl containing at least one N, O, or S atom, optionally substituted heterocyclyl containing at least one N or O, and optionally substituted cycloalkyl;
U is a direct bond or is chosen from: (i) —O—, (ii) -alkoxy-, (iii) -(alkyl)O(alkyl)-, (iv) -alkyl-, (v) -alkenyl-, (vi) -alkyl-S— wherein the alkyl is attached to Ring A, (vii) —SO2NR¹— wherein the N is attached to Ring A, (viii) -alkyl-SO2-NR¹— wherein the N is attached to Ring A, (ix) -alkyl-NR¹— wherein the alkyl is attached to Ring A, (x) —NR¹—, (xi) —C(O)NR¹— wherein the N is attached to Ring A, (xii) —C(O)NR¹-alkyl- wherein the alkyl is attached to Ring A, and (xiii) —CO—;
T is chosen from —H, —OH, —O(alkyl)(aryl), optionally substituted heteroaryl that contains at least one N atom, —C(O)NR¹(cycloalkyl), and optionally substituted amine;
V is chosen from —H, -halo, —OH, -alkyl, and -alkoxy;
M is —O— or —C(R²)₂—;
Y¹ and Y² are each independently chosen from —H, —OH, and -halo;
Y³ is —OH or —H;
Y⁴ is chosen from —H, -halo, —OH, and -alkyl optionally substituted with 1, 2, or 3 groups independently chosen from -halo and —OH;
Z¹ is chosen from —H, -halo, —OH, and -alkyl optionally substituted with 1, 2, or 3 groups independently chosen from —OH and -halo;
Z² is chosen from —H, -alkyl, -alkenyl, —C(O)NHR¹, —C(O)NR¹(alkyl), and —C(O)O(alkyl), wherein each alkyl and alkenyl group is optionally substituted;
each R¹ is independently chosen from —H and -alkyl; and
each R² is independently chosen from —H, -alkyl, and -cycloalkyl;
on the condition that:
(a) when U is chosen from (i) —O—, (ii) -alkoxy-, and (iii) -(alkyl)O(alkyl)-, then:
at least one of Y¹, Y² or Y³ is —OH; and
when Z² is alkyl, it is not substituted with a phosphonate group or a protected alcohol group; and
with the proviso that the compound is not 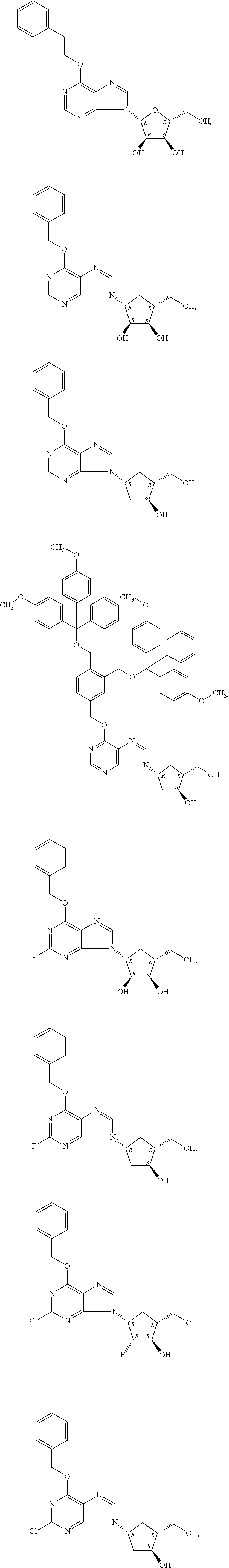 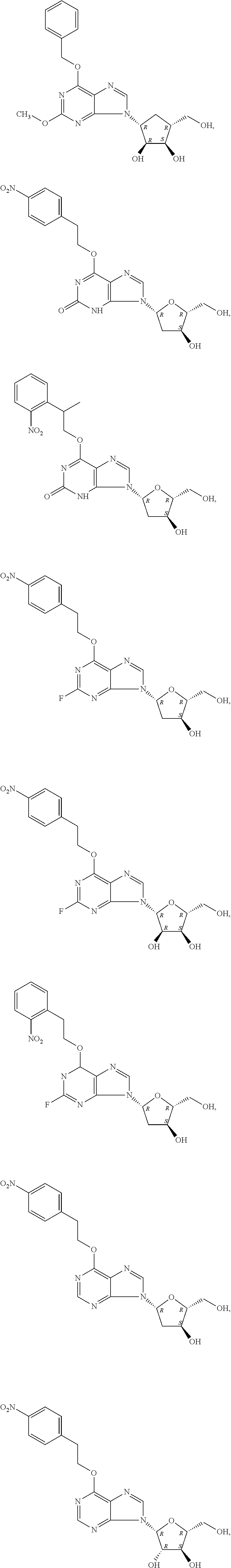 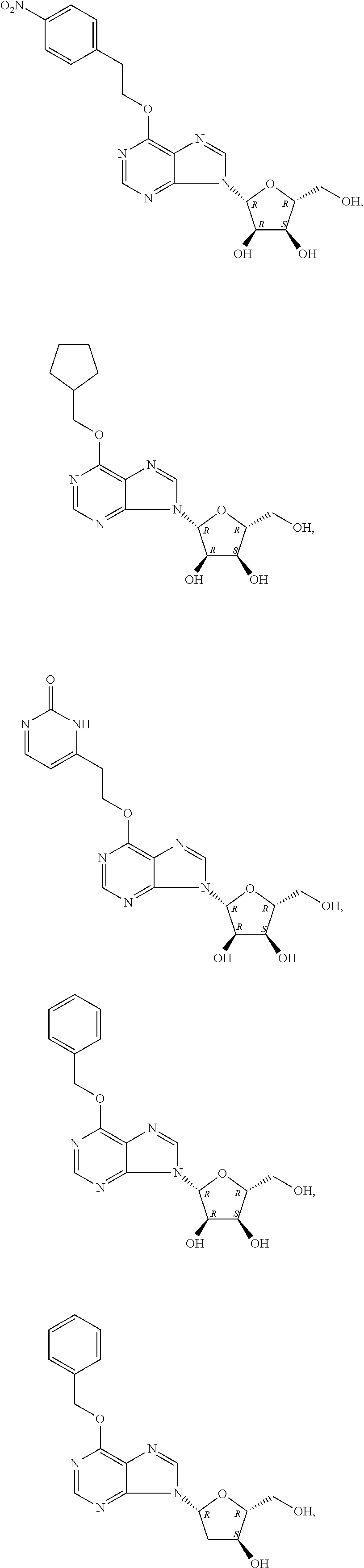 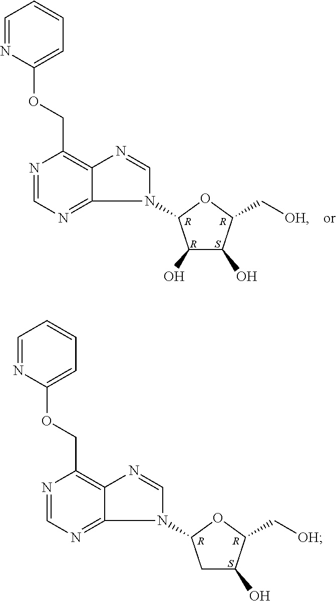
further on the condition that:
(b) when U is chosen from (iv) -alkyl- and (v) -alkenyl-, then:
Ring A is an optionally substituted aryl; and
at least one of Y¹, Y² or Y³ is —OH; and
Z² is -alkyl;
further on the condition that:
(c) when U is chosen from (ix) -alkyl-NR¹— wherein alkyl is attached to Ring A, (x) —NR¹—, (xiii) —CO—, (xi) —C(O)NR¹— wherein the N is attached to Ring A, and (xii) —C(O)NR¹-alkyl- wherein the alkyl is attached to Ring A; then:
Ring A an optionally substituted aryl; and
M is —O— or —CH₂—; and
Z² is -alkyl; and
each R¹ is independently chosen from —H and -alkyl; and
with the proviso that the compound is not 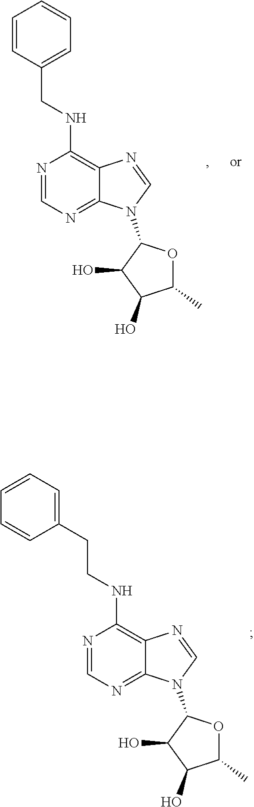 [SMILES: C[C@H]1O[C@@H](n2cnc3c(NCc4ccccc4)ncnc32)[C@H](O)[C@@H]1O ; C[C@H]1O[C@@H](n2cnc3c(NCCc4ccccc4)ncnc32)[C@H](O)[C@@H]1O]
further on the condition that:
(d) when U is chosen from: (vi) -alkyl-S— wherein alkyl is attached to Ring A, (vii) —SO₂NR¹— wherein the N is attached to Ring A, and (viii) -alkyl-SO₂—NR₁— wherein the N is attached to Ring A; then:
Ring A is C₆ aryl substituted with 1 or 2 groups independently chosen from —NO₂, —CN, —CH₂CN, alkoxy optionally substituted with 1, 2, or 3-halo atoms, and 5-membered heteroaryl optionally substituted with 1, 2, or 3 —CH₃ groups; and
T is —H; and
with the proviso that the compound is not 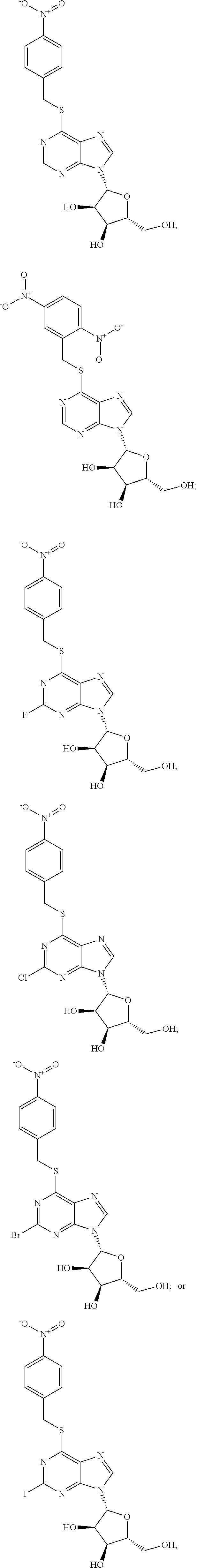
further on the condition that:
(e) when U is a direct bond, then
M is —O— or —CH₂—; and
Z² is -alkyl; and
R¹ is —H or -alkyl.

2-60. (canceled)
